(19) United States
(12) Patent Application Publication
(10) Pub. No.: US 2024/0016942 A1
(43) Pub. Date: Jan. 18, 2024

(54) STAT DEGRADERS AND USES THEREOF

(71) Applicant: Kymera Therapeutics, Inc., Watertown, MA (US)

(72) Inventors: Nan JI, Arlington, MA (US); Bin YANG, Lexington, MA (US); Xiaozhang ZHENG, Lexington, MA (US); Xiao ZHU, Winchester, MA (US)

(21) Appl. No.: 17/912,388

(22) PCT Filed: Mar. 17, 2021
(86) PCT No.: PCT/US2021/022794

§ 371 Date: Sep. 16, 2022

Related U.S. Application Data
(60) Provisional application No. 63/159,102, filed on Mar. 10, 2021.
(60) Provisional application No. 63/123,335, filed on Dec. 9, 2020.
(60) Provisional application No. 63/088,945, filed on Oct. 7, 2020.
(60) Provisional application No. 62/990,555, filed on Mar. 17, 2020.

(51) Int. Cl. A61K 47/55

(52) U.S. Cl. CPC A61K 47/55

(57) ABSTRACT

The present invention provides compounds, compositions thereof, and methods of using the same.

### CROSS-REFERENCE TO RELATED APPLICATIONS

This application claims the benefit of U.S. Provisional App. No. 62/990,555, filed Mar. 17, 2020, U.S. Provisional App. No. 63/088,945, filed Oct. 7, 2020, U.S. Provisional App. No. 63/123,335, filed Dec. 9, 2020, and U.S. Provisional App. No. 63/159,102, filed Mar. 10, 2021, the content of each of which is hereby incorporated by reference.

### TECHNICAL FIELD OF THE INVENTION

The present invention relates to compounds and methods useful for the modulation of one or more signal transducers and activators of transcription (“STAT”) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer, 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth JS Jr., Chembiochem, 2005, 6(1):40-46).

An ongoing need exists in the art for effective treatments for disease, especially hyperplasia and cancer, such as breast cancer. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as signal transducers and activators of transcription (“STAT”) hold promise as therapeutic agents. Accordingly, there remains a need to find compounds that are STAT degraders useful as therapeutic agents.

### SUMMARY OF THE INVENTION

The present application relates novel bifunctional compounds, which function to recruit STAT proteins to E3 ubiquitin ligase for degradation, and methods of preparation and uses thereof. In particular, the present disclosure provides bifunctional compounds, which find utility as modulators of targeted ubiquitination of STAT proteins, which are then degraded and/or otherwise inhibited by the bifunctional compounds as described herein. Also provided are monovalent compounds, which find utility as inducers of targeted ubiquitination of STAT proteins, which are then degraded and/or otherwise inhibited by the monovalent compounds as described herein. An advantage of the compounds provided herein is that a broad range of pharmacological activities is possible, consistent with the degradation/inhibition of STAT proteins. In addition, the description provides methods of using an effective amount of the compounds as described herein for the treatment or amelioration of a disease condition, such as cancer, e.g., breast cancer.

The present application further relates to targeted degradation of STAT proteins through the use of bifunctional molecules, including bifunctional molecules that link a cereblon-binding moiety to a ligand that binds STAT proteins.

It has now been found that compounds of this invention, and pharmaceutically acceptable compositions thereof, are effective as degraders of STAT proteins. Such compounds have the general formula I:

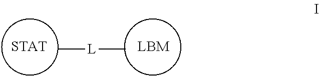

or a pharmaceutically acceptable salt thereof, wherein each variable is as defined and described herein.

It has now been found that compounds of this invention, and pharmaceutically acceptable compositions thereof, are effective for the modulation of targeted ubiquitination. Such compounds have the formula I-a to I-f:

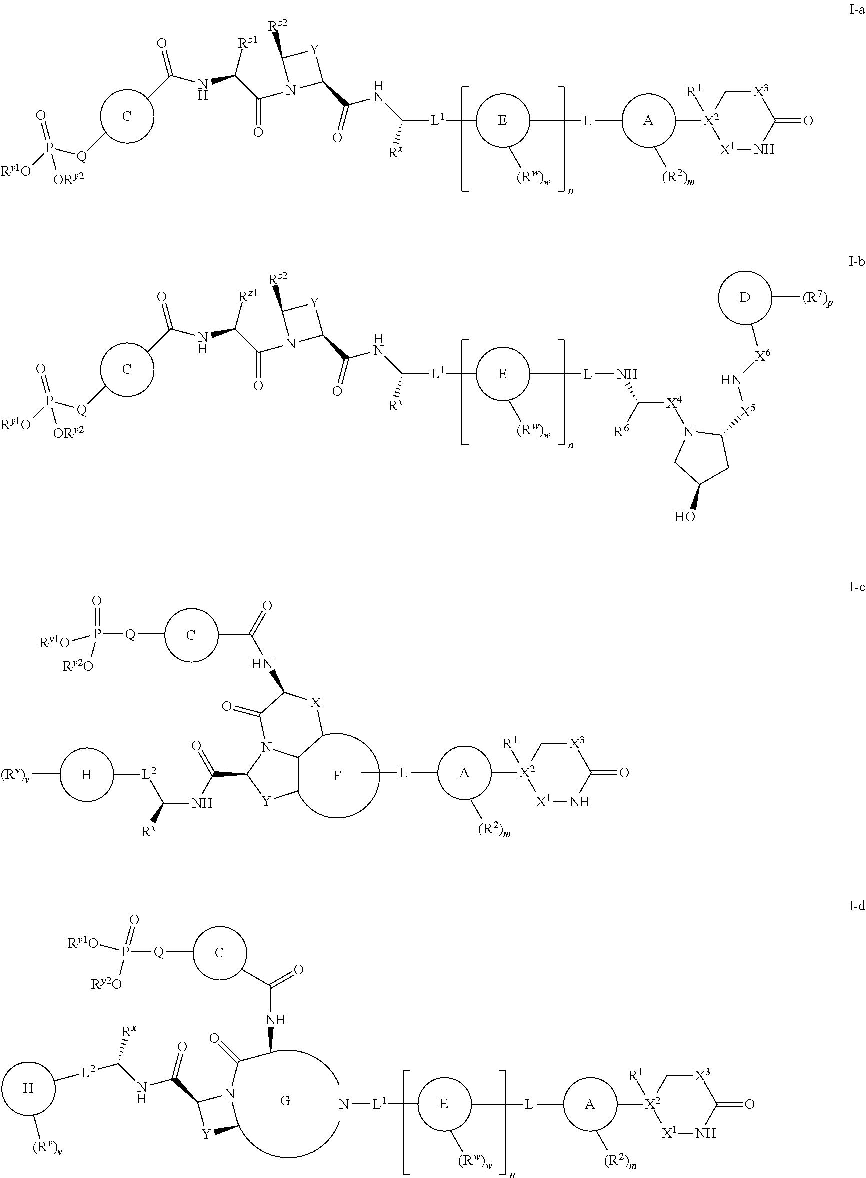

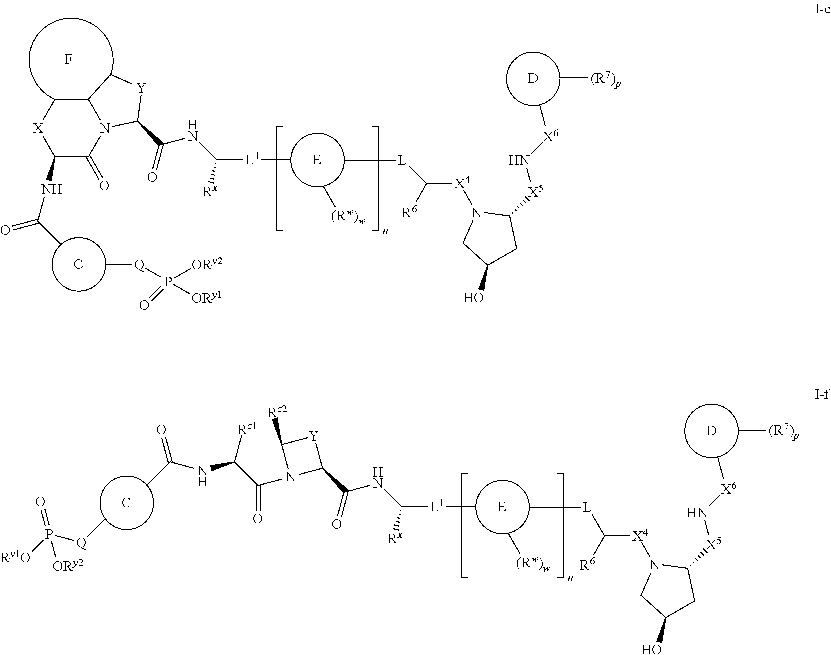

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating STAT proteins. Such diseases, disorders, or conditions include those described herein.

Compounds provided by this invention are also useful for the study of STAT proteins in biological and pathological phenomena; the study of intracellular signal transduction pathways occurring in bodily tissues; and the comparative evaluation of new STAT inhibitors or STAT degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of one or more STAT proteins. In some embodiments, a provided compound degrades and/or inhibits one or more of STAT1, STAT2, STAT3, STAT4, STAT5A, STAT5B, or STAT6.

In certain embodiments, the present invention provides a compound of formula I:

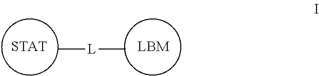

or a pharmaceutically acceptable salt thereof, wherein:
STAT is a STAT binding moiety capable of binding to STAT3;
L is a bivalent moiety that connects STAT to LBM; and
LBM is a E3 ubiquitin ligase binding moiety.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

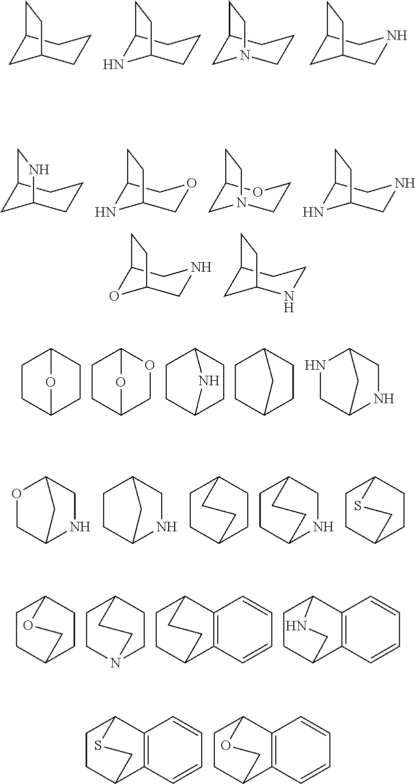

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋ₛ(or C₁-6) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

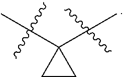 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylenyl” refers to bivalent aryl groups (e.g., phenylenyl).

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, bridged bicyclic, or spirocyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “heteroarylenyl” refers to bivalent heteroaryl groups (e.g., pyridylenyl).

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄S Rº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₄ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}3, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rº include ═0 and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{●}, —NR^{●}₂, —C(O)R^{●}, —C(O)OR^{●}, —C(O)C(O)R^{●}, —C(O)CH₂C(O)R^{●}, —S(O)₂R^{●}, —S(O)₂NR^{●}₂, —C(S)NR^{●}₂, —C(NH)NR^{●}₂, or —N(R^{●})S(O)₂R^{●}; wherein each R^{●}is independently hydrogen, C₁_aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{●}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{●} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are comtemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

As used herein, the term “inhibitor” is defined as a compound that binds to and/or inhibits an STAT protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both an STAT protein and an E3 ligase with measurable affinity resulting in the ubiquitination and subsequent degradation of the STAT protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a STAT protein activity between a sample comprising a compound of the present invention, or composition thereof, and a STAT protein, and an equivalent sample comprising a STAT protein, in the absence of said compound, or composition thereof.

### 3. Description of Exemplary Embodiments

As described above, in certain embodiments, the present invention provides a compound of formula I:

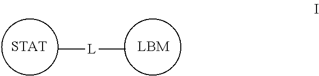

or a pharmaceutically acceptable salt thereof, wherein:
STAT is a STAT3 binding moiety;
L is a bivalent moiety that connects STAT to LBM; and
LBM is an E3 ubiquitin ligase binding moiety.

In certain embodiments, the present invention provides a compound of formula I-a:

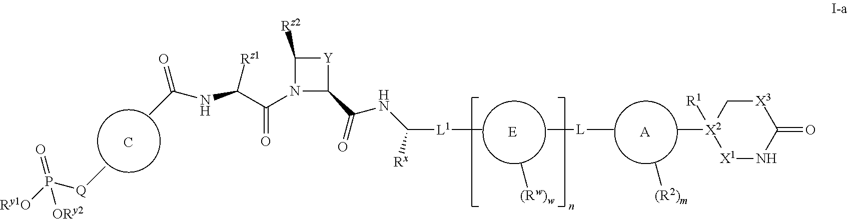

or a pharmaceutically acceptable salt thereof, wherein:
X¹ is a bivalent moiety selected from a covalent bond, —CR₂—, —C(O)—, —C(S)—, —CR(CF₃)—, —P(O)OR—, —P(O)R—P(O)NR₂—, —S(O)—, —S(O)₂—, or

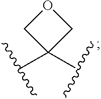

X² is a carbon atom or silicon atom;
X³ is a bivalent moiety selected from —CR₂—, —NR—, —O—, —S—, or —SiR₂—;
R¹ is hydrogen, halogen, —CN, —OR, —SR, —S(O)R, —S(O)₂R, —NR₂, —P(O)(OR)₂, —P(O)NR₂OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
each R² is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bicyclic or tricyclic ring selected from

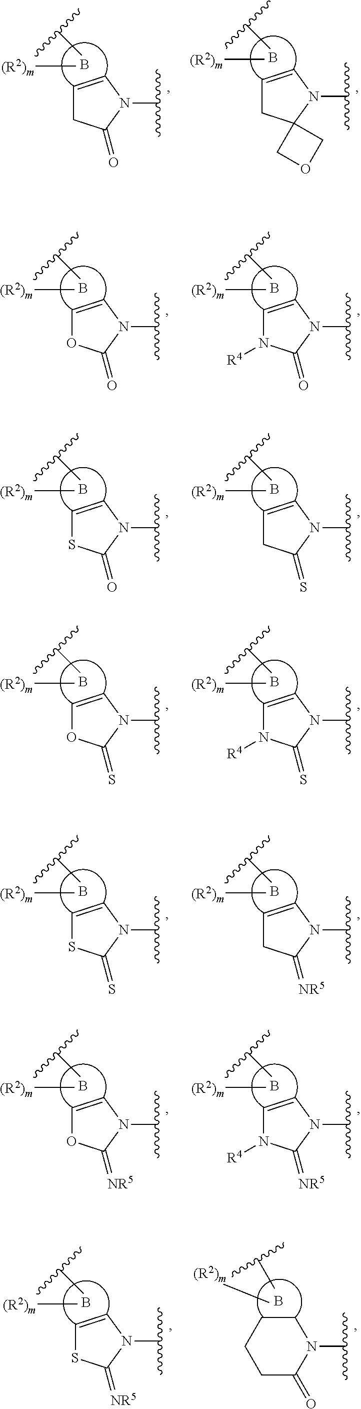

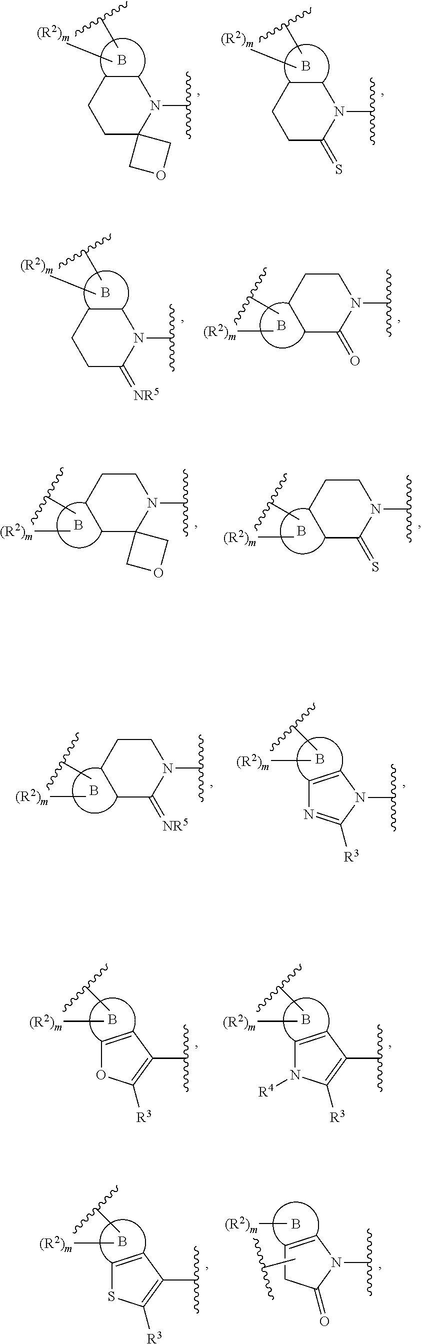

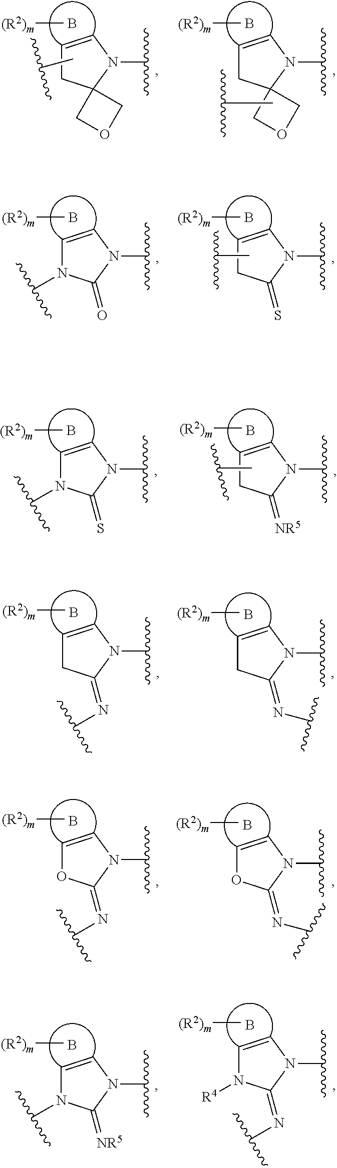

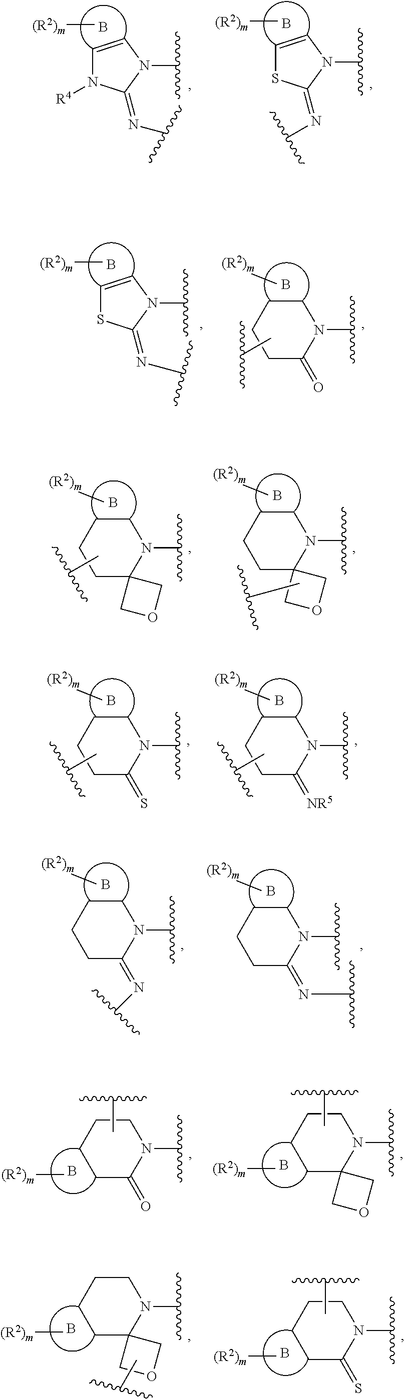

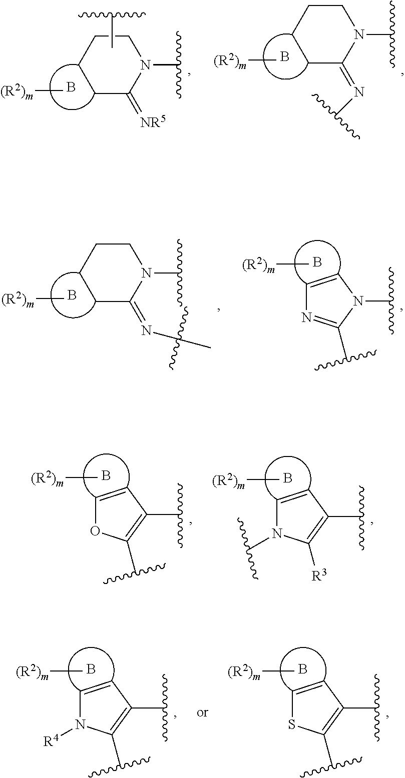

wherein:
Ring B is a fused ring selected from benzo, 5-6 membered heteroaryl containing 1-4 heteroatoms independently selected from nitrogen, oxygen, or sulfur, and a 5 to 7-membered saturated or partially unsaturated carbocyclyl or heterocyclyl with 1-3 heteroatoms independently selected from nitrogen, oxygen, or sulfur;
R³ is selected from hydrogen, halogen, —OR, —NR₂, or —SR;
each R⁴ is independently hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
R⁵ is hydrogen, C₁₋₄ aliphatic, or —CN;
m is 0, 1, 2, 3 or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
Y is an optionally substituted —(CH₂)_{y}—, wherein:
   y is 1, 2, or 3;
R^{x} is hydrogen, R^{A}, —(CR₂)₁₋₃OCONR₂, or —(CR₂)₁₋₃CONR₂;
R^{y1} and R^{y2} are each independently hydrogen, R^{A}, —CH₂CO₂R, or —CH₂OCO₂R;
R^{z1} and R^{z2} are each independently hydrogen or R^{A}, or:
   R^{z1} and R^{z2} are cyclically linked to form an optionally substituted fused 5-8 membered heterocyclic ring;
Ring C is an optionally substituted is a bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Ring E is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

In certain embodiments, the present invention provides a compound of formula I-b:

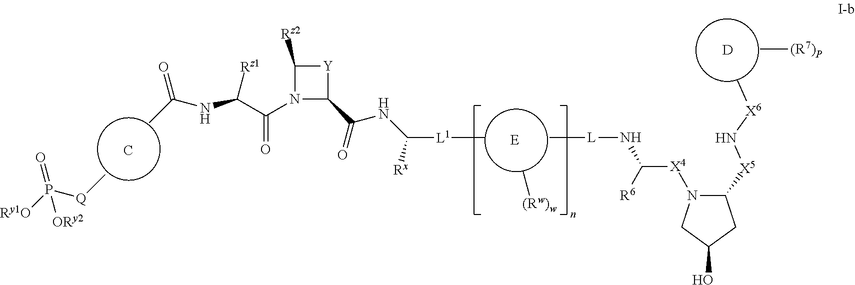

or a pharmaceutically acceptable salt thereof, wherein:
X⁴, X⁵, and X⁶ are each independently a bivalent moiety selected from a covalent bond, —CR₂—, —C(O)—, —C(S)—, —O—, —S(O)—, —S(O)₂—,

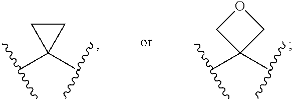

each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
R⁶ is hydrogen or R^{A};
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring D is selected from phenyl, a 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur, and a 5-6 membered heteroaryl with 1-4 heteroatoms independently selected from nitrogen, oxygen or sulfur;
R⁷ is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
p is 0, 1, 2, 3, or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
Y is an optionally substituted —(CH₂)_{y}—, wherein:
   y is 1, 2, or 3;
R^{x} is hydrogen, R^{A}, —(CR₂)₁₋₃OCONR₂, or —(CR₂)₁₋₃CONR₂;
R^{y1} and R^{y2} are each independently hydrogen, R^{A}, —CH₂CO₂R, or —CH₂OCO₂R;
R^{z1} and R^{z2} are each independently hydrogen or R^{A}, or:
   R^{z1} and R^{z2} are cyclically linked to form an optionally substituted fused 5-8 membered heterocyclic ring;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Ring E is a bivalent ring selected from phenylenyl, a 4-7 membered saturated or partially unsaturated carbocyclylenyl or heterocyclylenyl having 1-2 heteroatoms ind. having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

In certain embodiments, the present invention provides a compound of formula I-c:

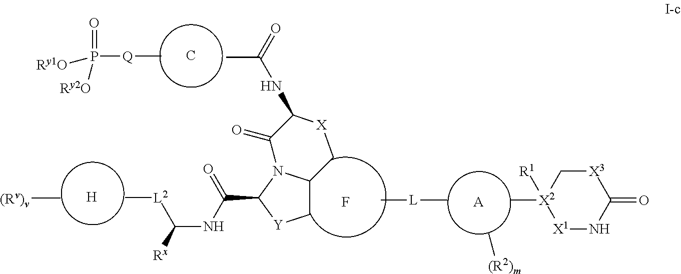

or a pharmaceutically acceptable salt thereof, wherein:
X¹ is a bivalent moiety selected from a covalent bond, —CR₂—, —C(O)—, —C(S)—, —CR(CF₃)—, —P(O)OR—, —P(O)R—P(O)NR₂—, —S(O)—, —S(O)₂—, or

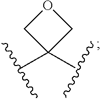

X² is a carbon atom or silicon atom;
X³ is a bivalent moiety selected from —CR₂—, —NR—, —O—, —S—, or —SiR₂—;
R¹ is hydrogen, halogen, —CN, —OR, —SR, —S(O)R, —S(O)₂R, —NR₂, —P(O)(OR)₂, —P(O)NR₂OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
each R² is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bicyclic or tricyclic ring selected from

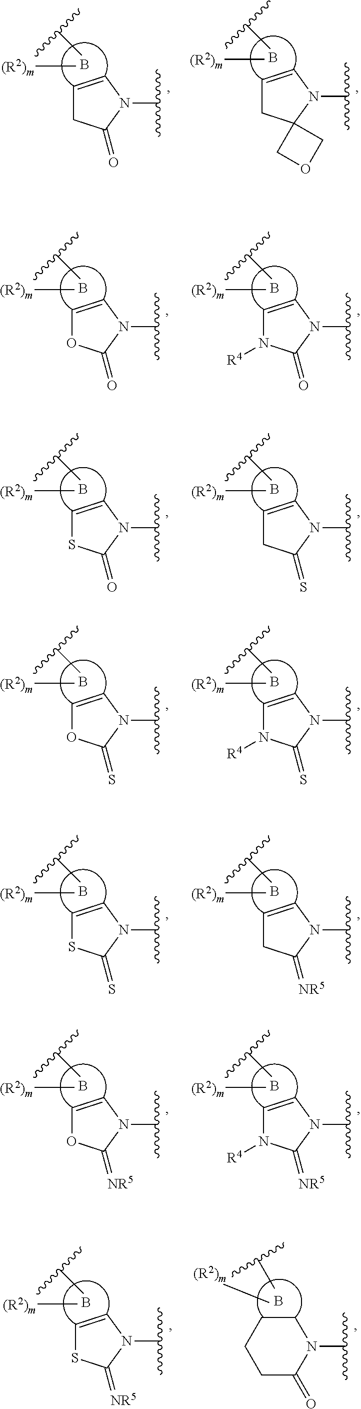

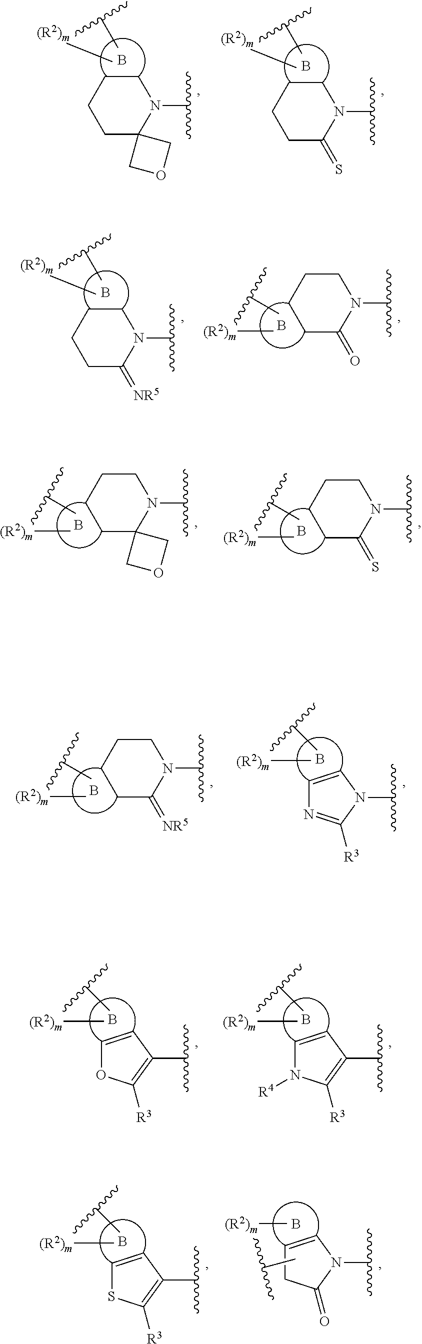

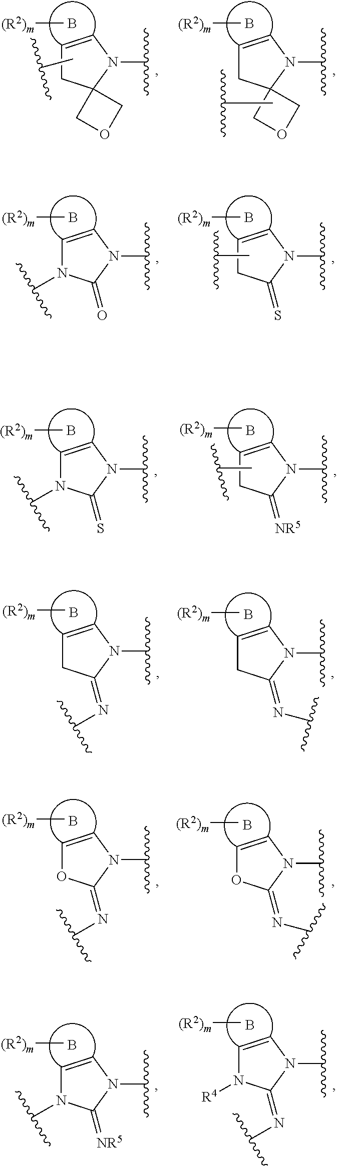

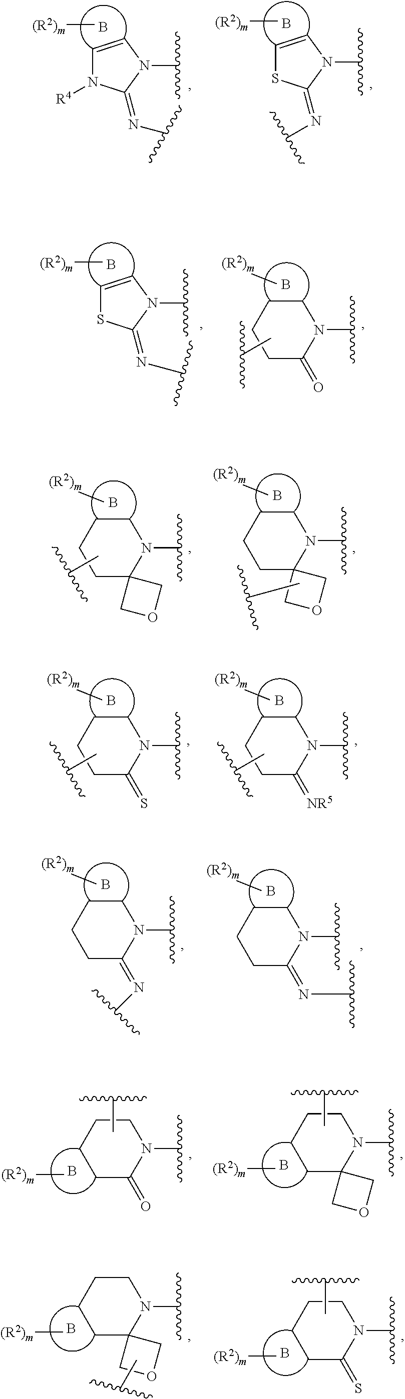

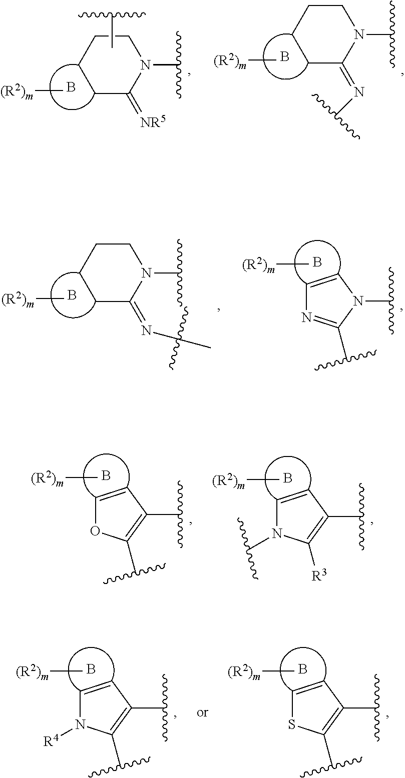

wherein:
Ring B is a fused ring selected from benzo, 5-6 membered heteroaryl containing 1-4 heteroatoms independently selected from nitrogen, oxygen, or sulfur, and a 5 to 7-membered saturated or partially unsaturated carbocyclyl or heterocyclyl with 1-3 heteroatoms independently selected from nitrogen, oxygen, or sulfur;
R³ is selected from hydrogen, halogen, —OR, —NR₂, or —SR;
each R⁴ is independently hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
R⁵ is hydrogen, C₁₋₄ aliphatic, or —CN;
m is 0, 1, 2, 3 or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L² is a covalent bond or a bivalent, saturated or partially unsaturated, straight or branched C₁₋₅ hydrocarbon chain, wherein 0-3 methylene units of L² are independently replaced by —O—, —NR—, —CRF—, —CF₂—, —C(O)—, —S—, —S(O)—, or —S(O)₂—;
R^{x} is hydrogen, R^{A}, —(CR₂)₁₋₃OCONR₂, or —(CR₂)₁₋₃CONR₂;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Q is a bivalent moiety selected from —O—, —CR₂—, —CF₂—, —CFR—, —C(O)—, —OCR₂—, and —C(S)—;
R^{y1} and R^{y2} are each independently hydrogen or R^{A};
Ring F is an optionally substituted fused ring selected from a 6-membered aryl, a 5-6 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur;
X is an optionally substituted —(CH₂)ₓ—, wherein:
   x is 0, 1, 2, or 3;
Y is an optionally substituted —(CH₂)_{y}—, wherein:
   y is 0, 1, 2, or 3;
Ring H is absent or a ring selected from phenyl, a 5-6 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur;
R^{v} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R; and
v is 0, 1, 2, 3, or 4.

In certain embodiments, the present invention provides a compound of formula I-d:

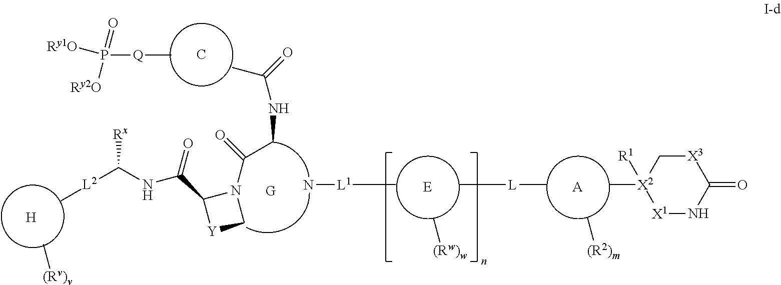

or a pharmaceutically acceptable salt thereof, wherein:
X¹ is a bivalent moiety selected from a covalent bond, —CR₂—, —C(O)—, —C(S)—, —CR(CF₃)—, —P(O)OR—, —P(O)R—P(O)NR₂—, —S(O)—, —S(O)₂—, or

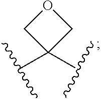

X² is a carbon atom or silicon atom;
X³ is a bivalent moiety selected from —CR₂—, —NR—, —O—, —S—, or —SiR₂—;
R¹ is hydrogen, halogen, —CN, —OR, —SR, —S(O)R, —S(O)₂R, —NR₂, —P(O)(OR)₂, —P(O)NR₂OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
each R² is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bicyclic or tricyclic ring selected from

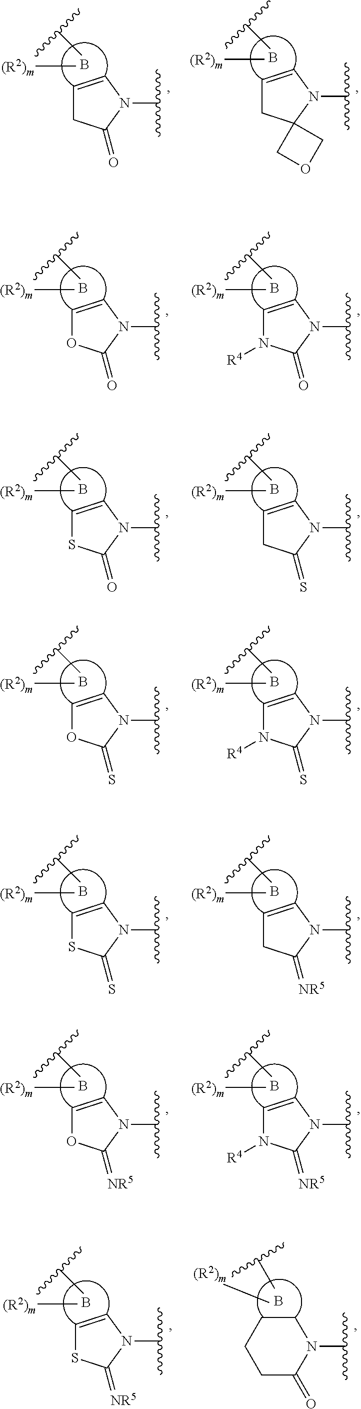

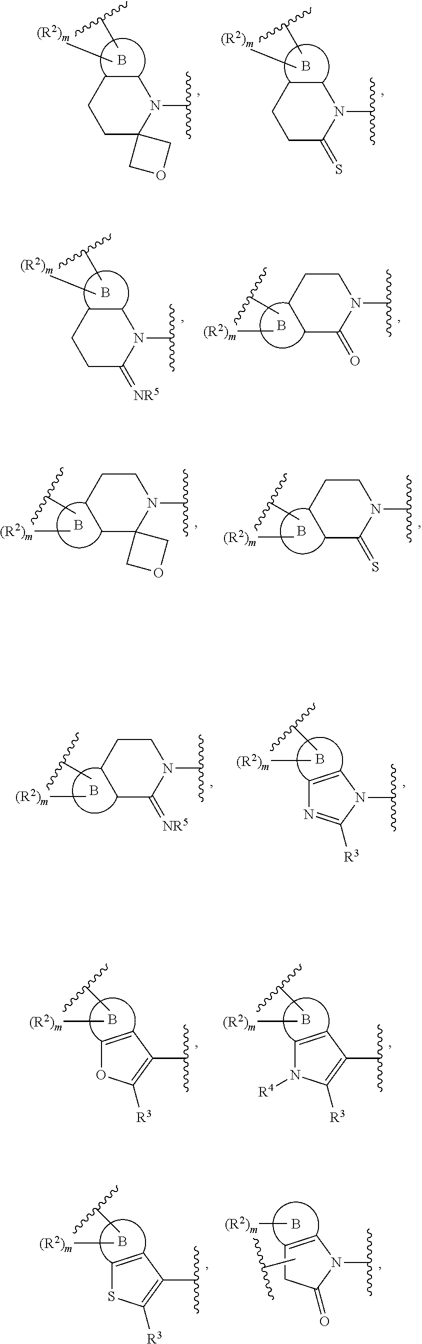

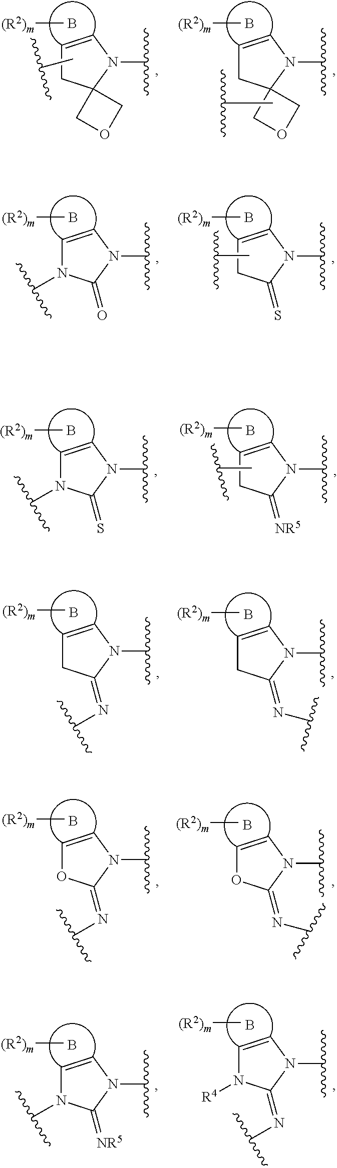

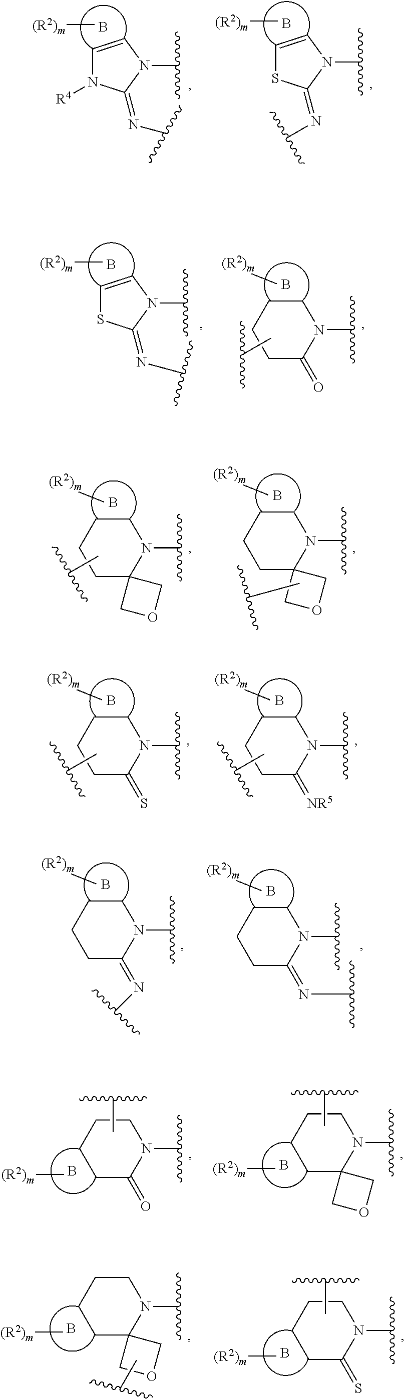

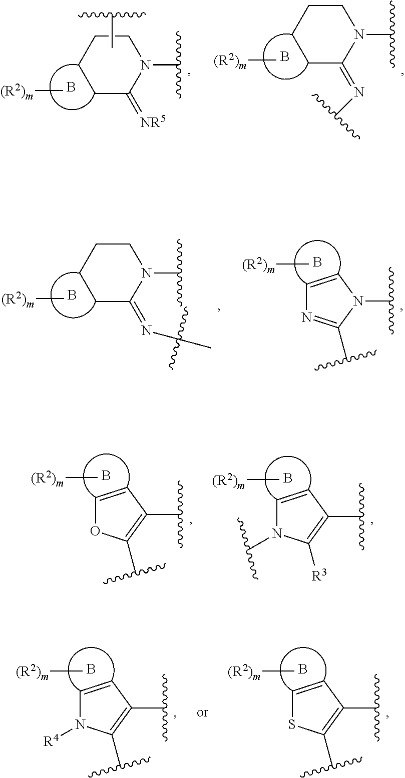

wherein:
Ring B is a fused ring selected from benzo, 5-6 membered heteroaryl containing 1-4 heteroatoms independently selected from nitrogen, oxygen, or sulfur, and a 5 to 7-membered saturated or partially unsaturated carbocyclyl or heterocyclyl with 1-3 heteroatoms independently selected from nitrogen, oxygen, or sulfur;
R³ is selected from hydrogen, halogen, —OR, —NR₂, or —SR;
each R⁴ is independently hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
R⁵ is hydrogen, C₁₋₄ aliphatic, or —CN;
m is 0, 1, 2, 3 or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋ₛ hydrocarbon chain, wherein 0-3 methylene units of L¹ are independently replaced by —O—, —NR—, —CRF—, —CF₂—, —C(O)—, —S—, —S(O)—, or —S(O)₂—;
L² is a covalent bond or a bivalent, saturated or partially unsaturated, straight or branched C₁₋ₛ hydrocarbon chain, wherein 0-3 methylene units of L² are independently replaced by —O—, —NR—, —CRF—, —CF₂—, —C(O)—,

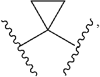

—S—, —S(O)—, or —S(O)₂—;
R^{x} is hydrogen, R^{A}, —(CR₂)₁₋₃OCONR₂, or —(CR₂)₁₋₃CONR₂;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Q is a bivalent moiety selected from —O—, —CR₂—, —CF₂—, —CFR—, —C(O)—, —OCR₂—, and —C(S)—;
R^{y1} and R^{y2} are each independently hydrogen or R^{A};
Y is an optionally substituted —(CH₂)_{y}—, wherein:
   y is 1, 2, or 3;
Ring G is an optionally substituted ring selected from a 5-9 membered saturated or partially unsaturated heterocyclyl;
Ring H is a ring selected from phenyl, a 5-9 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur;
R^{v} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
v is 0, 1, 2, 3, or 4;
Ring E is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

In certain embodiments, the present invention provides a compound of formula I-e:

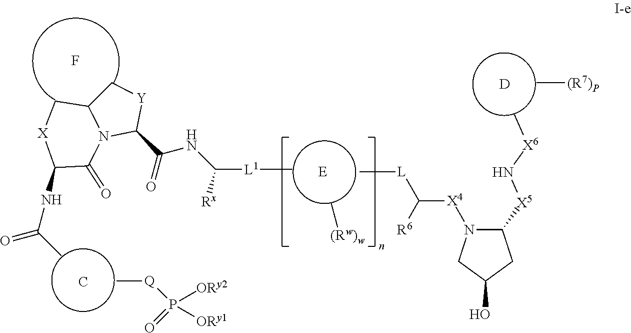

or a pharmaceutically acceptable salt thereof, wherein:
X⁴, X⁵, and X⁶ are each independently a bivalent moiety selected from a covalent bond, —CR₂—, —C(O)—, —C(S)—, —O—, —S(O)—, —S(O)₂—,

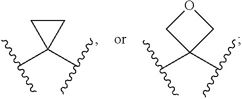 [SMILES: *C1(*)CC1 ; *C1(*)COC1]

each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
R⁶ is hydrogen or R^{A};
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring D is selected from phenyl, a 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur, and a 5-6 membered heteroaryl with 1-4 heteroatoms independently selected from nitrogen, oxygen or sulfur;
R⁷ is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
p is 0, 1, 2, 3, or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
R^{x} is hydrogen, R^{A}, —(CR₂)₁₋₃OCONR₂, or —(CR₂)₁₋₃CONR₂;
R^{y1} and R^{y2} are each independently hydrogen, R^{A}, —CH₂CO₂R, or —CH₂OCO₂R;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Ring E is a bivalent ring selected from phenylenyl, a 4-7 membered saturated or partially unsaturated carbocyclylenyl or heterocyclylenyl having 1-2 heteroatoms ind. having 1-4 heteroatoms independently selected from nitrogen, oxygen, and sulfur;
d
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

In certain embodiments, the present invention provides a compound of formula I-f:

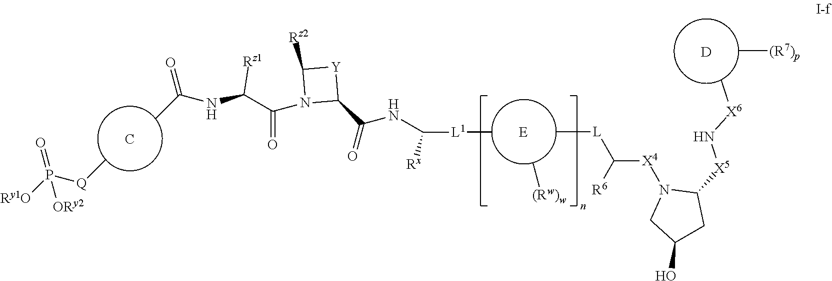

or a pharmaceutically acceptable salt thereof, wherein:
X⁴, X⁵, and X⁶ are each independently a bivalent moiety selected from a covalent bond, —CR₂—, —C(O)—, —C(S)—, —O—, —S(O)—, —S(O)₂—,

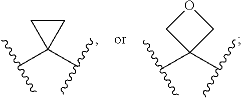 [SMILES: *C1(*)CC1 ; *C1(*)COC1]

each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
R⁶ is hydrogen or R^{A};
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring D is selected from phenyl, a 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur, and a 5-6 membered heteroaryl with 1-4 heteroatoms independently selected from nitrogen, oxygen or sulfur;
R⁷ is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
p is 0, 1, 2, 3, or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
Y is an optionally substituted —(CH₂)_{y}—, wherein:
   y is 1, 2, or 3;
R^{x} is hydrogen, R^{A}, —(CR₂)₁₋₃OCONR₂, or —(CR₂)₁₋₃CONR₂;
R^{y1} and R^{y2} are each independently hydrogen, R^{A}, —CH₂CO₂R, or —CH₂OCO₂R;
R^{z1} and R^{z2} are each independently hydrogen or R^{A}, or:
   R^{z1} and R^{z2} are cyclically linked to form an optionally substituted fused 5-8 membered heterocyclic ring;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Ring E is a bivalent ring selected from phenylenyl, a 4-7 membered saturated or partially unsaturated carbocyclylenyl or heterocyclylenyl having 1-2 heteroatoms ind. having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

As defined above and described herein, X¹ is a bivalent moiety selected from a covalent bond, —CR₂—, —C(O)—, —C(S)—, —CR(CF₃)—, —P(O)OR—, —P(O)R—, —P(O)NR₂—, —S(O)—, —S(O)₂—, or

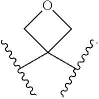 [SMILES: *C1(*)COC1]

In some embodiments, X¹ is covalent bond. In some embodiments, X¹ is —CR₂—. In some embodiments, X¹ is —C(O)—. In some embodiments, X¹ is —C(S)—. In some embodiments, X¹ is —CR(CF₃)—. In some embodiments, X¹ is —P(O)OR—. In some embodiments, X¹ is —P(O)R—. In some embodiments, X¹ is —P(O)NR₂—. In some embodiments, X¹ is —S(O)—. In some embodiments, X¹ is —S(O)₂—. In some embodiments, X¹ is

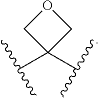 [SMILES: *C1(*)COC1]

In some embodiments, X¹ is selected from those depicted in Table 1, below.

As defined above and described herein, X² is a carbon atom or silicon atom.

In some embodiments, X² is a carbon atom. In some embodiments, X² is a silicon atom.

In some embodiments, X² is selected from those depicted in Table 1, below.

As defined above and described herein, X³ is a bivalent moiety selected from —CR₂—, —NR—, —O—, —S—, or —SiR₂—.

In some embodiments, X³ is —CR₂—. In some embodiments, X³ is —NR—. In some embodiments, X³ is —O—. In some embodiments, X³ is —S—. In some embodiments, X³ is —SiR₂—.

In some embodiments, X³ is selected from those depicted in Table 1, below.

As defined above and described herein, X⁴, X⁵, and X⁶ are each independently a bivalent moiety selected from a covalent bond, —CR₂—, —C(O)—, —C(S)—, —O—, —S(O)—, —S(O)₂—,

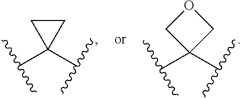 [SMILES: *C1(*)CC1 ; *C1(*)COC1]

In some embodiments, X⁴ is a covalent bond. In some embodiments, X⁴ is —CR₂—. In some embodiments, X⁴ is —C(O)—. In some embodiments, X⁴ is —C(S)—. In some embodiments, X⁴ is —O—. In some embodiments, X⁴ is —S(O)—. In some embodiments, X⁴ is —S(O)₂—. In some embodiments, X⁴ is

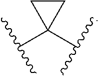 [SMILES: *C1(*)CC1]

In some embodiments, X⁴ is

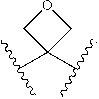 [SMILES: *C1(*)COC1]

In some embodiments, X⁵ is a covalent bond. In some embodiments, X⁵ is —CR₂—. In some embodiments, X⁵ is —C(O)—. In some embodiments, X⁵ is —C(S)—. In some embodiments, X⁵ is —O—. In some embodiments, X⁵ is —S(O)—. In some embodiments, X⁵ is —S(O)₂—.

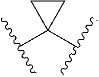 [SMILES: *C1(*)CC1]

In some embodiments, X⁵ is

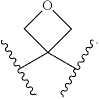 [SMILES: *C1(*)COC1]

In some embodiments, X⁵ is. In some embodiments, X⁶ is a covalent bond. In some embodiments, X⁶ is —CR₂—. In some embodiments, X⁶ is —C(O)—. In some embodiments, X⁶ is —C(S)—. In some embodiments, X⁶ is —O—. In some embodiments, X⁶ is —S(O)—. In some embodiments, X⁶ is —S(O)₂—. In some embodiments, X⁶ is

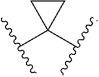 [SMILES: *C1(*)CC1]

In some embodiments, X⁶ is

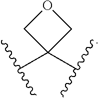 [SMILES: *C1(*)COC1]

In some embodiments, X⁶ is

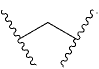 [SMILES: *C*]

In some embodiments, X⁶ is

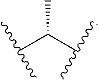

As defined above and described herein, R¹ is hydrogen, halogen, —CN, —OR, —SR, —S(O)R, —S(O)₂R, —NR₂, —P(O)(OR)₂, —P(O)NR₂OR, —P(O)(NR₂)₂, —Si(OH)₂R, —Si(OH)R₂, —SiR₃, or an optionally substituted C₁₋₄ aliphatic.

In some embodiments, R¹ is hydrogen. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)NR₂OR. In some embodiments,
R¹ is —P(O)(NR₂)₂. In some embodiments, R¹ is —Si(OH)₂R, —Si(OH)R₂. In some embodiments, R¹ is —SiR₃. In some embodiments, R¹ is an optionally substituted C₁₋₄ aliphatic.

In some embodiments, R¹ is selected from those depicted in Table 1, below.

As defined above and described herein, each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 3-7 membered saturated or partially unsaturated heterocyclic having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur.

In some embodiments, R is selected from those depicted in Table 1, below.

As defined above and described herein, each R² is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R.

In some embodiments, R² is hydrogen. In some embodiments, R² is R^{A}. In some embodiments, R² is halogen. In some embodiments, R² is —CN. In some embodiments, R² is —NO₂. In some embodiments, R² is —OR. In some embodiments, R² is —SR. In some embodiments, R² is —NR₂. In some embodiments, R² is —SiR₃. In some embodiments, R² is —S(O)₂R. In some embodiments, R² is —S(O)₂NR₂. In some embodiments, R² is —S(O)R, —C(O)R. In some embodiments, R² is —C(O)OR. In some embodiments, R² is —C(O)NR₂. In some embodiments, R² is —C(O)NROR. In some embodiments, R² is —CR₂NRC(O)R. In some embodiments, R² is —CR₂NRC(O)NR₂. In some embodiments, R² is —OC(O)R. In some embodiments, R² is —OC(O)NR₂. In some embodiments, R² is —OP(O)R₂. In some embodiments, R² is —OP(O)(OR)₂. In some embodiments, R² is —OP(O)(OR)NR₂. In some embodiments, R² is —OP(O)(NR₂)₂. In some embodiments, R² is —NRC(O)OR. In some embodiments, R² is —NRC(O)R. In some embodiments, R² is —NRC(O)NR₂. In some embodiments, R² is —NRS(O)₂R. In some embodiments, R² is —NP(O)R₂. In some embodiments, R² is —NRP(O)(OR)₂. In some embodiments, R² is —NRP(O)(OR)NR₂. In some embodiments, R² is —NRP(O)(NR₂)₂. In some embodiments, R² is —NRS(O)₂R.

In some embodiments, R² is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

As defined above and described herein, each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 4-7 membered saturated or partially unsaturated carbocyclic or heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur. In some embodiments, R^{A} is

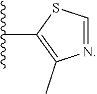 [SMILES: *c1scnc1C]

In some embodiments, R^{A} is selected from those depicted in Table 1, below.

As defined above and described herein, Ring A is a bicyclic or tricyclic ring selected from

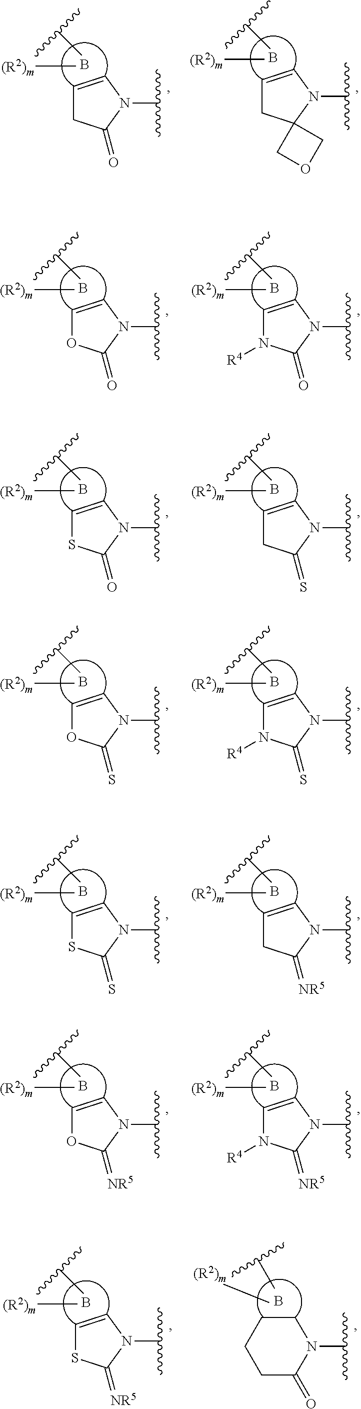

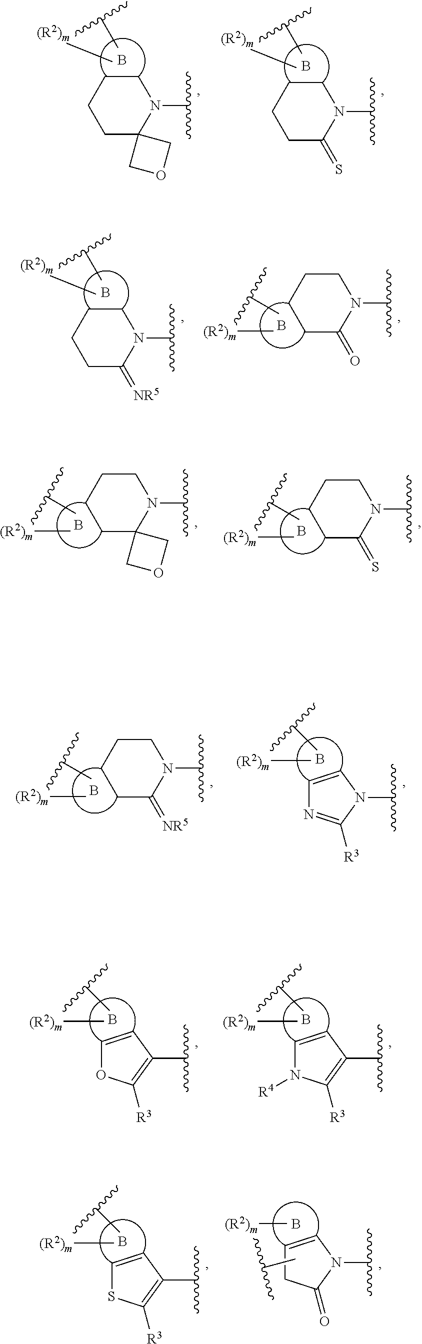

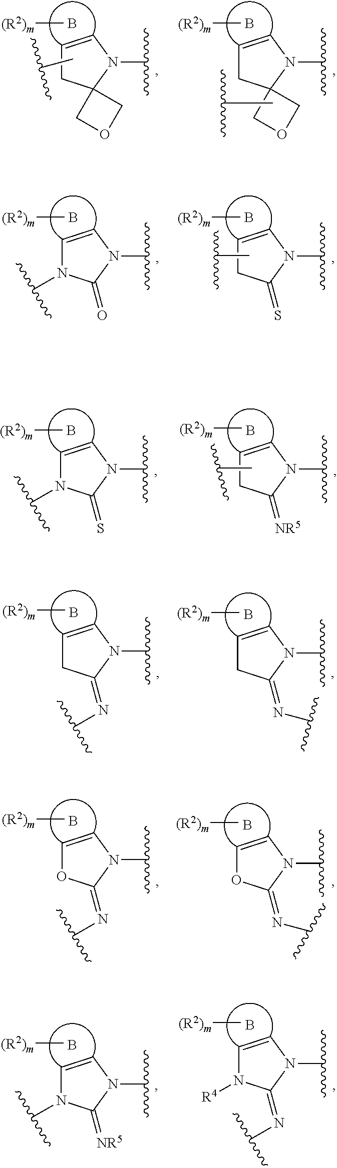

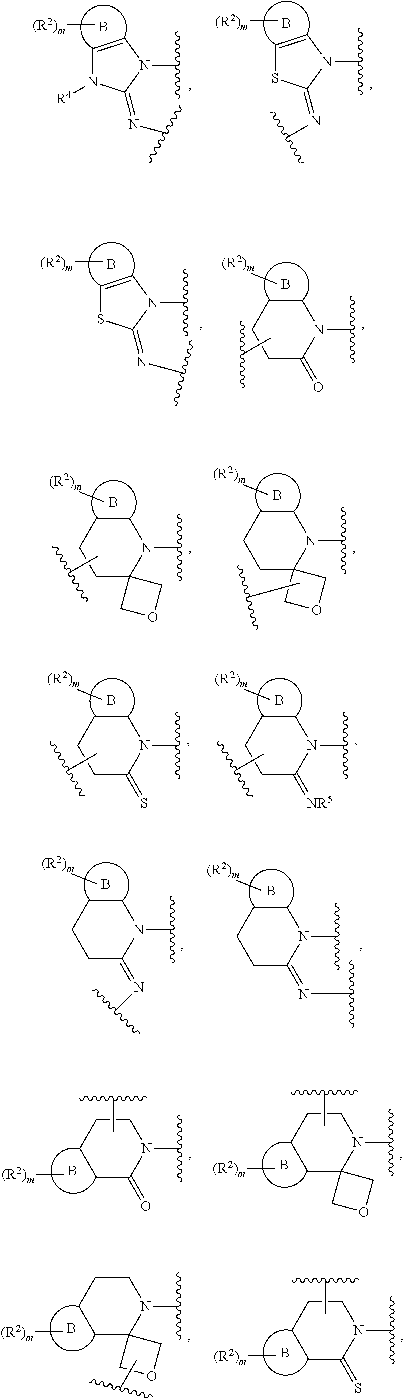

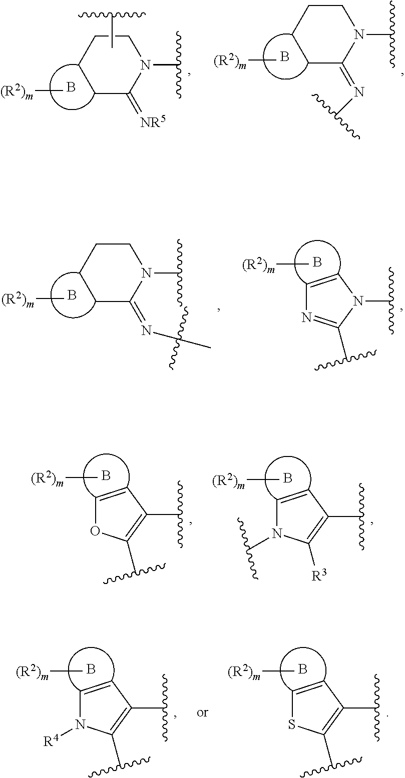

In some embodiments, Ring A is

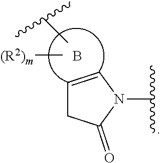

In some embodiments, Ring A is

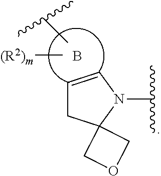

In some embodiments, Ring A is

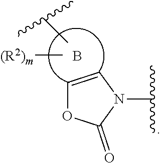

In some embodiments, Ring A is

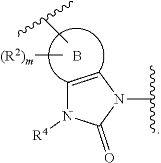

In some embodiments, Ring A is

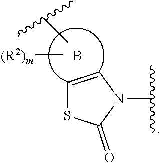

In some embodiments, Ring A is

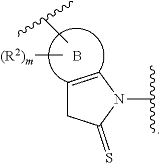

In some embodiments, Ring A is

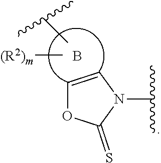

In some embodiments, Ring A is

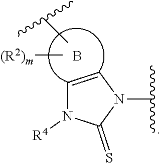

In some embodiments, Ring A is

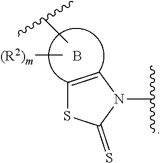

In some embodiments, Ring A is

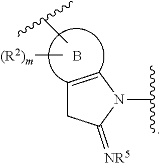

In some embodiments, Ring A is

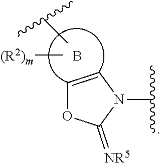

In some embodiments, Ring A is

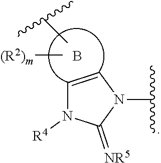

In some embodiments, Ring A is

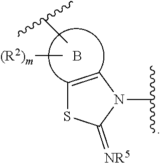

In some embodiments, Ring A is

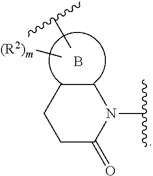

In some embodiments, Ring A is

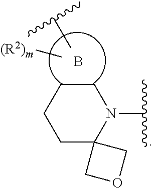

In some embodiments, Ring A is

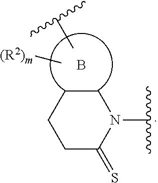

In some embodiments, Ring A is

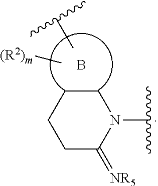

In some embodiments, Ring A is

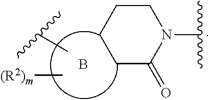

In some embodiments, Ring A is

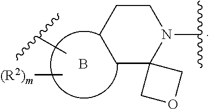

In some embodiments, Ring A is

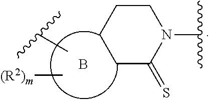

In some embodiments, Ring A is

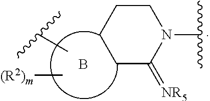

In some embodiments, Ring A is

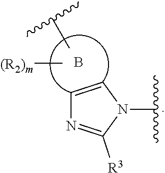

In some embodiments, Ring A is

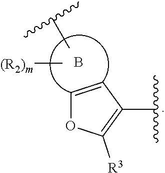

In some embodiments, Ring A is

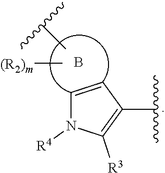

In some embodiments, Ring A is

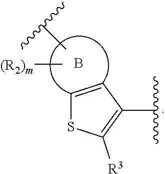

In some embodiments, Ring A is

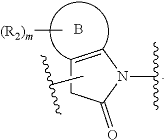

In some embodiments, Ring A is

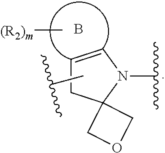

In some embodiments, Ring A is

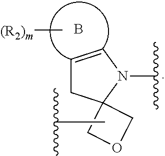

In some embodiments, Ring A is

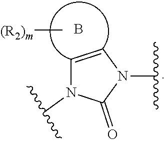

In some embodiments, Ring A is

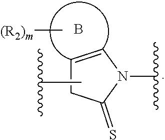

In some embodiments, Ring A is

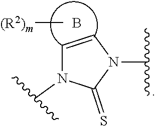

In some embodiments, Ring A is

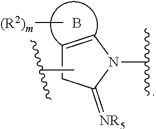

In some embodiments, Ring A is

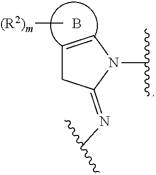

In some embodiments, Ring A is

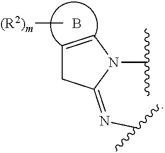

In some embodiments, Ring A is

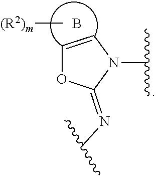

In some embodiments, Ring A is

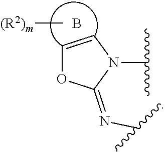

In some embodiments, Ring A is

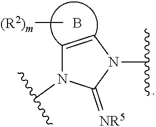

In some embodiments, Ring A is

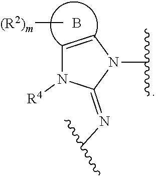

In some embodiments, Ring A is

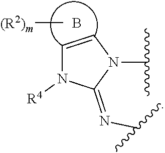

In some embodiments, Ring A is

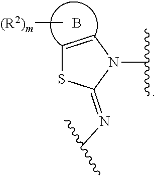

In some embodiments, Ring A is

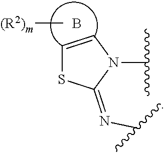

In some embodiments, Ring A is

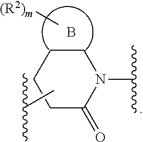

In some embodiments, Ring A is

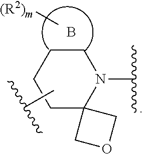

In some embodiments, Ring A is

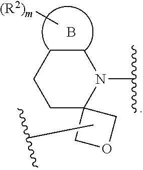

In some embodiments, Ring A is

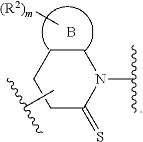

In some embodiments, Ring A is

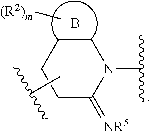

In some embodiments, Ring A is

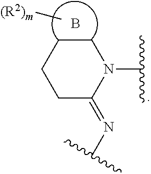

In some embodiments, Ring A is

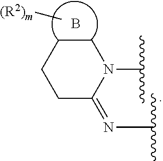

In some embodiments, Ring A is

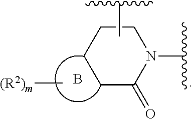

In some embodiments, Ring A is

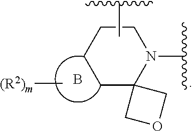

In some embodiments, Ring A is

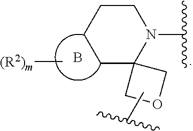

In some embodiments, Ring A is

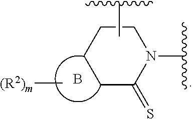

In some embodiments, Ring A is

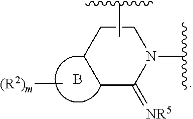

In some embodiments, Ring A is

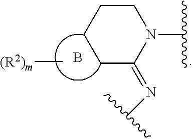

In some embodiments, Ring A is

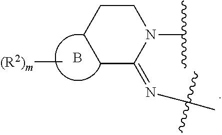

In some embodiments, Ring A is

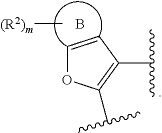

In some embodiments, Ring A is

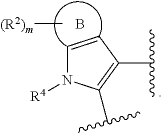

In some embodiments, Ring A is

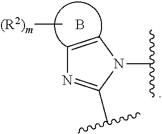

In some embodiments, Ring A is

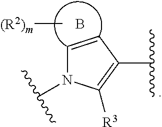

In some embodiments, Ring A is

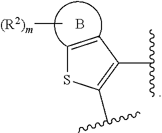

In some embodiments, Ring A is selected from those depicted in Table 1, below.

As defined above and described herein, Ring B is a fused ring selected from benzo, 5-6 membered heteroaryl containing 1-4 heteroatoms independently selected from nitrogen, oxygen, or sulfur, and a 5 to 7-membered saturated or partially unsaturated carbocyclyl or heterocyclyl with 1-3 heteroatoms independently selected from nitrogen, oxygen, or sulfur.

In some embodiments, Ring B is benzo. In some embodiments, Ring B is 5-6 membered heteroaryl containing 1-4 heteroatoms independently selected from nitrogen, oxygen, or sulfur. In some embodiments, Ring B is a 5 to 7-membered saturated or partially unsaturated carbocyclyl. In some embodiments, Ring B is 5 to 7-membered saturated or partially unsaturated heterocyclyl with 1-3 heteroatoms independently selected from boron, nitrogen, oxygen, silicon, or sulfur.

In some embodiments, Ring B is selected from those depicted in Table 1, below.

As defined above and described herein, R³ is selected from hydrogen, halogen, —OR, —NR₂, or —SR.

In some embodiments, R³ is hydrogen, halogen. In some embodiments, R³ is —OR. In some embodiments, R³ is —NR₂. In some embodiments, R³ is —SR.

In some embodiments, R³ is selected from those depicted in Table 1, below.

As defined above and described herein, each R⁴ is independently hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R.

In some embodiments, R⁴ is hydrogen. In some embodiments, R⁴ is R^{A}. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)NROR. In some embodiments, R⁴ is —OC(O)R. In some embodiments, R⁴ is —OC(O)NR₂. In some embodiments, R⁴ is —NRC(O)OR. In some embodiments, R⁴ is —NRC(O)R. In some embodiments, R⁴ is —NRC(O)NR₂. In some embodiments, R⁴ is —NRS(O)₂R.

In some embodiments, R⁴ is selected from those depicted in Table 1, below.

As defined above and described herein, R⁵ is hydrogen, C₁₋₄ aliphatic, or —CN.

In some embodiments, R⁵ is hydrogen. In some embodiments, R is C₁₋₄ aliphatic. In some embodiments, R⁵ is —CN.

In some embodiments, R⁵ is selected from those depicted in Table 1, below.

As defined above and described herein, R⁶ is hydrogen or R^{A}.

In some embodiments, R⁶ is hydrogen. In some embodiments, R⁶ is R^{A}. In some embodiments, R⁶ is ethyl. In some embodiments, R⁶ is isopropyl. In some embodiments, R⁶ is neopropyl. In some embodiments, R⁶ is tert-butyl. In some embodiments, R⁶ is cyclopropyl. In some embodiments, R⁶ is cyclobutyl. In some embodiments, R⁶ is cyclopentyl. In some embodiments, R⁶ is cyclohexyl.

In some embodiments, R⁶ is selected from those depicted in Table 1, below.

As defined above and described herein, R⁷ is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R.

In some embodiments, R⁷ is hydrogen. In some embodiments, R⁷ is R^{A}. In some embodiments, R⁷ is halogen. In some embodiments, R⁷ is —CN. In some embodiments, R⁷ is —NO₂. In some embodiments, R⁷ is —OR. In some embodiments, R⁷ is —SR. In some embodiments, R⁷ is —NR₂. In some embodiments, R⁷ is —S(O)₂R. In some embodiments, R⁷ is —S(O)₂NR₂. In some embodiments, R⁷ is —S(O)R. In some embodiments, R⁷ is —C(O)R. In some embodiments, R⁷ is —C(O)OR. In some embodiments, R⁷ is —C(O)NR₂. In some embodiments, R⁷ is —C(O)NROR. In some embodiments, R⁷ is —OC(O)R. In some embodiments, R⁷ is —OC(O)NR₂. In some embodiments, R⁷ is —NRC(O)OR. In some embodiments, R⁷ is —NRC(O)R. In some embodiments, R⁷ is —NRC(O)NR₂. In some embodiments, R⁷ is —NRS(O)₂R. In some embodiments, R⁷ is

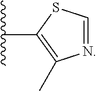 [SMILES: *c1scnc1C]

In some embodiments, R⁷ is selected from those depicted in Table 1, below.

As defined above and described herein, p is 0, 1, 2, 3, or 4.

In some embodiments, p is 0. In some embodiments, p is 1. In some embodiments, p is 2. In some embodiments, p is 3. In some embodiments, p is 4.

In some embodiments, p is selected from those depicted in Table 1, below.

As defined above and described herein, L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—.

In some embodiments, L is a covalent bond. In some embodiments, L is a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—.

In some embodiments, L is —CH₂—. In some embodiments, L is —CH₂CH₂—. In some embodiments, L is —CH₂NH—. In some embodiments, L is —CH₂CH₂CH₂—. In some embodiments, L is —CH₂CH₂CH₂CH₂—. In some embodiments, L is

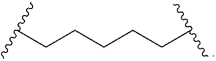 [SMILES: *CCCCC*]

In some embodiments, L is

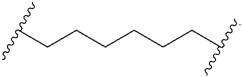 [SMILES: *CCCCCC*]

In some embodiments, L is

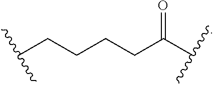 [SMILES: *CCCCC(*)=O]

In some embodiments, L is

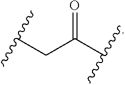 [SMILES: *CC(*)=O]

In some embodiments, L is

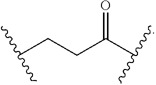 [SMILES: *CCC(*)=O]

In some embodiments, L is

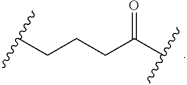 [SMILES: *CCCC(*)=O]

In some embodiments, L is

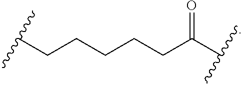 [SMILES: *CCCCCC(*)=O]

In some embodiments, L is

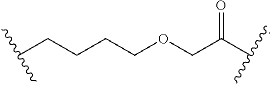 [SMILES: *CCCCOCC(*)=O]

In some embodiments, L is

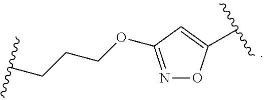 [SMILES: *CCCOc1cc(*)on1]

In some embodiments, L is

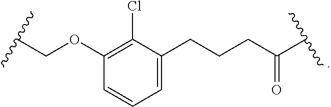 [SMILES: *COc1cccc(CCCC(*)=O)c1Cl]

In some embodiments, L is

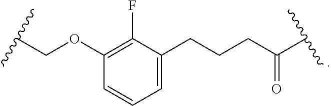 [SMILES: *COc1cccc(CCCC(*)=O)c1F]

In some embodiments, L is

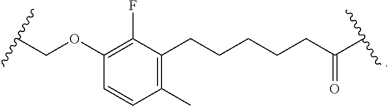 [SMILES: *COc1ccc(C)c(CCCCCC(*)=O)c1F]

In some embodiments, L is

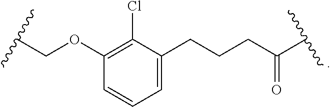 [SMILES: *COc1cccc(CCCC(*)=O)c1Cl]

In some embodiments, L is

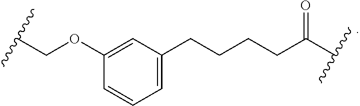 [SMILES: *COc1cccc(CCCCC(*)=O)c1]

In some embodiments, L is

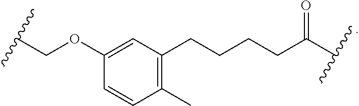 [SMILES: *COc1ccc(C)c(CCCCC(*)=O)c1]

In some embodiments, L is

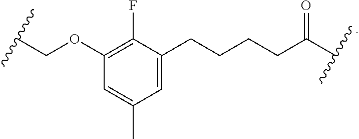 [SMILES: *COc1cc(C)cc(CCCCC(*)=O)c1F]

In some embodiments, L is

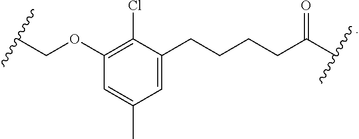 [SMILES: *COc1cc(C)cc(CCCCC(*)=O)c1Cl]

In some embodiments, L is

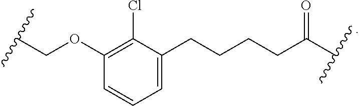 [SMILES: *COc1cccc(CCCCC(*)=O)c1Cl]

In some embodiments, L is

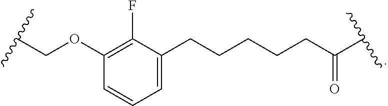 [SMILES: *COc1cccc(CCCCCC(*)=O)c1F]

In some embodiments, L is

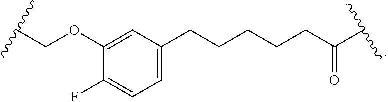 [SMILES: *COc1cc(CCCCCC(*)=O)ccc1F]

In some embodiments, L is

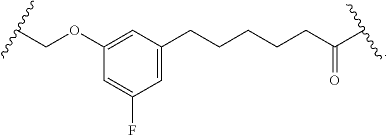 [SMILES: *COc1cc(F)cc(CCCCCC(*)=O)c1]

In some embodiments, L is

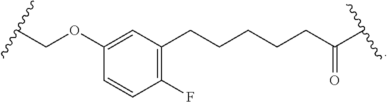 [SMILES: *COc1ccc(F)c(CCCCCC(*)=O)c1]

In some embodiments, L is

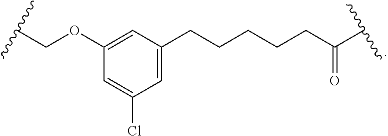 [SMILES: *COc1cc(Cl)cc(CCCCCC(*)=O)c1]

In some embodiments, L is

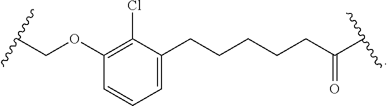 [SMILES: *COc1cccc(CCCCCC(*)=O)c1Cl]

In some embodiments, L is

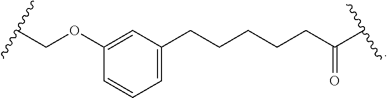 [SMILES: *COc1cccc(CCCCCC(*)=O)c1]

In some embodiments, L is

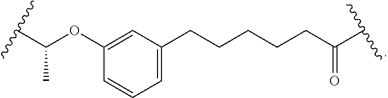 [SMILES: *C(=O)CCCCCc1cccc(O[C@@H](*)C)c1]

In some embodiments, L is

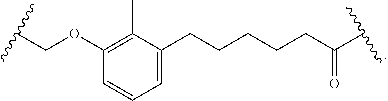 [SMILES: *COc1cccc(CCCCCC(*)=O)c1C]

In some embodiments, L is

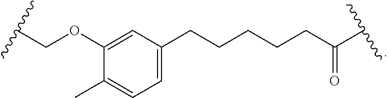 [SMILES: *COc1cc(CCCCCC(*)=O)ccc1C]

In some embodiments, L is

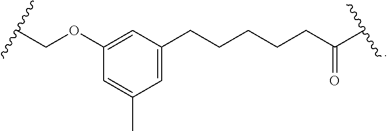 [SMILES: *COc1cc(C)cc(CCCCCC(*)=O)c1]

In some embodiments, L is

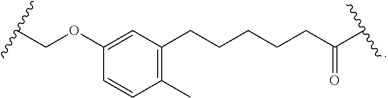 [SMILES: *COc1ccc(C)c(CCCCCC(*)=O)c1]

In some embodiments, L is

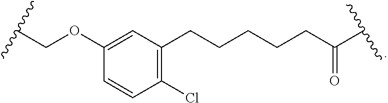 [SMILES: *COc1ccc(Cl)c(CCCCCC(*)=O)c1]

In some embodiments, L is

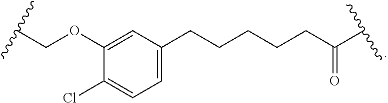 [SMILES: *COc1cc(CCCCCC(*)=O)ccc1Cl]

In some embodiments, L is

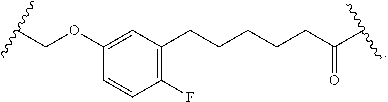 [SMILES: *COc1ccc(F)c(CCCCCC(*)=O)c1]

In some embodiments, L is

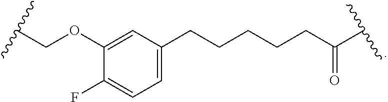 [SMILES: *COc1cc(CCCCCC(*)=O)ccc1F]

In some embodiments, L is

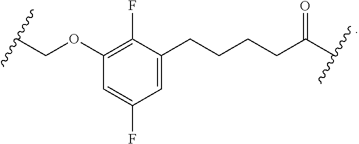 [SMILES: *COc1cc(F)cc(CCCCC(*)=O)c1F]

In some embodiments, L is

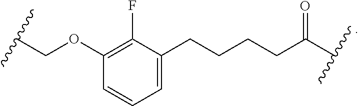 [SMILES: *COc1cccc(CCCCC(*)=O)c1F]

In some embodiments, L is

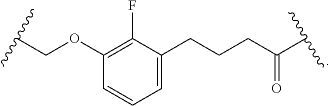 [SMILES: *COc1cccc(CCCC(*)=O)c1F]

In some embodiments, L is

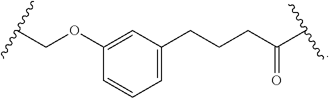 [SMILES: *COc1cccc(CCCC(*)=O)c1]

In some embodiments, L is

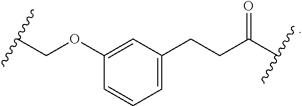 [SMILES: *COc1cccc(CCC(*)=O)c1]

In some embodiments, L is

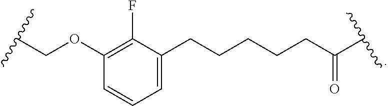 [SMILES: *COc1cccc(CCCCCC(*)=O)c1F]

In some embodiments, L is

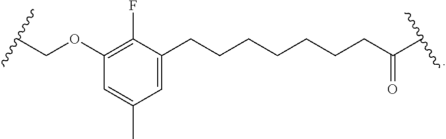 [SMILES: *COc1cc(C)cc(CCCCCCCC(*)=O)c1F]

In some embodiments, L is

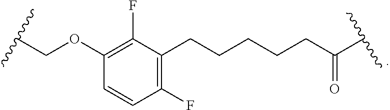 [SMILES: *COc1ccc(F)c(CCCCCC(*)=O)c1F]

In some embodiments, L is

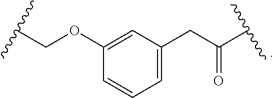 [SMILES: *COc1cccc(CC(*)=O)c1]

In some embodiments, L is

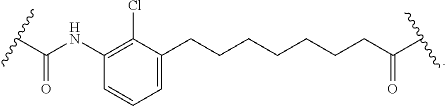 [SMILES: *C(=O)CCCCCCCc1cccc(NC(*)=O)c1Cl]

In some embodiments, L is

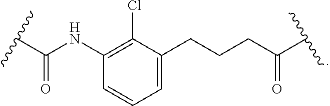 [SMILES: *C(=O)CCCc1cccc(NC(*)=O)c1Cl]

In some embodiments, L is

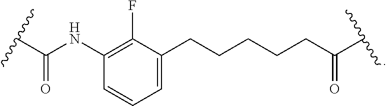 [SMILES: *C(=O)CCCCCc1cccc(NC(*)=O)c1F]

In some embodiments, L is

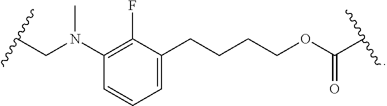 [SMILES: *CN(C)c1cccc(CCCCOC(*)=O)c1F]

In some embodiments, L is

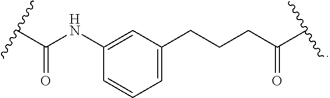 [SMILES: *C(=O)CCCc1cccc(NC(*)=O)c1]

In some embodiments, L is

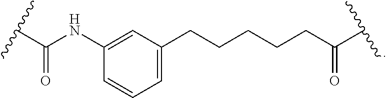 [SMILES: *C(=O)CCCCCc1cccc(NC(*)=O)c1]

In some embodiments, L is

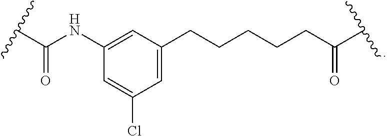 [SMILES: *C(=O)CCCCCc1cc(Cl)cc(NC(*)=O)c1]

In some embodiments, L is

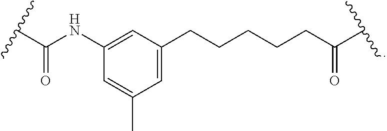 [SMILES: *C(=O)CCCCCc1cc(C)cc(NC(*)=O)c1]

In some embodiments, L is

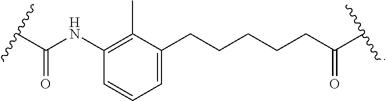 [SMILES: *C(=O)CCCCCc1cccc(NC(*)=O)c1C]

In some embodiments, L is

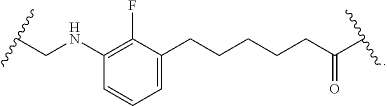 [SMILES: *CNc1cccc(CCCCCC(*)=O)c1F]

In some embodiments, L is

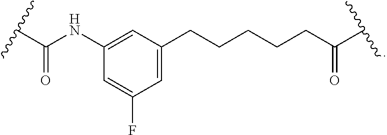 [SMILES: *C(=O)CCCCCc1cc(F)cc(NC(*)=O)c1]

In some embodiments, L is

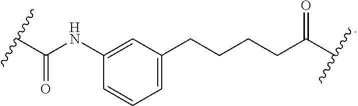 [SMILES: *C(=O)CCCCc1cccc(NC(*)=O)c1]

In some embodiments, L is

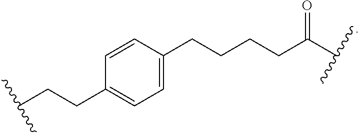 [SMILES: *CCc1ccc(CCCCC(*)=O)cc1]

In some embodiments, L is

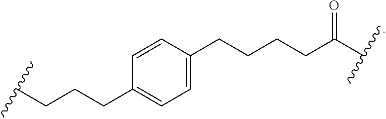 [SMILES: *CCCc1ccc(CCCCC(*)=O)cc1]

In some embodiments, L is

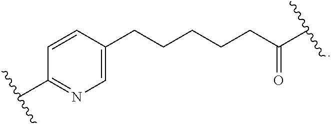 [SMILES: *C(=O)CCCCCc1ccc(*)nc1]

In some embodiments L is

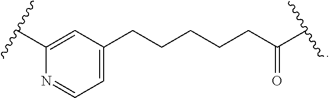 [SMILES: *C(=O)CCCCCc1ccnc(*)c1]

In some embodiments, L is

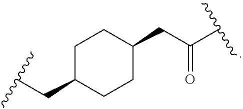 [SMILES: *C[C@H]1CC[C@@H](CC(*)=O)CC1]

In some embodiments, L is

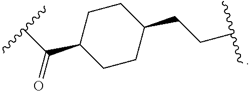 [SMILES: *CC[C@H]1CC[C@@H](C(*)=O)CC1]

In some embodiments, L is

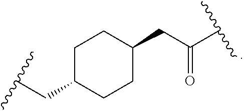 [SMILES: *C[C@H]1CC[C@H](CC(*)=O)CC1]

In some embodiments, L is

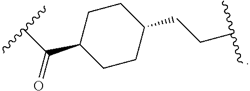 [SMILES: *CC[C@H]1CC[C@H](C(*)=O)CC1]

In some embodiments, L is

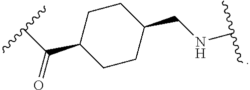 [SMILES: *NC[C@H]1CC[C@@H](C(*)=O)CC1]

In some embodiments, L is

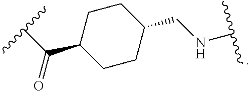 [SMILES: *NC[C@H]1CC[C@H](C(*)=O)CC1]

In some embodiments, L is

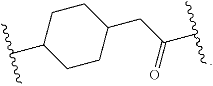 [SMILES: *C(=O)CC1CCC(*)CC1]

In some embodiments, L is

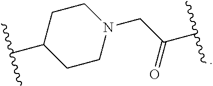 [SMILES: *C(=O)CN1CCC(*)CC1]

In some embodiments, L is selected from those depicted in Table 1, below.

Without limitation, the point of attachment of L to STAT and LBM can be, for example when L is

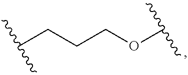 [SMILES: *CCCO*]

either

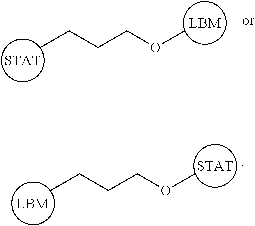

As defined above and described herein, each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

In some embodiments, -Cy- is an optionally substituted phenylenyl. In some embodiments, -Cy- is an optionally substituted 8-10 membered bicyclic arylenyl. In some embodiments, -Cy- is an optionally substituted 4-7 membered saturated or partially unsaturated carbocyclylenyl. In some embodiments, -Cy- is an optionally substituted 4-11 membered saturated or partially unsaturated spiro carbocyclylenyl. In some embodiments, -Cy- is an optionally substituted 8-10 membered bicyclic saturated or partially unsaturated carbocyclylenyl. In some embodiments, -Cy- is an optionally substituted 4-7 membered saturated or partially unsaturated heterocyclylenyl having 1-2 heteroatoms independently selected from nitrogen, oxygen, and sulfur. In some embodiments, -Cy- is an optionally substituted 4-11 membered saturated or partially unsaturated spiro heterocyclylenyl having 1-3 heteroatoms independently selected from nitrogen, oxygen, and sulfur. In some embodiments, -Cy- is an optionally substituted 8-10 membered bicyclic saturated or partially unsaturated heterocyclylenyl having 1-3 heteroatoms independently selected from nitrogen, oxygen, and sulfur. In some embodiments, -Cy- is an optionally substituted 5-6 membered heteroarylenyl having 1-4 heteroatoms independently selected from nitrogen, oxygen, and sulfur. In some embodiments, -Cy- is an optionally substituted 8-10 membered bicyclic heteroarylenyl having 1-5 heteroatoms independently selected from nitrogen, oxygen, or sulfur.

In some embodiments, -Cy- is

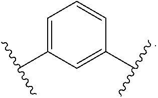 [SMILES: *c1cccc(*)c1]

In some embodiments, -Cy- is

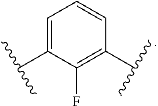 [SMILES: *c1cccc(*)c1F]

In some embodiments, -Cy- is

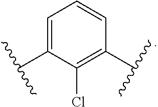 [SMILES: *c1cccc(*)c1Cl]

In some embodiments, -Cy- is

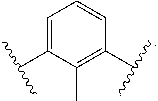 [SMILES: *c1cccc(*)c1C]

In some embodiments, -Cy- is

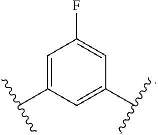 [SMILES: *c1cc(*)cc(F)c1]

In some embodiments, -Cy- is

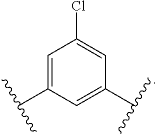 [SMILES: *c1cc(*)cc(Cl)c1]

In some embodiments, -Cy- is

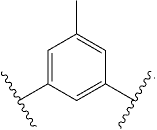 [SMILES: *c1cc(*)cc(C)c1]

In some embodiments, -Cy- is

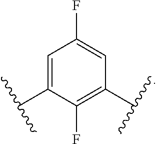 [SMILES: *c1cc(F)cc(*)c1F]

In some embodiments, -Cy- is

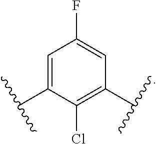 [SMILES: *c1cc(F)cc(*)c1Cl]

In some embodiments, -Cy- is

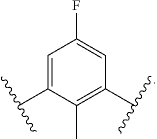 [SMILES: *c1cc(F)cc(*)c1C]

In some embodiments, -Cy- is

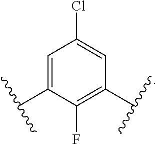 [SMILES: *c1cc(Cl)cc(*)c1F]

In some embodiments, -Cy- is

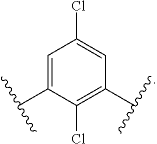 [SMILES: *c1cc(Cl)cc(*)c1Cl]

In some embodiments, -Cy- is

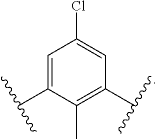 [SMILES: *c1cc(Cl)cc(*)c1C]

In some embodiments, -Cy- is

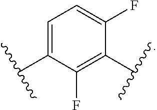 [SMILES: *c1ccc(F)c(*)c1F]

In some embodiments, -Cy- is

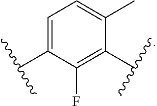 [SMILES: *c1ccc(C)c(*)c1F]

In some embodiments, -Cy- is

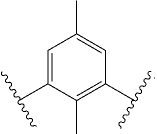 [SMILES: *c1cc(C)cc(*)c1C]

In some embodiments, -Cy- is

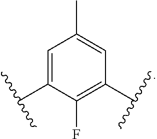 [SMILES: *c1cc(C)cc(*)c1F]

In some embodiments, -Cy- is

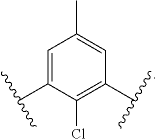 [SMILES: *c1cc(C)cc(*)c1Cl]

In some embodiments, -Cy- is

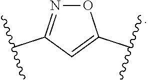 [SMILES: *c1cc(*)on1]

In some embodiments, -Cy- is selected from those depicted in Table 1, below.

As defined above and described herein, L¹ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—.

In some embodiments, L¹ is covalent bond. In some embodiments, L¹ is a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—. In some embodiments, L¹ is —C(O)—. In some embodiments, L¹ is —C(O)CH₂—. In some embodiments, L¹ is

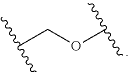 [SMILES: *CO*]

In some embodiments, L¹ is selected from those depicted in Table 1, below.

As defined above and described herein, L² is a covalent bond or a bivalent, saturated or partially unsaturated, straight or branched C₁₋₅ hydrocarbon chain, wherein 0-3 methylene units of L² are independently replaced by —O—, —NR—, —CRF—, —CF₂—, —C(O)—,

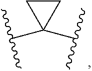 [SMILES: *C1(*)CC1]

### —S—, —S(O)—, or —S(O)₂—.

In some embodiments, L² is covalent bond. In some embodiments, L² is a bivalent, saturated or partially unsaturated, straight or branched C₁₋₅ hydrocarbon chain, wherein 0-3 methylene units of L² are independently replaced by —O—, —NR—, —CRF—, —CF₂—, —C(O)—,

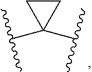 [SMILES: *C1(*)CC1]

### —S—, —S(O)—, or S(O)₂—.

In some embodiments, L² is selected from those depicted in Table 1, below.

As defined above and described herein, Q is a bivalent moiety selected from —O—, —CR₂—, —CF₂—, —CFR—, —C(O)—, —OCR₂, and —C(S)—.

In some embodiments, Q is —O—. In some embodiments, Q is —CR₂—. In some embodiments, Q is —OCR₂. In some embodiments, Q is —CF₂—. In some embodiments, Q is —CFR—. In some embodiments, Q is —C(O)—. In some embodiments, Q is —C(S)—.

In some embodiments, Q is selected from those depicted in Table 1, below.

As defined above and described herein, Y is an optionally substituted —(CH₂)_{y}—.

In some embodiments, Y is an optionally substituted —(CH₂)_{y}—. In some embodiments, Y is —CH₂—. In some embodiments, Y is

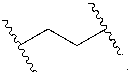 [SMILES: *CC*]

In some embodiments, Y is selected from those depicted in Table 1, below.

As defined above and described herein, y is 0, 1, 2, or 3.

In some embodiments, y is 0. In some embodiments, y is 1. In some embodiments, y is 2. In some embodiments, y is 3.

In some embodiments, y is selected from those depicted in Table 1, below.

As defined above and described herein, X is an optionally substituted —(CH₂)ₓ—.

In some embodiments, X is an optionally substituted —(CH₂)ₓ—. In some embodiments, X is

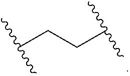 [SMILES: *CC*]

In some embodiments, X is selected from those depicted in Table 1, below.

As defined above and described herein, x is 0, 1, 2, 3, 4, or 5.

In some embodiments, x is 0. In some embodiments, x is 1. In some embodiments, x is 2. In some embodiments, x is 3. In some embodiments, x is 4. In some embodiments, x is 5.

In some embodiments, x is selected from those depicted in Table 1, below.

As defined above and described herein, R^{x} is R^{x} is hydrogen, R^{A}, —(CR₂)₁₋₃OCONR₂, or —(CR₂)₁₋₃CONR₂.

In some embodiments, R^{x} is hydrogen. In some embodiments, R^{x} is R^{A}. In some embodiments, R^{x} is —(CR₂)₁₋₃OCONR₂. In some embodiments, R^{x} is —(CR₂)₁₋₃CONR₂. In some embodiments, R^{x} is

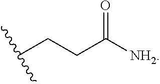 [SMILES: *CCC(N)=O]

In some embodiments, R^{x} is selected from those depicted in Table 1, below.

As defined above and described herein, R^{y1} and R^{y2} are each independently hydrogen, R^{A}, —CH₂CO₂R, or —CH₂OCO₂R.

In some embodiments, R^{y1} is hydrogen. In some embodiments, R^{y1} is R^{A}. In some embodiments, R^{y1} is —CH₂CO₂R. In some embodiments, R^{y1} is —CH₂OCO₂R. In some embodiments, R^{y2} is hydrogen. In some embodiments, R^{y2} is R^{A}. In some embodiments, R^{y2} is —CH₂CO₂R. In some embodiments, R^{y2} is —CH₂OCO₂R. In some embodiments, R^{x} is

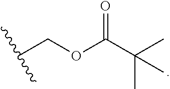 [SMILES: *COC(=O)C(C)(C)C]

In some embodiments, R^{x} is

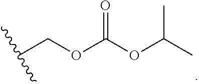 [SMILES: *COC(=O)OC(C)C]

In some embodiments, R^{y} and R^{y2} are selected from those depicted in Table 1, below.

As defined above and described herein, R^{z1} and R^{z2} are each independently hydrogen or R^{A}, or R^{z1} and R^{z2} are cyclically linked to form an optionally substituted fused 5-8 membered heterocyclic ring.

In some embodiments, R^{z1} is hydrogen. In some embodiments, R^{z1} is R^{A}. In some embodiments, R^{z2} is hydrogen. In some embodiments, R^{z2} is R^{A}. In some embodiments, R^{z1} and R² are cyclically linked to form an optionally substituted fused 5-8 membered heterocyclic ring.

In some embodiments, R^{z1} is —CH₂CH₂OH. In some embodiments, R^{z1} is ethyl. In some embodiments, R^{z1} is methyl. In some embodiments, R^{z1} is isopropyl. In some embodiments, R^{z1} is neopropyl. In some embodiments, R^{z1} is tert-butyl. In some embodiments, R^{z1} is cyclopropyl. In some embodiments, R^{z1} is cyclobutyl. In some embodiments, R^{z1} is cyclopentyl. In some embodiments, cyclohexyl. In some embodiments, R^{z1} is

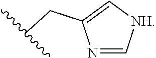 [SMILES: *Cc1c[nH]cn1]

In some embodiments, R^{z1} and R^{z2} are cyclically linked by

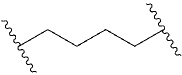 [SMILES: *CCCC*]

to form a fused 8-membered heterocyclic ring. In some embodiments, R^{z1} and R^{z2} are cyclically linked by optionally substituted —(CH₂)ₓ—, wherein 1-2 methylenes of —(CH₂)ₓ are optionally replaced with a bivalent group selected from —NR—, —N(COR)—, —N(CO₂R)—, —N(SO₂R)—, —N(CONR₂)—, and —N(SO₂NR₂)—. In some embodiments, R^{z1} and R^{z2} are cyclically linked by

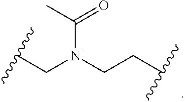 [SMILES: *CCN(C*)C(C)=O]

In some embodiments, R^{z1} and R² are cyclically linked by

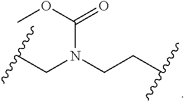 [SMILES: *CCN(C*)C(=O)OC]

In some embodiments, R^{z1} and R^{z2} are selected from those depicted in Table 1, below.

As defined above and described herein, Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;

In some embodiments, Ring C is an optionally substituted phenylenyl. In some embodiments, Ring C is an optionally substituted naphthylenyl. In some embodiments, Ring C is an optionally substituted 5-10 membered heteroarylenyl containing 1-4 heteroatoms independently selected from nitrogen, oxygen, or sulfur. In some embodiments, Ring C is an optionally substituted 5-11 membered saturated or partially unsaturated carbocyclylenyl. In some embodiments, Ring C is an optionally substituted 5-11 membered saturated or partially unsaturated heterocyclylenyl with 1-4 heteroatoms independently selected from nitrogen, oxygen, or sulfur. In some embodiments, Ring C is

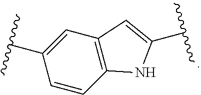 [SMILES: *c1ccc2[nH]c(*)cc2c1]

In some embodiments, Ring C is

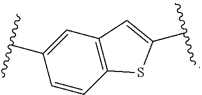 [SMILES: *c1ccc2sc(*)cc2c1]

In some embodiments, Ring C is selected from those depicted in Table 1, below.

As defined above and described herein, Ring D is selected from phenyl, a 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur, and a 5-6 membered heteroaryl with 1-4 heteroatoms independently selected from nitrogen, oxygen or sulfur.

In some embodiments, Ring D is phenyl. In some embodiments, Ring D is 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur. In some embodiments, Ring D is 5-6 membered heteroaryl with 1-4 heteroatoms independently selected from nitrogen, oxygen or sulfur.

In some embodiments, Ring D is selected from those depicted in Table 1, below.

As defined above and described herein, Ring E is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, Ring E is phenylenyl. In some embodiments, Ring E is a 4-7 membered saturated or partially unsaturated carbocyclylenyl. In some embodiments, Ring E is a heterocyclylenyl having 1-2 heteroatoms independently selected from nitrogen, oxygen, and sulfur. In some embodiments, Ring E is a 5-6 membered heteroarylenyl having 1-4 heteroatoms independently selected from nitrogen, oxygen, and sulfur. In some embodiments, Ring E is

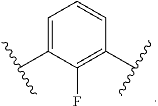 [SMILES: *c1cccc(*)c1F]

In some embodiments, Ring E is

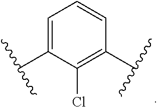 [SMILES: *c1cccc(*)c1Cl]

In some embodiments, Ring E is

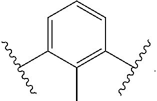 [SMILES: *c1cccc(*)c1C]

In some embodiments, Ring E is

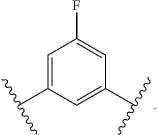 [SMILES: *c1cc(*)cc(F)c1]

In some embodiments, Ring E is

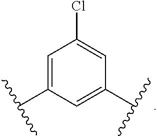 [SMILES: *c1cc(*)cc(Cl)c1]

In some embodiments, Ring E is

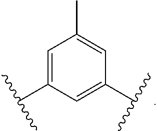 [SMILES: *c1cc(*)cc(C)c1]

In some embodiments, Ring E is

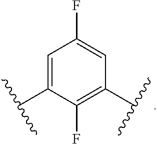 [SMILES: *c1cc(F)cc(*)c1F]

In some embodiments, Ring E is

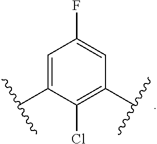 [SMILES: *c1cc(F)cc(*)c1Cl]

In some embodiments, Ring E is

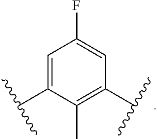 [SMILES: *c1cc(F)cc(*)c1C]

In some embodiments, Ring E is

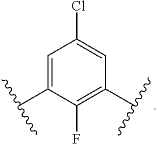 [SMILES: *c1cc(Cl)cc(*)c1F]

In some embodiments, Ring E is

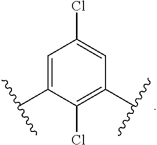 [SMILES: *c1cc(Cl)cc(*)c1Cl]

In some embodiments, Ring E is

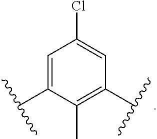 [SMILES: *c1cc(Cl)cc(*)c1C]

In some embodiments, Ring E is

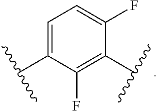 [SMILES: *c1ccc(F)c(*)c1F]

In some embodiments, Ring E is

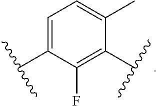 [SMILES: *c1ccc(C)c(*)c1F]

In some embodiments, Ring E is

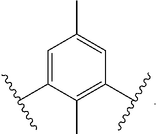 [SMILES: *c1cc(C)cc(*)c1C]

In some embodiments, Ring E is

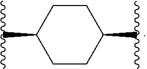 [SMILES: *[C@H]1CC[C@@H](*)CC1]

In some embodiments, Ring E is

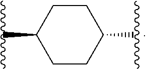 [SMILES: *[C@H]1CC[C@H](*)CC1]

In some embodiments, Ring E is selected from those depicted in Table 1, below.

As defined above and described herein, Ring F is an optionally substituted fused ring selected from a 6-membered aryl, a 5-6 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur.

In some embodiments, Ring F is an optionally substituted 6-membered aryl. In some embodiments, Ring F is an optionally substituted 5-6 membered heteroaryl containing 1-4 heteroatoms independently selected from nitrogen, oxygen, or sulfur. In some embodiments, Ring F is an optionally substituted 5-7 membered saturated or partially unsaturated carbocyclyl or heterocyclyl with 1-3 heteroatoms independently selected from nitrogen, oxygen, or sulfur. In some embodiments, Ring F is a 6-membered aryl.

In some embodiments, Ring F is selected from those depicted in Table 1, below.

As defined above and described herein, Ring G is an optionally substituted ring selected from a 5-9 membered saturated or partially unsaturated heterocyclyl.

In some embodiments, Ring G is an optionally substituted ring selected from a 5-9 membered saturated or partially unsaturated heterocyclyl.

In some embodiments, Ring G is selected from those depicted in Table 1, below.

As defined above and described herein, Ring H is absent or a ring selected from phenyl, a 5-9 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur.

In some embodiments, Ring H is absent. In some embodiments, Ring H is phenyl. In some embodiments, Ring H is a 5-9 membered heteroaryl containing 1-4 heteroatoms independently selected from nitrogen, oxygen, or sulfur. In some embodiments, Ring H is a 5-7 membered saturated or partially unsaturated carbocyclyl or heterocyclyl with 1-3 heteroatoms independently selected from nitrogen, oxygen, or sulfur.

In some embodiments, Ring H is selected from those depicted in Table 1, below.

As defined above and described herein, R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R.

In some embodiments, R^{w} is hydrogen. In some embodiments, R^{w} is R^{A}. In some embodiments, R^{w} is halogen. In some embodiments, R^{w} is —CN. In some embodiments, R^{w} is —NO₂. In some embodiments, R^{w} is —OR. In some embodiments, R^{w} is —SR. In some embodiments, R^{w} is —NR₂. In some embodiments, R^{w} is —SiR₃. In some embodiments, R^{w} is —S(O)₂R. In some embodiments, R^{w} is —S(O)₂NR₂. In some embodiments, R^{w} is —S(O)R, —C(O)R. In some embodiments, R^{w} is —C(O)OR. In some embodiments, R^{w} is —C(O)NR₂. In some embodiments, R^{w} is —C(O)NROR. In some embodiments, R^{w} is —CR₂NRC(O)R. In some embodiments, R^{w} is —CR₂NRC(O)NR₂. In some embodiments, R^{w} is —OC(O)R. In some embodiments, R^{w} is —OC(O)NR₂. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —NRC(O)OR. In some embodiments, R^{w} is —NRC(O)R. In some embodiments, R^{w} is —NRC(O)NR₂. In some embodiments, R^{w} is —NRS(O)₂R. In some embodiments, R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w} is selected from those depicted in Table 1, below.

As defined above and described herein, w is 0, 1, 2, 3 or 4.

In some embodiments, w is 0. In some embodiments, w is 1. In some embodiments, w is 2.

In some embodiments, w is 3. In some embodiments, w is 4.

In some embodiments, w is selected from those depicted in Table 1, below.

As defined above and described herein, R^{v} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R.

In some embodiments, R^{v} is hydrogen. In some embodiments, R^{v} is R^{A}. In some embodiments, R^{v} is halogen. In some embodiments, R^{v} is —CN. In some embodiments, R^{v} is —NO₂. In some embodiments, R^{v} is —OR. In some embodiments, R^{v} is —SR. In some embodiments, R^{v} is —NR₂. In some embodiments, R^{v} is —SiR₃. In some embodiments, R^{v} is —S(O)₂R. In some embodiments, R is —S(O)₂NR₂. In some embodiments, R^{v} is —S(O)R, —C(O)R. In some embodiments, R is —C(O)OR. In some embodiments, R^{v} is —C(O)NR₂. In some embodiments, R is —C(O)NROR. In some embodiments, R^{v} is —CR₂NRC(O)R. In some embodiments, R is —CR₂NRC(O)NR₂. In some embodiments, R^{v} is —OC(O)R. In some embodiments, R^{v} is —OC(O)NR₂. In some embodiments, R^{v} is —OP(O)R₂. In some embodiments, R^{v} is —OP(O)(OR)₂. In some embodiments, R^{v} is —OP(O)(OR)NR₂. In some embodiments, R^{v} is —OP(O)(NR₂)₂. In some embodiments, R^{v} is —NRC(O)OR. In some embodiments, R is —NRC(O)R. In some embodiments, R^{v} is —NRC(O)NR₂. In some embodiments, R^{v} is —NRS(O)₂R. In some embodiments, R^{v} is —NP(O)R₂. In some embodiments, R^{v} is —NRP(O)(OR)₂. In some embodiments, R^{v} is —NRP(O)(OR)NR₂. In some embodiments, R^{v} is —NRP(O)(NR₂)₂. In some embodiments, R^{v} is —NRS(O)₂R.

In some embodiments, R is selected from those depicted in Table 1, below.

As defined above and described herein, v is 0, 1, 2, 3 or 4.

In some embodiments, v is 0. In some embodiments, v is 1. In some embodiments, v is 2. In some embodiments, v is 3. In some embodiments, v is 4.

In some embodiments, v is selected from those depicted in Table 1, below.

As defined above and described herein, n is 0 or 1.

In some embodiments, n is 0. In some embodiments, n is 1.

In some embodiments, n is selected from those depicted in Table 1, below.

In some embodiments, LBM is

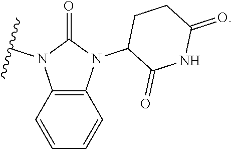 [SMILES: *n1c(=O)n(C2CCC(=O)NC2=O)c2ccccc21]

In some embodiments, LBM is

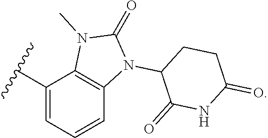 [SMILES: *c1cccc2c1n(C)c(=O)n2C1CCC(=O)NC1=O]

In some embodiments, LBM is

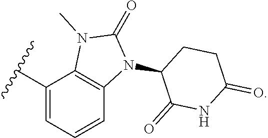 [SMILES: *c1cccc2c1n(C)c(=O)n2[C@H]1CCC(=O)NC1=O]

In some embodiments, LBM is

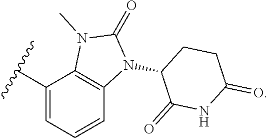 [SMILES: *c1cccc2c1n(C)c(=O)n2[C@@H]1CCC(=O)NC1=O]

In some embodiments, LBM is

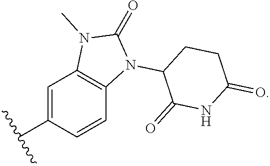 [SMILES: *c1ccc2c(c1)n(C)c(=O)n2C1CCC(=O)NC1=O]

In some embodiments, LBM is

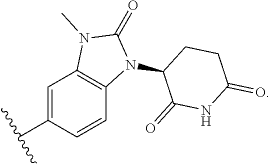 [SMILES: *c1ccc2c(c1)n(C)c(=O)n2[C@H]1CCC(=O)NC1=O]

In some embodiments, LBM is

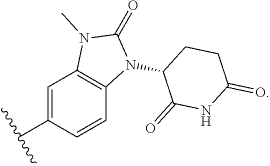 [SMILES: *c1ccc2c(c1)n(C)c(=O)n2[C@@H]1CCC(=O)NC1=O]

In some embodiments, LBM is

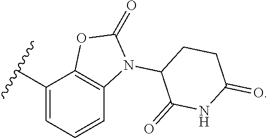 [SMILES: *c1cccc2c1oc(=O)n2C1CCC(=O)NC1=O]

In some embodiments, LBM is

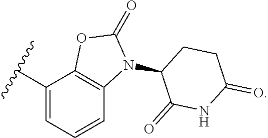 [SMILES: *c1cccc2c1oc(=O)n2[C@H]1CCC(=O)NC1=O]

In some embodiments, LBM is

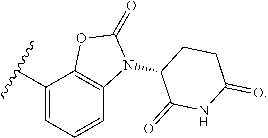 [SMILES: *c1cccc2c1oc(=O)n2[C@@H]1CCC(=O)NC1=O]

In some embodiments, LBM is

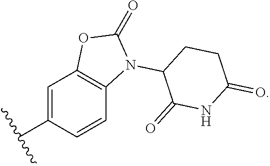 [SMILES: *c1ccc2c(c1)oc(=O)n2C1CCC(=O)NC1=O]

In some embodiments, LBM is

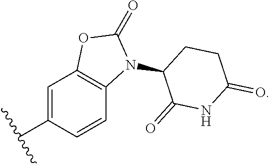 [SMILES: *c1ccc2c(c1)oc(=O)n2[C@H]1CCC(=O)NC1=O]

In some embodiments, LBM is

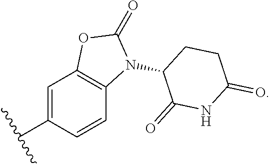 [SMILES: *c1ccc2c(c1)oc(=O)n2[C@@H]1CCC(=O)NC1=O]

In some embodiments, LBM is

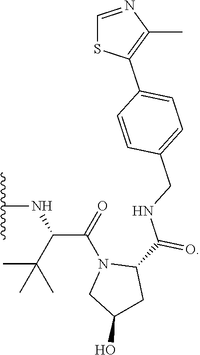 [SMILES: *N[C@H](C(=O)N1C[C@H](O)C[C@H]1C(=O)NCc1ccc(-c2scnc2C)cc1)C(C)(C)C]

In some embodiments, LBM is

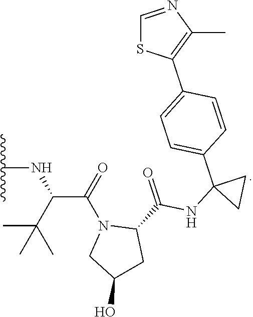 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

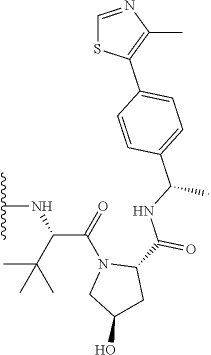 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

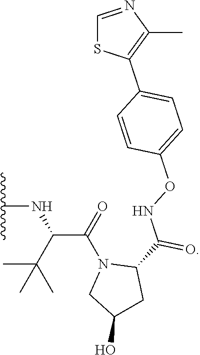 [SMILES: *N[C@H](C(=O)N1C[C@H](O)C[C@H]1C(=O)NOc1ccc(-c2scnc2C)cc1)C(C)(C)C]

In some embodiments, LBM is

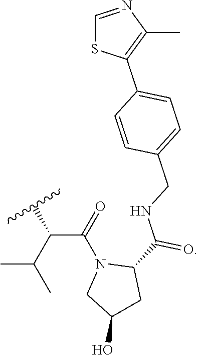 [SMILES: *[C@H](C(=O)N1C[C@H](O)C[C@H]1C(=O)NCc1ccc(-c2scnc2C)cc1)C(C)C]

As described above, in another aspect, the present invention provides a compound of formula I-g:

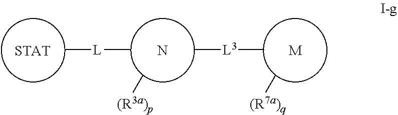

or a pharmaceutically acceptable salt thereof, wherein: Ring M is selected from

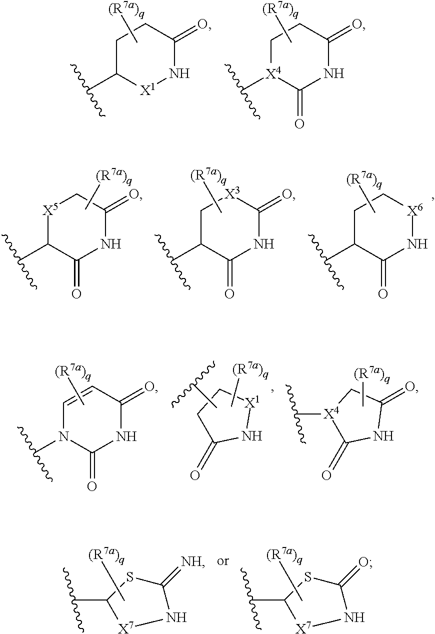

X¹, X⁶, and X⁷ are independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

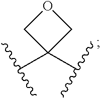 [SMILES: *C1(*)COC1]

X³ and X⁵ are independently a bivalent moiety selected from a covalent bond, —CR₂—, —NR—, —O—, —S—, or X⁴ is a trivalent moiety selected from

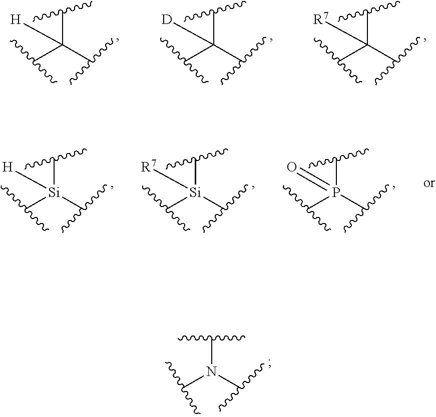

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{7a} is independently hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
   R^{7a} and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
   two R^{7a} groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
   two R^{7a} groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring N is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L³ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
p is 0, 1, 2, 3, or 4;
q is 0, 1, 2, 3, or 4; and
STAT is a STAT3 binding moiety.

As defined above and described herein, X¹, X⁶, and X⁷ are independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

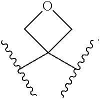 [SMILES: *C1(*)COC1]

In some embodiments, one or more of X¹, X⁶, and X⁷ is a covalent bond. In some embodiments, one or more of X¹, X⁶, and X⁷ is —CH₂—. In some embodiments, one or more of X¹, X⁶, and X⁷ is —CR₂—. In some embodiments, one or more of X¹, X⁶, and X⁷ is —C(O)—. In some embodiments, one or more of X¹, X⁶, and X⁷ is —C(S)—. In some embodiments, one or more of X¹, X⁶, and X⁷ is —CH(R)—. In some embodiments, one or more of X¹, X⁶, and X⁷ is —CH(CF₃)—. In some embodiments, one or more of X¹, X⁶, and X⁷ is —P(O)(OR)—. In some embodiments, one or more of X¹, X⁶, and X⁷ is —P(O)(R)—. In some embodiments, one or more of X¹, X⁶, and X⁷ is —P(O)NR₂—. In some embodiments, one or more of X¹, X⁶, and X⁷ is —S(O)—. In some embodiments, one or more of X¹, X⁶, and X⁷ is —S(O)₂—. In some embodiments, one or more of X¹, X⁶, and X⁷ is

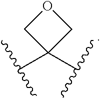 [SMILES: *C1(*)COC1]

In some embodiments, X¹, X⁶, and X⁷ are independently selected from those depicted in Table 1 below.

As defined above and described herein, X² is a carbon atom, nitrogen atom, or silicon atom.

In some embodiments, X² is a carbon atom. In some embodiments, X² is a nitrogen atom. In some embodiments, X² is a silicon atom.

In some embodiments, X² is selected from those depicted in Table 1 below.

As defined above and described herein, X³ and X⁵ are independently a bivalent moiety selected from —CH₂—, —CR₂—, —NR—, —CF₂—, —CHF—, —S—, —CH(R)—, —SiR₂—, or —O—.

In some embodiments, one or more of X³ and X⁵ is —CH₂—. In some embodiments, one or more of X³ and X⁵ is —CR₂—. In some embodiments, one or more of X³ and X⁵ is —NR—. In some embodiments, one or more of X³ and X⁵ is —CF₂—. In some embodiments, one or more of X³ and X⁵ is —CHF—. In some embodiments, one or more of X³ and X⁵ is —S—. In some embodiments, one or more of X³ and X⁵ is —CH(R)—. In some embodiments, one or more of X³ and X⁵ is —SiR₂—. In some embodiments, one or more of X³ and X⁵ is —O—.

In some embodiments, X³ and X⁵ are independently selected from those depicted in Table 1 below.

As defined above and described herein, X⁴ is a trivalent moiety selected from

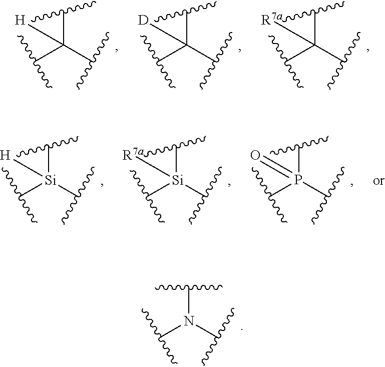

In some embodiments, X⁴ is

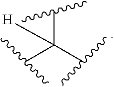 [SMILES: *C(*)*]

In some embodiments, X⁴ is

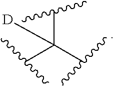 [SMILES: *C(*)(*)[2H]]

In some embodiments, X⁴ is

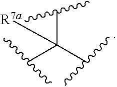

In some embodiments, X⁴ is

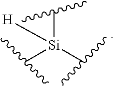 [SMILES: *[SiH](*)*]

In some embodiments, X⁴ is

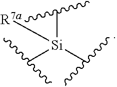

In some embodiments, X⁴ is

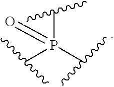 [SMILES: *P(*)(*)=O]

In some embodiments, X⁴ is

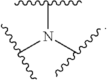 [SMILES: *N(*)*]

In some embodiments, X⁴ is selected from those depicted in Table 1 below.

As defined above and described herein, each R^{3a} is independently hydrogen, deuterium, R^{A}, halogen, —CN, —NO₂, —OR, —Si(OH)₂R, —Si(OH)R₂, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R^{3a} is hydrogen. In some embodiments, R^{3a} is deuterium. In some embodiments, R^{3a} is R^{A}. In some embodiments, R^{3a} is halogen. In some embodiments, R^{3a} is —CN. In some embodiments, R^{3a} is —NO₂. In some embodiments, R^{3a} is —OR. In some embodiments, R^{3a} is —Si(OH)₂R. In some embodiments, R^{3a} is —Si(OH)R₂. In some embodiments, R^{3a} is —SR. In some embodiments, R^{3a} is —NR₂. In some embodiments, R^{3a} is —SiR₃. In some embodiments, R^{3a} is —S(O)₂R. In some embodiments, R^{3a} is —S(O)₂NR₂. In some embodiments, R^{3a} is —S(O)R. In some embodiments, R^{3a} is —C(O)R. In some embodiments, R^{3a} is —C(O)OR. In some embodiments, R^{3a} is —C(O)NR₂. In some embodiments, R^{3a} is —C(O)N(R)OR. In some embodiments, R^{3a} is —C(R)₂N(R)C(O)R. In some embodiments, R^{3a} is —C(R)₂N(R)C(O)NR₂. In some embodiments, R^{3a} is —OC(O)R. In some embodiments, R^{3a} is —OC(O)NR₂. In some embodiments, R^{3a} is —OP(O)R₂. In some embodiments, R^{3a} is —OP(O)(OR)₂. In some embodiments, R^{3a} is —OP(O)(OR)NR₂. In some embodiments, R^{3a} is —OP(O)(NR₂)₂—. In some embodiments, R^{3a} is —N(R)C(O)OR. In some embodiments, R^{3a} is —N(R)C(O)R. In some embodiments, R^{3a} is —N(R)C(O)NR₂. In some embodiments, R^{3a} is —NP(O)R₂. In some embodiments, R^{3a} is —N(R)P(O)(OR)₂. In some embodiments, R^{3a} is —N(R)P(O)(OR)NR₂. In some embodiments, R^{3a} is —N(R)P(O)(NR₂)₂. In some embodiments, R^{3a} is —N(R)S(O)₂R.

In some embodiments, R^{3a} is selected from those depicted in Table 1 below.

As defined generally above, each R^{7a} is independently hydrogen, deuterium, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R^{7a} and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur, or two R^{7a} groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur, or two R^{7a} groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R^{7a} is hydrogen. In some embodiments, R^{7a} is deuterium. In some embodiments, R^{7a} is halogen. In some embodiments, R^{7a} is —CN. In some embodiments, R^{7a} is —OR. In some embodiments, R^{7a} is —SR. In some embodiments, R^{7a} is —S(O)R. In some embodiments, R^{7a} is —S(O)₂R. In some embodiments, R^{7a} is —NR₂. In some embodiments, R^{7a} is —Si(R)₃. In some embodiments, R^{7a} is —P(O)(R)₂. In some embodiments, R^{7a} is —P(O)(OR)₂. In some embodiments, R^{7a} is —P(O)(NR₂)OR. In some embodiments, R^{7a} is —P(O)(NR₂)₂. In some embodiments, R^{7a} is —Si(OH)R₂. In some embodiments, R^{7a} is —Si(OH)₂R. In some embodiments, R^{7a} is an optionally substituted C₁₋₄ aliphatic. In some embodiments, R^{7a} and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur. In some embodiments, two R^{7a} groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur. In some embodiments, two R^{7a} groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur. In some embodiments, two R^{7a} groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R^{7a} is selected from hydrogen, halogen, —CN, —OR, —NR₂, or C₁₋₄ alkyl. In some embodiments, R^{7a} is selected from hydrogen, halogen, —CN, or C₁₋₄ alkyl. In some embodiments, R⁷ is fluoro. In some embodiments, two R^{7a} groups on the same carbon are optionally taken together with their intervening atoms to form a 3- or 4-membered spiro fused ring.

In some embodiments, R⁷ is selected from those depicted in Table 1 below.

As defined above and described herein, Ring N is a ring selected from 6 to 10-membered aryl or heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;

In some embodiments, Ring N is a 6 to 10-membered aryl. In some embodiments, Ring N is a 6 to 10-membered heteroaryl containing 1-4 heteroatoms independently selected from nitrogen, oxygen, or sulfur. In some embodiments, Ring N is a 5 to 7-membered saturated or partially unsaturated carbocyclyl. In some embodiments, Ring N is 5 to 7-membered saturated or partially saturated heterocyclyl with 1-3 heteroatoms independently selected from boron, nitrogen, oxygen, silicon, or sulfur. In some embodiments, Ring N is 5-membered heteroaryl with 1-4 heteroatoms independently selected from boron, nitrogen, oxygen, silicon, or sulfur.

In some embodiments, Ring N is isoquinoline. In some embodiments, Ring N is imidazo[1,2-a]pyridine.

In some embodiments, Ring N is selected from those depicted in Table 1 below.

As defined above and described herein, Ring M is selected from

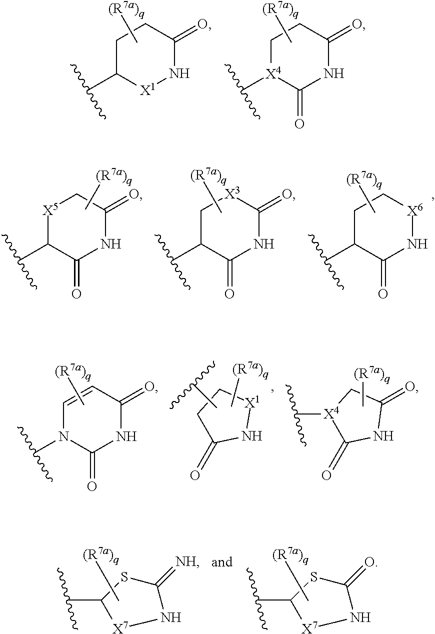

In some embodiments, Ring M is

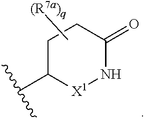

In some embodiments, Ring M is

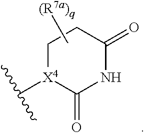

In some embodiments, Ring M is

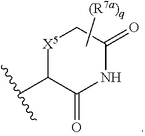

In some embodiments, Ring M is

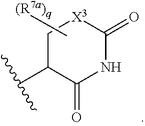

In some embodiments, Ring M is

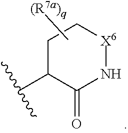

In some embodiments, Ring M is

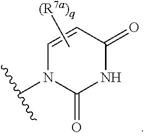

In some embodiments, Ring M is

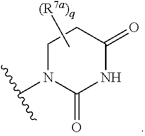

In some embodiments, Ring M is

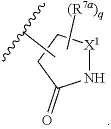

In some embodiments, Ring M is

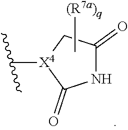

In some embodiments, Ring M is

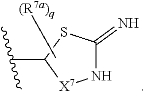

In some embodiments, Ring M is

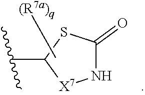

In some embodiments, Ring M is selected from those depicted in Table 1 below.

As defined above and described here, L³ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;

In some embodiments, L³ is a covalent bond. In some embodiments, L³ is a C₁₋₃ aliphatic. In some embodiments, L³ is —CH₂—. In some embodiments, L³ is —C(D)(H)—. In some embodiments, L³ is —C(D)₂-. In some embodiments, L³ is —CH₂CH₂—. In some embodiments, L¹ is —NR—. In some embodiments, L³ is —CH₂NR—. In some embodiments, L³ is or —O—. In some embodiments, L³ is —CH₂O—. In some embodiments, L³ is —S—. In some embodiments, L³ is —OC(O)—. In some embodiments, L³ is —C(O)O—. In some embodiments, L³ is —C(O)—. In some embodiments, L³ is —S(O)—. In some embodiments, L³ is —S(O)₂—. In some embodiments, L³ is —NRS(O)₂—. In some embodiments, L³ is —S(O)₂NR—. In some embodiments, L³ is —NRC(O)—. In some embodiments, L³ is —C(O)NR—.

In some embodiments, L³ is selected from those depicted in Table 1 below.

As defined above and described herein, p is 0, 1, 2, 3 or 4.

In some embodiments, p is 0. In some embodiments, p is 1. In some embodiments, p is 2. In some embodiments, p is 3. In some embodiments, p is 4.

In some embodiments, p is selected from those depicted in Table 1 below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiment, the present invention provides a compound of formula I-g, wherein the STAT3 binding moiety is the STAT3 binding moiety of formula I-a to I-f, e.g,

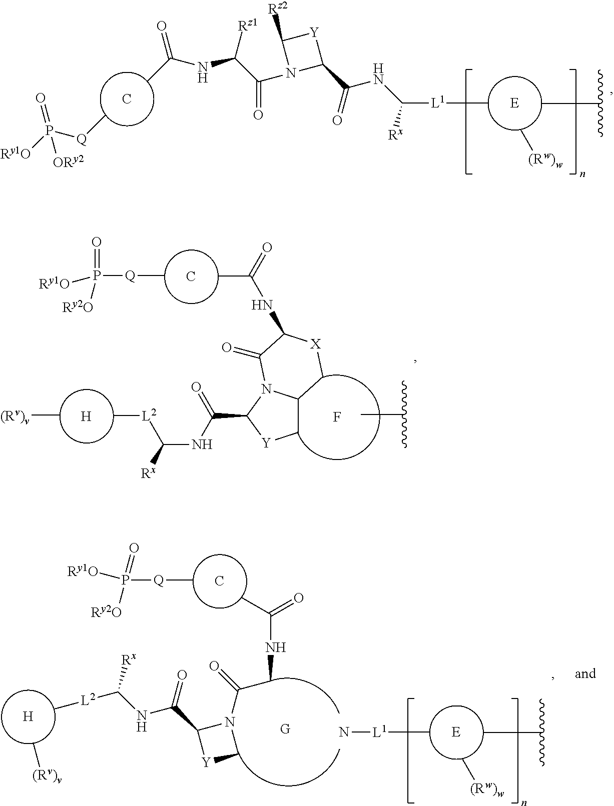

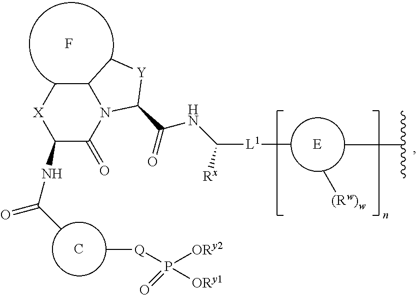

or a pharmaceutically acceptable salt thereof, wherein each variable is as defined and described herein.

In some embodiments, LBM is

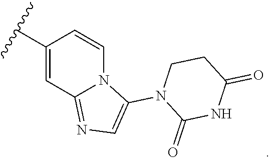 [SMILES: *c1ccn2c(N3CCC(=O)NC3=O)cnc2c1]

In some embodiments, LBM is

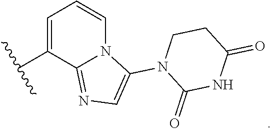 [SMILES: *c1cccn2c(N3CCC(=O)NC3=O)cnc12]

In some embodiments LBM is

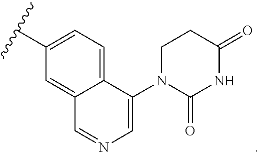 [SMILES: *c1ccc2c(N3CCC(=O)NC3=O)cncc2c1]

In some embodiments, LBM is

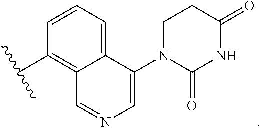 [SMILES: *c1cccc2c(N3CCC(=O)NC3=O)cncc12]

In some embodiments, STAT is

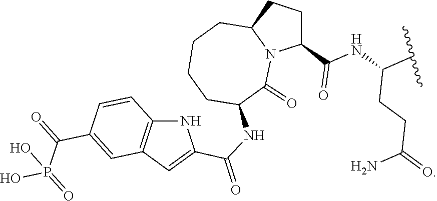 [SMILES: *[C@H](CCC(N)=O)NC(=O)[C@@H]1CC[C@@H]2CCCC[C@H](NC(=O)c3cc4cc(C(=O)P(=O)(O)O)ccc4[nH]3)C(=O)N21]

In some embodiments, STAT is

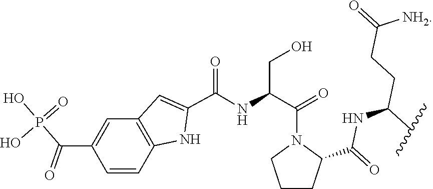 [SMILES: *[C@H](CCC(N)=O)NC(=O)[C@@H]1CCCN1C(=O)[C@H](CO)NC(=O)c1cc2cc(C(=O)P(=O)(O)O)ccc2[nH]1]

In some embodiments, STAT is

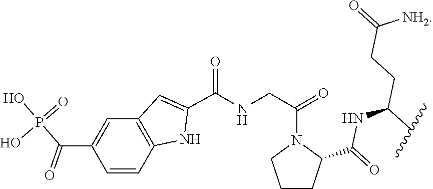 [SMILES: *[C@H](CCC(N)=O)NC(=O)[C@@H]1CCCN1C(=O)CNC(=O)c1cc2cc(C(=O)P(=O)(O)O)ccc2[nH]1]

In some embodiments, STAT is

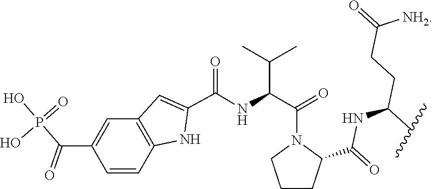 [SMILES: *[C@H](CCC(N)=O)NC(=O)[C@@H]1CCCN1C(=O)[C@@H](NC(=O)c1cc2cc(C(=O)P(=O)(O)O)ccc2[nH]1)C(C)C]

In some embodiments, STAT is

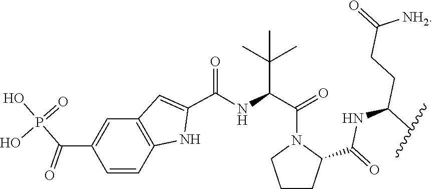 [SMILES: *[C@H](CCC(N)=O)NC(=O)[C@@H]1CCCN1C(=O)[C@@H](NC(=O)c1cc2cc(C(=O)P(=O)(O)O)ccc2[nH]1)C(C)(C)C]

In some embodiments, STAT is

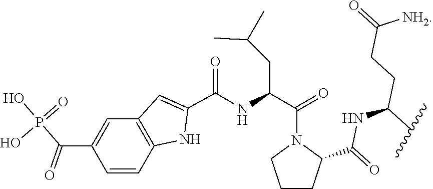 [SMILES: *[C@H](CCC(N)=O)NC(=O)[C@@H]1CCCN1C(=O)[C@H](CC(C)C)NC(=O)c1cc2cc(C(=O)P(=O)(O)O)ccc2[nH]1]

In some embodiments, STAT is

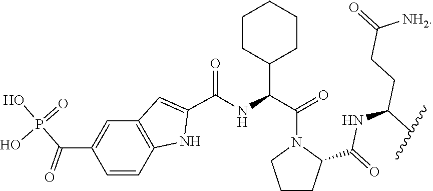 [SMILES: *[C@H](CCC(N)=O)NC(=O)[C@@H]1CCCN1C(=O)[C@@H](NC(=O)c1cc2cc(C(=O)P(=O)(O)O)ccc2[nH]1)C1CCCCC1]

In some embodiments, STAT is

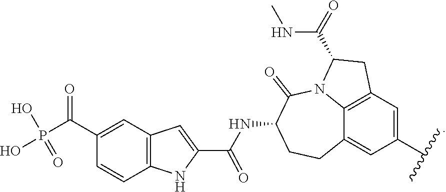 [SMILES: *c1cc2c3c(c1)C[C@@H](C(=O)NC)N3C(=O)[C@@H](NC(=O)c1cc3cc(C(=O)P(=O)(O)O)ccc3[nH]1)CC2]

In some embodiments, STAT is

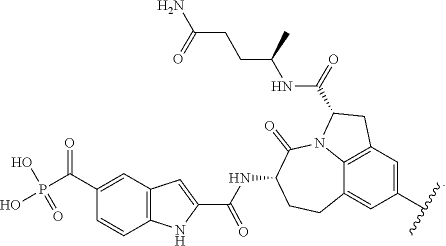 [SMILES: *c1cc2c3c(c1)C[C@@H](C(=O)N[C@H](C)CCC(N)=O)N3C(=O)[C@@H](NC(=O)c1cc3cc(C(=O)P(=O)(O)O)ccc3[nH]1)CC2]

In some embodiments, STAT is

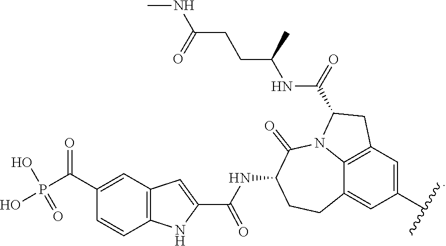 [SMILES: *c1cc2c3c(c1)C[C@@H](C(=O)N[C@H](C)CCC(=O)NC)N3C(=O)[C@@H](NC(=O)c1cc3cc(C(=O)P(=O)(O)O)ccc3[nH]1)CC2]

In some embodiments, STAT is

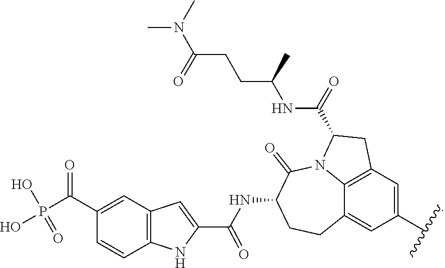 [SMILES: *c1cc2c3c(c1)C[C@@H](C(=O)N[C@H](C)CCC(=O)N(C)C)N3C(=O)[C@@H](NC(=O)c1cc3cc(C(=O)P(=O)(O)O)ccc3[nH]1)CC2]

In some embodiments, STAT is

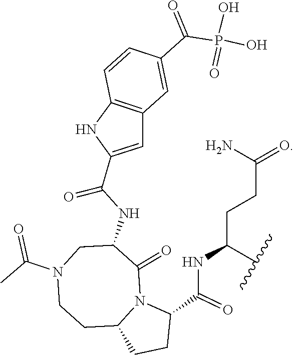 [SMILES: *[C@H](CCC(N)=O)NC(=O)[C@@H]1CC[C@@H]2CCN(C(C)=O)C[C@H](NC(=O)c3cc4cc(C(=O)P(=O)(O)O)ccc4[nH]3)C(=O)N21]

In some embodiments, STAT is

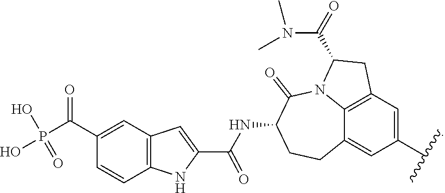 [SMILES: *c1cc2c3c(c1)C[C@@H](C(=O)N(C)C)N3C(=O)[C@@H](NC(=O)c1cc3cc(C(=O)P(=O)(O)O)ccc3[nH]1)CC2]

In some embodiments, STAT is

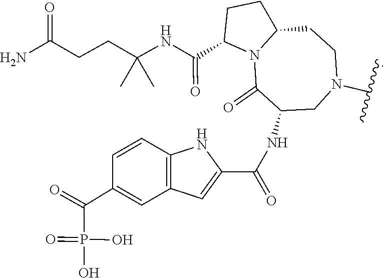 [SMILES: *N1CC[C@H]2CC[C@@H](C(=O)NC(C)(C)CCC(N)=O)N2C(=O)[C@@H](NC(=O)c2cc3cc(C(=O)P(=O)(O)O)ccc3[nH]2)C1]

In some embodiments, STAT is

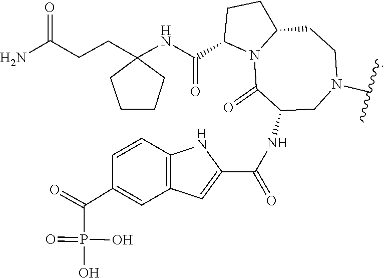 [SMILES: *N1CC[C@H]2CC[C@@H](C(=O)NC3(CCC(N)=O)CCCC3)N2C(=O)[C@@H](NC(=O)c2cc3cc(C(=O)P(=O)(O)O)ccc3[nH]2)C1]

In some embodiments, STAT is

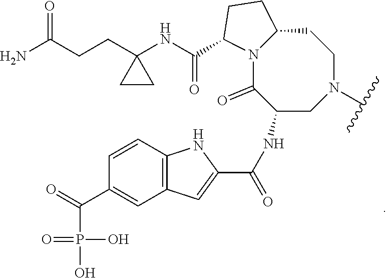 [SMILES: *N1CC[C@H]2CC[C@@H](C(=O)NC3(CCC(N)=O)CC3)N2C(=O)[C@@H](NC(=O)c2cc3cc(C(=O)P(=O)(O)O)ccc3[nH]2)C1]

In some embodiments, STAT is

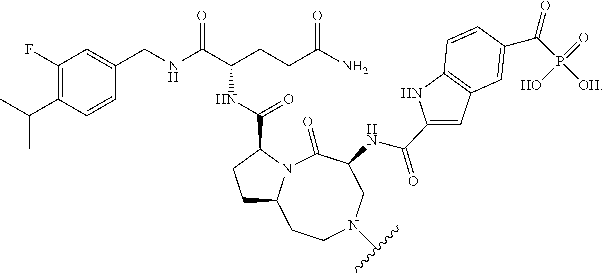 [SMILES: *N1CC[C@H]2CC[C@@H](C(=O)N[C@@H](CCC(N)=O)C(=O)NCc3ccc(C(C)C)c(F)c3)N2C(=O)[C@@H](NC(=O)c2cc3cc(C(=O)P(=O)(O)O)ccc3[nH]2)C1]

In some embodiments, STAT is

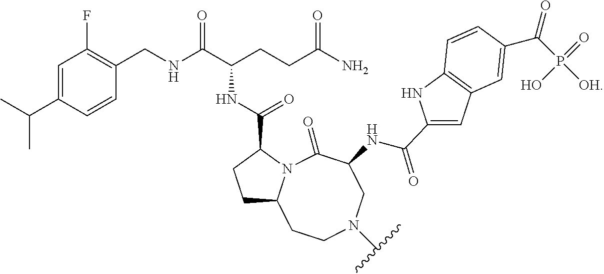 [SMILES: *N1CC[C@H]2CC[C@@H](C(=O)N[C@@H](CCC(N)=O)C(=O)NCc3ccc(C(C)C)cc3F)N2C(=O)[C@@H](NC(=O)c2cc3cc(C(=O)P(=O)(O)O)ccc3[nH]2)C1]

In some embodiments, STAT is

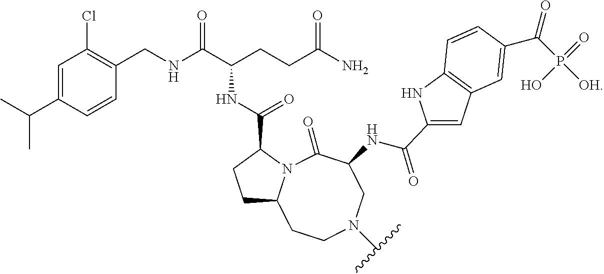 [SMILES: *N1CC[C@H]2CC[C@@H](C(=O)N[C@@H](CCC(N)=O)C(=O)NCc3ccc(C(C)C)cc3Cl)N2C(=O)[C@@H](NC(=O)c2cc3cc(C(=O)P(=O)(O)O)ccc3[nH]2)C1]

In some embodiments, STAT is

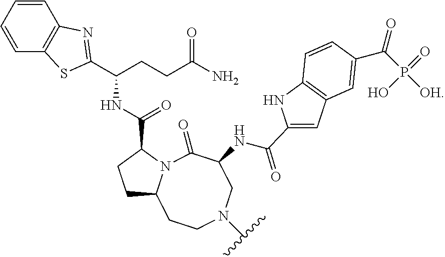 [SMILES: *N1CC[C@H]2CC[C@@H](C(=O)N[C@@H](CCC(N)=O)c3nc4ccccc4s3)N2C(=O)[C@@H](NC(=O)c2cc3cc(C(=O)P(=O)(O)O)ccc3[nH]2)C1]

In some embodiments, STAT is

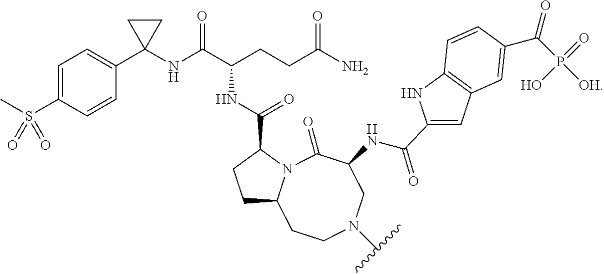 [SMILES: *N1CC[C@H]2CC[C@@H](C(=O)N[C@@H](CCC(N)=O)C(=O)NC3(c4ccc(S(C)(=O)=O)cc4)CC3)N2C(=O)[C@@H](NC(=O)c2cc3cc(C(=O)P(=O)(O)O)ccc3[nH]2)C1]

In some embodiments, STAT is

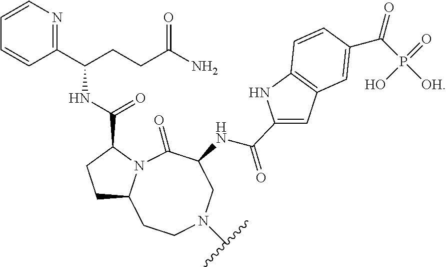 [SMILES: *N1CC[C@H]2CC[C@@H](C(=O)N[C@@H](CCC(N)=O)c3ccccn3)N2C(=O)[C@@H](NC(=O)c2cc3cc(C(=O)P(=O)(O)O)ccc3[nH]2)C1]

In some embodiments, STAT is

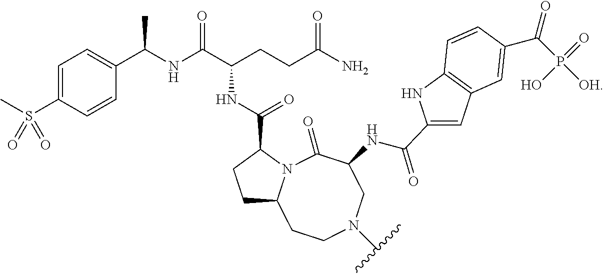 [SMILES: *N1CC[C@H]2CC[C@@H](C(=O)N[C@@H](CCC(N)=O)C(=O)N[C@H](C)c3ccc(S(C)(=O)=O)cc3)N2C(=O)[C@@H](NC(=O)c2cc3cc(C(=O)P(=O)(O)O)ccc3[nH]2)C1]

In some embodiments, STAT is

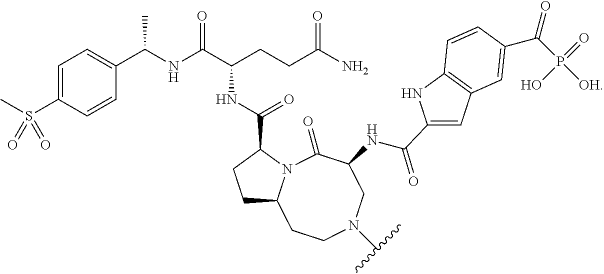 [SMILES: *N1CC[C@H]2CC[C@@H](C(=O)N[C@@H](CCC(N)=O)C(=O)N[C@@H](C)c3ccc(S(C)(=O)=O)cc3)N2C(=O)[C@@H](NC(=O)c2cc3cc(C(=O)P(=O)(O)O)ccc3[nH]2)C1]

In some embodiments, STAT is

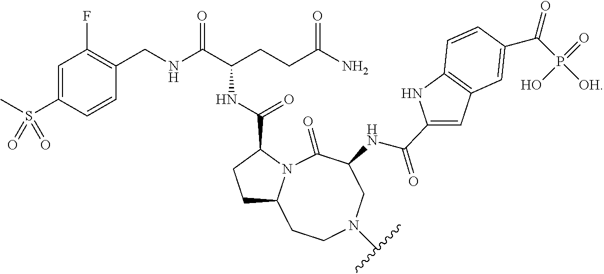 [SMILES: *N1CC[C@H]2CC[C@@H](C(=O)N[C@@H](CCC(N)=O)C(=O)NCc3ccc(S(C)(=O)=O)cc3F)N2C(=O)[C@@H](NC(=O)c2cc3cc(C(=O)P(=O)(O)O)ccc3[nH]2)C1]

In some embodiments, STAT is

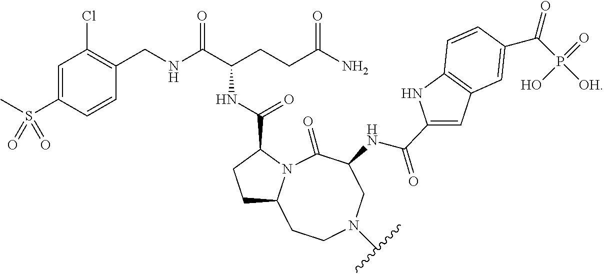 [SMILES: *N1CC[C@H]2CC[C@@H](C(=O)N[C@@H](CCC(N)=O)C(=O)NCc3ccc(S(C)(=O)=O)cc3Cl)N2C(=O)[C@@H](NC(=O)c2cc3cc(C(=O)P(=O)(O)O)ccc3[nH]2)C1]

In some embodiments, STAT is

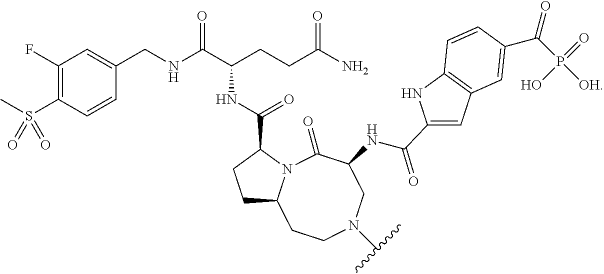 [SMILES: *N1CC[C@H]2CC[C@@H](C(=O)N[C@@H](CCC(N)=O)C(=O)NCc3ccc(S(C)(=O)=O)c(F)c3)N2C(=O)[C@@H](NC(=O)c2cc3cc(C(=O)P(=O)(O)O)ccc3[nH]2)C1]

In some embodiments, STAT is

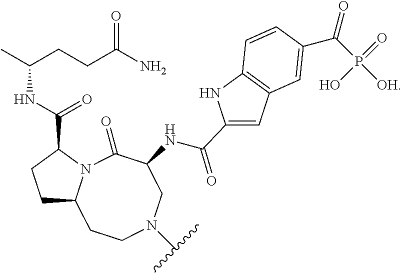 [SMILES: *N1CC[C@H]2CC[C@@H](C(=O)N[C@H](C)CCC(N)=O)N2C(=O)[C@@H](NC(=O)c2cc3cc(C(=O)P(=O)(O)O)ccc3[nH]2)C1]

In some embodiments, STAT is

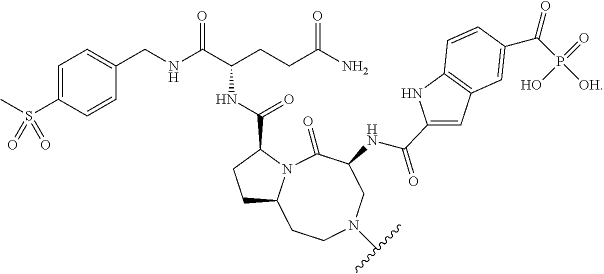 [SMILES: *N1CC[C@H]2CC[C@@H](C(=O)N[C@@H](CCC(N)=O)C(=O)NCc3ccc(S(C)(=O)=O)cc3)N2C(=O)[C@@H](NC(=O)c2cc3cc(C(=O)P(=O)(O)O)ccc3[nH]2)C1]

In some embodiments, STAT is

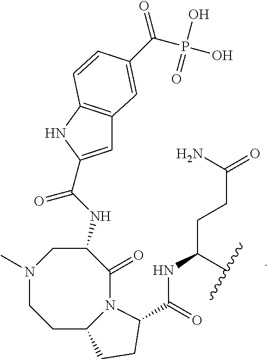 [SMILES: *[C@H](CCC(N)=O)NC(=O)[C@@H]1CC[C@@H]2CCN(C)C[C@H](NC(=O)c3cc4cc(C(=O)P(=O)(O)O)ccc4[nH]3)C(=O)N21]

In some embodiments, STAT is

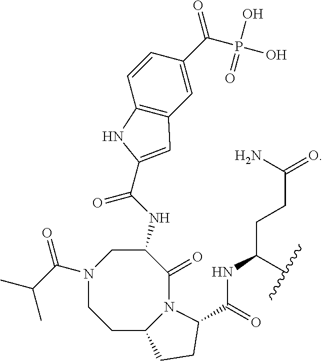 [SMILES: *[C@H](CCC(N)=O)NC(=O)[C@@H]1CC[C@@H]2CCN(C(=O)C(C)C)C[C@H](NC(=O)c3cc4cc(C(=O)P(=O)(O)O)ccc4[nH]3)C(=O)N21]

In some emodimets STAT is

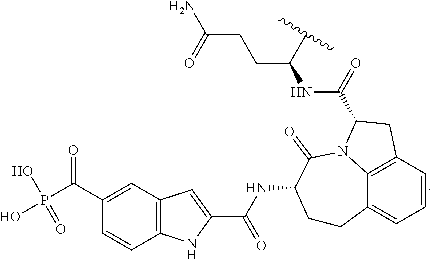 [SMILES: *[C@H](CCC(N)=O)NC(=O)[C@@H]1Cc2cccc3c2N1C(=O)[C@@H](NC(=O)c1cc2cc(C(=O)P(=O)(O)O)ccc2[nH]1)CC3]

In some embodiments, STAT is

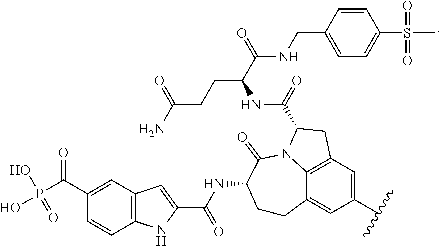 [SMILES: *c1cc2c3c(c1)C[C@@H](C(=O)N[C@@H](CCC(N)=O)C(=O)NCc1ccc(S(C)(=O)=O)cc1)N3C(=O)[C@@H](NC(=O)c1cc3cc(C(=O)P(=O)(O)O)ccc3[nH]1)CC2]

In some embodiments, STAT is

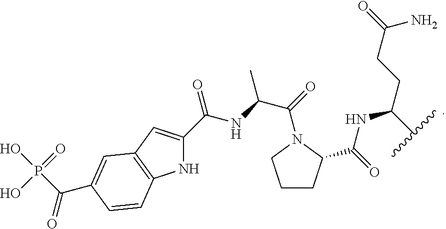 [SMILES: *[C@H](CCC(N)=O)NC(=O)[C@@H]1CCCN1C(=O)[C@H](C)NC(=O)c1cc2cc(C(=O)P(=O)(O)O)ccc2[nH]1]

In some embodiments, STAT is

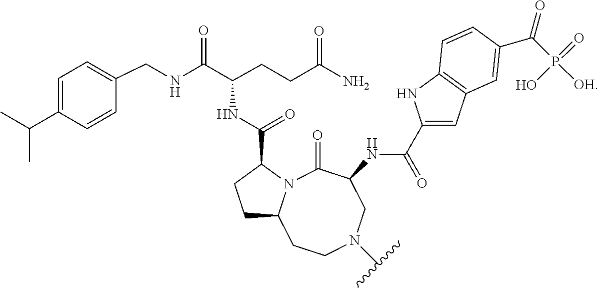 [SMILES: *N1CC[C@H]2CC[C@@H](C(=O)N[C@@H](CCC(N)=O)C(=O)NCc3ccc(C(C)C)cc3)N2C(=O)[C@@H](NC(=O)c2cc3cc(C(=O)P(=O)(O)O)ccc3[nH]2)C1]

In some embodiments, STAT is

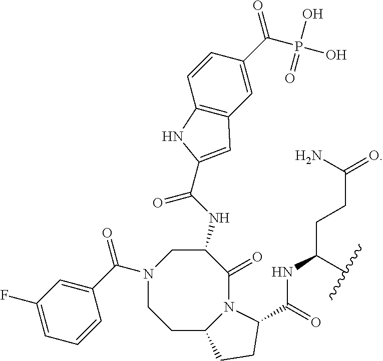 [SMILES: *[C@H](CCC(N)=O)NC(=O)[C@@H]1CC[C@@H]2CCN(C(=O)c3cccc(F)c3)C[C@H](NC(=O)c3cc4cc(C(=O)P(=O)(O)O)ccc4[nH]3)C(=O)N21]

In some embodiments, STAT is

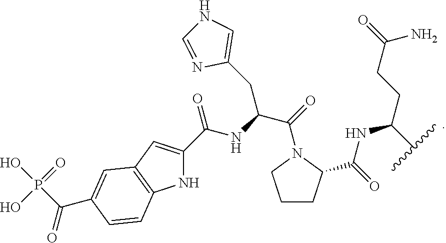 [SMILES: *[C@H](CCC(N)=O)NC(=O)[C@@H]1CCCN1C(=O)[C@H](Cc1c[nH]cn1)NC(=O)c1cc2cc(C(=O)P(=O)(O)O)ccc2[nH]1]

In some embodiments, STAT is

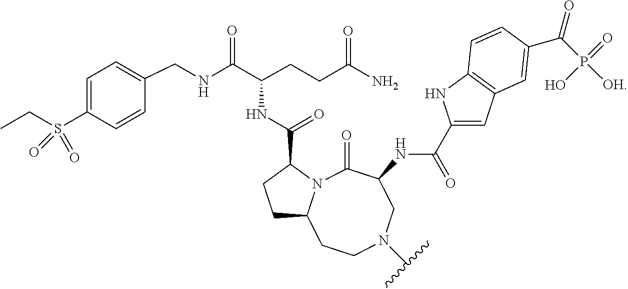 [SMILES: *N1CC[C@H]2CC[C@@H](C(=O)N[C@@H](CCC(N)=O)C(=O)NCc3ccc(S(=O)(=O)CC)cc3)N2C(=O)[C@@H](NC(=O)c2cc3cc(C(=O)P(=O)(O)O)ccc3[nH]2)C1]

In some embodiments, STAT is

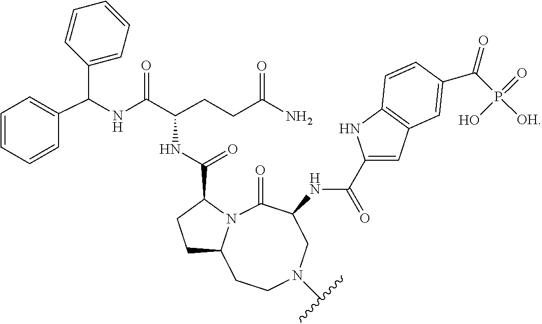 [SMILES: *N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(=O)P(=O)(O)O)ccc3[nH]2)C1]

In some embodiments, STAT is

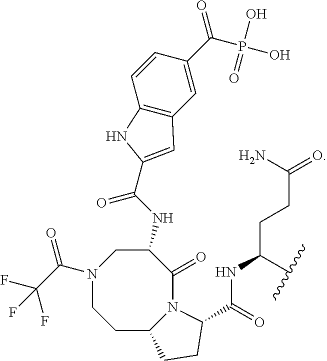 [SMILES: *[C@H](CCC(N)=O)NC(=O)[C@@H]1CC[C@@H]2CCN(C(=O)C(F)(F)F)C[C@H](NC(=O)c3cc4cc(C(=O)P(=O)(O)O)ccc4[nH]3)C(=O)N21]

In some embodiments, STAT is

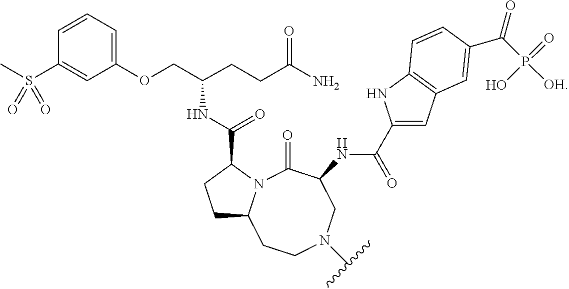 [SMILES: *N1CC[C@H]2CC[C@@H](C(=O)N[C@@H](CCC(N)=O)COc3cccc(S(C)(=O)=O)c3)N2C(=O)[C@@H](NC(=O)c2cc3cc(C(=O)P(=O)(O)O)ccc3[nH]2)C1]

In some embodiments, STAT is

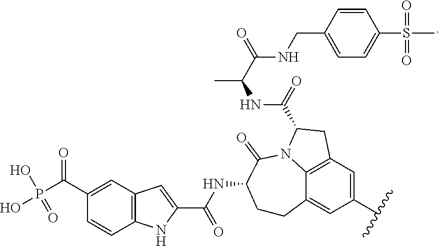 [SMILES: *c1cc2c3c(c1)C[C@@H](C(=O)N[C@@H](C)C(=O)NCc1ccc(S(C)(=O)=O)cc1)N3C(=O)[C@@H](NC(=O)c1cc3cc(C(=O)P(=O)(O)O)ccc3[nH]1)CC2]

In some embodiments, STAT is

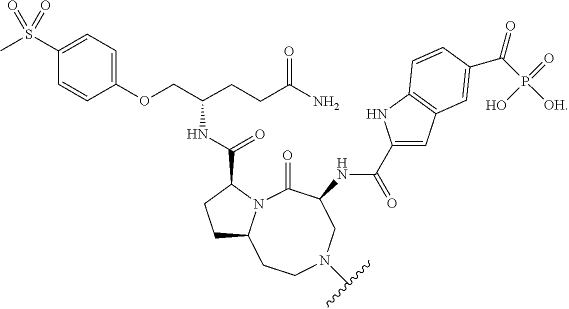 [SMILES: *N1CC[C@H]2CC[C@@H](C(=O)N[C@@H](CCC(N)=O)COc3ccc(S(C)(=O)=O)cc3)N2C(=O)[C@@H](NC(=O)c2cc3cc(C(=O)P(=O)(O)O)ccc3[nH]2)C1]

In some embodiments, STAT is

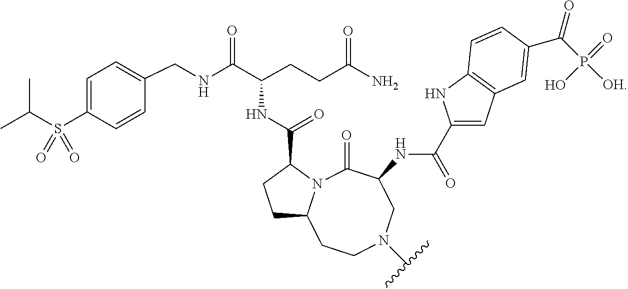 [SMILES: *N1CC[C@H]2CC[C@@H](C(=O)N[C@@H](CCC(N)=O)C(=O)NCc3ccc(S(=O)(=O)C(C)C)cc3)N2C(=O)[C@@H](NC(=O)c2cc3cc(C(=O)P(=O)(O)O)ccc3[nH]2)C1]

In some embodiments, STAT is

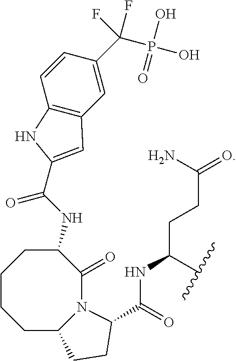 [SMILES: *[C@H](CCC(N)=O)NC(=O)[C@@H]1CC[C@@H]2CCCC[C@H](NC(=O)c3cc4cc(C(F)(F)P(=O)(O)O)ccc4[nH]3)C(=O)N21]

In some embodiments, STAT is

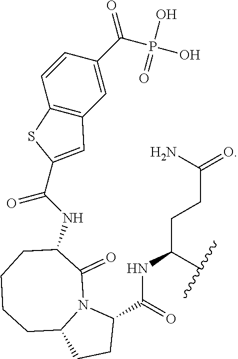 [SMILES: *[C@H](CCC(N)=O)NC(=O)[C@@H]1CC[C@@H]2CCCC[C@H](NC(=O)c3cc4cc(C(=O)P(=O)(O)O)ccc4s3)C(=O)N21]

In some embodiments, STAT is

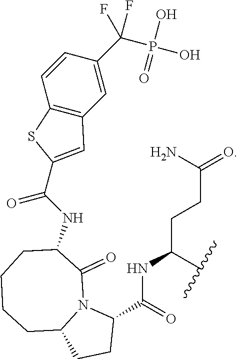 [SMILES: *[C@H](CCC(N)=O)NC(=O)[C@@H]1CC[C@@H]2CCCC[C@H](NC(=O)c3cc4cc(C(F)(F)P(=O)(O)O)ccc4s3)C(=O)N21]

In some embodiments, STAT is

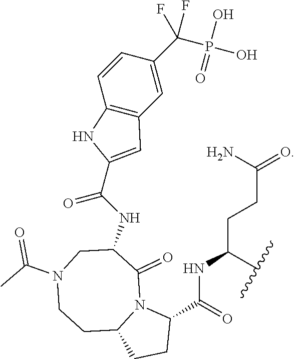 [SMILES: *[C@H](CCC(N)=O)NC(=O)[C@@H]1CC[C@@H]2CCN(C(C)=O)C[C@H](NC(=O)c3cc4cc(C(F)(F)P(=O)(O)O)ccc4[nH]3)C(=O)N21]

In some embodiments, STAT is

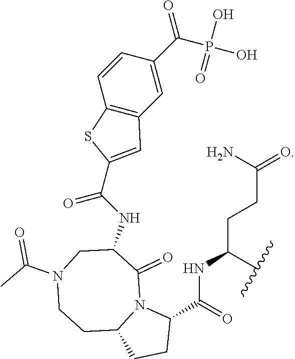 [SMILES: *[C@H](CCC(N)=O)NC(=O)[C@@H]1CC[C@@H]2CCN(C(C)=O)C[C@H](NC(=O)c3cc4cc(C(=O)P(=O)(O)O)ccc4s3)C(=O)N21]

In some embodiments, STAT is

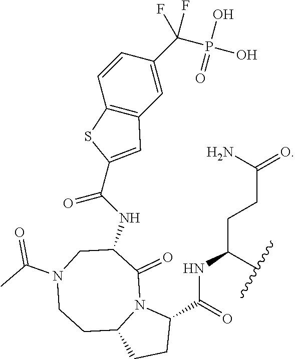 [SMILES: *[C@H](CCC(N)=O)NC(=O)[C@@H]1CC[C@@H]2CCN(C(C)=O)C[C@H](NC(=O)c3cc4cc(C(F)(F)P(=O)(O)O)ccc4s3)C(=O)N21]

In some embodiments, STAT is

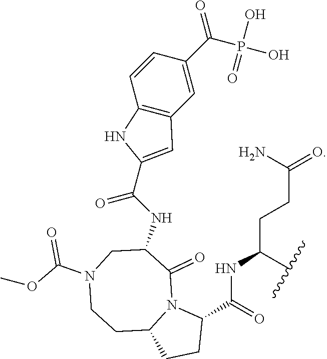 [SMILES: *[C@H](CCC(N)=O)NC(=O)[C@@H]1CC[C@@H]2CCN(C(=O)OC)C[C@H](NC(=O)c3cc4cc(C(=O)P(=O)(O)O)ccc4[nH]3)C(=O)N21]

In some embodiments, STAT is

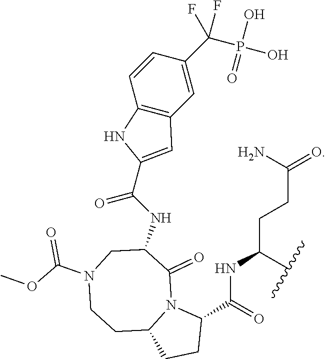 [SMILES: *[C@H](CCC(N)=O)NC(=O)[C@@H]1CC[C@@H]2CCN(C(=O)OC)C[C@H](NC(=O)c3cc4cc(C(F)(F)P(=O)(O)O)ccc4[nH]3)C(=O)N21]

In some embodiments, STAT is

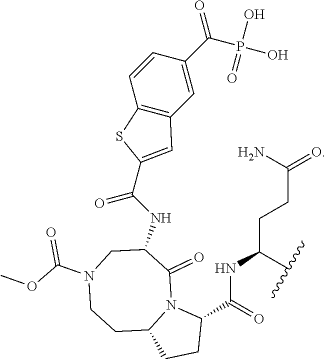 [SMILES: *[C@H](CCC(N)=O)NC(=O)[C@@H]1CC[C@@H]2CCN(C(=O)OC)C[C@H](NC(=O)c3cc4cc(C(=O)P(=O)(O)O)ccc4s3)C(=O)N21]

In some embodiments, STAT is

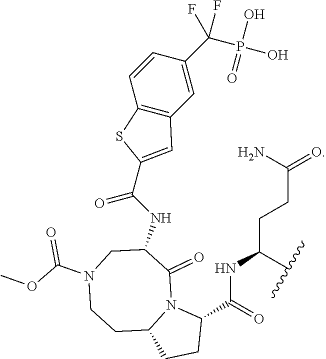 [SMILES: *[C@H](CCC(N)=O)NC(=O)[C@@H]1CC[C@@H]2CCN(C(=O)OC)C[C@H](NC(=O)c3cc4cc(C(F)(F)P(=O)(O)O)ccc4s3)C(=O)N21]

### In

some embodiments, STAT is

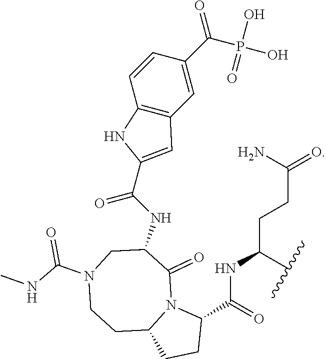 [SMILES: *[C@H](CCC(N)=O)NC(=O)[C@@H]1CC[C@@H]2CCN(C(=O)NC)C[C@H](NC(=O)c3cc4cc(C(=O)P(=O)(O)O)ccc4[nH]3)C(=O)N21]

In some embodiments, STAT is

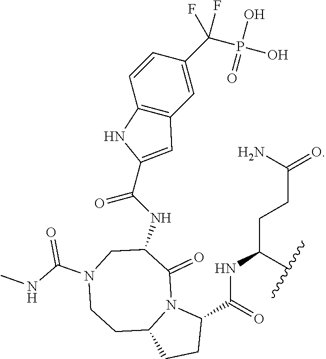 [SMILES: *[C@H](CCC(N)=O)NC(=O)[C@@H]1CC[C@@H]2CCN(C(=O)NC)C[C@H](NC(=O)c3cc4cc(C(F)(F)P(=O)(O)O)ccc4[nH]3)C(=O)N21]

In some embodiments, STAT is

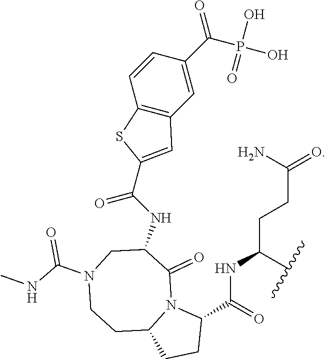 [SMILES: *[C@H](CCC(N)=O)NC(=O)[C@@H]1CC[C@@H]2CCN(C(=O)NC)C[C@H](NC(=O)c3cc4cc(C(=O)P(=O)(O)O)ccc4s3)C(=O)N21]

In some embodiments, STAT is

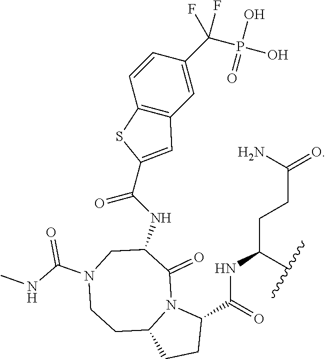 [SMILES: *[C@H](CCC(N)=O)NC(=O)[C@@H]1CC[C@@H]2CCN(C(=O)NC)C[C@H](NC(=O)c3cc4cc(C(F)(F)P(=O)(O)O)ccc4s3)C(=O)N21]

In some embodiments, the present invention provides a compound of formula I-a, wherein X¹, X², X³, R¹, and Ring A is

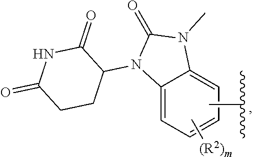

n is 1, and Q is —C(O)— as shown, to provide a compound of formula I-a-1:

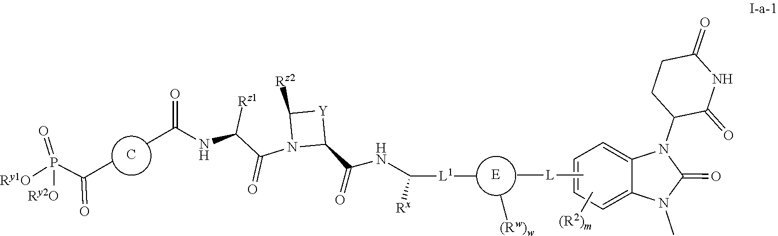

or a pharmaceutically acceptable salt thereof, wherein each of R², m, L, L¹, Ring C, Ring E, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-a, wherein X¹, X², X³, R¹, and Ring A is

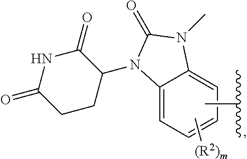

### R^{x} is

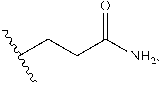 [SMILES: *CCC(N)=O]

n is 1, and Q is —C(O)— as shown, to provide a compound of formula I-a-2:

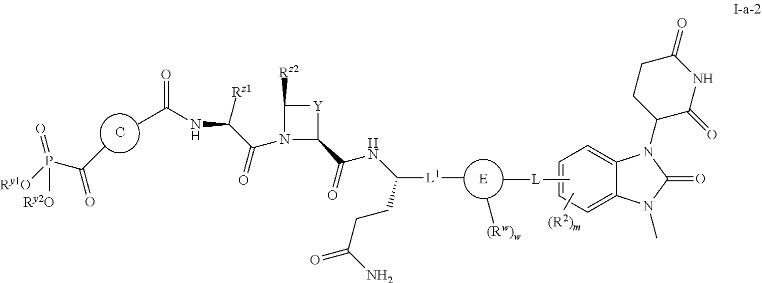

or a pharmaceutically acceptable salt thereof, wherein each of R², m, L, L¹, Ring C, Ring E, Y, R^{w}, w, R^{y1}, R^{y2}, R^{z1}, and R^{z2} is as defined above and described in embodiments herein, both singly and in combination.

In some embodiments, the present invention provides a compound of formula I-a, wherein X¹, X², X³, R¹, and Ring A is

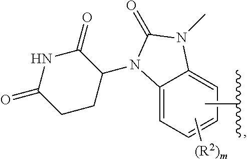

### Y is

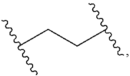 [SMILES: *CC*]

R^{z2} is hydrogen, n is 1, and Q is —C(O)— as shown, to provide a compound of formula I-a-3:

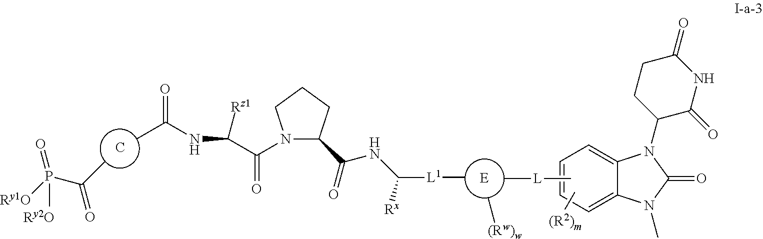

or a pharmaceutically acceptable salt thereof, wherein each of R², m, L, L¹, Ring C, Ring E, R^{w}, w, R^{x}, R^{y1}, R^{y2}, and R^{z1} is as defined above and described in embodiments herein, both singly and in combination.

In some embodiments, the present invention provides a compound of formula I-a, wherein X¹, X², X³, R¹, and Ring A is

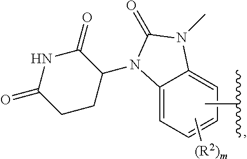

R^{z1} and R^{z2} are cyclically linked by

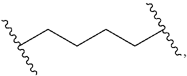 [SMILES: *CCCC*]

### Y is

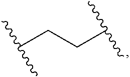 [SMILES: *CC*]

n is 1, and Q is —C(O)— as shown, to provide a compound of formula I-a-4:

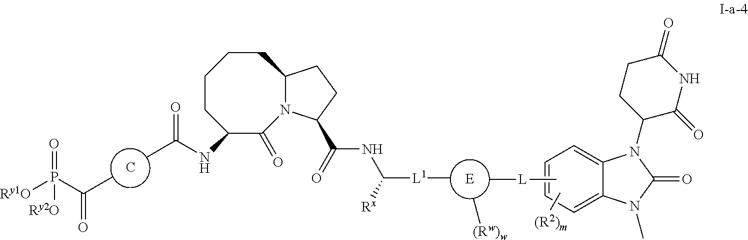

or a pharmaceutically acceptable salt thereof, wherein each of R², m, L, L¹, Ring C, Ring E, R^{w}, w, R^{x}, R^{y1}, and R^{y2} is as defined above and described in embodiments herein, both singly and in combination.

In some embodiments, the present invention provides a compound of formula I-a, wherein X¹, X², X³, R¹, and Ring A is

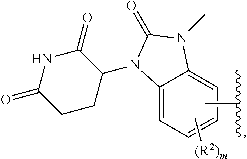

### Ring C is

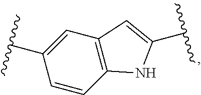 [SMILES: *c1ccc2[nH]c(*)cc2c1]

n is 1, and Q is —C(O)— as shown, to provide a compound of formula I-a-5:

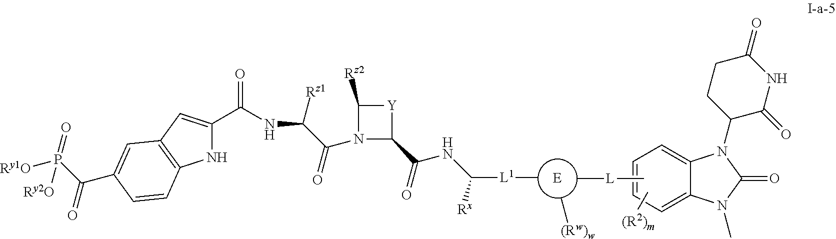

or a pharmaceutically acceptable salt thereof, wherein each of R² m, L, L¹, Ring E, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-a, wherein X¹, X², X³, R, and Ring A is

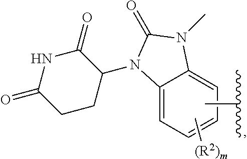

### n is 1, L¹ is

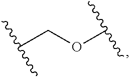 [SMILES: *CO*]

Ring E is phenylenyl, and Q is —C(O)— as shown, to provide a compound of formula I-a-6:

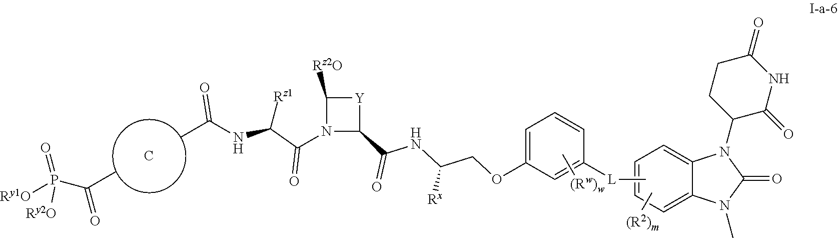

or a pharmaceutically acceptable salt thereof, wherein each of R², m, L, Ring C, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-a, wherein X¹, X², X³, R¹, and Ring A is

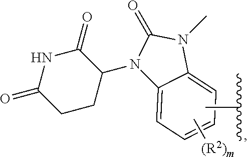

R^{z1} and R^{z2} are cyclically linked by

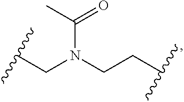 [SMILES: *CCN(C*)C(C)=O]

### Y is

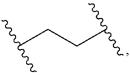 [SMILES: *CC*]

n is 1, and Q is —C(O)— as shown, to provide a compound of formula I-a-7:

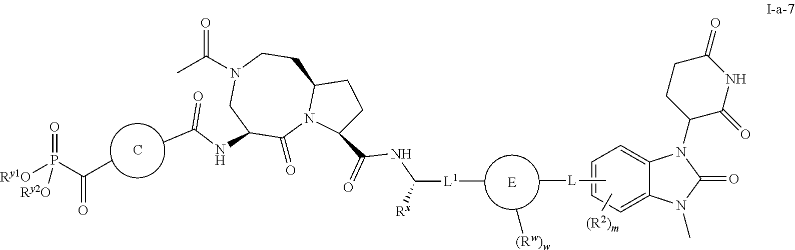

or a pharmaceutically acceptable salt thereof, wherein each of R², m, L, L¹, Ring C, Ring E, R^{w}, w, R^{x}, R^{y1}, and R^{y2} is as defined above and described in embodiments herein, both singly and in combination.

In some embodiments, the resent invention provides a compound of formula I-b, wherein Ring D is phenyl, p is 1, R⁷ is

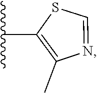 [SMILES: *c1scnc1C]

n is 1, and Q is —C(O)— as shown, to provide a compound of formula I-b-1:

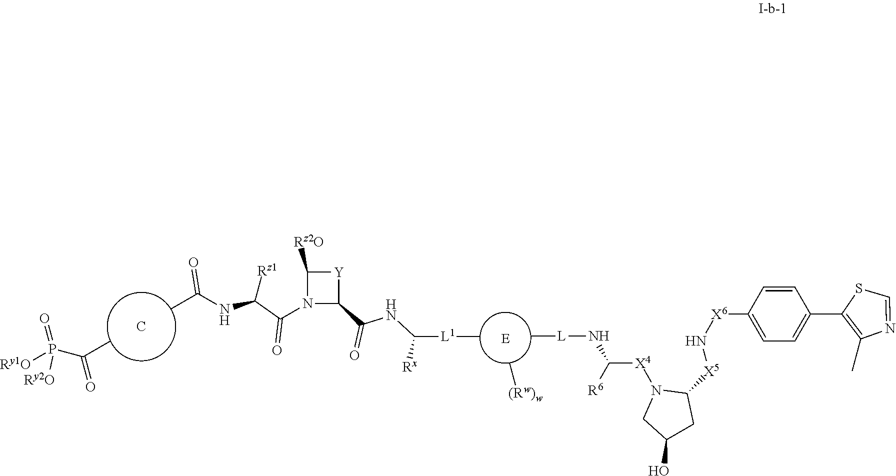

or a pharmaceutically acceptable salt thereof, wherein each of X⁴, X⁵, X⁶, R⁶, L, L¹, Ring C, Ring E, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-b, wherein Ring D is phenyl, p is 1, R⁷ is

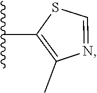 [SMILES: *c1scnc1C]

### R^{x} is

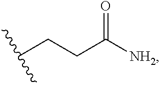 [SMILES: *CCC(N)=O]

n is 1, and X⁴, X⁵, and Q is —C(O)— as shown, to provide a compound of formula I-b-2:

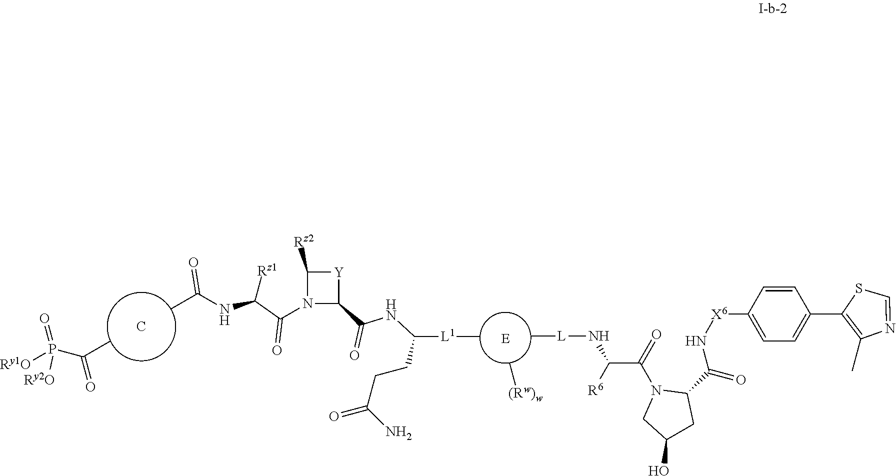

or a pharmaceutically acceptable salt thereof, wherein each of X⁶, R⁶, L, L¹, Ring C, Ring E, Y, R^{w}, w, R^{y1}, R^{y2}, R^{z1}, and R^{z2} is as defined above and described in embodiments herein, both singly and in combination.

In some embodiments, the present invention provides a compound of formula I-b, wherein Ring D is phenyl, p is 1, R⁷ is

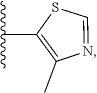 [SMILES: *c1scnc1C]

### Y is

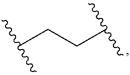 [SMILES: *CC*]

n is 1, and X⁴, X⁵, and Q is —C(O)— as shown, to provide a compound of formula I-b-3:

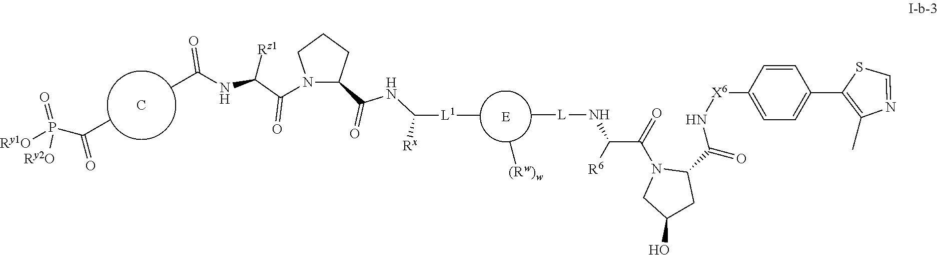

or a pharmaceutically acceptable salt thereof, wherein each of X⁶, R⁶, L, L¹, Ring C, Ring E, R^{w}, w, R^{x}, R^{y1}, R^{y2}, and R^{z1} is as defined above and described in embodiments herein, both singly and in combination.

In some embodiments, the present invention provides a compound of formula I-b, wherein Ring D is phenyl, p is 1, R⁷ is

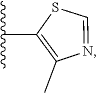 [SMILES: *c1scnc1C]

R^{z1} and R^{z2} are cyclically linked by

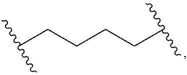 [SMILES: *CCCC*]

### Y is

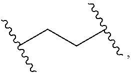 [SMILES: *CC*]

n is 1, and X⁴, X⁵, and Q is —C(O)— as shown, to provide a compound of formula I-b-4:

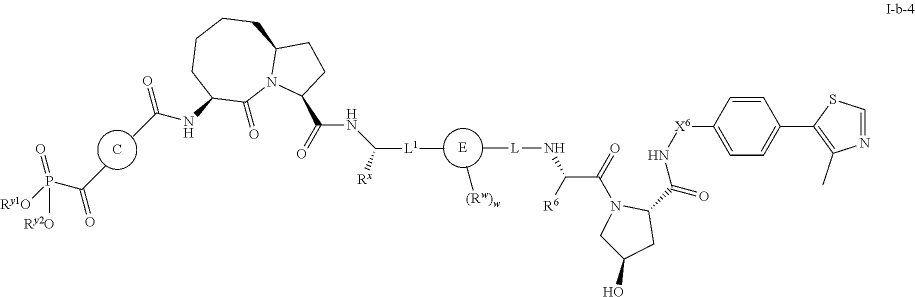

or a pharmaceutically acceptable salt thereof, wherein each of X⁶, R⁶, L, L¹, Ring C, Ring E, R^{w}, R^{x}, R^{y1}, and R^{y2} is as defined above and described in embodiments herein, both singly and in combination.

In some embodiments, the present invention provides a compound of formula I-b, wherein Ring D is phenyl, p is 1, R⁷ is

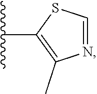 [SMILES: *c1scnc1C]

### Ring C is

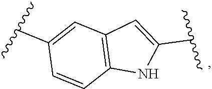 [SMILES: *c1ccc2[nH]c(*)cc2c1]

n is 1, and X⁴, X⁵, and Q is —C(O)— as shown, to provide a compound of formula I-b-5:

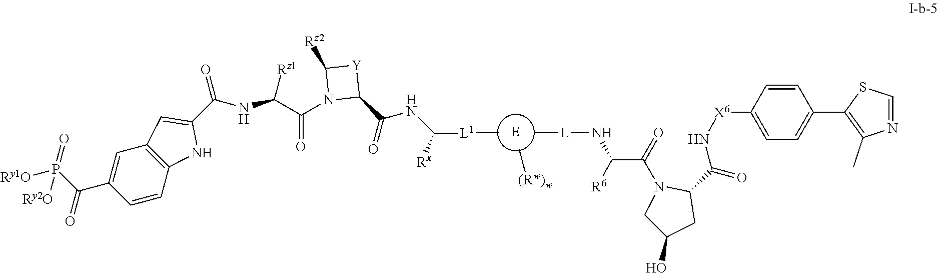

or a pharmaceutically acceptable salt thereof, wherein each of X⁶, R⁶, L, L¹, Ring E, Y, R^{w}, w, R^{x}, R^{●}, R^{y2}, R^{z1}, and R^{z2} is as defined above and described in embodiments herein, both singly and in combination.

In some embodiments, the present invention provides a compound of formula I-b, wherein Ring D is phenyl, p is 1, R⁷ is

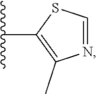 [SMILES: *c1scnc1C]

### n is 1, L¹ is

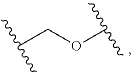 [SMILES: *CO*]

Ring E is phenylenyl, and X⁴, X⁵, and Q is —C(O)— as shown, to provide a compound of formula I-b-6:

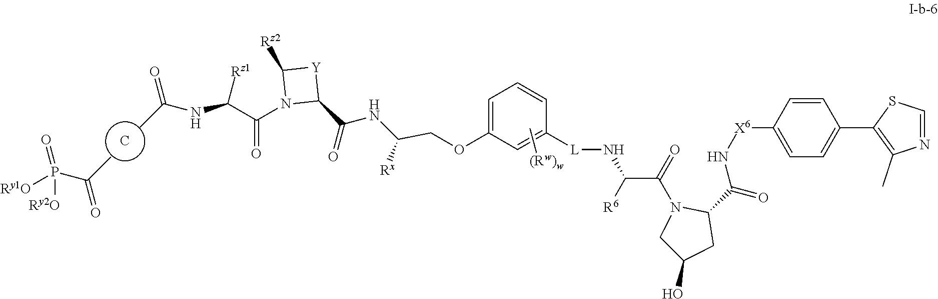

or a pharmaceutically acceptable salt thereof, wherein each of X⁶, R⁶, L, Ring C, X, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-b, wherein Ring D is phenyl, p is 1, R⁷ is

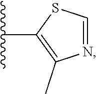 [SMILES: *c1scnc1C]

R^{z1} and R^{z2} are cyclically linked by

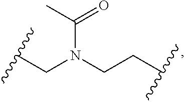 [SMILES: *CCN(C*)C(C)=O]

### Y is

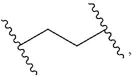 [SMILES: *CC*]

n is 1, and X⁴, X⁵, and Q is —C(O)— as shown, to provide a compound of formula I-b-7:

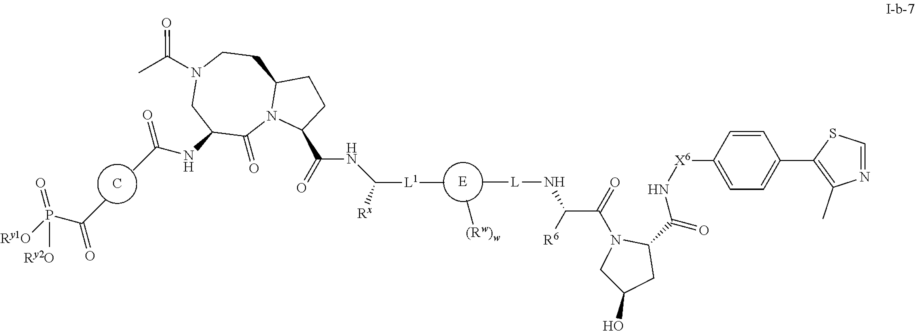

or a pharmaceutically acceptable salt thereof, wherein each of X⁶, R⁶, L, L¹, Ring C, Ring E, R^{w}, w, R^{x}, R^{y1}, and R^{y2} is as defined above and described in embodiments herein, both singly and in combination.

In some embodiments, the present invention provides a compound of formula I-b, wherein Ring D is phenyl, p is 1, R⁷ is

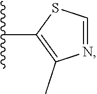 [SMILES: *c1scnc1C]

### n is 1, and L¹ is

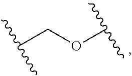 [SMILES: *CO*]

as shown, to provide a compound of formula I-b-8:

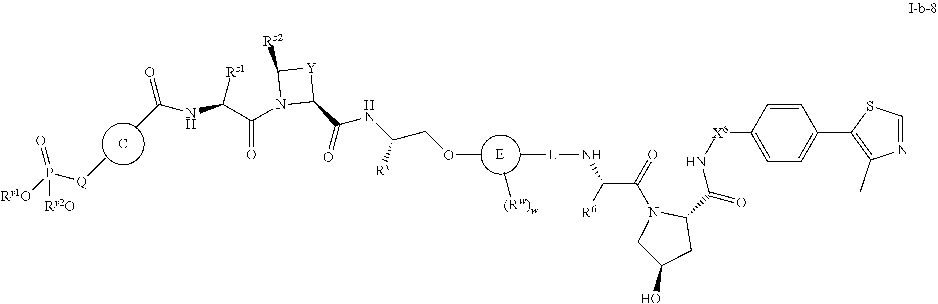

or a pharmaceutically acceptable salt thereof, wherein each of X⁴, X⁵, X⁶, R⁶, L, Ring C, Ring E, Q, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-b, wherein Ring D is phenyl, p is 1, R⁷ is

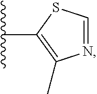 [SMILES: *c1scnc1C]

### n is 1, L¹ is

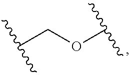 [SMILES: *CO*]

and X⁴, X⁵, and Q is —C(O)— as shown, to provide a compound of formula I-b-9:

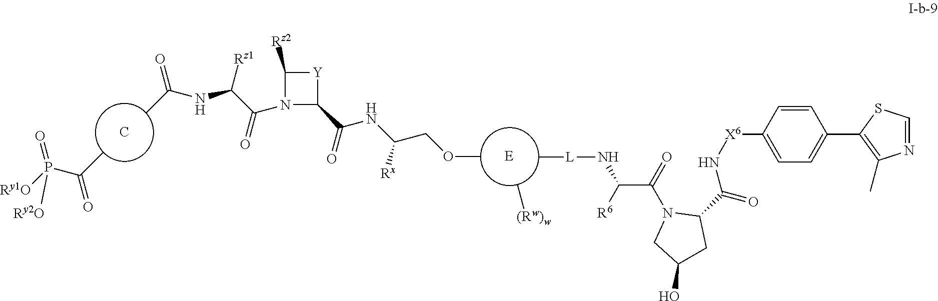

or a pharmaceutically acceptable salt thereof, wherein each of X⁶, R⁶, L, Ring C, Ring E, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-c, wherein X¹, X², X³, R¹, and Ring A is

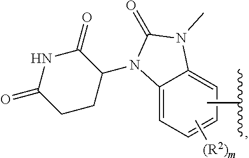

### X is

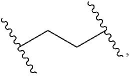 [SMILES: *CC*]

Y is —CH₂—, Ring F is a 6-member aryl, and Q is —C(O)— as shown, to provide a compound of formula I-c-1:

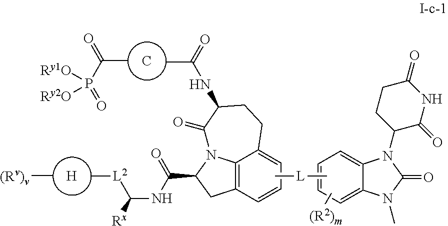

or a pharmaceutically acceptable salt thereof, wherein each of R², m, L, L², Ring C, Ring H, R^{x}, R^{y1}, R^{y2}, R^{v}, and v is as defined above and described in embodiments herein, both singly and in combination.

In some embodiments, the present invention provides a compound of formula I-c, wherein X¹, X², X³, R¹, and Ring A is

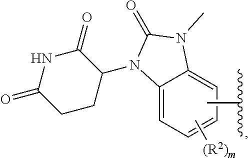

### Ring C is

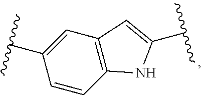 [SMILES: *c1ccc2[nH]c(*)cc2c1]

### X is

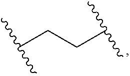 [SMILES: *CC*]

Y is —CH₂—, Ring F is a 6-member aryl, and Q is —C(O)— as shown, to provide a compound of formula I-c-2:

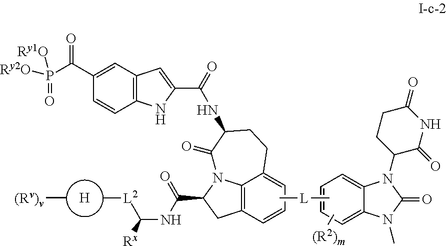

or a pharmaceutically acceptable salt thereof, wherein each of R², m, L, L², Ring H, R^{x}, RR, R^{z}, R, and v is as defined above and described in embodiments herein, both singly and in combination.

In some embodiments, the present invention provides a compound of formula I-c, wherein X¹, X², X³, R¹, and Ring A is

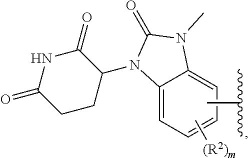

### L² is

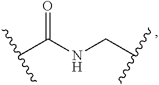 [SMILES: *CNC(*)=O]

Ring H is phenyl, X is

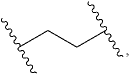 [SMILES: *CC*]

Y is —CH₂—, Ring F is a 6-member aryl, and Q is —C(O)— as shown, to provide a compound of formula I-c-3:

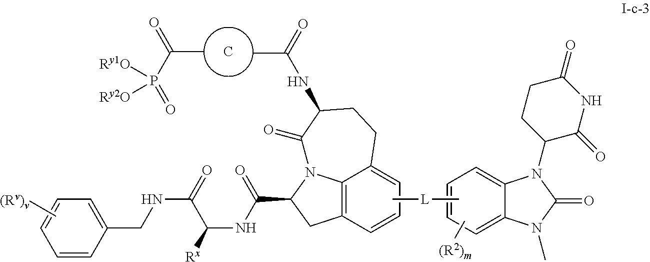

or a pharmaceutically acceptable salt thereof, wherein each of R², m, L, Ring C, R, R^{y1}, R^{y2}, R^{v}, and v is as defined above and described in embodiments herein, both singly and in combination.

In some embodiments, the present invention provides a compound of formula I-d, wherein X¹, X², X³, R¹, and Ring A is

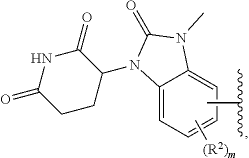

### n is 1, Y is

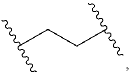 [SMILES: *CC*]

Ring G is an 8-membered heterocyclyl, and Q is —C(O)— as shown, to provide a compound of formula I-d-1:

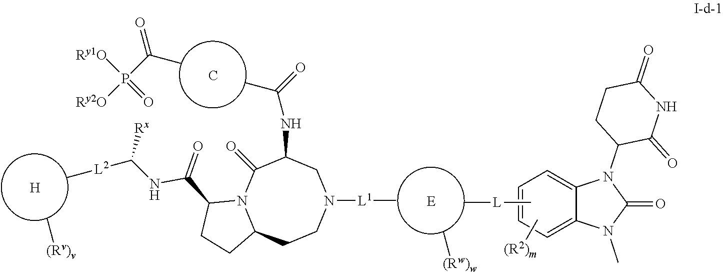

or a pharmaceutically acceptable salt thereof, wherein each of R², m, L, L¹, L², Ring C, Ring H, Ring E, R^{x}, R^{y1}, R^{y2}, R^{v}, v, R^{w}, and w is as defined above and described in embodiments herein, both singly and in combination.

In some embodiments, the present invention provides a compound of formula I-d, wherein X¹, X², X³, R¹, and Ring A is

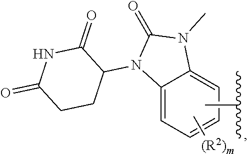

### n is 1, Y is

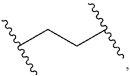 [SMILES: *CC*]

Ring G is an 8-membered heterocyclyl, Ring C is

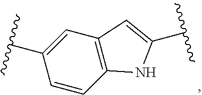 [SMILES: *c1ccc2[nH]c(*)cc2c1]

and Q is —C(O)— as shown, to provide a compound of formula I-d-2:

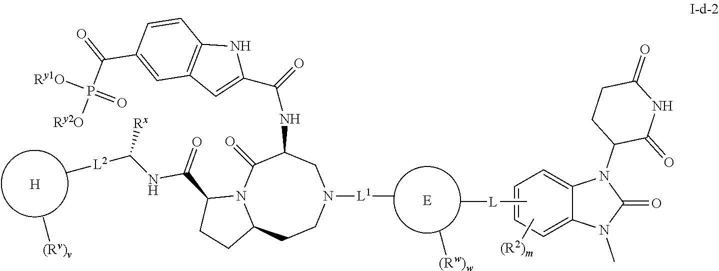

or a pharmaceutically acceptable salt thereof, wherein each of R², m, L, L¹, L², Ring H, Ring E, R^{x}, R^{y1}, R^{y2}, R, v, R^{w}, and w is as defined above and described in embodiments herein, both singly and in combination.

In some embodiments, the present invention provides a compound of formula I-d, wherein X¹, X², X³, R¹, and Ring A is

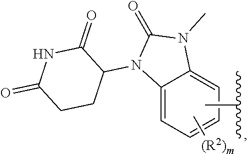

### n is 1, Y is

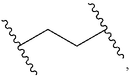 [SMILES: *CC*]

Ring G is an 8-membered heterocyclyl, L² is

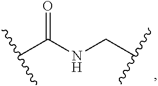 [SMILES: *CNC(*)=O]

Ring H is phenyl, and Q is —C(O)— as shown, to provide a compound of

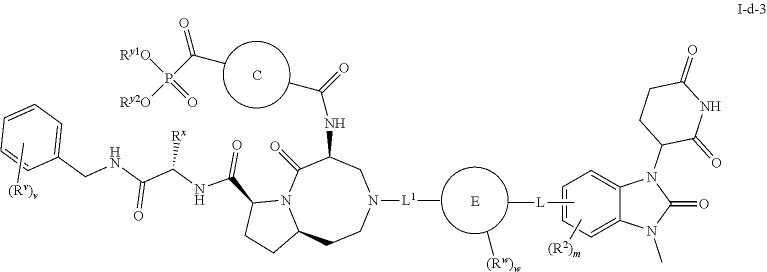

or a pharmaceutically acceptable salt thereof, wherein each of R², m, L, L¹, Ring C, Ring E, R^{x}, R^{y1}, R^{y2}, R^{v}, v, R^{w}, and w is as defined above and described in embodiments herein, both singly and in combination.

In some embodiments, the present invention provides a compound of formula I-d, wherein X¹, X², X³, R¹, and Ring A is

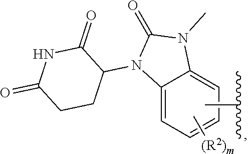

### n is 1, Y is

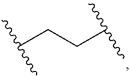 [SMILES: *CC*]

Ring G is an 8-membered heterocyclyl, Ring E is cyclohexyl, w is 0, and Q is —C(O)— as shown, to provide a compound of formula I-d-4:

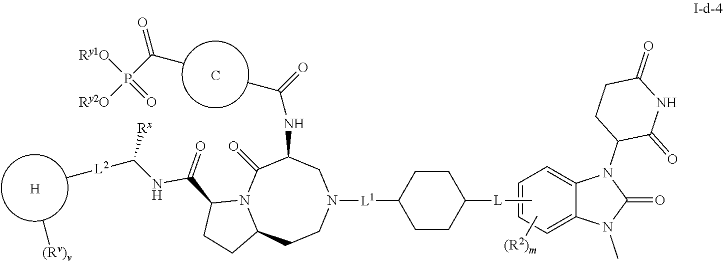

or a pharmaceutically acceptable salt thereof, wherein each of R², m, L, L², Ring C, Ring H, R^{x}, R^{y1}, R^{y2}, R^{v}, and v is as defined above and described in embodiments herein, both singly and in combination.

In some embodiments, the present invention provides a compound of formula I-e, wherein Ring D is phenyl, p is 1, R⁷ is

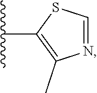 [SMILES: *c1scnc1C]

n is 1, and X⁴, X⁵, and Q are —C(O)— as shown, to provide a compound of formula I-e-1:

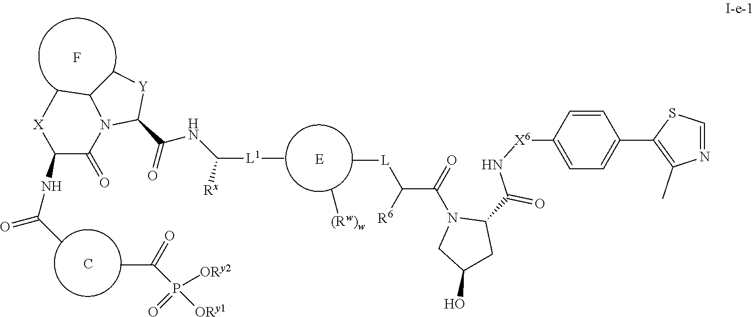

or a pharmaceutically acceptable salt thereof, wherein each of X⁶, R⁶, L, L¹, Ring C, Ring E, Ring F, X, Y, R^{w}, w, R^{x}, R^{y1}, and R^{y2} is as defined above and described in embodiments herein, both singly and in combination.

In some embodiments, the present invention provides a compound of formula I-e, wherein Ring D is phenyl, p is 1, R⁷ is

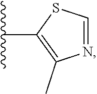 [SMILES: *c1scnc1C]

### n is 1, X is

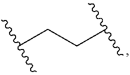 [SMILES: *CC*]

Y is —CH₂—, Ring F is a 6-member aryl, and X⁴, X⁵, and Q are —C(O)— as shown, to provide a compound of formula I-e-2:

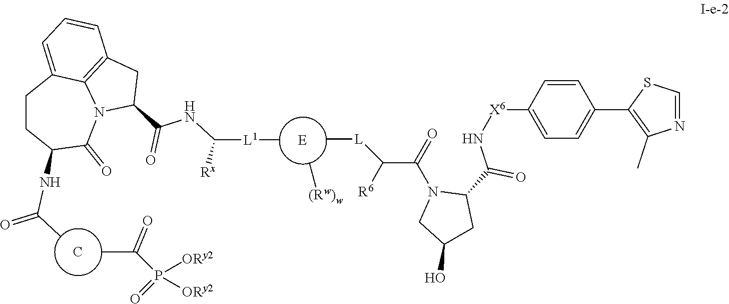

or a pharmaceutically acceptable salt thereof, wherein each of X⁶, R⁶, L, L, Ring C, Ring E, R^{w}, w, R^{x}, R^{y1}, and R^{y2} is as defined above and described in embodiments herein, both singly and in combination.

In some embodiments, the present invention provides a compound of formula I-f, wherein Ring D is phenyl, p is 1, R⁷ is

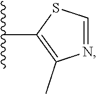 [SMILES: *c1scnc1C]

### n is 1, and L¹ is

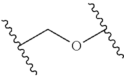 [SMILES: *CO*]

as shown, to provide a compound of formula I-f-1:

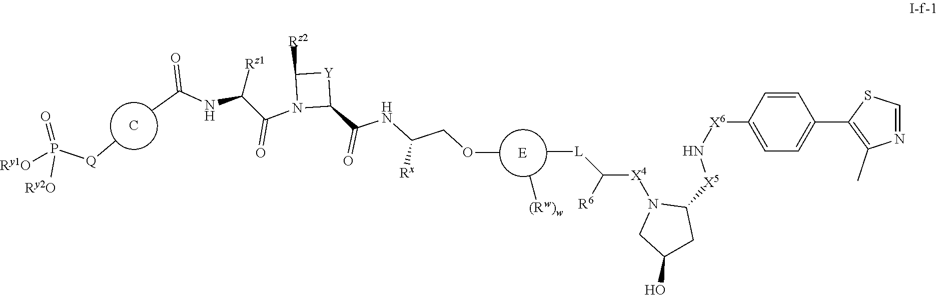

or a pharmaceutically acceptable salt thereof, wherein each of X⁴, X⁵, X⁶, R⁶, L, Ring C, Ring E, Q, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-f, wherein Ring D is phenyl, p is 1, R⁷ is

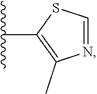 [SMILES: *c1scnc1C]

### n is 1, L¹ is

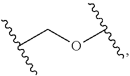 [SMILES: *CO*]

and X⁴, X⁵, and Q is —C(O)— as shown, to provide a compound of formula I-f-2:

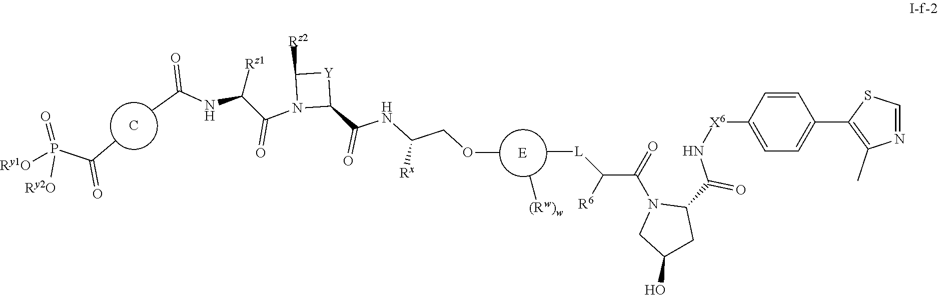

or a pharmaceutically acceptable salt thereof, wherein each of X⁶, R⁶, L, Ring C, Ring E, Y, R^{w}, w, R^{x}, R^{y1}, R^{y2}, R^{z1}, and R^{z2} is as defined above and described in embodiments herein, both singly and in combination.

In some embodiments, the present invention provides a compound of formula I-f, wherein Ring D is phenyl, p is 1, R⁷ is

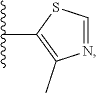 [SMILES: *c1scnc1C]

R^{z1} and R^{z2} are cyclically linked by

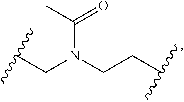 [SMILES: *CCN(C*)C(C)=O]

### Y is n is 1 L¹ is

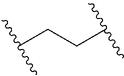 [SMILES: *CC*]

### n is 1, L¹ is

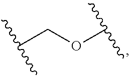 [SMILES: *CO*]

and X⁴, X⁵, and Q is —C(O)— as shown, to provide a compound of formula I-f-3:

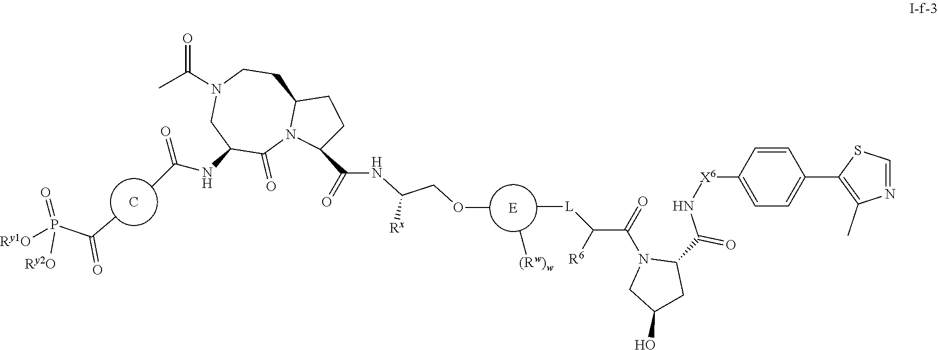

or a pharmaceutically acceptable salt thereof, wherein each of X⁶, R⁶, L, Ring C, Ring E, R^{w}, w, R^{x}, R^{y1}, and R^{y2} is as defined above and described in embodiments herein, both singly and in combination.

In some embodiments, the present invention provides a compound of formula I-g, wherein q is 0 and Ring M, L³, Ring N are

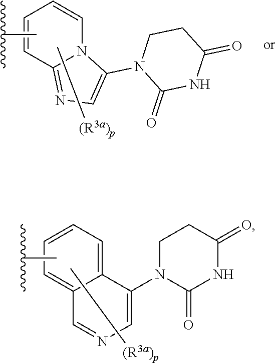

### and STAT is

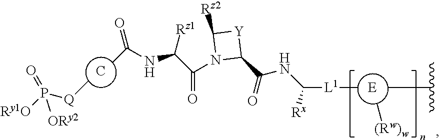

wherein n is 1, and Q is —C(O)— as shown, to provide a compound of formulae I-g-1 or I-g-2:

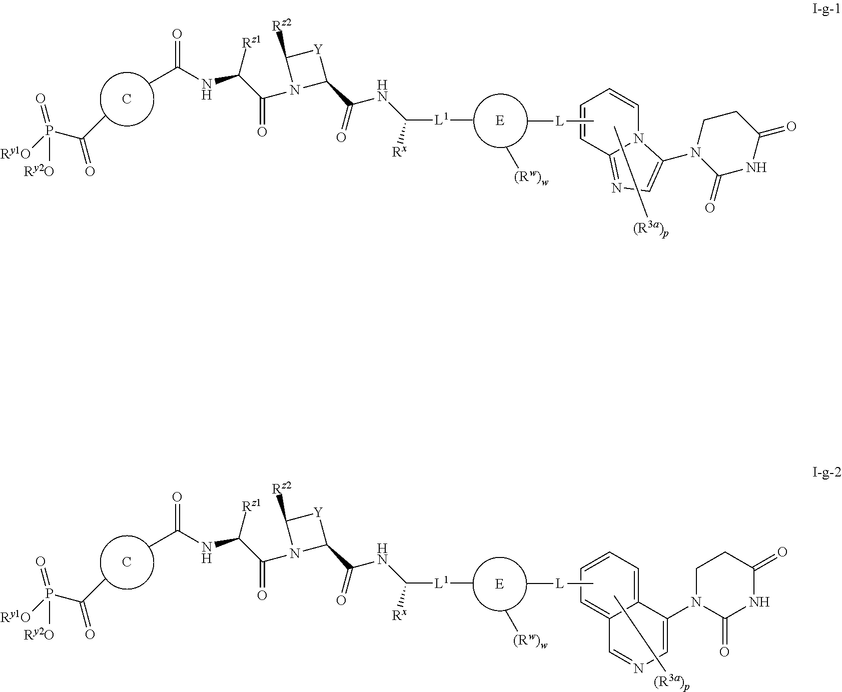

or a pharmaceutically acceptable salt thereof, wherein each of R^{3a}, p, L, L¹, Ring C, Ring E, Y, R^{w}, w, R^{x},
R^{y1}, R^{y2}, R^{z1}, and R^{z2} is as defined above and described in embodiments herein, both singly and in combination.

In some embodiments, the present invention provides a compound of formula I-g, wherein q is 0 and Ring M, L³, Ring N are

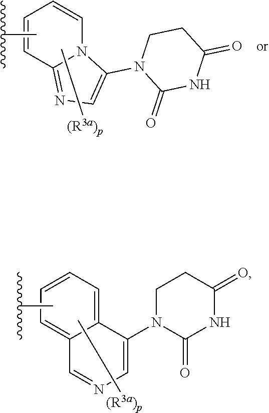

### and STAT is

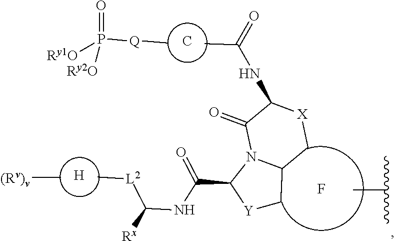

wherein X is

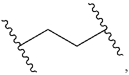 [SMILES: *CC*]

Y is —CH₂—, Ring F is a 6-member aryl, and Q is —C(O)— as shown, to provide a compound of formulae I-g-3 or I-g-4:

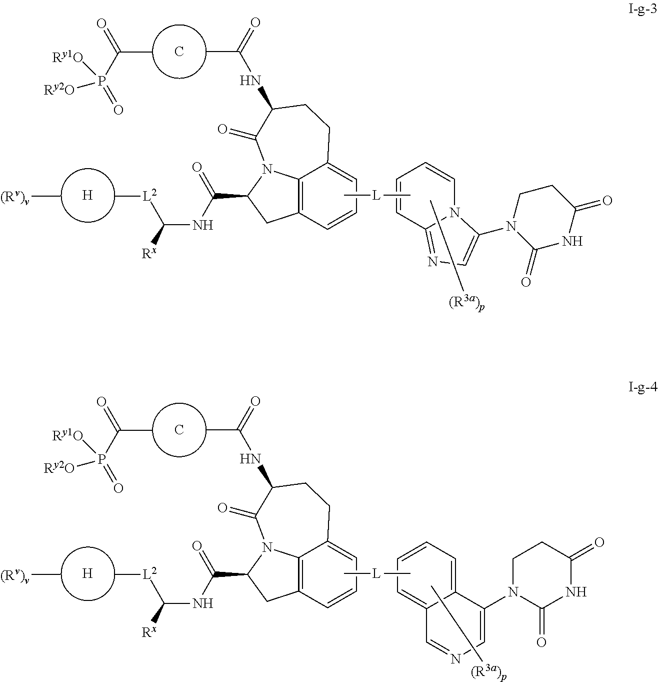

or a pharmaceutically acceptable salt thereof, wherein each of R^{3a}, p, L, L², Ring C, Ring H, R^{v}, v, R^{x}, R^{y1}, and R^{y2} is as defined above and described in embodiments herein, both singly and in combination.

In some embodiments, the present invention provides a compound of formula I-g, wherein q is 0 and Ring M, L³, Ring N are

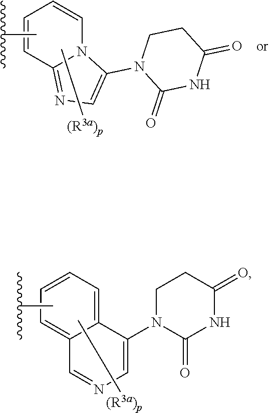

### and STAT is

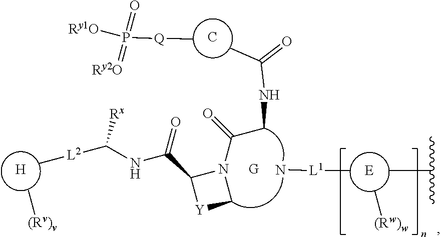

wherein is 1, Y is

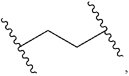 [SMILES: *CC*]

Ring G is an 8-membered heterocyclyl, and Q is —C(O)— as shown, to provide a compound of formulae I-g-5 or I-g-6:

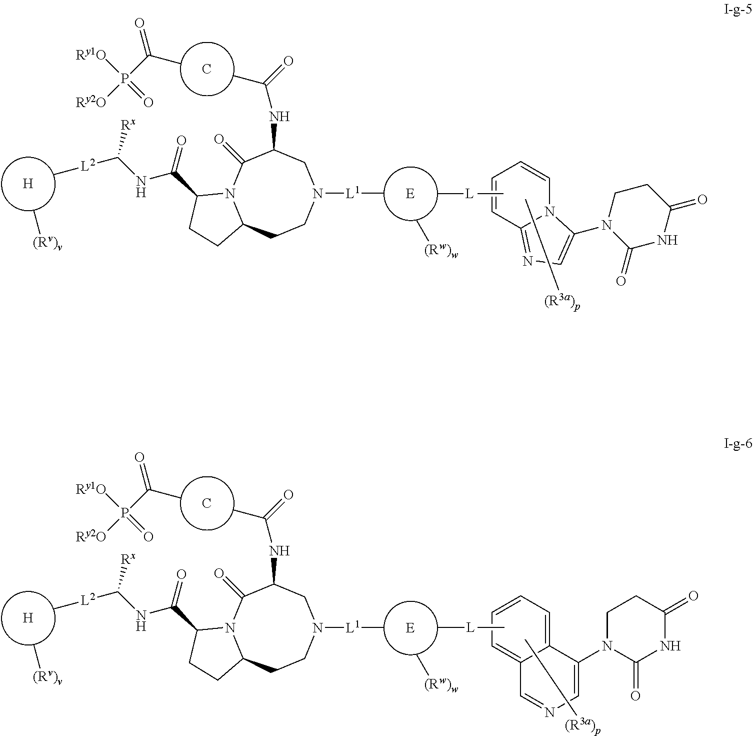

or a pharmaceutically acceptable salt thereof, wherein each of R^{3a}, p, L, L¹, L², Ring C, Ring H, Ring E, R^{v}, v, R^{w}, w, R^{x}, R^{y1}, and R^{y2} is as defined above and described in embodiments herein, both singly and in combination.

In some embodiments, the present invention provides a compound of formula I-g, wherein q is 0 and Ring M, L³, Ring N are

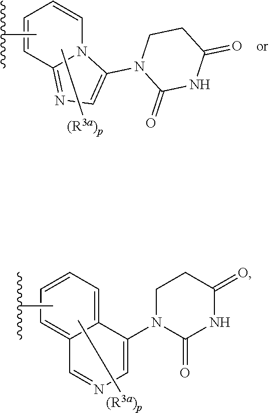

### and STAT is

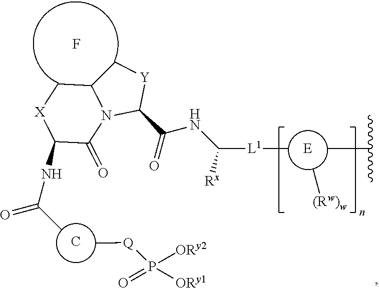

wherein n is 1, X is

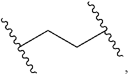 [SMILES: *CC*]

Y is —CH₂—, Ring F is a 6-member aryl, and Q is —C(O)— as shown, to provide a compound of formulae I-g-7 or I-g-8:

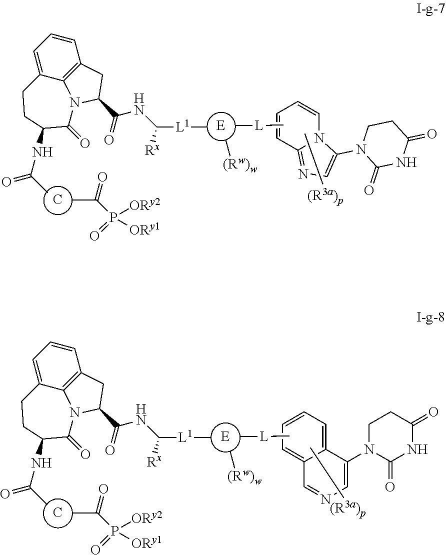

or a pharmaceutically acceptable salt thereof, wherein each of R^{3a}, p, L, L¹, Ring C, Ring E, R^{w}, w, R^{x}, R^{y1}, and R^{y2} is as defined above and described in embodiments herein, both singly and in combination.

Exemplary compounds of the invention are set forth in Table 1 and Table 1A, below.

TABLE 1
Exemplary Compounds

| I-# | Structure |
|---|---|
| I-1 | 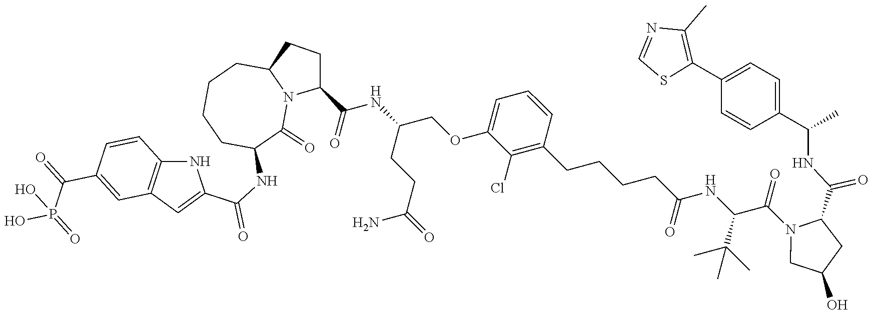 [SMILES: Cc1ncsc1-c1ccc([C@H](C)NC(=O)[C@@H]2C[C@@H](O)CN2C(=O)[C@@H](NC(=O)CCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)C(C)(C)C)cc1] |
| I-2 | 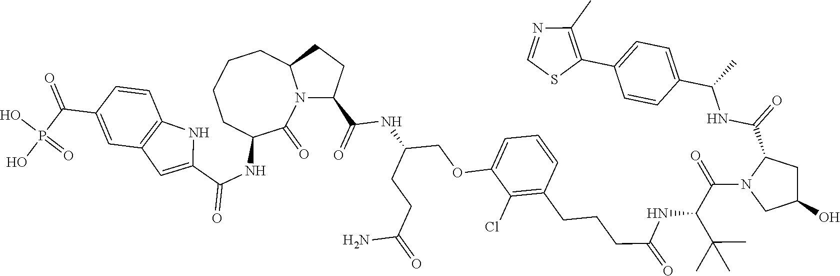 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)C(C)(C)C)cc1] |
| I-3 | 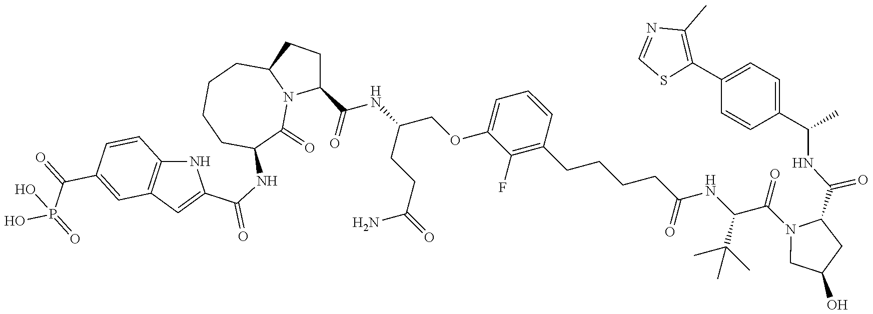 [SMILES: Cc1ncsc1-c1ccc([C@H](C)NC(=O)[C@@H]2C[C@@H](O)CN2C(=O)[C@@H](NC(=O)CCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2F)C(C)(C)C)cc1] |
| I-4 | 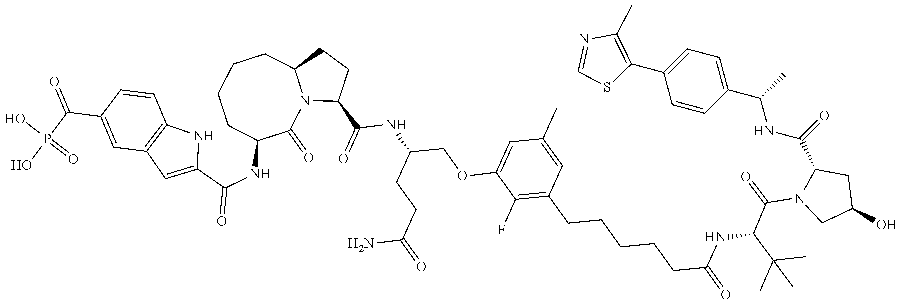 [SMILES: Cc1cc(CCCCCC(=O)N[C@H](C(=O)N2C[C@H](O)C[C@H]2C(=O)N[C@@H](C)c2ccc(-c3scnc3C)cc2)C(C)(C)C)c(F)c(OC[C@H](CCC(N)=O)NC(=O)[C@@H]2CC[C@@H]3CCCC[C@H](NC(=O)c4cc5cc(C(=O)P(=O)(O)O)ccc5[nH]4)C(=O)N32)c1] |
| I-5 | 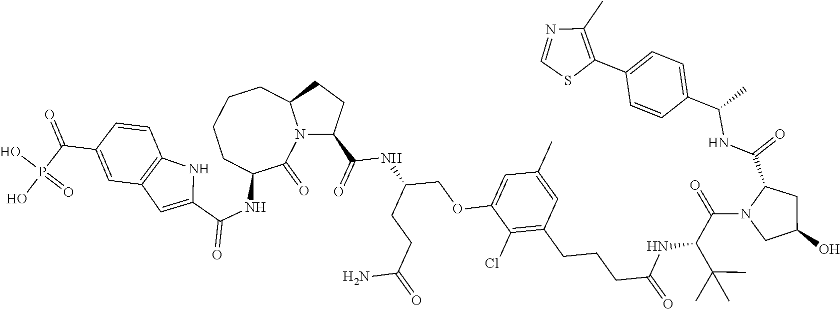 [SMILES: Cc1cc(CCCC(=O)N[C@H](C(=O)N2C[C@H](O)C[C@H]2C(=O)N[C@@H](C)c2ccc(-c3scnc3C)cc2)C(C)(C)C)c(Cl)c(OC[C@H](CCC(N)=O)NC(=O)[C@@H]2CC[C@@H]3CCCC[C@H](NC(=O)c4cc5cc(C(=O)P(=O)(O)O)ccc5[nH]4)C(=O)N32)c1] |
| I-6 | 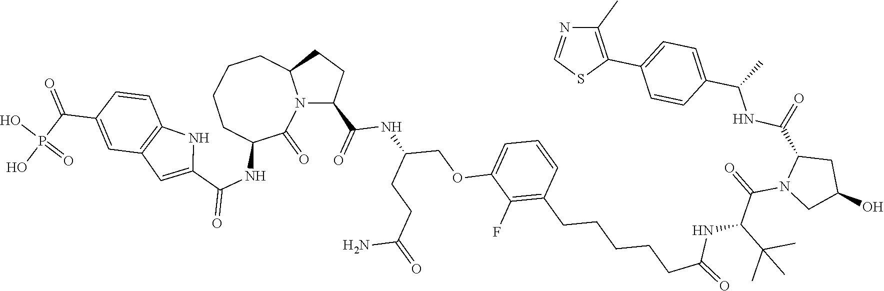 [SMILES: Cc1ncsc1-c1ccc([C@H](C)NC(=O)[C@@H]2C[C@@H](O)CN2C(=O)[C@@H](NC(=O)CCCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2F)C(C)(C)C)cc1] |
| I-7 | 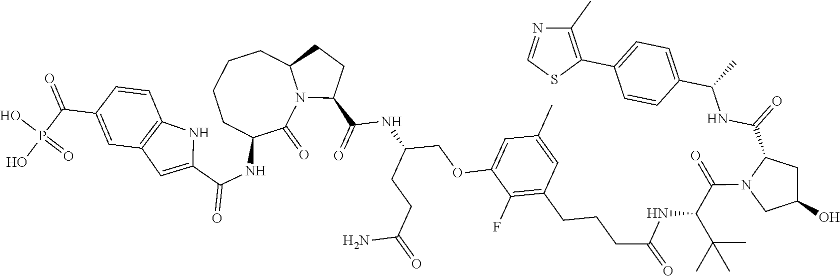 [SMILES: Cc1cc(CCCC(=O)N[C@H](C(=O)N2C[C@H](O)C[C@H]2C(=O)N[C@@H](C)c2ccc(-c3scnc3C)cc2)C(C)(C)C)c(F)c(OC[C@H](CCC(N)=O)NC(=O)[C@@H]2CC[C@@H]3CCCC[C@H](NC(=O)c4cc5cc(C(=O)P(=O)(O)O)ccc5[nH]4)C(=O)N32)c1] |
| I-8 | 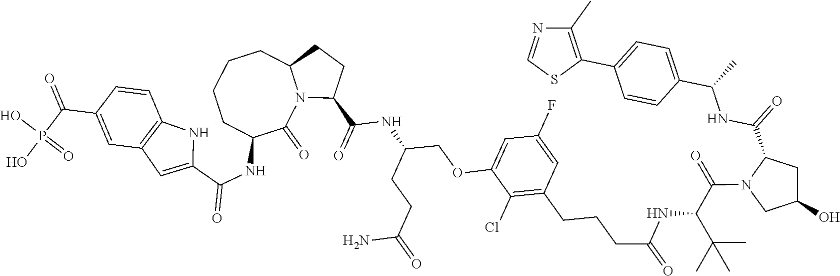 [SMILES: Cc1ncsc1-c1ccc([C@H](C)NC(=O)[C@@H]2C[C@@H](O)CN2C(=O)[C@@H](NC(=O)CCCc2cc(F)cc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)C(C)(C)C)cc1] |
| I-9 | 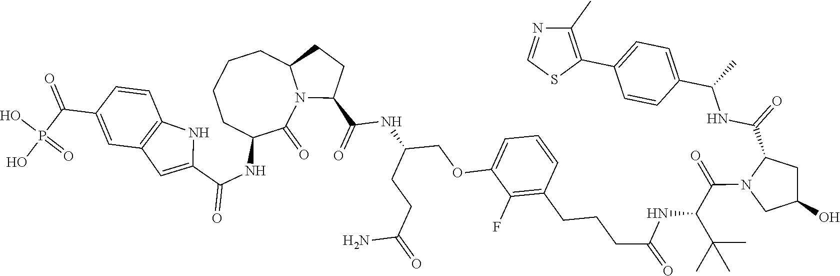 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2F)C(C)(C)C)cc1] |
| I- 10 | 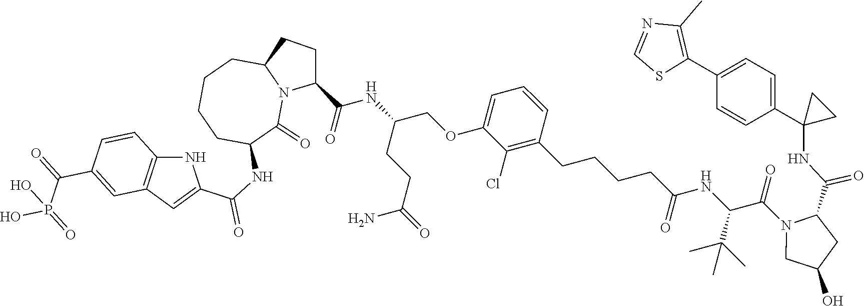 [SMILES: Cc1ncsc1-c1ccc(C2(NC(=O)[C@@H]3C[C@@H](O)CN3C(=O)[C@@H](NC(=O)CCCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)C(C)(C)C)CC2)cc1] |
| I- 11 | 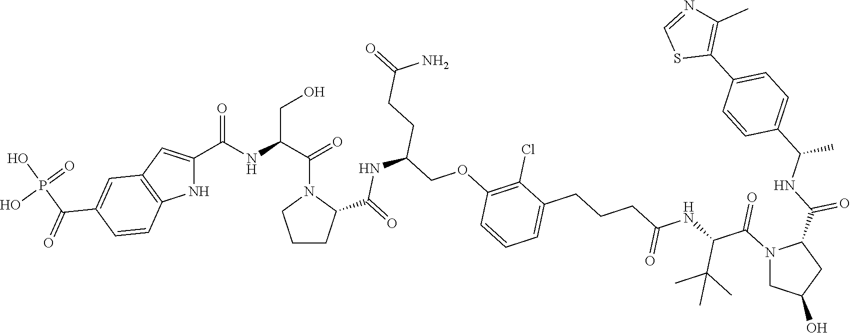 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@H](CO)NC(=O)c3cc4cc(C(=O)P(=O)(O)O)ccc4[nH]3)c2Cl)C(C)(C)C)cc1] |
| I- 12 | 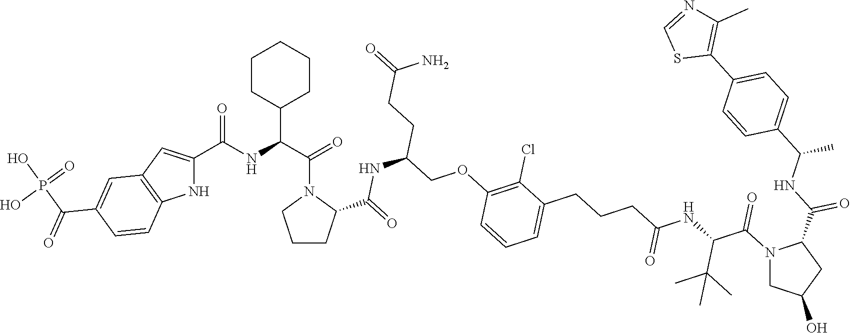 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@@H](NC(=O)c3cc4cc(C(=O)P(=O)(O)O)ccc4[nH]3)C3CCCCC3)c2Cl)C(C)(C)C)cc1] |
| I- 13 | 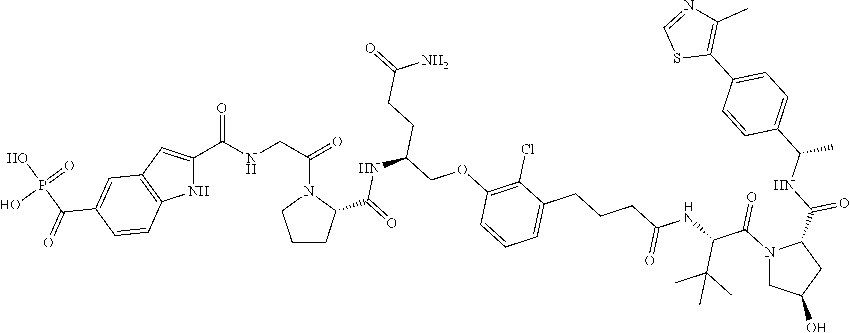 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)CNC(=O)c3cc4cc(C(=O)P(=O)(O)O)ccc4[nH]3)c2Cl)C(C)(C)C)cc1] |
| I- 14 | 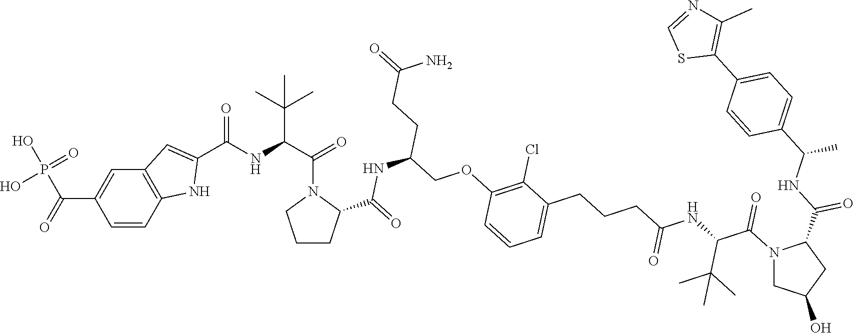 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@@H](NC(=O)c3cc4cc(C(=O)P(=O)(O)O)ccc4[nH]3)C(C)(C)C)c2Cl)C(C)(C)C)cc1] |
| I- 15 | 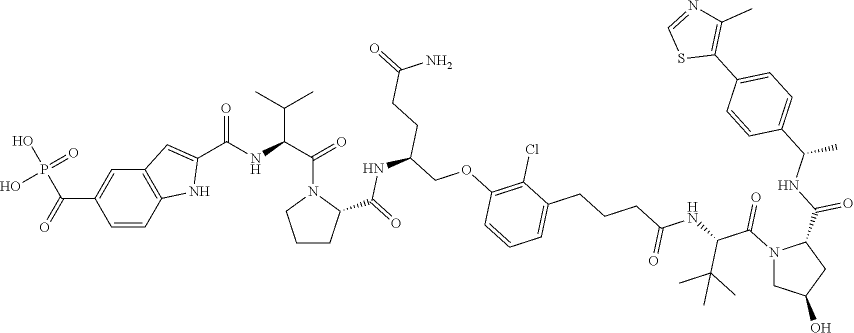 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@@H](NC(=O)c3cc4cc(C(=O)P(=O)(O)O)ccc4[nH]3)C(C)C)c2Cl)C(C)(C)C)cc1] |
| I- 20 | 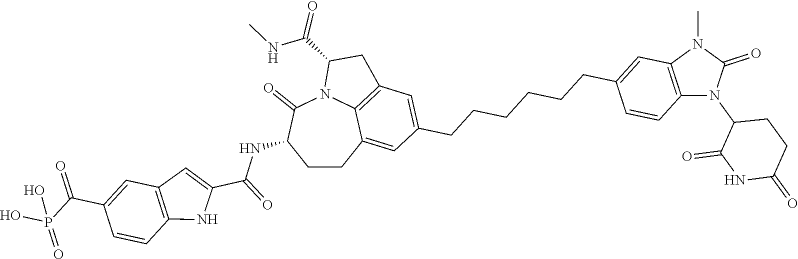 [SMILES: CNC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3] |
| I- 21 | 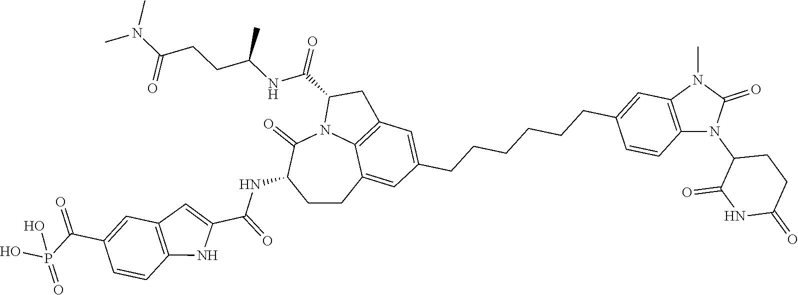 [SMILES: C[C@H](CCC(=O)N(C)C)NC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3] |
| I- 22 | 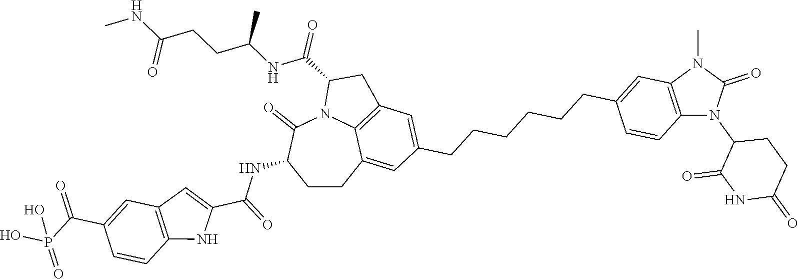 [SMILES: CNC(=O)CC[C@@H](C)NC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3] |
| I- 23 | 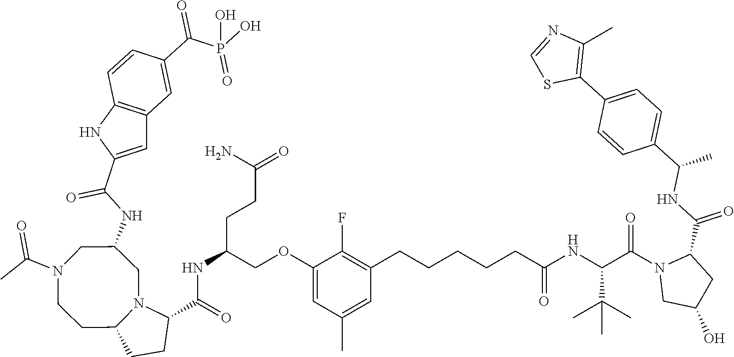 [SMILES: CC(=O)N1CC[C@H]2CC[C@@H](C(=O)N[C@@H](CCC(N)=O)COc3cc(C)cc(CCCCCC(=O)N[C@H](C(=O)N4C[C@@H](O)C[C@H]4C(=O)N[C@@H](C)c4ccc(-c5scnc5C)cc4)C(C)(C)C)c3F)N2C[C@@H](NC(=O)c2cc3cc(C(=O)P(=O)(O)O)ccc3[nH]2)C1] |
| I- 24 | 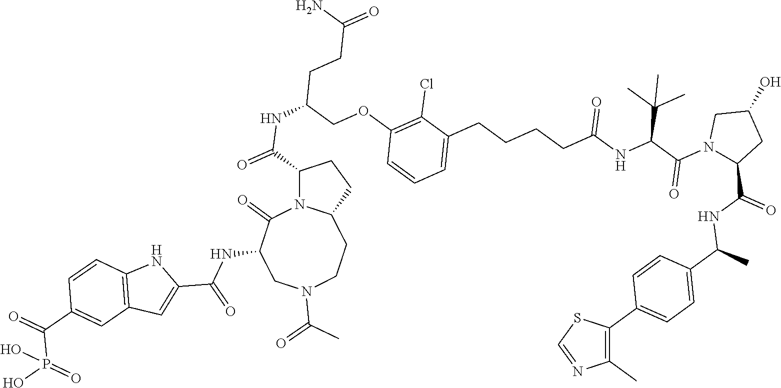 [SMILES: CC(=O)N1CC[C@H]2CC[C@@H](C(=O)N[C@H](CCC(N)=O)COc3cccc(CCCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 25 | 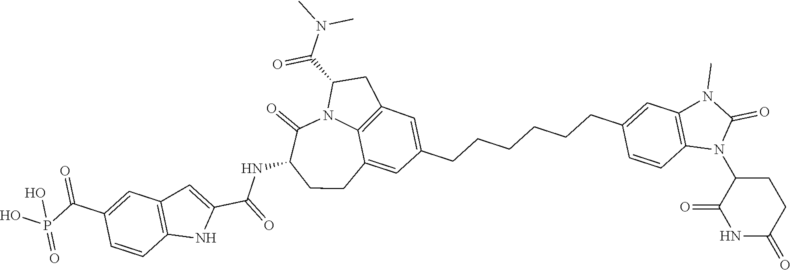 [SMILES: CN(C)C(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3] |
| I- 26 | 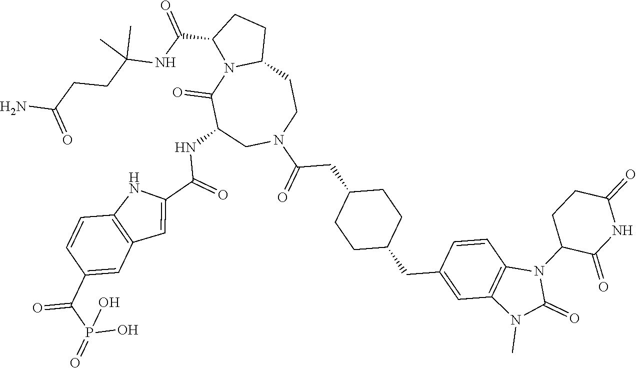 [SMILES: Cn1c(=O)n(C2CCC(=O)NC2=O)c2ccc(C[C@H]3CC[C@@H](CC(=O)N4CC[C@H]5CC[C@@H](C(=O)NC(C)(C)CCC(N)=O)N5C(=O)[C@@H](NC(=O)c5cc6cc(C(=O)P(=O)(O)O)ccc6[nH]5)C4)CC3)cc21] |
| I- 27 | 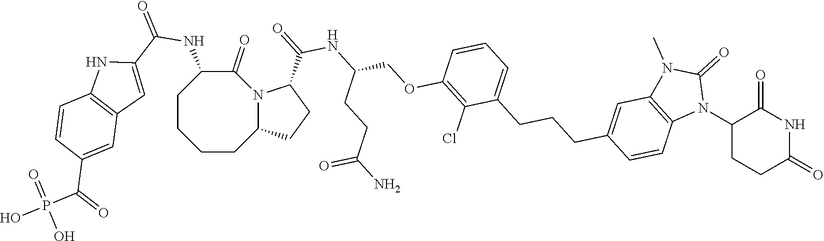 [SMILES: Cn1c(=O)n(C2CCC(=O)NC2=O)c2ccc(CCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21] |
| I- 28 | 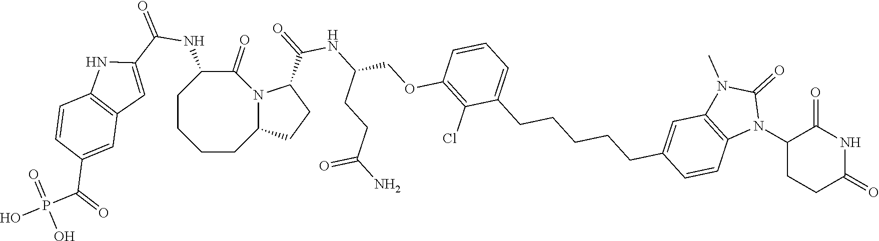 [SMILES: Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21] |
| I- 29 | 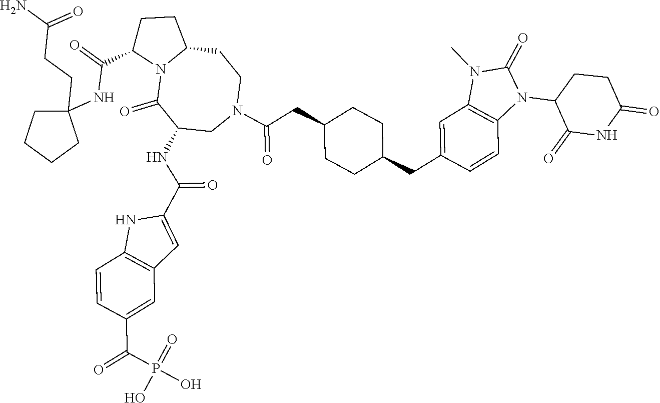 [SMILES: Cn1c(=O)n(C2CCC(=O)NC2=O)c2ccc(C[C@H]3CC[C@@H](CC(=O)N4CC[C@H]5CC[C@@H](C(=O)NC6(CCC(N)=O)CCCC6)N5C(=O)[C@@H](NC(=O)c5cc6cc(C(=O)P(=O)(O)O)ccc6[nH]5)C4)CC3)cc21] |
| I- 30 | 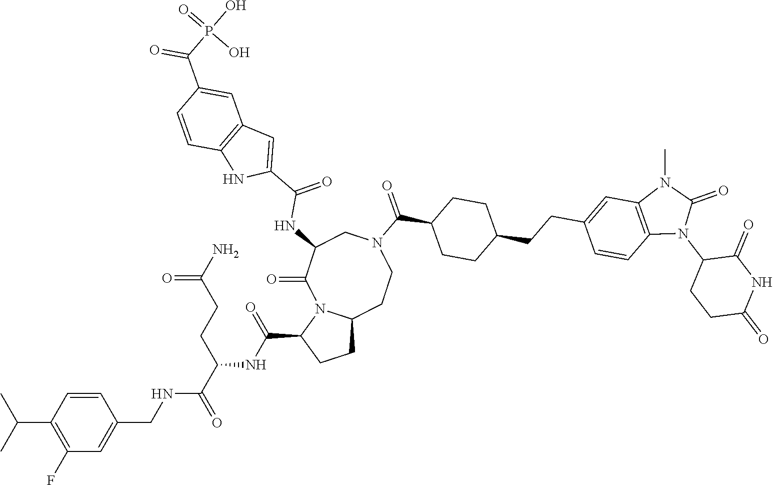 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1F] |
| I- 31 | 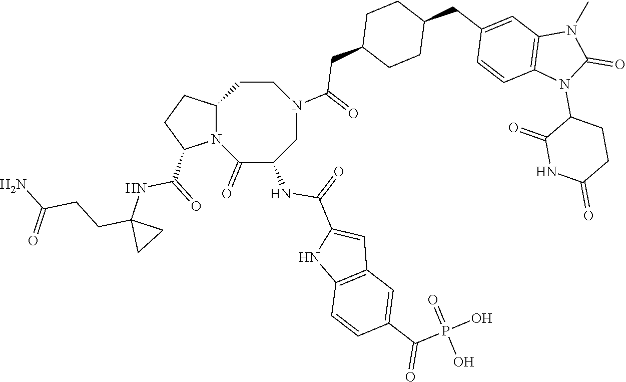 [SMILES: Cn1c(=O)n(C2CCC(=O)NC2=O)c2ccc(C[C@H]3CC[C@@H](CC(=O)N4CC[C@H]5CC[C@@H](C(=O)NC6(CCC(N)=O)CC6)N5C(=O)[C@@H](NC(=O)c5cc6cc(C(=O)P(=O)(O)O)ccc6[nH]5)C4)CC3)cc21] |
| I- 32 | 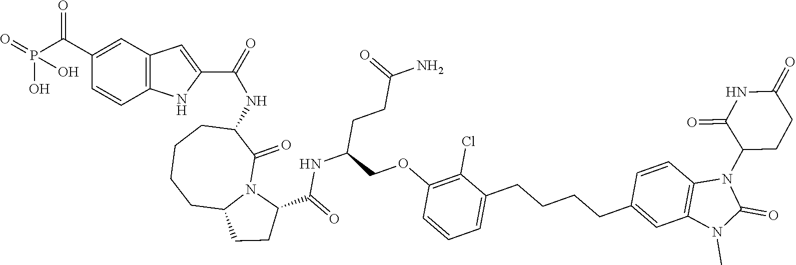 [SMILES: Cn1c(=O)n(C2CCC(=O)NC2=O)c2ccc(CCCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21] |
| I- 33 | 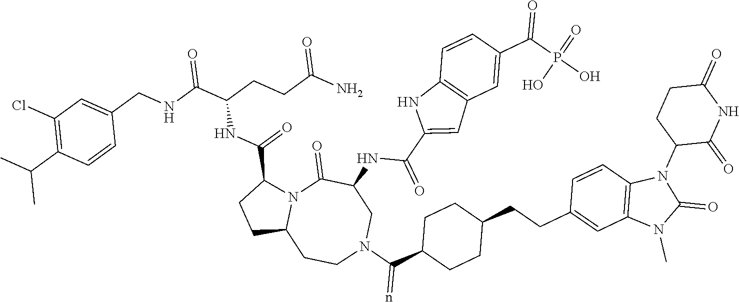 |
| I- 34 | 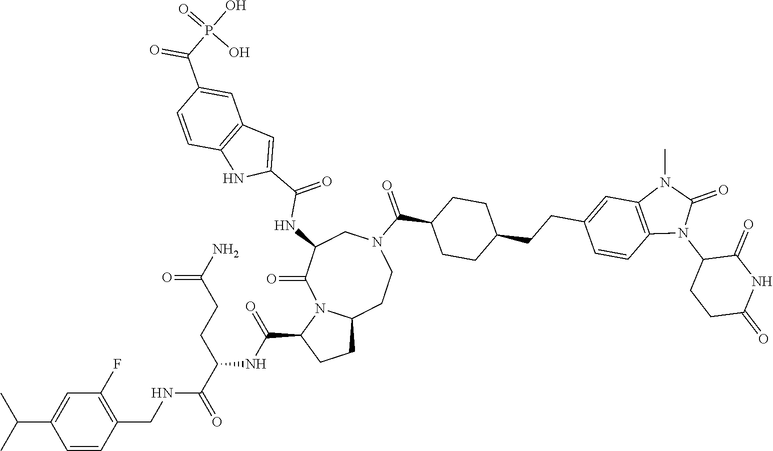 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)c(F)c1] |
| I- 35 | 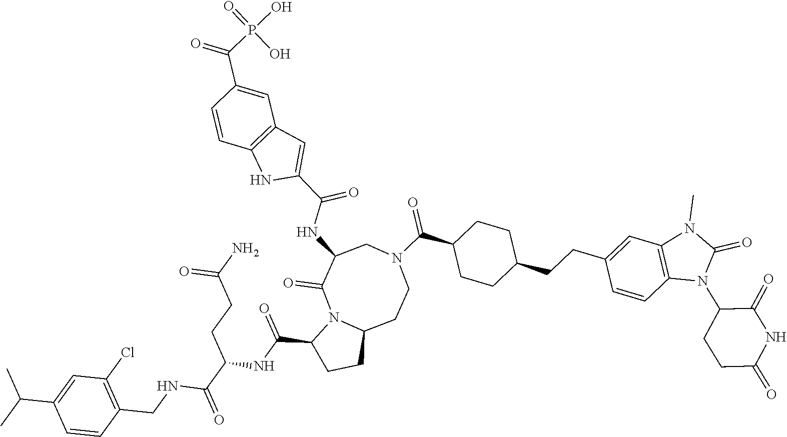 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)c(Cl)c1] |
| I- 36 | 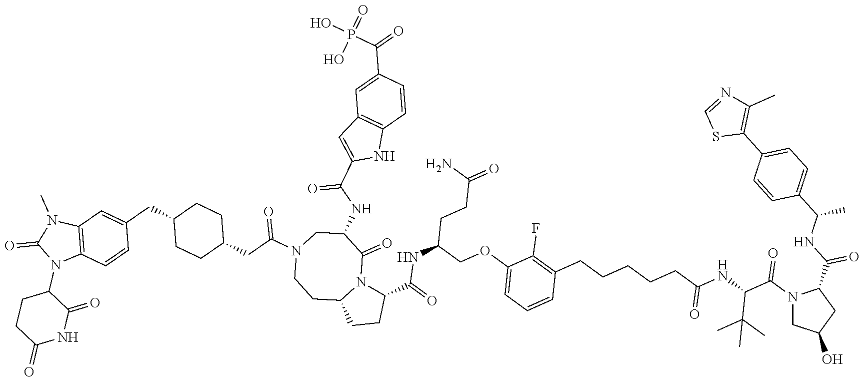 [SMILES: Cc1ncsc1-c1ccc([C@H](C)NC(=O)[C@@H]2C[C@@H](O)CN2C(=O)[C@@H](NC(=O)CCCCCc2cccc(OC[C@H](CCC(N)=O)NC(=O)[C@@H]3CC[C@@H]4CCN(C(=O)C[C@H]5CC[C@@H](Cc6ccc7c(c6)n(C)c(=O)n7C6CCC(=O)NC6=O)CC5)C[C@H](NC(=O)c5cc6cc(C(=O)P(=O)(O)O)ccc6[nH]5)C(=O)N43)c2F)C(C)(C)C)cc1] |
| I- 37 | 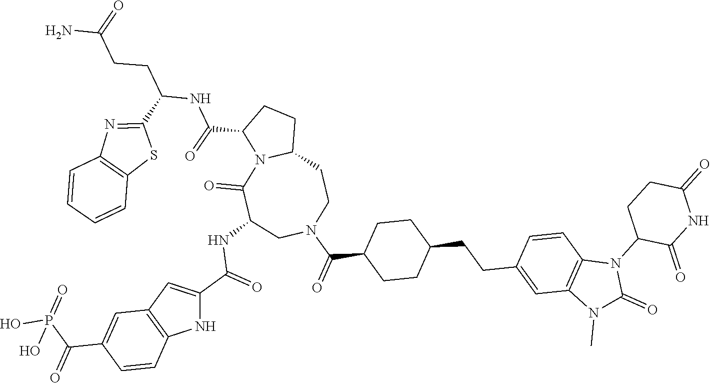 [SMILES: Cn1c(=O)n(C2CCC(=O)NC2=O)c2ccc(CC[C@H]3CC[C@@H](C(=O)N4CC[C@H]5CC[C@@H](C(=O)N[C@@H](CCC(N)=O)c6nc7ccccc7s6)N5C(=O)[C@@H](NC(=O)c5cc6cc(C(=O)P(=O)(O)O)ccc6[nH]5)C4)CC3)cc21] |
| I- 38 | 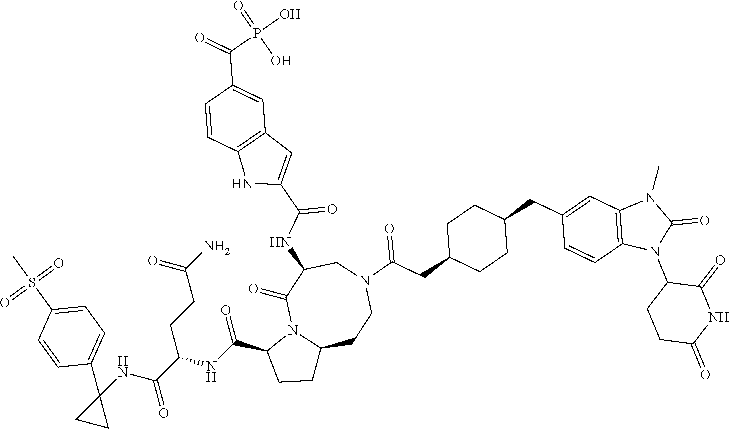 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)C(=O)NC6(c7ccc(S(C)(=O)=O)cc7)CC6)N5C(=O)[C@@H](NC(=O)c5cc6cc(C(=O)P(=O)(O)O)ccc6[nH]5)C4)CC3)cc21] |
| I- 39 | 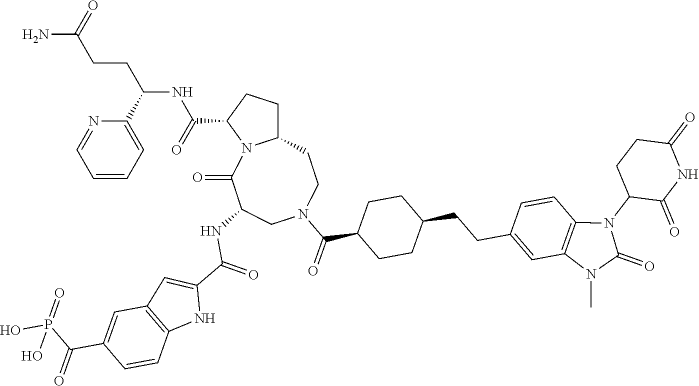 [SMILES: Cn1c(=O)n(C2CCC(=O)NC2=O)c2ccc(CC[C@H]3CC[C@@H](C(=O)N4CC[C@H]5CC[C@@H](C(=O)N[C@@H](CCC(N)=O)c6ccccn6)N5C(=O)[C@@H](NC(=O)c5cc6cc(C(=O)P(=O)(O)O)ccc6[nH]5)C4)CC3)cc21] |
| I- 40 | 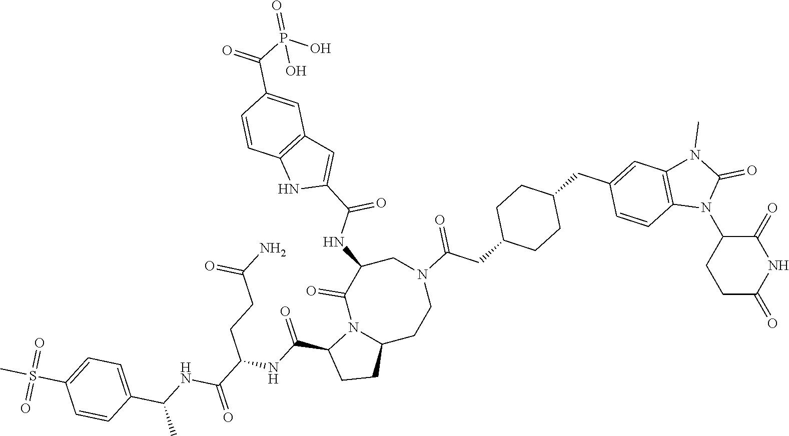 [SMILES: C[C@@H](NC(=O)[C@H](CCC(N)=O)NC(=O)[C@@H]1CC[C@@H]2CCN(C(=O)C[C@H]3CC[C@@H](Cc4ccc5c(c4)n(C)c(=O)n5C4CCC(=O)NC4=O)CC3)C[C@H](NC(=O)c3cc4cc(C(=O)P(=O)(O)O)ccc4[nH]3)C(=O)N21)c1ccc(S(C)(=O)=O)cc1] |
| I- 41 | 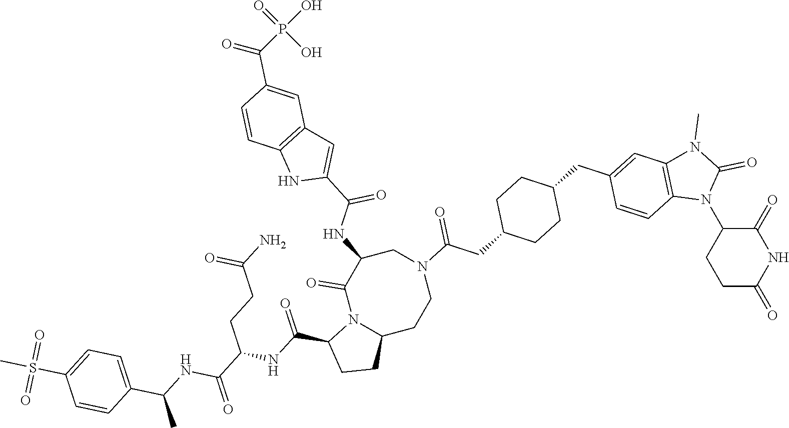 [SMILES: C[C@H](NC(=O)[C@H](CCC(N)=O)NC(=O)[C@@H]1CC[C@@H]2CCN(C(=O)C[C@H]3CC[C@@H](Cc4ccc5c(c4)n(C)c(=O)n5C4CCC(=O)NC4=O)CC3)C[C@H](NC(=O)c3cc4cc(C(=O)P(=O)(O)O)ccc4[nH]3)C(=O)N21)c1ccc(S(C)(=O)=O)cc1] |
| I- 42 | 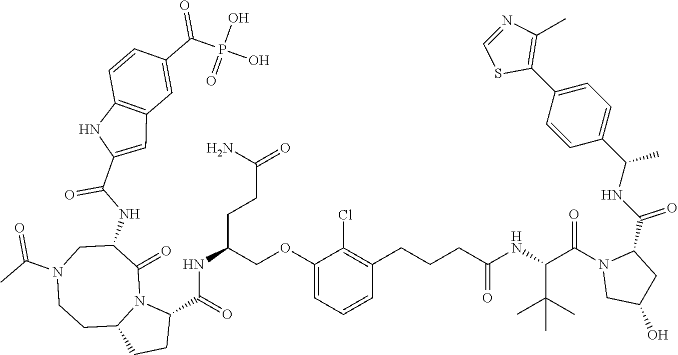 [SMILES: CC(=O)N1CC[C@H]2CC[C@@H](C(=O)N[C@@H](CCC(N)=O)COc3cccc(CCCC(=O)N[C@H](C(=O)N4C[C@@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 43 | 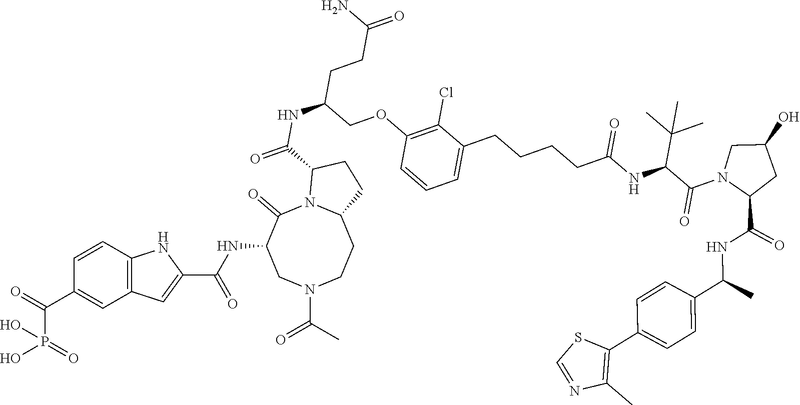 [SMILES: CC(=O)N1CC[C@H]2CC[C@@H](C(=O)N[C@@H](CCC(N)=O)COc3cccc(CCCCC(=O)N[C@H](C(=O)N4C[C@@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 44 | 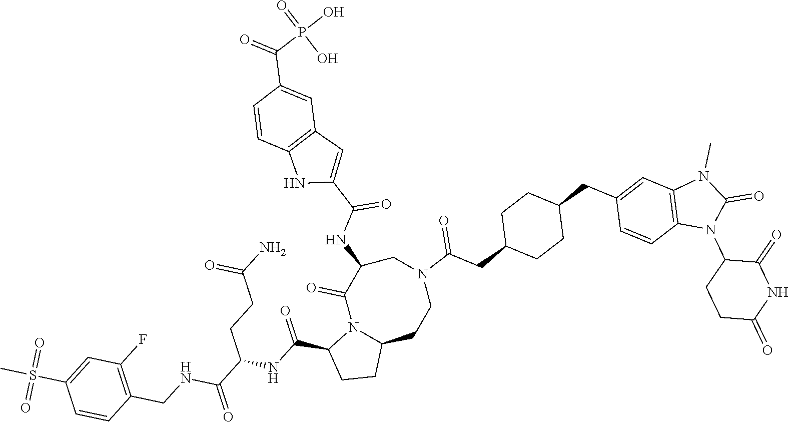 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)C(=O)NCc6ccc(S(C)(=O)=O)cc6F)N5C(=O)[C@@H](NC(=O)c5cc6cc(C(=O)P(=O)(O)O)ccc6[nH]5)C4)CC3)cc21] |
| I- 45 | 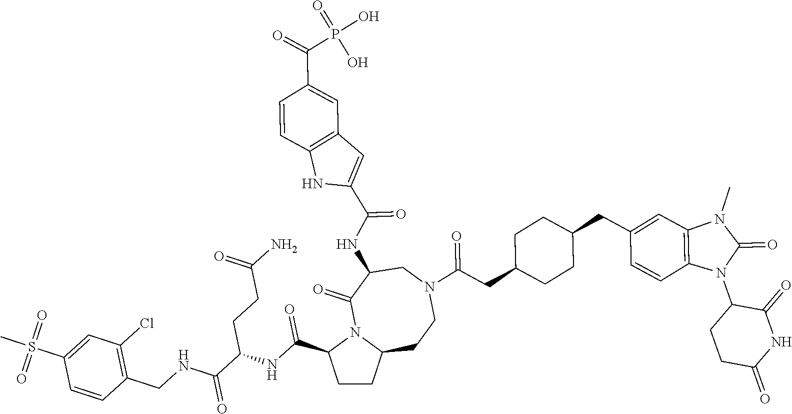 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)C(=O)NCc6ccc(S(C)(=O)=O)cc6Cl)N5C(=O)[C@@H](NC(=O)c5cc6cc(C(=O)P(=O)(O)O)ccc6[nH]5)C4)CC3)cc21] |
| I- 46 | 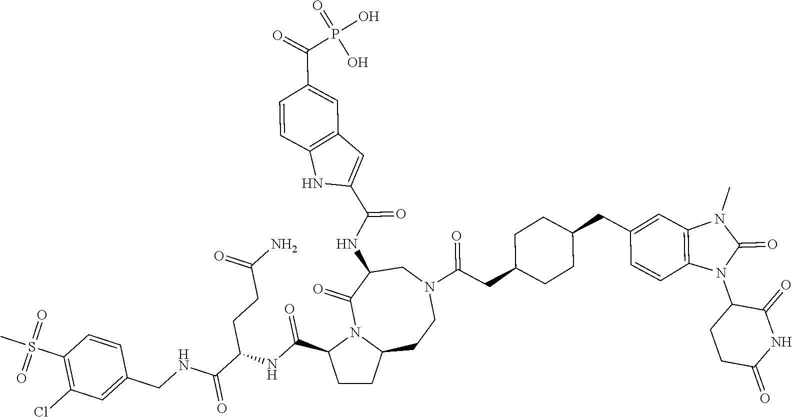 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)C(=O)NCc6ccc(S(C)(=O)=O)c(Cl)c6)N5C(=O)[C@@H](NC(=O)c5cc6cc(C(=O)P(=O)(O)O)ccc6[nH]5)C4)CC3)cc21] |
| I- 47 | 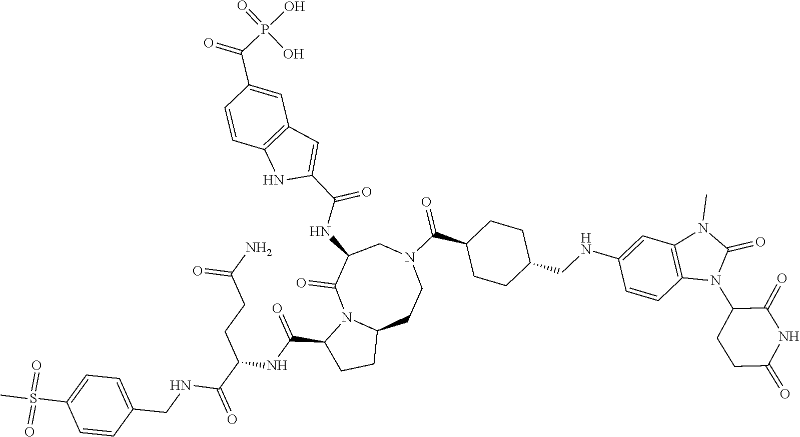 [SMILES: Cn1c(=O)n(C2CCC(=O)NC2=O)c2ccc(NC[C@H]3CC[C@H](C(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 48 | 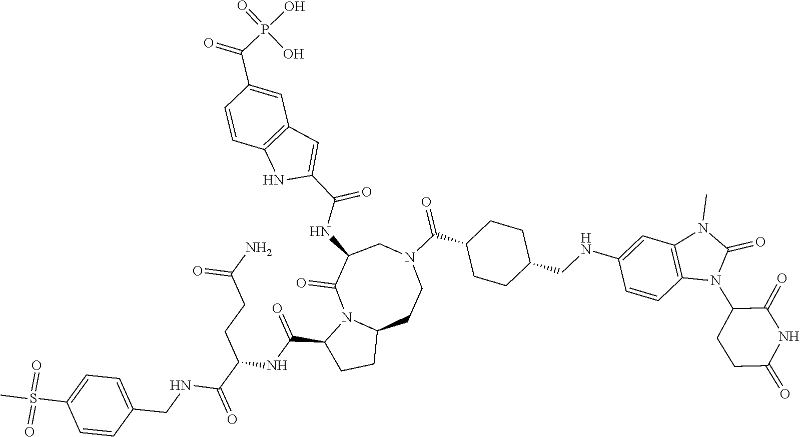 [SMILES: Cn1c(=O)n(C2CCC(=O)NC2=O)c2ccc(NC[C@H]3CC[C@@H](C(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 49 | 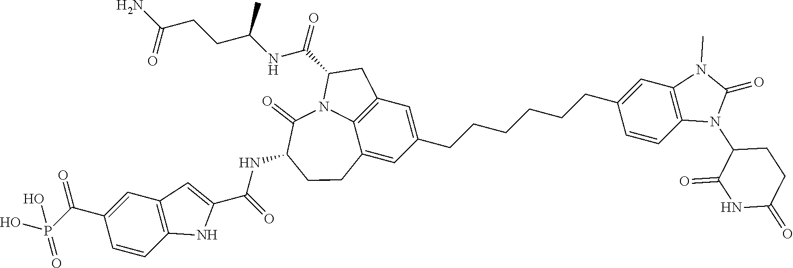 [SMILES: C[C@H](CCC(N)=O)NC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3] |
| I- 50 | 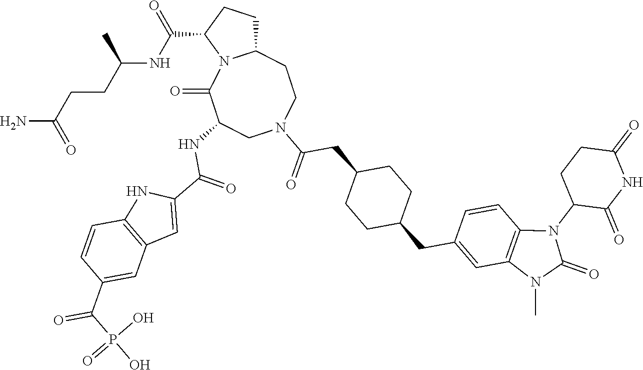 [SMILES: C[C@H](CCC(N)=O)NC(=O)[C@@H]1CC[C@@H]2CCN(C(=O)C[C@H]3CC[C@@H](Cc4ccc5c(c4)n(C)c(=O)n5C4CCC(=O)NC4=O)CC3)C[C@H](NC(=O)c3cc4cc(C(=O)P(=O)(O)O)ccc4[nH]3)C(=O)N21] |
| I- 51 | 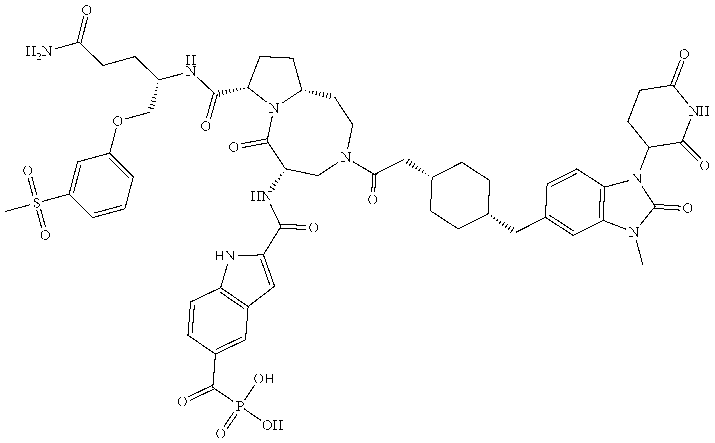 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)COc6cccc(S(C)(=O)=O)c6)N5C(=O)[C@@H](NC(=O)c5cc6cc(C(=O)P(=O)(O)O)ccc6[nH]5)C4)CC3)cc21] |
| I- 52 | 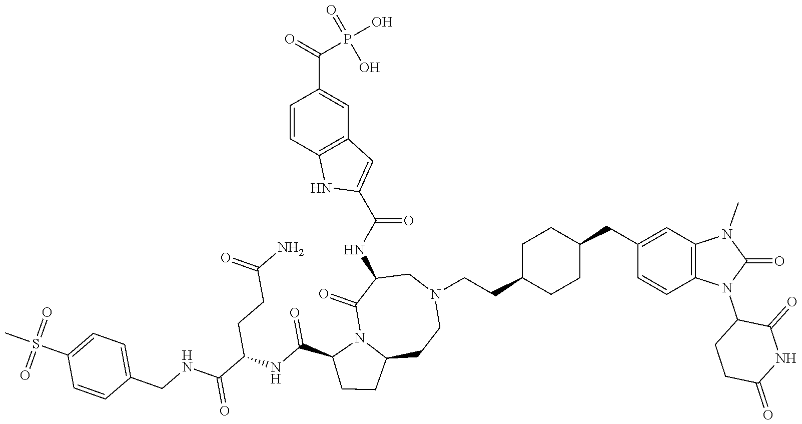 [SMILES: Cn1c(=O)n(C2CCC(=O)NC2=O)c2ccc(C[C@H]3CC[C@@H](CCN4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 53 | 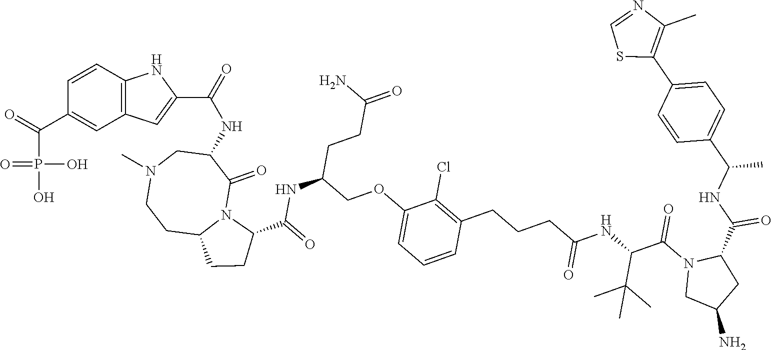 [SMILES: Cc1ncsc1-c1ccc([C@H](C)NC(=O)[C@@H]2C[C@@H](N)CN2C(=O)[C@@H](NC(=O)CCCc2cccc(OC[C@H](CCC(N)=O)NC(=O)[C@@H]3CC[C@@H]4CCN(C)C[C@H](NC(=O)c5cc6cc(C(=O)P(=O)(O)O)ccc6[nH]5)C(=O)N43)c2Cl)C(C)(C)C)cc1] |
| I- 54 | 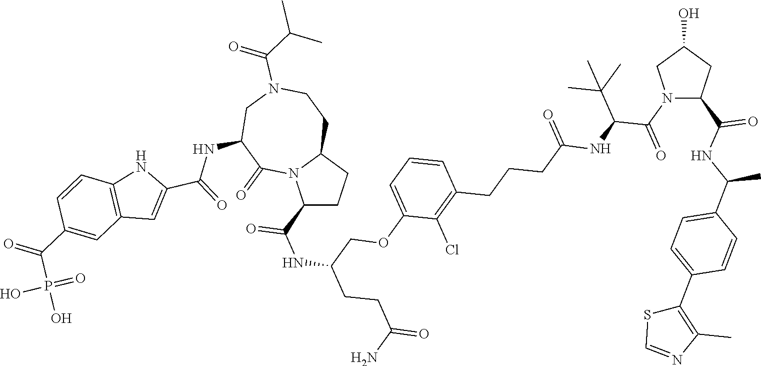 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCN(C(=O)C(C)C)C[C@H](NC(=O)c5cc6cc(C(=O)P(=O)(O)O)ccc6[nH]5)C(=O)N43)c2Cl)C(C)(C)C)cc1] |
| I- 55 | 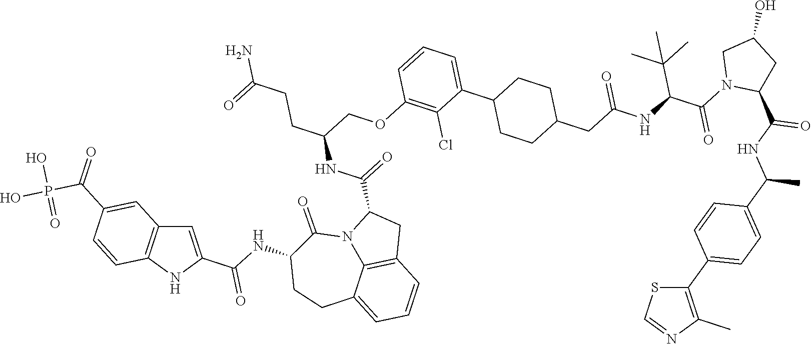 [SMILES: Cc1ncsc1-c1ccc([C@H](C)NC(=O)[C@@H]2C[C@@H](O)CN2C(=O)[C@@H](NC(=O)CC2CCC(c3cccc(OC[C@H](CCC(N)=O)NC(=O)[C@@H]4Cc5cccc6c5N4C(=O)[C@@H](NC(=O)c4cc5cc(C(=O)P(=O)(O)O)ccc5[nH]4)CC6)c3Cl)CC2)C(C)(C)C)cc1] |
| I- 56 | 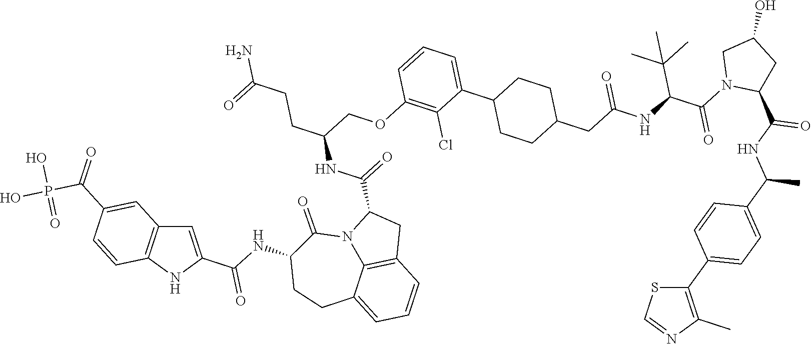 [SMILES: Cc1ncsc1-c1ccc([C@H](C)NC(=O)[C@@H]2C[C@@H](O)CN2C(=O)[C@@H](NC(=O)CC2CCC(c3cccc(OC[C@H](CCC(N)=O)NC(=O)[C@@H]4Cc5cccc6c5N4C(=O)[C@@H](NC(=O)c4cc5cc(C(=O)P(=O)(O)O)ccc5[nH]4)CC6)c3Cl)CC2)C(C)(C)C)cc1] |
| I- 57 | 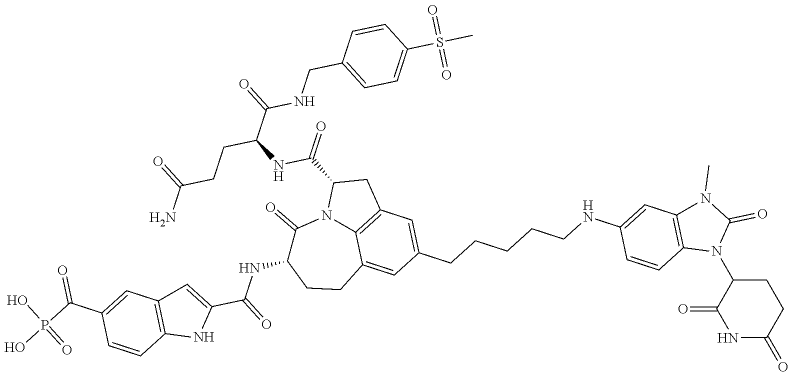 [SMILES: Cn1c(=O)n(C2CCC(=O)NC2=O)c2ccc(NCCCCCc3cc4c5c(c3)C[C@@H](C(=O)N[C@@H](CCC(N)=O)C(=O)NCc3ccc(S(C)(=O)=O)cc3)N5C(=O)[C@@H](NC(=O)c3cc5cc(C(=O)P(=O)(O)O)ccc5[nH]3)CC4)cc21] |
| I- 58 | 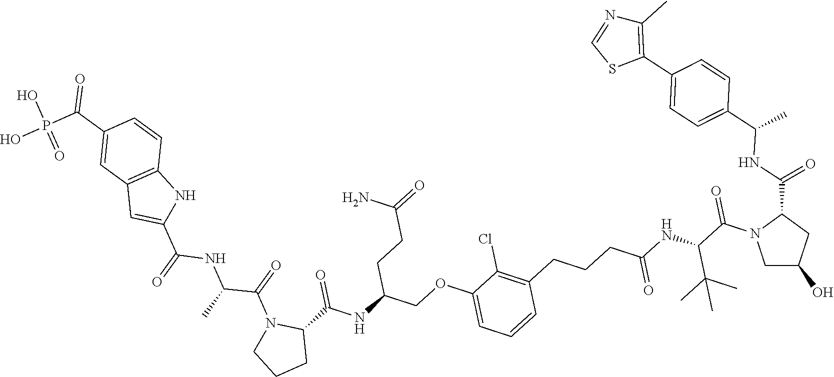 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@H](C)NC(=O)c3cc4cc(C(=O)P(=O)(O)O)ccc4[nH]3)c2Cl)C(C)(C)C)cc1] |
| I- 59 | 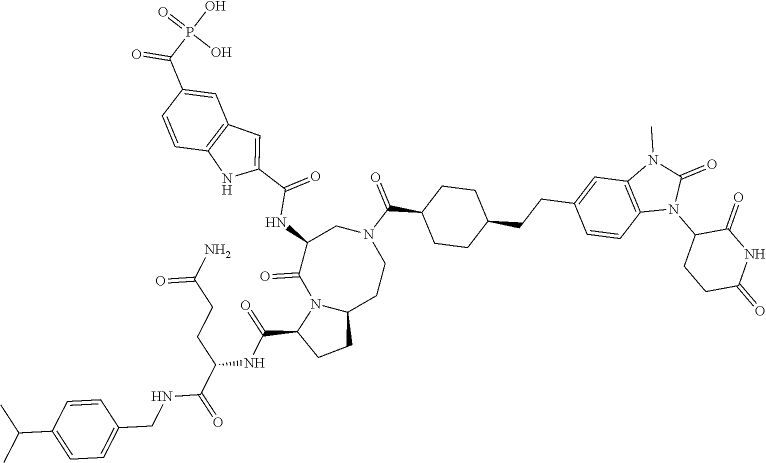 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1] |
| I- 60 | 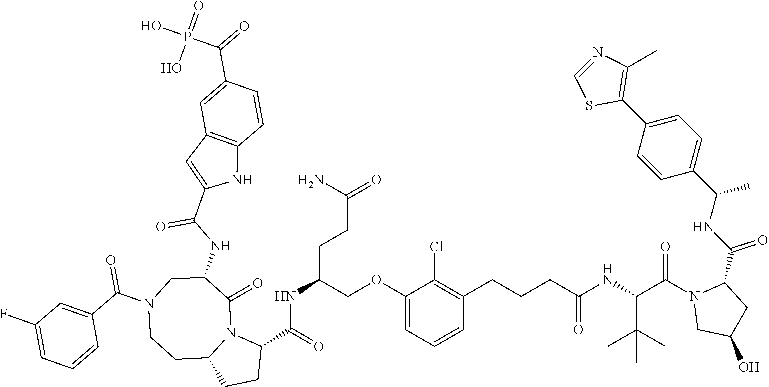 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCN(C(=O)c5cccc(F)c5)C[C@H](NC(=O)c5cc6cc(C(=O)P(=O)(O)O)ccc6[nH]5)C(=O)N43)c2Cl)C(C)(C)C)cc1] |
| I- 61 | 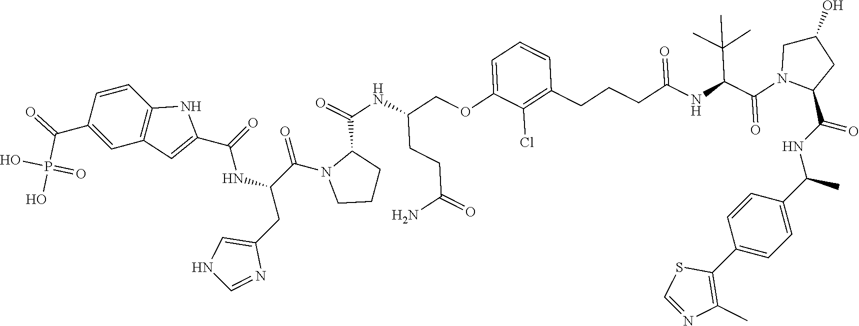 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@H](Cc3c[nH]cn3)NC(=O)c3cc4cc(C(=O)P(=O)(O)O)ccc4[nH]3)c2Cl)C(C)(C)C)cc1] |
| I- 62 | 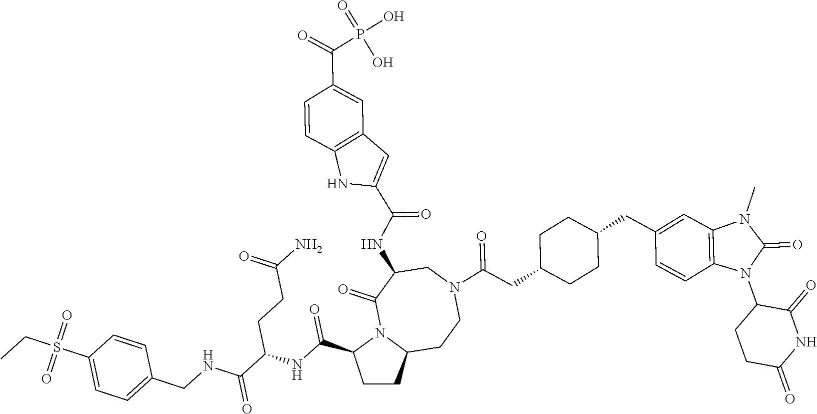 [SMILES: CCS(=O)(=O)c1ccc(CNC(=O)[C@H](CCC(N)=O)NC(=O)[C@@H]2CC[C@@H]3CCN(C(=O)C[C@H]4CC[C@@H](Cc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1] |
| I- 63 | 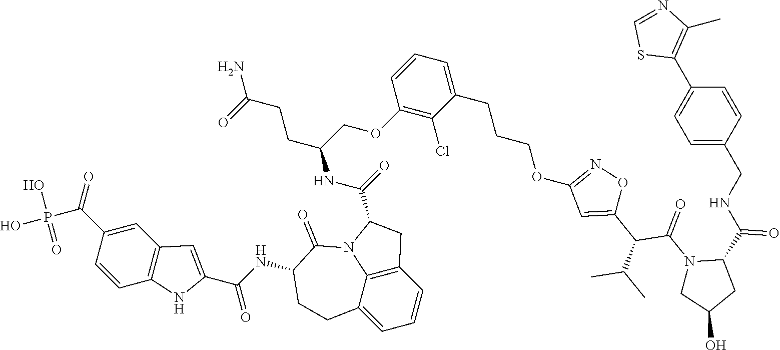 [SMILES: Cc1ncsc1-c1ccc(CNC(=O)[C@@H]2C[C@@H](O)CN2C(=O)[C@@H](c2cc(OCCCc3cccc(OC[C@H](CCC(N)=O)NC(=O)[C@@H]4Cc5cccc6c5N4C(=O)[C@@H](NC(=O)c4cc5cc(C(=O)P(=O)(O)O)ccc5[nH]4)CC6)c3Cl)no2)C(C)C)cc1] |
| I- 64 | 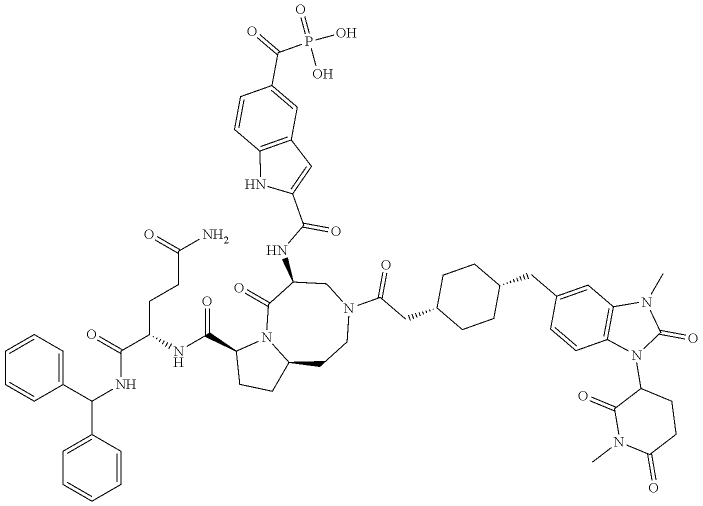 [SMILES: CN1C(=O)CCC(n2c(=O)n(C)c3cc(C[C@H]4CC[C@@H](CC(=O)N5CC[C@H]6CC[C@@H](C(=O)N[C@@H](CCC(N)=O)C(=O)NC(c7ccccc7)c7ccccc7)N6C(=O)[C@@H](NC(=O)c6cc7cc(C(=O)P(=O)(O)O)ccc7[nH]6)C5)CC4)ccc32)C1=O] |
| I- 65 | 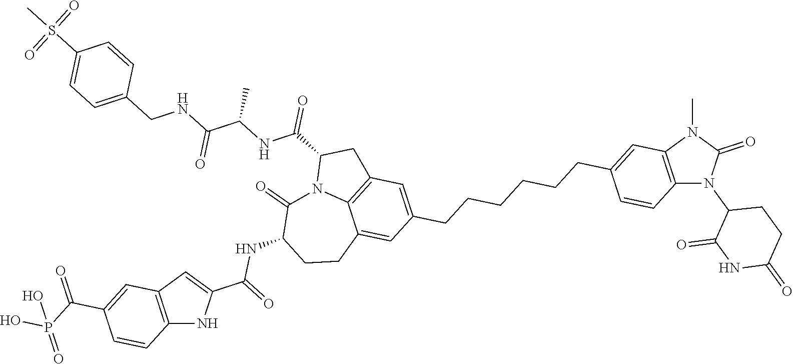 [SMILES: C[C@H](NC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3)C(=O)NCc1ccc(S(C)(=O)=O)cc1] |
| I- 66 | 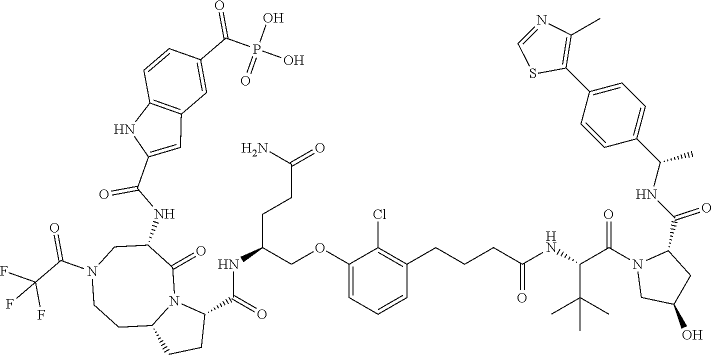 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCN(C(=O)C(F)(F)F)C[C@H](NC(=O)c5cc6cc(C(=O)P(=O)(O)O)ccc6[nH]5)C(=O)N43)c2Cl)C(C)(C)C)cc1] |
| I- 67 | 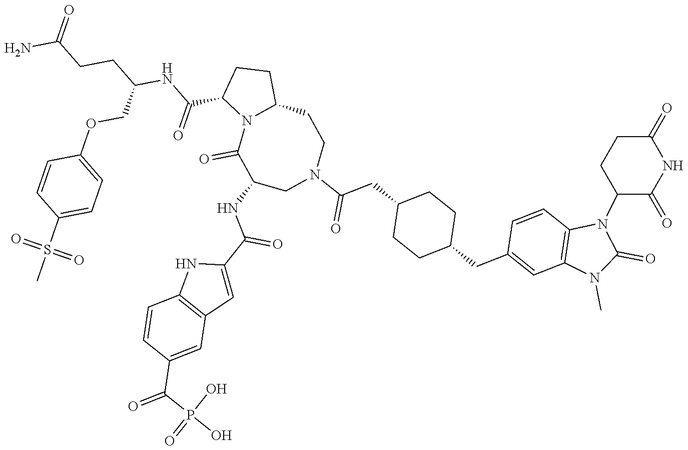 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)COc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 68 | 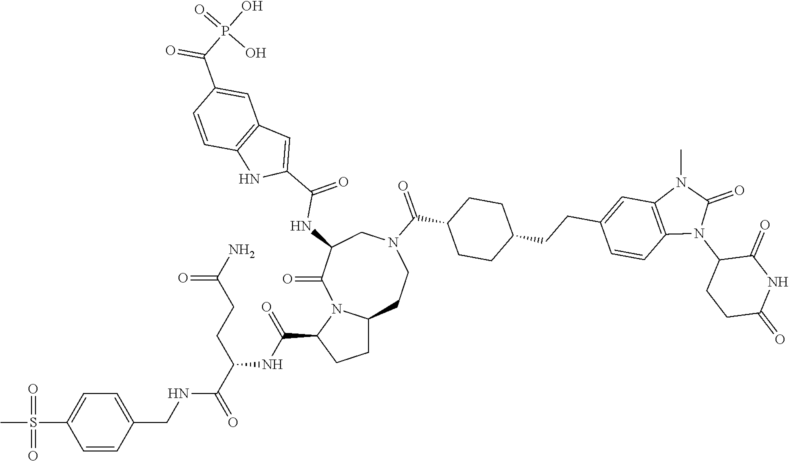 [SMILES: Cn1c(=O)n(C2CCC(=O)NC2=O)c2ccc(CC[C@H]3CC[C@@H](C(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 69 | 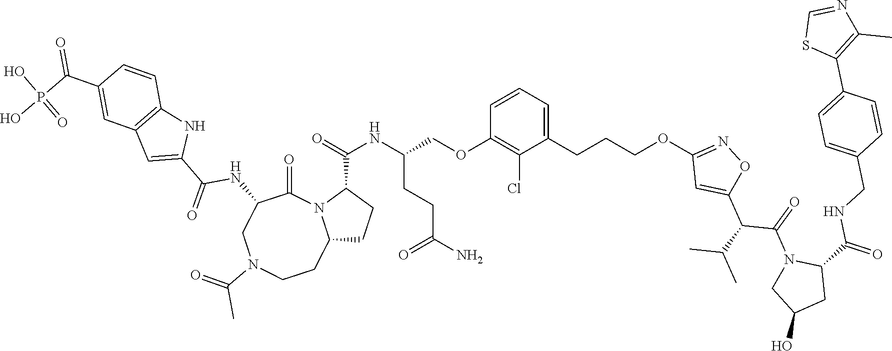 [SMILES: CC(=O)N1CC[C@H]2CC[C@@H](C(=O)N[C@@H](CCC(N)=O)COc3cccc(CCCOc4cc([C@H](C(=O)N5C[C@H](O)C[C@H]5C(=O)NCc5ccc(-c6scnc6C)cc5)C(C)C)on4)c3Cl)N2C(=O)[C@@H](NC(=O)c2cc3cc(C(=O)P(=O)(O)O)ccc3[nH]2)C1] |
| I- 70 | 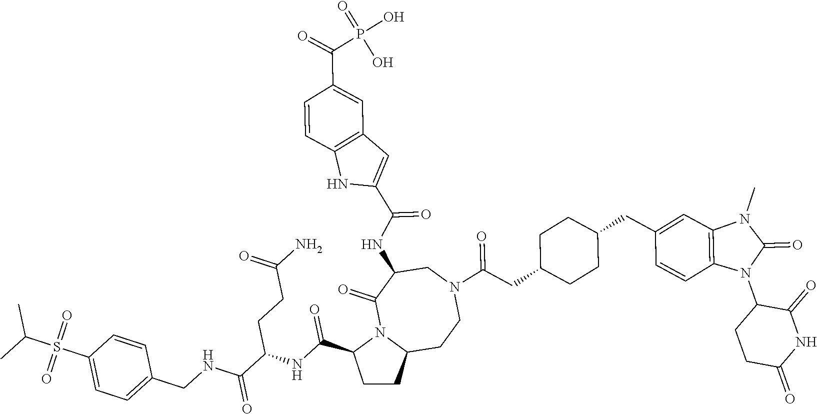 [SMILES: CC(C)S(=O)(=O)c1ccc(CNC(=O)[C@H](CCC(N)=O)NC(=O)[C@@H]2CC[C@@H]3CCN(C(=O)C[C@H]4CC[C@@H](Cc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1] |
| I- 71 | 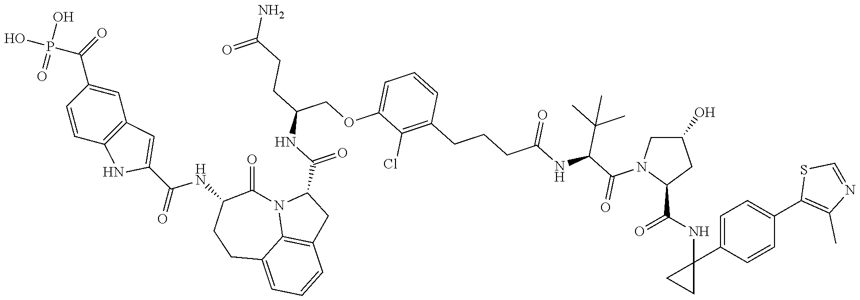 [SMILES: Cc1ncsc1-c1ccc(C2(NC(=O)[C@@H]3C[C@@H](O)CN3C(=O)[C@@H](NC(=O)CCCc3cccc(OC[C@H](CCC(N)=O)NC(=O)[C@@H]4Cc5cccc6c5N4C(=O)[C@@H](NC(=O)c4cc5cc(C(=O)P(=O)(O)O)ccc5[nH]4)CC6)c3Cl)C(C)(C)C)CC2)cc1] |
| I- 72 | 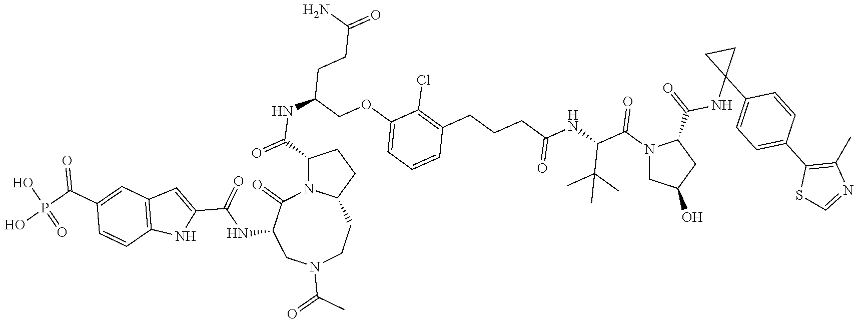 [SMILES: CC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)NC4(c5ccc(-c6scnc6C)cc5)CC4)C(C)(C)C)c3Cl)N2C(=O)[C@@H](NC(=O)c2cc3cc(C(=O)P(=O)(O)O)ccc3[nH]2)C1] |
| I- 73 | 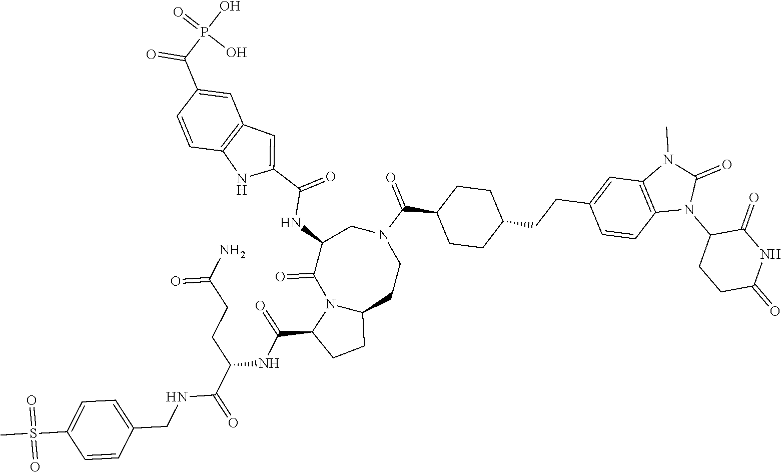 [SMILES: Cn1c(=O)n(C2CCC(=O)NC2=O)c2ccc(CC[C@H]3CC[C@H](C(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 74 | 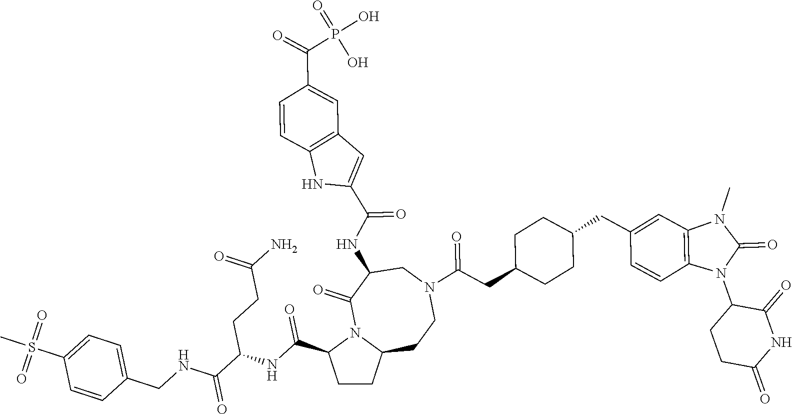 [SMILES: Cn1c(=O)n(C2CCC(=O)NC2=O)c2ccc(C[C@H]3CC[C@H](CC(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21] |
| I- 75 | 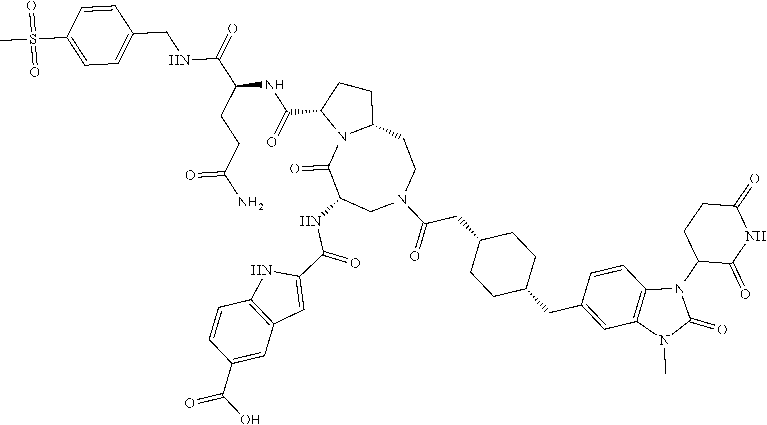 [SMILES: Cn1c(=O)n(C2CCC(=O)NC2=O)c2ccc(C[C@H]3CC[C@@H](CC(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)O)ccc6[nH]5)C4)CC3)cc21] |
| I- 76 | 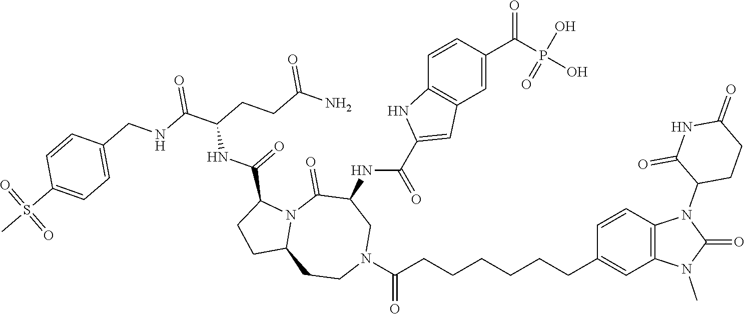 [SMILES: Cn1c(=O)n(C2CCC(=O)NC2=O)c2ccc(CCCCCCC(=O)N3CC[C@H]4CC[C@@H](C(=O)N[C@@H](CCC(N)=O)C(=O)NCc5ccc(S(C)(=O)=O)cc5)N4C(=O)[C@@H](NC(=O)c4cc5cc(C(=O)P(=O)(O)O)ccc5[nH]4)C3)cc21] |
| I- 77 | 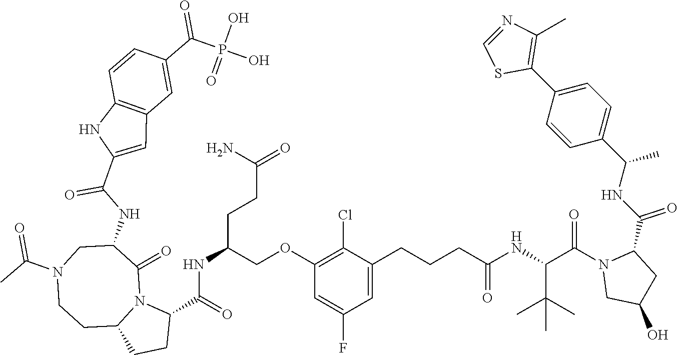 [SMILES: CC(=O)N1CC[C@H]2CC[C@@H](C(=O)N[C@@H](CCC(N)=O)COc3cc(F)cc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 78 | 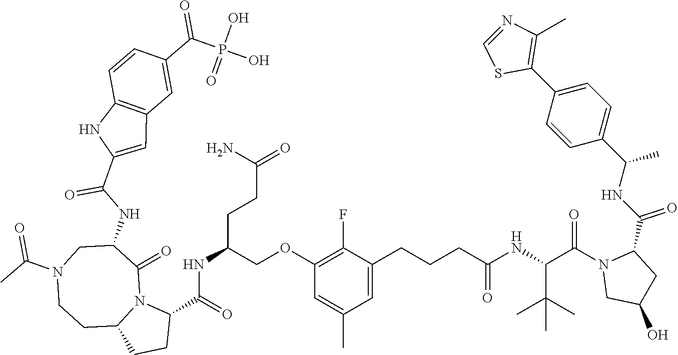 [SMILES: CC(=O)N1CC[C@H]2CC[C@@H](C(=O)N[C@@H](CCC(N)=O)COc3cc(C)cc(CCCC(=O)N[C@H](C(=O)N4C[C@H](O)C[C@H]4C(=O)N[C@@H](C)c4ccc(-c5scnc5C)cc4)C(C)(C)C)c3F)N2C(=O)[C@@H](NC(=O)c2cc3cc(C(=O)P(=O)(O)O)ccc3[nH]2)C1] |
| I- 79 | 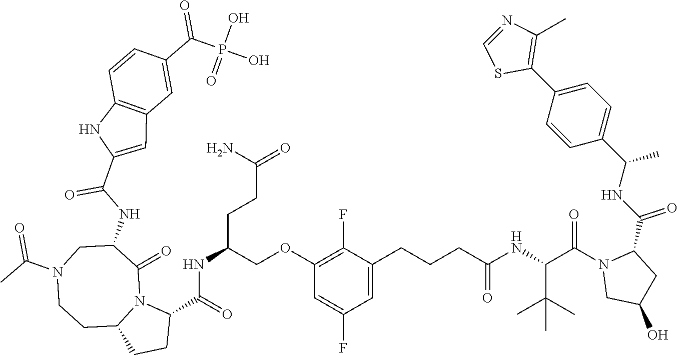 [SMILES: CC(=O)N1CC[C@H]2CC[C@@H](C(=O)N[C@@H](CCC(N)=O)COc3cc(F)cc(CCCC(=O)N[C@H](C(=O)N4C[C@H](O)C[C@H]4C(=O)N[C@@H](C)c4ccc(-c5scnc5C)cc4)C(C)(C)C)c3F)N2C(=O)[C@@H](NC(=O)c2cc3cc(C(=O)P(=O)(O)O)ccc3[nH]2)C1] |
| I- 80 | 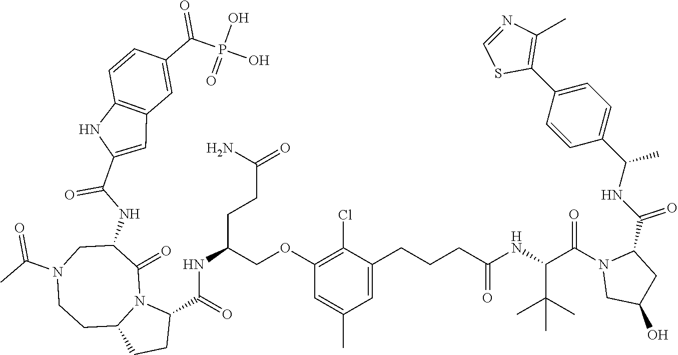 [SMILES: CC(=O)N1CC[C@H]2CC[C@@H](C(=O)N[C@@H](CCC(N)=O)COc3cc(C)cc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 81 | 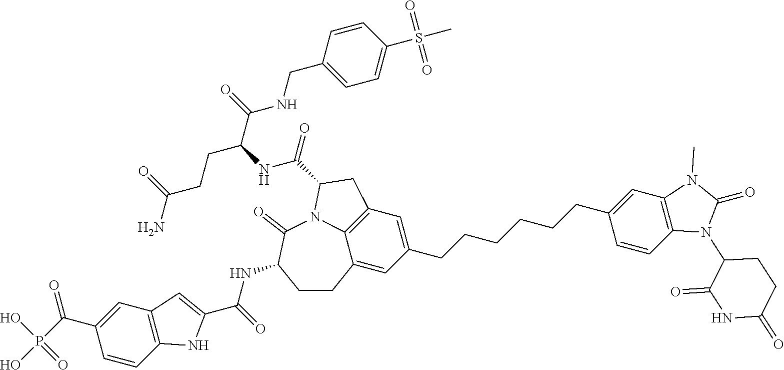 [SMILES: Cn1c(=O)n(C2CCC(=O)NC2=O)c2ccc(CCCCCCc3cc4c5c(c3)C[C@@H](C(=O)N[C@@H](CCC(N)=O)C(=O)NCc3ccc(S(C)(=O)=O)cc3)N5C(=O)[C@@H](NC(=O)c3cc5cc(C(=O)P(=O)(O)O)ccc5[nH]3)CC4)cc21] |
| I- 82 | 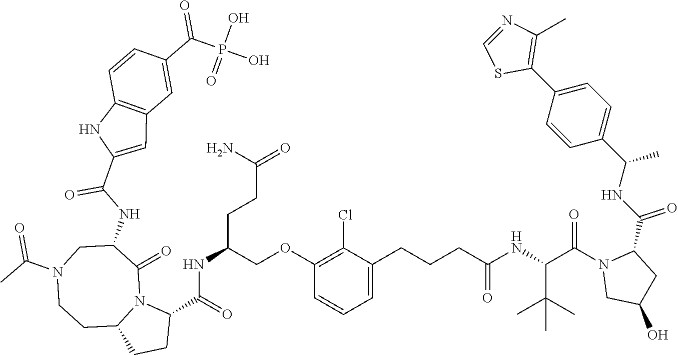 [SMILES: CC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 83 | 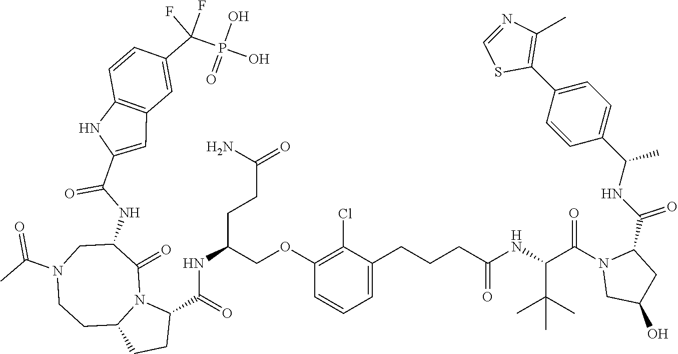 [SMILES: CC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(F)(F)P(=O)(O)O)ccc3[nH]2)C1] |
| I- 84 | 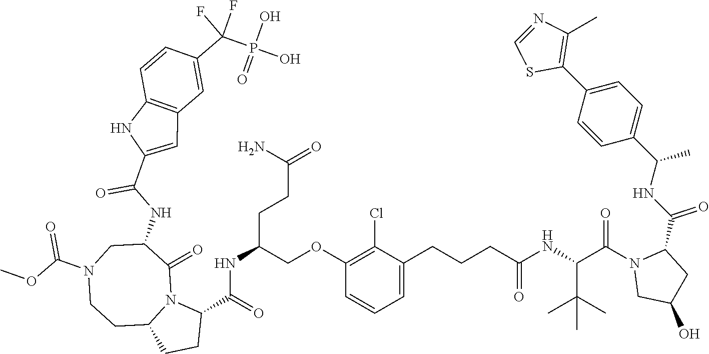 [SMILES: COC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(F)(F)P(=O)(O)O)ccc3[nH]2)C1] |
| I- 85 | 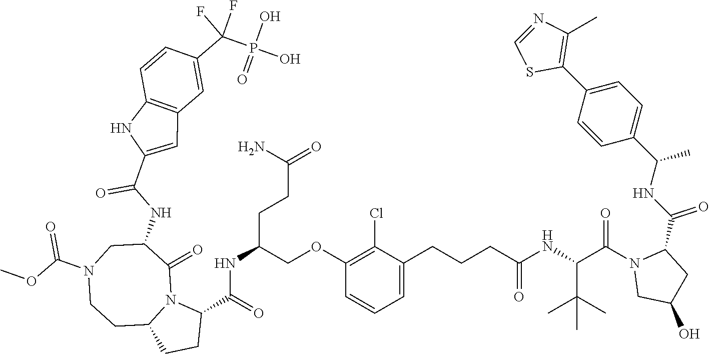 [SMILES: COC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(F)(F)P(=O)(O)O)ccc3[nH]2)C1] |
| I- 86 | 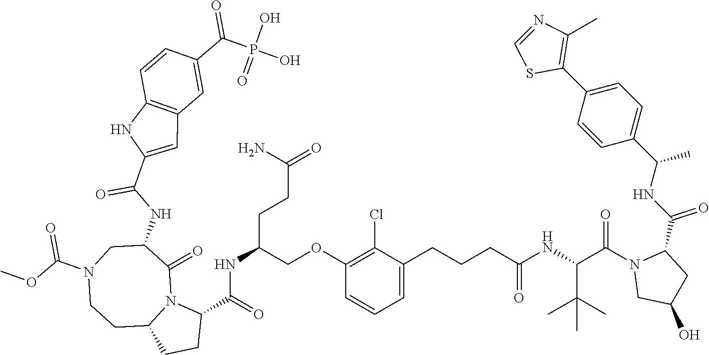 [SMILES: COC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 87 | 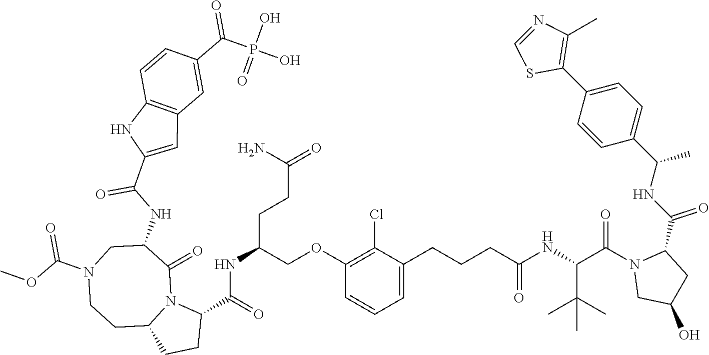 [SMILES: COC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1] |
| I- 88 | 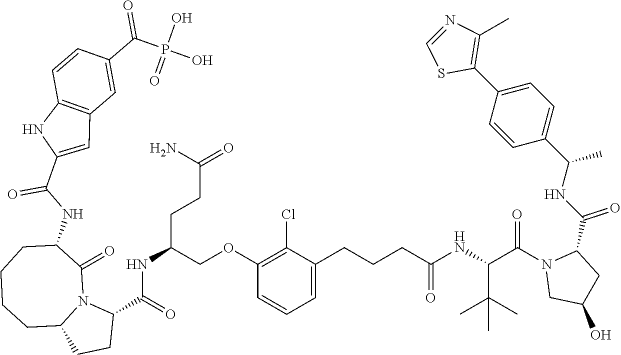 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)C(C)(C)C)cc1] |
| I- 89 | 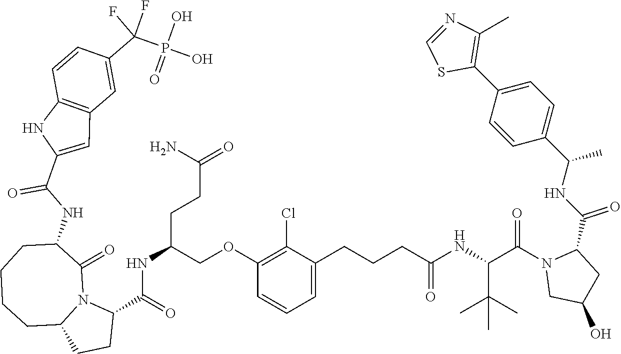 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CC[C@@H]4CCCC[C@H](NC(=O)c5cc6cc(C(F)(F)P(=O)(O)O)ccc6[nH]5)C(=O)N43)c2Cl)C(C)(C)C)cc1] |
| I- 90 | 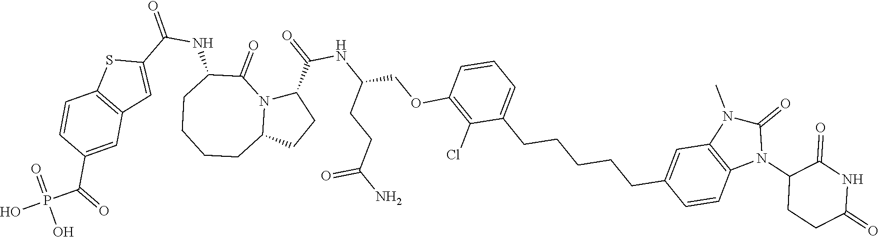 [SMILES: Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(=O)P(=O)(O)O)ccc7s6)C(=O)N54)c3Cl)cc21] |
| I- 91 | 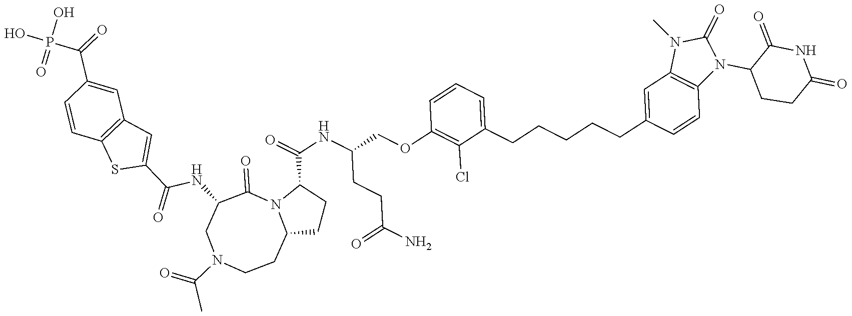 [SMILES: CC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(=O)P(=O)(O)O)ccc3s2)C1] |
| I- 92 | 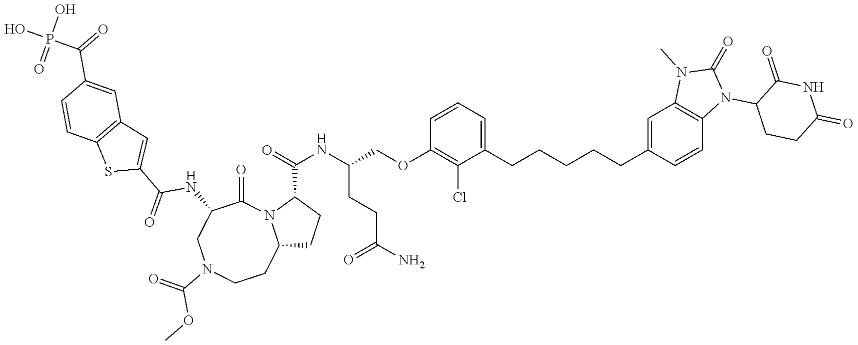 [SMILES: COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(=O)P(=O)(O)O)ccc3s2)C1] |
| I- 93 | 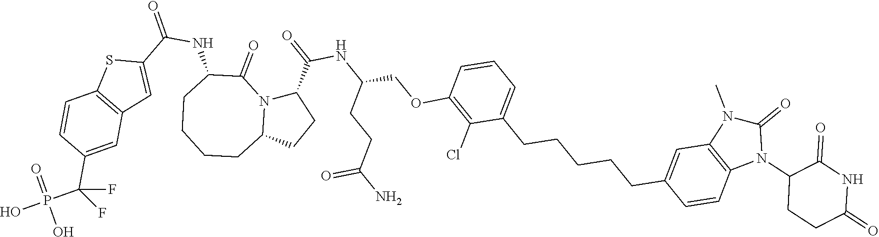 [SMILES: Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(F)(F)P(=O)(O)O)ccc7s6)C(=O)N54)c3Cl)cc21] |
| I- 94 | 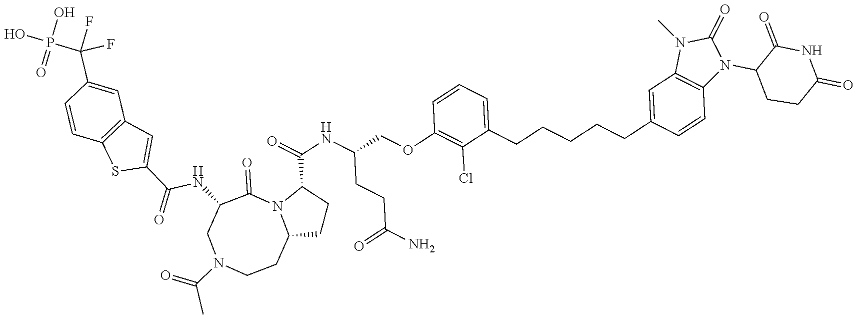 [SMILES: CC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(F)(F)P(=O)(O)O)ccc3s2)C1] |
| I- 95 | 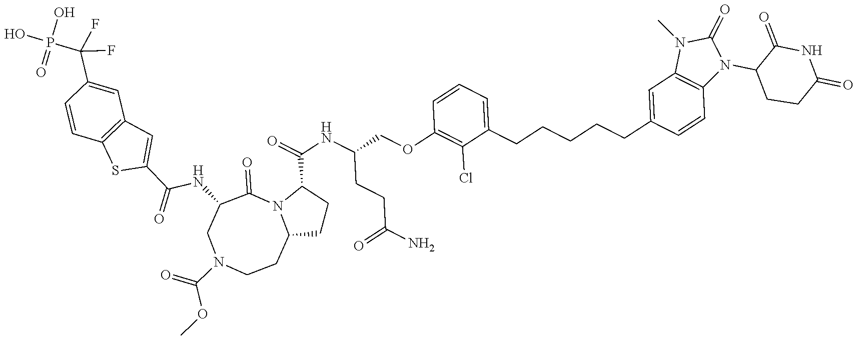 [SMILES: COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(F)(F)P(=O)(O)O)ccc3s2)C1] |
| I- 96 | 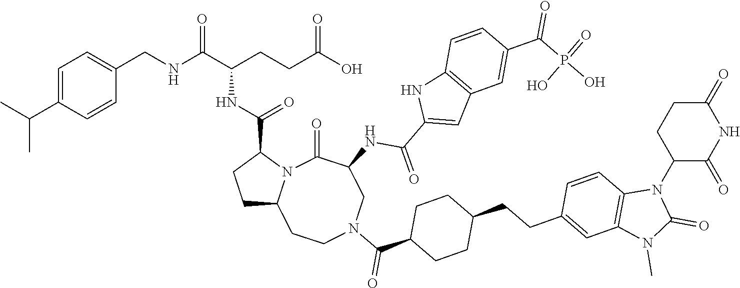 [SMILES: CC(C)c1ccc(CNC(=O)[C@H](CCC(=O)O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1] |
| I- 97 | 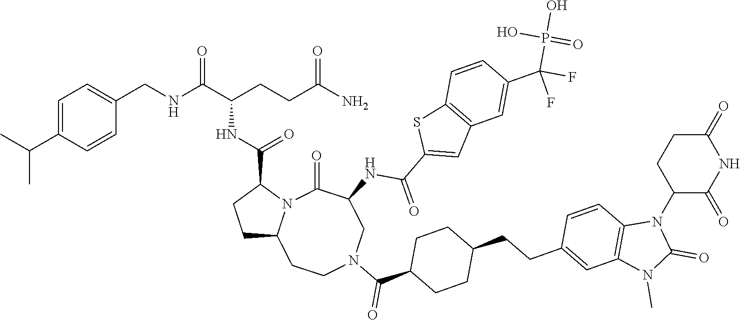 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(F)(F)P(=O)(O)O)ccc5s4)C(=O)N32)cc1] |
| I- 98 | 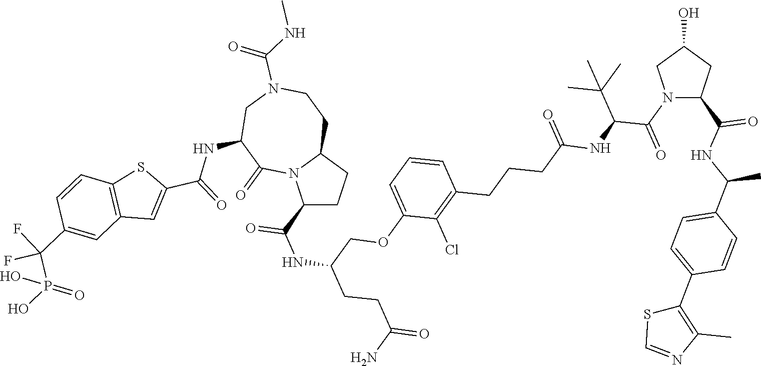 [SMILES: CNC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(F)(F)P(=O)(O)O)ccc3s2)C1] |

In some embodiments, the present invention provides a compound set forth in Table 1, above, or a pharmaceutically acceptable salt thereof. In some embodiments, the present invention provides a compound set forth in Table 1 as a diammonium salt.

In some embodiments, the present invention provides a compound of formula I, wherein the compound is not any of the compounds depicted in Table 1A, below.

TABLE 1A
Exemplary Compounds

| I-# | Structure |
|---|---|
| I-16 | 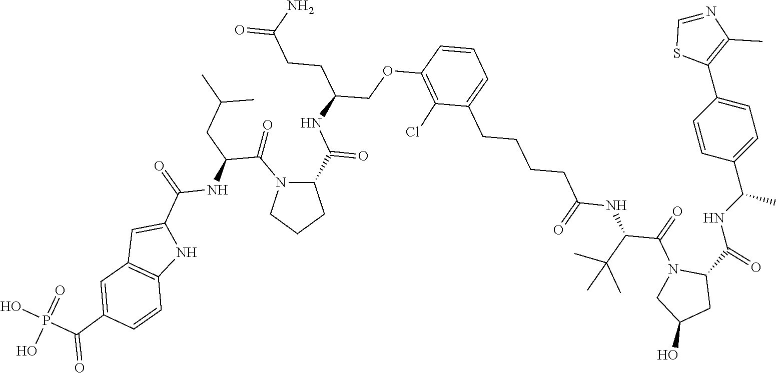 [SMILES: Cc1ncsc1-c1ccc([C@H](C)NC(=O)[C@@H]2C[C@@H](O)CN2C(=O)[C@@H](NC(=O)CCCCc2cccc(OC[C@H](CCC(N)=O)NC(=O)[C@@H]3CCCN3C(=O)[C@H](CC(C)C)NC(=O)c3cc4cc(C(=O)P(=O)(O)O)ccc4[nH]3)c2Cl)C(C)(C)C)cc1] |
| I-17 | 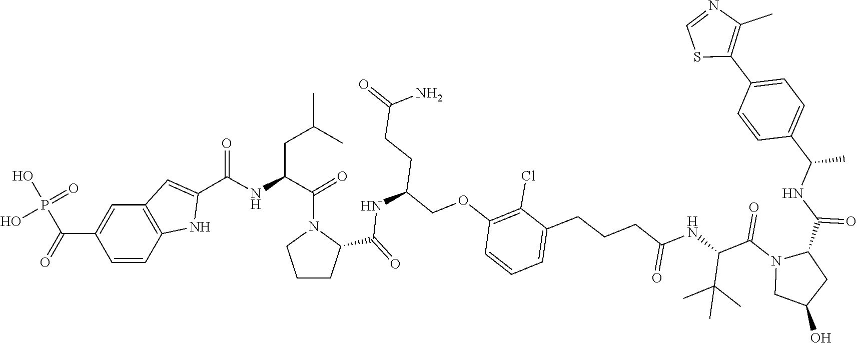 [SMILES: Cc1ncsc1-c1ccc([C@H](C)NC(=O)[C@@H]2C[C@@H](O)CN2C(=O)[C@@H](NC(=O)CCCc2cccc(OC[C@H](CCC(N)=O)NC(=O)[C@@H]3CCCN3C(=O)[C@H](CC(C)C)NC(=O)c3cc4cc(C(=O)P(=O)(O)O)ccc4[nH]3)c2Cl)C(C)(C)C)cc1] |
| I-18 | 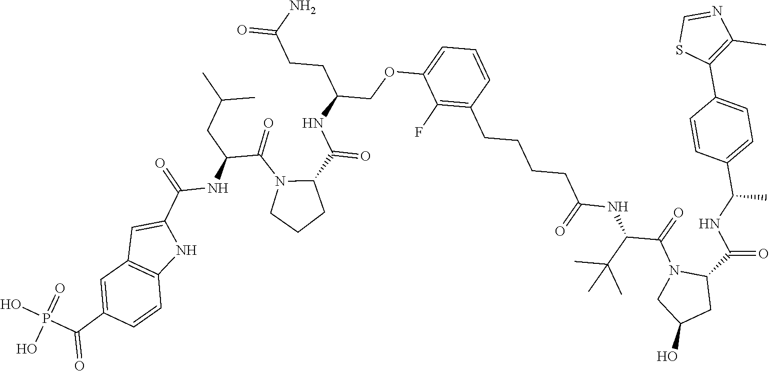 [SMILES: Cc1ncsc1-c1ccc([C@H](C)NC(=O)[C@@H]2C[C@@H](O)CN2C(=O)[C@@H](NC(=O)CCCCc2cccc(OC[C@H](CCC(N)=O)NC(=O)[C@@H]3CCCN3C(=O)[C@H](CC(C)C)NC(=O)c3cc4cc(C(=O)P(=O)(O)O)ccc4[nH]3)c2F)C(C)(C)C)cc1] |
| I-19 | 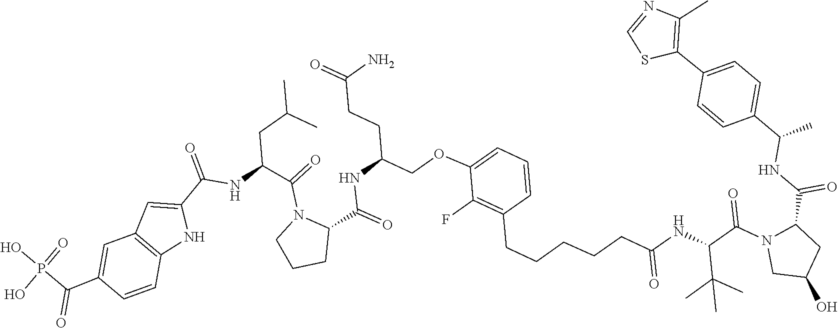 [SMILES: Cc1ncsc1-c1ccc([C@H](C)NC(=O)[C@@H]2C[C@@H](O)CN2C(=O)[C@@H](NC(=O)CCCCCc2cccc(OC[C@H](CCC(N)=O)NC(=O)[C@@H]3CCCN3C(=O)[C@H](CC(C)C)NC(=O)c3cc4cc(C(=O)P(=O)(O)O)ccc4[nH]3)c2F)C(C)(C)C)cc1] |

In some embodiments, the present invention provides a compound of formula I, wherein the compound is not any of the compounds depicted in Table 1A, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} a Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3rd edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a final degrader is formed having a free amine DIM moiety, it is not shown but it is generally appreciated and well known by those having ordinary skill in the art that the reactivity of said free amine may be masked by employing a suitable amino protecting group that can thereafter be removed in situ or during a separate synthetic step to form the final degrader product.

In certain embodiments, compounds of the present invention are generally prepared according to Scheme 1 set forth below:

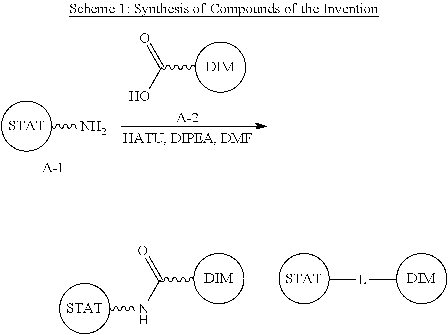

As depicted in Scheme 1, above, amine A-1 is coupled to acid A-2 using the coupling agent HATU in the presence of the base DIPEA in DMF to form a compound of the invention with a linker comprising an amide bond. The squiggly bond, [symbol] , represents the portion of the linker between STAT and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 2 set forth below:

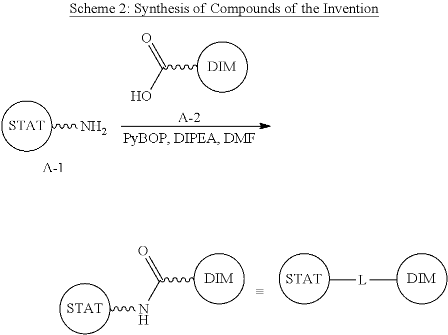

As depicted in Scheme 2, above, amine A-1 is coupled to acid A-2 using the coupling agent PyBOP in the presence of the base DIPEA in DMF to form a compound of the invention with a linker comprising an amide bond. The squiggly bond, [symbol] , represents the portion of the linker between STAT and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 3 set forth below:

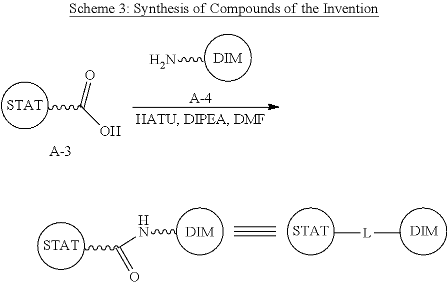

As depicted in Scheme 3, above, acid A-3 is coupled to amine A-4 using the coupling agent HATU in the presence of the base DIPEA in DMF to form a compound of the invention with a linker comprising an amide bond. The squiggly bond, [symbol] , represents the portion of the linker between STAT and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 4 set forth below:

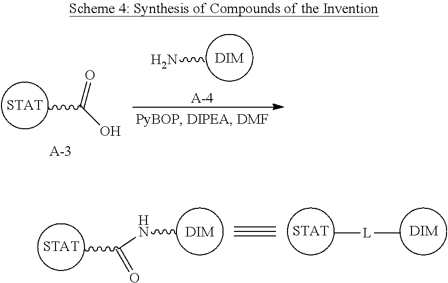

As depicted in Scheme 4, above, acid A-3 is coupled to amine A-4 using the coupling agent PyBOP in the presence of the base DIPEA in DMF to form a compound of the invention with a linker comprising an amide bond. The squiggly bond, [symbol] , represents the portion of the linker between STAT and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 5 set forth below:

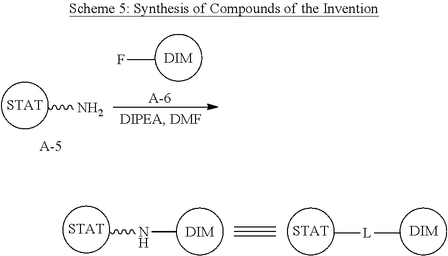

As depicted in Scheme 5, above, an S_{N}Ar displacement of fluoride A-6 by amine A-5 is effected in the presence of the base DIPEA in DMF to form a compound of the invention with a linker comprising a secondary amine. The squiggly bond, [symbol] , represents the portion of the linker between STAT and the terminal amino group of A-5.

In certain embodiments, compounds of the present invention are generally prepared according to Scheme 6 set forth below:

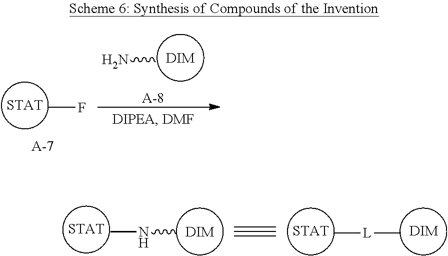

As depicted in Scheme 6, above, an S_{N}Ar displacement of fluoride A-7 by amine A-8 is effected in the presence of the base DIPEA in DMF to form a compound of the invention with a linker comprising a secondary amine. The squiggly bond, [symbol] , represents the portion of the linker between DIM and the terminal amino group of A-8.

In certain embodiments, compounds of the present invention are generally prepared according to Scheme 7 set forth below:

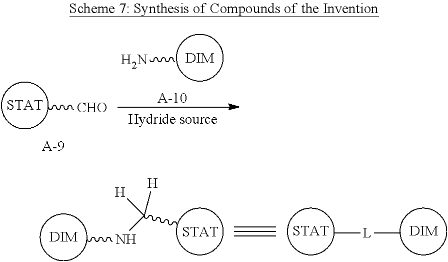

As depicted in Scheme 7, above, reductive alkylation of aldehyde A-9 by amine A-10 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between DIM and the terminal amino group of A-10.

In certain embodiments, compounds of the present invention are generally prepared according to Scheme 8 set forth below:

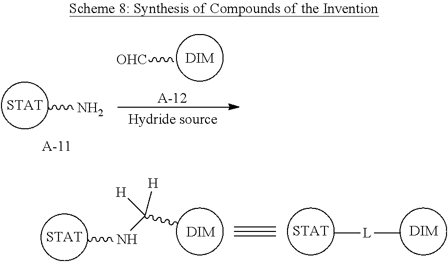

As depicted in Scheme 8, above, reductive alkylation of aldehyde A-12 by amine A-11 is effected in the presence of a mild hydride source (e.g., sodium cyanoborohydride or sodium triacetoxyborohydride) to form a provided compound with a linker comprising a secondary amine. The squiggly bond, [symbol] , represents' portion of the linker between STAT and the terminal amino group of A-11.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the invention are described below in the Exemplification.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a STAT protein, or a mutant thereof, in a biological sample or in a patient. In certain embodiments, the amount of compound in compositions of this invention is such that is effective to measurably degrade and/or inhibit an STAT protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a STAT protein, or a mutant thereof.

As used herein, the term “degratorily active metabolite or residue thereof” means that a metabolite or residue thereof is also a degrader of an STAT protein, or a mutant thereof.

In certain embodiments, a provided compound is administered as a prodrug.

The term “prodrug” refers to a compound that is made more active in vivo. A provided compound can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the provided compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as a phosphonate ester (the “prodrug”), but then is metabolically hydrolyzed to the phosphonic acid or a conjugate base thereof, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions ofthis invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the degradation and/or inhibition of STAT protein activity.

Examples of STAT protein that are degraded and/or inhibited by the compounds and compositions described herein and against which the methods described herein are useful include those of the signal transducer and activators of transcription (STAT) family of proteins, the members of which include STAT1, STAT2, STAT3, STAT4, STAT5A, STAT5B, or STAT6, or a mutant thereof. Yu et al., “Crosstalk between cancer and immune cells: Role of STAT3 in the tumour microenvironment” Nat. Rev. Immunol. 2007, 7, 41-51, Levy et al., “STATs: Transcriptional control and biological impact” Nat. Rev. Mol. Cell Biol. 2002, 3, 651-662, the entirety of each of which is herein incorporated by reference.

The activity of a compound utilized in this invention as a degrader and/or inhibitor of STAT1, STAT2, STAT3, STAT4, STAT5A, STAT5B, or STAT6, or a mutant thereof, may be assayed in vitro, in vivo or in a cell line. In vitro assays include assays that determine inhibition of either the activity and/or the subsequent functional consequences of activated STAT protein, or a mutant thereof. Alternate in vitro assays quantitate the ability of the inhibitor to bind to a STAT protein. Inhibitor binding may be measured by radiolabeling the inhibitor prior to binding, isolating the inhibitor/STAT complex and determining the amount of radiolabel bound. Alternatively, inhibitor binding may be determined by running a competition experiment where new inhibitors are incubated with a STAT protein bound to known radioligands. Representative in vitro and in vivo assays useful in assaying a STAT inhibitor include those described and disclosed in, e.g., Schust et al., “A high-throughput fluorescence polarization assay for signal transducer and activator of transcription 3” Anal. Biochem. 2004, 333(1):114; Müller et al., “A high-throughput assay for signal transducer and activator of transcription 5b based on fluorescence polarization” Anal. Biochem. 2008, 375(2):249. Detailed conditions for assaying a compound utilized in this invention as a degrader and/or inhibitor of STAT proteins, or a mutant thereof, are set forth in the Examples below.

The STAT family of proteins are cytoplasmic transcription factors with important roles in mediating responses to cytokines and growth factors, including promoting cell growth and differentiation, and inflammation and immune responses (Bromberg et al., Breast Cancer Res. 2000, 2:86-90; Darnell et al., Nat. Rev. Cancer 2002, 2:740-749). STAT proteins are classically activated by tyrosine (Tyr) kinases, such as Janus kinases (JAKs) and Src family kinases, in response to the binding of cytokine and growth factors to their cognate receptors (Darnell et al., Science 1994, 264:1415). The Tyr phosphorylation (pTyr) promotes dimerization between two activated STAT:STAT monomers through a reciprocal pTyr-Src homology SH2 domain interactions. Active STAT:STAT dimers translocate to the nucleus to induce gene transcription by binding to specific DNA-response elements in the promoters of target genes to regulate gene expression. By contrast, aberrantly-active STAT3, one of the STAT family members, has been implicated in many human tumors and represents an attractive target for drug discovery. Persistently activated STAT3 and, to some extent, STAT5 increase tumour cell proliferation, survival and invasion while suppressing anti-tumour immunity. The persistent activation of STAT3 also mediates tumour-promoting inflammation. This aberrant activation of STAT3 occurs in glioma, breast, prostate, ovarian, and many other human cancers, whereby it promotes malignant progression (Yu & Jove, Nat. Rev. Cancer 2004, 4:97-105). JAKs, Src, and epidermal growth factor receptor (EGFR) are STAT3 upstream regulators (Bromberg et al., Mol. Cell. Biol. 1998, 18:2553; Sartor et al., Cancer Res. 1997, 57:978; Garcia et al., Oncogene 2001, 20:2499). Mechanisms by which constitutively-active STAT3 mediates tumorigenesis include dysregulation of gene expression that leads to uncontrolled growth and survival of tumor cells, enhanced tumor angiogenesis, and metastasis and the suppression of tumor immune surveillance (Yu & Jove 2004; Bromberg & Darnell, Oncogene 2000, 19:2468-2473; Bowman et al., Oncogene 2000, 19:2474-2488; Turkson & Jove, Oncogene 2000, 19:6613-6626; Turkson, Expert Opin. Ther Targets 2004, 8:409-422; Wang et al., Nat. Med. 2004, 10:48-54).

The main domains of STAT3 protein include the tetramerization and leucine zipper at the N-terminus, the DNA binding domain, and the SH2 transactivation domain at the carboxy-terminal end. The SH2 region is responsible for the binding of STAT3 to the tyrosine-phosphorylated receptors and for the dimerization which is necessary for DNA binding and gene expression (Zhong et al., Science 1994, 264:95). STAT3 is activated by phosphorylation at Y-705, which leads to dimer formation, nuclear translocation, recognition of STAT3-specific DNA binding elements, and activation of target gene transcription (Darnell 1994; Zhong 1994).

The constitutive activation of STAT3 is frequently detected in breast carcinoma cell lines but not in normal breast epithelial cells (Garcia et al., Cell. Growth. Differ. 1997, 8:1267; Bowman 2000). It has been reported that approximately 60 percent of breast tumors contain persistently activated STAT3 (Dechow et al., Proc. Natl. Acad. Sci. USA 2004, 101:10602). STAT3 has been classified as a proto-oncogene because activated STAT3 can mediate oncogenic transformation in cultured cells and tumor formation in nude mice (Bromberg et al., Cell 1999, 98:295). STAT3 may participate in oncogenesis by stimulating cell proliferation, promoting angiogenesis, and conferring resistance to apoptosis induced by conventional therapies (Catlett-Falcone et al., Curr. Opin. Oncol. 1999, 11:1; Catlett-Falcone et al., Immunity 1999, 10:105; Alas et al., Clin. Cancer Res. 2003, 9:316; Wei et al., Oncogene 2003, 22:1517). Possible downstream targets through which STAT3 promotes oncogenesis include up-regulation of anti-apoptotic factors (Bcl-2, survivin, Mcl-1, and Bcl-X_{L}), cell-cycle regulators (cyclin D1, MEK5, and c-myc), and inducer of tumor angiogenesis (VEGF) (Bromberg et al., Cell 1999, 98:295; Wei et al., Oncogene 2003, 22:1517; Real et al., Oncogene 2002, 21:7611; Puthier et al., Eur. J. Immunol. 1999, 29:3945; Niu et al., Oncogene 2002, 21:2000; Kiuchi et al., J. Exp. Med. 1999, 189:63; Song et al., Oncogene 2004, 23:8301). Activated STAT3 signaling directly contributes to malignant progression of cancer. STAT3 oncogenic function acts through the pro-survival proteins such as survivin, Mcl-1, Bcl-2, and Bcl-X_{L} and results in the prevention of apoptosis (Real et al., Oncogene 2002, 21:7611; Aoki et al., Blood 2003, 101:1535; Epling-Burnette et al., J. Clin. Invest. 2001, 107:351; Nielsen et al., Leukemia 1999, 13:735). Blockade of STAT3 signaling inhibits cancer cell growth, demonstrating that STAT3 is essential to the survival or growth of tumor cells (Alas et al., Clin. Cancer Res. 2003, 9:316; Aoki et al., Blood 2003, 101:1535; Epling-Bumette et al., J Clin. Invest. 2001, 107:351; Burke et al., Oncogene 2001, 20:7925; Mora et al., Cancer Res. 2002, 62:6659; Ni et al., Cancer Res. 2000, 60:1225; Rahaman et al., Oncogene 2002, 21:8404).

Recent evidence also reveals the role of STAT3 in modulating mitochondrial functions and STAT3 crosstalk with other proteins, such as NF-κB, that promotes the malignant phenotype. Many human tumors harbor aberrantly-active STAT3 signaling, and studies in experimental models indicate tumor cells and tumors harboring constitutively-active STAT3 are responsive to STAT3 signaling modulators (Gough et al., Science 2009, 324:1713; Yu et al., Nat. Rev. Cancer 2009, 9:798; Grivennikov & Karin, Cytokine & Growth Factor Rev. 2010, 21:11).

Representative STAT inhibitors include those described and disclosed in e.g., Morlacchi et al. Future Med. Chem. 2014, 6(7):1909; Sgrignani et al. Int. J. Mol. Sci. 2018, 19:1591, Botta et al. Mol. Inf 2015, 34:689; Leung et al. Methods 2015, 71:38; Lavecchia et al. Cur Med. Chem. 2011, 18:1; Chun et al. Can. Lett. 2015, 357:393; Zhang et al. Eur J Med. Chem. 2017, 125:538; Yesylevskyy et al. J. Chem. Inf Model. 2016, 56:1588; Huang et al. Bioorg. Med. Chem. Lett. 2016, 26:5172; Gao et al. Bioorg. Med. Chem. 2016, 24:2549; Daka et al. Bioorg. Med. Chem. 2015, 23:1348; Ji et al. Bioorg. Med. Chem. 2016, 24:6174; Zhou et al. Bioorg. Med. Chem. 2017, 25:2995; and Yu et al. J. Med. Chem. 2017, 60:2718; Chen et al. Med. Chem. Lett. 2010, 1:85; the entirety of each of which is herein incorporated by reference.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of one of more STAT protein and are therefore useful for treating one or more disorders associated with activity of one or more of STAT protein. Thus, in certain embodiments, the present invention provides a method for treating a STAT1-mediated, STAT2-mediated, STAT3-mediated, STAT4-mediated, STAT5A-mediated, STAT5B-mediated, or STAT6-mediated disorder comprising the step of administering to a patient in need thereof a compound of the present invention, or pharmaceutically acceptable composition thereof.

As used herein, the terms “STAT1-mediated”, “STAT2-mediated”, “STAT3-mediated”, “STAT4-mediated”, “STAT5A-mediated”, “STAT5B-mediated”, and/or “STAT6-mediated” disorders, diseases, and/or conditions as used herein means any disease or other deleterious condition in which one or more STAT1, STAT2, STAT3, STAT4, STAT5A, STAT5B, or STAT6, or a mutant thereof, are known to play a role. Accordingly, another embodiment of the present invention relates to treating or lessening the severity of one or more diseases in which one or more STAT1, STAT2, STAT3, STAT4, STAT5A, STAT5B, or STAT6, or a mutant thereof, are known to play a role.

In some embodiments, the present invention provides a method for treating one or more disorders, diseases, and/or conditions wherein the disorder, disease, or condition is a cancer, a neurodegenative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer (see, e.g., Turkson & Jove, Oncogene 2000, 19:6613-6626), diabetes (see, e.g., Gurzov et al., FEBS 2016, 283:3002), cardiovascular disease (see, e.g., Grote et al., Vasc. Pharmacol. 2005, 43:2005), viral disease (see, e.g., Gao et al., J. Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9):1650), autoinflammatory syndromes (see, e.g., Rauch et al., Jak-Stat 2013, 2(1): e23820), atherosclerosis (see, e.g., Ortiz-Munoz et al., Arterio., Thrombo., Vasc. Bio. 2009, 29:525), psoriasis (see, e.g., Andrés et al., Exp. Derm. 2013, 22(5):323), allergic disorders (see, e.g., Oh et al., Eur. Respir. Rev. 2019, 19(115):46), inflammatory bowel disease (see, e.g., Sugimoto, World J. Gastroenterol. 2008, 14(33):5110), inflammation (see, e.g., Tamiya et al., Arterio., Thrombo., Vasc. Bio. 2011, 31:980), acute and chronic gout and gouty arthritis, neurological disorders (see, e.g., Campbell, Brain Res. Rev. 2005, 48(2):166), metabolic syndrome, immunodeficiency disorders such as AIDS and HIV (see, e.g., O'Shea et al., N. Engl. J Med. 2013, 368:161), destructive bone disorders (see, e.g., Jatiani et al., Genes & Can. 2011, 1(10):979), osteoarthritis, proliferative disorders, Waldenström's Macroglobulinemia (see, e.g., Hodge et al., Blood 2014, 123(7):1055) infectious diseases, conditions associated with cell death, pathologic immune conditions involving T cell activation, and CNS disorders in a patient. In one embodiment, a human patient is treated with a compound of the current invention and a pharmaceutically acceptable carrier, adjuvant, or vehicle, wherein said compound is present in an amount to measurably degrade and/or inhibit one or more STAT1, STAT2, STAT3, STAT4, STAT5A, STAT5B, or STAT6, or a mutant thereof

Compounds of the current invention are useful in the treatment of a proliferative disease selected from a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenström's macroglobulinemia (WM), splenic marginal zone lymphoma, multiple myeloma, plasmacytoma, intravascular large B-cell lymphoma).

In some embodiments, the aberrant activation of STAT3 which can be treated according to the methods of this invention is a human cancer. In some embodiments, the human cancer which can be treated according to the methods of this invention is selected from glioma, breast cancer, prostate cancer, head and neck squamous cell carcinoma, skin melanomas, ovarian cancer, malignant peripheral nerve sheath tumors (MPNST), and pancreatic cancer. In some embodiments, abnormal STAT3 activation also correlates with the progression of diverse hematopoietic malignancies, such as various leukemias and lymphomas, and STAT3 is frequently activated in both multiple myeloma cell lines and tumor cell lines derived from patient bone marrows.

In some embodiments, the present invention provides a method of treating a cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes.

In some embodiments, the present invention provides a method of treating a JAK-associated disease. In some embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

In some embodiments, the present invention provides a method oftreating triple negative breast cancer in a patient in need thereof, comprising administering a compound of the present invention, or a pharmaceutically acceptable salt thereof.

In some embodiments, the present invention provides a method of treating malignant peripheral nerve sheath tumors (MPNST) in a patient in need thereof, comprising administering a compound of the present invention, or a pharmaceutically acceptable salt thereof.

In some embodiments, the present invention provides a method of treating lung cancer in a patient in need thereof, comprising administering a compound of the present invention, or a pharmaceutically acceptable salt thereof.

In some embodiments, the present invention provides a method of treating NSCLC in a patient in need thereof, comprising administering a compound of the present invention, or a pharmaceutically acceptable salt thereof.

In some embodiments, the present invention provides a method of treating EGFR-mutant NSCLC in a patient in need thereof, comprising administering a compound of the present invention, or a pharmaceutically acceptable salt thereof.

In some embodiments, the present invention provides a method of treating colorectal cancer in a patient in need thereof, comprising administering a compound of the present invention, or a pharmaceutically acceptable salt thereof.

In some embodiments, the present invention provides a method of treating peripheral T-cell lymphoma in a patient in need thereof, comprising administering a compound of the present invention, or a pharmaceutically acceptable salt thereof.

In some embodiments, the present invention provides a method of treating pancreatic cancer in a patient in need thereof, comprising administering a compound of the present invention, or a pharmaceutically acceptable salt thereof.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds according to the invention are useful in the treatment of heteroimmune diseases. Examples of such heteroimmune diseases include, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, the present invention provides a method of treating an autoimmune disease selected from encephalomyelitis, systemic sclerosis, idiopathic pulmonary fibrosis, inflammatory bowel disease, atopic dermatitis, rheumatoid arthritis, graft versus host disease (acute and chronic), and other tissue fibrosis diseases.

In some embodiments, the present invention provides a method of treating autoimmune encephalomyelitis in a patient in need thereof, comprising administering a compound of the present invention, or a pharmaceutically acceptable salt thereof.

In some embodiments, the present invention provides a method of treating a hematologic malignancy selected from LGL leukemia (T and NK cell), cutaneous T cell lymphoma (CTCL), peripheral T cell lymphomas (PTCL, all subtypes including ALCL), diffuse large B cell lymphoma (DLBCL), acute myelogenous leukemia, multiple myeloma, and myelofibrosis

In some embodiments, the present invention provides a method of treating tissue fibrosis or chronic tissue disease, including liver and kidney fibrosis, in a patient in need thereof, comprising administering a compound of the present invention, or a pharmaceutically acceptable salt thereof.

In some embodiments, the present invention provides a method of treating idiopathic interstitial pneumonia(s) (IIPs), including any type of lung fibrosis, either interstitial lung disease associated with rheumatoid disease (including SSc) or IPF itself, in a patient in need thereof, comprising administering a compound of the present invention, or a pharmaceutically acceptable salt thereof.

In some embodiments the inflammatory disease which can be treated according to the methods of this invention is an disease of the skin. In some embodiments, the inflammatory disease of the skin is selected from contact dermatitis, atopic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods of this invention is a TH17 mediated disease. In some embodiments the TH17 mediated disease is selected from Systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Cardiovascular diseases which can be treated according to the methods of this invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of this invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV infection.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,
In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

In some embodiments, the present invention provides a method of treating EGFR-mutant NSCLC in a patient in need thereof, comprising administering a compound of the present invention or a pharmaceutically acceptable salt thereof and one or more EGFR kinase inhibitors (e.g., gefitinib, erlotinib, lapatinib, afatinib, osimertinib, brigatinib, etc.).

In some embodiments, the present invention provides a method of treating EGFR-mutant NSCLC in a patient in need thereof, comprising administering a compound of the present invention or a pharmaceutically acceptable salt thereof and erlotinib.

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity or degading a protein kinase in a biological sample comprising the step of contacting said biological sample with a compound of this invention, or a composition comprising said compound.

According to another embodiment, the invention relates to a method of inhibiting or degrading STAT1, STAT2, STAT3, STAT4, STAT5A, STAT5B, or STAT6, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition and/or degradation of a STAT protein, or a protein selected from STAT1, STAT2, STAT3, STAT4, STAT5A, STAT5B, or STAT6, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting one or more of STAT1, STAT2, STAT3, STAT4, STAT5A, STAT5B, or STAT6, or a mutant thereof, activity in a patient comprising the step of administering to said patient a compound of the present invention, or a composition comprising said compound. In other embodiments, the present invention provides a method for treating a disorder mediated by one or more of STAT1, STAT2, STAT3, STAT4, STAT5A, STAT5B, or STAT6, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-γ, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid^{T}M) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R¹¹⁵⁷⁷⁷ (Zamestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™),) carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment ofAML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTOR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-0, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIRI, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/69264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-lh68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15R^{A}), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those described in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, STAT3 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-mnethvmnorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

Analytical instruments Table:

| | |
|---|---|
| LCMS | Shimadzu UFLC MS: LCMS-2020 |
| | Agilent Technologies 1200 series MS: Agilent |
| | Technologies 6110 Agilent Technologies 1200 |
| | series MS: LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×⁵⁰ mm columns packed with 5 mm C18-coated silica or Kinetex EVO C18 2.1×³⁰ mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

### Intermediates

### (3S,6S,10aS)-6-[(tert-butoxycarbonyl)amino]-5-oxo-octahydro-1H-pyrrolo[1,2-a]azocine-3-carboxylic acid (Intermediate A)

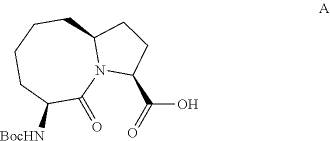 [SMILES: CC(C)(C)OC(=O)N[C@H]1CCCC[C@H]2CC[C@@H](C(=O)O)N2C1=O]

(3S,6S,10aS)-6-[(tert-butoxycarbonyl)amino]-5-oxo-octahydro-1H-pyrrolo[1,2-a]azocine-3-carboxylic acid was synthesized as described in Sun et al., J. Am. Chem. Soc. 2007, 129(49):15279-15294 and WO 2007130626.

### 2-(2,3,4,5,6-Pentafluorophenoxycarbonyl)-1H-indole-5-carbonylphosphonic acid (Intermediate B)

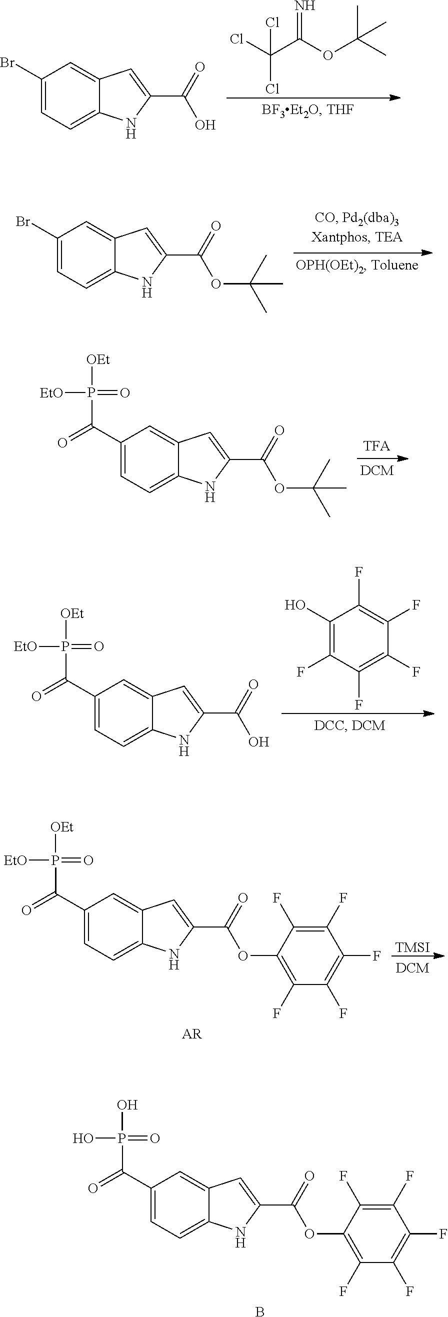

Step 1—Tert-butyl 5-bromo-1H-indole-2-carboxylate. To a stirred solution of 5-bromo-1H-indole-2 carboxylic acid (20.00 g, 83.31 mmol, CAS #7254-19-5) in THF (250.00 mL) was added tert-butyl 2,2,2-trichloroethanimidate (45.51 g, 208.29 mmol) in portions at 25° C. under nitrogen atmosphere. To the above mixture was added BF₃Et₂O (2.36 g, 16.66 mmol) dropwise over 10 min at 0° C. The resulting mixture was stirred for overnight at rt. On completion, the reaction was quenched with saturated aq. Na₂CO₃ (200 mL) and diluted with water (100 mL). The mixture was then extracted with EtOAc (3×100 mL), the combined organic layers were washed with water (3×100 mL), and dried over anhydrous Na₂SO₄. After filtration, the filtrate was concentrated under reduced pressure and the residue was purified by silica gel column chromatography, eluted with PE/EtOAc (20/1), to afford the title compound (22.8 g, 92% yield) as a light yellow solid; ¹H NMR (400 MHz, DMSO-d₆) δ 11.90 (s, 1H), 7.86 (d, J=1.9 Hz, 1H), 7.42 (d, J=8.8 Hz, 1H), 7.36 (dd, J=8.8, 1.9 Hz, 1H), 7.03 (dd, J=2.2, 0.9 Hz, 1H), 1.57 (s, 9H); LC/MS (ESI, m z): [(M−1)]⁻=293.9, 295.9.

Step 2—Tert-butyl 5-((diethoxyphosphoryl)carbonyl)-1H-indole-2-carboxylate. To a stirred mixture of tert-butyl 5-bromo-1H-indole-2-carboxylate (20.00 g, 67.53 mmol) in toluene (300.00 mL) were added Pd₂(dba)₃-CHCl₃ (3.5 g, 3.4 mmol), XantPhos (1.96 g, 3.38 mmol) and TEA (6.84 g, 67.53 mmol) in turns at rt. The reaction system was degassed under vacuum and purged with CO several times and stirred under CO balloon (˜1 atm) at 25° C. for 10 min. Then diethylphosphonate (9.32 g, 67.53 mmol) was added to above mixture and the resulting mixture was stirred for 4 h at 90° C. under CO atmosphere. On completion, the reaction mixture was filtered and the filter cake was washed with DCM (3×15 mL). The filtrate was concentrated under reduced pressure and the crude product was purified by reverse phase flash with the following conditions (Column: Spherical C18, 20-40 um, 330 g; Mobile Phase A: Water (0.05% FA), Mobile Phase B: ACN; Flow rate: 80 mL/min; Gradient (B %): 5%˜5%, 8 min; 40%˜70%, 30 min; 95%, 5 min; Detector: 254 nm; Rt: 35 min.) to afford the title compound (18 g, 70% yield) as a yellow solid. ¹H NMR (400 MHz, DMSO-d₆) δ 12.28 (s, 1H), 8.76 (d, J=1.7 Hz, 1H), 7.97 (dd, J=8.9, 1.7 Hz, 1H), 7.63-7.57 (m, 1H), 7.37-7.34 (m, 1H), 4.23-4.15 (m, 4H), 1.58 (s, 9H), 1.29 (t, J=7.0 Hz, 6H); LC/MS (ESI, m/z): [(M+1)]⁺=382.1.

Step 3—5-((Diethoxyphosphoryl)carbonyl)-1H-indole-2-carboxylic acid. To a stirred solution of tert-butyl 5-[(diethoxyphosphoryl)carbonyl]-1H-indole-2-carboxylate (55.00 g, 144.22 mmol) in DCM (1100 mL) was added TFA (500 mL) dropwise at rt and the resulting mixture was stirred for 2 h at rt under nitrogen atmosphere. On completion, the resulting mixture was concentrated under reduced pressure. The residue was triturated with Et₂O (1000 mL) to afford the title compound (46 g, 98% yield) as a light brown solid: ¹H NMR (400 MHz, DMSO-d₆) δ 12.36 (s, 1H), 8.78 (d, J=1.7 Hz, 1H), 7.96 (dd, J=8.9, 1.7 Hz, 1H), 7.61-7.55 (m, 1H), 7.39 (dd, J=2.1, 0.9 Hz, 1H), 4.21-4.18 (m, 4H), 1.29 (t, J=7.0 Hz, 6H); LC/MS (ESI, m z): [(M+1)]⁺=326.1.

Step 4—2,3,4,5,6-pentafluorophenyl 5-[(diethoxyphosphoryl)carbonyl]-1H-indole-2-carboxylate (Intermediate AR). To a stirred solution of 5-[(diethoxyphosphoryl)carbonyl]-1H-indole-2-carboxylic acid (100.00 g, 307.45 mmol) and pentafluorophenol (84.89 g, 461.17 mmol) in DCM (1.50 L) was added DCC (95.15 g, 461.17 mmol) at rt under air atmosphere. The resulting mixture was stirred overnight at rt under nitrogen atmosphere. On completion, the resulting mixture was filtered and the filter cake was washed with DCM (3×100 mL). The filtrate was then concentrated under reduced pressure to 300 mL of DCM and the mixture was diluted with hexane (1 L). The precipitated solids were collected by filtration and washed with hexane (3×100 mL) to afford the title compound (150 g, contains trace amount of DCU) as a light yellow solid.

Step 5—2-(2,3,4,5,6-pentafluorophenoxycarbonyl)-1H-indole-5-carbonylphosphonic acid. To a stirred solution of 2,3,4,5,6-pentafluorophenyl 5-[(diethoxyphosphoryl)carbonyl]-1H-indole-2-carboxylate (65.00 g, 132.30 mmol) in anhydrous DCM (1300 mL) was added TMSI (79.42 g, 396.90 mmol) dropwise at rt under Argon atmosphere. The resulting mixture was stirred for 30 min at rt under Argon atmosphere. On completion, the mixture was concentrated under reduced pressure. The residue was then dissolved in dry MeCN (500 mL), then sat. aq. Na₂S₂O₃ (50 mL) was dropwise into the solution until the dark brown solution changed to light yellow and a pecipitate was generated. The suspension was filtered and the filter cake was washed with ACN/water (10/1, 50 mL, three times) and collected. The collected solid was triturated with Et2O (500 ml) to afford the title compound (45 g, 78%) as an off-white solid. LCMS (ESI, m/z): (M+1)⁺434.0.

### 5-[3-[(2S)-2-[(tert-Butoxycarbonyl)amino]-4-carbamoylbutoxy]-2-chlorophenyl]pentanoic acid (Intermediate C)

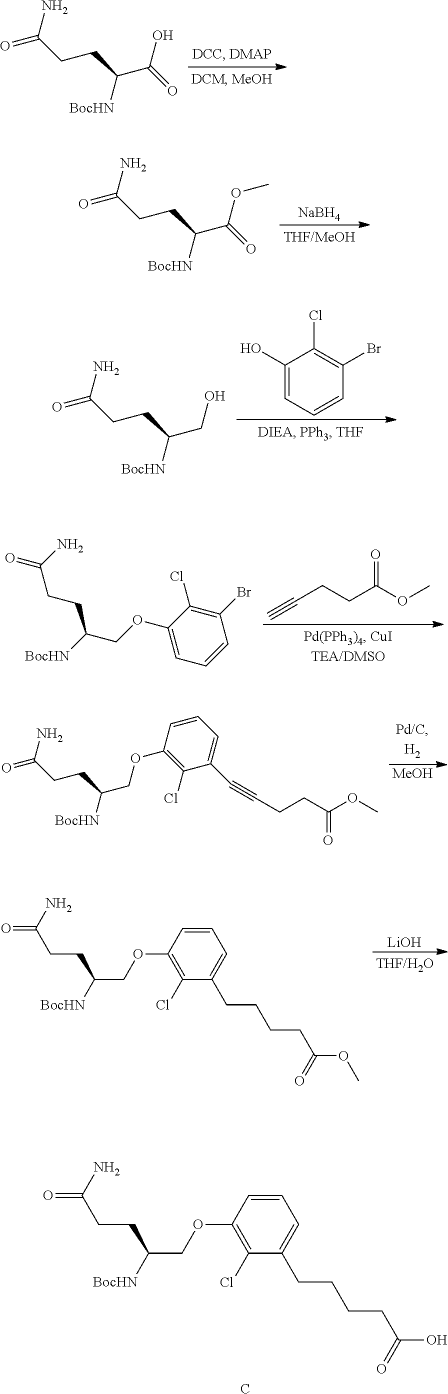

Step 1—Methyl (2S)-2-[(tert-butoxycarbonyl)amino]-4-carbamoyl butanoate. To a solution of (2S)-2-[(tert-butoxycarbonyl)amino]-4-carbamoylbutanoic acid (200 g, 812 mmol, CAS #13726-85-7) in DCM (2 L) were added MeOH (130 g, 4.06 mol), DCC (201 g, 0.97 mol) and DMAP (9.92 g, 81 mmol) in portions at rt under nitrogen atmosphere. The resulting mixture was stirred for 16 h at rt under nitrogen atmosphere. On completion, the reaction mixture was filtered and the filtered cake was washed with DCM (300 mL×3). The filtrates were concentrated under reduced pressure and the residue was purified by silica gel column chromatography, eluted with 1% methanol MeOH in ethyl acetate, to afford the title compound (180 g, 85% yield) as a white solid: ¹H NMR (400 MHz, DMSO-d₆) δ 7.29-7.22 (m, 2H), 6.77 (s, 1H), 3.95 (td, J=8.6, 5.0 Hz, 1H), 3.62 (s, 3H), 2.13 (t, J=7.5 Hz, 2H), 1.91 (dd, J=13.3, 5.9 Hz, 1H), 1.72 (dd, J=14.2, 8.1 Hz, 1H), 1.38 (s, 9H); LC/MS (ESI, m z): [(M+1)]⁺=261.2.

Step 2—Tert-butyl N-[(25)-4-carbamoyl-1-hydroxybutan-2-yl]carbamate. To a solution of methyl (2S)-2-[(tert-butoxycarbonyl)amino]-4-carbamoylbutanoate (180 g, 0.69 mol) in MeOH (800 mL) and THF (1.6 L) was added NaBH₄(52 g, 1.38 mol) in portions at 0° C. under nitrogen atmosphere. The resulting mixture was stirred for 16 h at rt. On completion, the mixture was concentrated under reduced pressure. The residue was purified by silica gel column chromatography, eluted with 10% methanol in dichloromethane, to afford the title compound (150 g, 93% yield) as a white solid: ¹H NMR (400 MHz, DMSO-d₆) δ 7.23 (s, 1H), 6.69 (s, 1H), 6.47 (d, J=8.1 Hz, 1H), 4.58 (br, 1H), 3.33 (p, J=5.4 Hz, 2H), 3.26-3.17 (m, 1H), 2.04 (q, J=7.7 Hz, 2H), 1.80-1.66 (m, 1H), 1.47-1.43 (m, 1H), 1.38 (s, 9H); LC/MS (ESI, m z): [(M+1)]⁺=233.2.

Step 3—Tert-butyl N-[(25)-4-carbamoyl-1-hydroxybutan-2-yl]carbamate. To a solution of 3-bromo-2-chlorophenol (21.4 g, 103 mmol) in THF (200 mL) were added PPh₃ (27.1 g, 103 mmol) and DEAD (18 g, 103 mmol) in portions at 0° C. under nitrogen atmosphere. Then a solution of tert-butyl N-[(2S)-4-carbamoyl-1-hydroxybutan-2-yl]carbamate (20 g, 86 mmol) in DMF (30 mL) was added to the above solution over 10 min. The resulting mixture was stirred for 3 h at rt under nitrogen atmosphere. On completion, the reaction mixture was concentrated under reduced pressure. The residue was purified by silica gel column chromatography, eluted with 17% ethyl acetate in petroleum ether, to afford the title compound (13 g, 36% yield) as a white solid: ¹H NMR (300 MHz, DMSO-d₆) δ 7.66-7.57 (m, 1H), 7.32 (dd, J=7.8, 1.7 Hz, 1H), 7.23 (dd, J=10.4, 5.6 Hz, 2H), 6.80 (d, J=8.4 Hz, 1H), 6.72 (s, 1H), 4.06-3.89 (m, 2H), 3.78-3.74 (m, 1H), 2.24-2.10 (m, 1H), 2.15-2.01 (m, 1H), 1.91-1.73 (m, 1H), 1.63-1.59 (m, 1H), 1.37 (s, 9H); LC/MS (ESI, m z): [(M+1)]⁺=421.0, 423.0.

Step 4—Methyl 5-[3-[(2S)-2-[(tert-butoxycarbonyl)amino]-4-carbamoylbutoxy]-2-chlorophenyl]pent-4-ynoate. To a stirred solution of tert-butyl N-[(2S)-1-(3-bromo-2-chlorophenoxy)-4-carbamoylbutan-2-yl]carbamate (4 g, 10 mmol) in DMSO (30 mL) were added methyl pent-4-ynoate (3.19 g, 28.4 mmol, CAS #21565-82-2), TEA (10 mL), Pd(PPh₃)₄ (1.10 g, 0.95 mmol) and CuI (180 mg, 0.95 mmol) at rt under nitrogen atmosphere. The resulting mixture was then stirred for 3 h at 80° C. under nitrogen atmosphere. On completion, the mixture was cooled to rt and filtered. The filtered cake was washed with DCM (3×100 mL) and the filtrates were concentrated under reduced pressure. The residue was purified by reverse flash chromatography (Column: Spherical C18, 20˜40 um, 330 g; Mobile Phase A: Water (plus 10 mM NH₄CO₃); Mobile Phase B: ACN; Flow rate: 85 mL/min; Gradient of B: 5%, 12 min; 5%˜35%, 10 min; 35%˜75%, 15 min; 75%˜95%, 10 min, Detector: UV 254/220 nm, desired product were collected at 55% B) and concentrated under reduced pressure and lyophilized to afford the title compound (4.2 g, 98% yield) as a white solid: ¹H NMR (300 MHz, DMSO-d₆) δ 7.31-7.20 (m, 2H), 7.15 (d, J=8.2 Hz, 1H), 7.11-7.03 (m, 1H), 6.83 (d, J=8.4 Hz, 1H), 6.75 (s, 1H), 3.96 (dd, J=6.2, 3.0 Hz, 2H), 3.78 (s, 1H), 3.65 (s, 3H), 2.80-2.59 (m, 4H), 2.16-2.12 (m, 2H), 1.85-1.81 (s, 1H), 1.63-1.59 (m, 1H), 1.40 (s, 9H); LC/MS (ESI, m z): [(M+1)]⁺=453.1.

Step 5—Methyl 5-[3-[(2S)-2-[(tert-butoxycarbonyl)amino]-4-carbamoylbutoxy]-2-chlorophenyl]pentanoate. To a stirred solution of methyl 5-[3-[(2S)-2-[(tert-butoxycarbonyl)amino]-4-carbamoylbutoxy]-2-chlorophenyl]pent-4-ynoate (4.00 g, 8.83 mmol) in MeOH (40 mL) was added PtO₂ (200 mg, 0.88 mmol) in portions at rt under nitrogen atmosphere. The resulting mixture was stirred for 16 h at rt under hydrogen atmosphere (1.5 atm). On completion, the reaction mixture was filtered and the filtered cake was washed with MeOH (3×10 mL). The filtrates were concentrated under reduced pressure to afford the title compound (3.7 g, 92% yield) as a white solid: ¹H NMR (400 MHz, DMSO-d₆) δ 7.27 (s, 1H), 7.20 (t, J=7.9 Hz, 1H), 6.98 (dd, J=8.4, 1.4 Hz, 1H), 6.90 (dd, J=7.7, 1.3 Hz, 1H), 6.81 (d, J=8.5 Hz, 1H), 6.77-6.72 (m, 1H), 3.92 (d, J=6.1 Hz, 2H), 3.82-3.72 (m, 1H), 3.58 (s, 3H), 2.69 (s, 2H), 2.38-2.29 (m, 2H), 2.23-2.05 (m, 2H), 1.91-1.78 (m, 1H), 1.69-1.59 (m, 1H), 1.59-1.51 (m, 4H), 1.39 (s, 9H); LC/MS (ESI, m z): [(M+1)]⁺=457.2.

Step 6—5-[3-[(2S)-2-[(tert-butoxycarbonyl)amino]-4-carbamoylbutoxy]-2-chlorophenyl]pentanoic acid. To a stirred solution of methyl 5-[3-[(2S)-2-[(tert-butoxycarbonyl)amino]-4-carbamoylbutoxy]-2-chlorophenyl]pentanoate (3.70 g, 8.10 mmol) in THF (40 mL) was added LiOH (1.94 g, 80.97 mmol) in H₂O (40 mL) dropwise at rt under air atmosphere. The resulting mixture was stirred for 2 h at rt under air atmosphere. On completion, the mixture was acidified to pH=4 with conc. HCl. The mixture was then extracted with CH₂Cl₂ (3×60 mL). The combined organic layers were washed with brine (2×60 mL) and dried over anhydrous Na₂SO₄. After filtration, the filtrate was concentrated under reduced pressure to afford the title compound (3.3 g, 92% yield) as a white solid: ¹H NMR (400 MHz, DMSO-d₆) δ 12.00 (s, 1H), 7.30-7.25 (m, 1H), 7.20 (t, J=7.9 Hz, 1H), 6.98 (dd, J=8.3, 1.4 Hz, 1H), 6.91 (dd, J=7.6, 1.3 Hz, 1H), 6.81 (d, J=8.4 Hz, 1H), 6.75 (s, 1H), 3.92 (d, J=6.1 Hz, 2H), 3.82-3.72 (m, 1H), 2.69 (t, J=6.9 Hz, 2H), 2.28-2.21 (m, 2H), 2.19-2.05 (m, 2H), 1.90-1.72 (m, 2H), 1.68-1.49 (m, 4H), 1.39 (s, 9H); LC/MS (ESI, m z): [(M+1)]⁺=443.1.

### (2S,4R)-1-[(2S)-2-(5-[3-[(2S)-2-Amino-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate D)

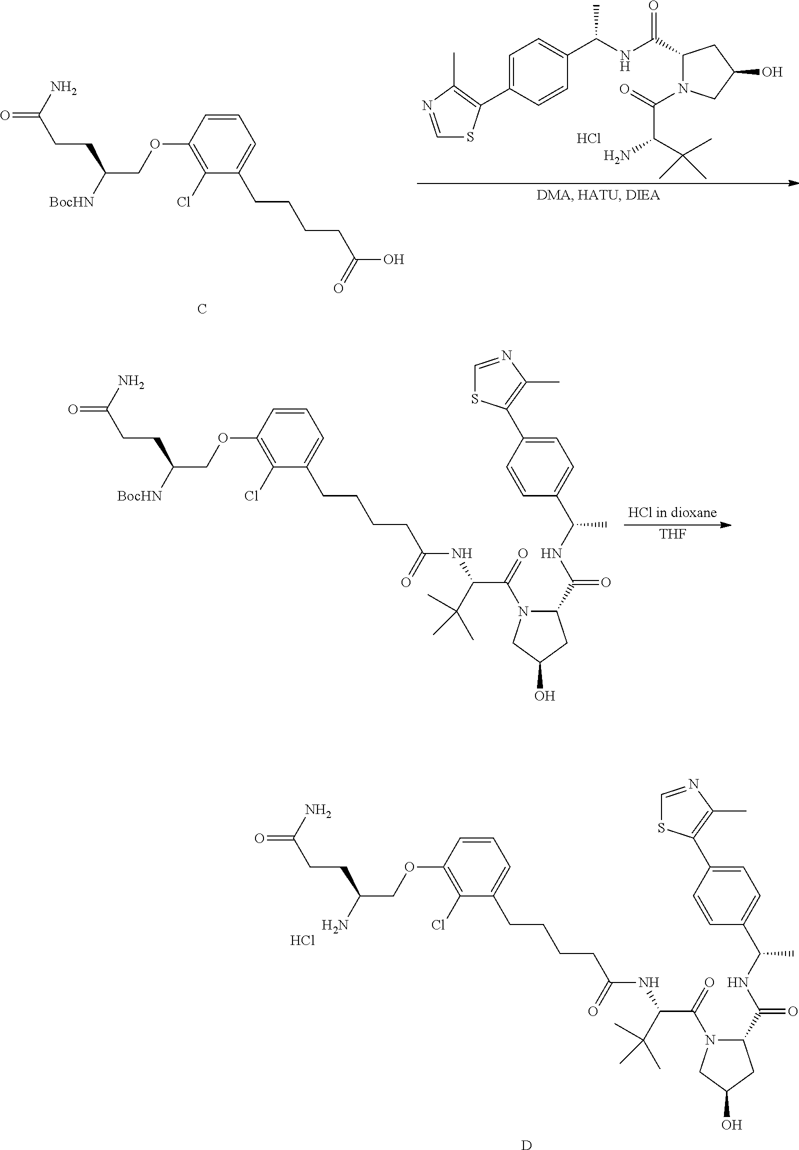

Step 1—Tert-butyl N-[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate. To a solution of 5-[3-[(2S)-2-[(tert-butoxycarbonyl)amino]-4-carbamoylbutoxy]-2-chlorophenyl]pentanoic acid (3.30 g, 7.45 mmol, Intermediate C) in DMA (40 mL) were added HATU (3.40 g, 8.94 mmol), TEA (2.26 g, 22.35 mmol), (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (4.30 g, 8.94 mmol, CAS #1448189-80-7) at rt under nitrogen atmosphere and the mixture was stirred for 16 h. On completion, the resulting mixture was concentrated under reduced pressure and the residue was purified by reverse phase flash chromatography (Column, C18 silica gel; mobile phase, MeCN in water (plus 10 mmol/L NH₄HCO₃), 40% to 60% gradient in 25 min; Detector, UV 254/220 nm, desired fractions were collected at 53% B) concentrated and lyophilized to afford the title compound (6.3 g, 99% yield) as a white solid. ¹H NMR (400 MHz, DMSO-d₆) δ 8.99 (s, 1H), 8.39 (d, J=7.8 Hz, 1H), 7.85 (d, J=9.2 Hz, 1H), 7.45 (d, J=8.3 Hz, 2H), 7.39 (d, J=8.3 Hz, 2H), 7.30 (d, J=15.6 Hz, 1H), 7.20 (t, J=7.9 Hz, 1H), 6.98 (d, J=8.2 Hz, 1H), 6.91 (dd, J=7.6, 1.3 Hz, 1H), 6.81 (d, J=8.5 Hz, 1H), 6.75 (s, 1H), 5.11 (d, J=3.5 Hz, 1H), 4.99-4.87 (m, 1H), 4.53 (d, J=9.3 Hz, 1H), 4.44 (t, J=8.0 Hz, 1H), 4.31-4.27 (m, 1H), 3.93 (d, J=6.1 Hz, 2H), 3.83-3.73 (m, 1H), 3.67-3.56 (m, 2H), 2.71-2.67 (m, 2H), 2.47 (s, 3H), 2.36-2.26 (m, 1H), 2.22-2.10 (m, 3H), 2.10-1.97 (m, 1H), 1.92-1.75 (m, 2H), 1.70-1.49 (m, 4H), 1.40 (s, 9H), 1.38-1.34 (m, 2H), 1.31-1.22 (m, 2H), 0.94 (s, 9H); LC/MS (ESI, m/z): [(M+H)]⁺=869.4.

Step 2—(2S,4R)-1-[(2S)-2-(5-[3-[(2S)-2-amino-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-chloro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate (3.30 g, 3.79 mmol) in THF (30 mL) was added 4 M HCl (gas) in 1,4-dioxane (30 mL) dropwise at rt under nitrogen atmosphere and the mixture was stirred for 2 h at rt. On completion, the reaction mixture was concentrated under reduced pressure to afford the title compound (2.5 g, 82% yield) as a light yellow solid. ¹H NMR (400 MHz, DMSO-d₆) δ 9.06 (s, 1H), 8.41 (d, J=7.7 Hz, 1H), 8.36 (s, 2H), 7.84 (d, J=9.3 Hz, 1H), 7.45 (d, J=8.3 Hz, 2H), 7.39 (d, J=8.3 Hz, 2H), 7.25 (t, J=7.9 Hz, 1H), 7.05 (dd, J=8.3, 1.4 Hz, 1H), 7.02-6.96 (m, 1H), 4.93 (p, J=7.0 Hz, 1H), 4.52 (d, J=9.3 Hz, 1H), 4.43 (t, J=8.0 Hz, 1H), 4.31-4.20 (m, 1H), 4.15 (dd, J=10.5, 5.6 Hz, 1H), 3.69-3.57 (m, 3H), 2.70 (d, J=7.1 Hz, 2H), 2.47 (s, 3H), 2.41-2.23 (m, 2H), 2.23-2.11 (m, 1H), 2.07-1.91 (m, 4H), 1.85-1.72 (m, 3H), 1.63-1.60 (m, 1H), 1.59-1.51 (m, 5H), 1.38 (d, J=7.0 Hz, 3H), 0.94 (s, 9H); LC/MS (ESI, m z): [(M+1]⁺=769.3.

### Methyl 4-[3-[(2S)-2-[(tert-butoxycarbonyl)amino]-4-carbamoylbutoxy]-2-chlorophenyl]butanoic acid (Intermediate E)

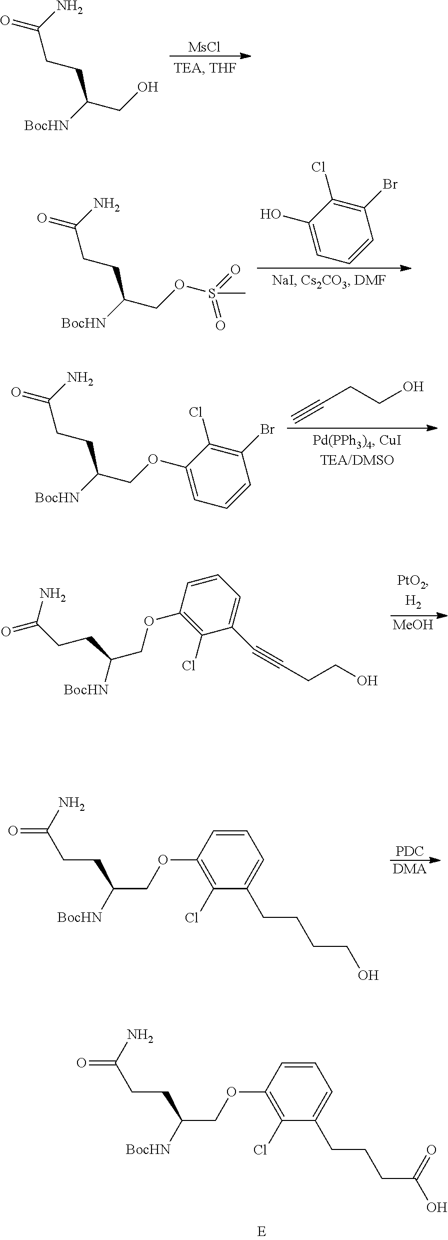

Step 1—Tert-butyl N-[(2S)-4-carbamoyl-1-(methanesulfonyloxy)butan-2-yl]carbamate. To a stirred mixture of tert-butyl N-[(2S)-4-carbamoyl-1-hydroxybutan-2-yl]carbamate (280.00 g, 1205.44 mmol, CAS #133565-42-1) and TEA (335.11 mL, 3311.65 mmol) in THF (5.00 L) was added MsCl (207.13 g, 1808.16 mmol) dropwise at 0° C. under nitrogen atmosphere. The resulting mixture was stirred for 16 h at rt under nitrogen atmosphere. On completion, the mixture was concentrated under reduced pressure. Then the mixture was diluted with ethyl acetate (5 L), and washed with of 0.2 M aq. HCl (2 L×2). The water phase was re-extracted with EtOAc (3×3 L). The combined organic layers were washed with brine (3 L×2) and dried over anhydrous Na₂SO₄. After filtration, the filtrate was concentrated under reduced pressure afford the title compound (345 g, 92%) as a white solid. ¹H NMR (400 MHz, Chloroform-d) δ 6.10 (s, 1H), 5.67 (s, 1H), 5.04 (d, J=8.7 Hz, 1H), 4.36-4.22 (m, 2H), 3.97-3.92 (m, 1H), 3.07 (s, 3H), 2.46-2.34 (m, 2H), 2.09-1.82 (m, 2H), 1.46 (s, 9H). LC/MS (ESI, m z): [(M+H)]⁺=311.1.

Step 2—Tert-butyl N-[(2S)-1-(3-bromo-2-chlorophenoxy)-4-carbamoylbutan-2-yl]carbamate. To a stirred solution of tert-butyl N-[(2S)-4-carbamoyl-1-(methanesulfonyloxy)butan-2-yl]carbamate (225.00 g, 724.94 mmol) and 3-bromo-2-chlorophenol (150.39 g, 724.94 mmol) in DMF (2.25 L) were added NaI (54.33 g, 362.47 mmol) and Cs₂CO₃ (472.40 g, 1449.88 mmol) in portions at rt under nitrogen atmosphere. The resulting mixture was stirred for 16 h at 70° C. under nitrogen atmosphere. On completion, the reaction mixture was cooled to rt and filtered. The filter cake was washed with ethyl acetate (4×500 mL). The filtrate was diluted with ethyl acetate (3 L) and the resulting mixture was washed with water (3×1 L) and brine (1 L×3), then dried over anhydrous Na₂SO₄. After filtration, the filtrate was concentrated under reduced pressure. The residue was purified by silica gel column chromatography, eluted with CH₂Cl₂/MeOH (30:1) to afford the title compound (139 g, 45% yield) as a white solid. ¹H NMR (400 MHz, Chloroform-d) δ 7.21 (dd, J=8.1, 1.3 Hz, 1H), 7.04 (t, J=8.2 Hz, 1H), 6.83 (dd, J=8.3, 1.4 Hz, 1H), 6.43 (s, 1H), 5.85 (s, 1H), 5.24 (d, J=8.7 Hz, 1H), 4.02-3.98 (m, 3H), 2.33 (t, J=7.0 Hz, 2H), 2.05-1.93 (m, 2H), 1.41 (s, 9H). LC/MS (ESI, m z): [(M+H)]⁺=421.1, 423.1.

Step 3—tert-butyl N-[(2S)-4-carbamoyl-1-[2-chloro-3-(4-hydroxybut-1-yn-1-yl)phenoxy]butan-2-yl]carbamate. To a stirred solution of tert-butyl N-[(2S)-1-(3-bromo-2-chlorophenoxy)-4-carbamoylbutan-2-yl]carbamate (156.00 g, 369.91 mmol) in DMSO (1.60 L) were added 3-butyn-1-ol (77.78 g, 1109.74 mmol) and TEA (800.00 mL) dropwise at rt under nitrogen atmosphere. To the above mixture were added CuI (7.05 g, 36.99 mmol) and Pd(PPh₃)₄ (42.75 g, 36.99 mmol) in portions at rt. The resulting mixture was stirred for 4 h at 85° C. under nitrogen atmosphere. On completin, the reaction mixture was allowed to cool to rt and concentrated under reduced pressure to removed TEA. The resulting mixture was filtered and the filter cake was washed with ethyl acetate (4×500 mL). The filtrate was diluted with water (3 L) and extracted with EtOAc (4×4 L). The combined organic layers were washed with brine (4×3 L), and dried over anhydrous Na₂SO₄. After filtration, the filtrate was concentrated under reduced pressure. The residue was purified by silica gel column chromatography, eluted with CH₂Cl₂/MeOH (60:1) to afford the title compound (130 g, 85% yield) as a brown solid. ¹H NMR (300 MHz, DMSO-d₆) δ 7.28-7.22 (m, 2H), 7.16-7.08 (m, 2H), 6.82 (d, J=8.4 Hz, 1H), 6.74 (s, 1H), 3.96 (d, J=6.2 Hz, 2H), 3.81-3.74 (m, 1H), 3.61 (t, J=6.9 Hz, 2H), 3.22-3.16 (m, 1H), 2.62 (t, J=6.9 Hz, 2H), 2.20-2.08 (m, 2H), 1.85-1.81 (m, 1H), 1.65-1.61 (m, 1H), 1.40 (s, 9H). LC/MS (ESI, m z): [(M+H)]⁺=411.2.

Step 4—tert-butyl N-[(2S)-4-carbamoyl-1-[2-chloro-3-(4-hydroxybutyl)phenoxy]butan-2-yl]carbamate. To a stirred solution of tert-butyl N-[(2S)-4-carbamoyl-1-[2-chloro-3-(4-hydroxybut-1-yn-1-yl)phenoxy]butan-2-yl]carbamate (128.00 g, 311.51 mmol) in MeOH (1.30 L) was added PtO₂ (7.07 g, 31.15 mmol) in portions at rt under nitrogen atmosphere. The reaction system was degassed under vacuum and purged with H₂ several times, then the mixture was hydrogenated under H₂ balloon (1 atm) at 25° C. for 3 h. On completion, PtO₂ was filtered off through celite and the filter cake was washed with MeOH (3×1 L). The filtrate was concentrated under reduced pressure and the residue was purified by silica gel column chromatography, eluted with PE/EtOAc (1:2) to afford the title compound (108 g, 83% yield) as a brown solid. ¹H NMR (300 MHz, DMSO-d₆) δ 7.28-7.22 (m, 2H), 7.16-7.08 (m, 2H), 6.82 (d, J=8.4 Hz, 1H), 6.74 (s, 1H), 3.96 (d, J=6.2 Hz, 2H), 3.81-3.74 (m, 1H), 3.61 (t, J=6.9 Hz, 2H), 3.22-3.16 (m, 1H), 2.62 (t, J=6.9 Hz, 2H), 2.20-2.08 (m, 2H), 1.85-1.81 (m, 1H), 1.65-1.61 (m, 1H), 1.40 (s, 9H). LC/MS (ESI, m/z): [(M+H)]⁺=415.2.

Step 5—methyl 4-[3-[(2S)-2-[(tert-butoxycarbonyl)amino]-4-carbamoylbutoxy]-2-chlorophenyl]butanoic acid. To a stirred solution of tert-butyl N-[(2S)-4-carbamoyl-1-[2-chloro-3-(4-hydroxybutyl)phenoxy]butan-2-yl]carbamate (107 g, 257.8 mmol) in DMA (1.00 L) was added PDC (484.6 g, 1289 mmol) in portions at rt under air atmosphere. The resulting mixture was stirred for 16 h at rt under air atmosphere. On completion, mixture was filtered, and the filter cake was washed with water (3×1 L) and EtOAc (2×2 L). The mixture was then extracted with EtOAc (2×2 L). The combined organic layers were washed with brine (5×1 L), and dried over anhydrous Na₂SO₄. After filtration, the filtrate was concentrated under reduced pressure and the residue was purified by silica gel column chromatography, eluted with CH₂Cl₂/MeOH (40:1) to afford 85 g of crude product. The crude was dissolved in EA (2000 mL) and washed with 1 M aq. NaOH (2×500 mL). The aqueous phase was adjusted to pH=4 with 1 N HCl and extracted with EA (4×1000 mL). The combined organic layers were washed with brine (2×500 mL), dried over anhydrous Na₂SO₄. After filtration, the filtrate was concentrated under reduced pressure to afford the title compound (58 g, 52% yield) as a white solid. ¹H NMR (400 MHz, DMSO-d₆) δ 12.08 (s, 1H), 7.29 (s, 1H), 7.20 (t, J=7.9 Hz, 1H), 6.99 (d, J=8.1 Hz, 1H), 6.89 (dd, J=7.6, 1.3 Hz, 1H), 6.80 (d, J=8.5 Hz, 1H), 6.75 (s, 1H), 3.93 (d, J=6.1 Hz, 2H), 3.82-3.75 (m, 2H), 2.74-2.68 (m, 2H), 2.26 (t, J=7.4 Hz, 1H), 2.18-2.12 (m, 2H), 1.92-1.78 (m, 3H), 1.67-1.56 (m, 1H), 1.39 (s, 9H); LC/MS (ESI, m/z): [(M+H)]⁺=415.2.

### (2S,4R)-1-((S)-2-(4-(2-chloro-3-(((S)-2,5-diamino-5-oxopentyl)oxy)phenyl)butanamido)-3,3-dimethylbutanoyl)-4-hydroxy-N—((S)-1-(4-(4-methylthiazol-5-yl)phenyl)ethyl)pyrrolidine-2-carboxamide hydrochloride (Intermediate F)

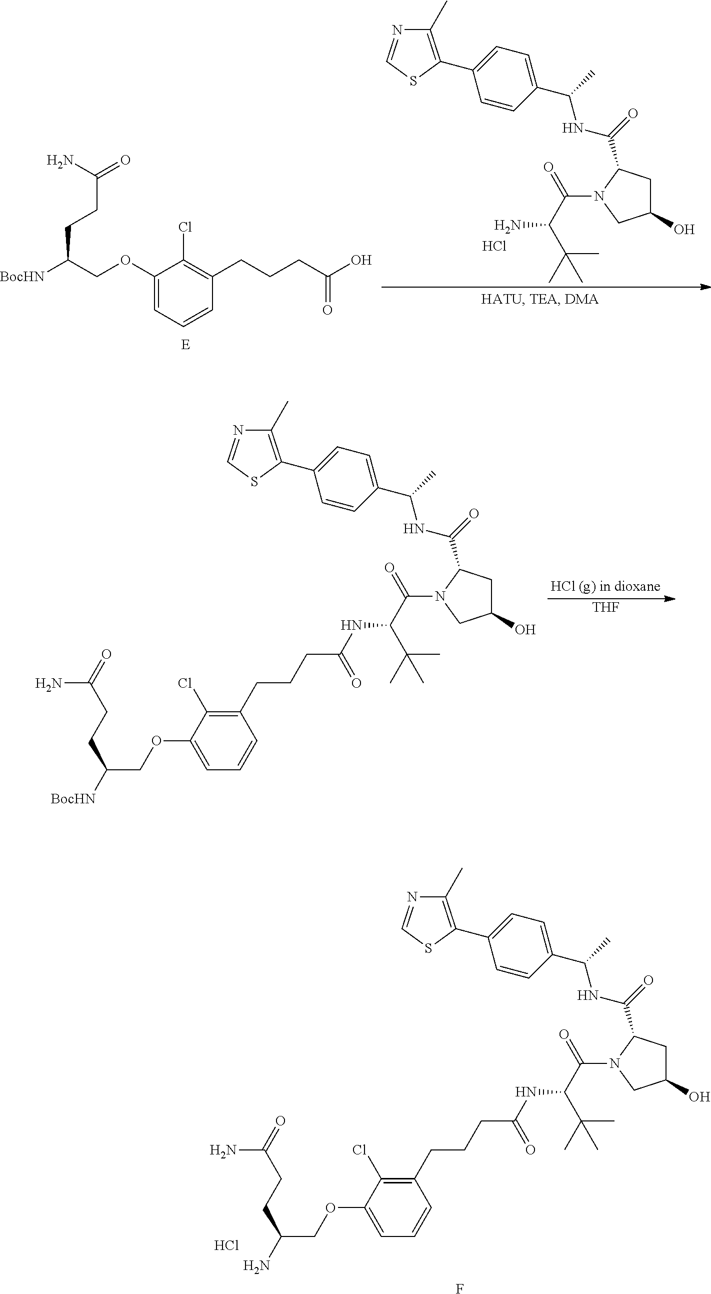

Step 1—Tert-butyl N-[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate. To a stirred mixture of 4-[3-[(2S)-2-[(tert-butoxycarbonyl)amino]-4-carbamoylbutoxy]-2-chlorophenyl]butanoic acid (4.00 g, 9.33 mmol, Intermediate E) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (5.38 g, 11.19 mmol, CAS #1448189-80-7) in DMA (40.00 mL) were added HATU (5.32 g, 13.99 mmol) and TEA (2.83 g, 27.98 mmol) in portions at rt under air atmosphere. The resulting mixture was stirred for 1 h at room temperature under air atmosphere. On completion, the residue was purified by reverse phase flash chromatography (Column: WelFlash™ C18-I, 20-40 μm, 330 g; EluentA: Water (plus 10 mmol/L FA); Eluent B: ACN; Gradient: 35%-55% B in 25 min; Flow rate: 80 mL/min; Detector: 220/254 nm; desired fractions were collected at 51% B) to afford the title compound (7 g, 88% yield) as a white solid. ¹H NMR (400 MHz, DMSO-d₆) δ 8.99 (s, 1H), 8.39 (d, J=7.8 Hz, 1H), 7.89 (d, J=9.3 Hz, 1H), 7.47-7.36 (m, 4H), 7.29 (s, 1H), 7.20 (t, J=8.0 Hz, 1H), 6.98 (dd, J=8.3, 1.4 Hz, 1H), 6.89 (dd, J=7.7, 1.4 Hz, 1H), 6.81 (d, J=8.4 Hz, 1H), 6.75 (s, 1H), 5.11 (d, J=3.5 Hz, 1H), 4.96-4.88 (m, 1H), 4.54 (d, J=9.3 Hz, 1H), 4.44 (t, J=8.0 Hz, 1H), 4.31-4.27 (m, 1H), 3.93 (d, J=6.2 Hz, 2H), 3.82-3.74 (m, 1H), 3.65-3.60 (m, 2H), 3.36 (s, 4H), 2.73-2.63 (m, 2H), 2.46 (s, 3H), 2.37-2.25 (m, 1H), 2.23-2.10 (m, 2H), 1.89-1.71 (m, 2H), 1.69-1.55 (m, 1H), 1.39-1.33 (m, 12H), 0.95 (s, 9H). LC/MS (ESI, m/z): [(M+H)]⁺=855.4.

Step 2—(2S,4R)-1-((S)-2-(4-(2-chloro-3-(((S)-2,5-diamino-5-oxopentyl)oxy)phenyl)butanamido)-3,3-dimethylbutanoyl)-4-hydroxy-N—((S)-1-(4-(4-methylthiazol-5-yl)phenyl)ethyl)pyrrolidine-2-carboxamide hydrochloride. To a stirred mixture of tert-butyl N-[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate (6.90 g, 8.07 mmol) in DCM (50.00 mL) was added HCl (gas) in 1,4-dioxane (20.00 mL) dropwise at rt. The resulting mixture was stirred for 1 h at rt under air atmosphere. On completion, the resulting mixture was concentrated under reduced pressure to afford the title compound (6.1 g, crude) as a white solid. ¹H NMR (300 MHz, DMSO-d₆) δ 9.31 (d, J=2.0 Hz, 1H), 8.50-8.46 (m, 4H), 7.91 (d, J=9.1 Hz, 1H), 7.52-7.38 (m, 4H), 7.26 (t, J=7.9 Hz, 1H), 7.07 (dd, J=8.3, 1.4 Hz, 1H), 6.97 (dd, J=7.7, 1.3 Hz, 1H), 4.98-4.88 (m, 1H), 4.54 (d, J=9.3 Hz, 1H), 4.45 (t, J=8.0 Hz, 1H), 4.30-4.17 (m, 3H), 3.62 (d, J=3.3 Hz, 2H), 2.70 (t, J=7.4 Hz, 2H), 2.50 (s, 3H), 2.37-2.28 (m, 4H), 2.25-2.15 (m, 1H), 2.09-1.92 (m, 4H), 1.84-1.73 (m, 4H), 1.39 (d, J=7.0 Hz, 3H), 0.96 (s, 9H). LC/MS (ESI, m z): [(M+H)]⁺=755.4.

### (2S)-1-[(2S)-2-[(tert-butoxycarbonyl)amino]-3-hydroxypropanoyl]pyrrolidine-2-carboxylic acid (Intermediate G)

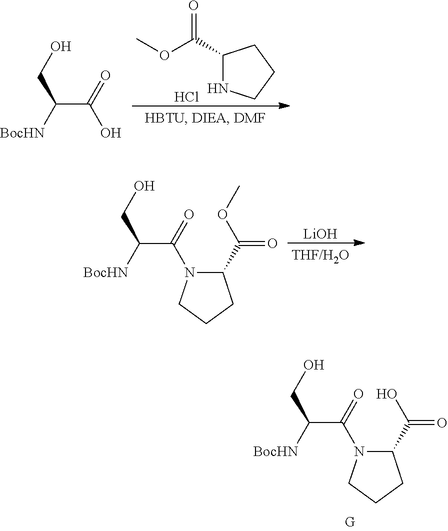

Step 1—Methyl (2S)-1-[(2S)-2-[(tert-butoxycarbonyl)amino]-3-hydroxypropanoyl]pyrrolidine-2-carboxylate. To a stirred solution of (2S)-2-[(tert-butoxycarbonyl)amino]-3-hydroxypropanoic acid (5.00 g, 24.37 mmol, CAS #204191-40-2) and methyl (2S)-pyrrolidine-2-carboxylate (3.78 g, 29.27 mmol) in DMF (50.00 mL) were added DIEA (12.60 g, 97.46 mmol) and HBTU (11.09 g, 29.24 mmol) in portions at 25° C. under nitrogen atmosphere. The resulting mixture was stirred for 2 h at rt under nitrogen atmosphere. On completion, the reaction mixture was diluted with water (200 mL) and extracted with EtOAc (3×300 mL). The combined organic layers were washed with brine (2×200 mL), and dried over anhydrous Na₂SO₄. After filtration, the filtrate was concentrated under reduced pressure and the crude product was purified by reverse phase flash (Column: Spherical C18 Column, 20-40 um, 330 g; Mobile Phase A: Water (0.1% FA), Mobile Phase B: ACN; Flow rate: 100 mL/min; Gradient: 35% B to 55% B in 25 min, 254 nm, t fractions containing the desired product were collected at 52% B) to afford the title compound (5.4 g, 70% yield) as a white solid. ¹H NMR (400 MHz, Methanol-d₄) δ 4.55-4.48 (m, 2H), 3.88-3.75 (m, 3H), 3.72 (s, 3H), 3.66-3.61 (m, 1H), 2.36-2.23 (m, 1H), 2.12-1.93 (m, 3H), 1.46 (s, 9H); LC/MS (ESI, m z): [(M+1)]⁺=317.1.

Step 2—(2S)-1-[(2S)-2-[(tert-butoxycarbonyl)amino]-3-hydroxypropanoyl]pyrrolidine-2-carboxylic acid. To a stirred solution of methyl (2S)-1-[(2S)-2-[(tert-butoxycarbonyl)amino]-3-hydroxypropanoyl]pyrrolidine-2-carboxylate (5.40 g, 17.07 mmol) in THF (25.00 mL) was added a solution of LiOH (3.27 g, 136.55 mmol) in H₂O (25.00 mL) dropwise at rt under nitrogen atmosphere and the mixture was stirred for 2 h at rt. On completion, the reaction mixture was concentrated under reduced pressure. The crude product was purified by reverse phase flash (Column: Spherical C18 Column, 20-40 um, 330 g; Mobile Phase A: Water (0.1% FA), Mobile Phase B: ACN; Flow rate: 100 mL/min; Gradient: 25% B to 50% B in 25 min, 254 nm, the fractions containing the desired product were collected at 33% B) to afford the title compound (1.7 g, 33% yield) as a white solid. ¹H NMR (400 MHz, Methanol-d₄) δ 5.13-5.04 (m, 2H), 4.50-4.31 (m, 3H), 4.28-4.18 (m, 1H), 3.00-2.91 (m, 1H), 2.77-2.60 (m, 3H), 2.18 (s, 9H); LC/MS (ESI, m z): [(M+1)]⁺=303.2.

### (5S,8S,10aR)-3-acetyl-5-[(tert-butoxycarbonyl)amino]-6-oxo-octahydropyrrolo[1,2-a][1,5]diazocine-8-carboxylic acid (Intermediate H)

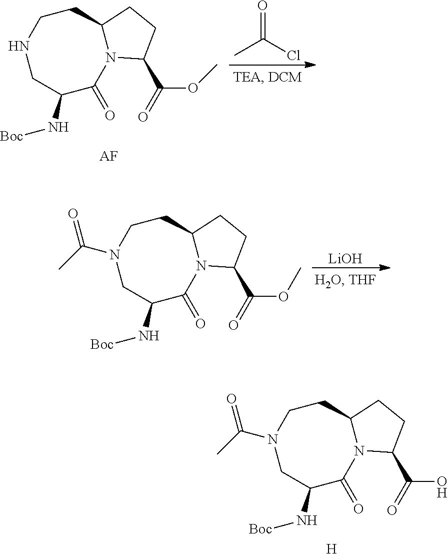

Step 1—Methyl (5S,8S,10aR)-3-acetyl-5-[(tert-butoxycarbonyl)amino]-6-oxo-octahydropyrrolo[1,2-a][1,5]diazocine-8-carboxylate. To a solution of methyl (5S,8S,10aR)-5-[(tert-butoxycarbonyl)amino]-6-oxo-octahydro-1H-pyrrolo[1,2-a][1,5]diazocine-8-carboxylate (4.44 g, 13.01 mmol, Intermediate AF) in DCM (50.00 mL) were added TEA (3.95 g, 39.02 mmol) and acetyl chloride (1.53 g, 19.51 mmol) at 0° C., then the mixture was stirred at rt for 3 hours. The reaction was quenched by the addition of sat. NaHCO₃ (100 mL) and the resulting mixture was extracted with DCM (5×100 mL). The combined organic layers were concentrated under reduced pressure. The residue was purified by reverse phase flash chromatography (column, C¹⁸ silica gel; mobile phase, CH₃CN in water (plus 10 mmol/L NH₄HCO₃), 25% to 40% gradient in 15 min; Detector, UV 220/254 nm) to give the title compound as a light yellow solid (4.55 g, 91% yield): ¹H NMR (400 MHz, CDCl₃) δ 5.84 (d, J 6.4 Hz, 1H), 4.50 (t, J=8.6 Hz, 2H), 4.17-4.13 (m, 1H), 3.97-3.83 (m, 2H), 3.77 (s, 3H), 3.42-3.28 (m, 1H), 3.21 (dd, J 14.3, 10.7 Hz, 1H), 2.42-2.33 (m, 1H), 2.30 (s, 3H), 2.24-1.97 (m, 3H), 1.90-1.72 (m, 2H), 1.44 (s, 9H). LC/MS (ESI, m z): [(M+1)]⁺=384.1.

Step 2—(5S,8S,10aR)-3-acetyl-5-[(tert-butoxycarbonyl)amino]-6-oxo-octahydropyrrolo[1,2-a][1,5]diazocine-8-carboxylic acid. To a stirred solution of methyl (5S,8S,10aR)-3-acetyl-5-[(tert-butoxycarbonyl)amino]-6-oxo-octahydropyrrolo[1,2-a][1,5]diazocine-8-carboxylate (4.5 g) in THF (50.0 mL) was added H₂O (50.0 mL) and LiOH (5.29 g, 221 mmol) in portions at rt and the resulting mixture was stirred for 16 h. The mixture was acidified to pH 6 with aqueous HCl (1 M). The precipitated solids were collected by filtration and washed with water (2×10.0 mL). Then it was dried in vacuum to give the title compound as a white solid. (300 MHz, CDCl₃) δ 5.87 (d, J=6.9 Hz, 1H), 4.67-4.65 (m, 1H), 4.54 (t, J=8.3 Hz, 1H), 4.29-4.25 (m, 1H), 3.95-3.77 (m, 2H), 3.68-3.63 (m, 3H), 3.35 (t, J=12.5 Hz, 1H), 2.36-2.34 (m, 2H), 2.23 (s, 3H), 2.01-1.98 (s, 1H), 1.84-1.80 (m, 1H), 1.45 (s, 9H). LC/MS (ESI, m z): [(M+1)]⁺=370.2.

### (2S,4R)-1-[(2R)-2-[3-(3-[3-[(2S)-2-amino-4-carbamoylbutoxy]-2-chlorophenyl]propoxy)-1,2-oxazol-5-yl]-3-methylbutanoyl]-4-hydroxy-N-[[4-(4-methyl-1,3-thiazol-5-yl)phenyl]methyl]pyrrolidine-2-carboxamide hydrochloride (Intermediate I)

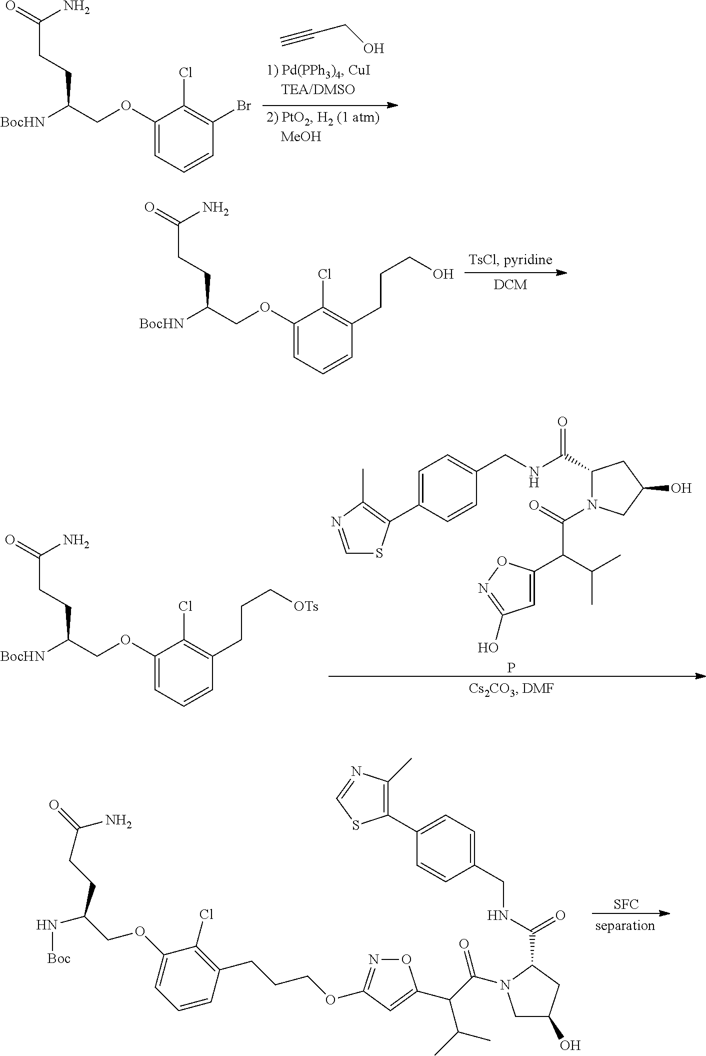

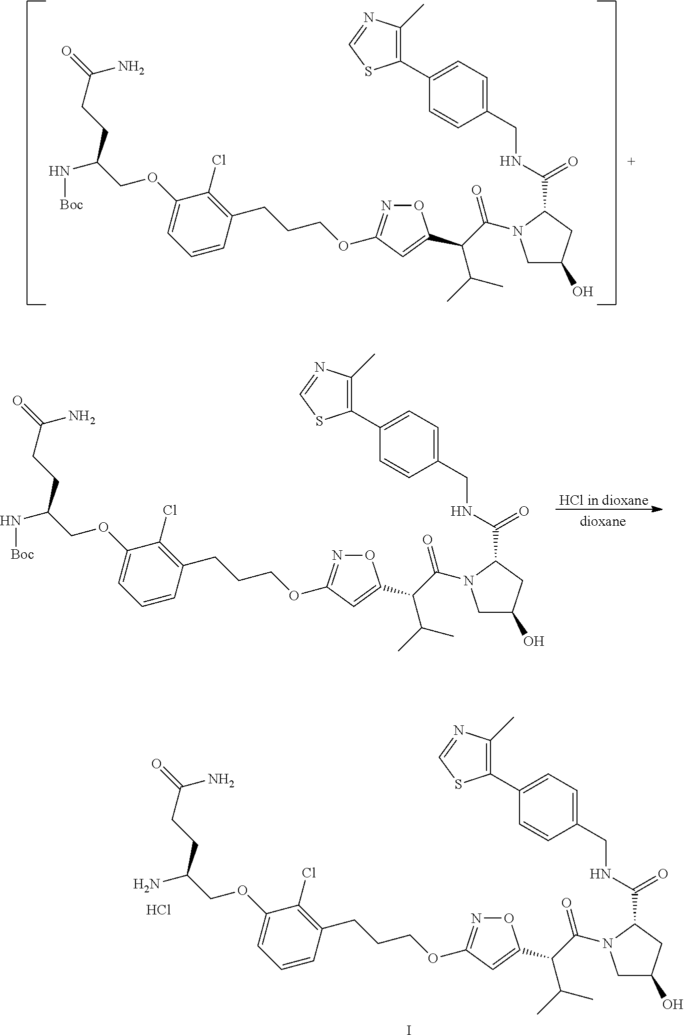

Step 1—Tert-butyl N-[(2S)-4-carbamoyl-1-[2-chloro-3-(3-hydroxyprop-1-yn-1-yl)phenoxy]butan-2-yl]carbamate. To a stirred solution of tert-butyl N-[(2S)-1-(3-bromo-2-chlorophenoxy)-4-carbamoylbutan-2-yl]carbamate (2.00 g, 4.74 mmol, synthesized via Steps 1-3 of Intermediate C) and propargyl alcohol (797.65 mg, 14.23 mmol) in DMSO (8.00 mL) were added TEA (8.57 mL, 84.69 mmol), Pd(PPh₃)₄ (548.02 mg, 0.47 mmol) and CuI (90.32 mg, 0.47 mmol) in turns at rt under nitrogen atmosphere. The reaction mixture was then stirred for 2 h at 80° C. under nitrogen atmosphere. On completion, the mixture was cooled to rt and filtered. The filter cake was washed with MeCN (2×5 mL) and the filtrate was concentrated under reduced pressure. The solution was purified by reverse phase flash chromatography (Column: WelFlash™ C¹⁸-I, 20-40 um, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 51% B) and concentrated under reduced pressure to afford the title compound (1.3122 g, 70% yield) as a yellow solid. ¹H NMR (400 MHz, DMSO-d₆) δ 7.30-7.26 (m, 2H), 7.19-7.16 (m, 1H), 7.13-7.11 (m, 1H), 6.83 (d, J=8.5 Hz, 1H), 6.74 (s, 1H), 5.42-5.39 (m, 1H), 4.37-4.33 (m, 2H), 4.02-3.90 (m, 2H), 3.83-3.70 (m, 1H), 2.22-2.04 (m, 2H), 1.90-1.78 (m, 1H), 1.68-1.55 (m, 1H), 1.39 (s, 9H); LC/MS (ESI, m z): [(M+1)]⁺=397.1.

Step 2—Tert-butyl N-[(2S)-4-carbamoyl-1-[2-chloro-3-(3-hydroxypropyl)phenoxy]butan-2-yl]carbamate. To a stirred solution of tert-butyl N-[(2S)-4-carbamoyl-1-[2-chloro-3-(3-hydroxyprop-1-yn-1-yl)phenoxy]butan-2-yl]carbamate (1.31 g, 3.30 mmol) in MeOH (20 mL) was added PtO₂ (74.96 mg, 0.33 mmol) in portions at rt under nitrogen atmosphere. The resulting mixture was stirred for 2 h at rt under hydrogen atmosphere. On completion, the reaction mixture was filtered and the filter cake was washed with MeOH (3×10 mL). The filtrate was concentrated under reduced pressure to afford crude product. The residue was purified by reverse flash chromatography (column, C¹⁸ silica gel; mobile phase, MeCN in water (10 mmol/L NH₄HCO₃), 35% to 55% gradient in 20 min; detector, UV 254 nm) to afford the title compound (1.15 g, 87%) as a yellow solid. ¹H NMR (400 MHz, DMSO-d₆) δ 7.28 (s, 1H), 7.22-7.18 (m, 1H), 7.01-6.95 (m, 1H), 6.92-6.89 (m, 1H), 6.85-6.78 (m, 1H), 6.75 (s, 1H), 4.55-4.51 (m, 1H), 3.96-3.89 (m, 2H), 3.80-3.76 (m, 1H), 3.49-3.40 (m, 2H), 2.75-2.67 (m, 2H), 2.19-2.10 (m, 2H), 1.91-1.81 (m, 1H), 1.70-1.66 (m, 3H), 1.40 (s, 9H); LC/MS (ESI, m z): [(M+1)]⁺=401.2.

Step 3—Tert-butyl N-[(2S)-4-carbamoyl-1-(2-chloro-3-[3-[(4-methylbenzenesulfonyl)oxy]propyl]phenoxy)butan-2-yl]carbamate. To a stirred mixture of tert-butyl N-[(2S)-4-carbamoyl-1-[2-chloro-3-(3-hydroxypropyl)phenoxy]butan-2-yl]carbamate (1.15 g, 2.87 mmol) and pyridine (453.80 mg, 5.74 mmol) in DCM (15.00 mL) was added a solution of p-toluenesulfonyl chloride (820.29 mg, 4.30 mmol) in DCM (5 mL) dropwise at 0° C. under nitrogen atmosphere. The resulting mixture was stirred for 16 h at rt under nitrogen atmosphere. On completion, the reaction was quenched by the addition of water (5 mL) at 0° C. The resulting mixture was extracted with CH₂Cl₂ (4×20 mL). The combined organic layers were washed with brine (2×10 mL), and dried over anhydrous Na₂SO₄. After filtration, the filtrate was concentrated under reduced pressure and the residue was purified by silica gel column chromatography, eluted with PE/EtOAc (1:5), to afford the title compound (427 mg, 27% yield) as a yellow solid. ¹H NMR (300 MHz, DMSO-d₆) δ 7.81 (d, J=7.9 Hz, 2H), 7.52-7.48 (m, 2H), 7.28 (s, 1H), 7.18-7.14 (m, 1H), 7.00-6.97 (m, 1H), 6.81-6.77 (m, 3H), 4.06 (t, J=6.2 Hz, 2H), 3.93 (d, J=6.1 Hz, 2H), 3.83-3.72 (m, 1H), 2.69-2.65 (m, 2H), 2.45 (s, 3H), 2.24-2.03 (m, 2H), 1.95-1.77 (m, 3H), 1.72-1.53 (m, 1H), 1.40 (s, 9H); LC/MS (ESI, m z): [(M+1)]⁺=555.2.

Step 4—Tert-butyl N-[(2S)-4-carbamoyl-1-(2-chloro-3-[3-[(5-[1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl)oxy]propyl]phenoxy)butan-2-yl]carbamate. To a stirred solution of tert-butyl N-[(2S)-4-carbamoyl-1-(2-chloro-3-[3-[(4-methylbenzenesulfonyl)oxy]propyl]phenoxy)butan-2-yl]carbamate (390.00 mg, 0.70 mmol,) and (2S,4R)-4-hydroxy-1-[2-(3-hydroxy-1,2-oxazol-5-yl)-3-methylbutanoyl]-N-[[4-(4-methyl-1,3-thiazol-5-yl)phenyl]methyl]pyrrolidine-2-carboxamide (408.55 mg, 0.84 mmol, Intermediate P) in DMF (5.00 mL) was added Cs₂CO₃ (457.84 mg, 1.41 mmol) in portions at rt under nitrogen atmosphere and the mixture was stirred for 16 h. On completion, the residue was purified by reverse flash chromatography (column, C¹⁸ silica gel; mobile phase, MeCN in water (10 mmol/L NH₄HCO₃), 40% to 65% gradient in 25 min; detector, UV 220 nm) to afford the title compound (478 mg, 78% yield) as a white solid. ¹H NMR (300 MHz, DMSO-d₆) δ 9.03-8.99 (m, 1H), 8.54-8.50 (m, 1H), 7.51-7.30 (m, 4H), 7.22-7.20 (m, 1H), 7.04-7.00 (m, 1H), 6.93-6.89 (m, 1H), 6.84-6.80 (m, 1H), 6.75 (s, 1H), 6.15-6.06 (m, 1H), 5.17-5.13 (m, 1H), 4.38-4.32 (m, 3H), 4.22-4.10 (m, 1H), 3.95-3.90 (m, 2H), 3.80-3.78 (m, 2H), 3.60-3.57 (m, 1H), 3.36-3.32 (m, 2H), 2.86-2.74 (m, 3H), 2.54-2.41 (m, 3H), 2.31-2.24 (m, 1H), 2.18-2.11 (m, 3H), 2.10-1.76 (m, 4H), 1.65-1.58 (m, 2H), 1.40 (s, 9H), 0.87-0.83 (m, 6H); LC/MS (ESI, m z): [(M+1)]⁺=867.3.

Step 5—Tert-butyl N-[(2S)-4-carbamoyl-1-[2-chloro-3-[3-([5-[(2R)-1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl]oxy)propyl]phenoxy]butan-2-yl]carbamate. Tert-butyl N-[(2S)-4-carbamoyl-1-(2-chloro-3-[3-[(5-[1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl)oxy]propyl]phenoxy)butan-2-yl]carbamate (400.00 mg) was separated by SFC to afford pre-peak tert-butyl N-[(2S)-4-carbamoyl-1-[2-chloro-3-[3-([5-[(2S)-1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl]oxy)propyl]phenoxy]butan-2-yl]carbamate (189 mg, 40% yield) as a white solid and post-peak tert-butyl N-[(2S)-4-carbamoyl-1-[2-chloro-3-[3-([5-[(2R)-1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl]oxy)propyl]phenoxy]butan-2-yl]carbamate (209 mg, 44% yield) as a white solid. ¹H NMR (400 MHz, DMSO-d₆) δ 8.99-8.97 (m, 1H), 8.53 (t, J=6.0 Hz, 1H), 7.46-7.42 (m, 2H), 7.41-7.33 (m, 2H), 7.29-7.25 (m, 1H), 7.22-7.20 (m, 1H), 7.02-6.98 (m 1H), 6.96-6.87 (m, 1H), 6.81 (d, J=8.4 Hz, 1H), 6.74 (s, 1H), 6.11 (s, 1H), 5.12 (d, J=3.7 Hz, 1H), 4.42-4.24 (m, 4H), 4.21-4.07 (m, 2H), 3.95-3.91 (m, 2H), 3.84-3.71 (m, 2H), 3.68 (d, J=9.6 Hz, 1H), 3.50-3.37 (m, 1H), 2.83-2.71 (m, 2H), 2.46-2.42 (m, 3H), 2.35-2.19 (m, 1H), 2.17-2.08 (m, 2H), 2.08-1.95 (m, 3H), 1.96-1.78 (m, 2H), 1.64-1.60 (m, 1H), 1.39 (s, 9H), 0.96 (d, J=6.5 Hz, 3H), 0.81 (d, J=6.8, 1.9 Hz, 3H); LC/MS (ESI, m z): [(M+1)]⁺=867.3.

Step 6—(2S,4R)-1-[(2i)-2-[3-(3-[3-[(2S)-2-amino-4-carbamoylbutoxy]-2-chlorophenyl]propoxy)-1,2-oxazol-5-yl]-3-methylbutanoyl]-4-hydroxy-N-[[4-(4-methyl-1,3-thiazol-5-yl)phenyl]methyl]pyrrolidine-2-carboxamide hydrochloride. To a stirred solution of tert-butyl N-[(2S)-4-carbamoyl-1-[2-chloro-3-[3-([5-[(2R)-1-[(2S,4R)-4-hydroxy-2-([[4-(4-methyl-1,3-thiazol-5-yl)phenyl]methyl]carbamoyl)pyrrolidin-1-yl]-3-methyl-1-oxobutan-2-yl]-1,2-oxazol-3-yl]oxy)propyl]phenoxy]butan-2-yl]carbamate (209.00 mg, 0.24 mmol) in THF (5 mL) was added HCl (gas) in 1,4-dioxane (5 mL) dropwise at rt under nitrogen atmosphere and the mixture was stirred for 2 h. On completion, the resulting mixture was concentrated under reduced pressure to afford the title compound (186 mg, 96% yield) as a white solid. ¹H NMR (300 MHz, DMSO-d₆) δ 9.07-9.03 (m, 1H), 8.57 (s, 1H), 8.43-8.26 (m, 2H), 7.48-7.44 (m, 2H), 7.42-7.38 (m, 2H), 7.30-7.26 (m, 1H), 7.11-7.08 (m, 1H), 7.04-7.00 (m, 1H), 6.13 (d, J=1.1 Hz, 1H), 4.40-4.36 (m, 3H), 4.24-4.20 (m, 2H), 3.82-3.78 (m, 1H), 3.71-3.67 (m, 1H), 3.65-3.58 (m, 3H), 3.44-3.40 (m, 1H), 2.87-2.83 (m, 2H), 2.54-2.42 (m, 4H), 2.40-2.21 (m, 2H), 2.10-1.88 (m, 4H), 1.80-1.76 (m, 4H), 1.63-1.59 (m, 1H), 1.30-1.19 (m, 1H), 0.97 (d, J=6.5 Hz, 3H), 0.82 (d, J=6.6 Hz, 3H); LC/MS (ESI, m z): [(M+1)]⁺=767.2.

### (4S)-4-Amino-5-(4-methanesulfonylphenoxy)pentanamide hydrochloride (Intermediate J)

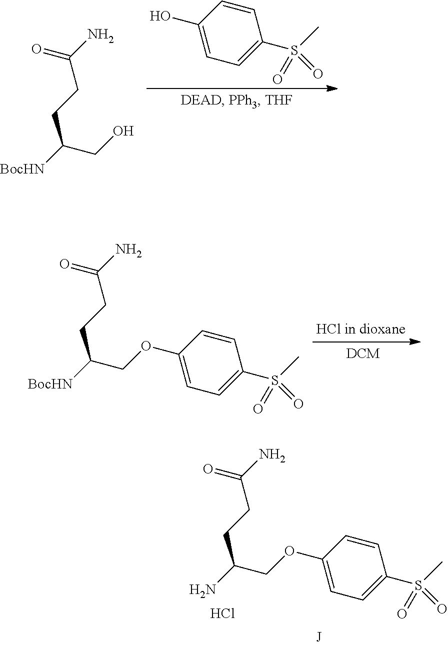

Step 1—Tert-butyl N-[(2S)-4-carbamoyl-1-(4-methanesulfonylphenoxy)butan-2-yl]carbamate. To a stirred mixture of tert-butyl N-[(2S)-4-carbamoyl-1-hydroxybutan-2-yl]carbamate (5.00 g, 21.53 mmol, CAS #133565-42-1), 4-methanesulfonylphenol (4.82 g, 27.98 mmol) and PPh₃ (6.78 g, 25.83 mmol) in THF (100.00 mL) was added DEAD (4.50 g, 25.83 mmol) dropwise at 0° C. under nitrogen atmosphere and the mixture was stirred for overnight at rt. On completion, the reaction mixture was concentrated under reduced pressure. The residue was purified by silica gel column chromatography, eluted with CH₂Cl₂/MeOH (10:1), to afford the title compound (1.7 g, 20% yield) as a yellow solid. ¹H NMR (300 MHz, Methanol-d₄) δ 7.92-7.85 (m, 2H), 7.20-7.13 (m, 2H), 4.13-4.03 (m, 2H), 3.97-3.92 (m, 1H), 3.19 (s, 3H), 2.42-2.26 (m, 2H), 2.13-1.95 (m, 1H), 1.90-1.71 (m, 1H), 1.46 (s, 9H); LC/MS (ESI, m/z): [(M−H)]⁻=385.1.

Step 2—(4S)-4-amino-5-(4-methanesulfonylphenoxy)pentanamide hydrochloride. To a stirred solution of tert-butyl N-[(2S)-4-carbamoyl-1-(4-methanesulfonylphenoxy)butan-2-yl]carbamate (1.70 g, 4.40 mmol) in DCM (20.00 mL) was added HCl (gas) in 1,4-dioxane (10.00 mL, 40.00 mmol) dropwise at rt under nitrogen atmosphere and the mixture was stirred for 1 h at rt. On completion, the reaction mixture was concentrated under vacuum to the title compound (1.3 g, 92%) as a light yellow solid. ¹H NMR (300 MHz, Methanol-d₄) δ 7.98-7.92 (m, 2H), 7.32-7.22 (m, 2H), 4.44-4.37 (m, 1H), 4.29-4.23 (m, 1H), 3.79-3.72 (m, 1H), 3.13 (s, 3H), 2.60-2.49 (m, 2H), 2.12-2.10 (m, 2H); LC/MS (ESI, m/z): [(M+H)]⁺=287.1.

### 5-[(Tert-butoxycarbonyl)amino]-6-oxo-octahydropyrrolo[1,2-a][1,5]diazocine-8-carboxylic acid (Intermediate K)

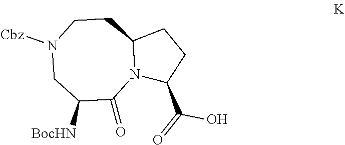 [SMILES: CC(C)(C)OC(=O)N[C@H]1CN(C(=O)OCc2ccccc2)CC[C@H]2CC[C@@H](C(=O)O)N2C1=O]

5-[(Tert-butoxycarbonyl)amino]-6-oxo-octahydropyrrolo[1,2-a][1,5]diazocine-8-carboxylic acid was synthesized as described in WO 2011050068.

### Tert-butyl N-[(5S,8S,10aR)-8-[[(2S)-4-carbamoyl-1-(4-methanesulfonylphenoxy)butan-2-yl]carbamoyl]-6-oxo-octahydro-1H-pyrrolo[1,2-a][1,5]diazocin-5-yl]carbamate (Intermediate L)

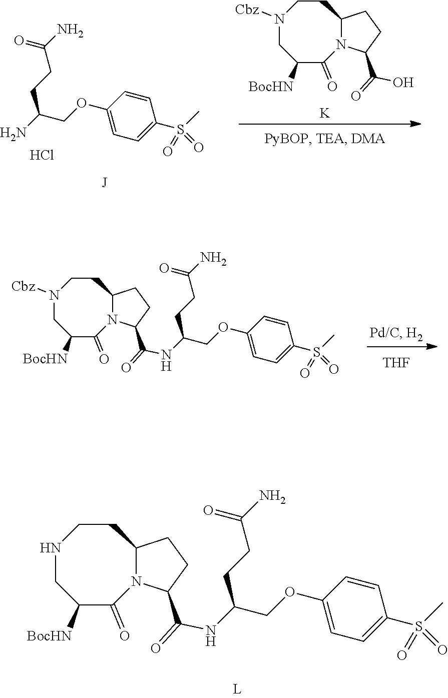

Step 1—Benzyl (5S,8S,10aR)-5-[(tert-butoxycarbonyl)amino]-8-[[(2S)-4-carbamoyl-1-(4-methanesulfonylphenoxy)butan-2-yl]carbamoyl]-6-oxo-octahydropyrrolo[1,2-a][1,5]diazocine-3-carboxylate. To a stirred solution of (4S)-4-amino-5-(4-methanesulfonylphenoxy)pentanamide hydrochloride (180.00 mg, 0.56 mmol, Intermediate J) and (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (334.56 mg, 0.73 mmol, Intermediate K) in DMA (5.00 mL) were added PyBOP (377.24 mg, 0.73 mmol) and TEA (225.70 mg, 2.23 mmol) dropwise at rt under nitrogen atmosphere and the mixture was stirred for 2 h. On completion, the reaction liquid was purified by reverse phase flash (Column: Spherical C18 Column, 20-40 um, 120 g; Mobile Phase A: Water (10 mmol/L NH₄HCO₃), Mobile Phase B: ACN; Flow rate: 60 mL/min; Gradient: 30% B to 50% B in 25 min, 254 nm, the fractions containing the desired product were collected at 46% B) to afford the title compound (210 mg, 52% yield) as a white solid. ¹H NMR (300 MHz, Methanol-d₄) δ 7.91-7.83 (m, 2H), 7.47-7.29 (m, 5H), 7.19-7.14 (m, 2H), 5.20 (s, 2H), 4.71-4.56 (m, 1H), 4.42-4.19 (m, 3H), 4.09-4.07 (m, 2H), 3.69-3.56 (m, 3H), 3.21-3.14 (m, 2H), 3.09 (s, 3H), 2.55-2.33 (m, 2H), 2.20-2.18 (m, 1H), 2.13-2.11 (m, 1H), 2.06-2.04 (m, 1H), 2.02-1.96 (m, 1H), 1.90-1.84 (m, 2H), 1.84-1.79 (m, 1H), 1.46 (s, 9H); LC/MS (ESI, m/z) [(M−H)]⁻=727.9.

Step 2—Tert-butyl N-[(5S,8S,10aR)-8-[[(2S)-4-carbamoyl-1-(4-methanesulfonylphenoxy)butan-2-yl]carbamoyl]-6-oxo-octahydro-1H-pyrrolo[1,2-a][1,5]diazocin-5-yl]carbamate. To a stirred solution of benzyl (5S,8S,10aR)-5-[(tert-butoxycarbonyl)amino]-8-[[(2S)-4-carbamoyl-1-(4-methanesulfonylphenoxy)butan-2-yl]carbamoyl]-6-oxo-octahydropyrrolo[1,2-a][1,5]diazocine-3-carboxylate (210.00 mg, 0.29 mmol) in THF (5.00 mL) was added Pd/C (80.00 mg, 0.75 mmol) at rt under nitrogen atmosphere and the reaction mixture was stirred for 2 h under hydrogen atmosphere. On completion, the resulting mixture was filtered, the filter cake was washed with MeOH (3×20 mL), and the filtrate was concentrated under reduced pressure. The crude product was purified by reverse phase flash (Column: Spherical C18 Column, 20-40 um, 120 g; Mobile Phase A: Water (10 mmol/L NH₄HCO₃), Mobile Phase B: ACN; Flow rate: 60 mL/min; Gradient: 30% B to 50% B in 25 min, 254 nm; the fractions containing the desired product were collected at 40% B) to afford the title compound (100 mg, 58%) as a white solid. ¹H NMR (300 MHz, Methanol-d₄) δ 7.93-7.77 (m, 2H), 7.18-7.10 (m, 2H), 5.30 (dd, J=12.1, 5.3 Hz, 1H), 4.61-4.56 (m, 1H), 4.44-4.39 (m, 1H), 4.28-4.12 (m, 1H), 4.10-4.06 (m, 1H), 4.02-3.58 (m, 1H), 3.07 (s, 3H), 3.00-2.83 (m, 1H), 2.83-2.73 (m, 1H), 2.70-2.66 (m, 2H), 2.45-2.40 (m, 2H), 2.23-2.00 (m, 6H), 1.97-1.69 (m, 2H), 1.49 (s, 9H); LC/MS (ESI, m/z): [(M+H)]⁺=596.3.

### [(1s,4s)-4-[[1-(2,6-Dioxopiperidin-3-yl)-3-methyl-2-oxo-1,3-benzodiazol-5-yl]methyl]cyclohexyl]acetic acid (Intermediate M)

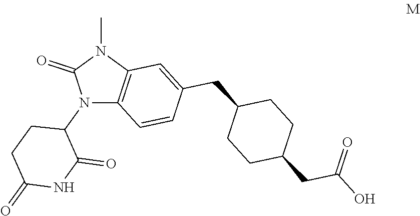 [SMILES: Cn1c(=O)n(C2CCC(=O)NC2=O)c2ccc(C[C@H]3CC[C@@H](CC(=O)O)CC3)cc21]

¹H NMR (300 MHz, DMSO-d₆) δ 12.50 (br, 1H), 11.06 (s, 1H), 6.70-6.96 (m, 2H), 6.82 (dd, J=8.1, 1.5 Hz, 1H), 5.32 (dd, J 12.7, 5.4 Hz, 1H), 3.31 (s, 3H), 3.00-2.79 (m, 1H), 2.78-2.53 (m, 4H), 2.21 (d, J 7.3 Hz, 2H), 2.04-2.00 (m, 1H), 1.91-1.86 (m, 1H), 1.70-1.67 (m, 1H), 1.55-1.48 (m, 6H) 1.35-1.28 (m, 2H). LC/MS (ESI, m/z): [(M+H)]⁺=414.2.

### [(1s,4s)-4-[[3-Methyl-1-(1-methyl-2,6-dioxopiperidin-3-yl)-2-oxo-1,3-benzodiazol-5-yl]methyl]cyclohexyl]acetic acid (Intermediate N)

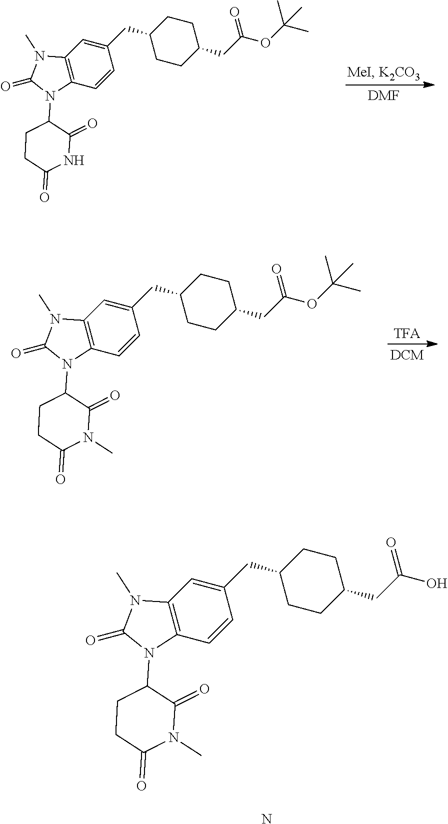

Step 1—tert-butyl 2-[(1s,4s)-4-[[3-methyl-1-(1-methyl-2,6-dioxopiperidin-3-yl)-2-oxo-1,3-benzodiazol-5-yl]methyl]cyclohexyl]acetate. To a stirred mixture of tert-butyl 2-(4-[[1-(2,6-dioxopiperidin-3-yl)-3-methyl-2-oxo-1,3-benzodiazol-5-yl]methylidene]cyclohexyl)acetate (550.00 mg, 1.18 mmol) and MeI (500.89 mg, 3.53 mmol) in DMF (8.00 mL) was added K₂CO₃ (325.14 mg, 2.35 mmol) in portions at rt and the resulting mixture was stirred for overnight. On completion, the residue was purified by silica gel column chromatography, eluted with PE/EtOAc (1:1), to afford the title compound (530 mg, 93% yield) as a light brown solid. ¹H NMR (300 MHz, Chloroform-d) δ 6.89-6.78 (m, 2H), 6.67 (d, J 8.0 Hz, 1H), 5.17 (dd, J 12.5, 5.3 Hz, 1H), 3.43 (s, 3H), 3.25 (s, 3H), 3.10-2.96 (m, 1H), 2.93-2.67 (m, 2H), 2.61 (d, J 7.5 Hz, 2H), 2.28-2.16 (m, 3H), 2.05-2.03 (m, 1H), 1.80-1.68 (m, 1H), 1.55-1.42 (m, 15H), 1.41-1.32 (m, 2H). LC/MS (ESI, m/z): [(M+H)]⁺=484.2.

Step 2—[(1s,4s)-4-[[3-methyl-1-(1-methyl-2,6-dioxopiperidin-3-yl)-2-oxo-1,3-benzodiazol-5-yl]methyl]cyclohexyl]acetic acid. To a stirred solution of tert-butyl 2-[(1s,4s)-4-[[3-methyl-1-(1-methyl-2,6-dioxopiperidin-3-yl)-2-oxo-1,3-benzodiazol-5-yl]methyl]cyclohexyl]acetate (530.00 mg) in TFA (3 mL)) was added DCM (15 mL) dropwise at rt under air atmosphere and the solution was stirred for 2 h. On completion, the reaction mixture was concentrated under reduced pressure to give crude solid. The crude solid was washed with Et₂O (2×30 mL) to afford the title compound (440 mg, 94% yield) as a white solid; ¹H NMR (400 MHz, DMSO-d₆) δ 7.04-6.97 (m, 2H), 6.82 (dd, J 8.2, 1.5 Hz, 1H), 5.43-5.39 (m, 1H), 3.33 (s, 3H), 3.04 (s, 3H), 3.02-2.91 (m, 1H), 2.82-2.63 (m, 2H), 2.58 (d, J 7.5 Hz, 2H), 2.22 (d, J 7.4 Hz, 2H), 2.02-2.00 (m, 1H), 1.92-1.88 (m, 1H), 1.75-1.71 (m, 1H), 1.46-1.40 (m, 6H), 1.34-1.21 (m, 2H); LC/MS (ESI, m/z): [(M+H)]⁺=428.3.

### tert-butyl N-[(5S,8S,10aR)-8-[[(1S)-3-carbamoyl-1-(diphenylmethylcarbamoyl)propyl]carbamoyl]-6-oxo-octahydro-1H-pyrrolo[1,2-a][1,5]diazocin-5-yl]carbamate (Intermediate O)

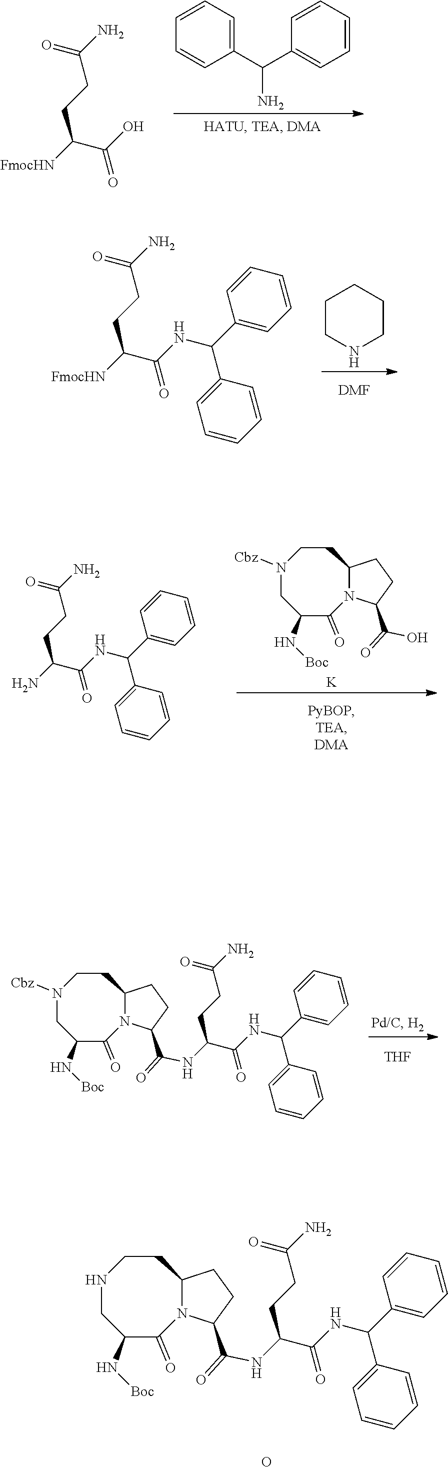

Step 1—9H-fluoren-9-ylmethyl N-[(1S)-3-carbamoyl-1-(diphenylmethylcarbamoyl)propyl]carbamate. To a solution of (2S)-4-carbamoyl-2-[[(9H-fluoren-9-ylmethoxy)carbonyl]amino]butanoic acid (synthesized according to the literature WO2007/1306) (20.0 g, 54.3 mmol) and TEA (11.0 g, 110 mmol) in DMA (400 mL) were added diphenylmethanamine (10.9 g, 0.06 mmol) and HATU (24.8 g, 0.065 mmol) at 25° C. and the mixture was stirred for 16 h. The product was precipitated by the slow addition of water (200 mL) at rt and was collected by filtration and washed with water (2×50.0 mL). The solids were triturated with acetone (100 mL) for 30 min. After filtration, the filtered cake was collected and washed with acetone (2×30.0 mL). The solids were dried under vacuum to afford the title compound as a white solid (38.0 g, 73% yield). ¹H NMR (400 MHz, DMSO-d₆) δ 8.80 (d, J=8.5 Hz, 1H), 7.90 (d, J=7.5 Hz, 2H), 7.74 (dd, J=7.5, 4.8 Hz, 2H), 7.55 (d, J=8.3 Hz, 1H), 7.42 (td, J=7.5, 2.1 Hz, 2H), 7.37-7.20 (m, 12H), 6.78 (s, 1H), 6.11 (d, J=8.4 Hz, 1H), 4.33-4.08 (m, 4H), 2.30-2.03 (m, 2H), 1.91 (ddt, J=15.1, 10.3, 5.2 Hz, 1H), 1.79 (ddt, J=13.6, 9.2, 4.9 Hz, 1H); LC/MS (ESI, m z): [(M+1)]⁺=534.4.

Step 2—(2S)-2-Amino-N-(diphenylmethyl)pentanediamide. To a stirred solution of 9H-fluoren-9-ylmethyl N-[(1S)-3-carbamoyl-1-(diphenylmethylcarbamoyl)propyl]carbamate (4.00 g, 7.50 mmol) in DMF (10.0 mL) was added piperidine (5.00 mL) dropwise at rt under argon atmosphere and the resulting mixture was stirred for 1 h. On completion, the resulting mixture was concentrated under vacuum and the residue was purified by reverse phase flash chromatography (Column: Spherical C¹⁸, 20-40 um, 330 g; Mobile Phase A: water (plus 10 mmol/L NH₄HCO₃); Mobile Phase B: ACN; Flow rate: 80 mL/min; Gradient: 30% B-50% B in 20 min; Detector: UV 254/220 nm; the fractions containing desired product were collected at 39% B) and concentrated under reduced pressure to afford the title compound as an off-white solid (2.0 g, 86% yield): ¹H NMR (400 MHz, DMSO-d₆) δ 8.67 (d, J=8.6 Hz, 1H), 7.39-7.21 (m, 10H), 6.70 (s, 1H), 6.10 (d, J=7.5 Hz, 1H), 3.24 (dd, J=8.3, 5.0 Hz, 1H), 2.23-2.03 (m, 2H), 1.85 (d, J=5.4 Hz, 2H), 1.65-1.51 (m, 1H); LC/MS (ESI, m z): [(M+1)]⁺=312.1.

Step 3—Benzyl (5S,8S,10aR)-5-[(tert-butoxycarbonyl)amino]-8-[[(1S)-3-carbamoyl-1-(diphenylmethylcarbamoyl)propyl]carbamoyl]-6-oxo-octahydropyrrolo[1,2-a][1,5]diazocine-3-carboxylate. To a stirred solution of (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (1.00 g, 2.17 mmol, Intermediate K), (2S)-2-amino-N-(diphenylmethyl)pentanediamide (0.74 g, 2.38 mmol) and TEA (0.44 g, 4.33 mmol) in DMA (20.0 mL) was added PyBOP (1.69 g, 3.25 mmol) in portions at rt under air atmosphere and the mixture was stirred for 16 h. On completion, the reaction was quenched by the addition of water (60.0 mL) and extracted with EtOAc (3×50.0 mL). The combined organic layers were washed with brine (100 mL), and dried over anhydrous Na₂SO₄. After filtration, the filtrate was concentrated under reduced pressure to give the title compound as a yellow oil (1.50 g, crude). ¹H NMR (400 MHz, CDCl₃). 7.41-7.13 (m, 15H), 6.19 (d, J=8.3 Hz, 1H), 5.71-5.45 (m, 2H), 5.17 (s, 2H), 4.64 (dd, J=60.9, 9.1 Hz, 2H), 4.38-4.18 (m, 1H), 3.77-3.50 (m, 2H), 3.35 (s, 1H), 2.50-2.29 (m, 2H), 2.19 (dd, J=26.1, 16.3 Hz, 3H), 2.02-1.89 (m, 3H), 1.73-1.52 (m, 2H), 1.45 (s, 9H); LC/MS (ESI, m z): [(M+1)]⁺=755.3.

Step 4—Tert-butyl N-[(5S,8S,10aR)-8-[[(1S)-3-carbamoyl-1-(diphenylmethylcarbamoyl)propyl]carbamoyl]-6-oxo-octahydro-1H-pyrrolo[1,2-a][1,5]diazocin-5-yl]carbamate. To a solution of benzyl (5S,8S,10aR)-5-[(tert-butoxycarbonyl)amino]-8-[[(1S)-3-carbamoyl-1-(diphenylmethylcarbamoyl)propyl]carbamoyl]-6-oxo-octahydropyrrolo[1,2-a][1,5]diazocine-3-carboxylate (10.0 g, 13.3 mmol) in THF (200 mL) was added 10% palladium on activated carbon (140 mg) under nitrogen atmosphere. The mixture was degassed three times and was hydrogenated at rt for 4 h using a hydrogen balloon. On completion, the mixture was filtered and the filter cake was washed with THF (3×20.0 mL). The combined filtrates were concentrated under reduced pressure. The residue was purified by reverse phase flash chromatography (Column: WelFlash™ C18-I, 20-40 μm, 80 g; Eluent A: water (plus 10 mmol/L TEA); Eluent B: ACN; Gradient: 35%-55% B in 15 min; Flow rate: 50 mL/min; Detector: UV 220/254 nm; desired fractions were collected at 48% B) and concentrated under reduced pressure to afford the title compound as a light yellow solid (7.0 g, 85% yield). ¹H NMR (400 MHz, DMSO-d₆) δ 8.91 (d, J=7.9 Hz, 1H), 8.34 (d, J=8.6 Hz, 1H), 7.40-7.18 (m, 11H), 6.78 (s, 1H), 6.09 (d, J=8.6 Hz, 1H), 5.94 (d, J=7.6 Hz, 1H), 4.70-4.58 (m, 1H), 4.36 (dt, J=19.5, 10.2 Hz, 2H), 4.30-4.17 (m, 1H), 3.13 (d, J=14.1 Hz, 1H), 3.02 (td, J=6.6, 3.9 Hz, 2H), 2.80 (dd, J=11.9, 6.2 Hz, 2H), 2.65 (t, J=12.9 Hz, 1H), 2.47-2.23 (m, 2H), 2.02 (td, J=14.2, 12.5, 6.4 Hz, 2H), 1.91-1.64 (m, 4H), 1.39 (s, 10H); LC/MS (ESI, m z): [(M+1)]⁺=621.3.

### (2S,4R)-4-hydroxy-1-[2-(3-hydroxy-1,2-oxazol-5-yl)-3-methylbutanoyl]-N-[[4-(4-methyl-1,3-thiazol-5-yl)phenyl]methyl]pyrrolidine-2-carboxamide (Intermediate P)

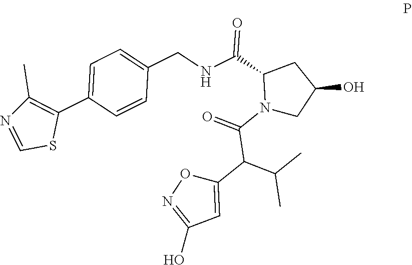 [SMILES: Cc1ncsc1-c1ccc(CNC(=O)[C@@H]2C[C@@H](O)CN2C(=O)C(c2cc(O)no2)C(C)C)cc1]

### (2S,4R)-1-[(2S)-2-(6-[3-[(2S)-2-amino-4-carbamoylbutoxy]-2-fluorophenyl]hexanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate Q)

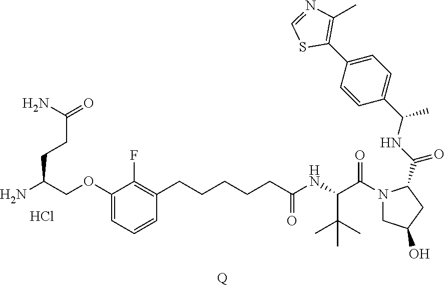

(400 MHz, DMSO-d₆) δ 9.14 (s, 1H), 8.43 (d, J=7.7 Hz, 3H), 7.45 (d, J=8.3 Hz, 4H), 7.39 (d, J=8.3 Hz, 2H), 7.07-7.03 (m, 2H), 6.94 (s, 1H), 6.91-6.87 (m, 1H), 4.94-4.90 (m, 1H), 4.51 (d, J=9.4 Hz, 1H), 4.45-4.41 (m, 1H), 4.30-4.26 (m, 1H), 4.25-4.20 (m, 1H), 4.17-4.15 (m, 2H), 3.68-3.55 (m, 3H), 2.61-2.57 (m, 2H), 2.47 (s, 3H), 2.30-2.26 (m, 2H), 2.08 (s, 1H), 2.17-1.86 (m, 3H), 1.84-1.71 (m, 1H), 1.62-1.42 (m, 5H), 1.40-1.36 (m, 3H), 1.33-1.20 (m, 2H), 0.93 (s, 9H). LC/MS (ESI, m/z): [(M+H)]⁺=768.0.

### (2S,11S)-11-[(tert-butoxycarbonyl)amino]-12-oxo-1-azatricyclo[6.4.1.0[4,13]]trideca-4(13),5,7-triene-2-carboxylic acid (Intermediate R)

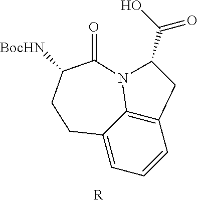

### (2S)-1-[(2S)-2-[(tert-butoxycarbonyl)amino]-3-[1-(triphenylmethyl)imidazol-4-yl]propanoyl]pyrrolidine-2-carboxylic acid (Intermediate S)

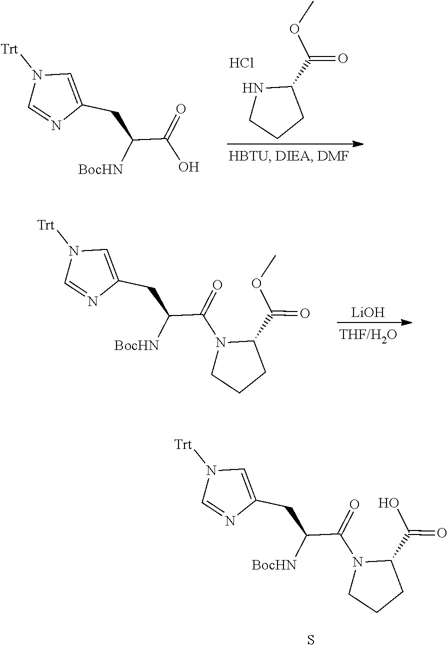

Step 1—Methyl (2S)-1-[(2S)-2-[(tert-butoxycarbonyl)amino]-3-[1-(triphenylmethyl)imidazol-4-yl]propanoyl]pyrrolidine-2-carboxylate. To a stirred solution of (2S)-2-[(tert-butoxycarbonyl)amino]-3-[1-(triphenylmethyl)imidazol-4-yl]propanoic acid (5.00 g, 10.05 mmol, CAS #32926-43-5) and methyl (2S)-pyrrolidine-2-carboxylate (1.56 g, 12.08 mmol) in DMF (50 mL) were added DIEA (5.19 g, 40.19 mmol) and HBTU (4.57 g, 12.06 mmol) in portions at rt under nitrogen atmosphere and the mixture was stirred for 2 h. The resulting mixture was diluted with water (200 mL) and extracted with EtOAc (3×300 mL). The combined organic layers were washed with brine (2×200 mL), and dried over anhydrous Na₂SO₄. After filtration, the filtrate was concentrated under reduced pressure. The crude product was purified by reverse phase flash (Column: Spherical C18 Column, 20-40 um, 330 g; Mobile Phase A: Water (0.1% FA), Mobile Phase B: ACN; Flow rate: 100 mL/min; Gradient: 35% B to 55% B in 25 min, 254 nm; the fractions containing the desired product were collected at 52% B) to afford the title compound (5.5 g, 90% yield) as a brown solid. ¹H NMR (400 MHz, Methanol-d₄) δ 7.73 (s, 1H), 7.45-7.37 (m, 10H), 7.21-7.18 (m, 5H), 7.05 (s, 1H), 4.69-4.62 (m, 1H), 4.52-4.46 (m, 1H), 3.87-3.77 (m, 1H), 3.68-3.63 (m, 1H), 3.57 (s, 3H), 3.05-2.97 (m, 1H), 2.90-2.80 (m, 1H), 2.31-2.21 (m, 1H), 2.04-1.98 (m, 2H), 1.97-1.89 (m, 1H), 1.41 (s, 9H); LC/MS (ESI, m/z): [(M+1)]⁺=609.3.

Step 2—(2S)-1-[(2S)-2-[(tert-butoxycarbonyl)amino]-3-[1-(triphenylmethyl)imidazol-4-yl]propanoyl]pyrrolidine-2-carboxylic acid. To a stirred solution of methyl (2S)-1-[(2S)-2-[(tert-butoxycarbonyl)amino]-3-[1-(triphenylmethyl)imidazol-4-yl]propanoyl]pyrrolidine-2-carboxylate (5.50 g, 9.04 mmol) in THF (25.00 mL) was added a solution of LiOH (1.73 g, 72.28 mmol) in H₂O (25.00 mL) dropwise at rt under nitrogen atmosphere and the mixture was stirred for 2 h. On completion, the mixture was concentrated under reduced pressure. The crude product was purified by reverse phase flash (Column: Spherical C18 Column, 20-40 um, 330 g; Mobile Phase A: Water (0.1% FA), Mobile Phase B: ACN; Flow rate: 100 mL/min; Gradient: 25% B to 50% B in 25 min, 254 nm, the fractions containing the desired product were collected at 34% B) to afford the title compound (4.1 g, 76% yield) as a white solid. ¹H NMR (400 MHz, DMSO-d₆) δ 12.97 (s, 1H), 7.48-7.34 (m, 10H), 7.33-7.31 (m, 1H), 7.11-7.07 (m, 5H), 7.04-6.99 (m, 1H), 6.83-6.78 (m, 1H), 4.40-4.35 (m, 1H), 4.29-4.23 (m, 1H), 3.65-3.53 (m, 1H), 3.43-3.34 (m, 1H), 2.74-2.62 (m, 2H), 2.17-2.10 (m, 1H), 1.92-1.79 (m, 3H), 1.33 (s, 9H); LC/MS (ESI, m z): [(M+1)]⁺=595.4.

### (5S,8S,10aR)-5-[(tert-butoxycarbonyl)amino]-3-(3-fluorobenzoyl)-6-oxo-octahydropyrrolo[1,2-a][1,5]diazocine-8-carboxylic acid (Intermediate T)

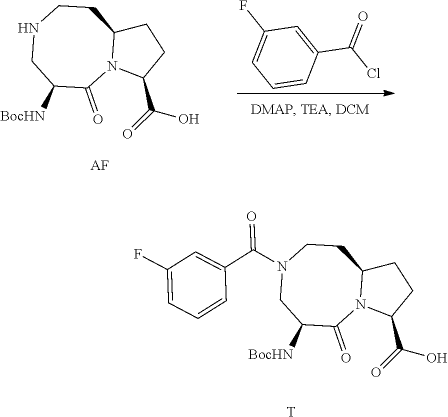

To a stirred mixture of (5S,8S,10aR)-5-[(tert-butoxycarbonyl)amino]-6-oxo-octahydro-1H-pyrrolo[1,2-a][1,5]diazocine-8-carboxylic acid (300.00 mg, 0.92 mmol, Intermediate AF) and TEA (278.18 mg, 2.75 mmol) in DCM (6.00 mL) was added 3-fluoro-benzoyl chloride (290.60 mg, 1.83 mmol) dropwise at 0° C. under nitrogen atmosphere. The resulting mixture was stirred for 3 h at rt. On completion, the reaction mixture was extracted with CH₂Cl₂ (3×10 mL). The combined organic layers were washed with brine (3×10 mL), and dried over anhydrous Na₂SO₄. After filtration, the filtrate was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 60 mL/min; Detector: 220/254 nm; desired fractions were collected at 30% B) and concentrated under reduced pressure to afford the title compound (185 mg, 45% yield) as a white solid. H NMR (300 MHz, Methanol-d₄) δ 8.11 (d, J=6.7 Hz, 1H), 7.48-7.39 (m, 1H), 7.32-7.30 (m, 1H), 7.20-7.14 (m, 1H), 6.90 (d, J=6.7 Hz, 1H), 4.80-4.75 (m, 1H), 4.52-4.25 (m, 3H), 3.97-3.78 (m, 2H), 3.70-3.53 (m, 2H), 3.48-3.43 (m, 1H), 2.40-2.33 (m, 1H), 2.29-1.92 (m, 2H), 1.87-1.66 (m, 1H), 1.40 (s, 9H); LC/MS (ESI, m/z): [(M+H)]⁺=450.2.

### (4-[3-[(2S)-2-[(tert-butoxycarbonyl)amino]-4-carbamoylbutoxy]-2-chlorophenyl]cyclohexyl)acetic acid (Intermediate U)

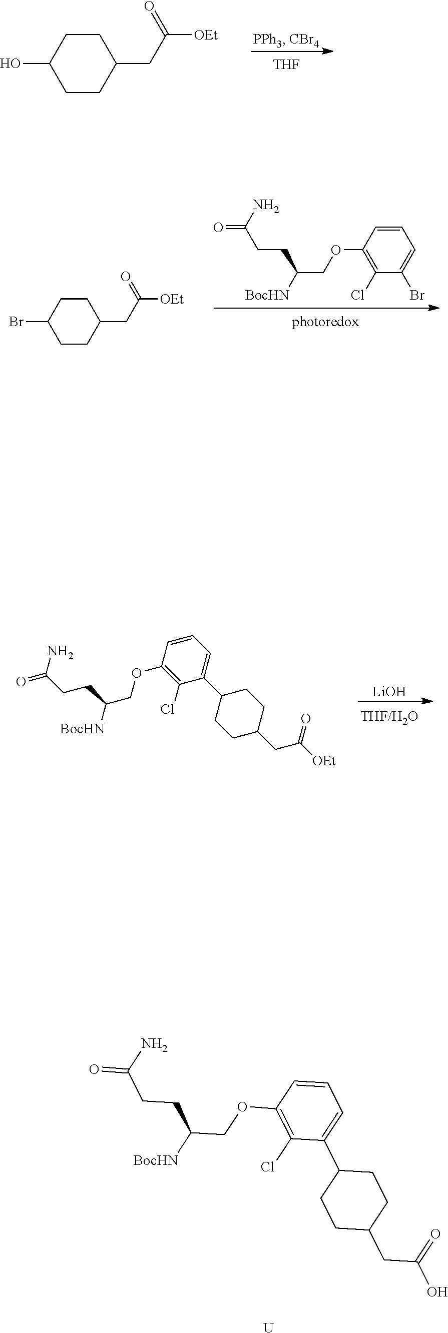

Step 1—Ethyl 2-(4-bromocyclohexyl)acetate. To a stirred mixture of ethyl 2-(4-hydroxycyclohexyl)acetate (2.00 g, 10.74 mmol) and PPh₃ (5.63 g, 21.48 mmol) in THF (20.00 mL) was added CBr₄ (3.92 g, 11.81 mmol) in portions at 0° C. under nitrogen atmosphere. The resulting mixture was stirred for 16 h at rt under nitrogen atmosphere. On completion, the reaction mixture was concentrated under reduced pressure. The residue was purified by silica gel column chromatography, eluted with PE/EtOAc (50:1-20:1), to afford the title compound (2 g, 75% yield) as a colorless oil. ¹H NMR (300 MHz, Chloroform-d) δ 5.73-5.58 (m, 1H), 4.65-4.60 (m, 1H), 4.14 (q, J=7.1 Hz, 2H), 2.28 (d, J=7.1 Hz, 2H), 2.15-2.01 (m, 3H), 1.97-1.67 (m, 2H), 1.64-1.57 (m, 3H), 1.27 (t, J=7.1 Hz, 3H).

Step 2—Ethyl 2-(4-[3-[(2S)-2-[(tert-butoxycarbonyl)amino]-4-carbamoylbutoxy]-2-chlorophenyl]cyclohexyl)acetate. To a stirred mixture of ethyl 2-(4-bromocyclohexyl)acetate (800.00 mg, 3.21 mmol) and tert-butyl N-[(2S)-1-(3-bromo-2-chlorophenoxy)-4-carbamoylbutan-2-yl]carbamate (1489.53 mg, 3.53 mmol, synthesized via Steps 1-3 of Intermediate C) in DME (15.00 mL) were added tris(trimethylsilyl)silane (798.43 mg, 3.21 mmol), IR[DF(CF₃)PPY]₂(DTBPY)PF₆ (36.02 mg, 0.032 mmol) and Na₂CO₃ (1020.97 mg, 9.63 mmol) at rt under nitrogen atmosphere. To the above mixture were added 4-tert-butyl-2-(4-tert-butylpyridin-2-yl)pyridine (8.62 mg, 0.032 mmol) and 1,2-dimethoxyethane dihydrochloride nickel (7.06 mg, 0.032 mmol) in DME (5.00 mL) at rt. The reaction mixture was then irradiated with ultraviolet lamp for 16 h at 25° C. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 35%-60% B in 25 min; Flow rate: 60 mL/min; Detector: 220/254 nm; desired fractions were collected at 55% B) and concentrated under reduced pressure to afford the title compound (310 mg, 19% yield) as a brown solid; ¹H NMR (300 MHz, Chloroform-d) δ 7.20 (t, J=7.9 Hz, 1H), 6.94 (d, J=7.9 Hz, 1H), 6.78 (d, J=8.1 Hz, 1H), 6.54 (s, 1H), 5.68-5.59 (m, 2H), 5.20 (d, J=8.3 Hz, 1H), 4.24-4.11 (m, 2H), 4.07 (s, 3H), 3.08-3.01 (m, 1H), 2.42-2.38 (m, 2H), 2.32-2.26 (m, 2H), 2.17-2.05 (m, 4H), 1.95-1.90 (m, 3H), 1.8-1.70 (m, 1H), 1.48 (s, 9H), 1.33-1.27 (m, 5H); LC/MS (ESI, m z): [(M+H)]⁺=511.3.

Step 3—(4-[3-[(2S)-2-[(tert-butoxycarbonyl)amino]-4-carbamoylbutoxy]-2-chlorophenyl]cyclohexyl)acetic acid. To a stirred mixture of ethyl 2-(4-[3-[(2S)-2-[(tert-butoxycarbonyl)amino]-4-carbamoylbutoxy]-2-chlorophenyl]cyclohexyl)acetate (300.00 mg, 0.59 mmol) in THF (4.00 mL) was added dropwise a solution of LiOH—H₂O (246.34 mg, 5.87 mmol) in H₂O (4.00 mL) at rt under air atmosphere and the mixture was stirred for 16 h. On completion, the reaction mixture was acidified to pH=6 with HCl (aq.). The crude was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 50% B) and concentrated under reduced pressure to afford the title compound (180 mg, 64% yield) as a white solid; ¹H NMR (400 MHz, DMSO-d₆) δ 12.04 (s, 1H), 7.28-7.22 (m, 2H), 6.99-6.95 (m, 2H), 6.81 (d, J=8.4 Hz, 1H), 6.74 (s, 1H), 3.92 (d, J=6.1 Hz, 2H), 3.81-3.74 (m, 1H), 2.96-2.89 (m, 1H), 2.17-2.10 (m, 3H), 1.87-1.72 (m, 6H), 1.69-1.51 (m, 1H), 1.49-1.43 (m, 2H), 1.39 (s, 9H), 1.34-1.30 (m, 1H), 1.21-1.06 (m, 2H); LC/MS (ESI, m/z): [(M+H)]⁺=483.7.

### (2S,4R)-1-[(2S)-2-[2-(4-[3-[(2S)-2-amino-4-carbamoylbutoxy]-2-chlorophenyl]cyclohexyl)acetamido]-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate V)

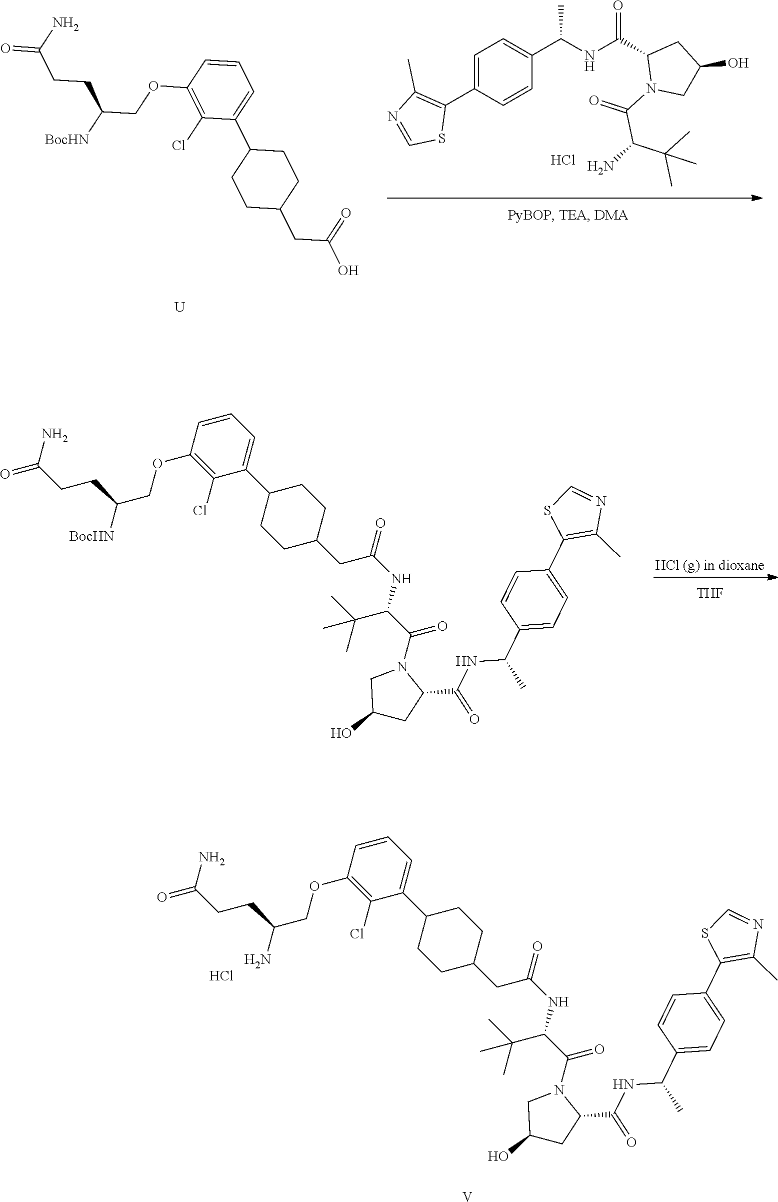

Step 1—tert-butyl N-[(2S)-4-carbamoyl-1-[2-chloro-3-[4-([[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]methyl)cyclohexyl]phenoxy]butan-2-yl]carbamate. To a stirred mixture of (4-[3-[(2S)-2-[(tert-butoxycarbonyl)amino]-4-carbamoylbutoxy]-2-chlorophenyl]cyclohexyl)acetic acid (180.00 mg, 0.37 mmol, Intermediate U) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (198.82 mg, 0.45 mmol, CAS #1448189-80-7) in DMA (4.00 mL) were added PyBOP (290.90 mg, 0.56 mmol) and TEA (113.13 mg, 1.12 mmol,) in turns at rt and the reaction mixture was stirred for 1 h at rt under nitrogen atmosphere. On completion, the residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 51% B) and concentrated under reduced pressure to afford the title compound (190 mg, 56% yield) as a white solid. ¹H NMR (300 MHz, Chloroform-d) δ 8.74 (s, 1H), 7.50 (d, J=7.8 Hz, 1H), 7.46-7.35 (m, 4H), 7.18 (t, J=8.0 Hz, 1H), 6.90 (d, J=7.8 Hz, 1H), 6.76 (dd, J=8.2, 1.3 Hz, 1H), 6.42 (s, 1H), 6.27 (d, J=8.5 Hz, 1H), 5.56-5.52 (m, 1H), 5.19 (d, J=8.5 Hz, 1H), 5.11 (t, J=7.2 Hz, 1H), 4.76 (t, J=7.8 Hz, 1H), 4.59 (d, J=8.7 Hz, 1H), 4.18 (d, J=11.4 Hz, 1H), 4.05 (s, 3H), 3.63 (dd, J=11.4, 3.7 Hz, 1H), 3.06-2.99 (m, 1H), 2.62-2.53 (m, 4H), 2.40-2.36 (m, 2H), 2.28-2.04 (m, 6H), 1.92-1.89 (m, 6H), 1.53-1.38 (m, 15H), 1.28-1.18 (m, 1H), 1.09 (s, 9H); LC/MS (ESI, m z): [(M+H)]⁺=909.4.

Step 2—(2S,4R)-1-[(2S)-2-[2-(4-[3-[(2S)-2-amino-4-carbamoylbutoxy]-2-chlorophenyl]cyclo hexyl)acetamido]-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred mixture of tert-butyl N-[(2S)-4-carbamoyl-1-[2-chl oro-3-[4-([[(2S)-1-[(2S,4R)-4-hydroxy-2-[[(1S)-1-[4-(4-methyl-1,3-thiazol-5-yl)phenyl]ethyl]carbamoyl]p yrrolidin-1-yl]-3,3-dimethyl-1-oxobutan-2-yl]carbamoyl]methyl)cyclohexyl]phenoxy]butan-2-yl]carbama te (190.00 mg, 0.21 mmol) in DCM (4.00 mL) was added HCl (gas) in 1,4-dioxane (1.50 mL) dropwise at rt under air atmosphere and the mixture was stirred for 1 h. On completion, the reaction mixture was con centrated under reduced pressure to give the title compound (200 mg, crude). ¹H NMR (400 MHz, DMSO-d₆) δ 9.06 (s, 1H), 8.42 (d, J=7.8 Hz, 1H), 8.39-8.29 (m, 3H), 7.82 (d, J=9.2 Hz, 1H), 7.49-7.42 (m, 2H), 7.39 (d, J=8.3 Hz, 2H), 7.28 (t, J=8.0 Hz, 1H), 7.04 (d, J=8.1 Hz, 2H), 6.95 (s, 1H), 4.96-4.89 (m, 1 H), 4.54 (d, J=9.4 Hz, 1H), 4.45-4.41 (m, 1H), 4.33-4.19 (m, 2H), 4.16-4.12 (m, 1H), 3.65-3.60 (m, 2H), 2.99-2.85 (m, 1H), 2.47 (s, 3H), 2.36-2.28 (m, 2H), 2.27-1.90 (m, 4H), 1.86-1.69 (m, 6H), 1.50-1.41 (m, 6 H), 1.38 (d, J=7.0 Hz, 3H), 1.16-1.13 (m, 1H), 0.95 (s, 9H); LC/MS (ESI, m z): [(M+H)]⁺=809.4.

### 4-[3-[(2S)-2-[(tert-butoxycarbonyl)amino]-4-carbamoylbutoxy]-2-chloro-5-methylphenyl]butanoic acid (Intermediate W)

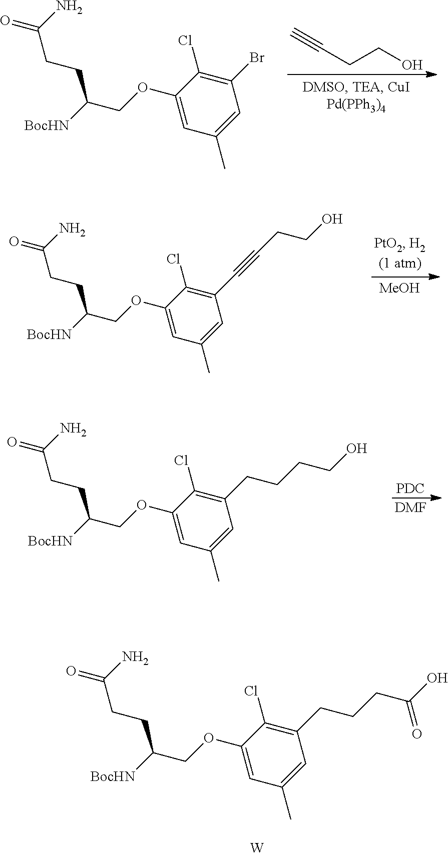

Step 1—Tert-butyl N-[(2S)-4-carbamoyl-1-[2-chloro-3-(4-hydroxybut-1-yn-1-yl)-5-methylphenoxy]butan-2-yl]carbamate. To a stirred mixture of tert-butyl N-[(2S)-1-(3-bromo-2-chloro-5-methylphenoxy)-4-carbamoylbutan-2-yl]carbamate (700.00 mg, 1.61 mmol) and 3-butyn-1-ol (337.80 mg, 4.82 mmol) in DMSO (10.00 mL) were added Pd(PPh₃)₄ (18.56 mg, 0.016 mmol), CuI (3.06 mg, 0.016 mmol) and TEA (6.00 mL, 43.17 mmol) in turns at rt under nitrogen atmosphere. The resulting mixture was stirred for 2 h at 80° C. under nitrogen atmosphere. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 80 mL/min; Detector: 220/254 nm; desired fractions were collected at 50% B) and concentrated under reduced pressure to afford the title compound (400 mg, 59% yield) as a yellow solid. ¹H NMR (300 MHz, Chloroform-d) δ 6.96-6.90 (m, 1H), 6.68 (d, J=1.9 Hz, 1H), 6.32 (s, 1H), 5.46-5.42 (m, 1H), 5.14 (d, J=8.3 Hz, 1H), 4.11-3.95 (m, 3H), 3.85 (t, J=6.1 Hz, 2H), 2.75 (t, J=6.1 Hz, 2H), 2.43-2.32 (m, 2H), 2.31 (s, 3H), 2.12-1.95 (m, 3H), 1.46 (s, 9H); LC/MS (ESI, m/z): [(M+H)]=425.1.

Step 2—tert-butyl N-[(2S)-4-carbamoyl-1-[2-chloro-3-(4-hydroxybutyl)-5-methylphenoxy]butan-2-yl]carbamate. To a solution of tert-butyl N-[(25)-4-carbamoyl-1-[2-chloro-3-(4-hydroxybut-1-yn-1-yl)-5-methylphenoxy]butan-2-yl]carbamate (400.00 mg, 0.94 mmol) in 10 mL THF was added PtO₂ (21.38 mg, 0.094 mmol) under nitrogen atmosphere in a 100 mL round-bottom flask. The mixture was hydrogenated at room temperature for 2 h under hydrogen atmosphere using a hydrogen balloon. On completion, the reaction was filtered through a celite pad and concentrated under reduced pressure to give the title compound (380 mg, 94% yield). ¹H NMR (400 MHz, Chloroform-d) δ 6.71 (s, 1H), 6.63-6.51 (m, 2H), 5.51 (s, 1H), 5.20 (d, J=8.5 Hz, 1H), 4.07-4.03 (m, 3H), 3.70 (t, J=6.1 Hz, 2H), 2.75 (t, J=7.3 Hz, 2H), 2.39 (t, J=6.8 Hz, 2H), 2.31 (s, 3H), 2.17-1.97 (m, 1H), 1.75-1.63 (m, 6H), 1.48 (s, 9H); LC/MS (ESI, m/z): [(M+H)]⁺=429.1.

Step 3—4-[3-[(2S)-2-[(tert-butoxycarbonyl)amino]-4-carbamoylbutoxy]-2-chloro-5-methylphenyl]butanoic acid. To a stirred mixture of tert-butyl N-[(2S)-4-carbamoyl-1-[2-chloro-3-(4-hydroxybutyl)-5-methylphenoxy]butan-2-yl]carbamate (380.00 mg, 0.89 mmol) in DMF (8.00 mL) was added PDC (1666.36 mg, 4.43 mmol) at rt and the mixture was stirred for 16 h under nitrogen atmosphere. On completion, the residue was purified by reverse phase flash chromatography (Column: WelFlash™ C18-I, 20-40 um, 120 g; Eluent A: Water (plus 10 mmol/L FA); Eluent B: ACN; Gradient: 25%-50% B in 25 min; Flow rate: 60 mL/min; Detector: 220/254 nm; desired fractions were collected at 46% B) and concentrated under reduced pressure to the title compound (252 mg, 64% yield) as a yellow solid. ¹H NMR (300 MHz, DMSO-d₆) δ 12.04 (s, 1H), 7.25 (s, 1H), 6.91-6.62 (m, 3H), 3.90-3.88 (m, 2H), 3.81-3.69 (m, 1H), 2.64 (t, J=8.8 Hz, 2H), 2.26-2.20 (m, 5H), 2.16-2.09 (m, 2H), 1.89-1.73 (m, 4H), 1.67-1.54 (m, 1H), 1.38 (s, 9H); LC/MS (ESI, m/z): [(M+H)]⁺=443.2.

### (2S,4R)-1-[(2S)-2-(4-[3-[(2S)-2-amino-4-carbamoylbutoxy]-2-chloro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate X)

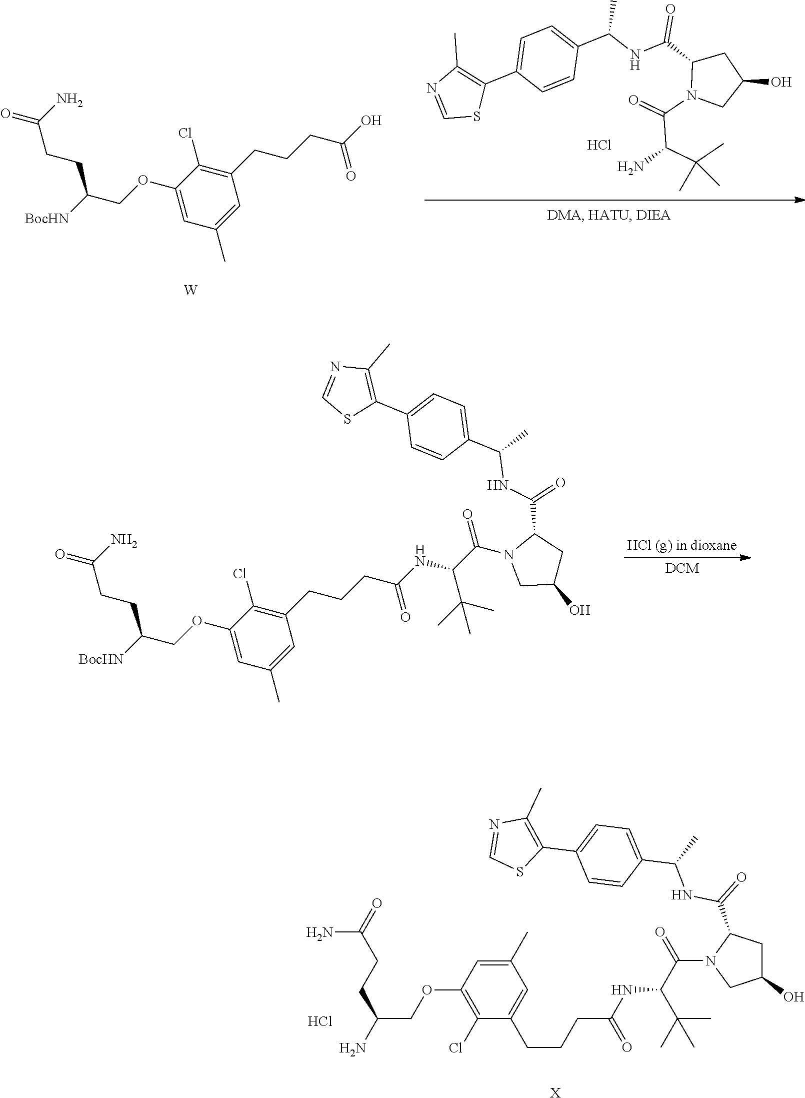

Step 1—tert-butyl N-[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate. To a stirred mixture of 4-[3-[(2S)-2-[(tert-butoxycarbonyl)amino]-4-carbamoylbutoxy]-2-chloro-5-methylphenyl]butanoic acid (250.00 mg, 0.56 mmol, Intermediate W) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (325.81 mg, 0.68 mmol, CAS #1448189-80-7) in DMA (5.00 mL) were added HATU (321.91 mg, 0.85 mmol) and TEA (171.34 mg, 1.69 mmol) at rt under nitrogen atmosphere and the mixture was stirred for 1 h. On completion, the residue was purified by reverse phase flash chromatography (Column: WelFlash™ C18-I, 20-40 um, 120 g; EluentA: Water (plus 10 mmol/L FA); Eluent B: ACN; Gradient: 25%-50% B in 25 min; Flow rate: 60 mL/min; Detector: 220/254 nm; desired fractions were collected at 49% B) and concentrated under reduced pressure to afford the title compound (410 mg, 84% yield) as a white solid. ¹H NMR (300 MHz, Chloroform-d) δ 8.69 (s, 1H), 7.49 (d, J=7.8 Hz, 1H), 7.44-7.33 (m, 4H), 6.65-6.58 (m, 2H), 6.45-6.27 (m, 2H), 5.73 (s, 1H), 5.18 (d, J=8.3 Hz, 1H), 5.13-5.04 (m, 1H), 4.72 (t, J=7.8 Hz, 1H), 4.59 (d, J=8.7 Hz, 1H), 4.52 (s, 1H), 4.11 (dd, J=11.0, 8.7 Hz, 1H), 4.05-3.99 (m, 3H), 3.64-3.60 (m, 1H), 2.79-2.64 (m, 2H), 2.53 (s, 3H), 2.51-2.45 (m, 1H), 2.37-2.32 (m, 2H), 2.27 (s, 3H), 2.24-2.21 (m, 1H), 2.13-1.99 (m, 4H), 1.98-1.90 (m, 3H), 1.49-1.45 (m, 12H), 1.05 (s, 9H); LC/MS (ESI, m z): [(M+H)]⁺=869.3.

Step 2—(2S,4R)-1-[(2S)-2-(4-[3-[(2S)-2-amino-4-carbamoylbutoxy]-2-chloro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred mixture of tert-butyl N-[(2S)-4-carbamoyl-1-[2-chloro-3-(3-[[(2S)-1-[(2S,4R)-4-hydroxy-2-[[(1S)-1-[4-(4- methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate (4 00.00 mg, 0.46 mmol) in DCM (8.00 mL) was added HCl (gas) in 1,4-dioxane (3.00 mL) dropwise at rt under air atmosphere and mixture was stirred for 1 h. On completion, the mixture was concentrated under reduced pressure to give the title compound (390 mg, crude). ¹H NMR (300 MHz, DMSO-d₆) δ 9.06 (s, 1H), 8.38-8.36 (m, 5H), 7.84 (d, J=9.2 Hz, 1H), 7.44-7.38 (m, 5H), 6.95-6.71 (m, 2H), 4.96-4.86 (m, 1H), 4.52 (d, J=9.3 Hz, 1H), 4.42 (t, J=8.0 Hz, 1H), 4.31-4.17 (m, 2H), 4.15-4.10 (m, 1H), 3.62-3.59 (m, 3H), 2.65-2.61 (m, 2H), 2.46 (s, 3H), 2.34-2.25 (m, 6H), 2.22-2.12 (m, 1H), 2.04-1.90 (m, 3H), 1.83-1.72 (m, 3 H), 1.37 (d, J=7.0 Hz, 3H), 0.94 (s, 9H); LC/MS (ESI, m z): [(M+H)]⁺=769.3.

### tert-butyl N-[(2S)-1-(3-bromo-2-fluoro-5-methylphenoxy)-4-carbamoylbutan-2-yl]carbamate (Intermediate Y)

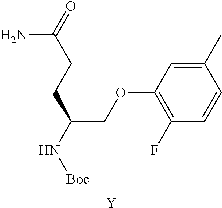

¹H NMR (400 MHz, DMSO-d₆) δ 7.27 (s, 1H), 7.04 (d, J=6.4 Hz, 2H), 6.85 (d, J=8.4 Hz, 1H), 6.75 (s, 1H), 4.01-3.90 (m, 2H), 3.78-3.69 (m, 1H), 2.27 (s, 3H), 2.22-2.06 (m, 2H), 1.87-1.72 (m, 2H), 1.38 (s, 9H). LC/MS (ESI, m z): [(M+H)]⁺=419.1, 421.1.

### 4-[3-[(2S)-2-[(tert-butoxycarbonyl)amino]-4-carbamoylbutoxy]-2-fluoro-5-methylphenyl]butanoic acid (Intermediate Z)

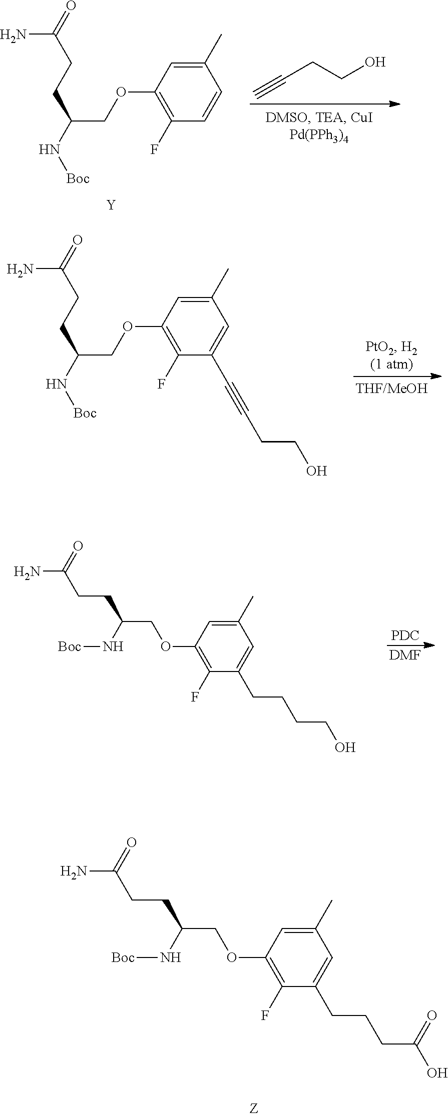

Step 1—Tert-butyl N-[(2S)-4-carbamoyl-1-[2-fluoro-3-(4-hydroxybutyl)-5-methylphenoxy]butan-2-yl]carbamate. To a stirred solution of tert-butyl N-[(2S)-1-(3-bromo-2-fluoro-5-methylphenoxy)-4-carbamoylbutan-2-yl] carbamate (800.00 mg, 1.91 mmol, Intermediate Y) and 3-butyn-1-ol (401.20 mg, 5.72 mmol) in DMSO (20.00 mL) were added TEA (10.00 mL, 71.94 mmol), Pd(PPh₃)₄ (220.48 mg, 0.19 mmol) and CuI (36.34 mg, 0.11 mmol) at rt under nitrogen atmosphere. The resulting mixture was stirred for 2 h at 80° C. under nitrogen atmosphere. On completion, the mixture was concentrated under reduced pressure. The reaction liquid was purified by reverse phase flash (Column: Spherical C18 Column, 20-40 um, 330 g; Mobile Phase A: Water (10 mmOl/L NH₄HCO₃), Mobile Phase B: ACN; Flow rate: 60 mL/min; Gradient: 30% B to 50% B in 25 min, 254 nm; the fractions containing the desired product were collected at 40% B) to afford the title compound (670 mg, 86% yield) as a white solid. ¹H NMR (300 MHz, Methanol-d₄) δ 6.90 (d, J=7.6 Hz, 1H), 6.80 (d, J=5.5 Hz, 1H), 4.03-3.95 (m, 2H), 3.93-3.82 (m, 1H), 3.80-3.69 (m, 2H), 2.71-2.61 (m, 2H), 2.40-2.30 (m, 2H), 2.27 (s, 3H), 2.11-1.93 (m, 1H), 1.89-1.70 (m, 1H), 1.46 (s, 9H); LC/MS (ESI, m/z): [(M+H)]⁺=409.2.

Step 2—Tert-butyl N-[(2S)-4-carbamoyl-1-[2-fluoro-3-(4-hydroxybutyl)-5-methylphenoxy]butan-2-yl]carbamate. To a stirred solution of tert-butyl N-[(2S)-4-carbamoyl-1-[2-fluoro-3-(4-hydroxybut-1-yn-1-yl)-5-methylphenoxy]butan-2-yl]carbamate (670.00 mg, 1.64 mmol) in THF (15.00 mL) was added PtO₂ (100.57 mg, 0.44 mmol) in portions at rt under nitrogen atmosphere and the mixture was stirred for 3 h under hydrogen atmosphere. On completion, the resulting mixture was filtered and the filter cake was washed with MeOH (3×30 mL). The filtrate was concentrated under reduced pressure and the crude product was purified by reverse phase flash (Column: Spherical C18 Column, 20-40 um, 330 g; Mobile Phase A: Water (10 mmol/L, NH₄HCO₃), Mobile Phase B: ACN; Flow rate: 80 mL/min; Gradient: 30% B to 50% B in 25 min, 254 nm; the fractions containing the desired product were collected at 38% B) to afford the title compound (380 mg, 56% yield) as a white solid. ¹H NMR (300 MHz, Methanol-d₄) δ 6.78-6.73 (m, 1H), 6.66-6.60 (m, 1H), 4.01-3.95 (m, 2H), 3.92-3.83 (m, 1H), 3.61-3.52 (m, 2H), 2.68-2.56 (m, 2H), 2.39-2.30 (m, 2H), 2.27 (s, 3H), 2.09-1.96 (m, 1H), 1.89-1.75 (m, 1H), 1.69-1.53 (m, 4H), 1.46 (s, 9H). LC/MS (ESI, m/z): [(M+H)]⁺=413.2.

Step 3—4-[3-[(2S)-2-[(tert-butoxycarbonyl)amino]-4-carbamoylbutoxy]-2-fluoro-5-methylphenyl]butanoic acid. To a stirred solution of tert-butyl N-[(2S)-4-carbamoyl-1-[2-fluoro-3-(4-hydroxybutyl)-5-methylphenoxy]butan-2-yl]carbamate (380.00 mg, 0.92 mmol) in DMF (10.00 mL) was added PDC (1732.81 mg, 4.61 mmol) in portions rt under nitrogen atmosphere and the mixture was stirred for 2 h. On completion, the reaction mixture was purified by reverse phase flash (Column: Spherical C18 Column, 20-40 um, 120 g; Mobile Phase A: Water (0.1% FA), Mobile Phase B: ACN; Flow rate: 60 mL/min; Gradient: 40% B to 60% B in 25 min, 254 nm; the fractions containing the desired product were collected at 48% B) to afford the title compound (330 mg, 84% yield) as a white solid. ¹H NMR (300 MHz, Methanol-d₄) δ 6.80-6.72 (m, 1H), 6.64-6.60 (m, 1H), 3.99-3.93 (m, 2H), 3.92-3.82 (m, 1H), 2.70-2.58 (m, 2H), 2.40-2.29 (m, 4H), 2.27 (s, 3H), 2.06-1.97 (m, 1H), 1.93-1.75 (m, 3H), 1.45 (s, 9H); LC/MS (ESI, m/z): [(M+H)]⁺=427.2.

### (2S,4R)-1-[(2S)-2-(4-[3-[(2S)-2-amino-4-carbamoylbutoxy]-2-fluoro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate AA)

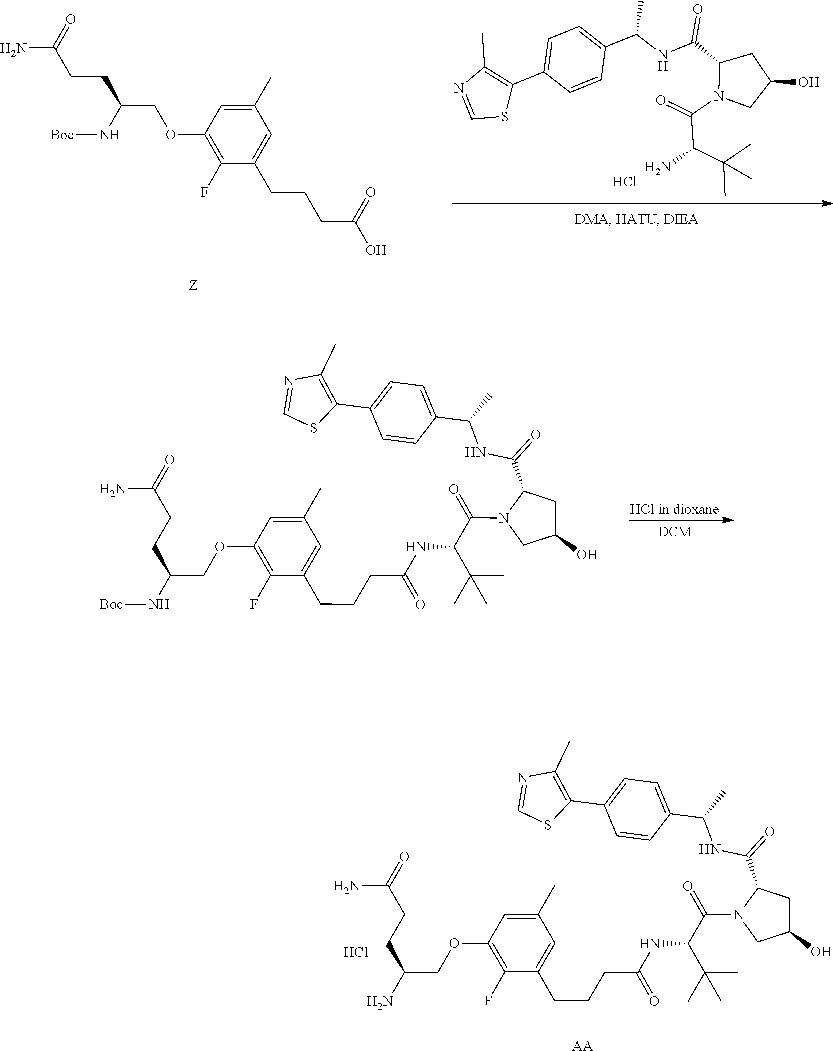

Step 1—Tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate. To a stirred solution of 4-[3-[(2S)-2-[(tert-butoxycarbonyl)amino]-4-carbamoylbutoxy]-2-fluoro-5-methylphenyl]butanoic acid (330.00 mg, 0.774 mmol, Intermediate Z) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (483.89 mg, 1.01 mmol, CAS #1448189-80-7) in DMA (10.00 mL) were added PyBOP (523.46 mg, 1.01 mmol) and TEA (313.19 mg, 3.10 mmol) dropwise at rt under nitrogen atmosphere and the mixture was stirred for 2 h. On completion, the reaction liquid was purified by reverse phase flash (Column: Spherical C18 Column, 20-40 um, 120 g; Mobile Phase A: Water (10 mmol/L NH₄HCO₃), Mobile Phase B: ACN; Flow rate: 60 mL/min; Gradient: 30% B to 50% B in 25 min, 254 nm; the fractions containing the desired product were collected at 45% B) to afford the title compound (420 mg, 64% yield) as a white solid. ¹H NMR (300 MHz, Methanol-d₄) δ 8.92-8.87 (m, 1H), 7.49-7.40 (m, 4H), 6.82-6.73 (m, 1H), 6.68-6.61 (m, 1H), 5.06-4.96 (m, 1H), 4.67-4.54 (m, 2H), 4.48-4.33 (m, 1H), 4.01-3.85 (m, 4H), 3.82-3.71 (m, 1H), 2.69-2.58 (m, 2H), 2.52-2.47 (m, 3H), 2.39-2.16 (m, 8H), 2.09-1.71 (m, 6H), 1.56-1.50 (m, 3H), 1.46 (s, 9H), 1.07 (s, 9H); LC/MS (ESI, m/z): [(M+H)]⁺=853.4.

Step 2—(2S,4R)-1-[(2S)-2-(4-[3-[(2S)-2-amino-4-carbamoylbutoxy]-2-fluoro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate (420.00 mg, 0.49 mmol) in DCM (10.00 mL) was added 4 M HCl (gas) in 1,4-dioxane (5.00 mL) dropwise at rt under nitrogen atmosphere and the mixture was stirred for 1 h. On completion, the mixture was concentrated under reduced pressure to give the title compound. ¹H NMR (300 MHz, DMSO-d₆) δ 9.08-9.03 (m, 1H), 8.42-8.29 (m, 3H), 7.89-7.82 (m, 1H), 7.49-7.37 (m, 5H), 6.98-6.84 (m, 2H), 6.72-6.65 (m, 1H), 4.98-4.87 (m, 1H), 4.57-4.50 (m, 1H), 4.48-4.38 (m, 2H), 4.20-4.18 (m, 2H), 4.17-4.09 (m, 3H), 3.67-3.60 (m, 2H), 2.47 (s, 3H), 2.35-2.11 (m, 8H), 2.08-1.98 (m, 1H), 1.96-1.87 (m, 2H), 1.85-1.70 (m, 3H), 1.38 (d, J=6.9 Hz, 3H), 0.95 (s, 9H); LC/MS (ESI, m/z): [(M+H)]⁺=753.4.

### (5S,8S,10aR)-5-[(tert-butoxycarbonyl)amino]-3-methyl-6-oxo-octahydropyrrolo[1,2-a][1,5]diazocine-8-carboxylic acid (Intermediate AB)

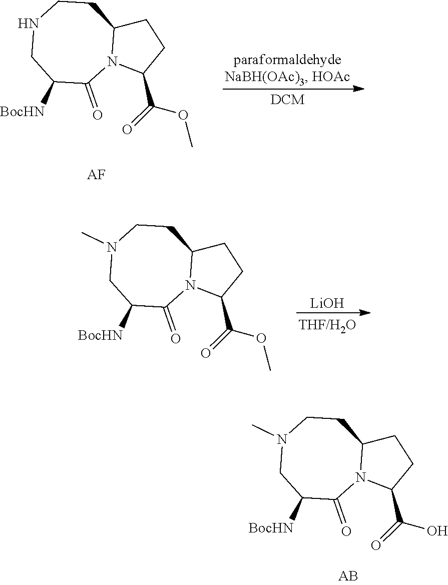

Step 1—Methyl (5S,8S,10aR)-5-[(tert-butoxycarbonyl)amino]-3-methyl-6-oxo-octahydropyrrolo[1,2-a][1,5]diazocine-8-carboxylate. To a stirred solution of methyl (5S,8S,10aR)-5-[(tert-butoxycarbonyl)amino]-6-oxo-octahydro-1H-pyrrolo[1,2-a][1,5]diazocine-8-carboxylate (50.00 mg, 0.15 mmol, Intermediate AF), paraformaldehyde (26.28 mg, 0.29 mmol) and HOAc (8.79 mg, 0.15 mmol) in DCM (3.00 mL) was NaBH(OAc)₃ (62.08 mg, 0.29 mmol) in portions at rt under nitrogen atmosphere and the mixture was stirred for 1 h. On completion, the reaction mixture was quenched with saturated aq. NH₄Cl (10 mL) and the resulting mixture was extracted with DCM (3×20 mL). The combined organic layers were washed with brine (20 mL) and dried over anhydrous Na₂SO₄. After filtration, the filtrate was concentrated under reduced pressure to give the title compound (305.00 mg, crude) as a yellow solid. ¹H NMR (300 MHz, Chloroform-d) δ 5.68 (d, J=7.3 Hz, 1H), 4.62-4.50 (m, 1H), 4.48-4.44 (m, 1H), 4.39-4.29 (m, 1H), 3.77 (s, 3H), 3.03-3.00 (m, 1H), 2.89-2.85 (m, 2H), 2.77-2.73 (m, 1H), 2.55 (s, 3H), 2.43-2.27 (m, 1H), 2.23-1.92 (m, 2H), 1.90-1.73 (m, 2H), 1.70-1.53 (m, 1H), 1.44 (s, 9H); LC/MS (ESI, m z): [(M+1)]⁺=356.2.

Step 2—(5S,8S,10aR)-5-[(tert-butoxycarbonyl)amino]-3-methyl-6-oxo-octahydropyrrolo[1,2-a][1,5]diazocine-8-carboxylic acid. To a stirred solution of methyl (5S,8S,10aR)-5-[(tert-butoxycarbonyl)amino]-3-methyl-6-oxo-octahydropyrrolo[1,2-a][1,5]diazocine-8-carboxylate (305.00 mg, 0.86 mmol) in THF (5.00 mL) was added a solution of LiOH (205.50 mg, 8.58 mmol) in H₂O (5.00 mL) dropwise at rt under air atmosphere and the mixture was stirred for 3 h. The residue was purified by reverse flash chromatography (column, C¹⁸ silica gel; mobile phase, MeCN in water (10 mmol/L FA), 30% to 50% gradient in 25 min; detector, UV 220 nm) to afford the title compound (289 mg, 99% yield) as a white solid. ¹H NMR (400 MHz, Chloroform-d) δ 5.94 (d, J=8.0 Hz, 1H), 5.00-4.97 (m, 1H), 4.82-4.79 (m, 1H), 4.42-4.38 (m, 1H), 3.36-3.25 (m, 1H), 3.18-3.08 (m, 2H), 3.03-2.94 (m, 1H), 2.71 (s, 3H), 2.62-2.52 (m, 1H), 2.31-2.19 (m, 1H), 2.18-2.10 (m, 2H), 1.85-1.79 (m, 1H), 1.77-1.69 (m, 1H), 1.46 (s, 9H); LC/MS (ESI, m z): [(M+1)]⁺=342.2.

### (2S,4S)-1-[(2S)-2-(5-[3-[(2S)-2-amino-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate AC)

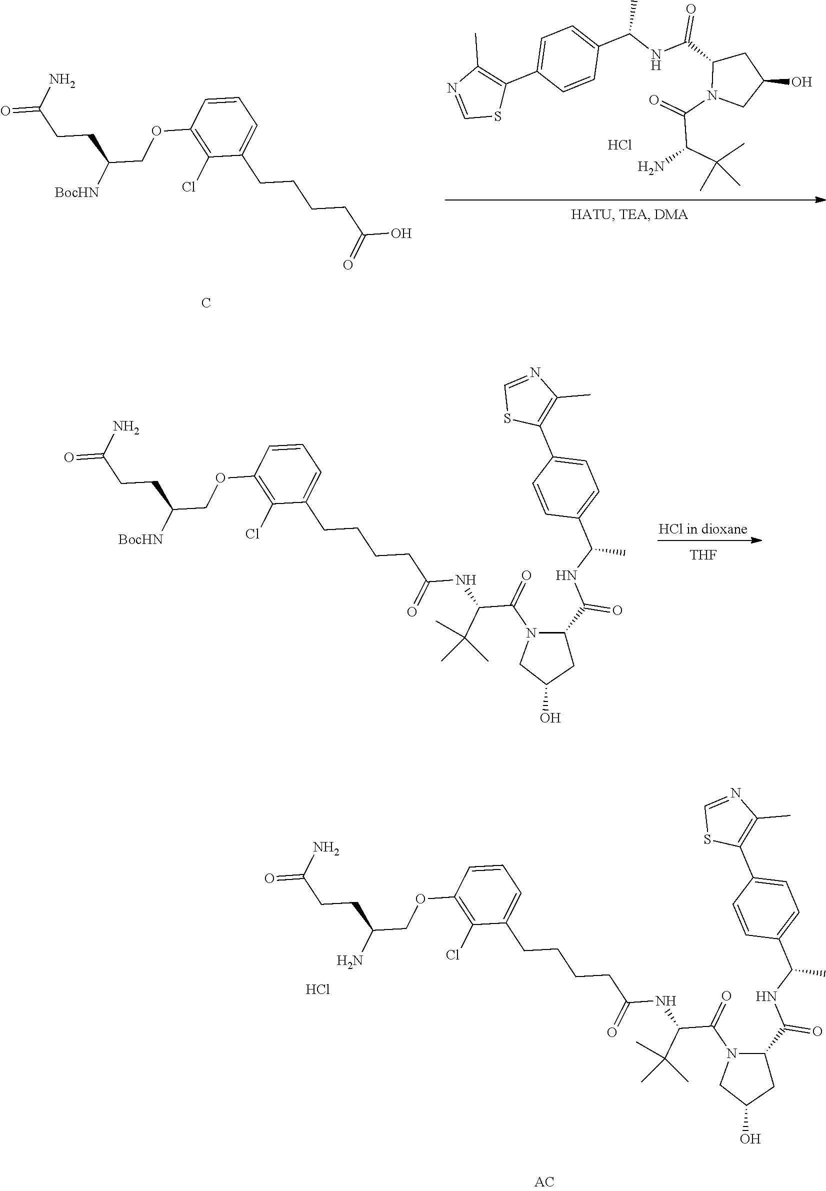

Step 1—Tert-butyl N-[(2S)-4-carbamoyl-1-[2-chloro-3-(4-[[(2S)-1-[(2S,4S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate. To a stirred solution of 5-[3-[(2S)-2-[(tert-butoxycarbonyl)amino]-4-carbamoylbutoxy]-2-chlorophenyl]pentanoic acid (500.00 mg, 1.13 mmol, Intermediate C) and TEA (342.68 mg, 3.39 mmol) in DMA (5.00 mL) were added HATU (515.05 mg, 1.36 mmol) and (2S,4S)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (651.62 mg, 1.36 mmol, CAS #2380273-26-5) in portions at rt under nitrogen atmosphere and the reaction ws stirred for 2 h. On completion, the residue was purified by reverse flash chromatography (column, C18 silica gel; mobile phase, MeCN in water (10 mmol/L FA), 40% to 60% gradient in 20 min; detector, UV 220 nm) to afford the title compound (853 mg, 87% yield) as a white solid. ¹H NMR (300 MHz, Chloroform-d) δ 8.71 (s, 1H), 7.65 (d, J=7.8 Hz, 1H), 7.40 (q, J=8.2 Hz, 4H), 7.13-7.09 (m, 1H), 6.89-6.80 (m, 1H), 6.76-6.74 (m, 1H), 6.34 (s, 1H), 6.10 (d, J=9.0 Hz, 1H), 5.62-5.55 (m, 1H), 5.46-5.38 (m, 1H), 5.19-5.03 (m, 2H), 4.72 (d, J=8.9 Hz, 1H), 4.59 (d, J=9.0 Hz, 1H), 4.48-4.44 (m, 1H), 4.05-4.02 (m, 2H), 3.96-3.92 (m, 1H), 3.85-3.81 (m, 1H), 2.75 (t, J=7.0 Hz, 2H), 2.55 (s, 3H), 2.41-2.21 (m, 4H), 2.19-1.96 (m, 3H), 1.77-1.59 (m, 4H), 1.50 (d, J=6.9 Hz, 3H), 1.45 (s, 9H), 1.32-1.27 (m, 2H), 1.05 (s, 9H); LC/MS (ESI, m z): [(M+1)]⁺=869.5.

Step 2—(2S,4S)-1-[(2S)-2-(5-[3-[(2S)-2-amino-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-chloro-3-(4-[[(2S)-1-[(2S,4S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate (853.00 mg, 0.98 mmol) in THF (5 mL) was added HCl (gas) in 1,4-dioxane (5 mL) dropwise at rt under nitrogen atmosphere and the mixture was stirred for 1 h. On completion, the resulting mixture was concentrated under reduced pressure to afford the title compound (767 mg, 97% yield) as a light yellow solid. ¹H NMR (300 MHz, DMSO-d₆) δ 9.15 (d, J=2.3 Hz, 1H), 8.46 (s, 2H), 8.39 (d, J=7.4 Hz, 1H), 7.87 (d, J=8.7 Hz, 1H), 7.51-7.38 (m, 4H), 7.26-7.06 (m, 2H), 7.01-6.94 (m, 1H), 5.00-4.88 (m, 1H), 4.46 (d, J=8.7 Hz, 1H), 4.39-4.31 (m, 1H), 4.28-4.16 (m, 3H), 3.98-3.82 (m, 1H), 3.68-3.59 (m, 2H), 3.41-3.38 (m, 1H), 2.73-2.70 (m, 3H), 2.49 (s, 3H), 2.40-2.26 (m, 4H), 2.20-1.98 (m, 3H), 1.71-1.57 (m, 1H), 1.59-1.46 (m, 5H), 1.40 (d, J=6.8 Hz, 3H), 0.96 (s, 9H); LC/MS (ESI, m/z): [(M+1)]⁺=769.5.

### 2S,4S)-1-[(2S)-2-(4-[3-[(2S)-2-amino-4-carbamoylbutoxy]-2-chlorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (Intermediate AD)

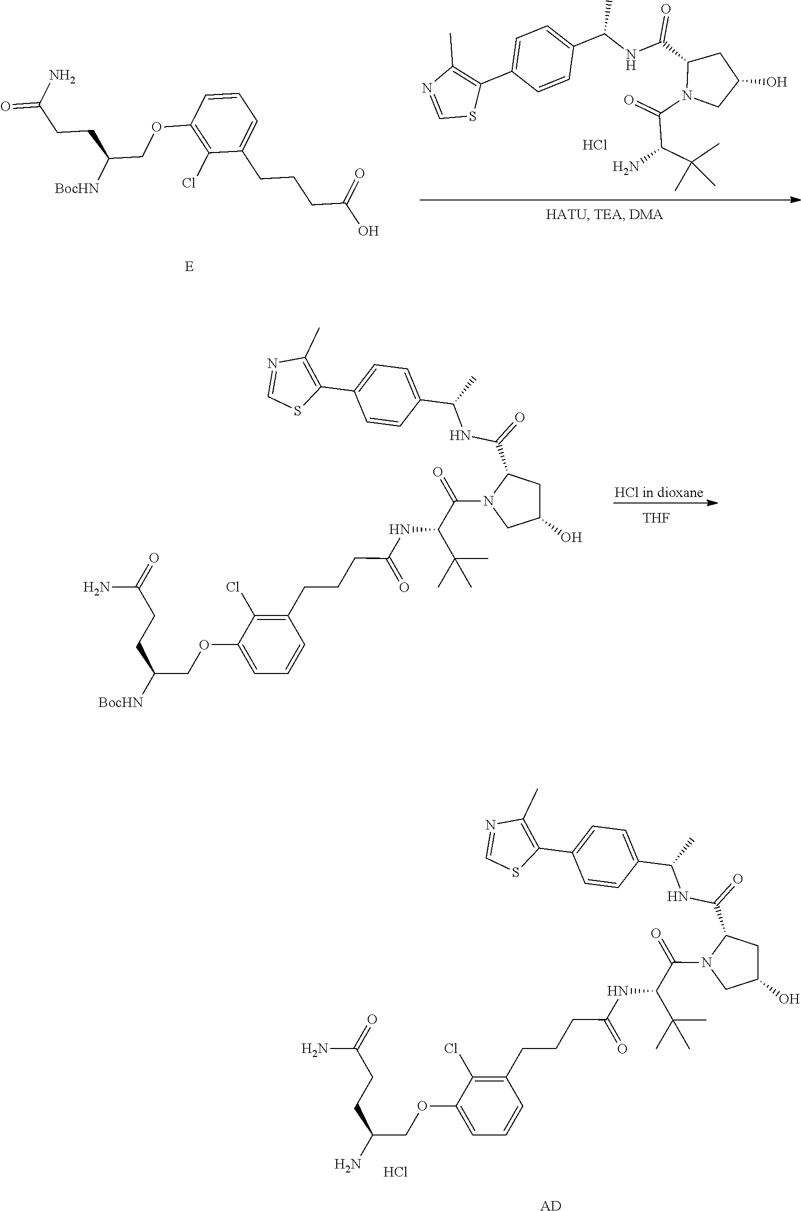

Step 1—Tert-butyl N-[(2S)-4-carbamoyl-1-[2-chloro-3-(3-[[(2S)-1-[(2S,4S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate. To a stirred solution of 4-[3-[(2S)-2-[(tert-butoxycarbonyl)amino]-4-carbamoylbutoxy]-2-chlorophenyl]butanoic acid (500.00 mg, 1.17 mmol, Intermediate E) an d TEA (353.89 mg, 3.50 mmol) in DMA (5 mL) were added HATU (531.90 mg, 1.40 mmol) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (672.94 mg, 1.40 mmol, CAS #2380273-26-5) in portions at rt under nitrogen atmosphere and the mixture was stirred for 2 h. On completion, the residue was purified by reverse flash chromatography (Column: WelFlash™ C18-I, 20-40 μm, 330 g; mobile phase B: MeCN; mobile phase A: water (10 mmol/L FA), 40% to 55% gradient in 20 min; detector: UV 220 nm) to afford the title compound (851 mg, 85% yield) as a white solid. ¹H NMR (300 MHz, Chloroform-d) δ 8.74 (s, 1H), 7.71 (d, J=8.1 Hz, 1H), 7.42 (q, J=8.2 Hz, 4H), 7.15 (t, J=7.9 Hz, 1H), 6.88 (d, J=7.6 Hz, 1H), 6.83-6.76 (m, 1H), 6.33 (s, 1H), 6.20 (d, J=9.1 Hz, 1H), 5.66 (s, 1H), 5.49-5.43 (m, 1H), 5.19-4.99 (m, 1H), 4.7 5 (d, J=8.8 Hz, 1H), 4.63 (d, J=9.0 Hz, 1H), 4.50-4.46 (m, 1H), 4.07-4.38 (m, 3H), 4.02-3.91 (m, 1H), 3.91-3.82 (m, 1H), 2.89-2.74 (m, 2H), 2.56 (s, 3H), 2.46-2.24 (m, 5H), 2.22-1.88 (m, 6H), 1.52 (d, J=6.9 Hz, 3H), 1.47 (s, 9H), 1.08 (s, 9H); LC/MS (ESI, m z): [(M+1)]⁺=855.5.

Step 2—(2S,4S)-1-[(2S)-2-(4-[3-[(2S)-2-amino-4-carbamoylbutoxy]-2-chlorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-chloro-3-(3-[[(2S)-1-[(2S,4S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate (851.00 mg, 1.00 mmol) in THF (5 mL) was added HCl (gas) in 1,4-dioxane (5 mL) dropwise at rt under nitrogen atmosphere and the mixture was stirred for 1 h. On completion, the resulting mixture was concentrated under reduced pressure to afford the title compound (769 mg, 98% yield) as a light yellow solid. ¹H NMR (300 MHz, DMSO-d₆) δ 9.20 (s, 1H), 8.47 (s, 3H), 8.39 (d, J=7.7 Hz, 1H), 7.90 (d, J=8.7 Hz, 1H), 7.51-7.35 (m, 4H), 7.25 (t, J=7.9 Hz, 1H), 7.06 (d, J=8.2 Hz, 1H), 6.95 (d, J=7.4 Hz, 1H), 4.97-4.93 (m, 1H), 4.46 (d, J=8.6 Hz, 1H), 4.42-4.33 (m, 1H), 4.32-4.10 (m, 3H), 3.94-3.90 (m, 1H), 3.65-3.59 (m, 2H), 3.42-3.36 (m, 1H), 2.71-2.67 (m, 2H), 2.48 (s, 3H), 2.37-2.14 (m, 4H), 2.00-1.94 (m, 2H), 180-1.73 (m, 6H), 1.38 (d, J=6.9 Hz, 3H), 0.96 (s, 9H); LC/MS (ESI, m z): [(M+1)]⁺=755.5.

### (2S)-2-Amino-N-[(2-chloro-4-isopropylphenyl)methyl]pentanediamide hydrochloride (Intermediate AE)

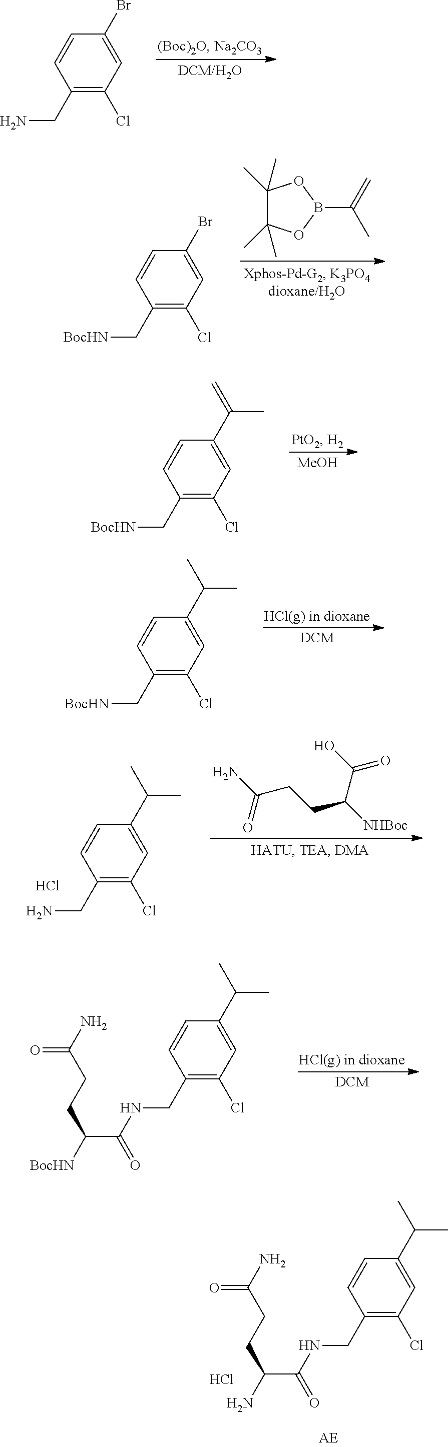

Step 1—Tert-butyl N-[(4-bromo-2-chlorophenyl)methyl]carbamate. To a stirred solution of 1-(4-bromo-2-chlorophenyl)methoxamine (3.00 g, 13.60 mmol) in DCM (60 mL) and H₂O (20 mL) was added Boc₂O (3.27 g, 15.00 mmol) at 0° C. under nitrogen atmosphere and the reaction mixture was stirred for 16 h at rt under nitrogen atmosphere. On completion, the reaction mixture was added water (100 mL) and extracted with CH₂Cl₂ (4×10 mL). The combined organic layers were washed with brine (3×10 mL), and dried over anhydrous Na₂SO₄. After filtration, the filtrate was concentrated under reduced pressure to the title compound (4.2 g, 96% yield) as a colorless oil; ¹H NMR (300 MHz, Chloroform-d) δ 7.52 (d, J 2.0 Hz, 1H), 7.38 (dd, J 8.2, 2.0 Hz, 1H), 7.26 (d, J 8.0 Hz, 1H), 5.04-5.00 (m, 1H), 4.34 (d, J 6.4 Hz, 2H), 1.45 (s, 9H); LC/MS (ESI, m/z): [(M+H)]⁺=319.9, 321.9.

Step 2—Tert-butyl N-[[2-chloro-4-(prop-1-en-2-yl)phenyl]methyl]carbamate. To a stirred solution of tert-butyl N-[(4-bromo-2-chlorophenyl)methyl]carbamate (3.60 g, 11.23 mmol) and 4,4,5,5-tetramethyl-2-(prop-1-en-2-yl)-1,3,2-dioxaborolane (2.26 g, 13.47 mmol) in DMA (15.00 mL) and H₂O (5.00 mL, 277.57 mmol) were added K₂CO₃ (3.10 g, 22.46 mmol) and XPhos palladium(II) biphenyl-2-amine chloride (441.73 mg, 0.56 mmol) in turns at rt under nitrogen atmosphere. The reaction mixture was stirred for 4 h at 90° C. under nitrogen atmosphere. On completion, the mixture was cooled to rt and the mixture was added water (100 mL) and extracted with EtOAc (3×30 mL). The combined organic layers were washed with brine (3×15 mL), and dried over anhydrous Na₂SO₄. After filtration, the filtrate was concentrated under reduced pressure. The residue was purified by reverse flash chromatography (column, Welflash™ C18-1,20-40 um, 330 g; Eluent A: Water (plus 10 mmol/L FA); Eluent B: ACN; Gradient 40% to 70% B in 30 min; Flow rate: 80 mL/min; Detector: 220/254 nm; desired fractions were collected at 56% B) and concentrated under reduced pressure to afford the title compound (2 g, 63% yield) as a white solid. ¹H NMR (300 MHz, Chloroform-d) δ 7.44-7.05 (m, 1H), 7.42-7.22 (m, 2H), 5.39 (s, 1H), 5.17-5.06 (m, 1H), 5.03 (s, 1H), 4.45-4.31 (m, 2H), 2.14 (s, 3H), 1.47 (s, 9H); LC/MS (ESI, m/z): [(M−H)]⁻=280.1.

Step 3—Tert-butyl N-[(2-chloro-4-isopropylphenyl)methyl]carbamate. To a stirred solution of tert-butyl N-[[2-chloro-4-(prop-1-en-2-yl)phenyl]methyl]carbamate (2.00 g, 7.10 mmol) in MeOH (10.00 mL) were added PtO₂ (162.7 mg, 0.71 mmol) at rt under nitrogen atmosphere. The reaction system was degassed under vacuum and purged with H₂ several times, then it was hydrogenated under H₂ balloon (˜1 atm) at 25° C. for 3 h. After completion of the reaction, Pd/C was filtered off through celite and the filter cake was washed with MeOH (3×10 mL). The corresponding filtrate was concentrated under reduced pressure to provide the title compound (1.6 g, 79% yield) as a white solid. ¹H NMR (300 MHz, Chloroform-d) δ 7.32-7.25 (m, 1H), 7.25-7.19 (m, 1H), 7.13-7.08 (m, 1H), 4.97 (s, 1H), 4.37 (d, J=6.1 Hz, 2H), 2.88 (p, J=6.9 Hz, 1H), 1.46 (s, 9H), 1.25 (d, J=6.9 Hz, 6H); LC/MS (ESI, m/z): [(M−H)]⁻=282.1.

Step 4—1-(2-Chloro-4-isopropylphenyl)methanamine hydrochloride. To a stirred solution of tert-butyl N-[(2-chloro-4-isopropylphenyl)methyl]carbamate (2 g, 7.06 mmol) in DCM (15.00 mL) was added a solution of 4 M HCl (gas) in 1,4-dioxane (5.00 mL) dropwise at rt under nitrogen atmosphere and the mixture was stirred for 2 h. On completion, the resulting mixture was concentrated under reduced pressure to afford the title compound (1.5 g, 97% yield) as a white solid. ¹H NMR (300 MHz, DMSO-d₆) δ 8.64 (s, br, 3H), 7.59 (d, J 8.0 Hz, 1H), 7.46-7.37 (m, 1H), 7.31 (dd, J 8.0, 1.7 Hz, 1H), 4.22-3.89 (m, 2H), 3.03-2.82 (m, 1H), 1.21 (d, J 6.8 Hz, 6H); LC/MS (ESI, m/z): [(M+H+CH₃CN)]⁺=225.2.

Step 5—Tert-butyl N-[(1S)-3-carbamoyl-1-[[(2-chloro-4-isopropylphenyl)methyl]carbamoyl]propyl]carbamate. To a stirred solution of 1-(2-chloro-4-isopropylphenyl)methanamine hydrochloride (1.40 g, 6.360 mmol) and (2S)-2-[(tert-butoxycarbonyl)amino]-4-carbamoylbutanoic acid (1.72 g, 7.00 mmol, CAS #13726-85-7) in DMA (15.00 mL) were added TEA (1.93 g, 19.08 mmol) and HATU (2.90 g, 7.63 mmol) in turns at 0° C. under nitrogen atmosphere and the mixture was stirred for 1 h at rt. On completion, the resulting mixture was concentrated under reduced pressure. The residue was purified by reverse flash chromatography (column, Welflash™ C18-1,20-40 um, 330 g; EluentA: Water (plus 10 mmol/1 FA); Eluent B: ACN; Gradient 30% to 60% B in 30 min; Flow rate: 80 mL/min; Detector: 220/254 nm; desired fractions were collected at 46% B) and concentrated under reduced pressure to afford the title compound (1.48 g, 57% yield) as a white solid. ¹H NMR (300 MHz, Chloroform-d) δ 7.31-7.18 (m, 2H), 7.12-7.06 (m, 2H), 6.18 (s, 1H), 5.71 (d, J 7.3 Hz, 1H), 5.62 (s, 1H), 4.58-4.40 (m, 2H), 4.19 (s, 1H), 2.96-2.67 (m, 1H), 2.42-2.25 (m, 2H), 2.19-2.04 (m, 1H), 2.08-1.84 (m, 1H), 1.42 (s, 9H), 1.23 (d, J 6.9 Hz, 6H); LC/MS (ESI, m/z): [(M−H)]⁻=410.1.

Step 6—(2S)-2-Amino-N-[(2-chloro-4-isopropylphenyl)methyl]pentanediamide hydrochloride. To a stirred solution of tert-butyl N-[(1S)-3-carbamoyl-1-[[(2-chloro-4-isopropylphenyl)methyl]carbamoyl]propyl]carbamate (1.48 g, 3.59 mmol) in DCM (20.00 mL) were added a solution of 4 M HCl (gas) in 1,4-dioxane (10.00 mL) dropwise at rt under nitrogen atmosphere and the mixture was stirred for 2 h. On completion, the resulting mixture was concentrated under reduced pressure to afford the title compound (1.2 g, 96% yield) as a white solid. ¹H NMR (300 MHz, DMSO-d₆) (9.15-9.05 (m, 1H), 8.41 (s, 3H), 7.51 (s, 1H), 7.41-7.30 (m, 2H), 7.22 (dd, J 7.9, 1.8 Hz, 1H), 6.94 (s, 1H), 4.37 (d, J 5.5 Hz, 2H), 3.91-3.86 (m, 1H), 2.99-2.83 (m, 1H), 2.29-2.18 (m, 2H), 2.06-1.93 (m, 2H), 1.20 (d, J 6.9 Hz, 6H); LC/MS (ESI, m/z): [(M+H)]⁺=312.3.

### (5S,8S,10aR)-5-[(tert-butoxycarbonyl)amino]-6-oxo-octahydro-1H-pyrrolo[1,2-a][1,5]diazocine-8-carboxylic acid (Intermediate AF)

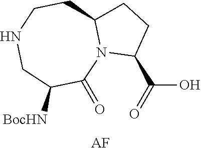

(5S,8S,10aR)-5-[(tert-butoxycarbonyl)amino]-6-oxo-octahydro-1H-pyrrolo[1,2-a][1,5]diazocine-8-carboxylic acid was synthesized as described in WO2011050068.

### (1s,4s)-4-[2-[1-(2,6-dioxopiperidin-3-yl)-3-methyl-2-oxo-1,3-benzodiazol-5-yl]ethyl]cyclohexane-1-carboxylic acid (Intermediate AG)

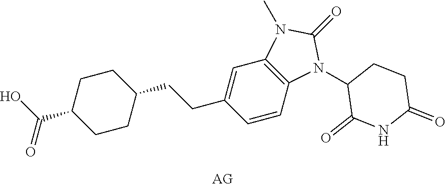 [SMILES: Cn1c(=O)n(C2CCC(=O)NC2=O)c2ccc(CC[C@H]3CC[C@@H](C(=O)O)CC3)cc21]

(400 MHz, Chloroform-d) δ 8.28 (s, 1H), 6.88 (dd, J=8.0, 1.6 Hz, 1H), 6.83 (d, J=1.5 Hz, 1H), 6.70 (d, J=8.0 Hz, 1H), 5.21 (dd, J=12.6, 5.4 Hz, 1H), 3.43 (s, 3H), 2.84-2.59 (m, 4H), 2.26-2.17 (m, 1H), 2.09-1.97 (m, 2H), 1.90-1.82 (m, 4H), 1.70-1.51 (m, 5H), 1.29-1.25 (m, 2H); LC/MS (ESI, m/z): [(M+H)]⁺=414.2.

### 5-[[tert-butoxy(hydroxy)phosphoryl]carbonyl]-1H-indole-2-carboxylic acid (Intermediate AH)

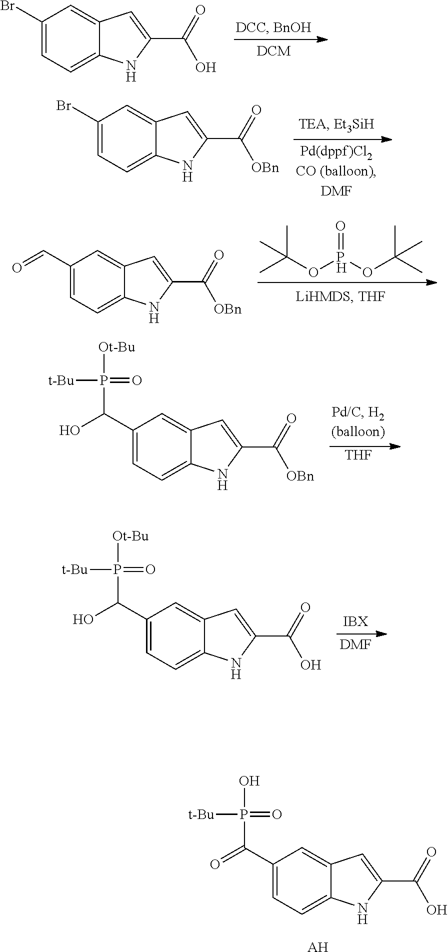

Step 1—Benzyl 5-bromo-1H-indole-2-carboxylate. To a stirred solution of 5-bromo-1H-indole-2-carboxylic acid (90.00 g, 374.91 mmol, CAS #7254-19-5) and benzyl alcohol (44.60 g, 412.40 mmol) in DCM (1800.00 mL) was added DCC (92.83 g, 449.90 mmol) in portions at rt under N₂ atmosphere and the mixture was stirred for 3 h. On completion, the resulting mixture was concentrated under reduced pressure. The residue was purified by silica gel column chromatography, eluted with PE/EtOAc (5:1), to afford the title compound (108 g, 87.25%) as a light pink solid; ¹H NMR (400 MHz, Chloroform-d) δ 9.15 (s, 1H), 7.80 (d, J=1.8 Hz, 1H), 7.49-7.43 (m, 2H), 7.43-7.33 (m, 4H), 7.26 (d, J=8.9 Hz, 1H), 7.19 (dd, J=2.1, 1.0 Hz, 1H), 5.39 (s, 2H); LC/MS (ESI, m z): [(M−1)]⁻=328.1, 330.1.

Step 2—Benzyl 5-formyl-1H-indole-2-carboxylate. To a stirred mixture of benzyl 5-bromo-1H-indole-2-carboxylate (30.00 g, 90.86 mmol) and Et₃SiH (58.46 g, 502.76 mmol) in DMF (600.00 mL) were added TEA (18.39 g, 181.72 mmol) and Pd(dppf)Cl₂·CH₂C1₂ (7.42 g, 9.09 mmol) at rt under nitrogen atmosphere. The reaction system was degassed under vacuum and purged with CO several times, then it was stirred under CO balloon for 3 h at 90° C. On completion, the reaction mixture was diluted with water (2 L) and extracted with EtOAc (3×600 mL). The combined organic layers were washed with brine (3×500 mL), and dried over anhydrous Na₂SO₄. After filtration, the filtrate was concentrated under reduced pressure and the residue was purified by silica gel column chromatography, eluted with 0%-20% EtOAc in PE, to afford the title compound (18 g, 71% yield) as a yellow solid. ¹H NMR (300 MHz, Chloroform-d) δ 10.03 (s, 1H), 9.34 (s, 1H), 8.22 (d, J=1.5 Hz, 1H), 7.89 (dd, J=8.7, 1.5 Hz, 1H), 7.55-7.33 (m, 7H), 5.42 (s, 2H); LC/MS (ESI, m z): [(M−1)]⁻=278.1.

Step 3—Benzyl 5-[[bis(tert-butoxy)phosphoryl](hydroxy)methyl]-1H-indole-2-carboxylate. To a stirred solution of di-tert-butyl phosphonate (62.58 g, 322.23 mmol) in THF (600 mL) was added a solution of 1 M LiHMDS (107.84 g, 644.48 mmol) in THF dropwise at −78° C. under nitrogen atmosphere. The resulting mixture was stirred for 45 min at −78° C. under nitrogen atmosphere. To the above solution was added a solution of benzyl 5-formyl-1H-indole-2-carboxylate (60.00 g, 214.83 mmol) in THF (500.00 mL) dropwise over 30 min at −78° C. The resulting mixture was stirred for additional 2 h at −50° C. On completion, the reaction was quenched with saturated aqueous NH₄Cl at 0° C. The resulting mixture was concentrated under reduced pressure and the residue was dissolved in water (900 mL). The mixture was extracted with EtOAc (3×300 mL), the combined organic layers were washed with brine (2×200 mL), and dried over anhydrous Na₂SO₄. After filtration, the filtrate was concentrated under reduced pressure. The residue was purified by silica gel column chromatography, eluted with 20%˜50% EtOAc, to afford the title compound (60 g, 59% yield) as a yellow solid, and 4 g of starting materials was recovered. ¹H NMR (300 MHz, Chloroform-d) δ 9.34 (s, 1H), 7.76 (d, J=2.6 Hz, 1H), 7.50-7.40 (m, 4H), 7.40-7.33 (m, 3H), 7.24 (dd, J=2.2, 0.9 Hz, 1H), 5.38 (s, 2H), 4.93 (d, J=9.2 Hz, 1H), 1.44 (s, 9H), 1.38 (s, 9H); LC/MS (ESI, m z): [(M−1)]⁻=472.2.

Step 4—5-[[bis(tert-butoxy)phosphoryl](hydroxy)methyl]-1H-indole-2-carboxylic acid. To a solution of benzyl 5-[[bis(tert-butoxy)phosphoryl](hydroxy)methyl]-1H-indole-2-carboxylate (60.00 g, 126.71 mmol) in THF (1.1 L) was added Pd/C (13.48 g, 12.67 mmol, 10 wt %) under nitrogen atmosphere. The reaction system was degassed under vacuum and purged with H₂ several times, then it was hydrogenated under H₂ balloon at rt for 4 h. After completion of the reaction, Pd/C was filtered off through celite and the filter pad was washed with DMF (200 mL), then the corresponding filtrate was concentrated under reduced pressure. The residue was purified by trituration with Et₂O to afford the title compound (44 g, 86%) as a white solid. ¹H NMR (300 MHz, DMSO-d₆) δ 12.93 (s, 1H), 11.65 (s, 1H), 7.74-7.60 (m, 1H), 7.40-7.25 (m, 2H), 7.06 (d, J=2.0 Hz, 1H), 5.76 (d, J=14.8 Hz, 1H), 4.68 (d, J=11.9 Hz, 1H), 1.36 (s, 9H), 1.29 (s, 9H); LC/MS (ESI, m z): [(M−1)]⁻=382.2.

Step 5—5-[[tert-butoxy(hydroxy)phosphoryl]carbonyl]-1H-indole-2-carboxylic acid. To a stirred mixture of 5-[[bis(tert-butoxy)phosphoryl](hydroxy)methyl]-1H-indole-2-carboxylic acid (17.00 g, 44.34 mmol) in DMF (150.00 mL) was added IBX (12.42 g, 44.34 mmol) at rt under nitrogen atmosphere and the reaction mixture was stirred for 1 h. The resulting mixture was filtered and the filter cake was washed with DMF (2×2 mL). The filtrate was adjusted to pH=9˜10 with TEA (˜6 mL) and concentrated under vacuum to ˜100 mL. The crude solution was purified by reverse phase flash chromatography (Column: WelFlash™ C18-I, 20-40 μm, 330 g; EluentA: Water (plus 10 mmol/L TEA); Eluent B: ACN; Gradient: 30%-45% B in 15 min; Flow rate: 80 mL/min; Detector: 220/254 nm; desired fractions were collected at 41% B) and concentrated under reduced pressure to afford the title compound (14 g, 72% yield) as a white solid. ¹H NMR (400 MHz, DMSO-d6) δ 12.02 (s, 1H), 8.93-8.89 (m, 1H), 8.02-7.95 (m, 1H), 7.48-7.41 (m, 1H), 7.26-7.16 (m, 1H), 3.01 (q, J=7.3 Hz, 6H), 1.31 (s, 9H), 1.17 (t, J=7.3 Hz, 9H); LC/MS (ESI, m z): [(M−1)]⁻=324.1.

### (2S)-2-amino-N-[(2-fluoro-4-isopropylphenyl)methyl]pentanediamide hydrochloride (Intermediate AI)

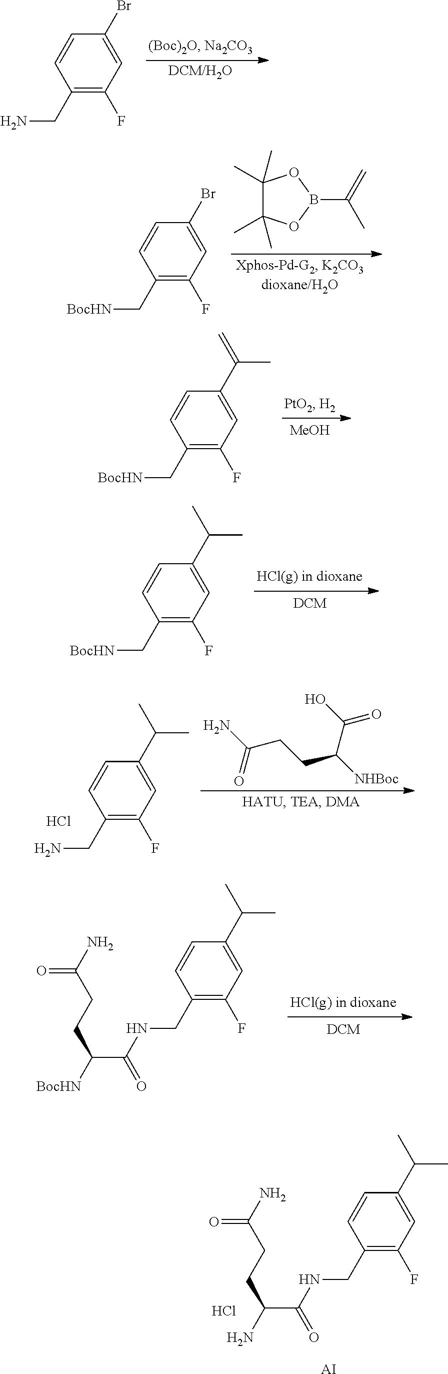

Step 1—Tert-butyl N-[(4-bromo-2-fluorophenyl)methyl]carbamate. To a stirred mixture of 1-(4-bromo-2-fluorophenyl)methanamine (10.00 g, 49.01 mmol) and Na₂CO₃ (15.58 g, 147.00 mmol) in DCM (100.00 mL) and H₂O (100.00 mL) was added Boc₂O (11.77 g, 53.93 mmol) at 0° C. under nitrogen atmosphere. The resulting mixture was stirred for 16 h at rt under nitrogen atmosphere. On completion, the resulting mixture was diluted with water (150 mL) and extracted with CH₂Cl₂ (2×100 mL). The combined organic layers were washed with brine (2×100 mL), and dried over anhydrous Na₂SO₄. After filtration, the filtrate was concentrated under reduced pressure to give the title compound (14 g, 94% yield) as a light yellow oil. ¹H NMR (300 MHz, Chloroform-d) δ 7.31-7.20 (m, 3H), 4.94 (s, 1H), 4.32 (d, J=6.2 Hz, 2H), 1.46 (s, 9H); LC/MS (ESI, m/z): [(M−H)]⁻=302.1, 304.1.

Step 2—Tert-butyl N-[[2-fluoro-4-(prop-1-en-2-yl)phenyl]methyl]carbamate. To a stirred mixture of tert-butyl N-[(4-bromo-2-fluorophenyl)methyl]carbamate (7.00 g, 23.01 mmol) and 4,4,5,5-tetramethyl-2-(prop-1-en-2-yl)-1,3,2-dioxaborolane (4.64 g, 27.61 mmol) in dioxane (100.00 mL) and H₂O (30.00 mL) were added K₂CO₃ (6.36 g, 46.02 mmol) and X-Phos aminobiphenyl palladium chloride precatalyst (905.38 mg, 1.15 mmol) in turns at rt under nitrogen atmosphere. The resulting mixture was then stirred for 4 h at 80° C. under nitrogen atmosphere. On completion, the resulting mixture was diluted with water (200 mL) and extracted with EtOAc (2×200 mL). The combined organic layers were washed with brine (2×200 mL), and dried over anhydrous Na₂SO₄. After filtration, the filtrate was concentrated under reduced pressure and the residue was purified by silica gel column chromatography, eluted with PE/EtOAc (100:1 to 2:1), to afford the title compound (5.8 g, 95% yield) as a brown solid. ¹H NMR (300 MHz, Chloroform-d) δ 7.32-7.23 (m, 1H), 7.20 (dd, J=7.9, 1.7 Hz, 1H), 7.12 (dd, J=11.7, 1.7 Hz, 1H), 5.38-5.35 (m, 1H), 5.11 (s, 1H), 4.91 (s, 1H), 4.34 (d, J=6.1 Hz, 2H), 2.11 (s, 3H), 1.45 (s, 9H). LC/MS (ESI, m/z): [(M+H)]⁺=266.2.

Step 3—Tert-butyl N-[(2-fluoro-4-isopropylphenyl)methyl]carbamate. To a solution of tert-butyl N-[[2-fluoro-4-(prop-1-en-2-yl)phenyl]methyl]carbamate (5.80 g, 21.86 mmol) in THF (60.00 mL, 832.14 mmol) was added PtO₂ (595.68 mg, 2.62 mmol) under nitrogen atmosphere. The mixture was hydrogenated at rt for 4 h under hydrogen atmosphere using a hydrogen balloon. On completion, the mixture was filtered through a celite pad and the filtrate was concentrated under reduced pressure. The title compound (5.3 g, 91% yield) as a light yellow solid. ¹H NMR (400 MHz, Chloroform-d) δ 7.27-7.18 (m, 1H), 6.96 (dd, J=7.8, 1.8 Hz, 1H), 6.90 (dd, J=11.5, 1.7 Hz, 1H), 4.86 (s, 1H), 4.32 (d, J=6.0 Hz, 2H), 2.88 (hept, J=6.9 Hz, 1H), 1.45 (s, 9H), 1.23 (d, J=6.9 Hz, 6H); LC/MS (ESI, m/z): [(M+H)]⁺=268.2.

Step 4—1-(2-fluoro-4-isopropylphenyl)methanamine hydrochloride. To a stirred solution of tert-butyl N-[(2-fluoro-4-isopropylphenyl)methyl]carbamate (5.30 g, 19.83 mmol) in dioxane (50.00 mL) was added HCl (gas) in 1,4-dioxane (50.00 mL) dropwise at 0° C. under nitrogen atmosphere. The resulting mixture was stirred for 2 h at rt under nitrogen atmosphere. On completion, the reaction mixture was concentrated under reduced pressure to afford the title compound (4.8 g, 95% yield) as a light yellow solid. ¹H NMR (400 MHz, Chloroform-d) δ 8.59 (s, 3H), 7.51-7.47 (m, 1H), 7.04-6.98 (m, 1H), 6.95 (d, J=10.6 Hz, 1H), 4.11 (s, 2H), 2.87 (p, J=6.8 Hz, 1H), 1.20 (d, J=6.7 Hz, 6H); LC/MS (ESI, m/z): [(M+H)]+=168.2.

Step 5—tert-butyl N-[(1S)-3-carbamoyl-1-[[(2-fluoro-4-isopropylphenyl)methyl]carbamoyl]propyl]carbamate. To a stirred solution of 1-(2-fluoro-4-isopropylphenyl)methanamine hydrochloride (2.00 g, 9.82 mmol) and (2S)-2-[(tert-butoxycarbonyl)amino]-4-carbamoylbutanoic acid (2.90 g, 11.78 mmol, CAS #13726-85-7) in DMF (20.00 mL) were added TEA (2.98 g, 29.45 mmol) and HATU (4.85 g, 12.76 mmol) at rt under nitrogen atmosphere and the mixture was stirred for 2 h. The residue was purified by reverse flash chromatography (column, Column: WelFlash™ C18-I; mobile phase, MeCN in water (10 mmol/LNH₄HCO₃), 15% to 65% gradient in 25 min; detector, UV 220 nm) to afford the title compound (3.1 g, 80% yield) as a light yellow solid. ¹H NMR (300 MHz, Chloroform-d) δ 7.29 (s, 1H), 7.22 (t, J=7.9 Hz, 1H), 6.95 (dd, J=7.8, 1.7 Hz, 1H), 6.90 (dd, J=11.4, 1.7 Hz, 1H), 6.36 (s, 1H), 5.81 (d, J=11.6 Hz, 2H), 4.44 (t, J=5.5 Hz, 2H), 4.21 (s, 1H), 2.88 (hept, J=6.9 Hz, 1H), 2.48-2.18 (m, 1H), 2.14-2.01 (m, 2H), 2.00-1.86 (m, 1H), 1.42 (s, 9H), 1.23 (d, J=6.9 Hz, 6H); LC/MS (ESI, m/z): [(M+1)]+=396.4.

Step 6—(2S)-2-amino-N-[(2-fluoro-4-isopropylphenyl)methyl]pentanediamide hydrochloride. To a stirred mixture of tert-butyl N-[(1S)-3-carbamoyl-1-[[(2-fluoro-4-isopropylphenyl)methyl]carbamoyl]propyl]carbamate (3.10 g, 7.84 mmol) in dioxane (30.00 mL) was added dropwise HCl (gas) in 1,4-dioxane (30.00 mL) at 0° C. under nitrogen atmosphere. The resulting mixture was stirred for 2 h at rt under nitrogen atmosphere. The resulting mixture was concentrated under reduced pressure to afford the title compound (2.5 g, 96% yield) as a brown solid. ¹H NMR (300 MHz, Methanol-d₄) δ 7.30 (t, J=7.9 Hz, 1H), 7.03 (dd, J=7.8, 1.7 Hz, 1H), 6.97 (dd, J=11.5, 1.7 Hz, 1H), 4.44 (s, 2H), 3.92 (t, J=6.2 Hz, 1H), 3.31 (p, J=1.7 Hz, 2H), 2.89 (h, J=6.9 Hz, 1H), 2.44-2.39 (m, 2H), 2.20-2.03 (m, 2H), 1.22 (d, J=6.9 Hz, 6H); LC/MS (ESI, m/z): [(M+1)]=296.3.

### (2S)-2-Amino-N-[(3-chloro-4-isopropylphenyl)methyl]pentanediamide hydrochloride (Intermediate AJ)

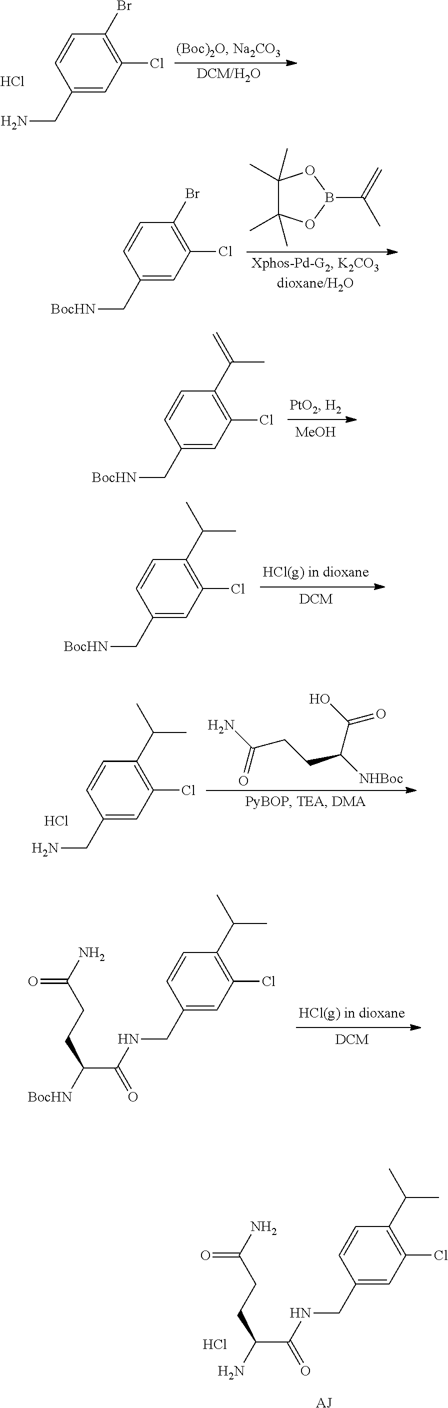

Step 1—Tert-butyl N-[(4-bromo-3-chlorophenyl)methyl]carbamate. To a stirred solution of 1-(4-bromo-3-chlorophenyl)methanamine (5.00 g, 22.68 mmol) and Na₂CO₃ (7.21 g, 68.03 mmol) in DCM (80 mL) and H₂O (50 mL) was added Boc₂O (5.44 g, 24.94 mmol) at 0° C. under nitrogen atmosphere. The resulting mixture was stirred for 2 h at rt under nitrogen atmosphere. On completion, the reaction mixture was diluted with water (150 mL) and extracted with CH₂Cl₂ (2×100 mL). The combined organic layers were washed with brine (2×100 mL), and dried over anhydrous Na₂SO₄. The resulting mixture was concentrated under reduced pressure to afford the title compound (4.18 g, 58% yield) as a light yellow oil. ¹H NMR (300 MHz, Chloroform-d) δ 7.56 (d, J=8.2 Hz, 1H), 7.38 (s, 1H), 7.05 (d, J=8.2 Hz, 1H), 4.99-4. 88 (m, 1H), 4.25 (d, J=5.9 Hz, 2H), 1.47 (s, 9H); LC/MS (ESI, m/z): [(M+H)]+=319.9, 320.9.

Step 2—Tert-butyl N-[[3-chloro-4-(prop-1-en-2-yl)phenyl]methyl]carbamate. To a stirred mixture of tert-butyl N-[(4-bromo-3-chlorophenyl)methyl]carbamate (3.78 g, 11.79 mmol) and 4,4,5,5-tetra methyl-2-(prop-1-en-2-yl)-1,3,2-dioxaborolane (2.38 g, 14.15 mmol) in DMA (40.00 mL) and H₂O (20.00 mL) were added K₂CO₃ (3.26 g, 23.58 mmol) and XPhos palladium(II) biphenyl-2-amine chloride (0.46 g 0.59 mmol) in turns at rt under nitrogen atmosphere. The resulting mixture was stirred for 2 h at 80° C. un der nitrogen atmosphere. On completion, the mixture was cooled to rt and concentrated under reduced pressure. The residue was purified by reverse phase flash chromatography (Column: WelFlash™ C18-I, 20-40 m, 330 g; Eluent A: Water (plus 10 mmol/L NH₄HCO₃); Eluent B: ACN; Gradient: 30%-60% B in 30 min; Flow rate: 60 mL/min; Detector: 220/254 nm; desired fractions were collected at 45% B) and concentrated under reduced pressure to afford the title compound (2.35 g, 71% yield) as a yellow solid. ¹H NMR (300 MHz, Chloroform-d) δ 7.29 (s, 1H), 7.20-7.09 (m, 2H), 5.29-5.21 (m, 1H), 5.04-4.92 (m, 1H), 4.92-4 0.86 (m, 1H), 4.29 (d, J=5.9 Hz, 2H), 2.10 (s, 3H), 1.48 (s, 9H); LC/MS (ESI, m/z): [(M−H)]⁻=280.1.

Step 3—Tert-butyl N-[(3-chloro-4-isopropylphenyl)methyl]carbamate. To a stirred solution of tert-butyl N-[[3-chloro-4-(prop-1-en-2-yl)phenyl]methyl]carbamate (2.35 g, 8.34 mmol) in MeOH (30.00 mL, 741.00 mmol) was added PtO₂ (300.00 mg, 1321.10 mmol) under argon atmosphere. The reaction system was degassed under vacuum and purged with H₂ several times, then it was hydrogenated under H₂ balloon (1 atm) at rt for 1 h. After completion of the reaction, Pd/C was filtered off through celite and the filter cake was washed with MeOH (3×30.00 mL). The filtrate was concentrated under reduced pressure to provide the title compound (2.3 g, 97.18%) as a yellow oil. ¹H NMR (300 MHz, Chloroform-d) δ 7.29-7. 23 (m, 2H), 7.18-7.13 (m, 1H), 4.90-4.79 (m, 1H), 4.26 (d, J=5.9 Hz, 2H), 3.44-3.39 (m, 1H), 1.47 (s, 9H), 1.24 (d, J=6.9 Hz, 6H); LC/MS (ESI, m/z): [(M−H)]⁻=282.1.

Step 4—1-(3-Chloro-4-isopropylphenyl)methanamine hydrochloride. To a stirred solution of tert-butyl N-[(3-chloro-4-isopropylphenyl)methyl]carbamate (2.31 g, 8.14 mmol) in DCM (30 mL) was added a solution of 4 M HCl (gas) in 1,4-dioxane (10 mL) at 0° C. under nitrogen atmosphere. The resulting mixture was stirred for 2 h at rt under nitrogen atmosphere. On completion, the mixture was concentrated under reduced pressure to afford the title compound (1.76 g, 98% yield) as a yellow oil. ¹H NMR (300 MHz, DMSO-d₆) δ 8.42-8.33 (m, 3H), 7.59-7.55 (m, 1H), 7.46-7.42 (m, 2H), 4.02-3.96 (m, 2H), 3.58-3.55 (m, 1H), 1.20 (d, J=6.9 Hz, 6H); LC/MS (ESI, m/z): [(M+H)]⁺=184.1.

Step 5—Tert-butyl N-[(1S)-3-carbamoyl-1-[[(3-chloro-4-isopropylphenyl)methyl]carbamoyl]propyl]carbamate. To a stirred solution of 1-(3-chloro-4-isopropylphenyl)methanamine hydrochloride (1.8 6 g, 8.45 mmol) and (2S)-2-[(tert-butoxycarbonyl)amino]-4-carbamoylbutanoic acid (2.50 g, 10.14 mmol, CAS #13726-85-7) in DMA (20.00 mL) were added TEA (2.56 g, 25.35 mmol) and HATU (4.18 g, 10.99 mmol) in turns at 0° C. under nitrogen atmosphere. The resulting mixture was stirred for 1 h at rt under nitrogen atmosphere. On completion, the reaction mixture was concentrated under reduced pressure. The residue was purified by reverse phase flash chromatography (Column: WelFlash™ C18-I, 20-40 m, 330 g; EluentA: Water (plus 10 mmol/L NH₄HCO₃); Eluent B: ACN; Gradient: 35%-60% B in 30 min; Flow rate: 80 mL/min; Detector: 220/254 nm; desired fractions were collected at 48% B) and concentrated under reduced pressure to afford the title compound (2.64 g, 76% yield) as a white solid. ¹H NMR (400 MHz, Chloroform-d) δ 7.28-7.23 (m, 2H), 7.17-7.13 (m, 1H), 7.08 (s, 1H), 6.08 (s, 1H), 5.72 (s, 1H), 5.53 (s, 1H), 4.48-4.31 (m, 2H), 4.25-4.18 (m, 1H), 3.40-3.35 (m, 1H), 2.45-2.39 (m, 2H), 2.19-1.94 (m, 2H), 1.44 (s, 9 H), 1.24 (d, J=6.9 Hz, 6H); LC/MS (ESI, m/z): [(M+H)]⁺=412.2.

Step 6—(2S)-2-Amino-N-[(3-chloro-4-isopropylphenyl)methyl]pentanediamide hydrochloride. To a stirred solution of tert-butyl N-[(1S)-3-carbamoyl-1-[[(3-chloro-4-isopropylphenyl)methyl]carbamoyl]propyl]carbamate (2.58 g, 6.26 mmol) in DCM (30 mL) was added a solution of 4 M HCl (gas) in 1,4-dioxane (10 mL) dropwise at 0° C. under nitrogen atmosphere. The resulting mixture was stirred for 2 h at rt under nitrogen atmosphere. On completion, the reaction mixture was concentrated under reduced pressure to afford the title compound (2.10 g, 96% yield) as a white solid. ¹H NMR (300 MHz, DMSO-d₆) δ 9.07 (s, 1H), 8.35-8.30 (m, 3H), 7.48 (s, 1H), 7.43-7.31 (m, 2H), 6.93 (s, 1H), 4.44-4.17 (m, 2H), 3.90-3.78 (m, 1H), 3.31-3.25 (m, 1H), 2.25-2.17 (m, 2H), 2.01-1.92 (m, 2H), 1.18 (d, J=6.8 Hz, 6H); LC/MS (ESI, m/z): [(M+H)]+=312.2.

### (4S)-4-Amino-5-(2-chloro-3-[5-[1-(2,6-dioxopiperidin-3-yl)-3-methyl-2-oxo-1,3-benzodiazol-5-yl]pentyl]phenoxy)pentanamide hydrochloride (Intermediate AK)

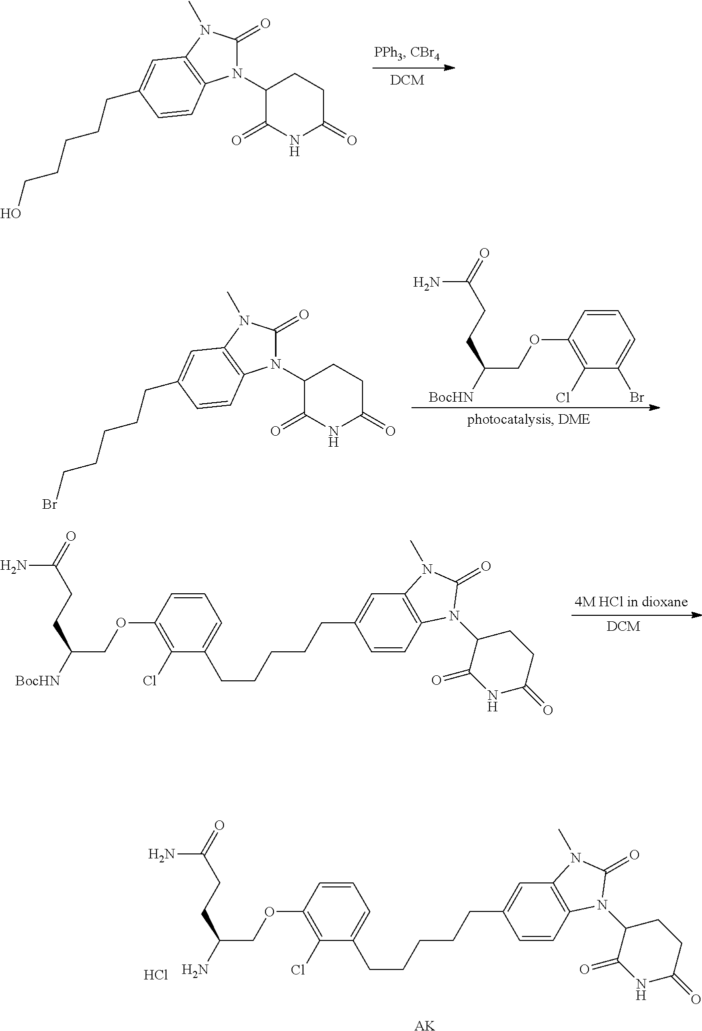

Step 1—3-[5-(5-Bromopentyl)-3-methyl-2-oxo-1,3-benzodiazol-1-yl]piperidine-2,6-dione. To a stirred solution of 3-[5-(5-hydroxypentyl)-3-methyl-2-oxo-1,3-benzodiazol-1-yl]piperidine-2,6-dione (2.00 g, 5.79 mmol) and CBr₄ (5.76 g, 17.37 mmol) in DCM (40.00 mL) was added PPh₃ (3.80 g, 14.48 m mol) at 0° C. under nitrogen atmosphere. The resulting mixture was stirred for 3 h at rt under nitrogen atmosphere. On completion, the reaction mixture was concentrated under reduced pressure. The residue was purified by reverse flash chromatography (Column: WelFlash™ C18-I, 20-40 μm, 330 g; EluentA: Water (plus 10 mmol/L FA); Eluent B: ACN; Gradient: 30%-60% B in 30 min; Flow rate: 80 mL/min; Detect or: 220/254 nm; desired fractions were collected at 49% B) and concentrated under reduced pressure to afford the title compound (1.78 g, 75% yield) as a light yellow oil. ¹H NMR (300 MHz, Chloroform-d) δ 8. 23 (s, 1H), 6.93-6.82 (m, 2H), 6.72 (d, J=8.0 Hz, 1H), 5.22 (dd, J=12.5, 5.4 Hz, 1H), 3.44 (s, 3H), 3.01-2.83 (m, 2H), 2.81-2.62 (m, 3H), 2.28-2.12 (m, 1H), 1.96-1.85 (m, 2H), 1.75-1.63 (m, 4H), 1.57-1.42 (m, 2H); LC/MS (ESI, m/z): [(M+H)]⁺=408.1, 410.2.

Step 2—Tert-butyl N-[(2S)-4-carbamoyl-1-(2-chloro-3-[5-[1-(2,6-dioxopiperidin-3-yl)-3-methyl-2-oxo-1,3-benzodiazol-5-yl]pentyl]phenoxy)butan-2-yl]carbamate. To an 8 mL vial equipped with a stir bar was added photocatalyst Ir[DF(CF₃)PPY]₂(DTBPY)PF₆ (212.95 mg, 0.19 mmol), 3-[5-(5-bromopent yl)-3-methyl-2-oxo-1,3-benzodiazol-1-yl]piperidine-2,6-dione (1.55 g, 3.80 mmol), tert-butyl N-[(2S)-1-(3-bromo-2-chlorophenoxy)-4-carbamoylbutan-2-yl]carbamate (2.40 g, 5.69 mmol, synthesized via Steps 1-3 of Intermediate C), tris(propan-2-yl)silane (943.98 mg, 0.005 mmol), and Na₂CO₃ (1.21 g, 11.39 mmol). The vial was sealed and placed under nitrogen before 4 mL of DME was added. To a separate vial was added 1,2-dimethoxyethane dihydrochloride nickel (41.71 mg, 0.19 mmol) and 4,4′-di-tert-butyl-2,2′-bipyridine (50.95 mg, 0.19 mmol). The catalyst vial was sealed, purged with nitrogen then to it was added 8 mL of DME. The precatalyst solution was sonicated or stirred for 5 min, after which, 2 mL (0.5 mol % catalyst, 2.5 μmol, 0.005 equiv.) was syringed into the reaction vessel. The solution was degassed by sparging with nitrogen while stirring for 10 minutes before sealing with parafilm. The reaction was stirred and irradiate d with a 34 W blue LED lamp, with cooling fan to keep the reaction temperature at 25° C., for 16 hours. The reaction was quenched by exposure to air and concentrated in vacuo. The residue was purified by reverse flash chromatography (Column: WelFlash™ C18-I, 20-40 m, 120 g; EluentA: Water (plus 10 mmol/L FA); Eluent B: ACN; Gradient: 30%-50% B in 30 min; Flow rate: 60 mL/min; Detector: 220/254 nm; desired fractions were collected at 42% B) and concentrated under reduced pressure to afford the title compound (579 mg, 23% yield) as an off-white solid. ¹H NMR (300 MHz, Chloroform-d) δ 8.44 (s, 1H), 7.12 (t, J=7.9 Hz, 1H), 7.00-6.79 (m, 4H), 6.79-6.59 (m, 2H), 6.35 (s, 1H), 5.50 (s, 1H), 5.27-5.15 (m, 2H), 4.0 9-4.02 (m, 2H), 3.43 (s, 3H), 3.02-2.81 (m, 2H), 2.80-2.63 (m, 2H), 2.42-2.33 (m, 2H), 2.27-2.16 (m, 1H), 2.09-2.04 (m, 1H), 1.73-1.62 (m, 8H), 1.46 (s, 9H), 1.45-1.39 (m, 2H); LC/MS (ESI, m/z): [(M+H)]+=670.3.

Step 3—(4S)-4-Amino-5-(2-chloro-3-[5-[1-(2,6-dioxopiperidin-3-yl)-3-methyl-2-oxo-1,3-benzodiazol-5-yl]pentyl]phenoxy)pentanamide hydrochloride. To a stirred solution of tert-butyl N-[(2S)-4-carbamoyl-1-(2-chloro-3-[5-[1-(2,6-dioxopiperidin-3-yl)-3-methyl-2-oxo-1,3-benzodiazol-5-yl]pentyl]phen oxy)butan-2-yl]carbamate (563.50 mg, 0.84 mmol) in DCM (5.00 mL) was added a solution of 4 M HCl (gas) in 1,4-dioxane (2.00 mL, 35.04 mmol) dropwise at 0° C. under nitrogen atmosphere. The resulting mixture was stirred for 2 h at rt under nitrogen atmosphere. On completion, the reaction mixture was concentrated under reduced pressure to afford the title compound (503.2 mg, 99% yield) as a white solid. ¹H NM R (300 MHz, DMSO-d₆) δ 11.06 (s, 1H), 8.54-8.30 (m, 3H), 7.46 (s, 1H), 7.24-7-18 (m, 1H), 7.06-6.88 (m, 5H), 6.88-6.81 (m, 1H), 5.34 (dd, J=12.8, 5.3 Hz, 1H), 4.43-4.08 (m, 2H), 3.31 (s, 3H), 2.99-2.80 (m, 1 H), 2.76-2.54 (m, 6H), 2.37-2.26 (m, 2H), 2.03-1.90 (m, 3H), 1.69-1.49 (m, 4H), 1.43-1.19 (m, 2H); LC/M S (ESI, m/z): [(M+H)]⁺=570.3.

### Tert-butyl N-[(5S,8S,10aR)-8-[[(1S)-3-carbamoyl-1-(pyridin-2-yl)propyl]carbamoyl]-6-oxo-octahydro-1H-pyrrolo[1,2-a][1,5]diazocin-5-yl]carbamate (Intermediate AL)

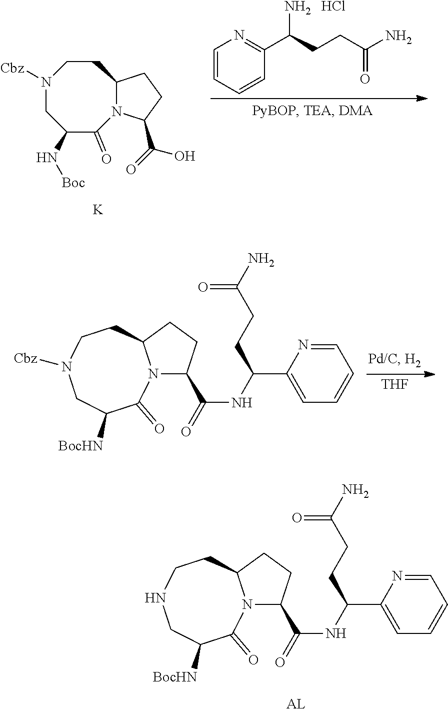

Step 1—Benzyl (5S,8S,10aR)-5-[(tert-butoxycarbonyl)amino]-8-[[(1S)-3-carbamoyl-1-(pyridin-2-yl)propyl]carbamoyl]-6-oxo-octahydropyrrolo[1,2-a][1,5]diazocine-3-carboxylate. To a solution of (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (400.00 mg, 0.87 mmol, Intermediate K), (4S)-4-amino-4-(pyridin-2-yl)butanamide (155.33 mg, 0.87 mmol) in DMA (5.00 mL) was added TEA (350.81 mg, 3.47 mmol) at rt. Then PyBOP (676.54 mg, 1.30 mmol) was added at rt and the mixture was stirred for 1 h at rt under nitrogen atmosphere. On completion, the resulting mixture was purified by reverse phase flash chromatography (Column: WelFlash™ C18-I, 20-40 ?m, 330 g; Eluent A: Water (plus 10 mmol/L FA); Eluent B: ACN; Gradient: 25%-45% B in 20 min; Flow rate: 80 mL/min; Detector: 220/254 nm; desired fractions were collected at 33% B) and concentrated under reduced pressure to afford the title compound (330 mg, 61% yield) as a white solid. ¹H NMR (300 MHz, Chloroform-d) δ 8.52 (d, J=4.8 Hz, 1H), 7.75-7.61 (m, 1H), 7.59-7. 17 (m, 10H), 7.12 (s, 1H), 5.67 (d, J=7.2 Hz, 2H), 5.25-5.19 (m, 2H), 5.13-5.02 (m, 1H), 4.84-4.74 (m, 1 H), 4.50-4.33 (m, 1H), 4.27-4.16 (m, 1H), 3.84-3.67 (m, 2H), 3.61-3.49 (m, 1H), 3.21-3.09 (m, 1H), 2.60-2.46 (m, 1H), 2.32-2.22 (m, 2H), 2.17-2.05 (m, 3H), 1.87-1.72 m, 2H), 1.45 (s, 9H); LC/MS (ESI, m/z): [(M+H)]+=623.4.

Step 2—Tert-butyl N-[(5S,8S,10aR)-8-[[(1S)-3-carbamoyl-1-(pyridin-2-yl)propyl]carbamoyl]-6-oxo-octahydro-1H-pyrrolo[1,2-a][1,5]diazocin-5-yl]carbamate. To a stirred solution of benzyl (5S,8S,10aR)-5-[(tert-butoxycarbonyl)amino]-8-[[(1S)- 3-carbamoyl-1-(pyridin-2-yl)propyl]carbamoyl]-6-oxo-octahydropyrrolo[1,2-a][1,5]diazocine-3-carboxylate (330.00 mg, 0.53 mmol) in THF (10.00 mL) was added Pd/C (100.00 mg, 0.94 mmol) at rt under nitrogen atmosphere. The resulting mixture was purged with hydrogen 3 times and stirred for overnight at rt under hydrogen atmosphere. On completion, the resulting mixture was filtered and the filter cake was washed with THF (3×5 mL). The filtrate was concentrated under reduced pressure and the residue was purified by reverse phase flash chromatography (Column: WelFlash™ C18-I, 20-40 ?m, 120 g; Eluent A: Water (plus 10 mmol/L FA); Eluent B: ACN; Gradient: 10%-30% B in 20 min; Flow rate: 55 mL/min; Detector: 220/254 nm; desired fractions were collected at 20% B) a nd concentrated under reduced pressure to afford the title compound (180 mg, 70% yield) as a white solid. ¹H NMR (400 MHz, DMSO-d₆) δ 9.11-8.99 (m, 1H), 8.53-8.47 (m, 1H), 7.77-7.69 (m, 1H), 7.32 (d, J=7 0.8 Hz, 1H), 7.29-7.20 (m, 2H), 6.99 (d, J=7.7 Hz, 1H), 6.75 (s, 1H), 4.83-4.73 (m, 1H), 4.72-4.59 (m, 1H), 4.45-4.32 (m, 2H), 3.21-3.13 (m, 1H), 2.91-2.82 (m, 1H), 2.81-2.70 (m, 2H), 2.36-2.28 (m, 1H), 2.19-1. 97 (m, 5H), 1.88-1.77 (m, 3H), 1.75-1.66 (m, 1H), 1.53-1.45 (m, 1H), 1.39 (s, 9H); LC/MS (ESI, m/z): [(M+H)]⁺=489.3.

### (5S,8S,10aR)-5-[(tert-butoxycarbonyl)amino]-6-oxo-3-[2-[(1s,4s)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocine-8-carboxylic acid (Intermediate AM)

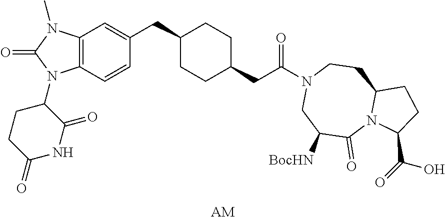

### (4S)-4-amino-5-(3-methanesulfonylphenoxy)pentanamide hydrochloride (Intermediate AN)

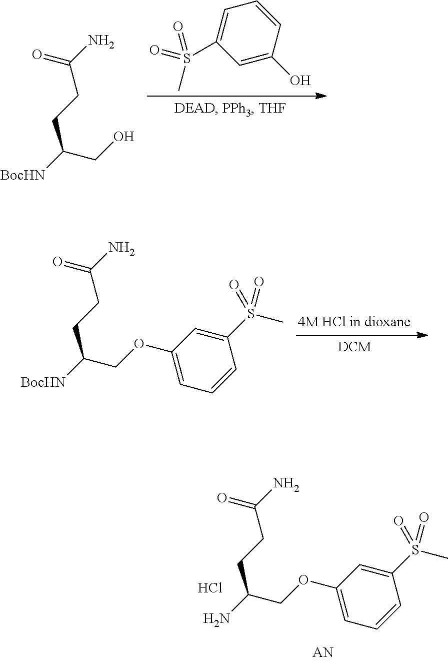 [SMILES: CC(C)(C)OC(=O)N[C@H](CO)CCC(N)=O ; CS(=O)(=O)c1cccc(O)c1 ; CC(C)(C)OC(=O)N[C@@H](CCC(N)=O)COc1cccc(S(C)(=O)=O)c1 ; Cl ; CS(=O)(=O)c1cccc(OC[C@@H](N)CCC(N)=O)c1]

Step 1—Tert-butyl N-[(2S)-4-carbamoyl-1-(3-methanesulfonylphenoxy)butan-2-yl]carbamate. To a stirred mixture of tert-butyl N-[(2S)-4-carbamoyl-1-hydroxybutan-2-yl]carbamate (5.40 g, 23.25 mmol, CAS #133565-42-1) and 3-methanesulfonylphenol (2.00 g, 11.61 mmol) in THF (50.00 mL) were added PPh₃ (4.57 g, 17.42 mmol) and DEAD (3.03 g, 17.42 mmol) in turns at 0° C. under nitrogen atmosphere. The resulting mixture was stirred for 3 h at rt under nitrogen atmosphere. On completion, the reaction mixture was concentrated under reduced pressure. The residue was purified by silica gel column chromatography, eluted with EA, to afford the title compound (1 g, 22% yield) as a white solid. ¹H NMR (400 MHz, DMSO-d₆) δ 7.61-7.53 (m, 1H), 7.52-7.46 (m, 1H), 7.45-7.40 (m, 1H), 7.33-7.29 (m, 1H), 7.29-7.25 (m, 1H), 6.89 (d, J=8.5 Hz, 1H), 6.74 (s, 1H), 4.01-3.94 (m, 2H), 3.80-3.70 (m, 1H), 3.23 (s, 3H), 2.18-2.08 (m, 2H), 1.89-1.77 (m, 1H), 1.70-1.58 (m, 1H), 1.39 (s, 9H); LC/MS (ESI, m/z): [(M+H)]+=387.2.

Step 2—(4S)-4-amino-5-(3-methanesulfonylphenoxy)pentanamide hydrochloride. To a stirred solution of tert-butyl N-[(2S)-4-carbamoyl-1-(3-methanesulfonylphenoxy)butan-2-yl]carbamate (300.00 mg, 0.78 mmol) in DCM (4.00 mL) was added HCl (gas) in 1,4-dioxane (2.00 mL, 35.04 mmol) at rt under nitrogen atmosphere and the reaction mixture was stirred for 1 h. On completion, the reaction mixture w as concentrated under reduced pressure to the title compound (280 mg, crude) as a white solid. ¹H NMR (400 MHz, Methanol-d₄) δ 7.67-7.57 (m, 3H), 7.44-7.36 (m, 1H), 4.42-4.33 (m, 1H), 4.27-4.18 (m, 1H), 3. 77-3.70 (m, 1H), 3.16 (s, 3H), 2.62-2.44 (m, 2H), 2.14-2.04 (m, 2H); LC/MS (ESI, m/z): [(M+H)]⁺=28 7.0.

### tert-butyl N-[(2S,11S)-2-[[(1S)-3-carbamoyl-1-[[(4-methanesulfonylphenyl)methyl]carbamoyl]propyl]carbamoyl]-12-oxo-6-(5-oxopentyl)-1-azatricyclo[6.4.1.0\hat{ }[4,13]]trideca-4(13),5,7-trien-11-yl]carbamate (Intermediate AO)

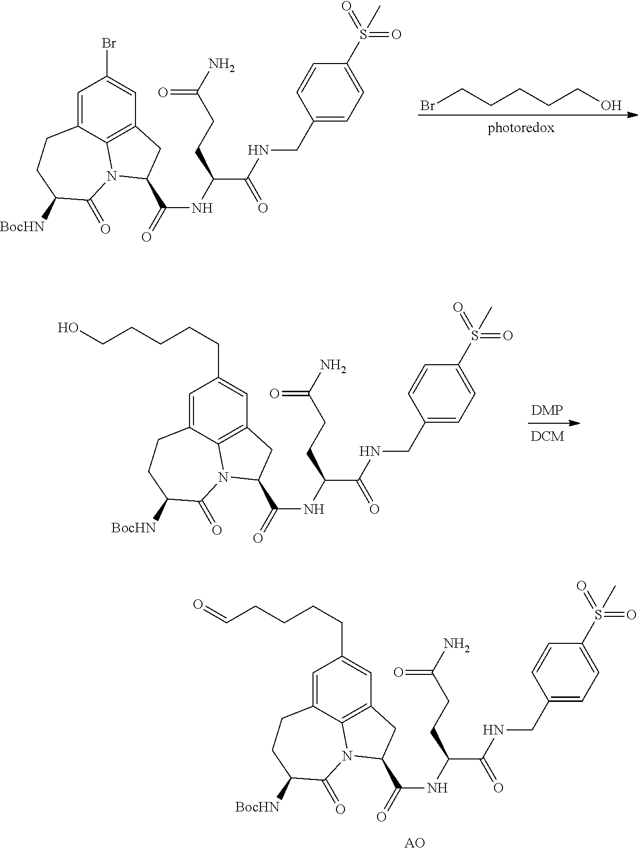 [SMILES: CC(C)(C)OC(=O)N[C@H]1CCc2cc(Br)cc3c2N(C1=O)[C@H](C(=O)N[C@@H](CCC(N)=O)C(=O)NCc1ccc(S(C)(=O)=O)cc1)C3 ; OCCCCCBr ; CC(C)(C)OC(=O)N[C@H]1CCc2cc(CCCCCO)cc3c2N(C1=O)[C@H](C(=O)N[C@@H](CCC(N)=O)C(=O)NCc1ccc(S(C)(=O)=O)cc1)C3 ; CC(C)(C)OC(=O)N[C@H]1CCc2cc(CCCCC=O)cc3c2N(C1=O)[C@H](C(=O)N[C@@H](CCC(N)=O)C(=O)NCc1ccc(S(C)(=O)=O)cc1)C3]

Step 1—tert-butyl N-[(2S,11S)-2-[[(1S)-3-carbamoyl-1-[[(4-methanesulfonylphenyl)methyl]carbamoyl]propyl]carbamoyl]-6-(5-hydroxypentyl)-12-oxo-1-azatricyclo[6.4.1.0\hat{ }[4,13]]trideca-4(13),5,7-trien-11-yl]carbamate. To a mixture of dtbbpy (1.86 mg, 0.007 mmol) in DME (1.00 mL) was added 1,2-dimethoxyethane dihydrochloride nickel (1.52 mg, 0.007 mmol) at rt under nitrogen atmosphere. Then to the mixture was bubbled with nitrogen for 10 min. To a mixture of tert-butyl N-[(2S,11S)-6-bromo-2-[[(1S)-3-carbamoyl-1-[[(4-methanesulfonylphenyl)methyl]carbamoyl]propyl]carbamoyl]-12-oxo-1-azatricyclo[6.4.1.0\hat{ }[4,13]]trideca-4(13),5,7-trien-11-yl]carbamate (500.00 mg, 0.69 mmol, Intermediate AV) and 5-bromopentan-1-ol (139.08 mg, 0.83 mmol) in DME (15.00 mL) were added tris(trimethylsilyl)silane (172.53 mg, 0.69 mmol), Na₂CO₃ (220.61 mg, 2.08 mmol), IR[DF(CF₃)PPY]₂(DTBPY)PF₆ (7.78 mg, 0.007 mmol) and the catalyst mixture dropwise at rt under nitrogen atmosphere. The final reaction mixture was irradiated with ultraviolet lamp for 16 h at rt. On completion, the resulting mixture was concentrated under reduced pressure. The residue was purified by reverse flash chromatography (column, C¹⁸ silica gel; mobile phase, MeCN in water (10 mmol/L NH₄HCO₃), 50% to 70% gradient in 20 min; detector, UV 220 nm) to afford the title compound (211 mg, 42% yield) as a light yellow solid. ¹H NMR (300 MHz, Chloroform-d) δ 7.94-7.91 (m, 2H), 7.86-7.78 (m, 1H), 7.66-7.50 (m, 3H), 7.12-7.08 (m, 1H), 6.93-6.89 (m, 1H), 5.97-5.93 (m, 1H), 5.22-5.12 (m, 2H), 4.56 (d, J=5.9 Hz, 2H), 4.52-4.48 (m, 1H), 4.39-4.35 (m, 1H), 3.70-3.62 (m, 2H), 3.33-3.26 (m, 1H), 3.26-3.17 (m, 2H), 3.07 (s, 3H), 2.59-2.55 (m, 2H), 2.41-2.31 (m, 4H), 2.12-2.04 (m, 3H), 1.71-1.53 (m, 6H), 1.49 (s, 9H), 1.31-1.20 (m, 2H); LC/MS (ESI, m z): [(M+1)]+=728.3.

Step 2—tert-butyl N-[(2S,11S)-2-[[(1S)-3-carbamoyl-1-[[(4-methanesulfonylphenyl)methyl]carbamoyl]propyl]carbamoyl]-12-oxo-6-(5-oxopentyl)-1-azatricyclo[6.4.1.0\hat{ }[4,13]]trideca-4(13),5,7-trien-11-yl]carbamate. To a stirred mixture of tert-butyl N-[(2S,11S)-2-[[(1S)-3-carbamoyl-1-[[(4-methanesulfonylphenyl)methyl]carbamoyl]propyl]carbamoyl]-6-(5-hydroxypentyl)-12-oxo-1-azatricyclo[6.4.1.0\hat{ }[4,13]]trideca-4(13),5,7-trien-11-yl]carbamate (200.00 mg, 0.28 mmol) in DCM (15.00 mL) was added DMP (128.20 mg, 0.30 mmol) in portions at rt under nitrogen atmosphere and the mixture was stirred for 2 h. On completion, the resulting mixture was concentrated under reduced pressure. The residue was purified by reverse flash chromatography (column, C¹⁸ silica gel; mobile phase, MeCN in water (10 mmol/L FA), 40% to 55% gradient in 20 min; detector, UV 220 nm) to afford the title compound (60 mg, 30% yield) as a light yellow solid. ¹H NMR (300 MHz, Chloroform-d) δ 9.81-9.78 (1 m, 1H), 7.95-7.85 (m, 2H), 7.55-7.51 (m, 2H), 6.89 (d, J=7.5 Hz, 2H), 5.95-5.93 (m, 1H), 5.74-5.72 (m, 1H), 5.20-5.10 (m, 2H), 4.56 (d, J=5.9 Hz, 2H), 4.52-4.49 (m, 1H), 4.36-4.34 (m, 1H), 3.81-3.62 (m, 1H), 3.38-3.14 (m, 2H), 3.08-3.04 (m, 3H), 2.62-2.56 (m, 2H), 2.53-2.44 (m, 2H), 2.38-2.31 (m, 2H), 2.30-2.20 (m, 1H), 2.16-2.05 (m, 2H), 1.67-1.60 (m, 4H), 1.59-1.50 (m, 4H), 1.49 (s, 9H): [(M+1)]+=726.3.

### (2S)-2-[[(5S,8S,10aR)-5-[(tert-butoxycarbonyl)amino]-6-oxo-3-[2-[(1s,4s)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoic acid (Intermediate AP)

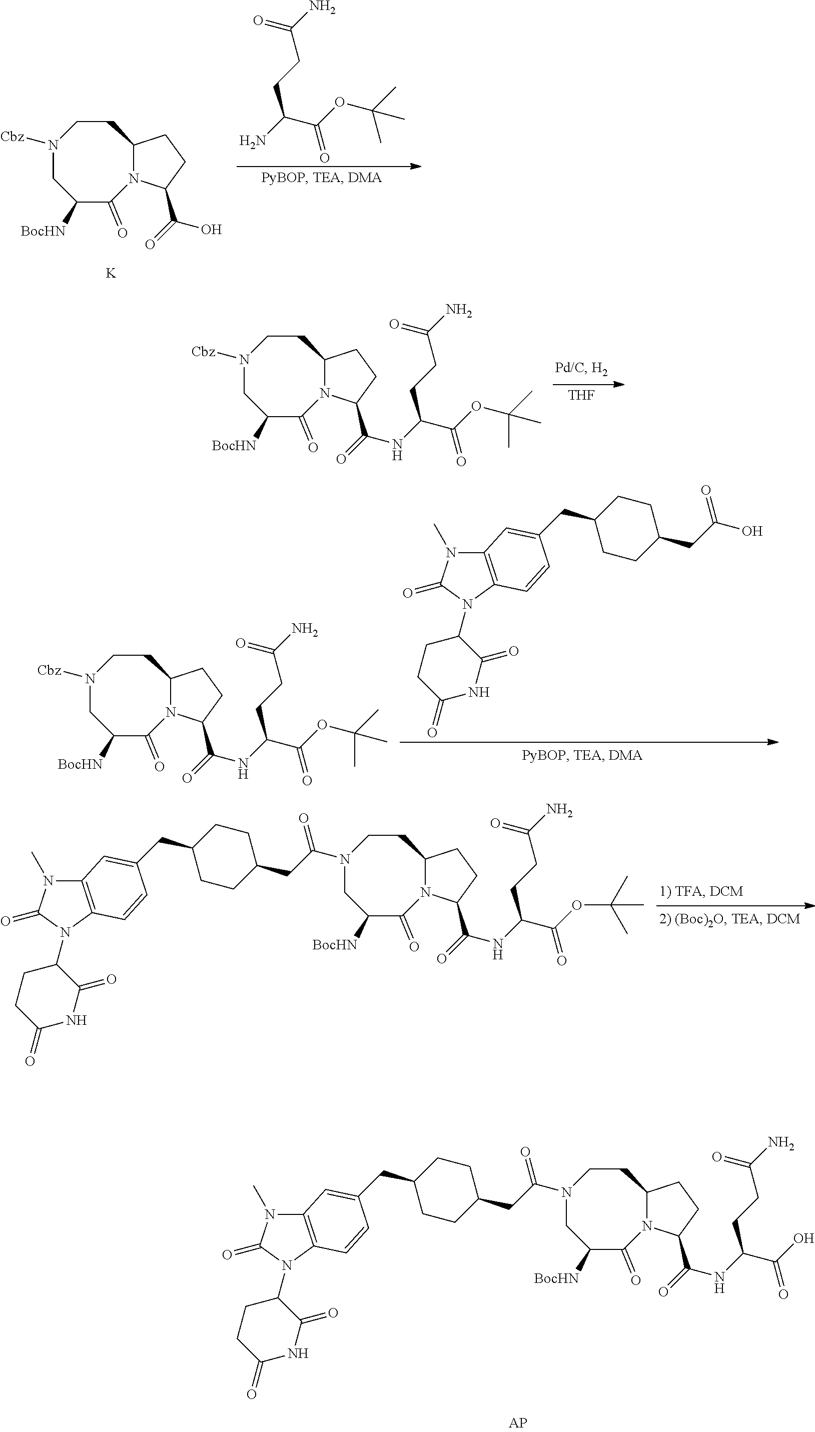

Step 1—Benzyl (5S,8S,10aR)-8-[[(2S)-1-(tert-butoxy)-4-carbamoyl-1-oxobutan-2-yl]carbamoyl]-5-[(tert-butoxycarbonyl)amino]-6-oxo-octahydropyrrolo[1,2-a][1,5]diazocine-3-carboxylate. To a stirred solution of (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (4.00 g, 8.67 mmol, Intermediate K) and glutamine t-butyl ester hydrochloride (2.28 g, 9.55 mmol) in DMA (40.00 mL) were added TEA (2.63 g, 25.99 mmol) and PyBOP (5.41 g, 10.40 mmol) at 0° C. under nitrogen atmosphere and the reaction mixture was stirred for 1 h at rt. On completion, the reaction mixture was concentrated under reduced pressure. The residue was purified by reverse phase flash chromatography (Column: WelFlash™ C18-I, 20-40 μm, 330 g; Eluent A: water (plus 10 mmol/L formic acid); Eluent B: ACN; Gradient: 40%-70% B in 30 min; Flow rate: 80 mL/min; Detector: 220 nm; desired fractions were collected at 58% B) and concentrated under reduced pressure to afford the title compound (4.8 g, 81% yield) as a white solid: ¹H NMR (400 MHz, DMSO-d₆) δ 8.28 (d, J=7.2 Hz, 1H), 7.46-7.27 (m, 5H), 7.24-7.15 (m, 1H), 7.02-6.67 (m, 2H), 5.10 (d, J=1.7 Hz, 2H), 4.48-4.23 (m, 2H), 4.23-4.04 (m, 2H), 3.78-3.72 (m, 2H), 3.54 (t, J=14.4 Hz, 2H), 3.26-3.06 (m, 2H), 2.17 (q, J=6.2, 5.0 Hz, 3H), 2.05-1.62 (m, 4H), 1.48-1.28 (m, 20H); LC/MS (ESI, m z): [(M+H)]⁺=646.3.

Step 2—Tert-butyl (2S)-2-[[(5S,8S,10aR)-5-[(tert-butoxycarbonyl)amino]-6-oxo-octahydro-1H-pyrrolo[1,2-a][1,5]diazocin-8-yl]formamido]-4-carbamoylbutanoate. To a solution of benzyl (5S,8S,10aR)-8-[[(2S)-1-(tert-butoxy)-4-carbamoyl-1-oxobutan-2-yl]carbamoyl]-5-[(tert-butoxycarbonyl)amino]-6-oxo-octahydropyrrolo[1,2-a][1,5]diazocine-3-carboxylate (4.60 g, 7.12 mmol) in THF (200.00 mL) was added Pd/C (758.08 mg, 10% palladium on activated carbon) under nitrogen atmosphere. The mixture was hydrogenated at rt for 16 h under hydrogen atmosphere using a hydrogen balloon. After the reaction was completed, it was filtered through a celite pad and concentrated under reduced pressure to give the title compound (3.7 g, 91% yield) as a white solid. ¹H NMR (400 MHz, DMSO-d₆) δ 8.84 (d, J=7.7 Hz, 1H), 7.20 (d, J=6.4 Hz, 1H), 6.81-6.68 (m, 2H), 4.63-4.54 (m, 1H), 4.42-4.25 (m, 2H), 4.13-3.98 (m, 1H), 3.64-3.55 (m, 1H), 3.15-3.05 (m, 1H), 2.88-2.63 (m, 3H), 2.34-2.06 (m, 3H), 2.06-1.87 (m, 1H), 1.87-1.64 (m, 3H), 1.40 (s, 9H), 1.37 (s, 9H), 1.35 (d, J=8.2 Hz, 3H); LC/MS (ESI, m z): [(M+H)]+=512.2.

Step 3—Tert-butyl (2S)-2-[[(5S,8S,10aR)-5-[(tert-butoxycarbonyl)amino]-6-oxo-3-[2-[(1S,4S)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoate. To a stirred solution of tert-butyl (2S)-2-[[(5S,8S,10aR)-5-[(tert-butoxycarbonyl)amino]-6-oxo-octahydro-1H-pyrrolo[1,2-a][1,5]diazocin-8-yl]formamido]-4-carbamoylbutanoate (695.00 mg, 1.36 mmol) and [(1S,4S)-4-[[1-(2,6-dioxopiperidin-3-yl)-3-methyl-2-oxo-1,3-benzodiazol-5-yl]methyl]cyclohexyl]acetic acid (561.68 mg, 1.36 mmol) in DMA (8.00 mL) were added TEA (412.38 mg, 4.08 mmol) and PyBOP (848.30 mg, 1.63 mmol) at 0° C. under nitrogen atmosphere. The resulting mixture was stirred for 1 h at room temperature under nitrogen atmosphere. On completion, the reaction mixture was concentrated under reduced pressure. The residue was purified by reverse phase flash chromatography (Column: WelFlash™ C18-I, 20-40 μm, 330 g; EluentA: water (plus 10 mmol/L formic acid); Eluent B: ACN; Gradient: 35%-55% B in 20 min; Flow rate: 80 mL/min; Detector: 220/254 nm; desired fractions were collected at 50% B) and concentrated under reduced pressure to afford the title compound (950 mg, 73% yield) as a white solid. ¹H NMR (300 MHz, DMSO-d₆) δ 11.06 (s, 1H), 8.27 (d, J=7.7 Hz, 1H), 7.19 (d, J=7.2 Hz, 1H), 7.07-6.89 (m, 2H), 6.91-6.65 (m, 2H), 6.50 (d, J=6.8 Hz, 1H), 5.33 (dd, J=12.8, 5.4 Hz, 1H), 4.40 (q, J=8.7, 7.5 Hz, 2H), 4.20-3.86 (m, 2H), 3.77 (t, J=13.8 Hz, 2H), 3.61-3.42 (m, 1H), 3.38-3.29 (s, 3H), 3.19-3.06 (m, 1H), 2.99-2.81 (m, 2H), 2.81-2.54 (m, 6H), 2.47-2.24 (m, 2H), 2.16 (q, J=5.4, 3.0 Hz, 4H), 2.06-1.87 (m, 2H), 1.86-1.56 (m, 4H), 1.44 (s, 9H), 1.39 (s, 9H), 1.37-1.33 (m, 8H); LC/MS (ESI, m z): [(M+1)]+=907.3.

Step 4—(2S)-2-[[(5S,8S,10aR)-5-[(tert-butoxycarbonyl)amino]-6-oxo-3-[2-[(1S,4S)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoic acid. To a stirred solution of tert-butyl (2S)-2-[[(5S,8S,10aR)-5-[(tert-butoxycarbonyl)amino]-6-oxo-3-[2-[(1S,4S)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoate (3.80 g, 4.19 mmol) in DCM (20.00 mL) was added TFA (4.00 mL, 53.85 mmol) at rt under nitrogen atmosphere and the mixture was stirred for 4 h. The resulting mixture was concentrated under reduced pressure then was diluted with DCM (10.00 mL). To the above mixture was added TEA (1.70 g, 16.80 mmol) dropwise and Boc₂O (1.01 g, 4.63 mmol) at 0° C. The reaction mixture was stirred for additional 16 h at rt. On completion, the reaction mixture was concentrated under reduced pressure. The residue was purified by reverse phase flash chromatography (Column: WelFlash™ C18-I, 20-40 μm, 330 g; Eluent A: water (plus 10 mmol/L formic acid); Eluent B: ACN; Gradient: 30%-50% B in 20 min; Flow rate: 80 mL/min; Detector: 220/254 nm; desired fractions were collected at 42% B) and concentrated under reduced pressure to afford the title compound (3.0 g, 80% yield) as a white solid. ¹H NMR (300 MHz, DMSO-d₆) δ 12.55 (s, 1H), 11.08 (s, 1H), 8.29 (d, J=7.7 Hz, 1H), 7.22 (s, 1H), 7.01 (dd, J=8.9, 4.1 Hz, 2H), 6.93-6.68 (m, 2H), 6.53 (d, J=6.8 Hz, 1H), 5.35 (dd, J=12.6, 5.3 Hz, 1H), 4.48-4.30 (m, 3H), 4.26-3.99 (m, 2H), 3.89-3.71 (m, 2H), 3.33 (s, 3H), 3.18-3.02 (m, 1H), 2.90 (d, J=14.9 Hz, 1H), 2.81-2.56 (m, 2H), 2.42 (d, J=9.6 Hz, 1H), 2.19 (d, J=7.9 Hz, 2H), 2.02 (d, J=13.4 Hz, 2H), 1.89-1.57 (m, 2H), 1.53-1.29 (m, 12H), 1.07 (d, J=1.1 Hz, 6H), 1.05 (s, 9H); LC/MS (ESI, m z): [(M+H)]+=851.4.

### (4-(isopropylsulfonyl)phenyl)methanamine (Intermediate AQ)

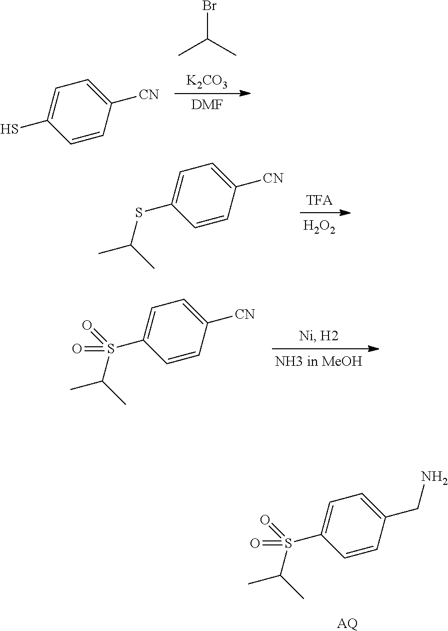

Step 1—4-(isopropylthio)benzonitrile. To a stirred mixture of 4-sulfanylbenzonitrile (10.00 g, 73.98 mmol) and 2-bromopropane (27.30 g, 221.93 mmol) in DMF (150.00 mL) was added K₂CO₃ (81.79 g, 591.80 mmol) in portions at rt. The resulting mixture was stirred for 16 h at 60° C. under nitrogen atmosphere. On completion, the mixture was cooled to rt and extracted with EtOAc (3×100 mL). The combined organic layers were washed with brine (3×200 mL), and dried over anhydrous Na₂SO₄. After filtration, the filtrate was concentrated under reduced pressure. The residue was purified by silica gel column chromatography, eluted with PE/EtOAc (5:1), to afford the title compound (12.7 g, 97% yield) as an orange oil. ¹H NMR (400 MHz, Chloroform-d) δ 7.59-7.51 (m, 2H), 7.41-7.33 (m, 2H), 3.62-3.52 (m, 1H), 1.39 (d, J=6.7 Hz, 6H); LC/MS (ESI, m/z): [(M+H)]⁺=178.2.

Step 2—4-(isopropylsulfonyl)benzonitrile. To a stirred mixture of 4-(isopropylsulfanyl)benzonitrile (5.00 g, 28.21 mmol) in TFA (100.00 mL) was added H₂O₂ (30% solution) (100 mL) dropwise at 0° C. The resulting mixture was stirred for 2 h at rt. On completion, the reaction mixture was extracted with EtOAc (3×100 mL). The combined organic layers were washed with brine (1×200 mL), and dried over anhydrous Na₂SO₄. After filtration, the filtrate was concentrated under reduced pressure to the title compound (4.8 g, 81% yield) as a white solid. ¹H NMR (400 MHz, Chloroform-d) δ 8.08-7.97 (m, 2H), 7.95-7.81 (m, 2H), 3.29-3.20 (m, 1H), 1.31 (d, J=6.9 Hz, 6H); LC/MS (ESI, m/z): [(M+H)]+=210.0.

Step 3—(4-(isopropylsulfonyl)phenyl)methanamine. To a solution of 4-(propane-2-sulfonyl)benzonitrile (4.80 g, 22.94 mmol) in 40 mL 7 M NH₃ in MeOH was added Ni (5 g) under nitrogen atmosphere in a 250 mL round-bottom flask. The mixture was hydrogenated at rt and stirred for 16 h under hydrogen atmosphere using a hydrogen balloon. After completion of reaction, the reaction mixture was filtered through a celite pad and the filter cake was washed with MeOH (2×10 mL). The filtrate was concentrated under reduced pressure. The residue was purified by reverse phase rlash chromatography (Column: WelFlash™ C18-I, 20-40 μm, 330 g; EluentA: Water; Eluent B: ACN; Gradient: 2%-20% B in 25 min; Flow rate: 80 mL/min; Detector: 220/254 nm; desired fractions were collected at 12% B) and concentrated under reduced pressure to afford the title compound (3.28 g, 67% yield) as a yellow oil. ¹H NMR (300 MHz, DMSO-d₆) δ 7.79 (d, J=8.6 Hz, 2H), 7.64 (d, J=8.0 Hz, 2H), 3.85 (s, 2H), 3.47-3.27 (m, 1H), 1.16 (dd, J=6.8, 1.2 Hz, 6H); LC/MS (ESI, m/z): [(M+H)]⁺=214.2.

### (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (Intermediate AS)

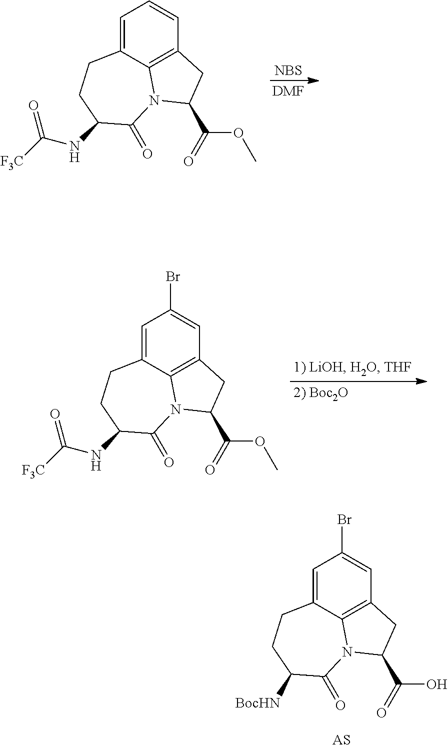

Step 1—Methyl (2S,11S)-6-bromo-12-oxo-11-(2,2,2-trifluoroacetamido)-1-azatricyclo[6.4.1.0\hat{ }[4,13]]trideca-4(13),5,7-triene-2-carboxylate. A solution of methyl (2S,11S)-12-oxo-11-(2,2,2-trifluoroacetamido)-1-azatricyclo[6.4.1.0\hat{ }[4,13]]trideca-4(13),5,7-triene-2-carboxylate (10.00 g, 28.07 mmol) and NBS (7.49 g, 42.10 mmol) in DMF (10.00 mL) was stirred for overnight at rt under nitrogen atmosphere. On completion, the solution was purified by reverse phase flash chromatography (Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 33% B-45% B gradient in 20 min; Detector: 254/220 nm; the fractions containing the desired product were collected at 40% B) and concentrated under reduced pressure to afford the title compound as a yellow solid (7.0 g, 57% yield): ¹H NMR (400 MHz, DMSO-d₆) δ 9.90 (d, J 7.9 Hz, 1H), 7.32 (dd, J=12.7, 2.0 Hz, 2H), 5.16 (dd, J=11.3, 2.9 Hz, 1H), 4.67-4.41 (m, 1H), 3.65 (s, 3H), 3.57-3.53 (m, 1H), 3.43-3.37 (m, 1H), 3.18-3.12 (m, 1H), 3.1-3.00 (m, 1H), 2.14-2.11 (m, 2H). LC/MS (ESI, m z): [(M+1)]=434.9, 436.9.

Step 2. (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid. A solution of methyl (2S,11S)-6-bromo-12-oxo-11-(2,2,2-trifluoroacetamido)-1-azatricyclo[6.4.1.0\hat{ }[4,13]]trideca-4(13),5,7-triene-2-carboxylate (7.5 g, 17.23 mmol) and aqueous LiOH (2 M, 51.70 mL, 103.40 mmol) in THF (50.00 mL) was stirred for 3 h at rt under nitrogen atmosphere. The solution was then neutralized to pH 10 with HCl (1 M). To the above solution was added Boc₂O (4.06 mL, 18.585 mmol) at rt. The resulting mixture was stirred for additional overnight at rt. On completion, the reaction mixture was purified by reverse phase flash chromatography (Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 33% B-45% B gradient in 20 min; Detector: 254/220 nm; the fractions containing the desired product were collected at 40% B) and concentrated under reduced pressure to afford the title compound as a white solid (5 g, 68% yield). ¹H NMR (400 MHz, DMSO-d₆) δ 7.27-7.19 (m, 2H), 6.95 (d, J 7.7 Hz, 1H), 4.76 (dd, J 10.5, 2.1 Hz, 1H), 3.99 (q, J 6.7 Hz, 1H), 3.29-3.26 (m, 1H), 3.11-2.91 (m, 3H), 2.02-2.00 (m, 2H), 1.39 (s, 9H). LC/MS (ESI, m z): [(M+1-100)]+=325.1, 327.1.

### tert-butyl N-[(2S,11S)-6-bromo-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamate (Intermediate AT)

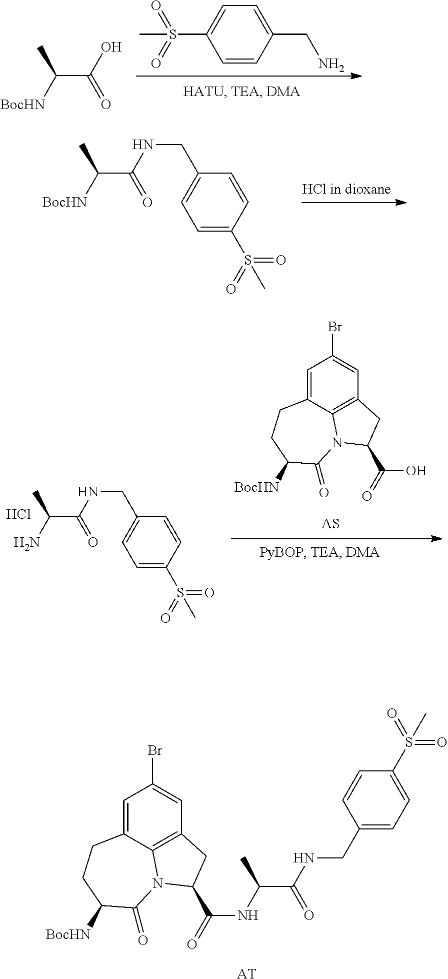

Step 1—tert-butyl N-[(1S)-1-[[(4-methanesulfonylphenyl)methyl]carbamoyl]ethyl]carbamate. To a stirred mixture of (2S)-2-[(tert-butoxycarbonyl)amino]propanoic acid (612.86 mg, 3.24 mmol) and HATU (1231.58 mg, 3.24 mmol) in DMA (5 mL) were added TEA (819.40 mg, 8.10 mmol) dropwise and 1-(4-methanesulfonylphenyl)methanamine (500.00 mg, 2.70 mmol) in portions at rt under nitrogen atmosphere and the mixture was stirred for 16. On completion, the residue was purified by reverse flash chromatography (column, C¹⁸ silica gel; mobile phase, MeCN in water (10 mmol/L NH₄HCO₃), 25% to 45% gradient in 25 min; detector, UV 254 nm) to afford the title compound (915 mg, 95% yield) as a white solid. ¹H NMR (300 MHz, Chloroform-d) δ 7.93-7.83 (m, 2H), 7.51-7.42 (m, 2H), 6.96 (s, 1H), 5.01 (s, 1H), 4.57-4.53 (m, 2H), 4.25-4.21 (m, 1H), 3.05 (s, 3H), 1.45 (s, 9H), 1.42 (d, J=7.1 Hz, 3H); LC/MS (ESI, m z): [(M+1)]+=357.1.

Step 2—(2S)-2-amino-N-[(4-methanesulfonylphenyl)methyl]propanamide hydrochloride. To a stirred solution of tert-butyl N-[(1S)-1-[[(4-methanesulfonylphenyl)methyl]carbamoyl]ethyl]carbamate (915.00 mg, 2.57 mmol) in THF (5.00 mL) was added HCl (gas) in 1,4-dioxane (5.00 mL) dropwise at rt under nitrogen atmosphere and the mixture was stirred for 2 h. On completion, the resulting mixture was concentrated under reduced pressure to afford the title compound (731 mg, 97% yield) as a light yellow solid. ¹H NMR (300 MHz, DMSO-d₆) δ 9.35 (s, 1H), 8.38 (s, 2H), 7.95-7.85 (m, 2H), 7.62-7.50 (m, 2H), 4.45 (d, J=5.9 Hz, 2H), 4.00-3.90 (m, 1H), 3.22 (s, 3H), 1.44 (d, J=6.9 Hz, 3H); LC/MS (ESI, m z): [(M+1)]+=257.2.

Step 3—tert-butyl N-[(2S,11S)-6-bromo-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamate. To a stirred mixture of (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (1274.18 mg, 3.00 mmol, Intermediate AS) and HATU (1139.21 mg, 3.00 mmol) in DMA (3.00 mL) were added TEA (757.94 mg, 7.49 mmol) dropwise and (2S)-2-amino-N-[(4-methanesulfonylphenyl)methyl]propanamide hydrochloride (731.00 mg, 2.50 mmol) in portions at rt under nitrogen atmosphere and the mixture was stirred for 16 h. On completion, the residue was purified by reverse flash chromatography (column, C¹⁸ silica gel; mobile phase, MeCN in water (10 mmol/L NH₄HCO₃), 45% to 65% gradient in 25 min; detector, UV 254 nm) to afford the title compound (1.1586 g, 70% yield) as a white solid. ¹H NMR (300 MHz, DMSO-d₆) δ 8.57-8.53 (m, 1H), 8.29 (d, J=7.2 Hz, 1H), 7.91-7.82 (m, 2H), 7.48 (d, J=8.1 Hz, 2H), 7.29 (d, J=13.4 Hz, 2H), 7.13 (d, J=8.0 Hz, 1H), 5.13-5.09 (m, 1H), 4.43-4.39 (m, 2H), 4.33-4.22 (m, 1H), 4.07-4.03 (m, 1H), 3.53-3.37 (m, 1H), 3.35-3.31 (m, 1H), 3.22 (s, 3H), 3.15-2.93 (m, 2H), 2.14-1.90 (m, 2H), 1.40 (s, 9H), 1.28 (d, J=7.1 Hz, 3H); LC/MS (ESI, m/z): [(M+1)]+=663.1, 665.1.

### 3-[5-(6-bromohexyl)-3-methyl-2-oxo-1,3-benzodiazol-1-yl]piperidine-2,6-dione (Intermediate AU)

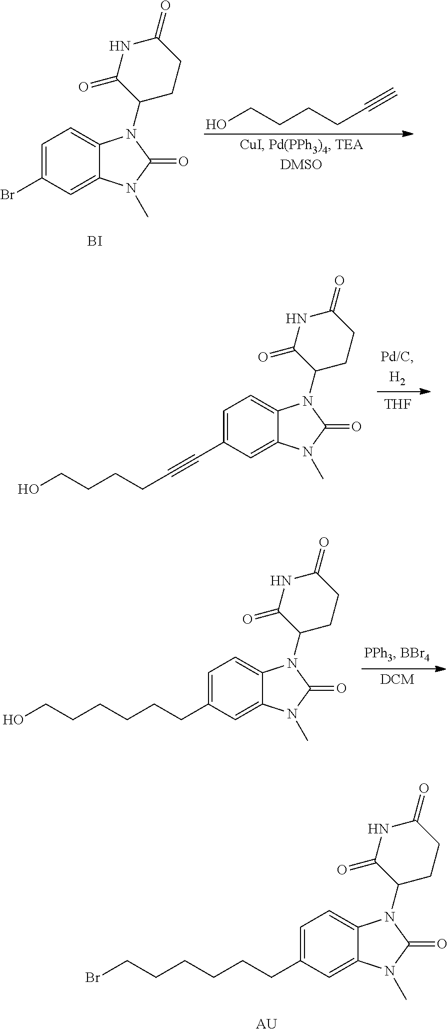

Step 1—3-[5-(6-hydroxyhex-1-yn-1-yl)-3-methyl-2-oxo-1,3-benzodiazol-1-yl]piperidine-2,6-dione. To a solution of 3-(5-bromo-3-methyl-2-oxo-1,3-benzodiazol-1-yl)piperidine-2,6-dione (4.00 g, 11.829 mmol, Intermediate BI) and hex-5-yn-1-ol (3.48 g, 35.49 mmol) in TEA (10.00 mL) and DMSO (20.00 mL) were added CuI (225.28 mg, 1.183 mmol) and Pd(PPh₃)₄ (1.37 g, 1.183 mmol) in turns under a nitrogen atmosphere at rt. The resulting mixture was stirred at 85° C. for 3 h under a nitrogen atmosphere. On completion, the reaction mixture was concentrated under reduced pressure. The residue was purified by reverse phase flash chromatography (Column: Spherical C18, 20-40 um, 330 g; Mobile Phase A: Water (plus 10 mM FA); Mobile Phase B: ACN; Flow rate: 80 mL/min; Gradient: 15% B-45% B gradient in 25 min; Detector: 254 nm; the fractions containing the desired product were collected at 30% B) and concentrated under reduced pressure to afford the title compound (3 g, 72% yield) as a light brown solid. ¹H NMR (400 MHz, DMSO-d₆) δ 11.11 (s, 1H), 7.24 (d, J=1.1 Hz, 1H), 7.11-7.07 (m, 2H), 5.38 (dd, J=12.8, 5.3 Hz, 1H), 4.43 (t, J=5.2 Hz, 1H), 3.47-2.42 (m, 2H), 3.34 (s, 3H), 3.01-2.80 (m, 1H), 2.78-2.56 (m, 2H), 2.45-2.39 (m, 2H), 2.10-1.98 (m, 1H), 1.68-1.51 (m, 4H); LC/MS (ESI, m/z): [(M+H)]⁺=356.2.

Step 2—3-[5-(6-hydroxyhexyl)-3-methyl-2-oxo-1,3-benzodiazol-1-yl]piperidine-2,6-dione. To a solution of 3-[5-(6-hydroxyhex-1-yn-1-yl)-3-methyl-2-oxo-1,3-benzodiazol-1-yl]piperidine-2,6-dione (5.00 g) in MeOH (100 mL) was added Pd/C (10 wt %, 10.00 g) under nitrogen atmosphere. The reaction system was degassed under vacuum and purged with H₂ several times, then the mixture was hydrogenated under H₂ balloon (A1 atm) at 25° C. for 3 h. After completion of the reaction, Pd/C was filtered off through celite and the filter cake was washed with MeOH (3×30 mL). The corresponding filtrate was concentrated under reduced pressure to provide the title compound (3.72 g) as a light yellow solid. ¹H NMR (300 MHz, Chloroform-d) δ 8.69 (s, 1H), 6.94-6.82 (m, 2H), 6.75-6.71 (m, 1H), 5.28-5.18 (m, 1H), 3.65 (t, J=6.5 Hz, 2H), 3.43 (s, 3H), 2.97-2.71 (m, 3H), 2.68-2.64 (m, 2H), 2.27-2.13 (m, 1H), 1.70-1.51 (m, 4H), 1.48-1.33 (m, 4H); LC/MS (ESI, m/z): [(M+H)]⁺=360.2.

Step 3—3-[5-(6-bromohexyl)-3-methyl-2-oxo-1,3-benzodiazol-1-yl]piperidine-2,6-dione. To a stirred solution of 3-[5-(6-hydroxyhexyl)-3-methyl-2-oxo-1,3-benzodiazol-1-yl]piperidine-2,6-dione (4.00 g, 11.13 mmol) and CBr₄ (7.38 g, 22.26 mmol) in DCM (40.00 mL) was added a solution of PPh₃ (4.38 g, 16.7 mmol) in DCM (40.00 mL) dropwise at 0° C. under nitrogen atmosphere. The reaction mixture was stirred for additional 30 min at 0° C., then the mixture was stirred for overnight at rt under nitrogen atmosphere. On completion, the reaction mixture was concentrated under vacuum. The residue was partitioned between water and EtOAc. The organic layer was dried over magnesium sulfate, then concentrated under reduced pressure. The residue was purified by reverse phase flash chromatography (Column: WelFlash™ C18-I, 20-40 ?m, 330 g; EluentA: Water (plus 10 mmol/L HOAc); Eluent B: ACN; Gradient: 60%-75% B in 25 min; Flow rate: 80 mL/min; Detector: 220/254 nm; desired fractions were collected at 67% B) and concentrated under reduced pressure to afford the title compound (3.5 g, 75% yield) as an off-white solid. ¹H NMR (300 MHz, Chloroform-d) δ 8.50 (s, 1H), 6.93-6.82 (m, 2H), 6.75-6.71 (m, 1H), 5.39-5.16 (m, 1H), 3.48-3.40 (m, 5H), 3.00-2.73 (m, 3H), 2.71-2.63 (m, 2H), 2.31-2.18 (m, 1H), 1.90-1.86 (m, 2H), 1.69-1.64 (m, 2H), 1.57-1.34 (m, 4H); LC/MS (ESI, m/z): [(M+H)]⁺=422.2, 424.2.

### tert-butyl N-[(2S,11S)-6-bromo-2-[[(1S)-3-carbamoyl-1-[[(4-methanesulfonylphenyl)methyl]carbamoyl]propyl]carbamoyl]-12-oxo-1-azatricyclo[6.4.1.0\hat{ }[4,13]]trideca-4(13),5,7-trien-11-yl]carbamate (Intermediate AV)

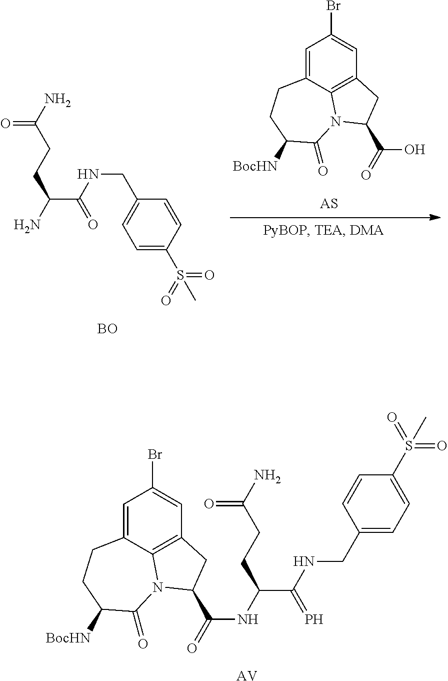

To a stirred mixture of (2S)-2-amino-N-[(4-methanesulfonylphenyl)methyl]pentanediamide (1.00 g, 3.19 mmol) and (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (1.63 g, 3.83 mmol, Intermediate AS) in DMA (15.00 mL) were added TEA (1.33 mL, 9.57 mmol) and PyBOP (1.99 g, 3.83 mmol) at rt under nitrogen atmosphere. The resulting mixture was stirred for 2 h at room temperature under nitrogen atmosphere. The reaction was monitored by LCMS. The reaction mixture was concentration under vacuum. The residue product was purified by reverse phase flash with the following conditions (Column: Spherical C18, 20˜40 um, 330 g; Mobile Phase A: Water (0.05% FA), Mobile Phase B: ACN; Flow rate: 80 mL/min; Gradient (B %): 5%˜5%, 6 min; 25%˜55%, 30 min; 95%, 5 min; Detector: 254 nm; Rt: 38 min.) to afford the title compound (1.6 g, 70% yield) as a white solid. ¹H NMR (400 MHz, DMSO-d₆) δ 8.56 (t, J=6.1 Hz, 1H), 8.30 (d, J=7.7 Hz, 1H), 7.85 (d, J=8.1 Hz, 2H), 7.48 (d, J=8.0 Hz, 2H), 7.30 (s, 1H), 7.27 (d, J=7.8 Hz, 2H), 7.13 (d, J=8.1 Hz, 1H), 6.79 (s, 1H), 5.16-5.08 (m, 1H), 4.45-4.29 (m, 2H), 4.21-4.17 (m, 1H), 4.05 (t, J=9.2 Hz, 1H), 3.49-3.38 (m, 1H), 3.19 (s, 3H), 3.11-3.02 (m, 2H), 3.01-2.93 (m, 2H), 2.15-2.06 (m, 2H), 2.05-2.00 (m, 1H), 2.00-1.72 (m, 2H), 1.39 (s, 9H); LC/MS (ESI, m z): [(M+1)]+=720.0, 722.2.

### (5S,8S,10aR)-5-[(tert-butoxycarbonyl)amino]-3-(methoxycarbonyl)-6-oxo-octahydropyrrolo[1,2-a][1,5]diazocine-8-carboxylic acid (Intermediate AW)

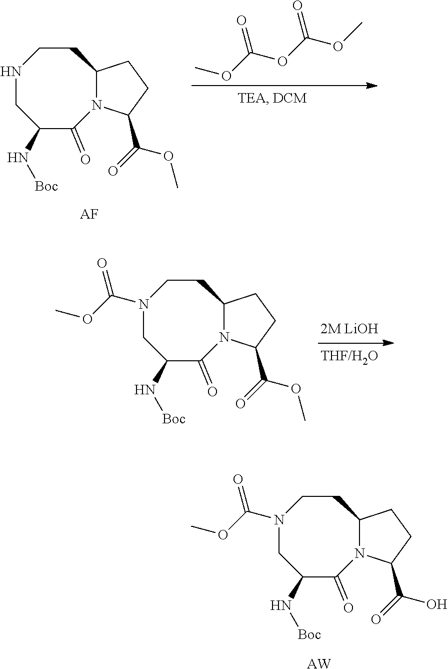

Step 1—3,8-dimethyl (5S,8S,10aR)-5-[(tert-butoxycarbonyl)amino]-6-oxo-octahydropyrrolo[1,2-a][1,5]diazocine-3,8-dicarboxylate. To a stirred solution of methyl (5S,8S,10aR)-5-[(tert-butoxycarbonyl)amino]-6-oxo-octahydro-1H-pyrrolo[1,2-a][1,5]diazocine-8-carboxylate (10.00 g, 29.29 mmol, Intermediate AF) and TEA (5.93 g, 58.58 mmol) in DCM (200 mL) was added methyl methyliumyl dicarbonate (5.85 g, 43.94 mmol) at rt under nitrogen atmosphere. The resulting mixture was stirred for 2 h at rt under nitrogen atmosphere. On completion, the resulting mixture was quenched with water (200 mL) then the reaction mixture was extracted with CH2Cl2 (2×200 mL). The combined organic layers were washed with brine (3×200 mL), and dried over anhydrous Na2SO4. After filtration, the filtrate was concentrated under reduced pressure to afford the title compound (13 g, 79% yield) as a white solid. 1H NMR (400 MHz, Chloroform-d) δ 5.60-5.56 (m, 1H), 4.59-4.54 (m, 1H), 4.50-4.44 (m, 1H), 4.23-4.16 (m, 1H), 4.08-3.80 (m, 1H), 3.75 (s, 3H), 3.73 (s, 3H), 3.64-3.37 (m, 2H), 3.31-3.24 (m, 1H), 2.37-2.31 (m, 1H), 2.15-2.07 (m, 1H), 2.05-1.89 (m, 2H), 1.89-1.78 (m, 2H), 1.42 (s, 9H). LC/MS (ESI, m/z): [(M+H)]⁺=400.2.

Step 2—(5S,8S,10aR)-5-[(tert-butoxycarbonyl)amino]-3-(methoxycarbonyl)-6-oxo-octahydropyrrolo[1,2-a][1,5]diazocine-8-carboxylic acid. To a stirred solution of 3,8-dimethyl (5S,8S,10aR)-5-[(tert-butoxycarbonyl)amino]-6-oxo-octahydropyrrolo[1,2-a][1,5]diazocine-3,8-dicarboxylate (13.00 g) in THF (30 mL) was added LiOH (3.90 g, 162.7 mmol) at rt and the mixture was stirred for 2 h at rt. On completion, the resulting mixture was concentrated under reduced pressure. The residue was purified by reverse flash chromatography (Column, Welflash™ C18-1, 20-40 m, 330 g; Eluent A: Water (plus 10 mmol/L FA); Eluent B: ACN; Gradient 20% to 50% B in 30 min; Flow rate: 80 mL/min; Detector: 220/254 nm; desired fractions were collected at 38% B) and concentrated under reduced pressure to afford the title compound (10.3 g, 82% yield) as a white solid. 1H NMR (400 MHz, Chloroform-d) δ 5.95-5.55 (m, 1H), 4.70-4.64 (m, 1H), 4.53-4.49 (m, 1H), 4.25-4.21 (m, 1H), 3.96-3.78 (m, 1H), 3.73 (s, 3H), 3.63-3.30 (m, 3H), 2.39-2.32 (m, 1H), 2.27-2.07 (m, 2H), 2.07-1.66 (m, 3H), 1.42 (s, 9H).

### 5-((diethoxyphosphoryl)difluoromethyl)benzo[b]thiophene-2-carboxylic acid (Intermediate AX)

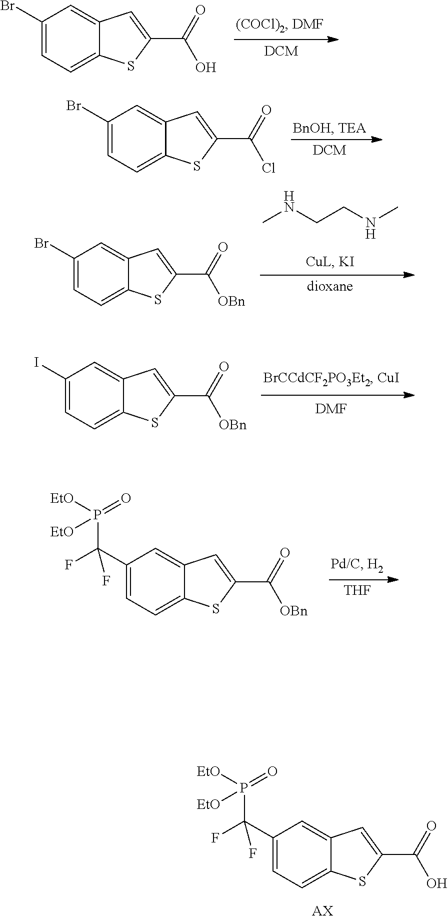

Step 1—5-bromo-1-benzothiophene-2-carbonyl chloride. To a stirred solution of 5-bromo-1-benzothiophene-2-carboxylic acid (60 g, 233 mmol, CAS #7312-10-9) in DCM (2000 mL) was added (COCl)2 (44.43 g, 350 mmol) and DMF (2 mL, 25.91 mmol) at 0° C. under nitrogen atmosphere. The resulting mixture was stirred for 2 h at rt under nitrogen atmosphere. On completion, the resulting mixture was concentrated under reduced pressure to afford the title compound (50 g, 78% yield).

Step 2—Benzyl 5-bromo-1-benzothiophene-2-carboxylate. To a stirred solution of 5-bromo-1-benzothiophene-2-carbonyl chloride (80 g, 290 mmol) and TEA (80.71 mL, 580.7 mmol) in DCM (4000 mL) was added phenylmethanol (47.09 g, 435 mmol) at 0° C. under nitrogen atmosphere. The resulting mixture was stirred for 16 h at rt under nitrogen atmosphere. On completion, the reaction mixture was quenched with sat. NH4Cl (aq.) (300 mL) at 0° C. and the mixture was extracted with CH2Cl2 (3×400 mL). The combined organic layers were washed with brine (3×500 mL), and dried over anhydrous Na2SO4. After filtration, the filtrate was concentrated under reduced pressure. The residue was purified by silica gel column chromatography, eluted with PE/EA (10:1), to afford the title compound (80 g, 79% yield) as a light yellow solid. 1H NMR (400 MHz, DMSO-d6) δ 8.49-8.41 (m, 1H), 8.22-8.15 (m, 1H), 7.96-7.88 (m, 1H), 7.85-7.79 (m, 1H), 7.51-7.46 (m, 2H), 7.45-7.37 (m, 3H), 5.44-5.33 (m, 2H); LC/MS (ESI, m/z): [(M+H)]+=347.5, 349.5.

Step 3—benzyl 5-iodo-1-benzothiophene-2-carboxylate. A 400 mL sealed bottle equipped with a magnetic stirring bar was filled with argon before adding benzyl 5-bromo-1-benzothiophene-2-carboxylate (10 g, 29 mmol), CuI (548.48 mg, 2.88 mmol), NaI (8.59 g, 57.3 mmol), methyl[2-(methylamino)ethyl]amine (2 mL, 0.576 mmol,) and dioxane (150 mL). The reaction system was charged with argon for another three times, then the mixture was stirred at 110° C. for 16 h. On completion, the reaction system was cooled to rt and quenched with ammonium chloride aqueous solution. The reaction mixture was then extracted with EtOAc (500 mL×3), washed with brine, dried with anhydrous sodium sulfate, filtered and concentrated under vacuum. The residual crude product was purified by flash column chromatography (PE:EA=10:1) to afford the title compound (9 g, 79% yield) as a white solid. 1H NMR (400 MHz, DMSO-d6) δ 8.58-8.51 (m, 1H), 8.36-8.31 (m, 1H), 7.91-7.82 (m, 1H), 7.85-7.78 (m, 1H), 7.51-7.46 (m, 2H), 7.45-7.37 (m, 3H), 5.29-5.21 (m, 2H). GC/MS (ESI, m/z): [(M)]+=394.0.

Step 4—Benzyl 5-[(diethoxyphosphoryl)difluoromethyl]-1-benzothiophene-2-carboxylate. To a solution of diethyl bromodifluoromethylphosphonate (13.48 g, 50.48 mmol) in DMF (30 mL) was added cadmium (10.01 g, 89.03 mmol) at 25° C. and the mixture was stirred at 25° C. for 4 h under N2 atmosphere. The unreacted cadmium was then removed by filtration under N2 atmosphere, and the filtrate was treated with CuCl (5.05 g, 50.99 mmol) and benzyl 5-iodo-1-benzothiophene-2-carboxylate (10.00 g, 25.37 mmol) at rt. The resulting mixture was stirred for 20 h at 25° C. under nitrogen atmosphere. On completion the reaction was quenched with sat. NH4Cl (aq.) (500 mL) at rt and the resulting mixture was extracted with EtOAc (3×800 mL). The combined organic layers were washed with brine (8×300 mL), and dried over anhydrous Na2SO4. After filtration, the filtrate was concentrated under reduced pressure. The residue was purified by reverse phase flash chromatography (Column: WelFlash™ C18-I, 20-40 ?m, 330 g; EluentA: Water (plus 10 mmol/L FA); Eluent B: ACN; Gradient: 20%-95% B in 30 min; Flow rate: 80 mL/min; Detector: 220/254 nm; desired fractions were collected at 95% B) and concentrated under reduced pressure to afford the title compound (9 g, 78% yield) as a white solid. 1H NMR (400 MHz, Chloroform-d) δ 8.21-8.14 (m, 2H), 7.99-7.92 (m, 1H), 7.76-7.70 (m, 1H), 7.51-7.47 (m, 2H), 7.46-7.35 (m, 3H), 5.45-5.39 (m, 2H), 4.30-4.11 (m, 4H), 1.39-1.28 (m, 6H); LC/MS (ESI, m/z): [(M+H)]+=455.2.

Step 5—5-[(diethoxyphosphoryl)difluoromethyl]-1-benzothiophene-2-carboxylic acid. To a solution of benzyl 5-[(diethoxyphosphoryl)difluoromethyl]-1-benzothiophene-2-carboxylate (9.00 g, 19.80 mmol) in MeOH (100 mL) was added Pd/C (716.62 mg, 6.73 mmol) under nitrogen atmosphere. The reaction system was degassed under vacuum and purged with H2 several times. Then the mixture was hydrogenated under H2 balloon (˜1 atm) at 25° C. for 16 h. After completion of the reaction, Pd/C was filtered off through celite and the filter cake was washed with MeOH (3×100 mL). The corresponding filtrate was concentrated under reduced pressure. The residue was purified by reverse phase flash chromatography (Column: WelFlash™ C18-I, 20-40 ?m, 330 g; EluentA: Water (10 mmol/L FA); Eluent B: ACN; Gradient: 20%-55% B in 30 min; Flow rate: 80 mL/min; Detector: 220/254 nm; desired fractions were collected at 48% B) and concentrated under reduced pressure to afford the title compound (5.5 g, 76% yield) as a white solid (100 mg, 49%) as a white solid. 1H NMR (300 MHz, DMSO-d6) δ 13.67 (s, 1H), 8.31-8.20 (m, 3H), 7.64-7.52 (m, 1H), 4.22-4.01 (m, 4H), 1.32-1.18 (m, 6H); LC/MS (ESI, m/z): [(M+H)]+=365.3.

### 2-(2, 3, 4, 5, 6-pentafluorophenoxycarbonyl)-1-benzothiophene-5-carbonylphosphonic acid (Intermediate AY)

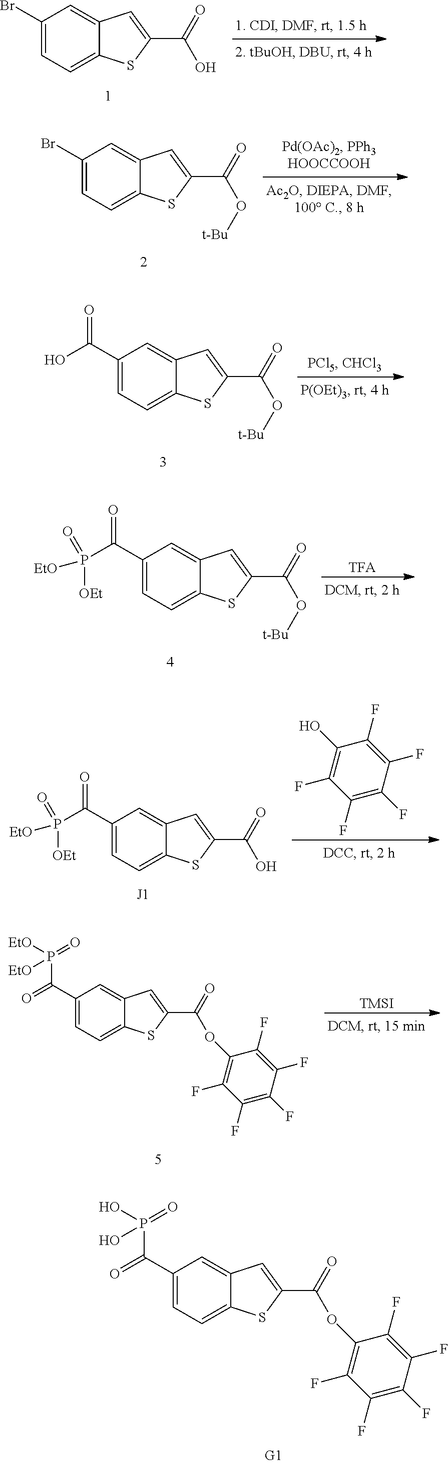

Step 1—Tert-butyl 5-bromo-1-benzothiophene-2-carboxylate. To a solution of 5-bromo-1-benzothiophene-2-carboxylic acid (25.00 g, 97.24 mmol) in DMF (200 mL) was added CDI (20.34 g, 125.44 mmol). The solution was stirred at 35° C. for 0.5 hrs. Next, t-BuOH (32.34 mL, 436.33 mmol) and DBU (17.43 mL, 114.51 mmol) was added in sequence to the reaction and the solution was stirred for another 5 hrs. On completion, the reaction was quenched with H2O and extracted with EtOAc (500 mL×3). The combined organic layers were washed with brine three times and dried with anhydrous Na2SO4. After filtration, the filter cake was concentrated under vacuum to afford the title compound (19 g, 62% yield) as a yellow solid. 1H NMR (400 MHz, Chloroform-d) δ 8.05-7.98 (m, 1H), 7.95-7.85 (m, 1H), 7.77-7.64 (m, 1H), 7.58-7.51 (m, 1H), 1.64 (s, 9H). LC/MS (ESI, m/z): [(M+H)]+=313.3, 315.3.

Step 2-2-(Tert-butoxycarbonyl)-1-benzothiophene-5-carboxylic acid. To a 400 mL sealed tube (anti-pressure) were added tert-butyl 5-bromo-1-benzothiophene-2-carboxylate (8.00 g, 25.54 mmol), Pd(AcO)2 (1.15 g, 5.11 mmol), PPh3 (4.02 g, 15.33 mmol), oxalic acid (10.06 g, 111.8 mmol), DIEA (17.80 mL, 137.7 mmol), Ac2O (9.71 mL, 95.07 mmol) and DMF (100. mL). The reaction tube was sealed and stirred at 100° C. for 8 hours. After the reaction was complete, the system was cooled to rt and quenched with 0.5 M HCl water solution until the pH was 3-5. The reaction mixture was then extracted with EtOAc (500 mL×3). The organic phase was washed with brine three times, dried with anhydrous sodium sulfate, filtered and concentrated. The residual crude product was purified by flash column chromatography (DCM:MeOH=20:1) to afford the title compound (7.5 g, 90% yield) as a yellow solid. 1H NMR (400 MHz, Chloroform-d) δ 8.67 (s, 1H), 8.19-8.14 (m, 1H), 8.11-8.06 (m, 1H), 7.99-7.94 (m, 1H), 7.77-7.72 (m, 1H), 1.65 (s, 9H). LC/MS (ESI, m/z): [(M+H)]⁺=279.1.

Step 3—Tert-butyl 5-[(diethoxyphosphoryl)carbonyl]-1-benzothiophene-2-carboxylate. To a stirred solution of 2-(tert-butoxycarbonyl)-1-benzothiophene-5-carboxylic acid (9.00 g, 32.34 mmol) in CHCl3 (100 mL) was added PCl5 (7.41 g, 35.58 mmol) in portions. The solution was stirred at room temperature for 5 minutes until the solution became clear. Then P(OEt)3 (10.75 g, 64.70 mmol) was added dropwise slowly. The solution was then stirred at rt for 4 hrs. On completion, the reaction was quenched with ice/water, then extracted with DCM (500 mL×3). The combined organic layer was washed with brine, dried with anhydrous sodium sulfate, filtered and concentrated to give the crude title product as a light yellow oil. LC/MS (ESI, m/z): [(M+H)]+=399.2.

Step 4—5-[(diethoxyphosphoryl)carbonyl]-1-benzothiophene-2-carboxylic acid. To a stirred solution of tert-butyl 5-[(diethoxyphosphoryl)carbonyl]-1-benzothiophene-2-carboxylate (15.00 g, 37.65 mmol) in DCM (150 mL) was added TFA (75 mL) dropwise at 25° C. under nitrogen atmosphere. The resulting mixture was stirred for 2 h at 25° C. under nitrogen atmosphere. On completion, the resulting mixture was concentrated under vacuum. The residue was purified by reverse phase flash chromatography (Column: WelFlash™ C18-I, 20-40 ?m, 330 g; Eluent A: Water (plus 10 mmol/L FA); Eluent B: ACN; Gradient: 20%-55% B in 30 min; Flow rate: 85 mL/min; Detector: 220/254 nm; desired fractions were collected at 48% B) and concentrated under reduced pressure to afford the title compound (1.5 g, 12% yield) as a white solid. 1HNMR (400 MHz, Chloroform-d) δ 10.34 (s, 1H), 9.08-9.01 (m, 1H), 8.35-8.30 (m, 1H), 8.23-8.19 (m, 1H), 8.03-7.96 (m, 1H), 4.45-4.29 (m, 4H), 1.55-1.35 (m, 6H); LC/MS (ESI, m/z): [(M+H)]+=343.0.

Step 5—2,3,4,5,6-pentafluorophenyl 5-[(diethoxyphosphoryl)carbonyl]-1-benzothiophene-2-carboxylate. To a stirred mixture of 5-[(diethoxyphosphoryl)carbonyl]-1-benzothiophene-2-carboxylic acid (1.00 g, 2.92 mmol) and pentafluorophenol (0.81 g, 4.38 mmol) in DCM (15 mL) was added DCC (0.90 g, 4.35 mmol) at rt under nitrogen atmosphere. The resulting mixture was stirred for 2 h at rt under nitrogen atmosphere. On completion, the resulting mixture was concentrated under reduced pressure. The residue was purified by reverse phase flash chromatography (Column: WelFlash™ C18-I, 20-40 ?m, 120 g; EluentA: Water (plus 10 mmol/L FA); Eluent B: ACN; Gradient: 25%-85% B in 30 min; Flow rate: 60 mL/min; Detector: 220/254 nm; desired fractions were collected at 80% B) and concentrated under reduced pressure to afford the title compound (1.2 g, 81% yield) as a white solid. 1H NMR (400 MHz, Chloroform-d) δ 9.14-9.08 (m, 1H), 8.52-8.47 (m, 1H), 8.36-8.31 (m, 1H), 8.10-8.03 (m, 1H), 4.40-4.26 (m, 4H), 1.48-1.41 (m, 6H); LC/MS (ESI, m/z): [(M+H)]+=508.9.

Step 6—2-(2,3,4,5,6-pentafluorophenoxycarbonyl)-1-benzothiophene-5-carbonylphosphonic acid. To a stirred solution of 2,3,4,5,6-pentafluorophenyl 5-[(diethoxyphosphoryl)carbonyl]-1-benzothiophene-2-carboxylate (1.2 g, 2.36 mmol) in DCM (25 mL) was added TMSI (2.36 g, 11.79 mmol) dropwise at 25° C. under nitrogen atmosphere. The resulting mixture was stirred for 10 min at 25° C. under nitrogen atmosphere. On completion, the resulting mixture was concentrated under reduced pressure and re-dissolved with MeCN (5 mL). To the solution was added sat. Na2SO3 (aq.) (2 mL). The generated solids was collected by filtration and dried under reduced pressure to afford the title compound (1 g, 94% yield) as a white solid. 1H NMR (400 MHz, DMSO-d6) δ 9.11-9.05 (m, 1H), 8.99-8.92 (m, 1H), 8.86-8.79 (m, 1H), 8.38-8.15 (m, 3H). LC/MS (ESI, m/z): [(M+H)]+=453.5.

### 5S,8S,10aR)-5-[(tert-butoxycarbonyl)amino]-3-(methylcarbamoyl)-6-oxo-octahydropyrrolo[1,2-a][1,5]diazocine-8-carboxylic acid (Intermediate AZ)

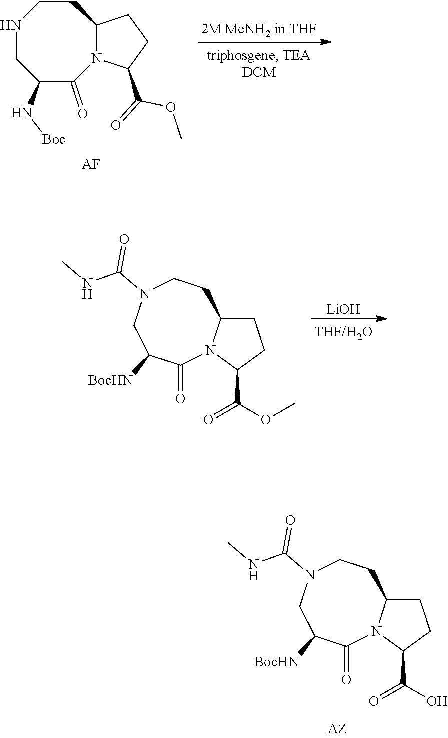

Step 1—Methyl (5S,8S,10aR)-5-[(tert-butoxycarbonyl)amino]-3-(methylcarbamoyl)-6-oxo-octahydropyrrolo[1,2-a][1,5]diazocine-8-carboxylate. To a stirred solution of triphosgene (69.54 mg, 0.234 mmol) in DCM (10 mL) was added the solution of methyl (5S,8S,10aR)-5-[(tert-butoxycarbonyl)amino]-6-oxo-octahydro-1H-pyrrolo[1,2-a][1,5]diazocine-8-carboxylate (200 mg, 0.586 mmol, Intermediate AF) and TEA (0.244 mL, 177.8 mg, 1.76 mmol) in DCM (15 mL) dropwise at rt. To the above mixture was added methylamine (292.90 uL, 0.586 mmol) and TEA (177.83 mg, 1.758 mmol) in DCM (15 mL) dropwise at rt. The resulting mixture was stirred for 2 h at rt. On completion, the residue was purified by reverse phase flash chromatography (Column: WelFlash™ C18-I, 20-40 um, 120 g; EluentA: Water (plus 10 mmol/L FA); Eluent B: ACN; Gradient: 10%-40% B in 25 min; Flow rate: 60 mL/min; Detector: 220/254 nm; desired fractions were collected at 33% B) and concentrated under reduced pressure to afford the title compound (140 mg, 60% yield) as a white solid. 1H NMR (400 MHz, Chloroform-d) δ 7.29-7.25 (m, 1H), 6.73 (s, 1H), 5.98 (s, 1H), 4.61-4.56 (m, 1H), 4.32-4.28 (m, 1H), 4.24-4.09 (m, 2H), 3.86-3.72 (m, 4H), 3.11-3.05 (m, 2H), 2.91-2.87 (m, 3H), 2.46-2.28 (m, 1H), 2.05-1.99 (m, 2H), 1.84-1.80 (m, 1H), 1.63-1.59 (m, 1H), 1.46 (s, 9H). LC/MS (ESI, m/z): [(M+H)]+=399.2.

Step 2—(5S,8S,10aR)-5-[(tert-butoxycarbonyl)amino]-3-(methylcarbamoyl)-6-oxo-octahydropyrrolo[1,2-a][1,5]diazocine-8-carboxylic acid. A solution of methyl (5S,8S,10aR)-5-[(tert-butoxycarbonyl)amino]-3-(methylcarbamoyl)-6-oxo-octahydropyrrolo[1,2-a][1,5]diazocine-8-carboxylate (130 mg, 0.326 mmol) and LiOH (78.13 mg, 3.26 mmol) in THF (2 mL)/H2O (2 mL) was stirred for 1 h at rt. On completion, the mixture was acidified pH to 4 with HCl (0.5 M.) and the mixture was extracted with EA (3×20 ml). The combined organic phase was washed with brine (10 mL), dried with Na2SO4 and filtered. The filtrate was concentrated under reduced pressure to afford the title compound (120 mg, 96% yield) as a white solid. 1H NMR (400 MHz, Chloroform-d) δ 9.30 (s, 1H), 6.81 (s, 1H), 6.00 (d, J=5.0 Hz, 1H), 4.38-4.29 (m, 1H), 4.22-4.09 (m, 2H), 3.85-3.81 (m, 1H), 3.10-3.06 (m, 1H), 3.03-2.92 (m, 1H), 2.89 (d, J=3.0 Hz, 3H), 2.47-2.43 (m, 1H), 2.24-2.11 (m, 1H), 2.12 (s, 2H), 2.06-1.95 (m, 1H), 1.87-1.83 (m, 1H), 1.66-1.54 (m, 1H), 1.47 (s, 9H). LC/MS (ESI, m/z): [(M+H)]⁺=385.2.

### Difluoro[2-(2,3,4,5,6-pentafluorophenoxycarbonyl)-1-benzothiophen-5-yl]methylphosphonic acid (Intermediate BA)

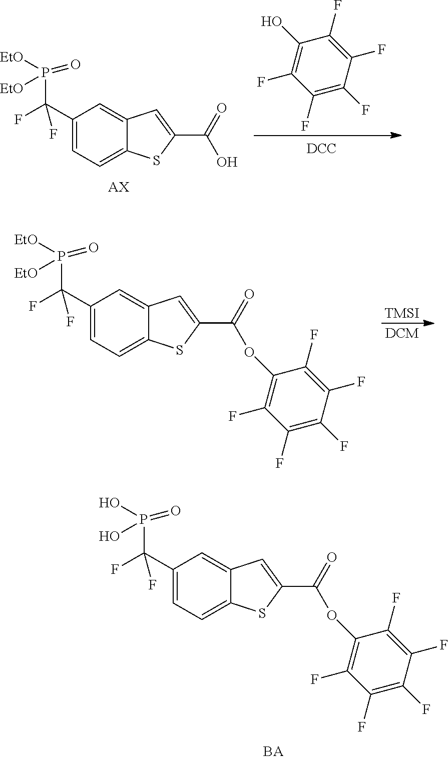

Step 1—2,3,4,5,6-pentafluorophenyl 5-[(diethoxyphosphoryl)difluoromethyl]-1-benzothiophene-2-carboxylate. To a stirred mixture of 5-[(diethoxyphosphoryl)difluoromethyl]-1-benzothiophene-2-carboxylic acid (1.0 g, 2.74 mmol, Intermediate AX) and pentafluorophenol (0.56 g, 3.04 mmol) in DCM (15 mL) was added DCC (0.85 g, 4.12 mmol) at rt under nitrogen atmosphere. The resulting mixture was stirred for 2 h at rt under nitrogen atmosphere. On completion, the resulting mixture was filtered and the filter cake was washed with DCM (3×100 mL). The filtrate was concentrated under reduced pressure and the resulting mixture was suspended in hexanes (500 mL). The precipitated solids were collected by filtration and washed with hexanes (3×50 mL) to afford the title compound (900 mg, 62% yield) as a white solid. 1H NMR (300 MHz, DMSO-d6) δ 8.35-8.28 (m, 1H), 8.26-8.20 (m, 1H), 8.15-8.09 (m, 1H), 7.78-7.64 (m, 1H), 4.21-3.99 (m, 4H), 1.35-1.21 (m, 6H). LC/MS (ESI, m/z): [(M+H)]+=531.5.

Step 2—difluoro[2-(2,3,4,5,6-pentafluorophenoxycarbonyl)-1-benzothiophen-5-yl]methylphosphonic acid as a white solid. To a stirred solution of 2,3,4,5,6-pentafluorophenyl 5-[(diethoxyphosphoryl)difluoromethyl]-1-benzothiophene-2-carboxylate (900 mg, 1.70 mmol) in DCM (15 mL) was added TMSI (1697 mg, 8.49 mmol) dropwise at 25° C. under nitrogen atmosphere. The resulting mixture was stirred for 15 min at 25° C. under nitrogen atmosphere. On completion, the resulting mixture was concentrated under reduced pressure. The residue was purified by reverse phase flash chromatography (Column: WelFlash™ C18-I, 20-40 ?m, 330 g; Eluent A: Water (plus 10 mmol/L FA); Eluent B: ACN; Gradient: 15%-75% B in 30 min; Flow rate: 90 mL/min; Detector: 220/254 nm; desired fractions were collected at 65% B) and concentrated under reduced pressure to afford the title compound (600 mg, 75% yield) as a white solid. 1H NMR (400 MHz, Methanol-d4) δ 8.61-8.55 (m, 1H), 8.38-8.31 (m, 1H), 8.21-8.14 (m, 1H), 7.88-7.81 (m, 1H). LC/MS (ESI, m/z): [(M+H)]+=475.5.

### (3-Fluoro-4-isopropylphenyl)methanamine (Intermediate BB)

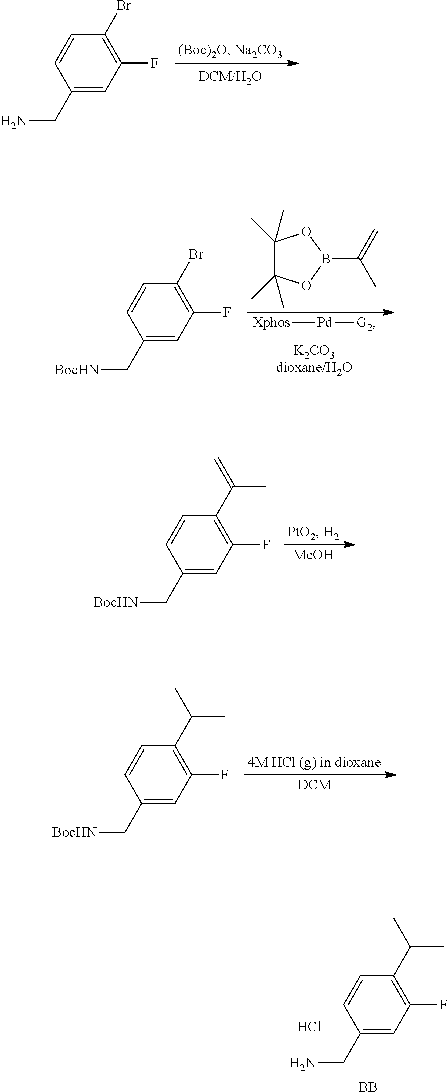

Step 1—Tert-butyl N-[(4-bromo-3-fluorophenyl) methyl] carbamate. To a stirred solution of 1-(4-bromo-3-fluorophenyl) methanamine (5.00 g, 24.50 mmol) and Na₂CO₃ (7.79 g, 73.51 mmol) in DCM (50.00 mL) and H2O (50.00 mL) was added Boc2O (5.88 g, 26.96 mmol) at 0° C. under nitrogen atmosphere. The resulting mixture was stirred for 2 h at rt under nitrogen atmosphere. On completion, the resulting mixture was diluted with water (400 mL) and the resulting mixture was extracted with CH2Cl2 (2×100 mL). The combined organic layers was washed with brine (2×100 mL), and dried over anhydrous Na2SO4. After filtration, the filtrate was concentrated under reduced pressure. The residue was purified by silica gel column chromatography (eluted with (PE/EtOAc 15:1)) to afford the title compound (3.98 g, 53% yield) as a white solid. 1H NMR (300 MHz, Chloroform-d) δ 7.49 (d, J=8.2 Hz, 1H), 7.06 (dd, J=9.3, 2.0 Hz, 1H), 6.96 (d, J=8.2 Hz, 1H), 4.27 (d, J=6.0 Hz, 2H), 1.47 (s, 9H); LC/MS (ESI, m/z): [(M−H)]−=302.0, 304.0.

Step 2—Tert-butyl N-[[3-fluoro-4-(prop-1-en-2-yl) phenyl]methyl] carbamate. To a stirred mixture of tert-butyl N-[(4-bromo-3-fluorophenyl) methyl] carbamate (3.98 g, 13.09 mmol) and 4,4,5,5-tetramethyl-2-(prop-1-en-2-yl)-1,3,2-dioxaborolane (2.64 g, 15.70 mmol) in DMA (40.00 mL) and H2O (20.00 mL) were added K2CO3 (3.62 g, 26.17 mmol) and X-Phos palladium (II) biphenyl-2-amine chloride (512.15 mg, 0.654 mmol) at rt under nitrogen atmosphere. The resulting mixture was stirred for 2 h at 80° C. under nitrogen atmosphere. On completion, the reaction mixture was allowed to cool down to rt and the mixture was concentrated under reduced pressure. The crude product was purified by reverse phase flash (Column: WelFlash™ C18-I, 20-40 m, 330 g; Eluent A: Water (plus 10 mmol/L NH4HCO3); Eluent B: ACN; Gradient: 45%-75% B in 30 min; Flow rate: 80 mL/min; Detector: 220/254 nm; desired fractions were collected at 69% B) and concentrated under reduced pressure to afford the title compound (2.68 g, 77% yield) as a pink solid. 1H NMR (400 MHz, Chloroform-d) δ 7.33-7.20 (m, 1H), 7.13-6.87 (m, 2H), 5.25-5.22 (m, 2H), 4.31 (d, J=6.1 Hz, 2H), 2.15 (s, 3H), 1.49 (s, 9H); LC/MS (ESI, m/z): [(M−H)]−=264.1.

Step 3—Tert-butyl N-[(3-fluoro-4-isopropylphenyl) methyl] carbamate. To a solution of tert-butyl N-[[3-fluoro-4-(prop-1-en-2-yl) phenyl]methyl] carbamate (2.68 g, 10.10 mmol) in MeOH (30.00 mL) was added PtO2 (350.00 mg, 1.54 mmol) under nitrogen atmosphere. The reaction system was degassed under vacuum and purged with H2 gas several times, then the mixture was hydrogenated under H2 balloon (˜1 atm) at 25° C. for 3 h. After completion of the reaction, PtO2 was filtered off through celite. The filter cake was washed with MeOH (3×10 mL). The corresponding filtrate was concentrated under reduced pressure to provide the title compound (2.53 g, 94% yield) as a colorless oil. 1H NMR (300 MHz, Chloroform-d) δ 7.24-7.12 (m, 1H), 7.02-6.88 (m, 2H), 4.26 (d, J=6.0 Hz, 2H), 3.28-3.14 (m, 1H), 1.44 (s, 9H), 1.24 (d, J=6.9 Hz, 6H); LC/MS (ESI, m/z): [(M−H)]−=266.3.

Step 4—(3-Fluoro-4-isopropylphenyl)methanamine hydrochloride. To a stirred solution of tert-butyl N-[(3-fluoro-4-isopropylphenyl) methyl] carbamate (2.53 g, 9.46 mmol) in DCM (30.00 mL) was added a solution of 4 M HCl (gas) in 1,4-dioxane (10.00 mL,) at 0° C. under nitrogen atmosphere. The resulting mixture was stirred for 2 h at rt under nitrogen atmosphere. On completion, the resulting mixture was concentrated under reduced pressure. The residue was triturated with Et2O to afford the title compound (1.9 g, 99% yield) as a white solid. 1H NMR (400 MHz, DMSO-d6) δ 8.57 (broad, 3H), 7.41-7.35 (m, 1H), 7.35-7.24 (m, 2H), 3.98 (t, J=5.7 Hz, 2H), 3.25-3.12 (m, 1H), 1.21 (d, J=6.9 Hz, 6H); LC/MS (ESI, m/z): [(M+H)]+=168.3.

### (2S)-2-Amino-N-[(3-fluoro-4-isopropylphenyl) methyl] pentanediamide (Intermediate BC)

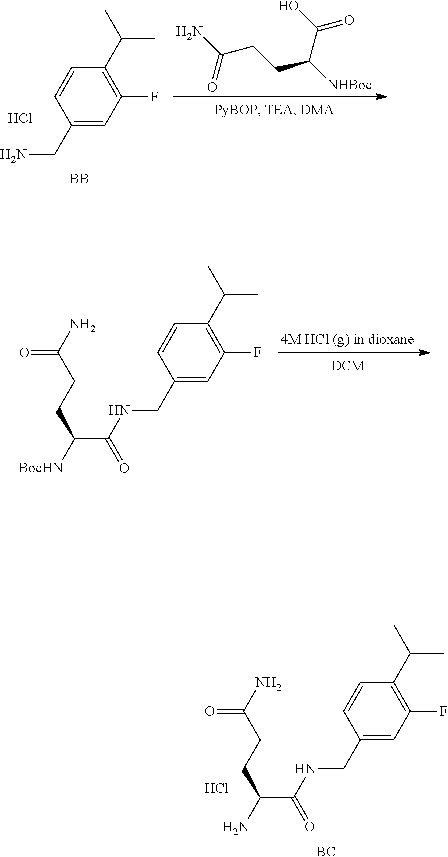

Step 1—Tert-butyl N-[(1S)-3-carbamoyl-1-[[(3-fluoro-4-isopropylphenyl) methyl]carbamoyl]propyl] carbamate. To a stirred solution of 1-(3-fluoro-4-isopropylphenyl) methanamine hydrochloride (2.01 g, 9.868 mmol, Intermediate BB) and (2S)-2-[(tert-butoxycarbonyl)amino]-4-carbamoylbutanoic acid (2.92 g, 11.84 mmol, CAS #13726-85-7) in DMA (24.00 mL) were added PyBOP (4.88 g, 12.83 mmol) and TEA (4.12 mL, 3.00 g, 29.60 mmol) at 0° C. under nitrogen atmosphere. The resulting mixture was stirred for 1 h at rt under nitrogen atmosphere. On completion, the resulting mixture was concentrated under reduced pressure. The crude product was purified by reverse phase flash chromatography (Column: WelFlash™ C18-I, 20-40 m, 330 g; Eluent A: Water (plus 10 mmol/L NH4HCO3); Eluent B: ACN; Gradient: 35%-60% B in 30 min; Flow rate: 80 mL/min; Detector: 220/254 nm; desired fractions were collected at 50% B) and concentrated under reduced pressure to afford the title compound (3.07 g, 79% yield) as a white solid. 1H NMR (400 MHz, Chloroform-d) δ 7.37 (s, 1H), 7.22-7.09 (m, 2H), 7.01-6.95 (m, 1H), 6.94-6.89 (m, 1H), 5.85-5.69 (m, 2H), 4.37 (d, J=5.4 Hz, 2H), 4.28-4.16 (m, 1H), 2.43-2.38 (m, 1H), 2.08-1.91 (m, 4H), 1.41 (s, 9H), 1.22 (d, J=6.9 Hz, 6H); LC/MS (ESI, m/z): [(M+H)]+=396.3.

Step 2—(2S)-2-Amino-N-[(3-fluoro-4-isopropylphenyl) methyl] pentanediamide hydrochloride. To a stirred mixture of tert-butyl N-[(1S)-3-carbamoyl-1-[[(3-fluoro-4-isopropylphenyl) methyl]carbamoyl]propyl] carbamate (3.07 g, 7.76 mmol) in DCM (30.00 mL) was added a solution of 4 M HCl (gas) in 1,4-dioxane (10.00 mL, 175.18 mmol) at 0° C. under nitrogen atmosphere. The resulting mixture was stirred for 2 h at rt under nitrogen atmosphere. On completion, the resulting mixture was concentrated under reduced pressure. The residue was triturated with Et₂O to afford the title compound (2.5 g, 97% yield) as a white solid. 1H NMR (300 MHz, DMSO-d6) δ 9.20 (s, 1H), 8.44 (broad, 3H), 7.53 (s, 1H), 7.35-7.19 (m, 1H), 7.14-7.02 (m, 2H), 6.93 (s, 1H), 4.44-4.20 (m, 2H), 3.94-3.81 (m, 1H), 3.22-3.07 (m, 1H), 2.30-2.16 (m, 2H), 2.07-1.91 (m, 2H), 1.20 (d, J=6.9 Hz, 6H); LC/MS (ESI, m/z): [(M+H)]+=296.3.

### (4-[3-[(2S)-2-[(tert-butoxycarbonyl)amino]-4-carbamoylbutoxy]-2-chlorophenyl]piperidin-1-yl)acetic acid (Intermediate BD)

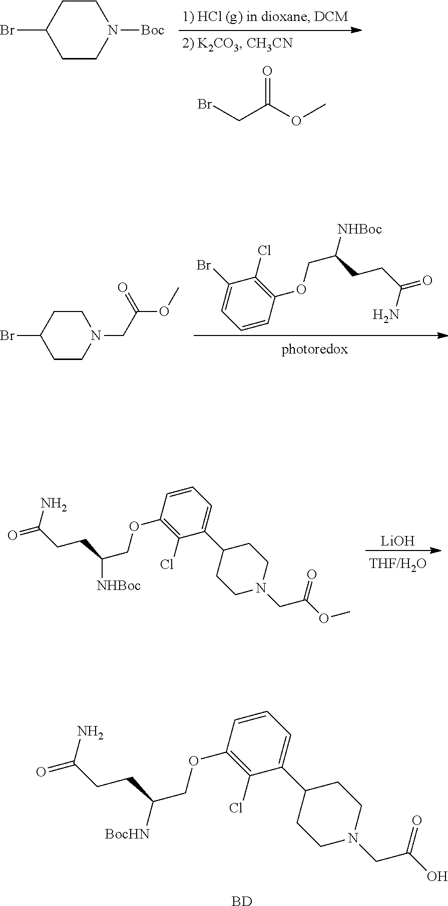

Step 1—Methyl 2-(4-bromopiperidin-1-yl)acetate. To a stirred solution of tert-butyl 4-bromopiperidine-1-carboxylate (5.50 g, 20.82 mmol) in DCM (60.00 mL) was added a solution of 4 M HCl (gas) in 1,4-dioxane (30.00 mL) dropwise at 0° C. under nitrogen atmosphere. The resulting mixture was stirred for 3 h at rt under nitrogen atmosphere. On completion, the resulting mixture was concentrated under reduced pressure. The residue was then dissolved in ACN (80.00 mL) and K2CO3 (10.01 g, 72.46 mmol) and methyl 2-bromoacetate (3.50 g, 22.88 mmol) were added and the reaction mixture was stirred for additional 16 h at rt. On completion, the mixture was concentrated under reduced pressure. The residue was purified by silica gel column chromatography (eluted with PE/EtOAc (5:1)) to afford the title compound (4.5 g, 87% yield) as a light yellow oil. 1H NMR (300 MHz, Chloroform-d) δ 4.27-4.13 (m, 1H), 3.70 (s, 3H), 3.21 (s, 2H), 2.85-2.72 (m, 2H), 2.52-2.39 (m, 2H), 2.23-2.13 (m, 2H) 2.12-1.99 (m, 2H); LC/MS (ESI, m/z): [(M+H)]+=236.1.

Step 2—methyl 2-(4-[3-[(2S)-2-[(tert-butoxycarbonyl)amino]-4-carbamoylbutoxy]-2-chlorophenyl]piperidin-1-yl)acetate. To a 20 mL vial equipped with a stir bar was added photocatalyst Ir[dF(CF3)ppy]2(dtbbpy)PF6 (15.92 mg, 0.014 mmol), methyl 2-(4-bromopiperidin-1-yl)acetate (335.00 mg, 1.42 mmol), tert-butyl N-[(2S)-1-(3-bromo-2-chlorophenoxy)-4-carbamoylbutan-2-yl]carbamate (508.60 mg, 1.21 mmol, synthesized via Steps 1-3 of Intermediate C), tris(trimethylsilyl)silane (352.81 mg, 1.42 mmol), Na2CO3 (451.14 mg, 4.26 mmol). The vial was sealed and placed under nitrogen before 10 mL of DME was added. To a separate vial was added NiCl2•glyme (3.12 mg, 0.014 mmol) and 4,4′-di-tert-butyl-2,2′-bipyridine (3.81 mg, 0.014 mmol). The catalyst vial was sealed, purged with nitrogen then to it was added 5 mL of DME. The precatalyst solution was sonicated or stirred for 5 min, after which, it was syringed into the reaction vessel. The solution was degassed by sparging with nitrogen while stirring for 10 minutes before sealing with parafilm. The reaction mixture was then stirred and irradiated with a 34 W blue LED lamp (with cooling fan to keep the reaction temperature at 25° C.) for 6 hours. The reaction was quenched by exposure to air and concentrated in vacuo. The residue was purified by reverse phase flash (Column: Spherical C18 Column, 20-40 um, 120 g; Mobile Phase A: Water (10 mmol/L NH4HCO3), Mobile Phase B: ACN; Flow rate: 60 mL/min; Gradient: 40% B to 60% B in 25 min, 254 nm, the fractions containing the desired product were collected at 50% B) to afford the title compound (300 mg, 43%) yield as a white solid. 1H NMR (300 MHz, Chloroform-d) δ 7.29 (s, 1H), 7.26-7.15 (m, 1H), 7.02-3.92 (m, 1H), 6.84-6.74 (m, 1H), 6.44-6.33 (m, 1H), 5.41-5.35 (m, 1H), 5.22-5.13 (m, 1H), 4.06 (s, 3H), 3.32 (s, 2H), 3.15-3.05 (m, 3H), 2.44-2.30 (m, 4H), 2.14-2.02 (m, 2H), 1.92-1.81 (m, 4H), 1.76-1.60 (m, 2H), 1.48 (s, 9H); LC/MS (ESI, m/z): [(M+H)]+=498.3.

Step 3—(4-[3-[(2S)-2-[(tert-butoxycarbonyl)amino]-4-carbamoylbutoxy]-2-chlorophenyl]piperidin-1-yl)acetic acid. To a stirred solution of methyl 2-(4-[3-[(2S)-2-[(tert-butoxycarbonyl)amino]-4-carbamoylbutoxy]-2-chlorophenyl]piperidin-1-yl)acetate (300.00 mg, 0.602 mmol) in THF (10.00 mL) was added LiOH·H2O (202.23 mg, 4.82 mmol) in H2O (10.00 mL) dropwise at 25° C. under nitrogen atmosphere. The resulting mixture was stirred for 2 h at rt under nitrogen atmosphere. On completion, the resulting mixture was concentrated under reduced pressure. The crude product was purified by reverse phase flash (Column: Spherical C18 Column, 20-40 um, 120 g; Mobile Phase A: Water (0.1% FA), Mobile Phase B: ACN; Flow rate: 60 mL/min; Gradient: 25% B to 50% B in 25 min, 254 nm, the fractions containing the desired product were collected at 35% B) to afford the title compound (260 mg, 89% yield) as a white solid. 1H NMR (300 MHz, Methanol-d4) δ 7.29 (t, J=8.0 Hz, 1H), 7.06-6.95 (m, 2H), 4.06-3.88 (m, 3H), 3.82-3.72 (m, 2H), 3.69 (s, 2H), 3.49-3.38 (m, 1H), 3.27-3.15 (m, 2H), 2.43-2.29 (m, 2H), 2.13-1.99 (m, 5H), 1.91-1.75 (m, 1H), 1.46 (s, 9H); LC/MS (ESI, m/z): [(M+H)]⁺=484.3.

### 6-[3-[(2S)-2-[(tert-butoxycarbonyl)amino]-4-carbamoylbutoxy]-2-fluorophenyl]hexanoic acid (Intermediate BE)

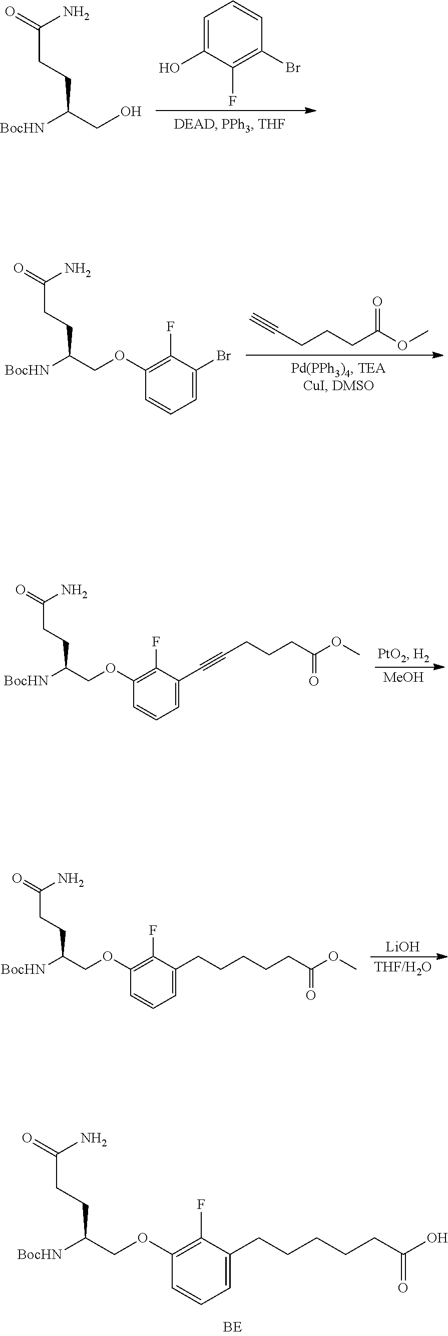

Step 1—Tert-butyl N-[(2S)-1-(3-bromo-2-fluorophenoxy)-4-carbamoylbutan-2-yl]carbamate. To a stirred solution of tert-butyl N-[(2S)-4-carbamoyl-1-hydroxybutan-2-yl]carbamate (10.00 g, 43.05 mmol, CAS #133565-42-1) and 3-bromo-2-fluorophenol (12.33 g, 64.56 mmol) in THF (100 mL) was added PPh3 (22.58 g, 86.10 mmol) in portions at 0° C. under nitrogen atmosphere. To the above mixture was added DEAD (15.00 g, 86.10 mmol) dropwise over 30 min at 0° C. The resulting mixture was stirred for additional 16 h at 25° C. under nitrogen atmosphere. On completion, the resulting mixture was concentrated under reduced pressure. The crude product was purified by silica gel column chromatography (Mobile Phase A: Petroleum ether, Mobile Phase B: EtOAc; Gradient: 0% B to 100% B in 180 min, 254 nm; the fractions containing the desired product were collected at 100% B) to afford the title compound (3.5 g, 20% yield) as a white solid. 1H NMR (300 MHz, Methanol-d4) δ 7.18-7.13 (m, 2H), 7.04-7.00 (m, 1H), 4.05 (d, J=5.5 Hz, 2H), 3.93-3.90 (m, 1H), 2.40-2.30 (m, 2H), 2.02 (d, J=5.8 Hz, 1H), 1.90-1.68 (m, 1H), 1.46 (s, 9H); LC/MS (ESI, m/z): [(M+1)]+=405.2, 407.2.

Step 2—Methyl 6-[3-[(2S)-2-[(tert-butoxycarbonyl)amino]-4-carbamoylbutoxy]-2-fluorophenyl]hex-5-ynoate. To a stirred solution of tert-butyl N-[(2S)-1-(3-bromo-2-fluorophenoxy)-4-carbamoylbutan-2-yl]carbamate (1.00 g, 2.47 mmol) and methyl hex-5-ynoate (0.93 g, 7.37 mmol, 77758-51-1) in DMSO (10 mL) was added Pd(PPh3)4 (0.29 g, 0.25 mmol), CuI (0.05 g, 0.25 mmol) and TEA (5.00 mL) at 25° C. under nitrogen atmosphere. The resulting mixture was stirred for 2 h at 80° C. under nitrogen atmosphere. On completion, the reaction mixture was purified by reverse phase flash (Column: Spherical C18 Column, 20-40 um, 120 g; Mobile Phase A: Water (5 mmol/L NH4HCO3), Mobile Phase B: ACN; Flow rate: 60 mL/min; Gradient: 40% B to 60% B in 25 min, 254 nm; the fractions containing the desired product were collected at 50% B) to afford the title compound (960 mg, 86% yield) as a colorless oil. 1H NMR (400 MHz, Methanol-d4) δ 7.11-6.94 (m, 3H), 4.05-4.01 (m, 2H), 3.95-3.85 (m, 1H), 3.72-3.67 (m, 3H), 2.57-2.53 (m, 4H), 2.37-2.33 (m, 2H), 2.05-2.01 (m, 1H), 1.96-1.92 (m, 2H), 1.83-1.81 (m, 1H), 1.46 (s, 9H); LC/MS (ESI, m/z): [(M+1)]+=451.3.

Step 3—Methyl 6-[3-[(2S)-2-[(tert-butoxycarbonyl)amino]-4-carbamoylbutoxy]-2-fluorophenyl]hexanoate. To a stirred solution of methyl 6-[3-[(2S)-2-[(tert-butoxycarbonyl)amino]-4-carbamoylbutoxy]-2-fluorophenyl]hex-5-ynoate (960.00 mg, 2.13 mmol) in MeOH (15.00 mL) was added Pd/C (100.00 mg, 0.94 mmol) at 25° C. under nitrogen atmosphere. The resulting mixture was stirred for 2 h at 25° C. under hydrogen atmosphere. On completion, the precipitated solids were collected by filtration and washed with DCM (3×30 mL) to give the crude title product (900 mg, 93% yield). 1H NMR (400 MHz, Methanol-d4) δ 7.02-6.91 (m, 2H), 6.83-6.80 (m, 1H), 4.03-4.00 (m, 2H), 3.92-3.89 (m, 1H), 3.66 (s, 3H), 2.68-2.62 (m, 2H), 2.38-2.30 (m, 4H), 2.09-1.99 (m, 1H), 1.85-1.82 (m, 1H), 1.68-1.62 (m, 4H), 1.46 (s, 9H), 1.37 (m, 2H). LC/MS (ESI, m/z): [(M+1)]⁺=455.3.

Step 4—6-[3-[(2S)-2-[(tert-butoxycarbonyl)amino]-4-carbamoylbutoxy]-2-fluorophenyl]hexanoic acid. To a stirred solution of methyl 6-[3-[(2S)-2-[(tert-butoxycarbonyl)amino]-4-carbamoylbutoxy]-2-fluorophenyl]hexanoate (900.00 mg, 1.98 mmol) in THF (10.00 mL) was added a solution of LiOH (379.34 mg, 15.84 mmol) in H2O (10.00 mL) dropwise at 25° C. under nitrogen atmosphere. The resulting mixture was stirred for 16 h at 25° C. under nitrogen atmosphere. On completion, the mixture was acidified to pH 6 with HCl (1 moL) and the resulting mixture was concentrated under reduced pressure. The crude product was purified by reverse phase flash (Column: Spherical C18 Column, 20-40 um, 120 g; Mobile Phase A: Water (0.1% FA), Mobile Phase B: ACN; Flow rate: 60 mL/min; Gradient: 30% B to 50% B in 25 min, 254 nm; the fractions containing the desired product were collected at 40% B) to afford the title compound (820 mg, 94% yield) as a colorless oil. 1H NMR (400 MHz, Methanol-d4) δ 7.02-6.90 (m, 2H), 6.84-6.79 (m, 1H), 4.05-3.98 (m, 2H), 3.94-3.86 (m, 1H), 2.67-2.64 (m, 2H), 2.39-2.27 (m, 4H), 2.09-1.99 (m, 1H), 1.85-1.82 (m, 1H), 1.68-1.62 (m, 4H), 1.46 (s, 9H), 1.43-1.35 (m, 2H); LC/MS (ESI, m/z): [(M+1)]+=441.3.

### (2S,4R)-1-[(2S)-2-(6-[3-[(2S)-2-Amino-4-carbamoylbutoxy]-2-fluorophenyl]hexanamido)-3,3-dimethylbutanoyl]-4-hydroxy-N-[(1S)-1-[4-(4-methyl-1,3-thiazol-5-yl) phenyl]ethyl] pyrrolidine-2-carboxamide (Intermediate BF)

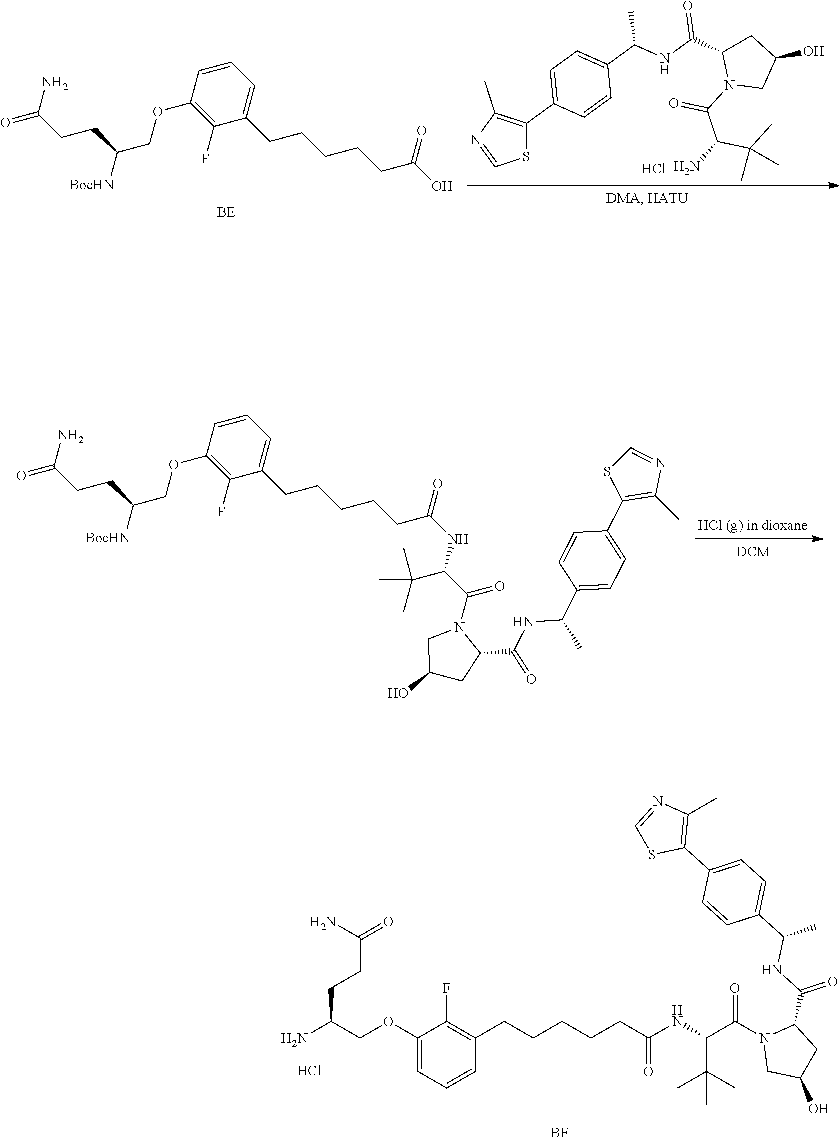

Step 1—Tert-butyl N-[(2S)-4-carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3, 3-dimethyl-1-oxobutan-2-yl] carbamoyl] pentyl) phenoxy] butan-2-yl] carbamate. To a stirred mixture of (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (203.87 mg, 0.424 mmol, CAS #1448189-80-7) and 6-[3-[(2S)-2-[(tert-butoxycarbonyl)amino]-4-carbamoylbutoxy]-2-fluorophenyl]hexanoic acid (177.80 mg, 0.404 mmol, Intermediate BE) in DMA (8.00 mL) were added PyBOP (252.05 mg, 0.484 mmol) and TEA (122.53 mg, 1.211 mmol) at rt under nitrogen atmosphere. The resulting mixture was stirred for 1 h at rt under nitrogen atmosphere. On completion, the resulting was concentrated under reduced pressure. The residue was purified by reverse phase flash chromatography (Column: WelFlash™ C18-I, 20-40 m, 120 g; EluentA: Water (plus 10 mmol/L FA); Eluent B: ACN; Gradient: 30%-60% B in 25 min; Flow rate: 60 mL/min; Detector: 220/254 nm; desired fractions were collected at 50% B) and concentrated under reduced pressure to afford the title compound (300 mg, 86% yield) as a yellow solid. 1H NMR (300 MHz, Chloroform-d) δ 8.76 (s, 1H), 7.51-7.45 (m, 1H), 7.45-7.36 (m, 4H), 6.99-6.92 (m, 1H), 6.84-6.74 (m, 2H), 6.36-6.27 (m, 2H), 5.75-5.66 (m, 1H), 5.17-5.02 (m, 2H), 4.79-4.72 (m, 1H), 4.60-4.52 (m, 1H), 4.19-4.12 (m, 1H), 4.09-3.95 (m, 2H), 3.65-3.55 (m, 1H), 3.28-3.17 (m, 4H), 2.69-2.61 (m, 1H), 2.56 (s, 3H), 2.40-2.33 (m, 1H), 2.25-2.19 (m, 1H), 2.15-1.97 (m, 1H), 1.97-1.79 (m, 4H), 1.70-1.58 (m, 4H), 1.48 (d, J=7.3 Hz, 3H), 1.46 (s, 9H), 1.39-1.21 (m, 2H), 1.05 (s, 9H); LC/MS (ESI, m/z): [(M+H)]+=867.4.

Step 2—(2S,4R)-1-[(2S)-2-(6-[3-[(2S)-2-Amino-4-carbamoylbutoxy]-2-fluorophenyl]hexanamido)-3,3-dimethylbutanoyl]-4-hydroxy-N-[(1S)-1-[4-(4-methyl-1,3-thiazol-5-yl) phenyl] ethyl]pyrrolidine-2-carboxamide hydrochloride. To a stirred mixture of tert-butyl N-[(2S)-4-carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl]carbamoyl] pyrrolidin-1-yl]-3,3-dimethyl-1-oxobutan-2-yl] carbamoyl] pentyl) phenoxy] butan-2-yl]carbamate (200.00 mg, 0.231 mmol) in DCM (4.00 mL) was added a solution of 4 M HCl (gas) in 1,4-dioxane (1.50 mL) dropwise at rt under nitrogen atmosphere. The resulting mixture was stirred for 1 h at rt. On completion, the resulting mixture was concentrated under reduced. The residue was triturated with Et2O to afford the title compound (180 mg, 97% yield) as a white solid. 1H NMR (300 MHz, DMSO-d6) δ 9.04 (s, 1H), 8.39 (d, J=7.7 Hz, 1H), 8.28 (broad, 3H), 7.80 (d, J=9.2 Hz, 1H), 7.48-7.39 (m, 5H), 7.11-7.04 (m, 2H), 6.99-6.84 (m, 2H), 4.98-4.90 (m, 1H), 4.56-4.37 (m, 2H), 4.34-4.07 (m, 3H), 3.46-3.38 (m, 1H), 3.10-3.01 (m, 4H), 2.63-2.57 (m, 1H), 2.47 (s, 3H), 2.38-2.20 (m, 2H), 2.19-1.86 (m, 2H), 1.86-1.70 (m, 4H), 1.65-1.45 (m, 4H), 1.39 (d, J=6.9 Hz, 3H), 1.32-1.24 (m, 1H), 1.15-1.08 (m, 1H), 0.94 (s, 9H); LC/MS (ESI, m/z): [(M+H)]+=767.4.

### [1-[(4-Methoxyphenyl) methyl]-2,6-dioxo-3-piperidyl] trifluoromethanesulfonate (Intermediate BG)

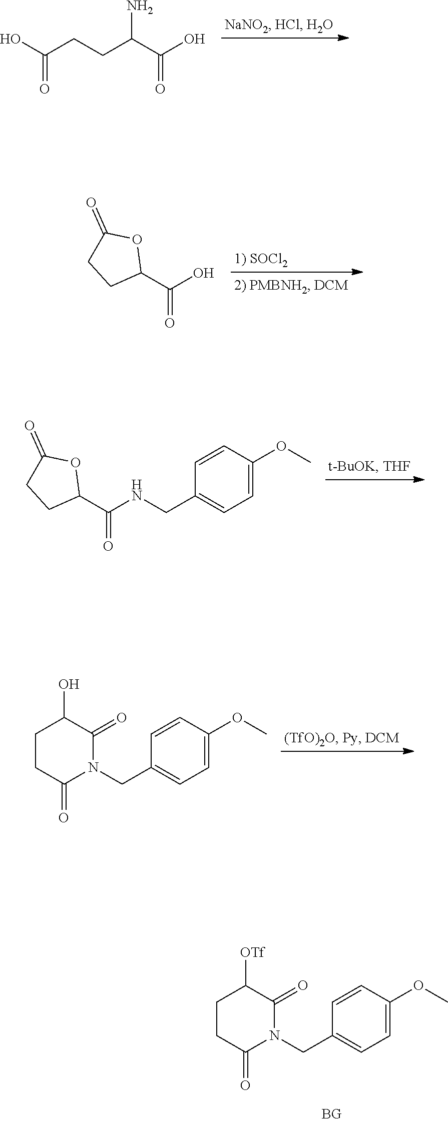 [SMILES: NC(CCC(=O)O)C(=O)O ; O=C1CCC(C(=O)O)O1 ; COc1ccc(CNC(=O)C2CCC(=O)O2)cc1 ; COc1ccc(CN2C(=O)CCC(O)C2=O)cc1 ; COc1ccc(CN2C(=O)CCC(OS(=O)(=O)C(F)(F)F)C2=O)cc1]

Step 1—5-Oxotetrahydrofuran-2-carboxylic acid. To a solution of 2-aminopentanedioic acid (210 g, 1.43 mol, CAS #617-65-2) in H2O (800 mL) and HCl (12 M, 210 mL) was added a solution of NaNO2 (147 g, 2.13 mol) in H2O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na2SO4, filtered and concentrated in vacuo to give the title compound (200 g, crude) as yellow oil. 1H NMR (400 MHz, CDCl3) δ 6.43 (s, 1H), 5.02-4.95 (m, 1H), 2.67-2.38 (m, 4H)

Step 2—N-[(4-methoxyphenyl)methyl]-5-oxo-tetrahydrofuran-2-carboxamide. To 5-oxotetrahydrofuran-2-carboxylic acid (120 g, 922 mmol) was added SOCl2 (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N2. After that a solution of Et3N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. 1H NMR (400 MHz, CDCl3) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI+) m/z 272.0 (M+Na)+.

Step 3—3-Hydroxy-1-[(4-methoxyphenyl)methyl]piperidine-2,6-dione. A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH4Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. 1H NMR (400 MHz, CDCl3) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

Step 4—[1-[(4-Methoxyphenyl) methyl]-2,6-dioxo-3-piperidyl] trifluoromethanesulfonate. To a solution of 3-hydroxy-1-[(4-methoxyphenyl) methyl] piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N2. On completion, the mixture was concentrated in vacuo. The residue was purified by column chromatography on silica gel (PE:EA=20:1/8:1) to give the title compound (45.0 g, 68% yield) as light yellow gum. 1H NMR (400 MHz, CDCl3) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 5-Bromo-3-methyl-1H-benzimidazol-2-one (Intermediate BH)

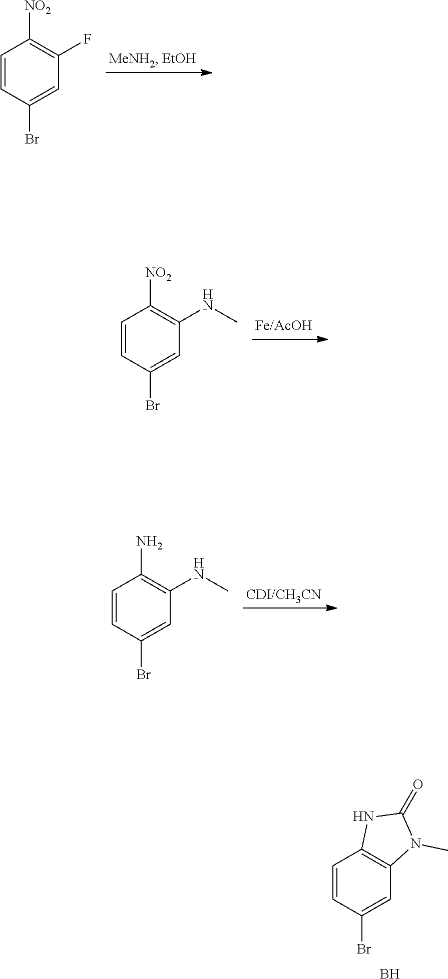

Step 1—5-Bromo-N-methyl-2-nitro-aniline. 4-bromo-2-fluoro-1-nitro-benzene (230 g, 1.05 mol, CAS #321-23-3) was added to a solution of mehylamine in tetrahydrofuran (2 M, 1.51 L). The mixture was stirred at 15° C. for 10 minutes. On completion, the mixture was diluted with H2O (250 mL) and extracted with EtOAc (3×300 mL). The combined organic layers were washed with brine (300 mL), dried over Na2SO4, filtered and concentrated in vacuo to give the title compound (200 g, 83% yield) as a yellow solid. ¹H NMR (400 MHz, DMSO-d6) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

Step 2—4-Bromo-N2-methyl-benzene-1,2-diamine. To a mixture of 5-bromo-N-methyl-2-nitro-aniline (200 g, 865 mmol) in EtOAc (1 L) and H2O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H2O (250 mL) and extracted with EtOAc (3×300 mL). The combined organic layers were washed with aq.NaHCO₃ and brine (300 mL), dried over Na2SO4, filtered and concentrated in vacuo. The residue was purified by flash silica gel chromatography to give the title compound (130 g, 75% yield) as black oil. ¹H NMR (400 MHz, DMSO-d6) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

Step 3—5-Bromo-3-methyl-1H-benzimidazol-2-one. To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N2. On completion, the mixture was concentrated in vacuo. The mixture was diluted with H2O (1.0 L) and filtered. The filter cake was washed with water (3×200 mL) and dried in vacuo to give the title compound (106 g, 85% yield) as a white solid. ¹H NMR (400 MHz, DMSO-d6) δ 11.00 (s, 1H), 7.33 (s, 1H), 7.13 (d, J=8.0 Hz, 1H), 6.92 (d, J=8.0 Hz, 1H), 3.27 (s, 3H).

### 3-(5-bromo-3-methyl-2-oxo-benzimidazol-1-yl)piperidine-2,6-dione (3-(5-bromo-3-methyl-2-oxo-2,3-dihydro-1H-1,3-benzodiazol-1-yl)piperidine-2,6-dione) (Intermediate BI)

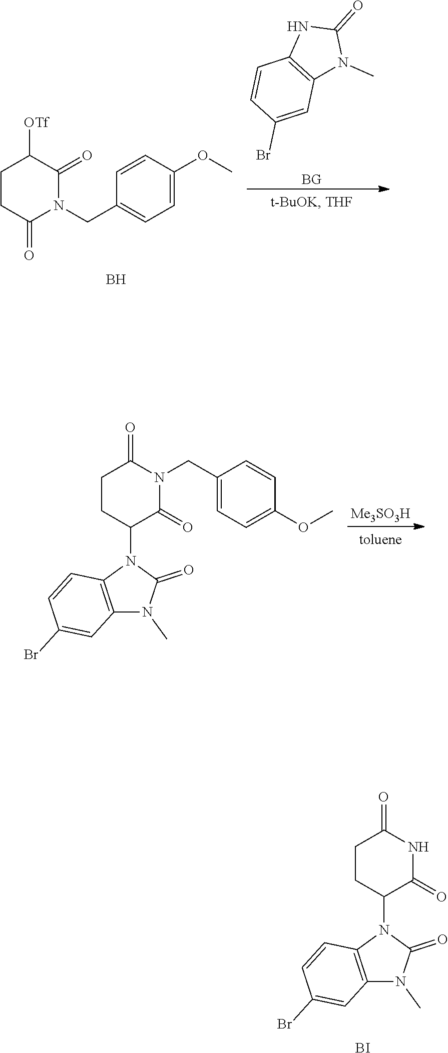

Step 1—3-(5-Bromo-3-methyl-2-oxo-benzimidazol-1-yl)-1-[(4-methoxyphenyl)methyl]piperidine-2,6-dione. To a solution of 5-bromo-3-methyl-1H-benzimidazol-2-one (4.90 g, 21.6 mmol, Intermediate BG) in THF (300 mL) was added t-BuOK (3.63 g, 32.3 mmol) at 0° C. The mixture was stirred at 0-10° C. for 1 hour under N2. Then a solution of [1-[(4-methoxyphenyl) methyl]-2, 6-dioxo-3-piperidyl] trifluoromethanesulfonate (9.87 g, 25.9 mmol, Intermediate BH) in THF (100 mL) was added to the reaction mixture at 0-10° C. during 30 minutes. The mixture was stirred at 0-10° C. for 30 minutes under N2. An additional solution of [1-[(4-methoxyphenyl) methyl]-2, 6-dioxo-3-piperidyl]trifluoromethanesulfonate (2.47 g, 6.47 mmol) in THF (20 mL) was added to the reaction mixture at 0-10° C. dropwise. The mixture was then stirred at 0-10° C. for another 30 minutes under N2. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. 1H NMR (400 MHz, DMSO-d6) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

Step 2—3-(5-Bromo-3-methyl-2-oxo-benzimidazol-1-yl)piperidine-2,6-dione. To a mixture of 3-(5-bromo-3-methyl-2-oxo-benzimidazol-1-yl)-1-[(4-methoxyphenyl)methyl] piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na2SO4, filtered and concentrated in vacuo. The residue was triturated with EA (80 mL) and filtered. The filtrate cake was collected and dried in vacuo to give the title compound (4.20 g, 67% yield) as off-white solid. 1H NMR (400 MHz, DMSO-d6) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### (2S)-2-amino-N-[(4-isopropylphenyl)methyl]pentanediamide (Intermediate BJ)

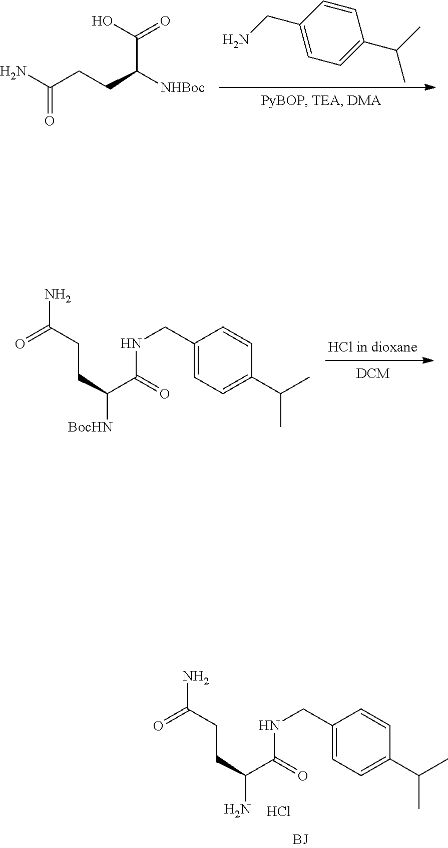 [SMILES: CC(C)(C)OC(=O)N[C@@H](CCC(N)=O)C(=O)O ; CC(C)c1ccc(CN)cc1 ; CC(C)c1ccc(CNC(=O)[C@H](CCC(N)=O)NC(=O)OC(C)(C)C)cc1 ; CC(C)c1ccc(CNC(=O)[C@@H](N)CCC(N)=O)cc1 ; Cl]

Step 1—tert-butyl N-[(1S)-3-carbamoyl-1-[[(4-isopropylphenyl)methyl]carbamoyl]propyl]carbamate. To a stirred solution of (2S)-2-[(tert-butoxycarbonyl)amino]-4-carbamoylbutanoic acid (8.00 g, 32.49 mmol) and 1-(4-isopropylphenyl)methanamine (7.27 g, 48.7 mmol) in DMA (50.00 mL) was added TEA (13.54 mL, 97.46 mmol) at rt under nitrogen atmosphere. Then, PyBOP (25.36 g, 48.73 mmol) was added at 0° C. under nitrogen atmosphere. The resulting mixture was stirred for 1 h at rt under nitrogen atmosphere. On completion, the reaction mixture was purified directly by reverse phase flash chromatography (Column: Spherical C18, 20-40 um, 330 g; Mobile Phase A: Water (plus 10 mM NH4HCO3); Mobile Phase B: ACN; Flow rate: 80 mL/min; Gradient: 5%-5% B, 8 min, 35% B-55% B gradient in 20 min; Detector: 254 nm; the fractions containing the desired product were collected at 48% B) and concentrated under reduced pressure to afford the title compound (11.8 g, 96% yield) as a white solid. ¹H NMR (400 MHz, Methanol-d4) δ 7.23-7.18 (m, 4H), 4.41-4.33 (m, 2H), 4.12-4.03 (m, 1H), 2.97-2.85 (m, 1H), 2.32 (t, J=7.7 Hz, 2H), 2.08-2.04 (m, 1H), 1.91-1.86 (m, 1H), 1.45 (s, 9H), 1.24 (d, J=7.0 Hz, 6H); LC/MS (ESI, m/z): [(M+1)]+=378.3.

Step 2—(2S)-2-amino-N-[(4-isopropylphenyl)methyl]pentanediamide hydrochloride. To a stirred solution of tert-butyl N-[(1S)-3-carbamoyl-1-[[(4-isopropylphenyl)methyl]carbamoyl]propyl]carbamate (11.80 g, 31.26 mmol) in DCM (100.00 mL) was added 4 M HCl (gas) in 1,4-dioxane (40.00 mL) dropwise at rt under nitrogen atmosphere. The resulting mixture was stirred for 1 h at rt under nitrogen atmosphere. On completion, the reaction mixture was concentrated under vacuum. The residue was triturated with Et2O. The solid was collected by filtration and dried over vacuum to afford the title compound (13.0 g) as a white solid. ¹H NMR (300 MHz, DMSO-d6) δ 8.95 (t, J=5.7 Hz, 1H), 8.28 (broad, 3H), 7.47-7.44 (m, 1H), 7.25-7.19 (m, 4H), 6.92 (s, 1H), 4.39-4.18 (m, 2H), 3.80 (t, J=6.3 Hz, 1H), 2.88-2.83 (m, 1H), 2.19 (dd, J=9.1, 6.1 Hz, 2H), 1.97-1.93 (m, 2H), 1.18 (d, J=6.9 Hz, 6H); LC/MS (ESI, m/z): [(M+1)]+=278.3.

### (1S,4S)-4-[2-[1-(2,6-dioxopiperidin-3-yl)-3-methyl-2-oxo-1,3-benzodiazol-5-yl]ethyl]cyclohexane-1-carboxylic acid (Intermediate BK)

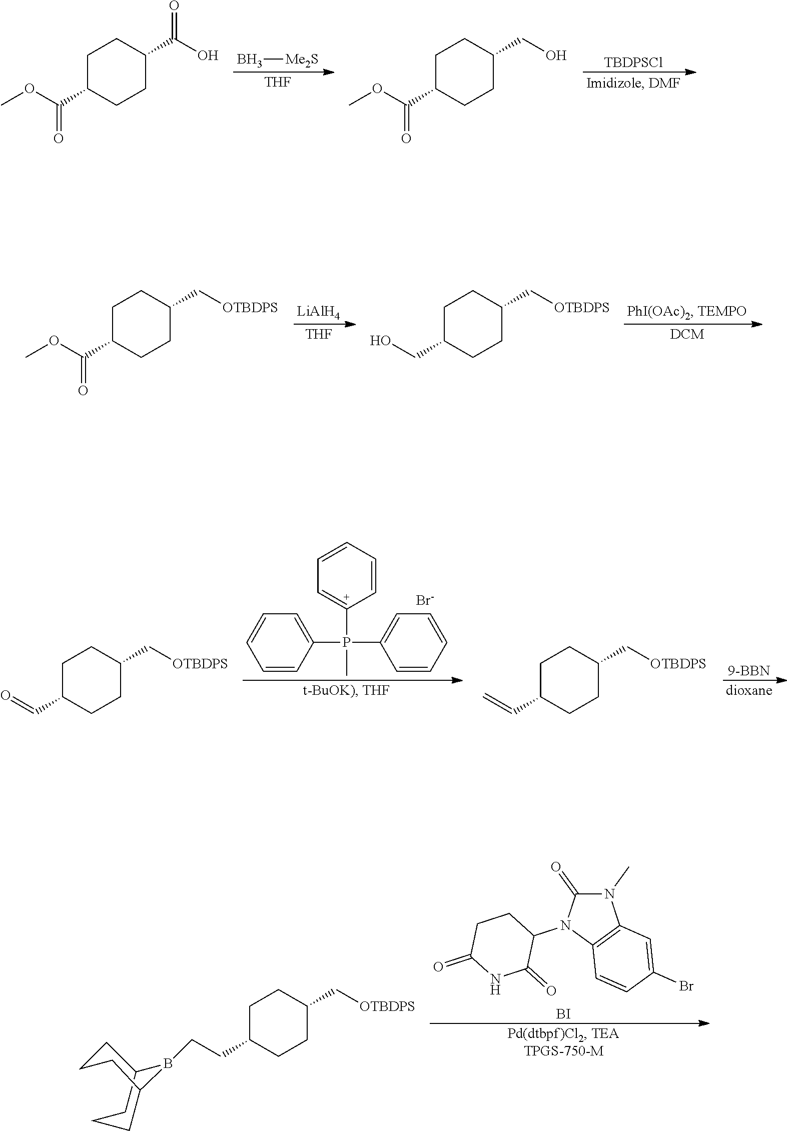

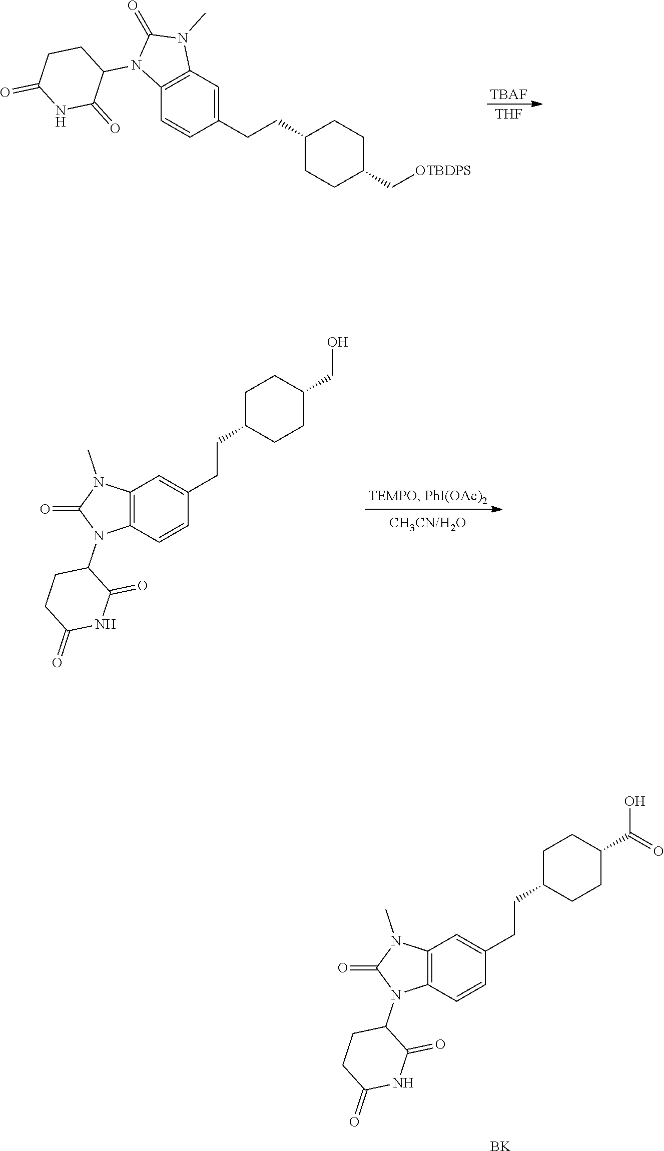

Step 1—Methyl (1S,4S)-4-(hydroxymethyl)cyclohexane-1-carboxylate. To a stirred solution of (1s,4s)-4-(methoxycarbonyl)cyclohexane-1-carboxylic acid (110.00 g, 590 mmol) in THF (1000 mL) was added a solution of 10 M BH3-Me2S (118.15 mL, 1181 mmol) dropwise at 0° C. under nitrogen atmosphere. The resulting solution was stirred for 2 h at rt under nitrogen atmosphere. On completion, the reaction was quenched with MeOH (400 mL) at 0° C. The resulting solution was concentrated under reduced pressure. The residue was purified by silica gel column chromatography, eluted with PE/EtOAc (5:1), to afford the title compound (94 g, 92% yield) as a colorless oil. ¹H NMR (400 MHz, Chloroform-d) δ 3.68 (s, 3H), 3.50 (d, J=6.4 Hz, 2H), 2.61-2.56 (m, 1H), 2.07-1.96 (m, 2H), 1.68-1.63 (m, 2H), 1.66-1.53 (m, 4H), 1.35-1.24 (m, 2H).

Step 2—Methyl (1S,4S)-4-[[(tert-butyldiphenylsilyl)oxy]methyl]cyclohexane-1-carboxylate. To a stirred solution of methyl (1s,4s)-4-(hydroxymethyl)cyclohexane-1-carboxylate (94.00 g, 545 mmol) and Imidazole (55.73 g, 81 mmol) in DMF (1500.00 mL) was added TBDPS-Cl (225.03 g, 819 mmol, 1.50 equiv) at 0° C. under nitrogen atmosphere. The resulting mixture was stirred for 16 h at rt under nitrogen atmosphere. On completion, the resulting mixture was diluted with water (1000 mL) and the resulting mixture was extracted with EtOAc (3×1000 mL). The combined organic layers were washed with brine (8×300 mL), and dried over anhydrous Na2SO4. After filtration, the filtrate was concentrated under reduced pressure. The residue was purified by silica gel column chromatography, eluted with PE/EtOAc (30:1) to afford the title compound (210 g, 94% yield) as a light yellow oil; ¹H NMR (400 MHz, Chloroform-d) δ 7.72-7.64 (m, 4H), 7.49-7.36 (m, 6H), 3.70 (s, 3H), 3.53 (d, J=6.4 Hz, 2H), 2.61-2.56 (m, 1H), 2.06-1.95 (m, 2H), 1.74-1.51 (m, 4H), 1.42-1.27 (m, 2H), 1.07 (s, 9H).

Step 3—[(1S,4S)-4-[[(tert-butyldiphenylsilyl)oxy]methyl]cyclohexyl]methanol. To a stirred solution of methyl (1s,4s)-4-[[(tert-butyldiphenylsilyl)oxy]methyl]cyclohexane-1-carboxylate (210 g, 511 mmol) in THF (2000 mL) was added LiAlH4 (409.13 mL, 1022 mmol) at 0° C. under nitrogen atmosphere. The resulting mixture was stirred at 0° C. to rt over 2 hrs under nitrogen atmosphere. On completion, the reaction was quenched with Water/Ice (600 mL) at 0° C. The resulting mixture was extracted with EtOAc (3×800 mL). The combined organic layers were washed with brine (2×300 mL), and dried over anhydrous Na2SO4. After filtration, the filtrate was concentrated under reduced pressure. The residue was purified by silica gel column chromatography, eluted with PE/EtOAc (10:1) to afford the title compound (190 g, 97% yield) as a light yellow oil. ¹H NMR (400 MHz, Chloroform-d) δ 7.74-7.66 (m, 4H), 7.50-7.36 (m, 6H), 3.58-3.54 (m, 4H), 1.82-1.67 (m, 2H), 1.62-1.44 (m, 5H), 1.43-1.27 (m, 3H), 1.08 (s, 9H).

Step 4—(1S,4S)-4-[[(tert-butyldiphenylsilyl)oxy]methyl]cyclohexane-1-carbaldehyde. To a stirred solution of [(1s,4s)-4-[[(tert-butyldiphenylsilyl)oxy]methyl]cyclohexyl]methanol (190.00 g, 497 mmol) in DCM (2500 mL) were added TEMPO (7.76 g, 49.7 mmol) and BAIB (319.89 g, 993 mmol) at rt under nitrogen atmosphere. The resulting mixture was stirred for 2 h at rt under nitrogen atmosphere. On completion, the reaction was quenched with Na203S2 (aq.) (300 mL) at 0° C. and the resulting mixture was extracted with DCM (3×1000 mL). The combined organic layers were washed with brine (2×500 mL), and dried over anhydrous Na2SO4. The resulting mixture was concentrated under reduced pressure. The residue was purified by silica gel column chromatography, eluted with PE/EtOAc (5:1), to afford the title compound (144 g, containing-2% of trans isomer, 76% yield) as a light yellow oil. ¹H NMR (400 MHz, Chloroform-d) δ 9.72 (s, 1H), 7.72-7.64 (m, 4H), 7.49-7.34 (m, 6H), 3.47 (d, J=6.3 Hz, 2H), 2.46-2.41 (m, 1H), 2.17-2.06 (m, 2H), 1.74-1.67 (m, 2H), 1.66-1.58 (m, 2H), 1.20-1.10 (m, 2H), 1.07 (s, 9H).

Step 5—Tert-butyl[(4-ethenylcyclohexyl)methoxy]diphenylsilane. To a stirred solution of methyltriphenylphosphanium bromide (202.74 g, 567 mmol) in THF (1400 mL) was added t-BuOK (472.93 mL, 472.93 mmol) dropwise at 0° C. under nitrogen atmosphere. The resulting mixture was stirred for 30 min at rt under nitrogen atmosphere. Next, to the mixture was added 4-[[(tert-butyldiphenylsilyl)oxy]methyl]cyclohexane-1-carbaldehyde (144 g, 378 mmol) dropwise at 0° C. under nitrogen atmosphere. The resulting mixture was stirred for 1 h at rt under nitrogen atmosphere. On completion, the reaction was quenched with water (500 mL) at rt and the aqueous layer was extracted with EtOAc (3×800 mL). The combined organic layers were washed with brine, dried over anhydrous Na2SO4. The residue was purified by silica gel column chromatography, eluted with PE to afford the title compound (124 g, contains of ˜2% of trans isomer, 87% yield) as a colorless oil. ¹H NMR (300 MHz, Chloroform-d) δ 7.75-7.66 (m, 4H), 7.51-7.35 (m, 6H), 5.94-5.83 m 1H), 5.08-4.93 (m, 2H), 3.58 (d, J=6.8 Hz, 2H), 2.28-2.24 (m, 1H), 1.78-1.74 (m, 1H), 1.62-1.42 (m, 8H), 1.09 (s, 9H).

Step 6—3-[5-[(E)-2-(4-[[(tert-butyldiphenylsilyl)oxy]methyl]cyclohexyl)ethenyl]-3-methyl-2-oxo-1,3-benzodiazol-1-yl]piperidine-2,6-dione. To a stirred mixture of 9-borabicyclo[3.3.1]nonane dimer (40.81 g, 334.4 mmol) in dioxane (180.00 mL) was added tert-butyl[(4-ethenylcyclohexyl)methoxy]diphenylsilane (116.89 g, 308.73 mmol) dropwise at rt under argon atmosphere. The resulting mixture was stirred for 1 h at 50° C. under argon atmosphere. To a stirred mixture of 3-(5-bromo-3-methyl-2-oxo-1,3-benzodiazol-1-yl)piperidine-2,6-dione (87.00 g, 257.3 mmol, Intermediate BI), Pd(DtBPF)Cl2 (8.38 g, 12.9 mmol) in TPGS-750-M (290 mL) were added TEA (78.10 g, 771.8 mmol) and the 9-BBN solution above in turns at 50° C. under argon atmosphere. The resulting mixture was stirred for additional 3 h at 50° C. On completion, the reaction was quenched with water (500 mL) at rt. The resulting mixture was extracted with EA (3×1000 mL). The combined organic layers were washed with brine, and dried over anhydrous Na2SO4. The residue was purified by silica gel column chromatography, eluted with DCM/EA (10:1 to 1:1), to afford the title compound (89.6 g, contains of ˜2% of trans isomer, 55% yield) as a light brown solid. ¹H NMR (400 MHz, Chloroform-d) δ 8.49 (s, 1H), 7.71-7.68 (m, 4H), 7.47-7.39 (m, 6H), 6.93-6.83 (m, 2H), 6.76-6.72 (m, 1H), 5.25 (dd, J=12.6, 5.3 Hz, 1H), 3.55-3.51 (m, 2H), 3.45 (s, 3H), 2.97-2.84 (m, 2H), 2.81-2.61 (m, 3H), 2.28-2.20 (m, 1H), 1.88-1.84 (m, 1H), 1.77-1.73 (m, 1H), 1.58-1.47 (m, 8H), 1.39-1.32 (m, 2H), 1.08 (s, 9H). LC/MS (ESI, m/z): [(M+H)]+=638.5.

Step 7—3-(3-methyl-2-oxo-5-[2-[(1s,4s)-4-(hydroxymethyl)cyclohexyl]ethyl]-1,3-benzodiazol-1-yl)piperidine-2,6-dione. To a stirred solution of 3-(3-methyl-2-oxo-5-[2-[(1s,4s)-4-[[(tert-butyldiphenylsilyl)oxy]methyl] cyclohexyl]ethyl]-1,3-benzodiazol-1-yl)piperidine-2,6-dione (90.00 g, 141.0 mmol) in THF (900 mL) was added TBAF (282.18 mL, 282.18 mmol) dropwise at 0° C. under air atmosphere. The resulting mixture was stirred for 1 h at rt under air atmosphere. The resulting mixture was concentrated under reduced pressure. The residue was purified by silica gel column chromatography, eluted with DCM/EA (10:1 to 1:2), to afford the title compound (45 g, contains of ˜2% of trans isomer, 80% yield) as a white solid. 1H NMR (400 MHz, Chloroform-d) δ 8.98 (s, 1H), 6.90-6.82 (m, 2H), 6.75-6.71 (m, 1H), 5.24 (dd, J=12.7, 5.3 Hz, 1H), 3.54 (d, J=6.9 Hz, 2H), 3.42 (s, 3H), 2.91-2.80 (m, 2H), 2.77-2.59 (m, 3H), 2.23-2.12 (m, 1H), 1.69-1.65 (m, 1H), 1.63-1.48 (m, 8H), 1.47-1.37 (m, 4H). LC/MS (ESI, m/z): [(M+H)]+=400.2.

Step 8—(1s,4s)-4-[2-[1-(2,6-dioxopiperidin-3-yl)-3-methyl-2-oxo-1,3-benzodiazol-5-yl]ethyl]cyclohexane-1-carboxylic acid. To a stirred solution of 3-(5-[2-[4-(hydroxymethyl)cyclohexyl]ethyl]-3-methyl-2-oxo-1,3-benzodiazol-1-yl)piperidine-2,6-dione (46.00 g, 115.15 mmol) in ACN (500 mL) and H₂O (50 mL) were added BAIB (185.44 g, 575.73 mmol) and TEMPO (3.60 g, 23.03 mmol) at rt under nitrogen atmosphere. The resulting mixture was stirred for 2 h at rt under nitrogen atmosphere. On completion, the resulting mixture was concentrated under reduced pressure. The residue was purified by trituration with Et2O (3×300 mL). The crude product was re-crystallized from DMSO/water (1:1, 300 mL) at 80° C. and washed with DMSO/water (1:5, 3×100 mL) to afford the title compound (35 g, 74% yield) as a pink solid. 1H NMR (300 MHz, DMSO-d6) δ 12.05 (s, 1H), 11.10 (s, 1H), 7.06-6.96 (m, 2H), 6.88-6.84 (m, 1H), 5.34 (dd, J=12.7, 5.4 Hz, 1H), 3.32 (s, 3H), 2.97-2.84 (m, 1H), 2.75-2.68 (m, 1H), 2.65-2.58 (m, 3H), 2.47-2.44-2.40 (m, 1H), 2.03-1.97 (m, 1H), 1.91-1.86 (m, 2H), 1.62-1.57 (m, 2H), 1.51-1.45 (m, 4H), 1.40-1.32 (m, 1H), 1.25-1.17 (m, 2H). LC/MS (ESI, m/z): [(M+H)]⁺=414.2.

### Tert-butyl N-[(5S,8S,10aR)-8-[[(1S)-3-carbamoyl-1-[[(4-isopropylphenyl)methyl]carbamoyl]propyl]carbamoyl]-6-oxo-octahydro-1H-pyrrolo[1,2-a][1,5]diazocin-5-yl]carbamate (Intermediate BL)

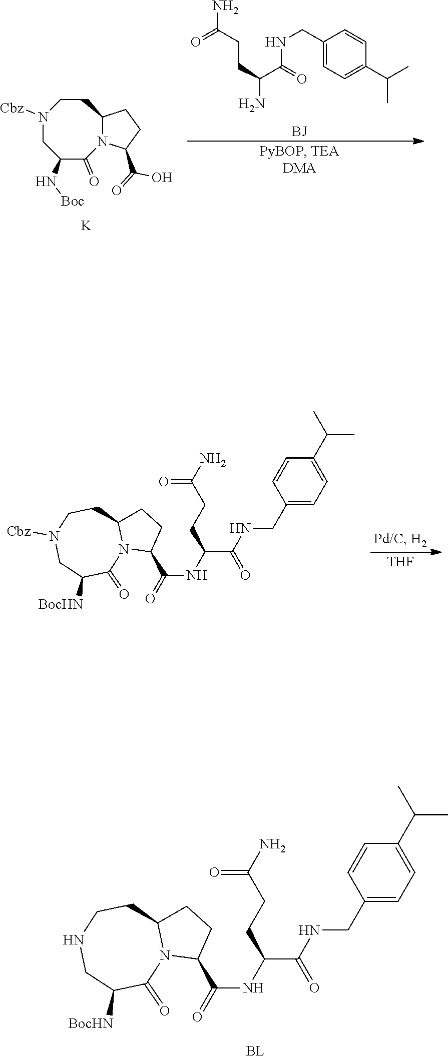

Step 1—Benzyl (5S,8S,10aR)-5-[(tert-butoxycarbonyl)amino]-8-[[(1S)-3-carbamoyl-1-[[(4-isopropylphenyl)methyl]carbamoyl]propyl]carbamoyl]-6-oxo-octahydropyrrolo[1,2-a][1,5]diazocine-3-carboxylate. To a stirred mixture of (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (3.00 g, 6.50 mmol, Intermediate K) and (2S)-2-amino-N-[(4-isopropylphenyl)methyl]pentanediamide (1.98 g, 7.15 mmol, Intermediate BJ) in DMA (25 mL) were added TEA (4.52 mL, 32.50 mmol) and PyBOP (4.06 g, 7.80 mmol) at 0° C. under nitrogen atmosphere. Then the resulting mixture was stirred for 1 h at rt under nitrogen atmosphere. On completion, the residue was purified by reverse phase flash chromatography (Column: Spherical C18, 20-40 um, 330 g; Mobile Phase A: Water (10 mmol/L FA); Mobile Phase B: ACN; Flow rate: 80 mL/min; Gradient: 5%-5% B, 10 min, 35% B-65% B gradient in 30 min; Detector: 220 nm; the fractions containing the desired product were collected at 57% B) and concentrated under reduced pressure to afford the title compound (4.8 g, 79% yield) as a white solid. 1H NMR (400 MHz, DMSO-d6) δ 8.32-8.27 (m, 1H), 8.20-8.15 (m, 1H), 7.44-7.26 (m, 5H), 7.20-7.13 (m, 5H), 6.93 (d, J=7.7 Hz, 1H), 6.85-6.67 (m, 1H), 5.17-5.01 (m, 2H), 4.47-4.30 (m, 2H), 4.29-4.09 (m, 4H), 3.81-3.72 (m, 1H), 3.60-3.52 (m, 1H), 3.22-306 (m, 1H), 2.89-2.77 (m, 1H), 2.21-2.10 (m, 3H), 2.03-1.85 (m, 2H), 1.85-1.58 (m, 6H), 1.38 (s, 9H), 1.19-1.13 (m, 6H). LC/MS (ESI, m/z): [(M+H)]⁺=721.4.

Step 2—Tert-butyl N-[(5S,8S,10aR)-8-[[(1S)-3-carbamoyl-1-[[(4-isopropylphenyl)methyl]carbamoyl]propyl]carbamoyl]-6-oxo-octahydro-1H-pyrrolo[1,2-a][1,5]diazocin-5-yl]carbamate. To a solution of benzyl (5S,8S,10aR)-5-[(tert-butoxycarbonyl)amino]-8-[[(1S)-3-carbamoyl-1-[[(4-isopropylphenyl)methyl]carbamoyl]propyl]carbamoyl]-6-oxo-octahydropyrrolo[1,2-a][1,5]diazocine-3-carboxylate (8.40 g, 11.65 mmol) in THF (120 mL) was added Pd/C (10 wt %, 1.24 g, 1.17 mmol) under nitrogen atmosphere. The reaction system was degassed under vacuum and purged with H₂ several times. Then the mixture was hydrogenated under H2 balloon (A1 atm) at 25° C. for 2 h. After completion of the reaction, Pd/C was filtered through celite and the filter cake was washed with THF (3×10 mL). The corresponding filtrate was concentrated under reduced pressure to provide the title compound (5.6 g, 82% yield) as a white solid. ¹H NMR (300 MHz, DMSO-d6) δ 8.94 (d, J=8.2 Hz, 1H), 7.95-7.88 (m, 1H), 7.28 (s, 1H), 7.20-7.13 (m, 4H), 6.81-6.67 (m, 2H), 4.76-4.60 (m, 1H), 4.42-4.34 (m, 2H), 4.30-4.09 (m, 3H), 3.22-3.15 (m, 1H), 2.92-2.68 (m, 2H), 2.66-2.57 (m, 1H), 2.36-2.28 (m, 1H), 2.09-1.96 (m, 4H), 1.95-1.81 (m, 2H), 1.81-1.63 (m, 4H), 1.55-1.46 (m, 1H), 1.39 (s, 9H), 1.23-1.15 (m, 6H); LC/MS (ESI, m/z): [(M+H)]+=587.4.

### (2S)-1-[(2S)-2-[(tert-butoxycarbonyl)aminol-4-methylpentanoyl]pyrrolidine-2-carboxylic acid (Intermediate BM) (CAS #64205-66-9)

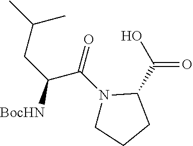 [SMILES: CC(C)C[C@H](NC(=O)OC(C)(C)C)C(=O)N1CCC[C@H]1C(=O)O]

### (2S,4R)-1-[(2S)-2-(5-[3-[(2S)-2-amino-4-carbamoylbutoxy]-2-fluorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate BN)

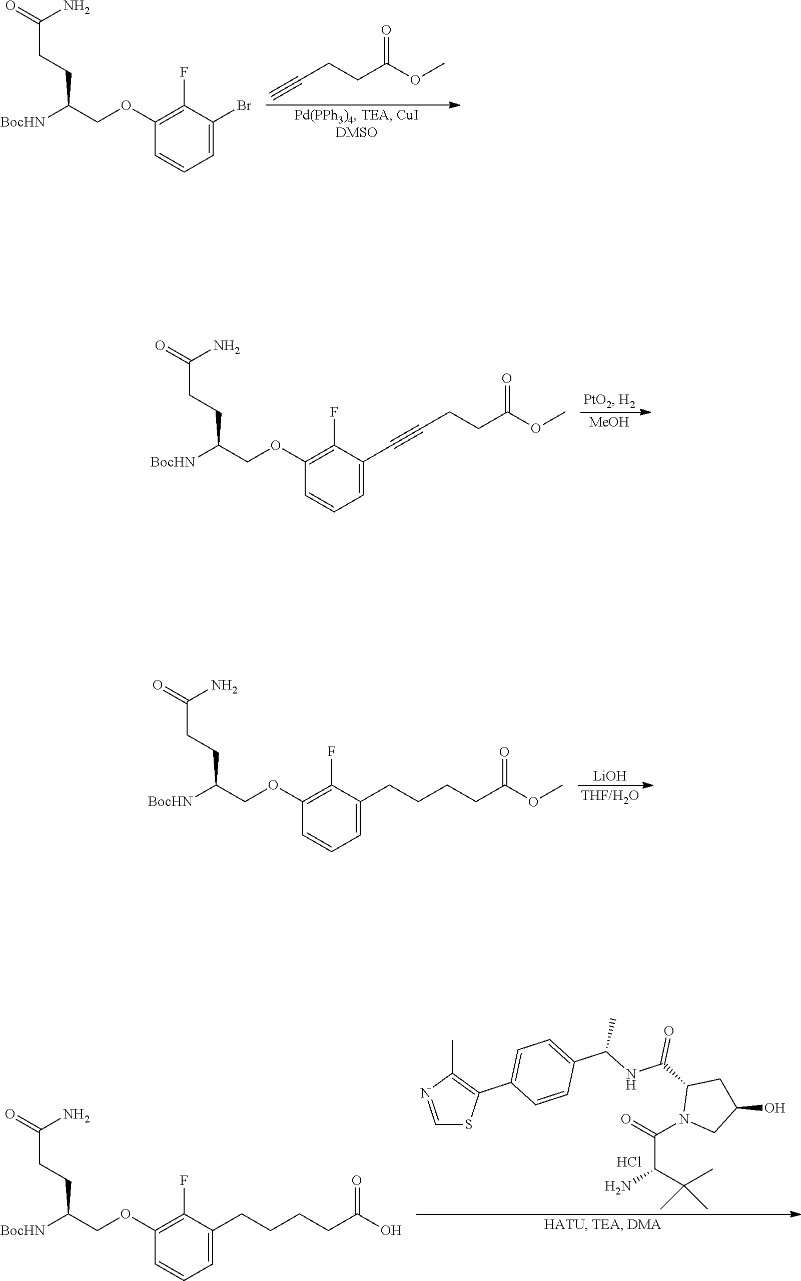

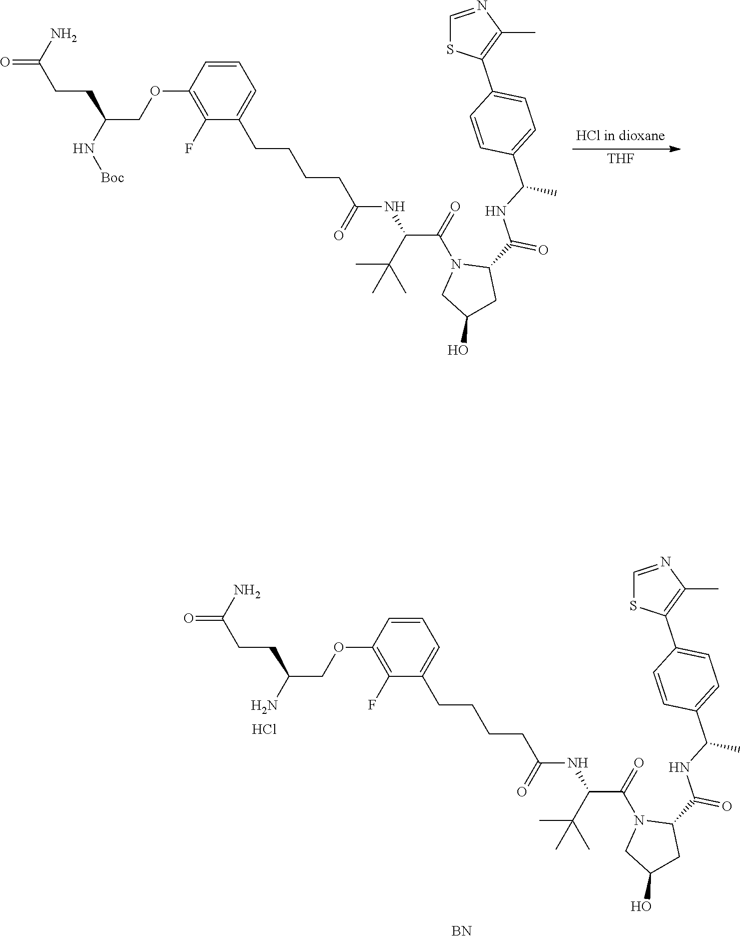

Step 1—Methyl 5-[3-[(2S)-2-[(tert-butoxycarbonyl)amino]-4-carbamoylbutoxy]-2-fluorophenyl]pent-4-ynoate. To a stirred solution of tert-butyl N-[(2S)-1-(3-bromo-2-fluorophenoxy)-4-carbamoylbutan-2-yl]carbamate (600.00 mg, 1.48 mmol, synthesized via Step 1 of Intermediate Q) and methyl pent-4-ynoate (498.02 mg, 4.44 mmol) in DMSO (5 mL) were added TEA (2.68 mL, 26.44 mmol) dropwise, CuI (28.20 mg, 0.148 mmol) and Pd(PPh3)4 (171.08 mg, 0.148 mmol) in portions at rt under nitrogen atmosphere. The resulting mixture was stirred for 2 h at 80° C. under nitrogen atmosphere. On completion, the reaction mixture was concentrated under reduced pressure. The residue was purified by reverse flash chromatography (Column, C18 silica gel; mobile phase, MeCN in water (10 mmol/L NH4HCO3), 40% to 60% gradient in 25 min; detector, UV 220 nm) to afford the title compound (543 mg, 84% yield) as a light yellow solid. ¹H NMR (400 MHz, DMSO-d6) δ 7.26 (s, 1H), 7.18 (t, J=8.0 Hz, 1H), 7.07 (t, J=8.0 Hz, 1H), 6.99-6.95 (m, 1H), 6.84 (d, J=8.4 Hz, 1H), 6.73 (s, 1H), 4.03-3.90 (m, 2H), 3.75-3.71 (m, 1H), 3.64 (s, 3H), 2.72 (t, J=6.7 Hz, 2H), 2.65-2.61 (m, 2H), 2.12 (q, J=7.1 Hz, 2H), 1.87-1.73 (m, 1H), 1.64-1.52 (m, 1H), 1.39 (s, 9H); LC/MS (ESI, m/z): [(M+1)]+=437.3.

Step 2—methyl 5-[3-[(2S)-2-[(tert-butoxycarbonyl)amino]-4-carbamoylbutoxy]-2-fluorophenyl]pentanoate. To a stirred solution of methyl 5-[3-[(2S)-2-[(tert-butoxycarbonyl)amino]-4-carbamoylbutoxy]-2-fluorophenyl]pent-4-ynoate (543.00 mg, 1.244 mmol) in MeOH (10.00 mL) was added PtO2 (28.25 mg, 0.124 mmol) in portions at rt under nitrogen atmosphere. The resulting mixture was stirred for 16 h at rt under hydrogen atmosphere. On completion, the reaction mixture was filtered and the filter cake was washed with MeOH (3×10 mL). The filtrate was concentrated under reduced pressure to afford the title compound (511 mg, 93% yield) as a light yellow solid. ¹H NMR (400 MHz, DMSO-d6) δ 7.25 (d, J=16.8 Hz, 1H), 7.06-6.93 (m, 2H), 6.84-6.80 (m, 2H), 6.74 (s, 1H), 3.91 (d, J=6.0 Hz, 2H), 3.74 (t, J=5.1 Hz, 1H), 3.58 (s, 3H), 3.25-3.21 (m, 2H), 2.58 (d, J=7.2 Hz, 2H), 2.37-2.25 (m, 2H), 2.12-2.08 (m, 2H), 1.84-1.80 (m, 1H), 1.64-1.55 (m, 1H), 1.56-1.52 (m, 2H), 1.39 (s, 9H); LC/MS (ESI, m/z): [(M+1)]⁺=441.4.

Step 3—5-[3-[(2S)-2-[(tert-butoxycarbonyl)amino]-4-carbamoylbutoxy]-2-fluorophenyl]pentanoic acid. To a stirred solution of methyl 5-[3-[(2S)-2-[(tert-butoxycarbonyl)amino]-4-carbamoylbutoxy]-2-fluorophenyl]pentanoate (511.00 mg, 1.16 mmol) in THF (15.00 mL) was added a solution of LiOH (277.80 mg, 11.60 mmol) in H₂O (15.00 mL) dropwise at rt. The resulting mixture was stirred for 3 h at rt. On completion, the reaction mixture was acidified to pH 4 with conc. HCl. The resulting mixture was then extracted with CH₂Cl₂ (3×20 mL). The combined organic layers were washed with brine (2×20 mL), and dried over anhydrous Na₂SO₄. After filtration, the filtrate was concentrated under reduced pressure to afford the title compound (429 mg, 87% yield) as a white solid. ¹H NMR (400 MHz, DMSO-d6) δ 11.96 (s, 1H), 7.27 (s, 1H), 7.06-6.94 (m, 2H), 6.84-6.80 (m, 2H), 6.74 (s, 1H), 3.91 (d, J=6.1 Hz, 2H), 3.74 (s, 1H), 2.59 (t, J=7.0 Hz, 2H), 2.23 (t, J=6.9 Hz, 2H), 2.12-2.08 (m, 2H), 1.91-1.73 (m, 1H), 1.61-1.50 (m, 5H), 1.39 (s, 9H); LC/MS (ESI, m/z): [(M+1)]+=427.3.

Step 4—tert-butyl N-[(2S)-4-carbamoyl-1-[2-fluoro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate. To a stirred mixture of 5-[3-[(2S)-2-[(tert-butoxycarbonyl)amino]-4-carbamoylbutoxy]-2-fluorophenyl]pentanoic acid (429.00 mg, 1.01 mmol) and HATU (458.97 mg, 1.207 mmol) in DMA (5 mL) were added TEA (0.42 mL, 4.2 mmol) dropwise and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (580.66 mg, 1.207 mmol) in turns at rt under nitrogen atmosphere. The resulting mixture was stirred for 16 h at rt under nitrogen atmosphere. On completion, the reaction mixture was directly purified by reverse flash chromatography (Column, C18 silica gel; mobile phase, MeCN in water (10 mmol/L NH₄HCO₃), 40% to 60% gradient in 25 min; detector, UV 254 nm) to afford the title compound (763 mg, 89% yield) as a yellow solid. ¹H NMR (400 MHz, DMSO-d6) δ 8.99 (d, J=2.2 Hz, 1H), 8.38 (d, J=7.8 Hz, 1H), 7.82 (d, J=9.2 Hz, 1H), 7.44 (dd, J=8.2, 2.0 Hz, 2H), 7.39 (dd, J=8.3, 1.9 Hz, 2H), 7.27 (s, 1H), 7.06-6.94 (m, 2H), 6.83 (td, J=9.3, 8.7, 5.7 Hz, 2H), 6.74 (s, 1H), 5.10 (s, 1H), 4.93 (p, J=7.3 Hz, 1H), 4.52 (d, J=9.2 Hz, 1H), 4.46-4.42 (m, 1H), 4.29 (s, 1H), 3.91 (d, J=6.0 Hz, 2H), 3.74 (s, 1H), 3.61 (s, 1H), 2.58 (d, J=7.5 Hz, 2H), 2.46 (s, 3H), 2.31-2.27 (m, 1H), 2.15-2.11 (m, 3H), 2.03-1.99 (m, 1H), 1.83-1.79 (m, 2H), 1.68-1.56 (m, 1H), 1.57-1.45 (m, 4H), 1.39 (s, 9H), 1.35-1.31 (m, 1H), 1.25 (s, 3H), 0.94 (s, 9H); LC/MS (ESI, m/z): [(M+1)]+=853.7.

Step 5—(2S,4R)-1-[(2S)-2-(5-[3-[(2S)-2-amino-4-carbamoylbutoxy]-2-fluorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-fluoro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate (300.00 mg, 0.352 mmol) in THF (5 mL) was added 4 M HCl (gas) in 1,4-dioxane (5 mL) dropwise at rt under nitrogen atmosphere. The resulting mixture was stirred for 2 h at rt under nitrogen atmosphere. On completion, the reaction mixture was concentrated under reduced pressure to afford the title compound (271 mg) as a white solid. ¹H NMR (400 MHz, DMSO-d6) δ 9.03 (s, 1H), 8.40 (d, J=7.8 Hz, 1H), 8.30 (s, 2H), 7.83 (d, J=9.3 Hz, 1H), 7.45 (d, J=8.2 Hz, 2H), 7.39 (d, J=8.4 Hz, 2H), 7.06 (d, J=6.2 Hz, 2H), 6.98-6.86 (m, 1H), 4.92 (p, J=7.5 Hz, 1H), 4.51 (d, J=9.3 Hz, 1H), 4.44-4.40 (m, 1H), 4.24-4.20 (m, 1H), 4.17-4.08 (m, 1H), 3.69-3.58 (m, 5H), 2.61 (s, 2H), 2.47 (s, 3H), 2.37-2.25 (m, 2H), 2.21-2.11 (m, 1H), 2.08-1.97 (m, 1H), 1.93-1.89 (m, 1H), 1.85-1.72 (m, 2H), 1.63-1.58 (m, 1H), 1.55-1.51 (m, 4H), 1.49-1.43 (m, 2H), 1.38 (d, J=7.0 Hz, 3H), 0.93 (s, 9H); LC/MS (ESI, m/z): [(M+H)]+=753.6.

### (S)-2-amino-N1-(4-(methylsulfonyl)benzyl)pentanediamide (Intermediate BO)

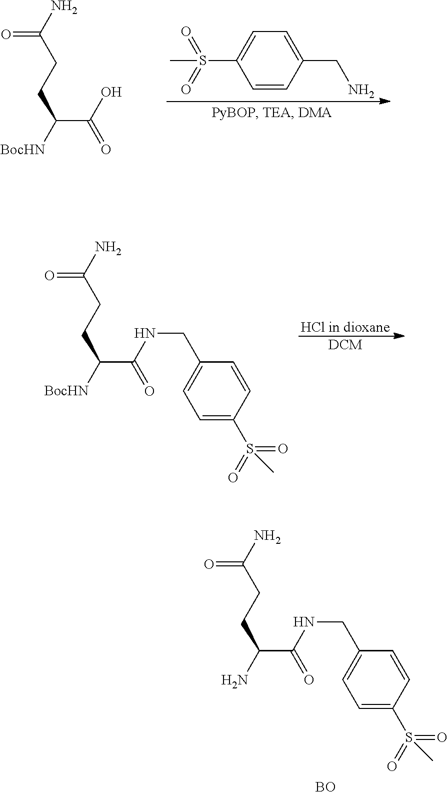 [SMILES: CC(C)(C)OC(=O)N[C@@H](CCC(N)=O)C(=O)O ; CS(=O)(=O)c1ccc(CN)cc1 ; CC(C)(C)OC(=O)N[C@@H](CCC(N)=O)C(=O)NCc1ccc(S(C)(=O)=O)cc1 ; CS(=O)(=O)c1ccc(CNC(=O)[C@@H](N)CCC(N)=O)cc1 ; "BO"]

Step 1—Tert-butyl (S)-(5-amino-1-((4-(methylsulfonyl)benzyl)amino)-1,5-dioxopentan-2-yl)carbamate. To a stirred mixture of (2S)-2-[(tert-butoxycarbonyl)amino]-4-carbamoylbutanoic acid (1.00 g, 4.06 mmol, CAS #13726-85-7) and 1-(4-methanesulfonylphenyl)methanamine (902.64 mg, 4.87 mmol) in DMA (15 mL) were added TEA (1.23 g 12.18 mmol) and PyBOP (3.17 g, 6.09 mmol) at rt under nitrogen atmosphere. The resulting mixture was stirred for 2 h at rt under nitrogen atmosphere. On completion, the residue was purified by reverse phase flash with the following conditions (Column: Spherical C18, 20-40 um, 120 g; Mobile Phase A: Water (0.05% FA), Mobile Phase B: ACN; Flow rate: 45 mL/min; Gradient (B %): 5%˜5%, 4 min; 15%˜45%, 30 min; 95%, 5 min; Detector: 254 nm; Rt: 20 min.) to afford the title compound (1.6 g, 95% yield) as a white solid. ¹H NMR (400 MHz, DMSO-d6) δ 8.48 (t, J=6.0 Hz, 1H), 7.85 (d, J=8.0 Hz, 2H), 7.51 (d, J=8.1 Hz, 2H), 7.28 (s, 1H), 7.02 (d, J=7.7 Hz, 1H), 6.78 (s, 1H), 4.38 (d, J=5.9 Hz, 2H), 3.96-3.86 (m, 1H), 3.19 (s, 3H), 2.16-2.04 (m, 2H), 1.92-1.82 (m, 1H), 1.79-1.67 (m, 1H), 1.40 (s, 9H); LC/MS (ESI, m/z): [(M+1)]+=414.1.

Step 2—(S)-2-amino-N1-(4-(methylsulfonyl)benzyl)pentanediamide hydrochloride. To a stirred solution of tert-butyl N-[(1S)-3-carbamoyl-1-[[(4-methanesulfonylphenyl)methyl]carbamoyl]propyl]carbamate (1.60 g, 3.87 mmol) in DCM (20 mL) was added HCl (gas) in 1,4-dioxane (5.00 mL, 20 mmol) dropwise at rt under nitrogen atmosphere. The resulting mixture was stirred for 1 h at rt under nitrogen atmosphere. On completion, the resulting mixture was concentrated under vacuum to afford the title compound (1.4 g, quantitative crude yield) as a white solid. ¹H NMR (400 MHz, DMSO-d6) δ 9.35 (t, J=6.0 Hz, 1H), 8.43 (m, 2H), 7.89 (d, J=8.4 Hz, 2H), 7.58 (d, J=8.4 Hz, 2H), 7.53 (s, 1H), 6.95 (s, 1H), 4.52-4.37 (m, 2H), 3.93-3.87 (m, 1H), 3.21 (s, 3H), 2.29-2.16 (m, 2H), 2.07-1.92 (m, 2H); LC/MS (ESI, m/z): [(M+1)]+=314.1.

### Tert-butyl ((3S,6S)-6-(((S)-5-amino-1-((4-(methylsulfonyl)benzyl)amino)-1,5-dioxopentan-2-yl)carbamoyl)-9-bromo-4-oxo-1,2,3,4,6,7-hexahydroazepino[3,2,1-hi]indol-3-yl)carbamate (Intermediate BP)

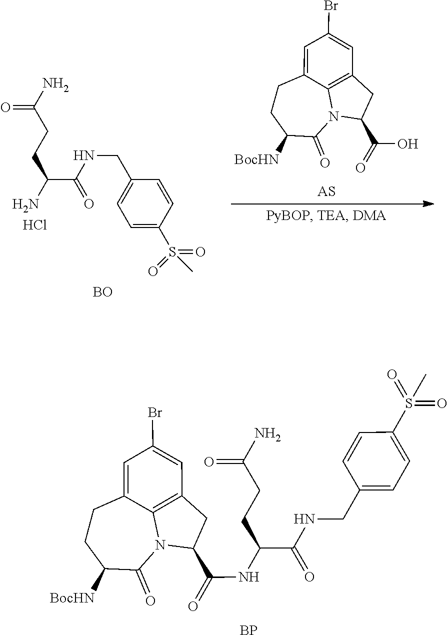

To a stirred mixture of (2S)-2-amino-N-[(4-methanesulfonylphenyl)methyl]pentanediamide (1.00 g, 3.19 mmol, Intermediate BO) and (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (1.63 g, 3.83 mmol, Intermediate AS) in DMA (15 mL) were added TEA (0.97 g, 9.57 mmol) and PyBOP (1.99 g, 3.83 mmol) at rt under nitrogen atmosphere. The resulting mixture was stirred for 2 h at rt under nitrogen atmosphere. On completion, the residue product was purified by reverse phase flash (Column: Spherical C18, 20-40 um, 330 g; Mobile Phase A: Water (0.05% FA), Mobile Phase B: ACN; Flow rate: 80 mL/min; Gradient (B %): 5%˜5%, 6 min; 25%˜55%, 30 min; 95%, 5 min; Detector: 254 nm; Rt: 38 min.) to afford the title compound (1.6 g, 70% yield) as a white solid. ¹H NMR (400 MHz, DMSO-d6) δ 8.56 (t, J=6.1 Hz, 1H), 8.30 (d, J=7.7 Hz, 1H), 7.85 (d, J=8.1 Hz, 2H), 7.48 (d, J=8.0 Hz, 2H), 7.30 (s, 1H), 7.27 (d, J=7.8 Hz, 2H), 7.13 (d, J=8.1 Hz, 1H), 6.79 (s, 1H), 5.16-5.08 (m, 1H), 4.45-4.29 (m, 2H), 4.21-4.17 (m, 1H), 4.05 (t, J=9.2 Hz, 1H), 3.49-3.38 (m, 1H), 3.19 (s, 3H), 3.11-3.02 (m, 2H), 3.01-2.93 (m, 2H), 2.15-2.06 (m, 2H), 2.05-2.00 (m, 1H), 2.00-1.72 (m, 2H), 1.39 (s, 9H); LC/MS (ESI, m/z): [(M+1)]+=720.0, 722.2.

### (S)-4-(3-((5-amino-2-((tert-butoxycarbonyl)amino)-5-oxopentyl)oxy)-2-chloro-5-methylphenyl)butanoic acid (Intermediate BQ)

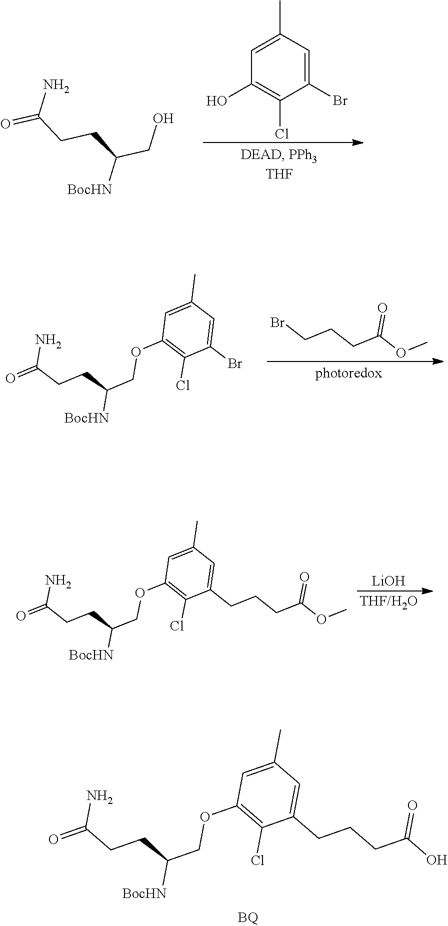

Step 1—Tert-butyl (S)-(5-amino-1-(3-bromo-2-chloro-5-methylphenoxy)-5-oxopentan-2-yl)carbamate. To a stirred mixture of 3-bromo-2-chloro-5-methylphenol (2.83 g, 12.79 mmol) and PPh₃ (3.66 g, 13.95 mmol) in THF (20 mL) was added DEAD (2.43 g, 13.95 mmol) dropwise at 0° C. under nitrogen atmosphere. The resulting mixture was stirred for 20 min at 0° C. under nitrogen atmosphere. To the above mixture was added tert-butyl N-[(2S)-4-carbamoyl-1-hydroxybutan-2-yl]carbamate (2.70 g, 11.62 mmol, CAS #133565-42-1) at rt. The resulting mixture was stirred for additional 4 h at rt. On completion, the resulting mixture was concentrated under reduced pressure. The residue was purified by silica gel column chromatography, eluted with PE/EtOAc (50%˜100%), to afford the title compound (1.4 g, 28% yield) as a white solid. 1H NMR (400 MHz, Chloroform-d) δ 7.09 (d, J=1.8 Hz, 1H), 6.67 (d, J=1.9 Hz, 1H), 6.36 (s, 1H), 5.70 (s, 1H), 5.17 (d, J=8.2 Hz, 1H), 4.08-4.01 (m, 3H), 2.37 (t, J=6.9 Hz, 2H), 2.30 (s, 3H), 2.08-2.02 (m, 2H), 1.46 (s, 9H); LC/MS (ESI, m/z): [(M+H)]+=435.0, 437.0.

Step 2—Methyl (S)-4-(3-((5-amino-2-((tert-butoxycarbonyl)amino)-5-oxopentyl)oxy)-2-chloro-5-methylphenyl)butanoate. To a stirred mixture of tert-butyl N-[(2S)-1-(3-bromo-2-chloro-5-methylphenoxy)-4-carbamoylbutan-2-yl]carbamate (600.00 mg, 1.38 mmol) and methyl 4-bromobutanoate (299.13 mg, 1.65 mmol) in DME (10 mL) were added tris(trimethylsilyl)silane (342.40 mg, 1.38 mmol), IR[DF(CF3)PPY]2(DTBPY)PF6 (15.45 mg, 0.014 mmol) and Na2CO3 (437.83 mg, 4.13 mmol) at rt under nitrogen atmosphere. To the above mixture was added 4-tert-butyl-2-(4-tert-butylpyridin-2-yl)pyridine (3.70 mg, 0.014 mmol) and 1,2-dimethoxyethane dihydrochloride nickel (3.03 mg, 0.014 mmol) at rt under nitrogen atmosphere. The resulting mixture was stirred for an additional 5 h with photoredox radiation at 450 nm. On completion, the resulting mixture was concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 35%-60% B in 25 min; Flow rate: 80 mL/min; Detector: 220/254 nm; desired fractions were collected at 55% B) and concentrated under reduced pressure to afford the title compound (260 mg, 41% yield) as a white solid. 1H NMR (300 MHz, Chloroform-d) δ 6.70-6.68 (m, 1H), 6.60-6.58 (m, 1H), 6.39 (s, 1H), 5.41 (s, 1H), 5.16 (d, J=8.5 Hz, 1H), 4.06-4.03 (m, 3H), 3.69 (s, 3H), 2.74 (t, J=8.7 Hz, 2H), 2.37 (t, J=7.3 Hz, 4H), 2.30 (s, 3H), 2.14-2.02 (m, 2H), 2.00-1.91 (m, 2H), 1.47 (s, 9H); LC/MS (ESI, m/z): [(M+H)]+=457.2.

Step 3—(S)-4-(3-((5-amino-2-((tert-butoxycarbonyl)amino)-5-oxopentyl)oxy)-2-chloro-5-methylphenyl)butanoic acid. To a stirred mixture of methyl 4-[3-[(2S)-2-[(tert-butoxycarbonyl)amino]-4-carbamoylbutoxy]-2-chloro-5-methylphenyl]butanoate (260.00 mg, 0.569 mmol) in THF (3.00 mL) were added LiOH·H₂O (238.77 mg, 5.69 mmol) and H₂O (3.00 mL) at rt. The resulting mixture was stirred for 2 h at room temperature under air atmosphere. On completion, the residue was purified by reverse phase flash chromatography (Column: WelFlash™ C18-I, 20-40 μm, 120 g; Eluent A: Water (plus 10 mmol/L FA); Eluent B: ACN; Gradient: 30%-50% B in 25 min; Flow rate: 60 mL/min; Detector: 220/254 nm; desired fractions were collected at 45% B) and concentrated under reduced pressure to afford the title compound (240 mg, 95% yield) as a white solid. 1H NMR (300 MHz, DMSO-d6) δ 11.99 (s, 1H), 7.28 (s, 1H), 6.90-6.65 (m, 3H), 3.91 (d, J=6.1 Hz, 2H), 3.85-3.65 (m, 1H), 2.66 (dd, J=8.7, 6.6 Hz, 2H), 2.28 (s, 3H), 2.24 (d, J=7.3 Hz, 2H), 2.18-2.10 (m, 2H), 1.92-1.70 (m, 4H), 1.69-1.56 (m, 1H), 1.40 (s, 9H); LC/MS (ESI, m/z): [(M+H)]+=443.1.

### (2S,4R)-1-((S)-2-(4-(2-chloro-3-(((S)-2,5-diamino-5-oxopentyl)oxy)-5-methylphenyl)butanamido)-3,3-dimethylbutanoyl)-4-hydroxy-N—((S)-1-(4-(4-methylthiazol-5-yl)phenyl)ethyl)pyrrolidine-2-carboxamide (Intermediate BR)

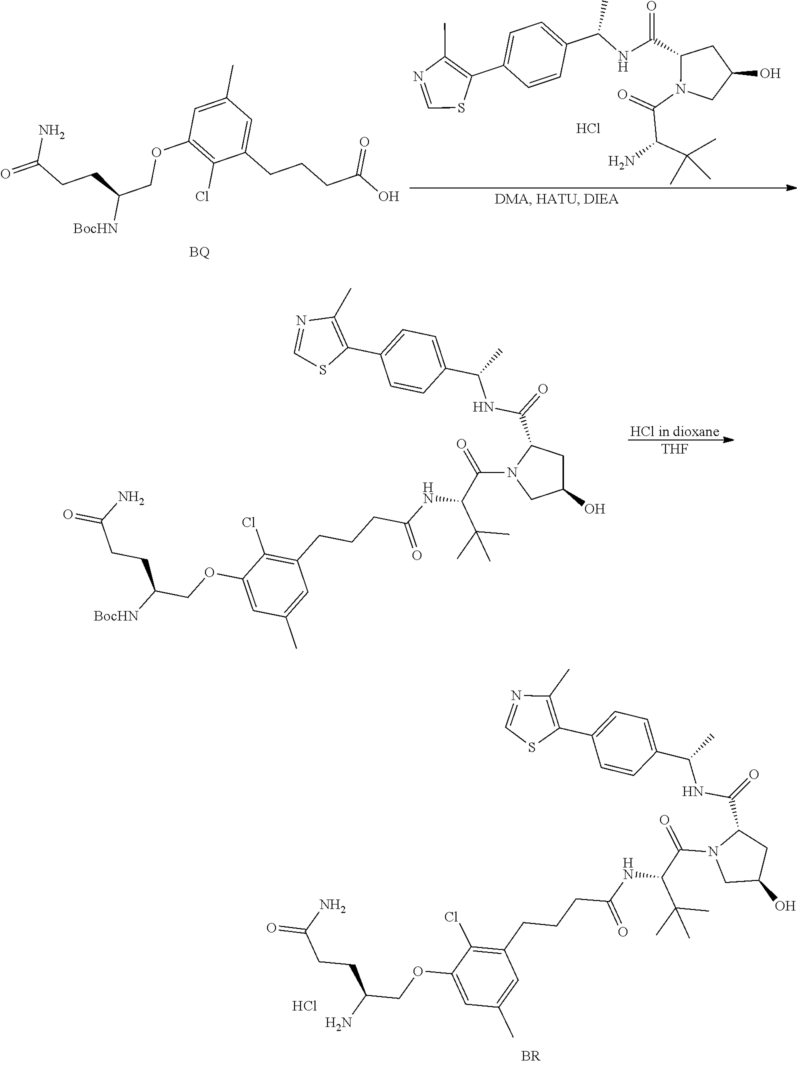 [SMILES: Cc1cc(CCCC(=O)O)c(Cl)c(OC[C@H](CCC(N)=O)NC(=O)OC(C)(C)C)c1 ; Cc1ncsc1-c1ccc([C@H](C)NC(=O)[C@@H]2C[C@@H](O)CN2C(=O)[C@@H](N)C(C)(C)C)cc1 ; Cl ; Cc1cc(CCCC(=O)N[C@H](C(=O)N2C[C@H](O)C[C@H]2C(=O)N[C@@H](C)c2ccc(-c3scnc3C)cc2)C(C)(C)C)c(Cl)c(OC[C@H](CCC(N)=O)NC(=O)OC(C)(C)C)c1 ; Cc1cc(CCCC(=O)N[C@H](C(=O)N2C[C@H](O)C[C@H]2C(=O)N[C@@H](C)c2ccc(-c3scnc3C)cc2)C(C)(C)C)c(Cl)c(OC[C@@H](N)CCC(N)=O)c1 ; Cl]

Step 1—tert-butyl ((S)-5-amino-1-(2-chloro-3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-5-methylphenoxy)-5-oxopentan-2-yl)carbamate. To a stirred mixture of 4-[3-[(2S)-2-[(tert-butoxycarbonyl)amino]-4-carbamoylbutoxy]-2-chloro-5-methylphenyl]butanoic acid (240.00 mg, 0.542 mmol, Intermediate BQ) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (312.78 mg, 0.650 mmol, CAS #1448189-80-7) in DMA (5.00 mL) were added PyBOP (422.95 mg, 0.813 mmol) and TEA (164.48 mg, 1.626 mmol) at rt under air atmosphere. The resulting mixture was stirred for 1 h at rt. On completion, the residue was purified by reverse phase flash chromatography (Column: WelFlash™ C18-I, 20-40 μm, 120 g; Eluent A: Water (plus 10 mmol/L FA); Eluent B: ACN; Gradient: 30%-50% B in 25 min; Flow rate: 60 mL/min; Detector: 220/254 nm; desired fractions were collected at 47% B) and concentrated under reduced pressure to afford the title compound (340 mg, 72% yield) as a white solid. 1H NMR (300 MHz, DMSO-d6) δ 9.00 (s, 1H), 8.39 (d, J=7.8 Hz, 1H), 7.88 (d, J=9.2 Hz, 1H), 7.42 (q, J=8.4 Hz, 4H), 7.28 (s, 1H), 6.87-6.67 (m, 4H), 5.11 (d, J=3.5 Hz, 1H), 4.98-4.88 (m, 1H), 4.55 (d, J=9.3 Hz, 1H), 4.44 (t, J=8.0 Hz, 1H), 4.29 (s, 1H), 3.91 (d, J=6.1 Hz, 2H), 3.82-3.74 (m, 1H), 3.63 (d, J=3.3 Hz, 2H), 2.63 (t, J=7.6 Hz, 2H), 2.47 (s, 3H), 2.36-2.23 (m, 5H), 2.22-2.10 (m, 2H), 2.07-1.97 (m, 1H), 1.93-1.68 (m, 4H), 1.66-1.55 (m, 1H), 1.40-1.37 (m, 12H), 0.96 (s, 9H); LC/MS (ESI, m/z): [(M+H)]+=869.4.

Step 2—(2S,4R)-1-((S)-2-(4-(2-chloro-3-(((S)-2,5-diamino-5-oxopentyl)oxy)-5-methylphenyl)butanamido)-3,3-dimethylbutanoyl)-4-hydroxy-N—((S)-1-(4-(4-methylthiazol-5-yl)phenyl)ethyl)pyrrolidine-2-carboxamide hydrochloride. To a stirred mixture of tert-butyl N-[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate (340.00 mg, 0.391 mmol) in DCM (5.00 mL) was added HCl (gas) in 1,4-dioxane (2.00 mL) dropwise at rt. The resulting mixture was stirred for 1 h at rt. On completion, the resulting mixture was concentrated under reduced pressure to afford the title compound (300 mg, 100% crude yield) as a white solid. 1H NMR (400 MHz, DMSO-d6) δ 9.09 (d, J=1.6 Hz, 1H), 8.46-8.32 (m, 4H), 7.89 (d, J=9.3 Hz, 1H), 7.47-7.41 (m, 2H), 7.39 (d, J=8.4 Hz, 2H), 6.94 (s, 1H), 6.89 (d, J=1.9 Hz, 1H), 6.78 (d, J=1.8 Hz, 1H), 4.95-4.88 (m, 1H), 4.53 (d, J=9.3 Hz, 1H), 4.42 (t, J=8.0 Hz, 1H), 4.30-4.26 (m, 1H), 4.24-4.20 (m, 1H), 4.13 (dd, J=10.5, 5.7 Hz, 1H), 3.62-3.61 (m, 2H), 3.57 (s, 3H), 2.66-2.61 (m, 2H), 2.47 (s, 3H), 2.36-2.23 (m, 5H), 2.22-2.14 (m, 1H), 2.05-1.91 (m, 3H), 1.83-1.73 (m, 3H), 1.38 (d, J=7.0 Hz, 3H), 0.95 (s, 9H); LC/MS (ESI, m/z): [(M+H)]+=769.0.

### 4-[3-[(2S)-2-[(tert-butoxycarbonyl)amino]-4-carbamoylbutoxy]-2,5-difluorophenyl]butanoic acid (Intermediate BS)

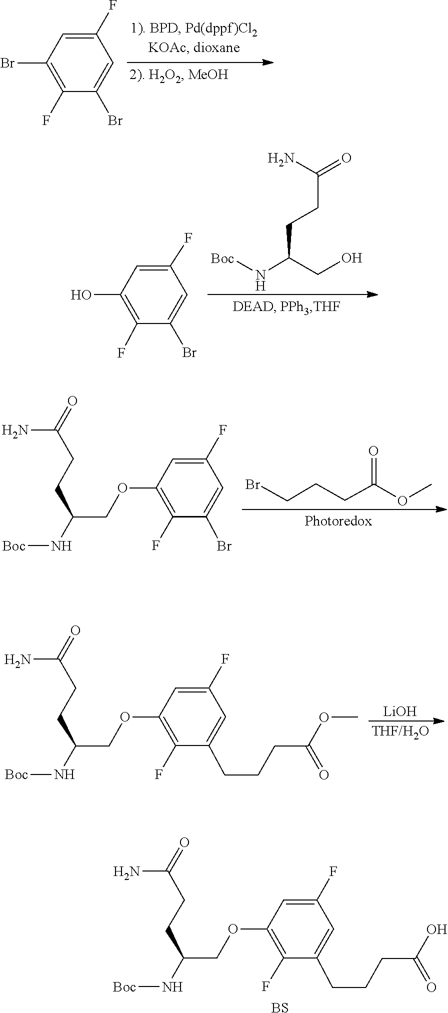

Step 1—3-bromo-2,5-difluorophenol. To a stirred solution of 1,3-dibromo-2,5difluorobenzene (15.00 g, 55.170 mmol) and BPD (14.01 g, 55.17 mmol) in 1,4-dioxane was added KOAc (10.83 g, 110.34 mmol) and Pd(dppf)Cl2 (2.02 g, 2.76 mmol) in portions at rt under nitrogen atmosphere. Then the resulting mixture was stirred for 2 h at 90° C. under nitrogen atmosphere. On completion, the mixture was filtered, and the filter cake was washed with DCM (2×100 mL). The filtrate was concentrated under reduced pressure. The residue was then dissolve d in MeOH (150 mL) and H₂O₂ (30% solution, 15.00 mL) was added at 0° C. The resulting mixture was stirred for 2 h at rt under air atmosphere. On completion, the resulting mixture was concentrated under reduced pressure. The residue was purified by silica gel column chromatography, eluted with PE/EtOAc (10:1,) to afford a yellow oil. The oil was then purified by reverse phase flash chromatography (Column: WelFlash™ C18-I, 330 g; Eluent A: Water (plus 10 mmol/L NH4HCO3); Eluent B: ACN; Gradient: 40%-70% B in 25 min; Flow rate: 85 mL/min; Detector: 220/254 nm; desired fractions were collected at 59% B) and concentrated under reduced pressure to afford the title compound (2 g, 17% yield) as a yellow oil. ¹H NMR (300 MHz, DMSO-d6) δ 10.87 (s, 1H), 7.09-7.02 (m, 1H), 6.85-6.79 (m, 1H).

Step 2—tert-butyl N-[(2S)-1-(3-bromo-2,5-difluorophenoxy)-4-carbamoylbutan-2-yl]carbamate. To a stirred solution of 3-bromo-2,5-difluorophenol (1.50 g, 7.177 mmol) in THF (10.00 mL) was added PPh3 (2.82 g, 10.77 mmol) in portions at rt under nitrogen atmosphere. Then to the mixture was added DEAD (1.87 g, 10.77 mmol) dropwise at 0° C. under nitrogen atmosphere. The resulting mixture was stirred for 15 min at rt under nitrogen atmosphere. Next, tert-butyl N-[(2S)-4-carbamoyl-1-hydroxybutan-2-yl]carbamate (3.33 g, 14.36 mmol) was added and the mixture was stirred for 3 h at rt under nitrogen atmosphere. On completion, the reaction mixture was concentrated under vacuum. Then the crude product was purified by silica gel column chromatography (Mobile Phase A: Petroleum ether, Mobile Phase B: EtOAc; Gradient: 50% B to 100% B in 30 min, 254 nm; the fractions containing the desired product were collected at 100% B) to afford the title compound (530 mg, 18% yield) as a off-white solid. ¹H NMR (300 MHz, DMSO-d6) δ 7.32-7.23 (m, 1H), 7.03-6.92 (m, 1H), 6.90-6.81 (m, 1H), 6.78-6.63 (m, 2H), 4.01-3.86 (m, 2H), 3.78-3.71 (m, 1H), 2.15-2.11 (m, 2H), 1.78-1.72 (m, 1H), 1.61-1.55 (m, 1H), 1.39 (s, 9H); LC/MS (ESI, m/z): [(M+1)]+=423.1, 425.2.

Step 3—Methyl 4-[3-[(2S)-2-[(tert-butoxycarbonyl)amino]-4-carbamoylbutoxy]-2,5-difluorophenyl]butanoate. A mixture of dtbbpy (3.17 mg, 0.012 mmol) and 1,2-dimethoxyethane dihydrochloride nickel (2.60 mg, 0.012 mmol) in DME at rt under nitrogen atmosphere was prepared. Then, to a stirred mixture of tert-butyl N-[(2S)-1-(3-bromo-2,5-difluorophenoxy)-4-carbamoylbutan-2-yl]carbamate (500.00 mg, 1.181 mmol) and methyl 4-bromobutanoate (855.41 mg, 4.73 mmol) in DME (5.00 mL) were added tris(trimethylsilyl)silane (293.75 mg, 1.181 mmol) and Na2CO3 (375.62 mg, 3.54 mmol), IR[DF(CF3)PPY]2(DTBPY)PF6 (13.25 mg, 0.012 mmol) and then the dtbbpy/nickel mixture was added dropwise at rt under nitrogen atmosphere. The reaction mixture was irradiated with ultraviolet lamp for 20 h at rt. On completion, the resulting mixture was concentrated under reduced pressure. The residue was purified by reverse phase flash chromatography (Column: WelFlash™ C18-I, 120 g; EluentA: Water (plus 10 mmol/L NH4HCO3); Eluent B: ACN; Gradient: 30%-60% B in 25 min; Flow rate: 60 mL/min; Detector: 220/254 nm; desired fractions were collected at 45% B) and concentrated under reduced pressure to afford the title compound (280 mg, 53% yield) as a colorless oil. ¹H NMR (300 MHz, DMSO-d6) δ 7.29-7.25 (m, 1H), 7.03-6.93 (m, 1H), 6.88-6.83 (m, 1H), 6.78-6.65 (m, 2H), 3.98-3.83 (m, 2H), 3.77-3.71 (m, 1H), 2.61-2.55 (m, 2H), 2.32-2.27 (m, 4H), 2.11-2.08 (m, 2H), 1.81-1.75 (m, 3H), 1.52-1.47 (m, 2H), 1.39 (s, 9H); LC/MS (ESI, m/z): [(M+1)]+=445.2.

Step 4—4-[3-[(2S)-2-[(tert-butoxycarbonyl)amino]-4-carbamoylbutoxy]-2,5-difluorophenyl]butanoic acid. To a stirred solution of methyl 4-[3-[(2S)-2-[(tert-butoxycarbonyl)amino]-4-carbamoylbutoxy]-2,5-difluorophenyl]butanoate (280.00 mg, 0.630 mmol) in THF (3.00 mL) was added LiOH (150.86 mg, 6.30 0 mmol) in H2O (3.00 mL) at rt under air atmosphere. The resulting mixture was stirred for 2 h at rt under air atmosphere. On completion, the reaction mixture was concentrated undue vacuum. Then, the residue was purified by reverse phase flash chromatography (Column: WelFlash™ C18-I, 20-40 ?m, 120 g; Eluent A: Water (plus 10 mmol/L FA); Eluent B: ACN; Gradient: 20%-60% B in 25 min; Flow rate: 60 mL/min; Detector: 220/254 nm; desired fractions were collected at 46% B) and concentrated under reduced pressure to afford the title compound (250 mg, 92% yield) as a white solid. ¹H NMR (400 MHz, DMSO-d6) δ 7.29-7.24 (m, 1H), 6.99-6.92 (m, 1H), 6.88-6.83 (m, 1H), 6.78-6.72 (m, 1H), 6.71-6.65 (m, 1H), 5.81-5.75 (m, 3H), 3.99-3.92 (m, 2H), 2.60-2.57 (m, 2H), 2.22-2.17 (m, 2H), 2.12-2.08 (m, 2H), 1.77-1.69 (m, 3H), 1.38 (s, 9H); LC/MS (ESI, m/z): [(M+1)]+=431.2.

### (2S,4R)-1-[(2S)-2-(4-[3-[(2S)-2-amino-4-carbamoylbutoxy]-2,5-difluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate BT)

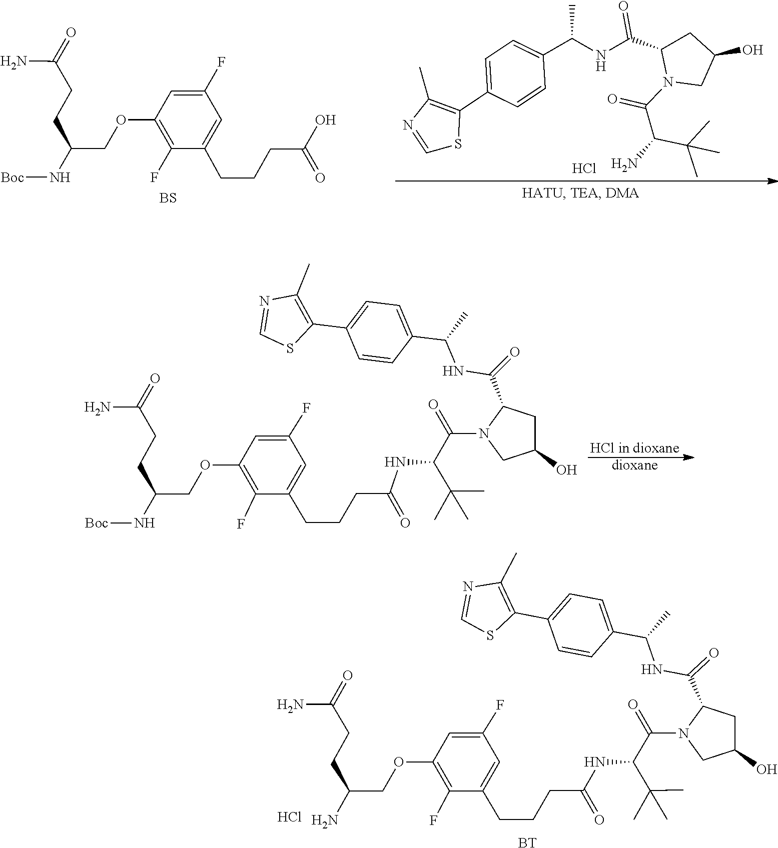

Step 1—tert-butyl N-[(2S)-4-carbamoyl-1-[2,5-difluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate. To a stirred solution of 4-[3-[(2S)-2-[(tert-butoxycarbonyl)amino]-4-carbamoylbutoxy]-2,5-difluorophenyl]butanoic acid (250.00 mg, 0.581 mmol, Intermediate BS) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (335.27 mg, 0.697 mmol, CAS #1448189-80-7) in DMA (4.00 mL) was added TEA (0.323 mL, 2.323 mmol) and HATU (287.08 mg, 0.755 mmol) in portions at rt under nitrogen atmosphere.

The resulting mixture was stirred for 2 h at rt under nitrogen atmosphere. On completion, the reaction was concentrated under vacuum. Next, the residue was purified by reverse phase flash chromatography (Column: WelFlash™ C18-I, 120 g; Eluent A: Water (plus 10 mmol/L NH4HCO3); Eluent B: ACN; Gradient: 20%-70% B in 25 min; Flow rate: 60 mL/min; Detector: 220/254 nm; desired fractions were collected at 46% B) and concentrated under reduced pressure to afford the title compound (390 mg, 78% yield) as a white solid. ¹H NMR (300 MHz, DMSO-d6) δ 9.05-8.99 (m, 1H), 8.44-8.35 (m, 1H), 7.95-7.89 (m, 1H), 7.48-7.41 (m, 4H), 7.31-7.26 (m, 1H), 7.02-6.94 (m, 1H), 6.89-6.82 (m, 1H), 6.79-6.73 (m, 1H), 6.72-6.67 (m, 1H), 5.81-5.76 (m, 1H), 5.16-5.10 (m, 1H), 5.02-4.88 (m, 1H), 4.58-4.51 (m, 1H), 4.48-4.41 (m, 1H), 4.30 (s, 3H), 3.95-3.91 (m, 1H), 3.75-3.72 (m, 1H), 3.63-3.61 (m, 2H), 2.58 (s, 3H), 2.47-2.41 (m, 3H), 2.28-2.25 (m, 3H), 2.17-2.11 (m, 2H), 1.77-1.71 (m, 5H), 1.39 (s, 9H), 0.96 (s, 9H). LC/MS (ESI, m/z): [(M+1)]+=857.3.

Step 2—4R)-1-[(2S)-2-(4-[3-[(2S)-2-amino-4-carbamoylbutoxy]-2,5-difluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2,5-difluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate (390.00 mg, 0.455 mmol) in 1,4-dioxane (4.00 mL) was added HCl (gas) in 1,4-dioxane (4.00 mL, 131.65 mmol) dropwise at rt under air atmosphere.

The resulting mixture was stirred for 2 h at rt under air atmosphere. On completion, the mixture was concentrated under reduced pressure to afford the title compound (360 mg, 100% crude yield). 1H NMR (300 MHz, DMSO-d6) δ 9.08 (s, 1H), 8.40-8.36 (m, 4H), 7.95-7.88 (m, 1H), 7.51-7.44 (m, 3H), 7.46-7.37 (m, 2H), 7.12-7.04 (m, 2H), 6.81-6.75 (m, 1H), 5.41-5.33 (m, 1H), 4.96-4.91 (m, 1H), 4.56-4.51 (m, 1H), 4.48-4.41 (m, 1H), 4.31-4.26 (m, 2H), 4.23-4.17 (m, 2H), 3.61 (s, 3H), 2.61-2.54 (m, 2H), 2.49-2.41 (m, 4H), 2.31-2.29 (m, 2H), 2.26-2.10 (m, 2H), 1.98-1.95 (m, 2H), 1.79-1.72 (m, 3H), 1.39-1.32 (m, 3H), 0.96 (s, 9H); LC/MS (ESI, m/z): [(M+1)]+=757.3.

### 4-[3-[(2S)-2-[(tert-butoxycarbonyl)amino]-4-carbamoylbutoxy]-2-fluoro-5-methylphenyl]butanoic acid (Intermediate BU)

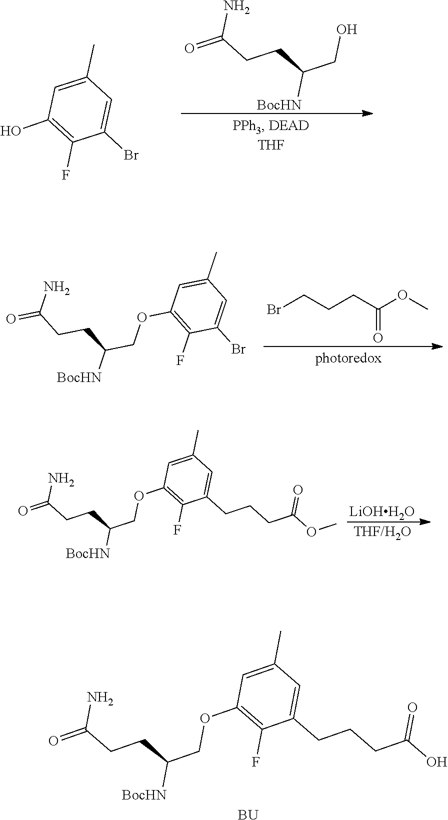

Step 1—Tert-butyl N-[(2S)-1-(3-bromo-2-fluoro-5-methylphenoxy)-4-carbamoylbutan-2-yl]carbamate. To a stirred mixture of tert-butyl N-[(2S)-4-carbamoyl-1-hydroxybutan-2-yl]carbamate (5.00 g, 21.53 mmol) and 3-bromo-2-fluoro-5-methylphenol (4.85 g, 23.68 mmol) in THF (40.00 mL) were added PPh₃ (6.78 g, 25.83 mmol) in portions and DEAD (4.50 g, 25.83 mmol) dropwise at 0° C. under nitrogen atmosphere. The resulting mixture was stirred for 3 h at rt under nitrogen atmosphere. On completion, the reaction mixture was concentrated under vacuum. Next, the residue was purified by silica gel column chromatography, eluted with PE/EtOAc (1:5), to afford the title compound (3.05 g, 34% yield) as a white solid. ¹H NMR (400 MHz, DMSO-d6) δ 7.28 (s, 1H), 7.05 (d, J=6.5 Hz, 2H), 6.86 (d, J=8.5 Hz, 1H), 6.76 (s, 1H), 3.98-3.94 (m, 2H), 3.79-3.67 (m, 1H), 2.28 (s, 3H), 2.16-2.07 (m, 2H), 1.81-1.77 (m, 1H), 1.62-1.58 (m, 1H), 1.39 (s, 9H); LC/MS (ESI, m/z): [(M+1)]+=417.0, 419.0.

Step 2—Methyl 4-[3-[(2S)-2-[(tert-butoxycarbonyl)amino]-4-carbamoylbutoxy]-2-fluoro-5-methylphenyl]butanoate. A mixture of dtbbpy (6.40 mg, 0.024 mmol) in DME (5.00 mL) was added 1,2-dimethoxyethane dihydrochloride nickel (5.24 mg, 0.024 mmol) at 25° C. under nitrogen atmosphere. Then, to a stirred solution of tert-butyl N-[(2S)-1-(3-bromo-2-fluoro-5-methylphenoxy)-4-carbamoylbutan-2-yl]carbamate (1.00 g, 2.39 mmol) and methyl 4-bromobutanoate (1079.38 mg, 5.96 mmol, CAS #4897-84-1) in DME (5.00 mL) were added tris(trimethylsilyl)silane (593.05 mg, 2.39 mmol) and Na₂CO₃ (758.34 mg, 7.16 mmol), IR[DF(CF₃)PPY]₂(DTBPY)PF₆ (26.76 mg, 0.024 mmol) and the nickel catalyst mixture dropwise at 25° C. under nitrogen atmosphere. The final reaction mixture was irradiated with ultraviolet lamp for 20 h at 25° C. On completion, reaction mixture was concentrated under vacuum. Next, the residue was purified by reverse phase flash chromatography (Column: WelFlash™ C18-I, 20-40 um, 330 g; Eluent A: Water (plus 10 mmol/L NH4HCO3); Eluent B: ACN; Gradient: 21%-65% B in 25 min; Flow rate: 80 mL/min; Detector: 254 nm; desired fractions were collected at 47% B) and concentrated under reduced pressure to afford the title compound (817 mg, 78%) as a white solid. ¹H NMR (400 MHz, DMSO-d6) δ 7.27 (s, 1H), 6.87-6.79 (m, 2H), 6.75 (s, 1H), 6.63-6.59 (m, 1H), 3.94-3.87 (m, 2H), 3.75-3.69 (m, 1H), 3.59 (s, 3H), 2.58-2.54 (m, 2H), 2.36-2.27 (m, 3H), 2.24 (s, 3H), 2.18-2.08 (m, 2H), 1.81-1.77 (m, 3H), 1.39 (s, 9H); LC/MS (ESI, m/z): [(M+1)]+=441.2.

Step 3—4-[3-[(2S)-2-[(tert-butoxycarbonyl)amino]-4-carbamoylbutoxy]-2-fluoro-5-methylphenyl]butanoic acid. To a stirred solution of methyl 4-[3-[(2S)-2-[(tert-butoxycarbonyl)amino]-4-carbamoylbutoxy]-2-fluoro-5-methylphenyl]butanoate (817.00 mg, 1.86 mmol) in THF (15.00 mL) was added LiOH (444.15 mg, 18.55 mmol) in H₂O (15.00 mL) dropwise at rt under air atmosphere. The resulting mixture was stirred for 3 h at rt under air atmosphere. On completion, the mixture was acidified to pH 4 with conc. HCl. The resulting mixture was extracted with CH2Cl2 (3×20 mL). The combined organic layers were washed with brine (2×20 mL), and dried over anhydrous Na2SO4. After filtration, the filtrate was concentrated under reduced pressure to afford the title compound (779.2 mg, 99%) as a white solid. ¹H NMR (400 MHz, DMSO-d6) δ 12.00 (s, 1H), 7.27 (s, 1H), 6.83 (t, J=7.4 Hz, 2H), 6.74 (s, 1H), 6.64-6.58 (m, 1H), 3.92-3.85 (m, 1H), 3.78-3.67 (m, 1H), 2.58-2.53 (m, 2H), 2.23 (s, 3H), 2.22-2.13 (m, 2H), 2.13-2.09 (m, 2H), 1.87-1.69 (m, 3H), 1.61-1.57 (m, 1H), 1.50-1.46 (m, 1H), 1.39 (s, 9H); LC/MS (ESI, m/z): [(M+1)]+=427.1.

### (2S,4R)-1-[(2S)-2-(4-[3-[(2S)-2-amino-4-carbamoylbutoxy]-2-fluoro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate BV)

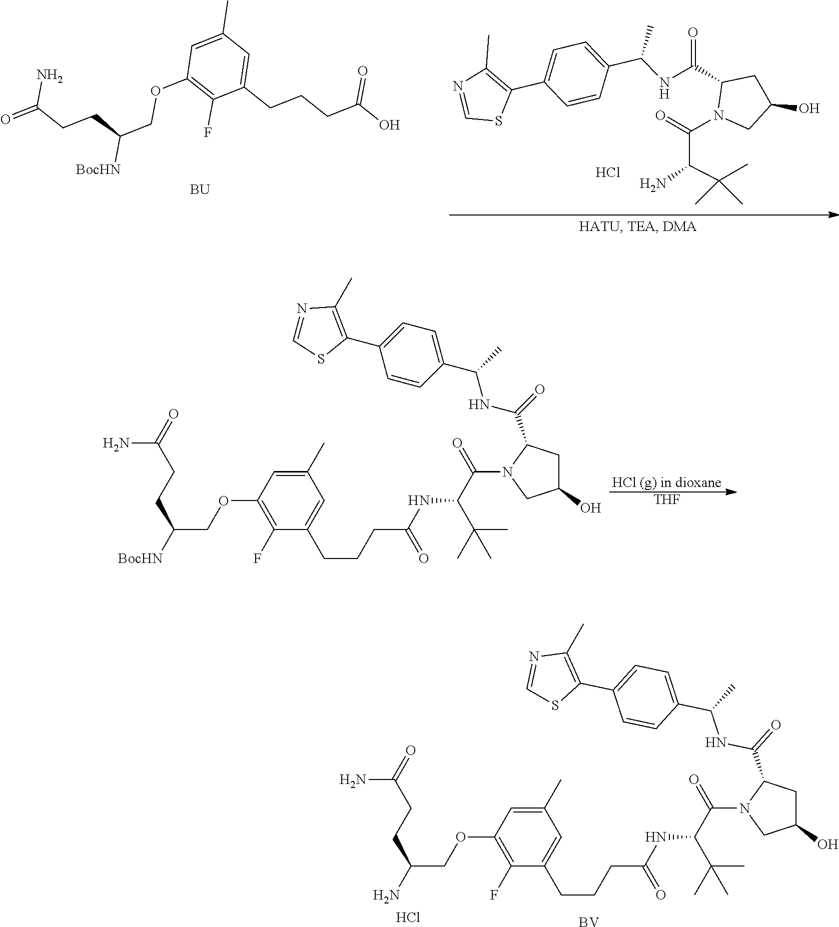

Step 1—tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate. To a stirred solution of (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (783.96 mg, 1.63 mmol) and 4-[3-[(2S)-2-[(tert-butoxycarbonyl)amino]-4-carbamoylbutoxy]-2-fluoro-5-methylphenyl]butanoic acid (579.20 mg, 1.36 mmol, Intermediate BU) in DMA (5.00 mL) were added HATU (619.66 mg, 1.63 mmol) in portions and TEA (0.566 mL, 4.074 mmol) dropwise at rt under nitrogen atmosphere. The resulting mixture was stirred for 16 h at rt under nitrogen atmosphere. On completion, the reaction mixture was purified directly by reverse phasefFlash chromatography (Column: WelFlash™ C18-I, 20-40 um, 330 g; Eluent A: Water (plus 10 mmol/L NH₄HCO₃); Eluent B: ACN; Gradient: 35%-70% B in 25 min; Flow rate: 60 mL/min; Detector: 220 nm; desired fractions were collected at 60% B) and concentrated under reduced pressure to afford the title compound (754 mg, 65% yield) as a white solid. ¹H NMR (400 MHz, DMSO-d6) δ 8.99 (s, 1H), 8.38 (d, J=7.7 Hz, 1H), 7.86 (d, J=9.2 Hz, 1H), 7.47-7.39 (m, 2H), 7.41-7.35 (m, 2H), 7.28-7.24 (m, 1H), 6.82 (t, J=8.4 Hz, 2H), 6.74 (s, 1H), 6.62-6.57 (m, 1H), 5.12-5.08 (m, 1H), 4.97-4.87 (m, 1H), 4.53 (d, J=9.3 Hz, 1H), 4.43 (t, J=8.0 Hz, 1H), 4.32-4.24 (m, 1H), 3.89 (d, J=6.0 Hz, 2H), 3.75-3.67 (m, 1H), 3.64-3.59 (m, 2H), 3.18-3.15 (m, 1H), 2.46 (s, 3H), 2.34-2.26 (m, 1H), 2.24 (s, 3H), 2.21-2.04 (m, 3H), 2.06-1.96 (m, 1H), 1.85-1.69 (m, 4H), 1.63-1.52 (m, 1H), 1.39 (s, 9H), 1.38-1.32 (m, 3H), 1.29-1.23 (m, 1H), 0.95 (s, 9H); LC/MS (ESI, m/z): [(M+1)]+=853.4.

Step 2—(2S,4R)-1-[(2S)-2-(4-[3-[(2S)-2-amino-4-carbamoylbutoxy]-2-fluoro-5-methylphenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)-5-methylphenoxy]butan-2-yl]carbamate (754.00 mg, 0.88 mmol) in THF (5.00 mL) was added HCl (gas) in 1,4-dioxane (5.00 mL) dropwise at rt under nitrogen atmosphere. The resulting mixture was stirred for 3 h at rt under nitrogen atmosphere. On completion, the reaction mixture was concentrated under reduced pressure to afford the title compound (695 mg, 100% crude yield) as a white solid. ¹H NMR (300 MHz, DMSO-d6) δ 9.08 (s, 1H), 8.45-8.40 (m, 1H), 8.37-8.33 (m, 1H), 7.88 (d, J=9.1 Hz, 1H), 7.51-7.39 (m, 4H), 6.99-6.86 (m, 2H), 6.73-6.62 (m, 1H), 5.03-4.90 (m, 1H), 4.54 (d, J=8.9 Hz, 1H), 4.44 (t, J=8.2 Hz, 1H), 4.34-4.27 (m, 1H), 4.27-4.17 (m, 1H), 4.16-4.10 (m, 1H), 3.50-3.32 (m, 4H), 3.21-3.14 (m, 2H), 2.49-2.46 (m, 3H), 2.38-2.30 (m, 3H), 2.29-2.24 (m, 4H), 2.12-1.98 (m, 1H), 1.96-1.87 (m, 2H), 1.86-1.71 (m, 4H), 1.39 (d, J=6.9 Hz, 3H), 1.31-1.22 (m, 1H), 0.96 (s, 9H); LC/MS (ESI, m/z): [(M+1)]+=753.3.

### 4-[3-[(2S)-2-[(tert-butoxycarbonyl)amino]-4-carbamoylbutoxy]-2-chloro-5-fluorophenyl]butanoic acid (Intermediate BW)

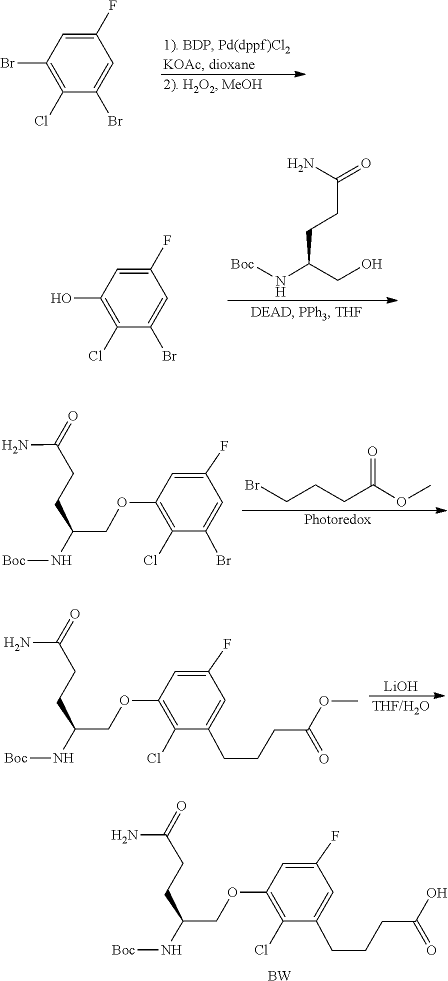

Step 1—3-bromo-2-chloro-5-fluorophenol. To a stirred solution of 1,3-dibromo-2-chloro-5-fluorobenzene (15.00 g, 52.022 mmol) and BPD (13.21 g, 52.02 mmol) in dioxane (150.00 mL) were added KOAc (10.21 g, 104.04 mmol) and Pd(dppf)Cl₂ (1.90 g, 2.60 mmol) in portions at rt under air atmosphere. The resulting mixture was stirred for 2 h at 90° C. under nitrogen atmosphere. On completion, the resulting mixture was filtered, the filter cake was washed with DCM (3×30 mL). The filtrate was then concentrated under reduced pressure. Next, to the mixture was added MeOH (150.00 mL) and H₂O₂ (15.00 mL) in portions, then the resulting mixture was stirred for 16 h at rt under air atmosphere. On completion, the reaction mixture was concentrated under vacuum. Next, the residue was purified by silica gel column chromatography, eluted with PE/EtOAc (5:1) to afford the title compound (1.28 g, 11% yield) as a yellow oil. ¹H NMR (300 MHz, DMSO-d6) δ 11.20 (s, 1H), 7.21-7.19 (m, 1H), 6.8-6.72 (m, 1H); LC/MS (ESI, m/z): [(M+1)]+=225.1.

Step 2—Tert-butyl N-[(2S)-1-(3-bromo-2-chloro-5-fluorophenoxy)-4-carbamoylbutan-2-yl]carbamate. To a stirred solution of 3-bromo-2-chloro-5-fluorophenol (2.52 g, 11.19 mmol) in THF (30.00 mL) were added PPh₃ (4.40 g, 16.79 mmol) in portions at 0° C. under nitrogen atmosphere. To the above mixture was added DEAD (2.92 g, 16.79 mmol) dropwise. The reaction mixture was stirred for 15 min at rt. To the above mixture was added tert-butyl N-[(2S)-4-carbamoyl-1-hydroxybutan-2-yl]carbamate (2.60 g, 11.19 mmol) in portions at rt. The resulting mixture was stirred for 3 h at rt under nitrogen atmosphere. On completion, the reaction mixture was diluted with water (50 mL) and extracted with EtOAc (3×50 mL). The combined organic layers was washed with brine (3×50 mL), dried with Na2SO4 and filtered. The filtrate was concentrated under vacuum. Next, the crude product was purified by silica gel column chromatography with the following conditions (Mobile Phase A: PE, Mobile Phase B: EA; Gradient: 50% B to 100% B in 30 min, 254 nm. The fractions containing the desired product were collected at 78% B to afford tert-butyl N-[(2S)-1-(3-bromo-2-chloro-5-fluorophenoxy)-4-carbamoylbutan-2-yl]carbamate (800 mg, 16.25%) as a white solid. ¹H NMR (300 MHz, DMSO-d6) δ 7.60-7.50 (m, 1H), 7.36-7.23 (m, 2H), 6.90-6.73 (m, 2H), 4.12-3.92 (m, 2H), 3.79 (s, 1H), 2.26-2.07 (m, 2H), 1.90-1.70 (m, 1H), 1.65-1.60 (m, 1H), 1.40 (s, 9H); LC/MS (ESI, m/z): [(M+1)]+=439.0, 441.0.

Step 3—Methyl 4-[3-[(2S)-2-[(tert-butoxycarbonyl)amino]-4-carbamoylbutoxy]-2-chloro-5-fluorophenyl]butanoate. To a mixture of dtbbpy (3.05 mg, 0.011 mmol) in DCM (2.5 mL) was added 1,2-dimethoxyethane dihydrochloride nickel (2.50 mg, 0.011 mmol) at 25° C. under nitrogen atmosphere. Then, to a mixture of tert-butyl N-[(2S)-1-(3-bromo-2-chloro-5-fluorophenoxy)-4-carbamoylbutan-2-yl]carbamate (500.00 mg, 1.14 mmol) and methyl 4-bromobutanoate (823.40 mg, 4.55 mmol) in DCM (2.50 mL) were added tris(trimethylsilyl)silane (282.76 mg, 1.14 mmol), Na₂CO₃ (361.56 mg, 3.41 mmol), IR[DF(CF₃)PPY]₂(DTBPY)PF (12.76 mg, 0.011 mmol) and the nickel catalyst solution dropwise at 25° C. under nitrogen atmosphere. The final reaction mixture was irradiated with ultraviolet lamp for 16 h at 25° C. On completion, the resulting mixture was concentrated under reduced pressure. The residue was purified by reverse phase Flash chromatography (Column: WelFlash™ C18-I, 20-40 um, 120 g; Eluent A: Water (plus 10 mmol/L NH4HCO3); Eluent B: ACN; Gradient: 50%-65% B in 25 min; Flow rate: 80 mL/min; Detector: 220/254 nm; desired fractions were collected at 55% B) and concentrated under reduced pressure to afford the title compound (170 mg, 32% yield) as an black solid. ¹H NMR (300 MHz, DMSO-d6) δ 7.28 (s, 1H), 6.99-6.90 (m, 1H), 6.89-6.69 (m, 3H), 4.05-3.89 (m, 2H), 3.84-3.71 (m, 1H), 3.36-3.59 (m, 1H), 2.77-2.68 (m, 2H), 2.41-2.26 (m, 3H), 2.24-2.08 (m, 2H), 1.90-1.75 (m, 3H), 1.70-1.47 (m, 2H), 1.39 (s, 9H); LC/MS (ESI, m/z): [(M+1)]+=461.2.

Step 4—4-[3-[(2S)-2-[(tert-butoxycarbonyl)amino]-4-carbamoylbutoxy]-2-chloro-5-fluorophenyl]butanoic acid. To a stirred solution of methyl 4-[3-[(2S)-2-[(tert-butoxycarbonyl)amino]-4-carbamoylbutoxy]-2-chloro-5-fluorophenyl]butanoate (170.00 mg, 0.37 mmol) in THF (2.00 mL) were added LiOH (88.32 mg, 3.69 m mol) and H₂O (2.00 mL) in portions at rt under air atmosphere. The resulting mixture was stirred for 2 h at rt under air atmosphere. On completion, the resulting mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 80 mL/min; Detector: 220/254 nm; desired fractions were collected at 48% B) and concentrated under reduced pressure to afford the title compound (120 mg, 73% yield) as a white solid. ¹H NMR (300 MHz, DMSO-d6) δ 12.10 (s, 1H), 7.28 (s, 1H), 6.87-6.72 (m, 3H), 4.10-4.28 (m, 2H), 3.95 (m, 2H), 2.74-2.69 m, 2H), 2.28-2.20 (m, 2H), 2.17-2.13 (m, 2H), 1.82-1.75 (m, 3H), 1.65-1.60 (m, 1H), 1.40 (s, 9H); LC/MS (ESI, m/z): [(M+1)]+=447.1.

### (2SAR)-1-[(2S)-2-(4-[3-[(2S)-2-amino-4-carbamoylbutoxy]-2-chloro-5-fluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate BX)

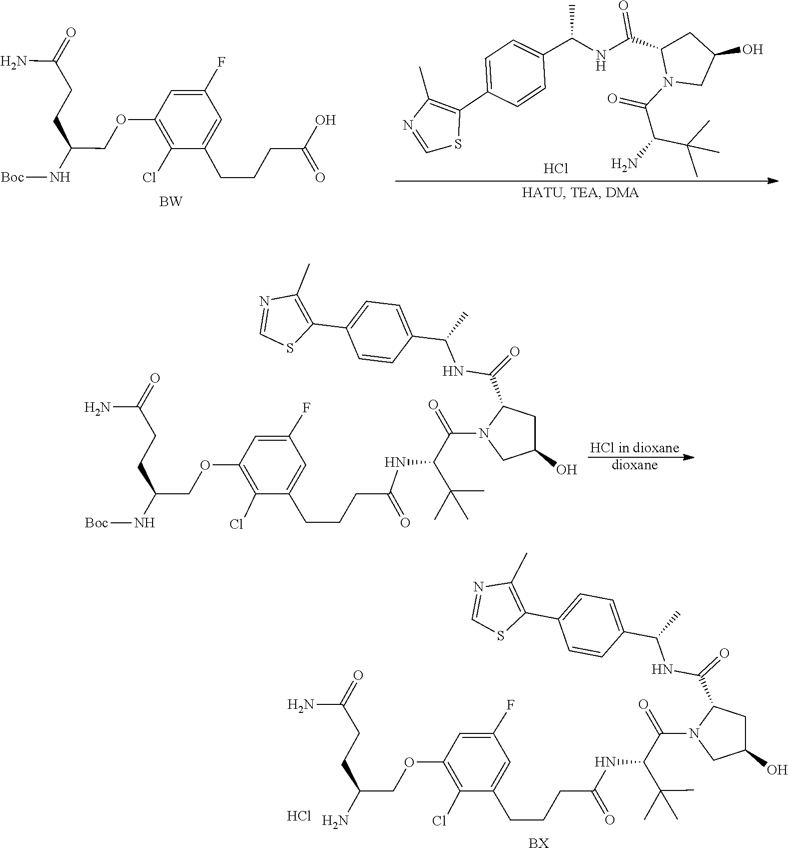

Step 1—Tert-butyl N-[(2S)-4-carbamoyl-1-[2-chloro-5-fluoro-3-(3-[I(2S)-1-[(2S,4R)-4-hydroxy-2-[[(1 S)-1-[4-(4-methyl-1,3-thiazol-5-yl)phenyl] ethyl] carbamoyl]pyrrolidin-1-yl]-3,3-dimethyl-1-oxobutan-2-yl]carbamoyl]propyl)phenoxy]butan-2-yl]carbamate. To a stirred solution of 4-[3-[(2S)-2-[(tert-butoxycarbonyl)amino]-4-carbamoylbutoxy]-2-chloro-5-fluorophenyl]butanoic acid (120.00 mg, 0.27 mmol, Intermediate BW) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (155.00 mg, 0.32 mmol) in DMA (2.00 mL) were added TEA (0.149, 1.08 m mol) dropwise and HATU (132.73 mg, 0.35 mmol) in portions at rt under air atmosphere. The resulting mixture was stirred for 2 h at rt under air atmosphere. On completion, the resulting mixture was concentrated under reduced pressure. The residue was purified by reverse phase flash chromatography (Column: WelFlash™ C18-I, 20-40 um, 120 g; Eluent A: Water (plus 10 mmol/L NH₄CO₃); Eluent B: ACN; Gradient: 30%-50% B in 25 min; Flow rate: 80 mL/min; Detector: 220/254 nm; desired fractions were collected at 47% B) and concentrated under reduced pressure to afford the title compound (230 mg, 98% yield) as a white solid. ¹H NMR (300 MHz, DMSO-d6) δ 9.00 (s, 1H), 8.39 (d, J=7.8 Hz, 1H), 7.91 (d, J=9.3 Hz, 1H), 7.42-7.78 (m, 4H), 7.30-7.26 (m, 1H), 6.99-6.95 (m, 1H), 6.87-6.73 (m, 3H), 5.13-5.10 (m, 1H), 4.99-4.90 (m, 1H), 4.54 (d, J=9.3 Hz, 1H), 4.44 (t, J=8.0 Hz, 1H), 4.30-4.28 (m, 1H), 4.01-3.91 (m, 2H), 3.79-3.75 (m, 1H), 2.69-2.65 (m, 2H), 2.47 (s, 3H), 2.30-2.16 (m, 4H), 2.07-1.95 (m, 1H), 1.89-1.79 (m, 6H), 1.65-1.62 (m, 1H), 1.39 (s, 9H), 1.34-1.32 (m, 3H), 0.96 (s, 9H); LC/MS (ESI, m/z): [(M+H)]+=873.3.

Step 2—(2S,4R)-1-[(2S)-2-(4-[3-[(2S)-2-amino-4-carbamoylbutoxy]-2-chloro-5-fluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution oftert-butyl N-[(1S)-3-carbamoyl-1-[[2-chloro-3-(5-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]pentyl)phenyl]carbamoyl]propyl]carbamate (610.00 mg, 0.68 mmol) in dioxane (6.00 mL) was added HCl (gas) in 1,4-dioxane (6.00 mL, 197.47 mmol) dropwise at rt under air atmosphere. The resulting mixture was stirred for 2 h at rt under air atmosphere. On completion, the resulting mixture was concentrated under reduced pressure to afford the title compound (550 mg, 97% yield) as a yellow solid. ¹H NMR (300 MHz, DMSO-d6) δ 9.08 (s, 1H), 8.47-8.30 (m, 4H), 7.91 (d, J=9.2 Hz, 1H), 7.48-7.38 (m, 4H), 7.12-7.06 (m, 1H), 7.03 (s, 1H), 6.90-6.85 (m, 1H), 4.94-4.90 (m, 1H), 4.56-4.51 (m, 1H), 4.46-4.41 (m, 1H), 4.35-4.24 (m, 2H), 4.19-3.77 (m, 2H), 3.60-3.57 (m, 2H), 2.73-2.70 (m, 2H), 2.48 (s, 3H), 2.34-2.30 (m, 3H), 2.19-1.98 (m, 4H), 1.87-1.71 (m, 3H), 1.39 (d, J=7.0 Hz, 3H), 1.26-1.24 (m, 1H), 0.96 (s, 9H); LC/MS (ESI, m/z): [(M+1-56)]+=773.3.

### (1R,4R)-4-[2-[1-(2,6-dioxopiperidin-3-yl)-3-methyl-2-oxo-1,3-benzodiazol-5-yl]ethyl]cyclohexane-1-carboxylic acid (Intermediate BY)

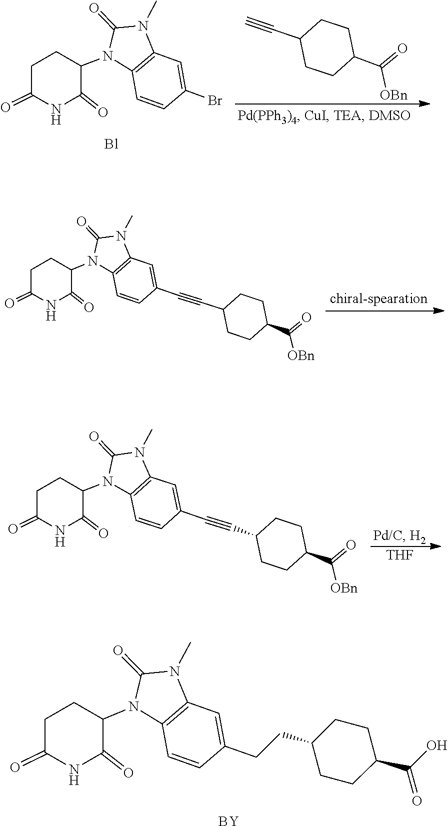

Step 1—Benzyl 4-[2-[1-(2,6-dioxopiperidin-3-yl)-3-methyl-2-oxo-1,3-benzodiazol-5-yl]ethynyl]cyclohexane-1-carboxylate. To a solution of 3-(5-bromo-3-methyl-2-oxo-1,3-benzodiazol-1-yl)piperidine-2,6-dione (2.7 g, 7.98 mmol, Intermediate BI) and benzyl 4-ethynylcyclohexane-1-carboxylate (2.13 g, 8.78 mmol, CAS #1254116-40-9) in DMSO (10.00 mL) were added Pd(PPh3)4 (920 mg, 0.798 mmol), CuI (150 mg, 0.798 mmol) and TEA (5 mL) at 25° C. The mixture was stirred at 85° C. for 2 h. On completion, the reaction mixture was diluted with water (50 mL) and extracted with EtOAc (3×50 mL). The combined organic layers was washed with brine (5×50 mL), dried with Na2SO4 and filtered. The filtrate was concentrated under vacuum. The crude product was purified by silica gel column chromatography (Mobile Phase A: PE, Mobile Phase B: EA; Gradient: 50% B to 100% B in 30 min, 254 nm; the fractions containing the desired product were collected at 50% B) to afford the title compound (1.02 g, 26% yield) as a yellow solid. ¹H NMR (400 MHz, Chloroform-d) δ 8.68 (s, 1H), 7.42-7.30 (m, 5H), 7.15-7.11 (m, 1H), 7.09-7.05 (m, 1H), 6.74 (d, J=8.2 Hz, 1H), 5.21 (dd, J=12.7, 5.3 Hz, 1H), 5.14 (d, J=4.7 Hz, 2H), 3.42 (s, 3H), 2.96-2.80 (m, 2H), 2.75-2.68 (m, 1H), 2.56-2.31 (m, 2H), 2.25-2.18 (m, 1H), 2.15-2.00 (m, 4H), 1.59-1.38 (m, 4H). LC/MS (ESI, m/z): [(M+1)]+=500.2.

Step 2—Benzyl (1R,4R)-4-[2-[1-(2,6-dioxopiperidin-3-yl)-3-methyl-2-oxo-1,3-benzodiazol-5-yl]ethynyl]cyclohexane-1-carboxylate. Benzyl 4-[2-[1-(2,6-dioxopiperidin-3-yl)-3-methyl-2-oxo-1,3-benzodiazol-5-yl]ethynyl]cyclohexane-1-carboxylate (1.00 g) was separated by SFC with the following conditions: Column: CHIRALPAK IH, 2.0*25 cm, 5 um; Mobile Phase A: CO2, Mobile Phase B: MeOH (0.1% HAC); Flow rate: 60 mL/min; Gradient: 40% B; 220 nm; RTL: 5.15; RT2: 6.29). This resulted in benzyl (1R,4R)-4-[2-[1-(2,6-dioxopiperidin-3-yl)-3-methyl-2-oxo-1,3-benzodiazol-5-yl]ethynyl]cyclohexane-1-carboxylate (700 mg, 70% yield) as a light yellow solid and benzyl (1S,Ss)-4-[2-[1-(2,6-dioxopiperidin-3-yl)-3-methyl-2-oxo-1,3-benzodiazol-5-yl]ethynyl]cyclohexane-1-carboxylate (100 mg, 10% yield) as a light yellow solid. ¹H NMR (400 MHz, Chloroform-d) δ 8.15 (s, 1H), 7.44-7.32 (m, 5H), 7.19-7.13 (m, 1H), 7.11-7.05 (m, 1H), 6.77-6.71 (m, 1H), 5.25-5.19 (m, 1H), 5.18-5.12 (m, 2H), 3.43 (s, 3H), 3.02-2.92 (m, 1H), 2.78-2.75 (m, 2H), 2.55-2.45 (m, 1H), 2.45-2.34 (m, 1H), 2.30-2.21 (m, 1H), 2.17-2.05 (m, 3H), 1.58-1.52 (m, 4H); LC/MS (ESI, m/z): [(M+1)]+=500.2.

Step 3—(1R,4R)-4-[2-[1-(2,6-dioxopiperidin-3-yl)-3-methyl-2-oxo-1,3-benzodiazol-5-yl]ethyl]cyclohexane-1-carboxylic acid. To a solution of benzyl (1r,4r)-4-[2-[1-(2,6-dioxopiperidin-3-yl)-3-methyl-2-oxo-1,3-benzodiazol-5-yl]ethynyl]cyclohexane-1-carboxylate (700 mg, 1.40 mmol) in THF (10.00 mL) was added Pd/C (2.98 mg, 0.028 mmol) under nitrogen atmosphere. The mixture was hydrogenated at rt for 2 h under hydrogen atmosphere using a hydrogen balloon. On completion, the reaction was filtered through a celite pad and concentrated under reduced pressure to give the title compound (350 mg, 54% yield) as a light yellow solid. ¹H NMR (300 MHz, DMSO-d6) δ 12.02 (s, 1H), 11.11 (s, 1H), 7.09-6.98 (m, 2H), 6.91-6.87 (m, 1H), 5.81-5.75 (m, 1H), 5.38-5.30 (m, 1H), 2.92-2.88 (m, 1H), 2.85-2.57 (m, 4H), 2.33-1.98 (m, 3H), 1.87-1.85 (m, 4H), 1.50-1.44 (m, 3H), 1.29-1.22 (m, 3H), 0.97-0.91 (m, 2H); LC/MS (ESI, m/z): [(M+1)]+=414.3.

### Tert-butyl N-[(5S,8S,10aR)-8-[[(1S)-3-carbamoyl-1-[[(4-methanesulfonylphenyl)methyl]carbamoyl]propyl]carbamoyl]-6-oxo-octahydro-1H-pyrrolo[1,2-a][1,5]diazocin-5-yl]carbamate (Intermediate BZ)

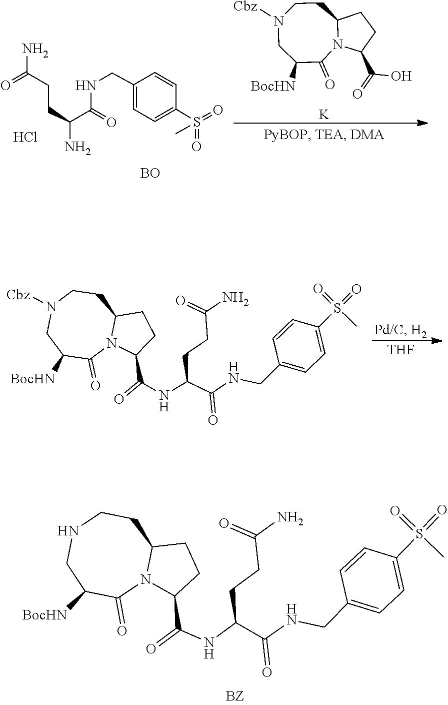

Step 1—Benzyl (5S,8S,10aR)-5-[(tert-butoxycarbonyl)amino]-8-[(3-carbamoyl-1-[[(4-methanesulfonylphenyl)methyl]carbamoyl]propyl)carbamoyl]-6-oxo-octahydropyrrolo[1,2-a][1,5]diazocine-3-carboxylate. To a stirred solution of (5S,8S,10aR)-3-[(benzyloxy)carbonyl]-5-[(tert-butoxycarbonyl)amino]-6-oxo-octahydropyrrolo[1,2-a][1,5]diazocine-8-carboxylic acid (3.40 g, 7.37 mmol, Intermediate K) and (2S)-2-amino-N-[(4-methanesulfonylphenyl)methyl]pentanediamide hydrochloride (2.58 g, 7.38 mmol, Intermediate BO) in DMA (25.00 mL) were added TEA (3.08 mL, 22.14 mmol) and PyBOP (4.60 g, 8.84 mmol) dropwise at rt under nitrogen atmosphere. The resulting mixture was stirred for 1 h at rt under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 35%-60% B in 25 min; Flow rate: 80 mL/min; Detector: 220/254 nm; desired fractions were collected at 50% B) and concentrated under reduced pressure to afford the title compound (4 g, 72% yield) as a white solid. ¹H NMR (300 MHz, DMSO-d6) δ 8.53 (t, J=6.0 Hz, 1H), 8.27 (d, J=7.6 Hz, 1H), 7.96-7.83 (m, 2H), 7.55-7.49 (m, 2H), 7.47-7.28 (m, 5H), 7.28-7.22 (m, 1H), 6.97-6.91 (m, 1H), 6.76 (d, J=5.2 Hz, 1H), 5.13 (s, 2H), 4.45-4.38 (m, 4H), 4.30-4.08 (m, 1H), 3.84-3.78 (m, 1H), 3.63-3.54 (m, 1H), 3.43-3.37 (m, 1H), 3.19 (s, 3H), 2.27-2.12 (m, 2H), 2.14-2.05 (m, 4H), 2.04-1.56 (m, 6H), 1.40 (s, 9H); LC/MS (ESI, m/z): [(M+1)]+=757.3.

Step 2—Tert-butyl N-[(5S,8S,10aR)-8-[(3-carbamoyl-1-[[(4-methanesulfonylphenyl)methyl]carbamoyl]propyl)carbamoyl]-6-oxo-octahydro-1H-pyrrolo[1,2-a][1,5]diazocin-5-yl]carbamate. To a solution of benzyl (5S,8S,10aR)-5-[(tert-butoxycarbonyl)amino]-8-[(3-carbamoyl-1-[[(4-methanesulfonylphenyl)methyl]carbamoyl]propyl)carbamoyl]-6-oxo-octahydropyrrolo[1,2-a][1,5]diazocine-3-carboxylate (740.00 mg, 0.978 mmol) in THF (12.00 mL) was added Pd/C (104.05 mg, 0.098 mmol, 10 wt %) under nitrogen atmosphere. The mixture was hydrogenated at rt for 6 h under hydrogen atmosphere using a hydrogen balloon. On completion, the reaction mixture was filtered through a celite pad and concentrated under reduced pressure. The residue was purified by reverse phase flash chromatography (Column: WelFlash™ C18-I, 20-40 um, 330 g; EluentA: Water (plus 10 mmol/L FA); Eluent B: ACN; Gradient: 35%-60% B in 25 min; Flow rate: 80 mL/min; Detector: 220/254 nm; desired fractions were collected at 50% B) and concentrated under reduced pressure to afford the title compound (500 mg, 87% yield) as a white solid. ¹H NMR (300 MHz, Chloroform-d) δ 9.04 (d, J=7.6 Hz, 1H), 7.98-7.80 (m, 3H), 7.48 (d, J=8.3 Hz, 2H), 6.51 (s, 1H), 5.66 (s, 1H), 5.18 (d, J=8.2 Hz, 1H), 4.93-4.86 (m, 1H), 4.61-4.19 (m, 5H), 3.43-3.35 (m, 1H), 3.17-3.09 (m, 1H), 3.02 (s, 3H), 2.76-2.71 (m, 1H), 2.63-2.28 (m, 2H), 2.28-1.98 (m, 2H), 1.61-1.48 (m, 1H), 1.44 (s, 9H); LC/MS (ESI, m/z): [(M+1)]+=623.2.

### (2S)-1-[2-[(tert-butoxycarbonyl)amino]acetyl]pyrrolidine-2-carboxylic acid (Intermediate CA)

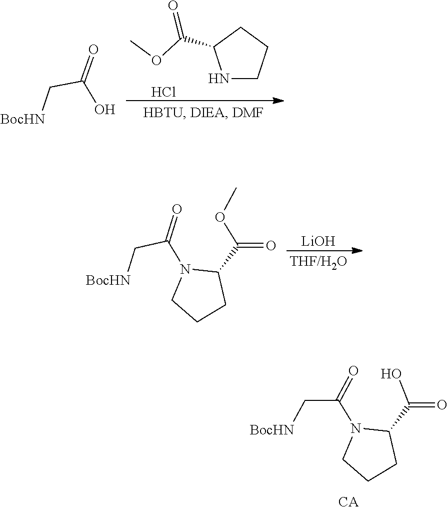

Step 1—Methyl (2S)-1-[2-[(tert-butoxycarbonyl)amino]acetyl]pyrrolidine-2-carboxylate. To a stirred solution of [(tert-butoxycarbonyl)amino]acetic acid (5.00 g, 28.54 mmol) and methyl (2S)-pyrrolidine-2-carboxylate hydrochloride (5.67 g, 34.25 mmol) in DMF (50.00 mL) were added DIEA (18.87 mL, 114.17 mmol) and HBTU (14.07 g, 37.10 mmol) in portions at rt under air atmosphere. The resulting mixture was stirred for 2 h at rt under air atmosphere. On completion, the resulting mixture was extracted with EtOAc (3×50 mL). The combined organic layers were washed with brine (1×30 mL), and dried over anhydrous Na2SO4. After filtration, the filtrate was concentrated under reduced pressure. The residue was purified by reverse phase flash chromatography (Column: WelFlash™ C18-I, 20-40 um, 330 g; Eluent A: Water (plus 0.1% FA); Eluent B: ACN; Gradient: 35%-55% B in 25 min; Flow rate: 60 mL/min; Detector: 220/254 nm; desired fractions were collected at 45% B) and concentrated under reduced pressure to afford the title compound (7.6 g, 93% yield) as a yellow oil. 1H NMR (300 MHz, DMSO-d6) δ 6.91-6.85 (m, 1H), 4.40-4.32 (m, 1H), 3.91-3.83 (m, 1H), 3.75-3.66 (m, 1H), 3.68-3.62 (m, 2H), 3.58-3.48 (m, 2H), 3.46-3.37 (m, 1H), 2.24-2.06 (m, 1H), 2.03-1.79 (m, 3H), 1.44 (s, 9H); LC/MS (ESI, m/z): [(M+H)]+=287.2.

Step 2—(2S)-1-[2-[(tert-butoxycarbonyl)amino]acetyl]pyrrolidine-2-carboxylic acid. To a stirred solution of methyl (2S)-1-[2-[(tert-butoxycarbonyl)amino]acetyl]pyrrolidine-2-carboxylate (7.60 g, 26.54 mmol) in THF (50.00 mL) were added H2O (50.00 mL) and LiOH (6.36 g, 265.43 mmol) in portions at rt under air atmosphere. The resulting mixture was stirred for 2 h at rt under air atmosphere. On completion, reaction mixture was concentrated under reduced pressure. The residue was purified by reverse phase flash chromatography (Column: WelFlash™ C18-I, 20-40 um, 120 g; Eluent A: Water (plus 0.1% FA); Eluent B: ACN; Gradient: 15%-55% B in 25 min; Flow rate: 80 mL/min; Detector: 220/254 nm; desired fractions were collected at 25% B) and concentrated under reduced pressure to the title compound (3.2 g, 44% yield) as a white solid. 1H NMR (300 MHz, DMSO-d6) δ 12.82 (s, 1H), 6.75-6.70 (m, 1H), 4.24-4.20 (m, 1H), 3.80-3.69 (m, 1H), 3.49-3.40 (m, 1H), 3.85-3.98 (m, 2H), 2.13-1.91 (m, 3H), 1.85-1.68 (m, 1H), 1.39 (s, 9H); LC/MS (ESI, m/z): [(M+H)]+=273.1.

### 2-[(1s,4s)-4-[[1-(2,6-dioxopiperidin-3-yl)-3-methyl-2-oxo-1,3-benzodiazol-5-yl]methyl]cyclohexyl]ethyl 4-methylbenzenesulfonate (Intermediate CB)

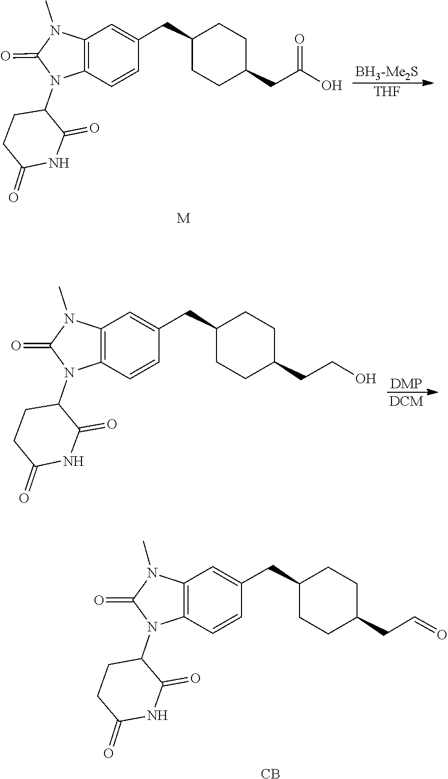

Step 1—3-(3-methyl-2-oxo-5-[[(1s,4s)-4-(2-hydroxyethyl)cyclohexyl]methyl]-1,3-benzodiazol-1-yl)piperidine-2,6-dione. To a stirred solution of [(1s,4s)-4-[[1-(2,6-dioxopiperidin-3-yl)-3-methyl-2-oxo-1,3-benzodiazol-5-yl]methyl]cyclohexyl]acetic acid (50.00 mg, 0.12 mmol, Intermediate M) in THF (2.00 mL) was added BH3-Me2S (36.75 mg, 0.484 mmol) dropwise at 0° C. under nitrogen atmosphere. The resulting mixture was stirred for 1 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH4HCO3); Eluent B: ACN; Gradient: 20%-50% B in 25 min; Flow rate: 60 mL/min; Detector: 220/254 nm; desired fractions were collected at 38% B) and concentrated under reduced pressure to afford the title compound (340 mg, 70% yield) as a white solid. 1H NMR (300 MHz, Chloroform-d) δ 6.94-6.67 (m, 4H), 3.73-3.69 (m, 3H), 3.43 (s, 3H), 2.95-2.75 (m, 2H), 2.63-2.59 (m, 3H), 2.29-2.18 (m, 1H), 1.76-1.72 (m, 3H), 1.63-1.59 (m, 2H), 1.50-1.36 (m, 8H); LC/MS (ESI, m/z): [(M+H)]+=400.3.

Step 2—2-[(1s,4s)-4-[[1-(2,6-dioxopiperidin-3-yl)-3-methyl-2-oxo-1,3-benzodiazol-5-yl]methyl]cyclohexyl]ethyl 4-methylbenzenesulfonate. To a stirred solution of 3-(3-methyl-2-oxo-5-[[(1s,4s)-4-(2-hydroxyethyl)cyclohexyl]methyl]-1,3-benzodiazol-1-yl)piperidine-2,6-dione (170.00 mg, 0.426 mmol) in DCM (5.00 mL) was added DMP (198.54 mg, 0.468 mmol) in portions at rt under nitrogen atmosphere. The resulting mixture was stirred for 1 h at rt under nitrogen atmosphere. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L NH4HCO3); Eluent B: ACN; Gradient: 35%-65% B in 25 min; Flow rate: 60 mL/min; Detector: 220/254 nm; desired fractions were collected at 48% B) and concentrated under reduced pressure to afford the title compound (80 mg, 47% yield) as a light brown solid. 1H NMR (300 MHz, Chloroform-d) δ 9.78-9.75 (m, 1H), 6.89-6.66 (m, 3H), 5.29-5.14 (m, 1H), 3.51-3.36 (m, 3H), 2.99-2.66 (m, 2H), 2.65-2.58 (m, 2H), 2.48-2.29 (m, 2H), 2.27-2.12 (m, 2H), 2.08-1.88 (m, 1H), 1.81-1.68 (m, 1H), 1.66-1.40 (m, 6H), 1.38-1.22 (m, 3H); LC/MS (ESI, m/z): [(M+H)]+=554.2.

### (4S)-4-Amino-5-(2-chloro-3-[4-[1-(2,6-dioxopiperidin-3-yl)-3-methyl-2-oxo-1,3-benzodiazol-5-yl]butyl]phenoxy)pentanamide (Intermediate CC)

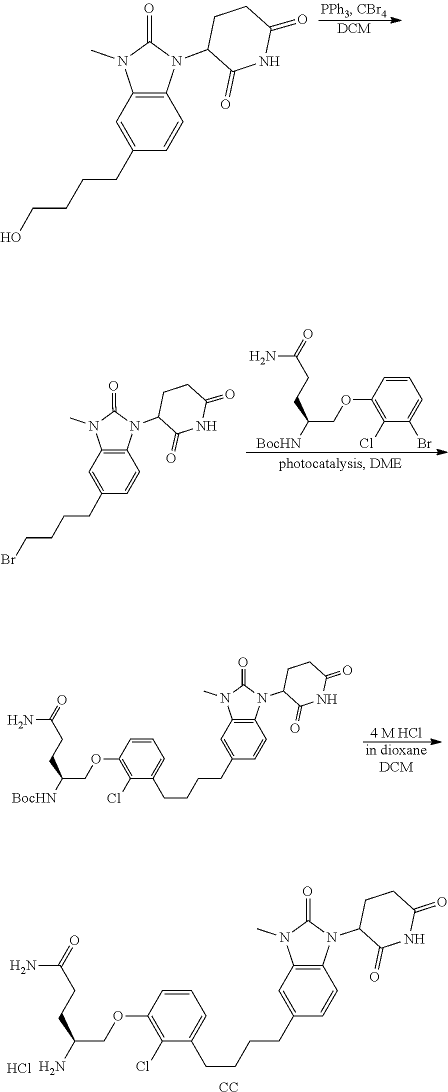

Step 1—3-[5-(4-bromobutyl)-3-Methyl-2-oxo-1,3-benzodiazol-1-yl]piperidine-2,6-dione. To a stirred solution of 3-[5-(4-hydroxybutyl)-3-methyl-2-oxo-1,3-benzodiazol-1-yl]piperidine-2,6-dione (1.99 g, 6.00 mmol, CAS #: 2408504-92-5) in DCM (20.00 mL) and THF (10.00 mL) were added CBr₄ (5.97 g, 18.02 mmol) and PPh₃ (3.23 g, 12.31 mmol) at 0° C. under nitrogen atmosphere. The resulting mixture was stirred for 2 h at rt under nitrogen atmosphere. On completion, the reaction mixture was concentrated under reduced pressure. The residue was purified by reverse flash chromatography (Column, Welflash™ C18-1,20-40 um, 330 g; Eluent A: Water (plus 10 mmol/L FA); Eluent B: ACN; Gradient 30% to 60% B in 30 min; Flow rate: 80 mL/min; Detector: 220/254 nm; desired fractions were collected at 37% B) and concentrated under reduced pressure to afford the title compound (1.0 g, 42% yield) as a white solid. ¹H NMR (300 MHz, Chloroform-d) δ 8.40 (s, 1H), 6.94-6.82 (m, 2H), 6.78-6.68 (m, 1H), 5.24 (dd, J=12.5, 5.3 Hz, 1H), 3.45 (s, 3H), 2.99-2.82 (m, 2H), 2.81-2.61 (m, 4H), 2.29-2.18 (m, 1H), 1.98-1.72 (m, 5H); LC/MS (ESI, m/z): [(M+H)]+=394.2, 396.2.

Step 2—Tert-butyl N-[(2S)-4-carbamoyl-1-(2-chloro-3-[4-[1-(2,6-dioxopiperidin-3-yl)-3-methyl-2-oxo-1,3-benzodiazol-5-yl]butyl]phenoxy)butan-2-yl]carbamate. To a 40 mL vial equipped with a stir bar was added photocatalyst IR[DF(CF₃)PPY]₂(DTBPY)PF₆ (56.92 mg, 0.051 mmol), 3-[5-(4-bromobutyl)-3-methyl-2-oxo-1,3-benzodiazol-1-yl]piperidine-2,6-dione (2.00 g, 5.07 mmol), tert-butyl N-[(2S)-1-(3-bromo-2-chlorophenoxy)-4-carbamoylbutan-2-yl]carbamate (3.21 g, 7.61 mmol, synthesized via Steps 1-3 of Intermediate C), tris(propan-2-yl)silane (803.29 mg, 5.07 mmol), and anhydrous sodium carbonate (1.61 g, 15.22 mmol). The vial was sealed and placed under nitrogen before 8 mL of DME was added. To a separate vial was added 1,2-dimethylhydrazine dihydrochloride (6.75 mg, 0.051 mmol) and 4,4′-di-tert-butyl-2,2′-bipyridine (13.61 mg, 0.051 mmol). The catalyst vial was sealed, purged with nitrogen then to it was added 6 mL of DME. The precatalyst solution was sonicated for 5 min, after which, 6 mL (0.5% mol catalyst, 2.5 mmol) was syringed into the reaction vessel. The solution was degassed by sparging with nitrogen while stirring for 10 minutes before sealing with Parafilm. The reaction was stirred and irradiated with a 34 W blue LED lamp (with cooling fan to keep the reaction temperature at 25° C.) for 16 hours. On completion, the reaction was quenched by exposure to air and concentrated in vacuo. The residue was purified by reverse flash chromatography (column, Welflash™ C18-1,20-40 um, 330 g; Eluent A: Water (plus 10 mmol/L FA); Eluent B: ACN; Gradient 40% to 70% B in 30 min; Flow rate: 80 mL/min; Detector: 220/254 nm; desired fractions were collected at 38% B) and concentrated under reduced pressure to afford the title compound (792 mg, 24% yield) as a white solid. ¹H NMR (300 MHz, Chloroform-d) δ 8.70-8.68 (m, 1H), 7.14-7.06 (m, 1H), 6.94-6.80 (m, 3H), 6.77-6.66 (m, 2H), 6.44-6.34 (m, 1H), 5.84-5.71 (m, 1H), 5.28-5.11 (m, 2H), 3.42 (s, 3H), 3.01-2.62 (m, 8H), 2.42-2.30 (m, 2H), 2.25-2.13 (m, 1H), 2.11-1.95 (m, 3H), 1.77-1.58 (m, 4H), 1.46 (s, 9H); LC/MS (ESI, m/z): [(M+H)]+=656.2.

Step 3—(4S)-4-Amino-5-(2-chloro-3-[4-[1-(2,6-dioxopiperidin-3-yl)-3-methyl-2-oxo-1,3-benzodiazol-5-yl]butyl]phenoxy)pentanamide hydrochloride. To a stirred solution of tert-butyl N-[(2S)-4-carbamoyl-1-(2-chloro-3-[4-[1-(2,6-dioxopiperidin-3-yl)-3-methyl-2-oxo-1,3-benzodiazol-5-yl]butyl]phenoxy)butan-2-yl]carbamate (790.00 mg, 1.204 mmol) in DCM (9.00 mL) was added a solution of 4 M HCl (gas) in 1,4-dioxane (4.00 mL) dropwise at rt under nitrogen atmosphere. The resulting mixture was stirred for 3 h at rt under nitrogen atmosphere. On completion, the reaction mixture was concentrated under reduced pressure to afford the title compound (730 mg, 99% crude yield) as a light green solid. ¹H NMR (300 MHz, DMSO-d6) δ11.06 (s, 1H), 8.42-8.29 (broad, 3H), 7.53-7.36 (m, 1H), 7.28-7.12 (m, 1H), 7.08-6.77 (m, 6H), 5.36-5.30 (m, 1H), 4.25-4.08 (m, 2H), 3.62-3.49 (m, 5H), 2.96-2.83 (m, 1H), 2.77-2.59 (m, 5H), 2.35-2.20 (m, 2H), 2.03-1.88 (m, 3H), 1.65-1.55 (m, 4H); LC/MS (ESI, m/z): [(M+H)]+=556.2.

### 3-(1-aminocyclopentyl)propenamide (Intermediate CD)

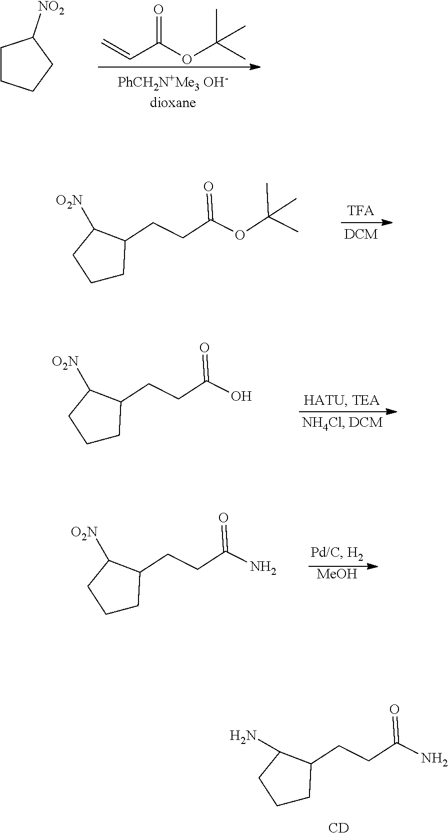

Step 1—Tert-butyl 3-(1-nitrocyclopentyl)propanoate. To a stirred solution of nitrocyclopentane (1.50 g, 13.39 mmol) and benzyltrimethylammonium hydroxide (163.42 mg, 0.391 mmol, 40% solution) in dioxane (15.00 mL) was added tert-butyl prop-2-enoate (1.67 g, 13.03 mmol) dropwise at 70° C. under nitrogen atmosphere. The resulting mixture was stirred for overnight at 70° C. under nitrogen atmosphere. On completion, the reaction was quenched with water (50 mL) at rt. The resulting mixture was then extracted with EtOAc (3×100 mL). The combined organic layers were washed with brine (3×50 mL), dried over anhydrous Na2SO4, filtered and concentrated under reduced pressure. The residue was purified by silica gel column chromatography, eluted with PE/EA=10/1, to afford the title compound (3 g, 95% yield) as a light brown oil. ¹H NMR (300 MHz, Chloroform-d) δ 2.63-2.47 (m, 2H), 2.32-2.16 (m, 4H), 1.85-1.67 (m, 6H), 1.44 (s, 9H).

Step 2—3-(1-nitrocyclopentyl)propanoic acid. To a stirred solution of tert-butyl 3-(1-nitrocyclopentyl)propanoate (3 g) in DCM (20 mL) was added TFA (5.00 mL, 67.31 mmol) dropwise at rt under air atmosphere. The resulting mixture was stirred for 2 h at rt under air atmosphere. On completion, the reaction mixture was concentrated under reduced pressure to give the title compound (2.3 g, 90% yield) as a light brown oil. ¹H NMR (300 MHz, Chloroform-d) δ 9.70 (s, 1H), 2.64-2.55 (m, 2H), 2.49-2.27 (m, 4H), 1.86-1.73 (m, 6H).

Step 3—3-(1-nitrocyclopentyl)propenamide. To a stirred solution/mixture of 3-(1-nitrocyclopentyl)propanoic acid (1.30 g, 6.95 mmol) and TEA (2.90 mL, 20.84 mmol) in DCM (20.00 mL) were added NH4Cl (0.74 g) and HATU (1.85 g, 4.86 mmol) in portions at rt under air atmosphere. The resulting mixture was stirred for 4 h at rt under nitrogen atmosphere. On completion, the reaction mixture was concentrated under reduced pressure. The crude product was purified by reverse phase flash chromatography (Column: Spherical C18 Column, 20-40 um, 120 g; Mobile Phase A: Water (0.1% NH₄HCO₃), Mobile Phase B: ACN; Flow rate: 60 mL/min; Gradient: 20% B to 50% B in 25 min, 254 nm; the fractions containing the desired product were collected at 38% B) to afford the title compound (0.95 g, 74% yield) as a light brown solid. ¹H NMR (300 MHz, Chloroform-d) δ 5.74-5.67 (m, 2H), 2.64-2.50 (m, 2H), 2.41-2.32 (m, 2H), 2.27-2.17 (m, 2H), 1.88-1.71 (m, 6H); LC/MS (ESI, m/z): [(M+1)]+=187.3.

Step 4—3-(1-aminocyclopentyl)propenamide. To a solution of 3-(1-nitrocyclopentyl)propanamide (950.00 mg, 5101 mmol) in MeOH (10 mL) was added Pd/C (542.93 mg, 0.510 mmol, 10 wt %) under argon or nitrogen atmosphere. The reaction system was degassed under vacuum and purged with H2 several times. Then the reaction mixture was hydrogenated under H2 balloon (˜1 atm) at 25° C. for 48 h. After completion of the reaction, Pd/C was filtered off through celite and the filter cake was washed with MeOH (3×30 mL). The corresponding filtrate was concentrated under reduced pressure to provide the title compound (800 mg, 90% yield) as alight brown solid. ¹H NMR (300 MHz, Chloroform-d) δ 6.21-5.80 (m, 1H), 5.58-5.43 (m, 1H), 2.45-2.31 (m, 3H), 2.07-2.02 (m, 1H), 1.88-1.79 (m, 1H), 1.77-1.61 (m, 8H), 1.58-1.40 (m, 1H); LC/MS (ESI, m/z): [(M+H)]+=157.3.

### Tert-butyl N-[(2S, 11S)-6-bromo-2-(dimethylcarbamoyl)-12-oxo-1-azatricyclo[6.4.1.0\hat{ }[4,] trideca-4(13), 5, 7-trien-11-yl] carbamate (Intermediate CE)

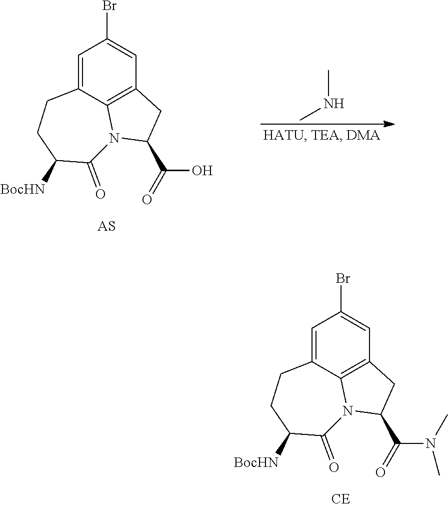

To a stirred solution of (2S,11S)-6-bromo-11-[(tert-butoxycarbonyl) amino]-12-oxo-1-azatricyclo[6.4.1.0[4,13]]trideca-4(13),5,7-triene-2-carboxylic acid (1.50 g, 3.53 mmol, Intermediate AS) and dimethylamine hydrochloride (0.32 g, 3.88 mmol) in DMA (20.00 mL) and TEA (1.07 g, 10.58 mmol) was added HATU (1.61 g, 4.23 mmol) at 0° C. under nitrogen atmosphere. The resulting mixture was stirred for 2 h at 25° C. under nitrogen atmosphere. On completion, the reaction mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-65% B in 30 min; Flow rate: 80 mL/min; Detector: 220/254 nm; desired fractions were collected at 53% B) and concentrated under reduced pressure to afford the title compound (1.03 g, 65% yield) as a white solid. ¹H NMR (300 MHz, Chloroform-d) δ 7.22-7.11 (m, 2H), 5.56-5.47 (m, 1H), 4.29-4.20 (m, 1H), 3.53-3.36 (m, 1H), 3.34-3.18 (m, 1H), 3.16 (s, 3H), 3.13-2.99 (m, 1H), 2.96 (s, 3H), 2.95-2.87 (m, 1H), 2.39-2.08 (m, 2H), 1.45 (s, 9H); LC/MS (ESI, m/z): [(M+H)]+=452.2, 454.2.

### (2S)-1-[(2S)-2-(6-[3-[(2S)-2-amino-4-carbamoylbutoxy]-2-fluoro-5-methylphenyl]hexanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate CF)

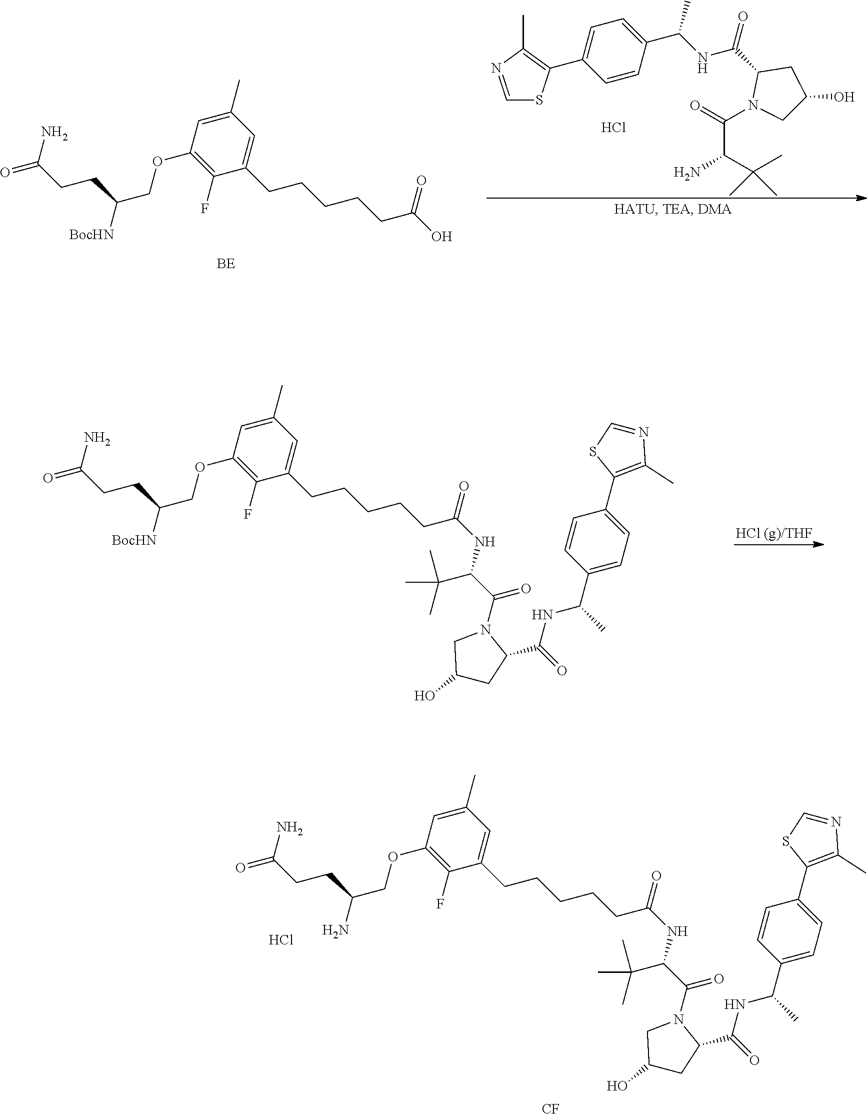

Step 1—Tert-butyl N-[(2S)-4-carbamoyl-1-[2-fluoro-3-(5-[[(2S)-1-[(2S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]pentyl)-5-methylphenoxy]butan-2-yl]carbamate. To a stirred mixture of 6-[3-[(2S)-2-[(tert-butoxycarbonyl)amino]-4-carbamoylbutoxy]-2-fluoro-5-methylphenyl]hexanoic acid (634.00 mg, 1.395 mmol, Intermediate BE) and (2S,4S)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (805.17 mg, 1.674 mmol) in DMA (8.00 mL) were added TEA (705.71 mg, 6.974 mmol) and HATU (636.42 mg, 1.674 mmol) in turns at rt under nitrogen atmosphere. The resulting mixture was stirred for 1 h at rt under nitrogen atmosphere. On completion, the reaction mixture was purified by reverse flash chromatography (Dolumn, C18 silica gel; mobile phase, MeCN in water (10 mmol/L NH₄HCO₃), 35%-60% gradient in 25 min; detector, UV 210/254 nm) to afford the title compound (1.03 g, 84% yield) as a white solid. ¹H NMR (400 MHz, Chloroform-d) δ 8.74 (s, 1H), 7.75 (d, J=7.9 Hz, 1H), 7.47-7.38 (m, 4H), 6.64-6.55 (m, 2H), 6.36 (s, 1H), 6.20 (d, J=9.1 Hz, 1H), 5.70 (s, 1H), 5.51-5.40 (m, 1H), 5.16-5.04 (m, 2H), 4.75 (d, J=8.9 Hz, 1H), 4.61 (d, J=9.1 Hz, 1H), 4.50-4.45 (m, 1H), 4.06-4.01 (m, 2H), 4.01-3.92 (m, 2H), 3.89-3.82 (m, 1H), 2.66-2.58 (m, 2H), 2.57 (s, 3H), 2.41-2.31 (m, 3H), 2.29-2.21 (m, 5H), 2.20-2.12 (m, 1H), 2.06-1.99 (m, 2H), 1.74-1.58 (m, 4H), 1.52 (d, J=6.9 Hz, 3H), 1.47 (s, 9H), 1.41-1.32 (m, 2H), 1.07 (s, 9H); LC/MS (ESI, m/z): [(M+H)]+=881.5.

Step 2—(2S)-1-[(2S)-2-(6-[3-[(2S)-2-amino-4-carbamoylbutoxy]-2-fluoro-5-methylphenyl]hexanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred mixture of tert-butyl N-[(2S)-4-carbamoyl-1-[2-fluoro-3-(5-[[(2S)-1-[(2S)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]pentyl)-5-methylphenoxy]butan-2-yl]carbamate (1.03 g, 1.169 mmol) in THF (6.00 mL) was added HCl (gas) in 1,4-dioxane (6.00 mL, 105.105 mmol) at rt under nitrogen atmosphere. The resulting mixture was stirred for 1 h at rt under nitrogen atmosphere. On completion, the reaction mixture was concentrated under vacuum to afford the title compound (1.25 g, crude) as a white solid. ¹H NMR (400 MHz, DMSO-d6) δ 9.06 (s, 1H), 8.41-8.29 (m, 4H), 7.81 (d, J=8.8 Hz, 1H), 7.47-7.43 (m, 2H), 7.42-7.38 (m, 2H), 6.94 (s, 1H), 6.90-6.83 (m, 1H), 6.71-6.65 (m, 1H), 4.98-4.87 (m, 2H), 4.44 (d, J=8.8 Hz, 1H), 4.37-4.29 (m, 1H), 4.24-4.14 (m, 2H), 4.13-4.06 (m, 1H), 3.94-3.86 (m, 1H), 3.53-3.49 (m, 1H), 2.56-2.52 (m, 2H), 2.47 (s, 3H), 2.36-2.27 (m, 3H), 2.25 (s, 3H), 2.24-2.19 (m, 1H), 2.16-2.05 (m, 1H), 1.96-1.85 (m, 2H), 1.69-1.61 (m, 1H), 1.59-1.42 (m, 5H), 1.38 (d, J=7.0 Hz, 3H), 1.32-1.21 (m, 2H), 0.95 (s, 9H). LC/MS (ESI, m/z): [(M+H)]+=791.5.

### (4R)-4-amino-N-methylpentanamide (Intermediate CG)

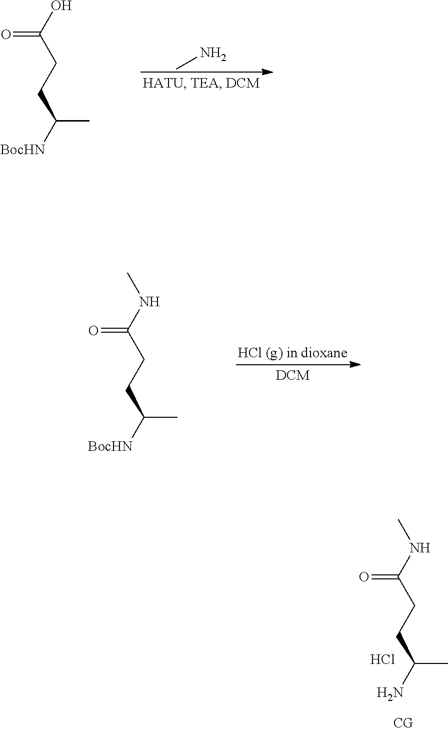

Step 1—Tert-butyl N-[(2R)-4-(methylcarbamoyl)butan-2-yl]carbamate. To a stirred solution of (4R)-4-[(tert-butoxycarbonyl)amino]pentanoic acid (3.00 g, 13.81 mmol, CAS #214402-34-3) in DCM (30.00 mL) were added TEA (9.60 mL, 69.08 mmol), HATU (6.83 g, 17.95 mmol) and 2 M methylamine in THF (13.8 mL, 27.69 mmol) in portions at rt under nitrogen atmosphere. The resulting mixture was stirred for 2 h at rt under nitrogen atmosphere. On completion, the residue was purified by reverse phase flash chromatography (Column: WelFlash™ C18-I, 20-40 ?m, 330 g; Eluent A: Water (plus 10 mmol/L NH4HCO3); Eluent B: ACN; Gradient: 20%-60% B in 25 min; Flow rate: 85 mL/min; Detector: 220/254 nm; desired fractions were collected at 50% B) and concentrated under reduced pressure to afford the title compound (2 g, 63% yield) as a white solid. ¹H NMR (400 MHz, Chloroform-d) δ 6.62-6.75 (m, 2H), 2.78 (s, 3H), 2.28-2.23 (m, 1H), 2.18-2.11 (m, 2H), 1.77-1.74 (m, 1H), 1.65-1.61 (m, 1H), 1.42 (s, 9H), 1.15-1.07 (m, 3H); LC/MS (ESI, m/z): [(M+H)]+=231.2.

Step 2—(4R)-4-amino-N-methylpentanamide hydrochloride. To a stirred solution of tert-butyl N-[(2R)-4-(methylcarbamoyl)butan-2-yl]carbamate (2.00 g, 8.68 mmol) in dioxane (20.00 mL) was added HCl (gas) in 1,4-dioxane (10.00 mL) at 0° C. under nitrogen atmosphere. The resulting mixture was stirred for 2 h at 25° C. under nitrogen atmosphere. On completion, the reaction mixture was concentrated under reduced pressure to afford the title compound (1.3 g, 90% yield) as a light yellow solid. 1H NMR (400 MHz, Chloroform-d) δ 6.61-6.51 (m, 2H), 2.85 (s, 3H), 2.48-2.40 (m, 1H), 2.18-2.11 (m, 2H), 1.79-1.73 (m, 1H), 1.64-1.58 (m, 1H), 1.21-1.12 (m, 3H); LC/MS (ESI, m/z): [(M+H)]+=131.3.

### Tert-butyl N-[(2S,11S)-6-bromo-2-[[(2R)-4-(methylcarbamoyl)butan-2-yl]carbamoyl]-12-oxo-1-azatricyclo[6.4.1.0\hat{ }[4,13]]trideca-4(13),5,7-trien-11-yl]carbamate (Intermediate CH)

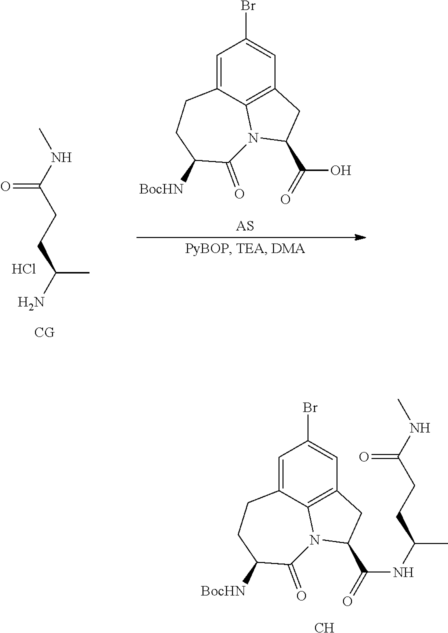

To a stirred solution of (4R)-4-amino-N-methylpentanamide hydrochloride (0.60 g, 3.60 mmol, Intermediate CG) and (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (1.84 g, 4.33 mmol, Intermediate AS) in DMA (15.00 mL) were added TEA (2.5 mL, 17.99 mmol) and PyBOP (2.44 g, 4.68 mmol) at 0° C. under nitrogen atmosphere. The resulting mixture was stirred for 16 h at 25° C. under nitrogen atmosphere. On completion, the reaction mixture was directly purified by reverse phase flash chromatography (Column: WelFlash™ C18-I, 20-40 um, 330 g; EluentA: Water (10 mmol/L NH4HCO3); Eluent B: ACN; Gradient: 10%-60% B in 30 min; Flow rate: 80 mL/min; Detector: 220/254 nm; desired fractions were collected at 55% B) and concentrated under reduced pressure to afford the title compound (1 g, 52% yield) as a yellow solid. ¹H NMR (400 MHz, Chloroform-d) δ 7.27-7.24 (m, 1H), 7.22-7.18 (m, 1H), 6.83-6.73 (m, 1H), 6.25-6.18 (m, 1H), 5.85-5.81 (m, 1H), 5.21-5.15 (m, 1H), 4.28-4.21 (m, 1H), 3.51-3.44 (m, 1H), 3.29-3.24 (m, 1H), 3.08-3.05 (m, 1H), 2.74 (s, 3H), 2.42-2.38 (m, 1H), 2.10-2.05 (m, 3H), 2.03-1.98 (m, 2H), 1.89-1.62 (m, 2H), 1.48 (s, 9H), 1.25-1.18 (m, 3H); LC/MS (ESI, m/z): [(M+H)]+=537.2; 539.2.

### (R)-4-amino-N,N-dimethylpentanamide (Intermediate CI)

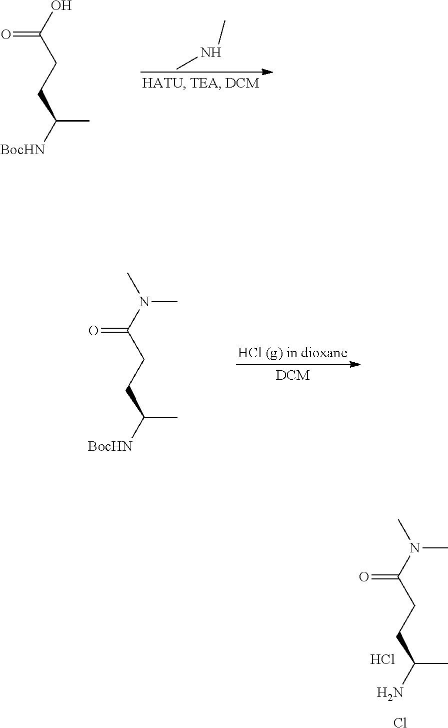

Step 1—Tert-butyl N-[(2R)-4-(dimethylcarbamoyl)butan-2-yl]carbamate. To a stirred solution of (4R)-4-[(tert-butoxycarbonyl)amino]pentanoic acid (3.00 g, 13.81 mmol) in DCM (30.00 mL) were added TEA (9.6 mL, 69.04 mmol) and HATU (6.83 g, 17.95 mmol) and 2 M dimethylamine in THF (13.87 mL, 7.75 mmol) in portions at rt under nitrogen atmosphere. The resulting mixture was stirred for 2 h at rt under nitrogen atmosphere. On completion, the reaction mixture was concentrated under vacuum. Then the residue was purified by reverse phase flash chromatography with the following conditions: Column: WelFlash™ C18-I, 330 g; Eluent A: Water (plus 10 mmol/L NH₄HCO₃); Eluent B: ACN; Gradient: 20%-60% B in 25 min; Flow rate: 85 mL/min; Detector: 220/254 nm; desired fractions were collected at 55% B) and concentrated under reduced pressure to afford the title compound (2 g, 59% yield) as a white solid. ¹H NMR (400 MHz, Chloroform-d) δ 7.75-7.70 (m, 1H), 3.73-3.68 (m, 1H), 2.78 (s, 6H), 2.26-2.15 (m, 2H), 1.83-1.60 (m, 2H), 1.42 (s, 9H), 1.15-0.98 (m, 3H); LC/MS (ESI, m/z): [(M+H)]+=245.3.

Step 2—(R)-4-amino-N,N-dimethylpentanamide hydrochloride. To a stirred solution of tert-butyl N-[(2R)-4-(dimethylcarbamoyl)butan-2-yl]carbamate (2.00 g, 8.19 mmol) in dioxane (20.00 mL) was added HCl (gas) in 1,4-dioxane (10.00 mL) at 0° C. under nitrogen atmosphere. The resulting mixture was stirred for 2 h at 25° C. under nitrogen atmosphere. On completion, the reaction mixture was concentrated under reduced pressure to afford the title compound (1.2 g, 81% yield) as a light yellow solid. ¹H NMR (400 MHz, Chloroform-d) δ 8.38 (s, 2H), 3.57 (s, 6H), 3.34-3.22 (m, 1H), 2.93-2.88 (m, 2H), 2.08-1.80 (m, 2H), 1.36-1.28 (m, 3H); LC/MS (ESI, m/z): [(M+H)]+=145.3.

### Tert-butyl N-[(2S,11S)-6-bromo-2-[[(2R)-4-(dimethylcarbamoyl)butan-2-yl]carbamoyl]-12-oxo-1-azatricyclo[6.4.1.0\hat{ }[4,13]]trideca-4(13),5,7-trien-11-yl]carbamate (Intermediate CJ)

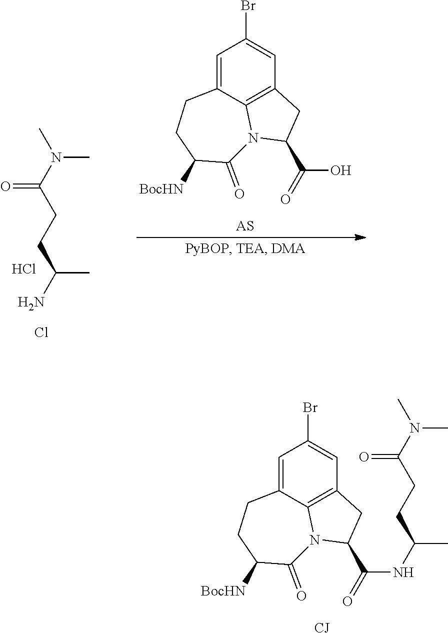

To a stirred solution of (4R)-4-amino-N,N-dimethylpentanamide hydrochloride (600.00 mg, 3.32 mmol, Intermediate CI) and (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (1694.71 mg, 3.99 mmol, Intermediate AS) in DMA (15.00 mL) were added TEA (1680.15 mg, 16.60 mmol) and PyBOP (2246.55 mg, 4.32 mmol) at 0° C. under nitrogen atmosphere. The resulting mixture was stirred for 16 h at 25° C. under nitrogen atmosphere. On completion, the mixture was purified directly by reverse phase flash chromatography (Column: WelFlash™ C18-I, 20-40 um, 330 g; Eluent A: Water (10 mmol/L NH4HCO3); Eluent B: ACN; Gradient: 15%-60% B in 30 min; Flow rate: 80 mL/min; Detector: 220/254 nm; desired fractions were collected at 57% B) and concentrated under reduced pressure to afford the title compound (1 g, 55% yield) as a yellow solid. ¹H NMR (400 MHz, Chloroform-d) δ 7.27-7.21 (m, 2H), 7.20-7.11 (m, 1H), 7.03-6.95 (m, 1H), 5.86-5.81 (m, 1H), 5.16-5.11 (m, 1H), 4.31-4.23 (m, 1H), 3.13-3.02 (m, 1H), 2.88 (s, 6H), 2.45-2.29 (m, 2H), 2.19-2.15 (m, 2H), 1.82-1.67 (m, 5H), 1.48 (s, 9H), 1.25-1.18 (m, 3H); LC/MS (ESI, m/z): [(M+H)]+=551.5; 553.5.

### tert-butyl ((3S,6S)-9-bromo-6-(methylcarbamoyl)-4-oxo-1,2,3,4,6,7-hexahydroazepino[3,2,1]indol-3-yl)carbamate (Intermediate CK)

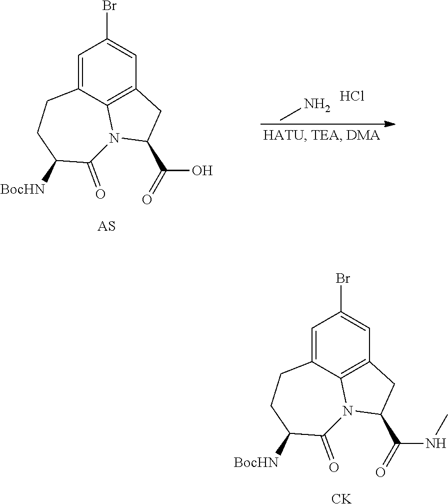

To a stirred solution of (2S,11S)-6-bromo-11-[(tert-butoxycarbonyl)amino]-12-oxo-1-azatricyclo[6.4.1.0\hat{ }{4,13}]trideca-4(13),5,7-triene-2-carboxylic acid (336 mg, 0.790 mmol, Intermediate AS) and methanamine hydrochloride (60.83 mg, 0.908 mmol) in DMA (4.00 mL) were added TEA (40.11 mg, 0.40 mmol) and HATU (60.28 mg, 0.16 mmol) at 0° C. under nitrogen atmosphere. The resulting mixture was stirred for 1 h at rt under nitrogen atmosphere. On completion, the reaction mixture was concentrated under reduced pressure. The residue was purified by reverse flash chromatography (Column, Welflash™ C18-1, 20-40 um, 120 g; Eluent A: Water (plus 10 mmol/L FA); Eluent B: ACN; Gradient 25% to 50% B in 30 min; Flow rate: 60 mL/min; Detector: 220/254 nm; desired fractions were collected at 39% B) and concentrated under reduced pressure to afford the title compound (200 mg, 47% yield) as a white solid. 1H NMR (300 MHz, Chloroform-d) δ 7.27 (s, 1H), 7.18 (s, 1H), 6.80-6.70 (m, 1H), 5.78-5.70 (m, 1H), 5.24-5.16 (m, 1H), 4.31-4.18 (m, 1H), 3.69-3.58 (m, 1H), 3.24-3.11 (m, 2H), 3.10-2.97 (m, 1H), 2.80 (s, 3H), 2.48-2.39 (m, 1H), 2.09-1.97 (m, 1H), 1.47 (s, 9H); LC/MS (ESI, m/z): [(M−H)]−=436.2, 438.2.

### 7-[1-(2,6-dioxopiperidin-3-yl)-3-methyl-2-oxo-1,3-benzodiazol-5-yl]heptanoic acid (Intermediate CL)

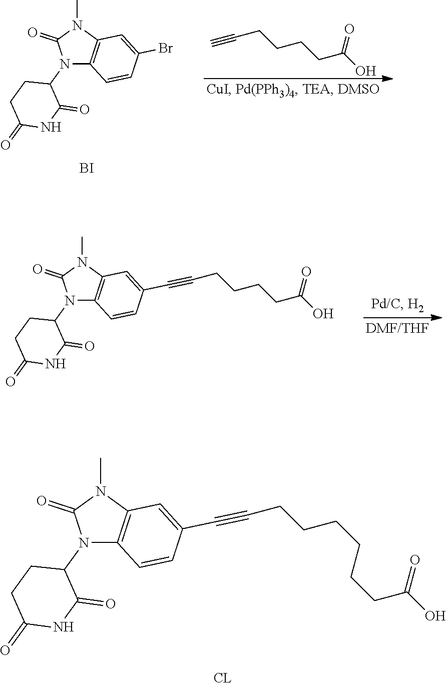

Step 1-7-[1-(2,6-dioxopiperidin-3-yl)-3-methyl-2-oxo-1,3-benzodiazol-5-yl]hept-6-ynoic acid. To a stirred solution of 3-(5-bromo-3-methyl-2-oxo-1,3-benzodiazol-1-yl)piperidine-2,6-dione (20.00 g, 59.143 mmol, Intermediate BI) in DMSO (200.00 mL) were added 6-heptynoic acid (14.92 g, 118.3 mmol) and TEA (50.00 mL) dropwise at rt under N₂ atmosphere. To the above mixture were added CuI (1.13 g, 5.91 mmol) and Pd(PPh₃)₄ (6.83 g, 5.9 mmol) in portions at rt. Next, the reaction mixture was stirred for 2 h at 80° C. under nitrogen atmosphere. The mixture was allowed to cool down to rt. On completion, the reaction mixture was concentrated under reduced pressure. The crude product was purified by reverse phase flash (Column: Spherical C18 Column, 20-40 um, 120 g; Mobile Phase A: Water (10 mmol/L NH₄HCO₃), Mobile Phase B: ACN; Flow rate: 60 mL/min; Gradient: 30% B to 50% B in 25 min, 254 nm. The fractions containing the desired product were collected at 42% B) to afford the title compound (14 g, 62% yield) as a white solid. ¹H NMR (400 MHz, DMSO-d6) δ12.05 (broad, 1H), 11.11 (s, 1H), 7.24 (d, J=1.2 Hz, 1H), 7.15-7.04 (m, 2H), 5.38 (dd, J=12.8, 5.4 Hz, 1H), 3.35 (s, 3H), 2.94-2.79 (m, 1H), 2.78-2.58 (m, 2H), 2.43 (t, J=6.9 Hz, 2H), 2.32-2.26 (m, 2H), 2.06-2.00 (m, 1H), 1.73-1.62 (m, 2H), 1.62-1.47 (m, 2H); LC/MS (ESI, m/z): [(M+H)]+=384.2.

Step 2-7-[1-(2,6-dioxopiperidin-3-yl)-3-methyl-2-oxo-1,3-benzodiazol-5-yl]heptanoic acid. To a stirred solution of 7-[1-(2,6-dioxopiperidin-3-yl)-3-methyl-2-oxo-1,3-benzodiazol-5-yl]hept-6-ynoic acid (14.00 g, 36.52 mmol) in DMF (100.00 mL) was added THF (200.00 mL) and Pd/C (0.39 g, 3.65 mmol) in portions at rt under nitrogen atmosphere. The resulting mixture was stirred for 16 h at 50° C. under hydrogen atmosphere. On completion, the mixture was cooled to rt. On completion, the reaction mixture was filtered, and the filter cake was washed with THF (3×20 mL). The filtrate was concentrated under reduced pressure. The crude product was purified by reverse phase flash chromatography (Column: Spherical C18 Column, 20-40 um, 120 g; Mobile Phase A: Water (0.1% NH4HCO3), Mobile Phase B: ACN; Flow rate: 60 mL/min; Gradient: 35% B to 55% B in 25 min, 254 nm; the fractions containing the desired product were collected at 42% B) to afford the title compound (7.8 g, 55% yield) as a yellow solid. 1H NMR (400 MHz, DMSO-d6) δ 11.92 (s, 1H), 11.08 (s, 1H), 7.03 (d, J=1.5 Hz, 1H), 7.00 (d, J=8.0 Hz, 1H), 6.86 (dd, J=8.1, 1.6 Hz, 1H), 5.34 (dd, J=12.8, 5.3 Hz, 1H), 3.34 (s, 3H), 2.92-2.87 (m, 1H), 2.80-2.66 (m, 1H), 2.66-2.57 (m, 3H), 2.20 (t, J=7.3 Hz, 2H), 2.06-1.96 (m, 1H), 1.66-1.55 (m, 2H), 1.50 (t, J=7.0 Hz, 2H), 1.35-1.27 (m, 4H); LC/MS (ESI, m/z): [(M+H)]+=388.2.

### (2S)-2-[[(5S,8S,10aR)-5-[(tert-butoxycarbonyl)amino]-3-[7-[1-(2,6-dioxopiperidin-3-yl)-3-methyl-2-oxo-1,3-benzodiazol-5-yl]heptanoyl]-6-oxo-octahydropyrrolo[1,2-a][1,5]diazocin-8-yl]formamido]-4-carbamoylbutanoic acid (Intermediate CM)

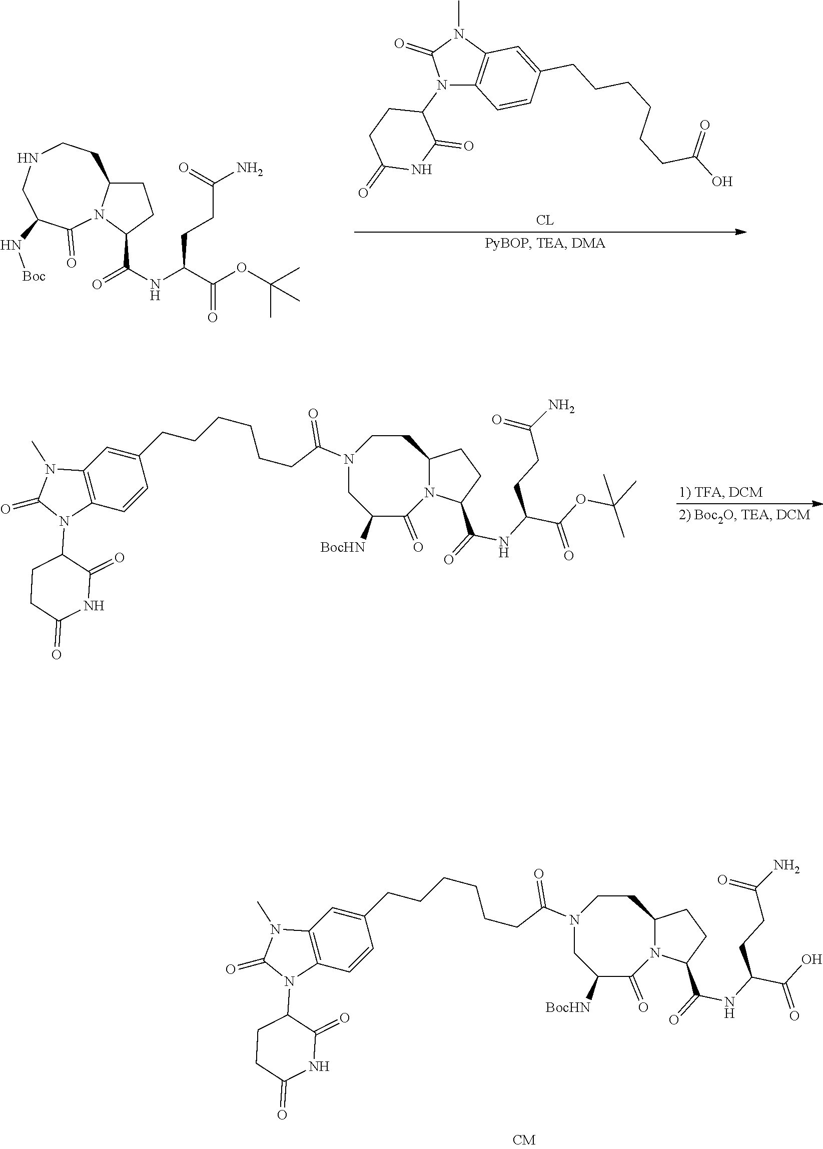 [SMILES: CC(C)(C)OC(=O)N[C@H]1CNCC[C@H]2CC[C@@H](C(=O)N[C@@H](CCC(N)=O)C(=O)OC(C)(C)C)N2C1=O ; Cn1c(=O)n(C2CCC(=O)NC2=O)c2ccc(CCCCCCC(=O)O)cc21 ; Cn1c(=O)n(C2CCC(=O)NC2=O)c2ccc(CCCCCCC(=O)N3CC[C@H]4CC[C@@H](C(=O)N[C@@H](CCC(N)=O)C(=O)OC(C)(C)C)N4C(=O)[C@@H](NC(=O)OC(C)(C)C)C3)cc21 ; Cn1c(=O)n(C2CCC(=O)NC2=O)c2ccc(CCCCCCC(=O)N3CC[C@H]4CC[C@@H](C(=O)N[C@@H](CCC(N)=O)C(=O)O)N4C(=O)[C@@H](NC(=O)OC(C)(C)C)C3)cc21]

Step 1—Tert-butyl (2S)-2-[[(5S,8S,10aR)-5-[(tert-butoxycarbonyl)amino]-3-[7-[1-(2,6-dioxopiperidin-3-yl)-3-methyl-2-oxo-1,3-benzodiazol-5-yl]heptanoyl]-6-oxo-octahydropyrrolo[1,2-a][1,5]diazocin-8-yl]formamido]-4-carbamoylbutanoate. To a solution of 7-[1-(2,6-dioxopiperidin-3-yl)-3-methyl-2-oxo-1,3-benzodiazol-5-yl]heptanoic acid (227.18 mg, 0.59 mmol, Intermediate CL), PyBOP (457.72 mg, 0.88 mmol) in DMA (6.00 mL) were added TEA (0.244 mL, 1.76 mmol) and tert-butyl (2S)-2-[[(5S,8S,10aR)-5-[(tert-butoxycarbonyl)amino]-6-oxo-octahydro-1H-pyrrolo[1,2-a][1,5]diazocin-8-yl]formamido]-4-carbamoylbutanoate (300.00 mg, 0.59 mmol, synthesized via Steps 1-2 of Intermediate AP) was added at 25° C. The resulting mixture was stirred for 1 h at rt under nitrogen atmosphere. On completion, the reaction mixture was purified directly by reverse phase flash chromatography (Column: WelFlash™ C18-I, 330 g; Eluent A: Water (plus 10 mmol/L HOAc); Eluent B: ACN; Gradient: 40%-60% B in 20 min; Flow rate: 80 mL/min; Detector: 220/254 nm; desired fractions were collected at 58% B) and concentrated under reduced pressure to afford the title compound (420 mg, 81% yield) as a light yellow solid. ¹H NMR (400 MHz, DMSO-d6) δ 11.09 (s, 1H), 8.31-8.25 (m, 1H), 7.23-7.18 (m, 1H), 7.05-6.94 (m, 2H), 6.89-6.93 (m, 1H), 6.78 (s, 1H), 6.56 (d, J=7.0 Hz, 1H), 5.34 (dd, J=12.7, 5.4 Hz, 1H), 4.50-4.34 (m, 2H), 4.19-4.07 (m, 2H), 3.73-3.58 (m, 2H), 3.38-3.33 (m, 2H), 3.32 (s, 3H), 3.27-3.06 (m, 2H), 2.97-2.83 (m, 1H), 2.78-2.56 (m, 4H), 2.44-2.37 (m, 1H), 2.18-2.15 (m, 3H), 1.97-1.86 (m, 2H), 1.85-1.71 (m, 3H), 1.68-1.46 (m, 6H), 1.40-1.31 (m, 18H), 1.35-1.30 (m, 4H); LC/MS (ESI, m/z): [(M+H)]+=881.7.

Step 2—(2S)-2-[[(5S,8S,10aR)-5-[(tert-butoxycarbonyl)amino]-3-[7-[1-(2,6-dioxopiperidin-3-yl)-3-methyl-2-oxo-1,3-benzodiazol-5-yl]heptanoyl]-6-oxo-octahydropyrrolo[1,2-a][1,5]diazocin-8-yl]formamido]-4-carbamoylbutanoic acid. To a stirred solution of tert-butyl (2S)-2-[[(5S,8S,10aR)-5-[(tert-butoxycarbonyl)amino]-3-[7-[1-(2,6-dioxopiperidin-3-yl)-3-methyl-2-oxo-1,3-benzodiazol-5-yl]heptanoyl]-6-oxo-octahydropyrrolo[1,2-a][1,5]diazocin-8-yl]formamido]-4-carbamoylbutanoate (420.00 mg, 0.48 mmol) in DCM (9.00 mL) was added TFA (3.00 mL) dropwise at rt under nitrogen atmosphere. The resulting mixture was stirred at rt for 4 h under nitrogen atmosphere. On completion, the reaction mixture was concentrated under reduced pressure. Next, the residue was re-dissolved in DCM (7.00 mL). Then to the mixture TEA (0.321 mL, 2.31 mmol) was added dropwise and Boc₂O (120.95 mg, 0.55 mmol) was added at 0° C. The resulting mixture was stirred overnight at rt under nitrogen atmosphere. On completion, the reaction mixture was concentrated under reduced pressure. The residue was purified by reverse phase flash chromatography (Column: WelFlash™ C18-I, 330 g; EluentA: Water (plus 10 mmol/L FA); Eluent B: ACN; Gradient: 35%-55% B in 15 min; Flow rate: 80 mL/min; Detector: 220/254 nm; desired fractions were collected at 47% B) and concentrated under reduced pressure to afford the title compound (300 mg, 79% yield) as a white solid. ¹H NMR (400 MHz, DMSO-d6) δ 12.42 (broad, 1H), 11.09 (s, 1H), 8.29-8.24 (m, 1H), 7.23-7.20 (m, 1H), 7.05-6.95 (m, 2H), 6.90-6.83 (m, 1H), 6.77 (s, 1H), 6.57 (d, J=6.9 Hz, 1H), 5.34 (dd, J=12.7, 5.3 Hz, 1H), 4.49-4.34 (m, 2H), 4.22-4.09 (m, 2H), 3.75-3.60 (m, 2H), 3.33 (s, 3H), 3.28-3.19 (m, 1H), 3.18-3.04 (m, 1H), 2.97-2.84 (m, 1H), 2.77-2.56 (m, 4H), 2.47-2.35 (m, 2H), 2.25-2.10 (m, 3H), 2.05-1.91 (m, 3H), 1.88-1.72 (m, 3H), 1.63-1.48 (m, 6H), 1.38 (s, 9H), 1.35-1.29 (m, 4H); LC/MS (ESI, m/z): [(M+H)]+=825.5.

### 1-(4-methanesulfonylphenyl)methanamine (Intermediate CN) (CAS #4393-16-2)

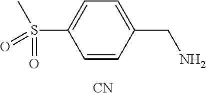

### [(1r,4r)-4-[[1-(2,6-dioxopiperidin-3-yl)-3-methyl-2-oxo-1,3-benzodiazol-5-yl]methyl]cyclohexyl]acetic acid (Intermediate CO)

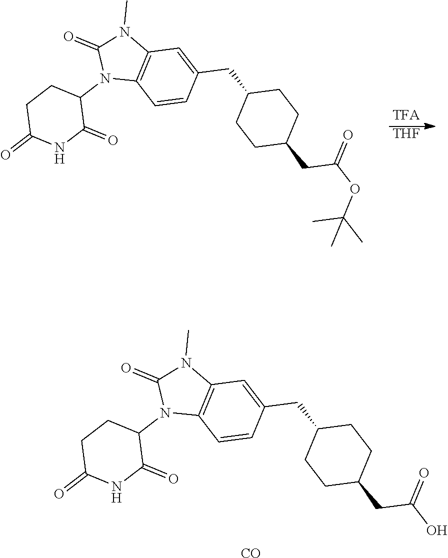 [SMILES: Cn1c(=O)n(C2CCC(=O)NC2=O)c2ccc(C[C@H]3CC[C@H](CC(=O)OC(C)(C)C)CC3)cc21 ; Cn1c(=O)n(C2CCC(=O)NC2=O)c2ccc(C[C@H]3CC[C@H](CC(=O)O)CC3)cc21 ; "THF" ; "CO"]

To a stirred solution of tert-butyl 2-[(1r,4r)-4-[[1-(2,6-dioxopiperidin-3-yl)-3-methyl-2-oxo-1,3-benzodiazol-5-yl]methyl]cyclohexyl]acetate (300.00 mg, 0.639 mmol, synthesized via Steps 1-6 of Intermediate M) in DCM (4.00 mL) was added TFA (2.00 m) at rt under nitrogen atmosphere. The resulting mixture was stirred for 1 h at rt under nitrogen atmosphere. On completion, the reaction mixture was concentrated under reduced pressure. The residue was washed with Et₂O (7 mL). The precipitated solids were collected by filtration and washed with Et₂O (3×3 mL) to afford the title compound (267 mg) as a white solid. ¹H NMR (400 MHz, DMSO-d6) δ 11.94 (s, 1H), 11.09 (s, 1H), 7.04-6.95 (m, 2H), 6.84-6.81 (m, 1H), 5.34 (dd, J=12.7, 5.3 Hz, 1H), 3.32 (s, 3H), 3.01-2.81 (m, 1H), 2.77-2.54 (m, 2H), 2.50-2.47 (m, 2H), 2.15-2.05 (m, 2H), 2.02-1.95 (m, 1H), 1.77-1.36 (m, 6H), 1.02-0.77 (m, 4H). LC/MS (ESI, m/z): [(M+H)]+=414.2.

### (2S)-2-[[(5S,8S,10aR)-5-[(tert-butoxycarbonyl)amino]-6-oxo-3-[2-[(1r,4r)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][15]diazocin-8-yl]formamido]-4-carbamoylbutanoic acid (Intermediate CP)

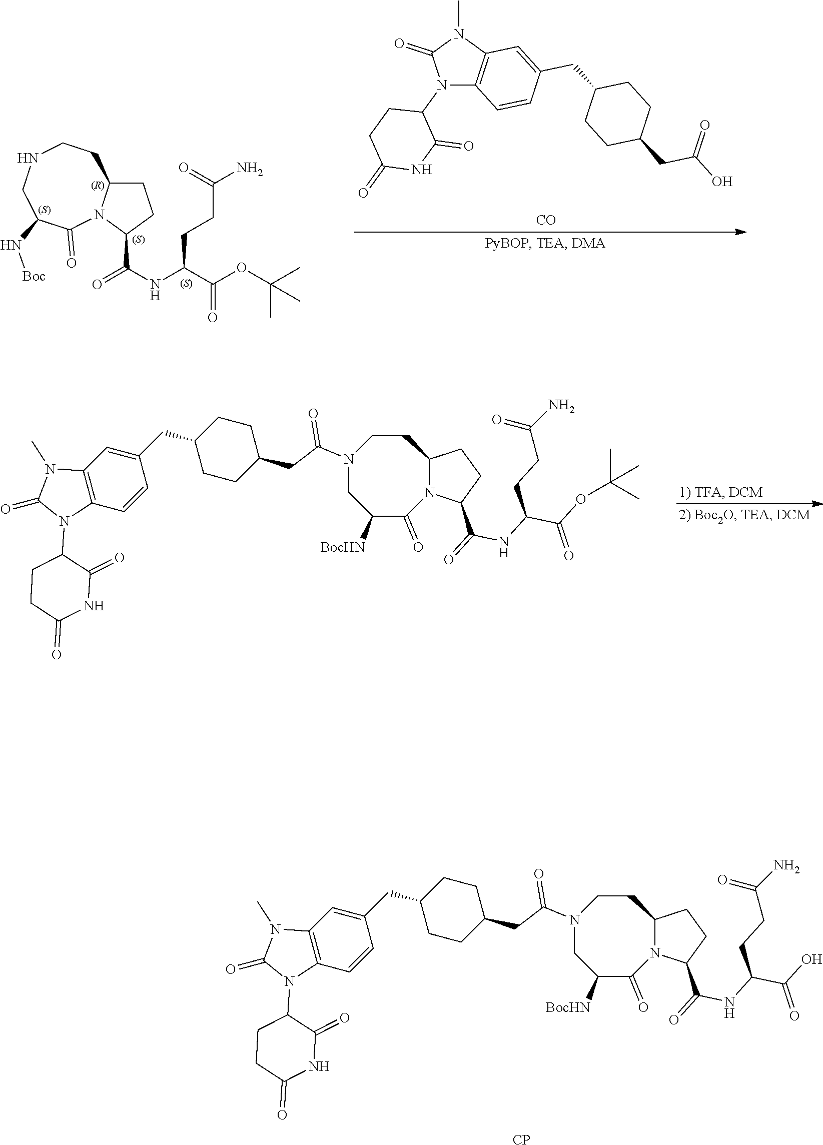

Step 1—Tert-butyl (2S)-2-[[(5S,8S,10aR)-5-[(tert-butoxycarbonyl)amino]-6-oxo-3-[2-[(1r,4r)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoate. To a stirred mixture of tert-butyl (2S)-2-[[(5S,8S,10aR)-5-[(tert-butoxycarbonyl)amino]-6-oxo-octahydro-1H-pyrrolo[1,2-a][1,5]diazocin-8-yl]formamido]-4-carbamoylbutanoate (300.00 mg, 0.586 mmol, synthesized via Steps 1-2 of Intermediate AP) and [(1R,4R)-4-[[1-(2,6-dioxopiperidin-3-yl)-3-methyl-2-oxo-1,3-benzodiazol-5-yl]methyl]cyclohexyl]acetic acid (266.69 mg, 0.645 mmol, Intermediate CO) in DMA (7.00 mL) were added TEA (0.408 mL, 2.93 mmol) and PyBOP (396.69 mg, 0.762 mmol) at rt under nitrogen atmosphere. The resulting mixture was stirred for 1 h at rt under nitrogen atmosphere. On completion, the reaction mixture was purified directly by reverse flash chromatography (Column: C18 silica gel; mobile phase, MeCN in water (10 mmol/L FA), 35% to 55% gradient in 25 min; detector, UV 220 nm) to afford the title compound (370 mg, 70% yield) as a white solid. ¹H NMR (400 MHz, DMSO-d6) δ 11.09 (s, 1H), 8.30-8.24 (m, 1H), 7.23-7.19 (m, 1H), 7.03-6.93 (m, 2H), 6.85-6.75 (m, 2H), 6.58 (d, J=7.1 Hz, 1H), 5.34 (dd, J=12.7, 5.4 Hz, 1H), 4.44-4.32 (m, 2H), 4.16-4.05 (m, 2H), 3.68-3.64 (m, 1H), 3.32 (s, 3H), 3.28-3.20 (m, 1H), 3.18-3.05 (m, 1H), 2.98-2.84 (m, 1H), 2.77-2.58 (m, 2H), 2.49-2.46 (m, 1H), 2.38-2.23 (m, 2H), 2.21-2.08 (m, 4H), 2.05-1.93 (m, 3H), 1.93-1.83 (m, 2H), 1.82-1.57 (m, 10H), 1.43-1.33 (m, 18H), 1.02-0.87 (m, 4H); LC/MS (ESI, m/z): [(M+H)]+=907.5.

Step 2—(2S)-2-[[(5S,8S,10aR)-5-[(tert-butoxycarbonyl)amino]-6-oxo-3-[2-[(1r,4r)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoic acid. To a stirred solution of tert-butyl (2S)-2-[[(5S,8S,10aR)-5-[(tert-butoxycarbonyl)amino]-6-oxo-3-[2-[(1r,4r)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-4-carbamoylbutanoate (370.00 mg, 0.408 mmol) in DCM (8.00 mL) was added TFA (2.00 mL) dropwise at rt under nitrogen atmosphere. The resulting mixture was stirred for 4 h at rt under nitrogen atmosphere. On completion, the reaction mixture was concentrated under reduced pressure. Next, the residue was re-dissolved with DCM (7.00 mL). Then to the solution was added TEA (0.287 mL, 2.06 mmol) dropwise and Boc2O (107.98 mg, 0.495 mmol) at 0° C. Then the reaction mixture was stirred overnight at rt under nitrogen atmosphere. On completion, the reaction mixture was concentrated under reduced pressure. The residue was purified by reverse phase flash chromatography (Column: WelFlash™ C18-I, 20-40 ?m, 330 g; Eluent A: Water (plus 10 mmol/L FA); Eluent B: ACN; Gradient: 35%-55% B in 15 min; Flow rate: 80 mL/min; Detector: 220/254 nm; desired fractions were collected at 47% B) and concentrated under reduced pressure to afford the title compound (180 mg, 51.30%) as a white solid. ¹H NMR (300 MHz, DMSO-d6) δ 12.27 (broad, 1H), 11.06 (s, 1H), 8.23-8.16 (m, 1H), 7.22-7.14 (m, 1H), 7.01-6.96 (m, 2H), 6.80 (d, J=8.1 Hz, 1H), 6.73-6.67 (m, 1H), 6.55 (d, J=7.1 Hz, 1H), 5.32 (dd, J=12.7, 5.3 Hz, 1H), 4.43-4.32 (m, 2H), 4.18-4.01 (m, 2H), 3.68-3.64 (m, 2H), 3.31 (s, 3H), 3.24-3.08 (m, 2H), 3.04-2.80 (m, 2H), 2.77-2.57 (m, 2H), 2.47-2.43 (m, 1H), 2.39-2.23 (m, 2H), 2.21-2.09 (m, 4H), 2.04-1.87 (m, 3H), 1.86-1.54 (m, 10H), 1.37 (s, 9H), 1.01-0.84 (m, 4H); LC/MS (ESI, m/z): [(M+H)]+=851.3.

### (2S,4R)-1-[(2S)-2-amino-3,3-dimethylbutanoyl]-4-hydroxy-N-[1-[4-(4-methyl-1,3-thiazol-5-yl)phenyl]cyclopropyl]pyrrolidine-2-carboxamide (Intermediate CQ) (CAS #2502205-75-4)

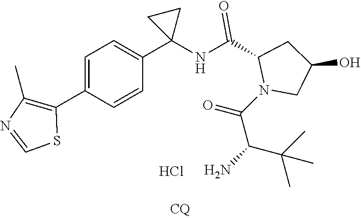 [SMILES: Cc1ncsc1-c1ccc(C2(NC(=O)[C@@H]3C[C@@H](O)CN3C(=O)[C@@H](N)C(C)(C)C)CC2)cc1 ; Cl]

### (2S,4R)-1-[(2S)-2-(4-[3-[(2S)-2-amino-4-carbamoylbutoxy]-2-chlorophenyl]butanamido)-3,3-dimethylbutanoyl]-4-hydroxy-N-[1-[4-(4-methyl-1,3-thiazol-5-yl)phenyl]cyclopropyl]pyrrolidine-2-carboxamide (Intermediate CR)

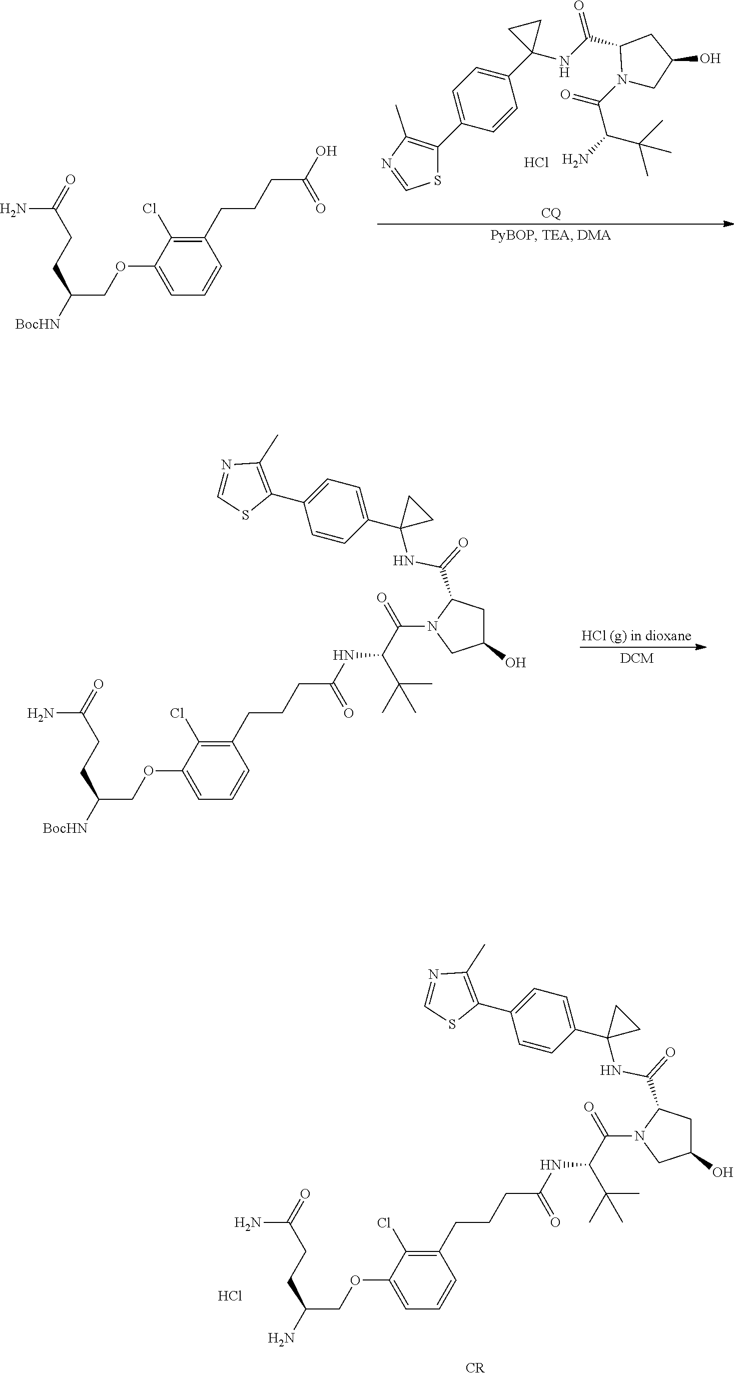 [SMILES: CC(C)(C)OC(=O)N[C@@H](CCC(N)=O)COc1cccc(CCCC(=O)O)c1Cl ; Cc1ncsc1-c1ccc(C2(NC(=O)[C@@H]3C[C@@H](O)CN3C(=O)[C@@H](N)C(C)(C)C)CC2)cc1 ; Cl ; Cc1ncsc1-c1ccc(C2(NC(=O)[C@@H]3C[C@@H](O)CN3C(=O)[C@@H](NC(=O)CCCc3cccc(OC[C@H](CCC(N)=O)NC(=O)OC(C)(C)C)c3Cl)C(C)(C)C)CC2)cc1 ; Cl ; Cc1ncsc1-c1ccc(C2(NC(=O)[C@@H]3C[C@@H](O)CN3C(=O)[C@@H](NC(=O)CCCc3cccc(OC[C@@H](N)CCC(N)=O)c3Cl)C(C)(C)C)CC2)cc1]

Step 1—tert-butyl N-[(2S)-4-carbamoyl-1-[2-chloro-3-(3-[[(2S)-1-[(2S,4R)-4-hydroxy-2-([1-[4-(4-methyl-1,3-thiazol-5-yl)phenyl]cyclopropyl]carbamoyl)pyrrolidin-1-yl]-3,3-dimethyl-1-oxobutan-2-yl]carbamoyl]propyl)phenoxy]butan-2-yl]carbamate. To a stirred solution of 4-[3-[(2S)-2-[(tert-butoxycarbonyl)amino]-4-carbamoylbutoxy]-2-chlorophenyl]butanoic acid (620.00 mg, 1.45 mmol, Intermediate E) and (2S,4R)-1-[(2S)-2-amino-3,3-dimethylbutanoyl]-4-hydroxy-N-[1-[4-(4-methyl-1,3-thiazol-5-yl)phenyl]cyclopropyl]pyrrolidine-2-carboxamide hydrochloride (926.55 mg, 1.88 mmol, Intermediate CQ) in DMA (10.00 mL) were added TEA (0.804 mL, 5.782 mmol) and HATU (714.52 mg, 1.88 mmol) in portions at 25° C. under nitrogen atmosphere. The resulting mixture was stirred for 2 h at 25° C. under nitrogen atmosphere. On completion, the reaction liquid was purified by reverse phase flash (Column: Spherical C18 Column, 20-40 um, 120 g; Mobile Phase A: Water (0.05% NH₄HCO₃), Mobile Phase B: ACN; Flow rate: 60 mL/min; Gradient: 30% B to 50% B in 25 min, 254 nml; the fractions containing the desired product were collected at 40% B) to afford the title compound (930 mg, 74% yield) as a white solid. ¹H NMR (400 MHz, Methanol-d4) δ 8.88 (d, J=2.6 Hz, 1H), 7.43-7.32 (m, 4H), 7.22-7.16 (m, 1H), 6.99-6.91 (m, 2H), 4.67-4.65 (m, 1H), 4.61-4.57 (m, 1H), 4.53-4.51 (m, 1H), 4.04-3.91 (m, 3H), 3.85-3.80 (m, 1H), 2.84-2.76 (m, 2H), 2.47 (s, 3H), 2.45-2.33 (m, 4H), 2.26-2.18 (m, 1H), 2.10-2.02 (m, 3H), 1.98-1.81 (m, 3H), 1.47 (s, 9H), 1.40-1.28 (m, 4H), 1.07 (s, 9H); LC/MS (ESI, m/z): [(M+1)]+=867.2.

Step 2—(2S,4R)-1-[(2S)-2-(4-[3-[(2S)-2-amino-4-carbamoylbutoxy]-2-chlorophenyl]butanamido)-3,3-dimethylbutanoyl]-4-hydroxy-N-[1-[4-(4-methyl-1,3-thiazol-5-yl)phenyl]cyclopropyl]pyrrolidine-2-carboxamide hydrochloride. To a stirred solution of tert-butyl N-[(2S)-4-carbamoyl-1-[2-chloro-3-(3-[[(2S)-1-[(2S,4R)-4-hydroxy-2-([1-[4-(4-methyl-1,3-thiazol-5-yl)phenyl]cyclopropyl]carbamoyl)pyrrolidin-1-yl]-3,3-dimethyl-1-oxobutan-2-yl]carbamoyl]propyl)phenoxy]butan-2-yl]carbamate (930.00 mg, 1.072 mmol) in dioxane (10.00 mL) was added HCl (gas) in 1,4-dioxane (3.00 mL) dropwise at 25° C. under nitrogen atmosphere. The resulting mixture was stirred for 2 h at 25° C. under nitrogen atmosphere. On completion, the reaction mixture was concentrated under reduced pressure to give the title compound (940 mg) as a white solid. ¹H NMR (300 MHz, DMSO-d6) δ 9.06 (s, 1H), 8.88-8.83 (m, 1H), 8.39-8.33 (m, 3H), 8.02 (d, J=9.2 Hz, 1H), 7.51-7.46 (m, 1H), 7.38-7.31 (m, 4H), 7.30-7.19 (m, 2H), 7.10-7.02 (m, 1H), 7.01-6.90 (m, 2H), 4.58 (d, J=9.3 Hz, 1H), 4.45-4.39 (m, 4H), 4.22-4.16 (m, 3H), 3.68-3.65 (m, 1H), 2.75-2.70 (m, 2H), 2.46 (s, 3H), 2.37-2.20 (m, 4H), 2.04-1.94 (m, 3H), 1.85-1.76 (m, 2H), 1.29-1.14 (m, 4H), 0.96 (s, 9H); LC/MS (ESI, m/z): [(M+1)]+=767.2.

### (5S,8S,10aR)-5-[(tert-butoxycarbonyl)amino]-6-oxo-3-(2,2,2-trifluoroacetyl)-octahydropyrrolo[1,2-a][1,5]diazocine-8-carboxylic acid (Intermediate CS)

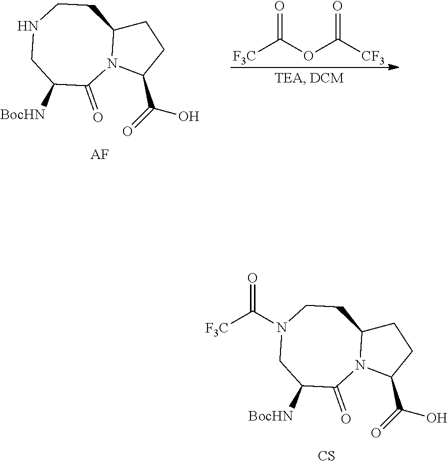

To a stirred mixture of (5S,8S,10aR)-5-[(tert-butoxycarbonyl)amino]-6-oxo-octahydro-1H-pyrrolo[1,2-a][1,5]diazocine-8-carboxylic acid (300.00 mg, 0.916 mmol, Intermediate AF) and TEA (0.382 mL, 2.75 mmol) in DCM (7.00 mL) was added trifluoroacetic anhydride (384.93 mg, 1.833 mmol) dropwise at 0° C. under air atmosphere. The resulting mixture was stirred for 2 h at rt under air atmosphere. On completion, the reaction mixture was extracted with DCM (3×10 mL). The combined organic layers were washed with DCM (3×100 mL), and dried over anhydrous Na₂SO₄. After filtration, the filtrate was concentrated under reduced pressure. The residue was purified by reverse phase flash chromatography (Column: WelFlash™ C18-I, 20-40 m, 120 g; Eluent A: Water (plus 10 mmol/L NH4HCO3); Eluent B: ACN; Gradient: 25%-55% B in 25 min; Flow rate: 60 mL/min; Detector: 220/254 nm; desired fractions were collected at 30% B) and concentrated under reduced pressure to afford the title compound (125 mg, 32% yield) as a white solid. ¹H NMR (300 MHz, Methanol-d4) δ 4.75-4.68 (m, 1H), 4.42-4.34 (m, 1H), 4.33-4.25 (m, 1H), 3.92-3.72 (m, 3H), 3.64-3.55 (m, 1H), 2.42-2.30 (m, 1H), 2.22-1.87 (m, 4H), 1.83-1.74 (m, 1H), 1.44 (s, 9H); LC/MS (ESI, m/z): [(M+H)]+=424.2.

### (2S,4R)-1-[(2S)-2-(5-[3-[(2S)-2-amino-4-carbamoylbutoxy]-2-chlorophenyl]pentanamido)-3,3-dimethylbutanoyl]-4-hydroxy-N-[1-[4-(4-methyl-1,3-thiazol-5-yl)phenyl]cyclopropyl]pyrrolidine-2-carboxamide (Intermediate CT)

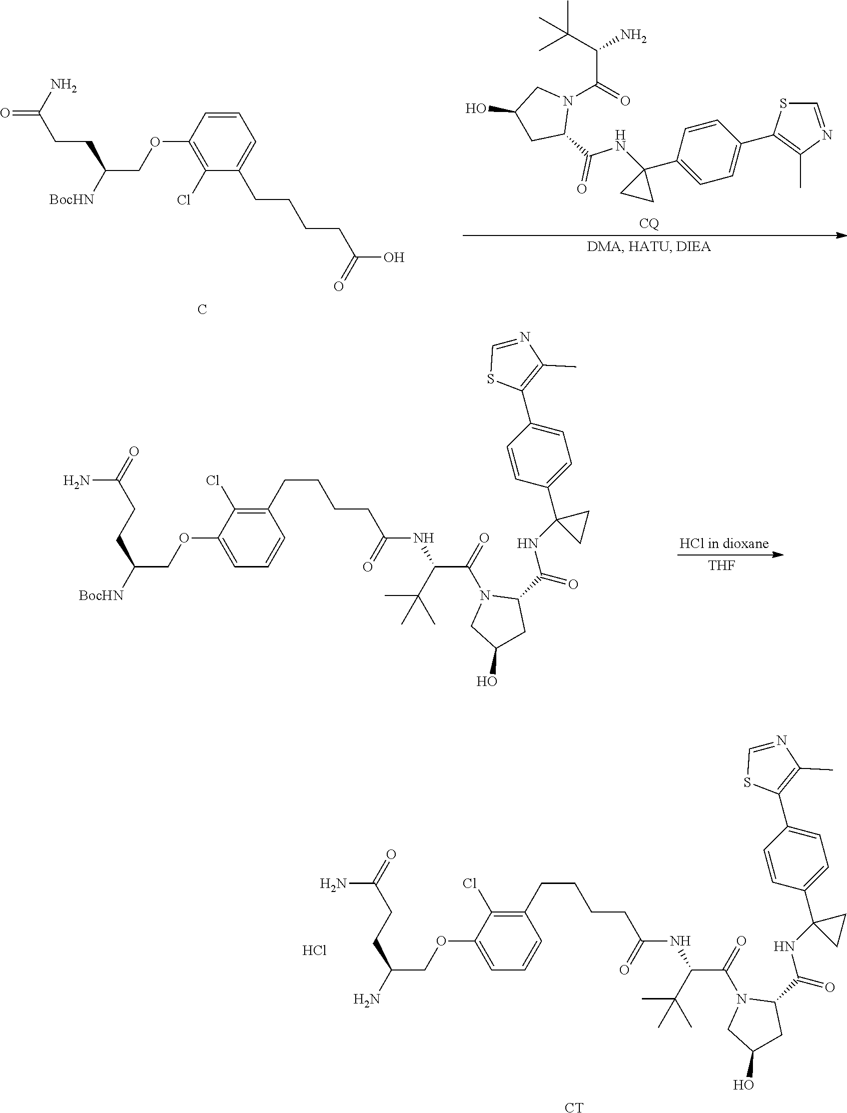

Step 1—Tert-butyl N-[(2S)-4-carbamoyl-1-[2-chloro-3-(4-[[(2S)-1-[(2S,4R)-4-hydroxy-2-([1-[4-(4-methyl-1,3-thiazol-5-yl)phenyl]cyclopropyl]carbamoyl)pyrrolidin-1-yl]-3,3-dimethyl-1-oxobutan-2-yl]carbamoyl]butyl)phenoxy]butan-2-yl]carbamate. To a stirred mixture of 5-[3-[(2S)-2-[(tert-butoxycarbonyl)amino]-4-carbamoylbutoxy]-2-chlorophenyl]pentanoic acid (50.00 mg, 0.113 mmol, Intermediate C) and (2S,4R)-1-[(2S)-2-amino-3,3-dimethylbutanoyl]-4-hydroxy-N-[1-[4-(4-methyl-1,3-thiazol-5-yl)phenyl]cyclopropyl]pyrrolidine-2-carboxamide (56.70 mg, 0.124 mmol, Intermediate CQ) in DMA (1.00 mL) were added TEA (0.047 mL, 0.339 mmol) and HATU (51.51 mg, 0.135 mmol) in turns at rt under nitrogen atmosphere. The resulting mixture was stirred for 16 h at rt under nitrogen atmosphere. The residue was purified by reverse phase flash chromatography (Column: WelFlash™ C18-I, 20-40 um, 120 g; Eluent A: Water (plus 10 mmol/L NLHCO₃); Eluent B: ACN; Gradient: 25%-55% B in 25 min; Flow rate: 50 mL/min; Detector: 220/254 nm; desired fractions were collected at 50% B) and concentrated under reduced pressure to afford the title compound (73.36 mg, 74% yield) as a white solid. ¹H NMR (300 MHz, DMSO-d6) δ 8.96 (s, 1H), 879 (s, 1H), 7.97-7.93 (m, 1H), 7.32-7.28 (m, 5H), 7.24-7.12 (m, 1H), 6.96-6.92 (m, 2H), 6.85-6.71 (m, 2H), 5.15-5.11 (m, 1H), 4.57-4.53 (m, 1H), 4.44-4.33 (m, 2H), 3.94-3.90 (m, 2H), 3.82-3.71 (m, 2H), 3.67-3.63 (m, 2H), 2.72-2.68 (m, 2H), 2.43 (s, 3H), 2.38-2.27 (m, 1H), 2.17-2.13 (m, 3H), 2.02-1.98 (m, 1H), 1.94-1.80 (m, 2H), 1.68-1.46 (m, 4H), 1.38 (s, 9H), 1.29-1.09 (m, 4H), 0.94 (s, 9H); LC/MS (ESI, m/z): [(M+H)]+=881.4.

Step 2—(2S,4R)-1-[(2S)-2-(5-[3-[(2S)-2-amino-4-carbamoylbutoxy]-2-chlorophenyl]pentanamido)-3,3-dimethylbutanoyl]-4-hydroxy-N-[1-[4-(4-methyl-1,3-thiazol-5-yl)phenyl]cyclopropyl]pyrrolidine-2-carboxamide hydrochloride. To a stirred solution of tert-butyl N-[(2S)-4-carbamoyl-1-[2-chloro-3-(4-[[(2S)-1-[(2S,4R)-4-hydroxy-2-([1-[4-(4-methyl-1,3-thiazol-5-yl)phenyl]cyclopropyl]carbamoyl)pyrrolidin-1-yl]-3,3-dimethyl-1-oxobutan-2-yl]carbamoyl]butyl)phenoxy]butan-2-yl]carbamate (1.10 g, 1.25 mmol) in THF (10.00 mL) was added HCl (gas) dropwise at rt. The resulting mixture was stirred for 2 h at rt under nitrogen atmosphere. The resulting mixture was concentrated under reduced pressure to afford the title compound (900 mg) as a white solid. ¹H NMR (400 MHz, DMSO-d6) δ 9.01 (s, 1H), 8.82 (s, 1H), 8.38-8.19 (m, 3H), 7.97-7.93 (m, 1H), 7.46 (s, 1H), 7.37-7.27 (m, 4H), 7.27-7.23 (m, 1H), 7.06-7.02 (m, 1H), 7.01-6.97 (m, 1H), 6.96-6.92 (m, 1H), 4.57-4.53 (m, 1H), 4.41-4.37 (m, 2H), 4.25-4.21 (m, 1H), 4.15-4.11 (m, 1H), 3.70-3.58 (m, 2H), 3.57-3.53 (m, 1H), 2.74-2.66 (m, 2H), 2.45 (s, 3H), 2.38-2.24 (m, 3H), 2.22-2.14 (m, 1H), 2.02-1.92 (m, 3H), 1.89-1.85 (m, 2H), 1.55-1.51 (m, 4H), 1.29-1.08 (m, 4H), 0.94 (s, 9H); LC/MS (ESI, m/z): [(M+H)]+=781.3.

### (2S)-2-amino-N-[(3-fluoro-4-methanesulfonylphenyl)methyl]pentanediamide (Intermediate CU)

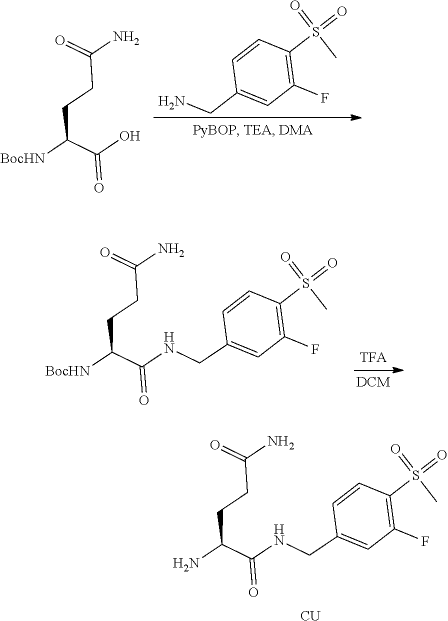

Step 1—Tert-butyl N-[(1S)-3-carbamoyl-1-[[(3-fluoro-4-methanesulfonylphenyl)methyl]carbamoyl]propyl]carbamate. To a stirred mixture of (2S)-2-[(tert-butoxycarbonyl)amino]-4-carbamoylbutanoic acid (250.00 mg, 1.02 mmol, CAS #13726-85-7) and PyBOP (686.78 mg, 1.32 mmol) in DMA (3.00 mL) were added TEA (0.423 mL, 3.05 mmol) and 1-(3-fluoro-4-methanesulfonylphenyl)methanamine (226.95 mg, 1.12 mmol, CAS #: 1425092-76-7) in turns at rt under nitrogen atmosphere. The resulting mixture was stirred for 2 h at rt under nitrogen atmosphere. The residue was purified by reverse phase flash chromatography (Column: WelFlash™ C18-I, 20-40 um, 120 g; Eluent A: Water (plus 10 mmol/L NH4HCO3); Eluent B: ACN; Gradient: 25%-55% B in 25 min; Flow rate: 60 mL/min; Detector: 220/254 nm; desired fractions were collected at 42% B) and concentrated under reduced pressure to afford the title compound (300 mg, 69% yield) as a white solid. ¹H NMR (400 MHz, DMSO-d6) δ 8.54-8.50 (m, 1H), 7.80-7.76 (m, 1H), 7.39-7.33 (m, 2H), 7.30-7.25 (m, 1H), 7.10-7.06 (m, 1H), 6.80-6.75 (m, 1H), 4.40-4.36 (m, 2H), 3.91-3.87 (m, 1H), 3.30 (s, 3H), 2.14-2.10 (m, 2H), 1.93-1.80 (m, 1H), 1.76-1.72 (m, 1H), 1.41 (s, 9H); LC/MS (ESI, m/z): [(M+H)]+=432.2.

Step 2—(2S)-2-amino-N-[(3-fluoro-4-methanesulfonylphenyl)methyl]pentanediamide trifluoroacetate. To a stirred solution of tert-butyl N-[(1S)-3-carbamoyl-1-[[(3-fluoro-4-methanesulfonylphenyl)methyl]carbamoyl]propyl]carbamate (300.00 mg, 0.695 mmol) in DCM (10.00 mL) was added TFA (2.00 mL) dropwise at 0° C. under nitrogen atmosphere. The resulting mixture was stirred for 2 h at rt under nitrogen atmosphere. The resulting mixture was concentrated under reduced pressure to afford the title compound (272 mg, 91% yield) as alight yellow oil. ¹H NMR (300 MHz, DMSO-d6) δ 9.12-9.08 (m, 1H), 8.24-8.20 (m, 1H), 7.92-7.78 (m, 1H), 7.48-7.26 (m, 4H), 7.13 (s, 1H), 6.99-6.95 (m, 1H), 4.44-4.40 (m, 2H), 3.91-3.87 (m, 1H), 3.32 (s, 3H), 2.26-2.11 (m, 2H), 2.03-1.85 (m, 2H); LC/MS (ESI, m/z): [(M+H)]+=332.1.

### (2S)-2-amino-N-[(2-fluoro-4-methanesulfonylphenyl)methyl]pentanediamide (Intermediate CV)

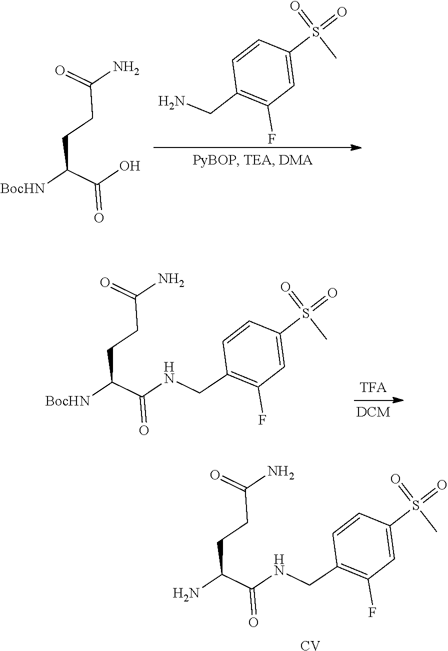

Step 1—Tert-butyl N-[(1S)-3-carbamoyl-1-[[(2-fluoro-4-methanesulfonylphenyl)methyl]carbamoyl]propyl]carbamate. To a stirred mixture of (2S)-2-[(tert-butoxycarbonyl)amino]-4-carbamoylbutanoic acid (250.00 mg, 1.02 mmol) and PyBOP (686.78 mg, 1.32 mmol) in DMA (3.00 mL) were added TEA (0.423 mL, 3.05 mmol) and 1-(2-fluoro-4-methanesulfonylphenyl)methanamine (226.95 mg, 1.12 mmol, CAS #: 1002556-98-0) in turns at rt under nitrogen atmosphere. The resulting mixture was stirred for 2 h at rt under nitrogen atmosphere. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 42% B) and concentrated under reduced pressure to afford the title compound (300 mg, 69% yield) as a white solid. ¹H NMR (400 MHz, DMSO-d6) δ 8.54-8.50 (m, 1H), 7.80-7.76 (m, 1H), 7.39-7.33 (m, 2H), 7.30-7.25 (m, 1H), 7.10-7.06 (m, 1H), 6.80-6.75 (m, 1H), 4.40-4.36 (m, 2H), 3.91-3.87 (m, 1H), 3.32 (s, 3H), 2.14-2.10 (m, 2H), 1.93-1.80 (m, 1H), 1.76-1.72 (m, 1H), 1.41 (s, 9H); LC/MS (ESI, m/z): [(M+H)]+=432.15.

Step 2—(2S)-2-amino-N-[(2-fluoro-4-methanesulfonylphenyl)methyl]pentanediamide trifluoroacetate. To a stirred solution of tert-butyl N-[(1S)-3-carbamoyl-1-[[(2-fluoro-4-methanesulfonylphenyl)methyl]carbamoyl]propyl]carbamate (300.00 mg, 0.695 mmol) in DCM (10.00 mL) was added TFA (2.00 mL) dropwise at 0° C. under nitrogen atmosphere. The resulting mixture was stirred for 2 h at rt under nitrogen atmosphere. The resulting mixture was concentrated under reduced pressure to afford the title compound (272 mg) as a light yellow oil. ¹H NMR (300 MHz, DMSO-d6) δ 9.12-9.08 (m, 1H), 8.24-8.20 (m, 1H), 7.92-7.78 (m, 1H), 7.48-7.26 (m, 4H), 7.13 (s, 1H), 6.99-6.95 (m, 1H), 4.44-4.40 (m, 2H), 3.91-3.87 (m, 1H), 3.32 (s, 3H), 2.26-2.11 (m, 2H), 2.03-1.85 (m, 2H); LC/MS (ESI, m/z): [(M+H)]+=332.1.

### (2S)-2-amino-N-[(1R)-1-(4-methanesulfonylphenyl)ethyl]pentanediamide (Intermediate CW)

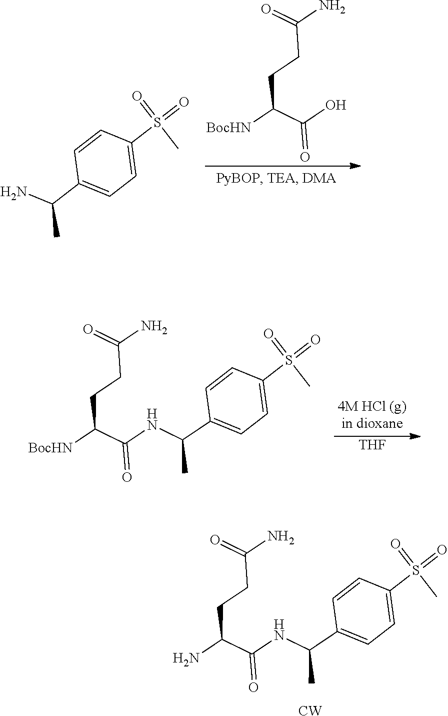

Step 1—Tert-butyl N-[(1S)-3-carbamoyl-1-[[(1R)-1-(4-methanesulfonylphenyl)ethyl]carbamoyl]propyl]carbamate. To a stirred mixture of (2S)-2-[(tert-butoxycarbonyl)amino]-4-carbamoylbutanoic acid (250.00 mg, 1.015 mmol) and in DMA (4.00 mL) were added TEA (308.18 mg, 3.046 mmol) and (1R)-1-(4-methanesulfonylphenyl)ethanamine (0.423 mL, 1.218 mmol, CAS #: 1038393-47-3) in turns at rt under nitrogen atmosphere. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 m, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 42% B) and concentrated under reduced pressure to afford the title compound (290 mg, 69% yield) as a white solid. ¹H NMR (300 MHz, Chloroform-d) δ 8.35-8.32 (m, 2H), 7.92-7.86 (m, 2H), 7.70-7.62 (m, 2H), 7.53 (s, 1H), 6.95 (s, 1H), 5.05-4.98 (m, 1H), 3.94-3.75 (m, 1H), 3.57 (s, 3H), 2.53-2.49 (m, 4H), 1.46-1.41 (m, 3H), 1.40 (s, 9H); LC/MS (ESI, m/z): [(M+H)]+=428.2.

Step 2—(2S)-2-amino-N-[(1R)-1-(4-methanesulfonylphenyl)ethyl]pentanediamide hydrochloride. To a stirred solution of tert-butyl N-[(1S)-3-carbamoyl-1-[[(1R)-1-(4-methanesulfonylphenyl)ethyl]carbamoyl]propyl]carbamate (290.00 mg, 0.678 mmol) in THF (8.00 mL) was added 4 M HCl (gas) in 1,4-dioxane (8.00 mL) dropwise at rt. The resulting mixture was stirred for 2 h at rt under nitrogen atmosphere. On completion, the resulting mixture was concentrated under reduced pressure. The residue was triturated with Et2O (30.00 mL) to afford the title compound (200 mg, 81% yield) as a white solid. ¹H NMR (300 MHz, Chloroform-d) δ 9.34-9.19 (m, 1H), 8.35-8.30 (m, 2H), 7.92-7.86 (m, 2H), 7.70-7.62 (m, 2H), 7.53 (s, 1H), 6.95 (s, 1H), 5.05-4.99 (m, 1H), 3.94-3.76 (m, 1H), 3.57 (s, 3H), 2.53-2.49 (m, 4H), 1.47-1.40 (m, 3H); LC/MS (ESI, m/z): [(M+H)]+=328.1.

### (2S,4R)-1-[(2S)-2-(5-[3-[(2R)-2-amino-4-carbamoylbutoxy]-2-chlorophenyl]pentanamido)-3,3-cimethylbutanoyl]-4-hydroxy-N-[(1S)-1-[4-(4-methyl-1,3-thiazol-5-yl)phenyl]ethyl]pyrrolidine-2-carboxamide (Intermediate CX)

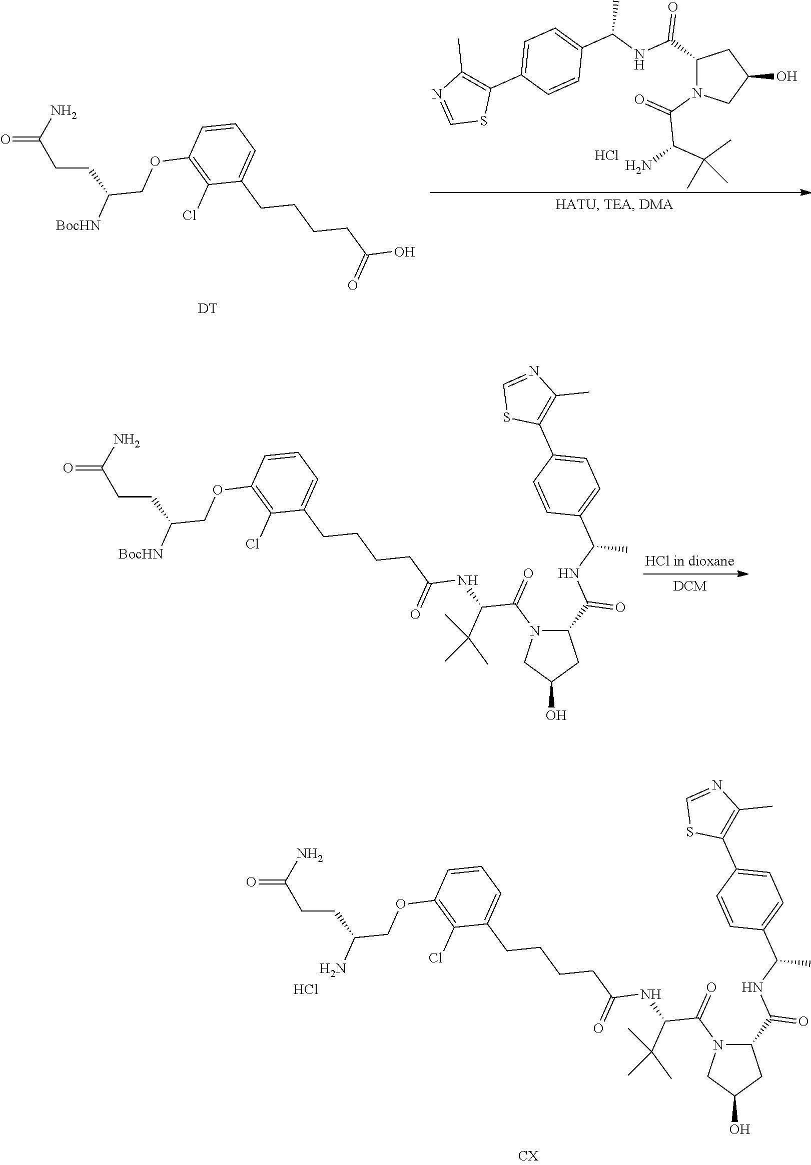

Step 1—Tert-butyl N-[(2R)-4-carbamoyl-1-[2-chloro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate. To a stirred solution of 5-[3-[(2R)-2-[(tert-butoxycarbonyl)amino]-4-carbamoylbutoxy]-2-chlorophenyl]pentanoic acid (400.00 mg, 0.903 mmol, Intermediate DT) and (2S,4R)-1-[(2S)-2-amino-3,3-dimethylbutanoyl]-4-hydroxy-N—[(S)-1-[4-(4-methyl-1,3-thiazol-5-yl)phenyl]ethyl]pyrrolidine-2-carboxamide hydrochloride (521.30 mg, 1.084 mmol, CAS #1448189-80-7) in DMA (6.00 mL) were added TEA (274.14 mg, 2.709 mmol) and HATU (412.04 mg, 1.084 mmol) in turns at 0° C. under nitrogen atmosphere. The resulting mixture was stirred for 1 h at rt under nitrogen atmosphere. On completion, the reaction mixture was concentrated under reduced pressure. The residue was purified by reverse flash chromatography (Column, Welflash™ C18-1, 20-40 um, 330 g; Eluent A: Water (plus 10 mmol/L FA); Eluent B: ACN; Gradient 30% to 60% B in 30 min; Flow rate: 80 mL/min; Detector: 220/254 nm; desired fractions were collected at 46% B) and concentrated under reduced pressure to afford the title compound (900 mg, 92% yield) as a white solid. ¹H NMR (300 MHz, Chloroform-d) δ 8.71 (s, 1H), 7.75-7.46 (m, 1H), 7.46-7.34 (m, 4H), 7.16-7.04 (m, 1H), 6.89-6.81 (m, 1H), 6.80-6.70 (m, 1H), 6.42-6.26 (m, 2H), 5.75-6.65 (m, 1H), 5.34-5.27 (m, 1H), 5.24-5.15 (m, 1H), 5.15-5.05 (m, 1H), 4.75-4.65 (m, 1H), 4.62-4.49 (m, 2H), 4.12-4.00 (m, 4H), 3.65-3.58 (m, 1H), 2.79-2.69 (m, 2H), 2.55 (s, 3H), 2.47-2.42 (m, 1H), 2.39-2.30 (m, 2H), 2.28-2.20 (m, 2H), 2.14-2.03 (m, 3H), 1.70-1.56 (m, 4H), 1.52-1.48 (m, 3H), 1.47 (s, 9H), 1.05 (s, 9H); LC/MS (ESI, m/z): [(M+H)]+=869.4.

Step 2—(2S,4R)-1-[(2S)-2-(5-[3-[(2R)-2-amino-4-carbamoylbutoxy]-2-chlorophenyl]pentanamido)-3,3-Dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2R)-4-carbamoyl-1-[2-chloro-3-(4-[[(2S)-1-[(2S,4R)-4-hydroxy-2-[[(1S)-1-[4-(4-methyl-1, 3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamate (917.00 mg, 1.055 mmol) in DCM (9 mL) was added a solution of 4 M HCl (gas) in 1,4-dioxane (7 mL) dropwise at rt under nitrogen atmosphere. The resulting mixture was stirred for 2 h at rt under nitrogen atmosphere. On completion, the reaction mixture was concentrated under reduced pressure. The residue was purified by trituration with Et₂O (10 mL). The precipitated solids was concentrated under reduced pressure to afford the title compound (860 mg, 93% yield) as a white solid. ¹H NMR (300 MHz, Methanol-d4) δ 10.01 (s, 1H), 7.66-7.43 (m, 4H), 7.27-7.11 (m, 1H), 7.06-6.89 (m, 2H), 5.06-4.97 (m, 1H), 4.65-4.53 (m, 2H), 4.49-4.40 (m, 1H), 4.34-4.24 (m, 1H), 4.20-4.09 (m, 1H), 3.94-3.82 (m, 1H), 3.77-3.70 (m, 1H), 3.67 (s, 3H), 3.52-3.40 (m, 1H), 2.82-2.71 (m, 1H), 2.62 (s, 3H), 2.56-2.47 (m, 1H), 2.41-2.26 (m, 2H), 2.23-2.07 (m, 2H), 1.96-1.86 (m, 1H), 1.71-1.63 (m, 2H), 1.63-1.57 (m, 2H), 1.52 (d, J=7.0 Hz, 3H), 1.04 (s, 9H); LC/MS (ESI, m/z): [(M+H)]+=769.4.

### (2S,4R)-1-[(2S)-2-[2-(4-[3-[(2S)-2-amino-4-carbamoylbutoxy]-2-chlorophenyl]piperidin-1-yl)acetamido]-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate CY)

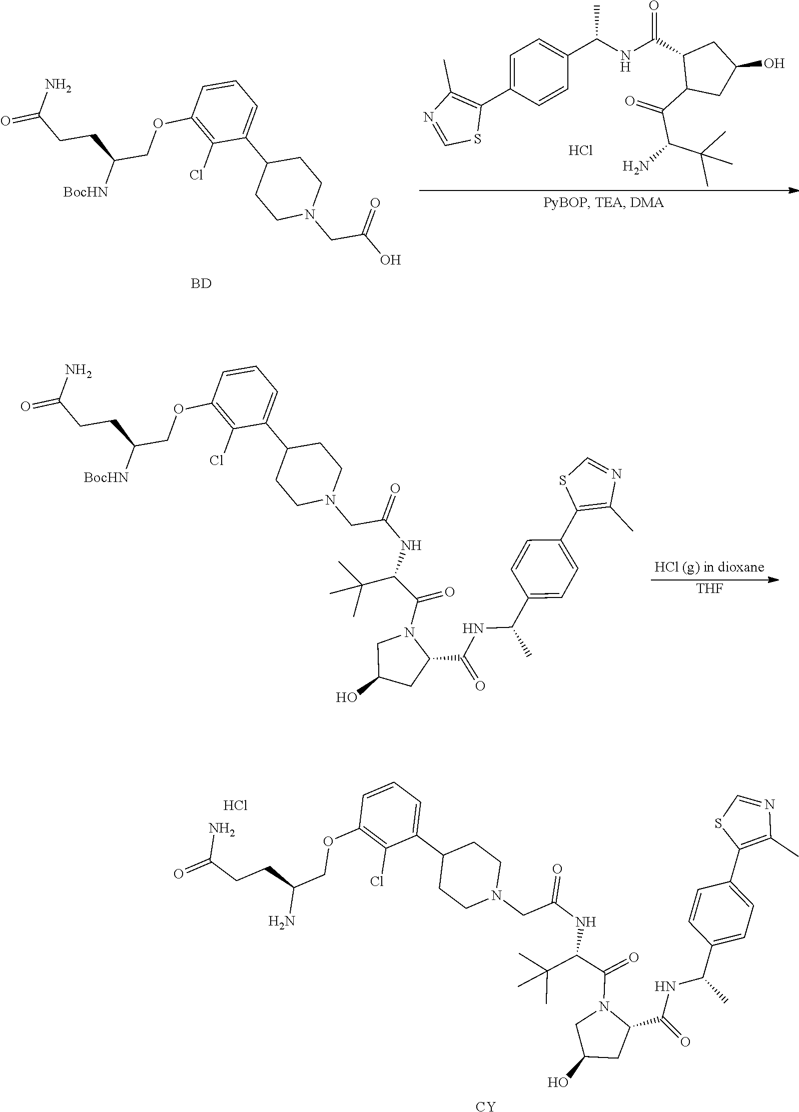

Step 1—Tert-butyl N-[(2S)-4-carbamoyl-1-[2-chloro-3-[1-([[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]methyl)piperidin-4-yl]phenoxy]butan-2-yl]carbamate. To a stirred solution of (4-[3-[(2S)-2-[(tert-butoxycarbonyl)amino]-4-carbamoylbutoxy]-2-chlorophenyl]piperidin-1-yl)acetic acid (260.00 mg, 0.54 mmol, Intermediate BD) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (335.95 mg, 0.698 mmol, CAS #1448189-80-7) in DMA (10.00 mL) were added PyBOP (363.42 mg, 0.698 mmol) and TEA (217.44 mg, 2.15 mmol) dropwise at 25° C. under nitrogen atmosphere. The resulting mixture was stirred for 2 h at 25° C. under nitrogen atmosphere. On completion, the reaction liquid was purified by reverse phase flash (Column: Spherical C18 Column, 20-40 um, 120 g; Mobile Phase A: Water (10 mmol/L NH4HCO3), Mobile Phase B: ACN; Flow rate: 60 mL/min; Gradient: 35% B to 55% B in 25 min, 254 nm, the fractions containing the desired product were collected at 48% B) to afford the title compound (460 mg, 94% yield) as a white solid. ¹H NMR (300 MHz, Methanol-d4) δ 8.88 (s, 1H), 7.47-7.38 (m, 4H), 7.30-7.18 (m, 1H), 7.05-6.99 (m, 1H), 6.97-6.91 (m, 1H), 5.08-4.97 (m, 1H), 4.64-4.50 (m, 1H), 4.48-4.36 (m, 1H), 4.05-3.83 (m, 4H), 3.81-3.70 (m, 1H), 3.21-2.93 (m, 6H), 2.48 (s, 3H), 2.44-2.30 (m, 4H), 2.27-2.15 (m, 1H), 2.13-2.05 (m, 1H), 2.02-1.93 (m, 1H), 1.91-1.76 (m, 5H), 1.53 (d, J=7.0 Hz, 3H), 1.45 (s, 9H), 1.06 (s, 9H); LC/MS (ESI, m/z): [(M+H)]+=910.5.

Step 2—(2S,4R)-1-[(2S)-2-[2-(4-[3-[(2S)-2-amino-4-carbamoylbutoxy]-2-chlorophenyl]piperidin-1-yl)acetamido]-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-chloro-3-[1-([[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]methyl)piperidin-4-yl]phenoxy]butan-2-yl]carbamate (460.00 mg, 0.505 mmol) in DCM (10.00 mL) was added 4 M HCl (gas) in 1,4-dioxane (5.00 mL) dropwise at 25° C. under nitrogen atmosphere. The resulting mixture was stirred for 2 h at 25° C. under nitrogen atmosphere. On completion, the resulting mixture was concentrated under reduced pressure. The residue was triturated with Et₂O (10 mL) at rt for 2 h and filtrated to give the title compound (400 mg, 94% yield). ¹H NMR (300 MHz, DMSO-d6) δ 9.11-9.04 (m, 1H), 8.83-8.74 (m, 1H), 8.57-8.43 (m, 3H), 7.54-7.33 (m, 6H), 7.19-7.10 (m, 1H), 7.05-6.85 (m, 2H), 4.98-4.87 (m, 1H), 4.58-4.55 (m, 1H), 4.50-4.48 (m, 1H), 4.47-4.45 (m, 1H), 4.34-4.25 (m, 3H), 4.24-4.15 (m, 2H), 4.11-4.06 (m, 1H), 3.69-3.60 (m, 2H), 3.38-3.21 (m, 3H), 3.10-2.99 (m, 1H), 2.47 (s, 3H), 2.39-2.26 (m, 2H), 2.13-1.72 (m, 9H), 1.40 (d, J=6.9 Hz, 3H), 0.98 (s, 9H); LC/MS (ESI, m/z): [(M+H)]+=810.3.

### 4-[3-[(2S)-2-[(tert-butoxycarbonyl)amino]-4-carbamoylbutoxy]-2-fluorophenyl]butanoic acid (Intermediate CZ)

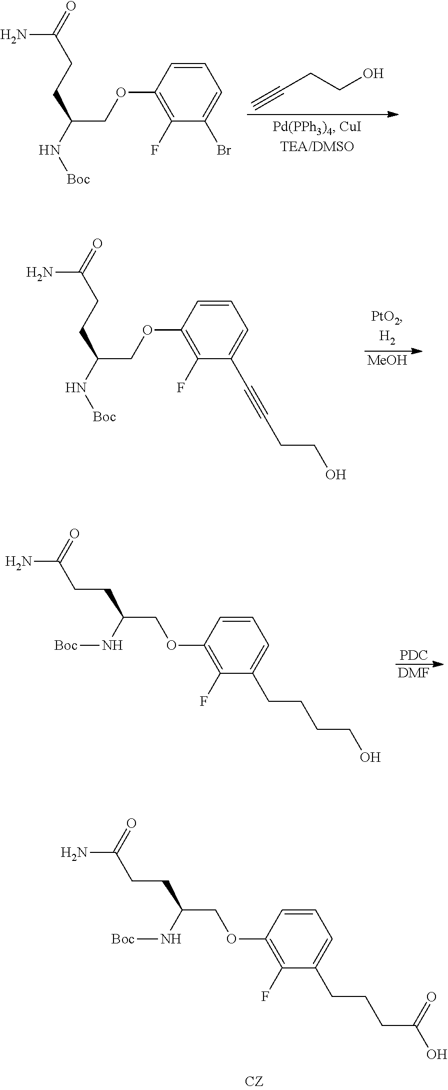

Step 1—Tert-butyl N-[(2S)-4-carbamoyl-1-[2-fluoro-3-(4-hydroxybut-1-yn-1-yl)phenoxy]butan-2-yl]carbamate. To a stirred mixture of tert-butyl N-[(2S)-1-(3-bromo-2-fluorophenoxy)-5-oxohexan-2-yl]carbamate (1.20 g, 2.97 mmol, synthesized via Step 1 of Intermediate Q) and 3-butyn-1-ol (624.15 mg, 8.91 mmol) in TEA (2.00 mL) and DMSO (6.00 mL) were added Pd(PPh₃)4 (343.00 mg, 0.30 mmol) and CuI (56.53 mg, 0.30 mmol) at rt under nitrogen atmosphere. The resulting mixture was stirred for 3 h at 80° C. under nitrogen atmosphere. On completion, the reaction mixture was filtered, the filter cake was washed with DCM (3×100 mL). The filtrate was concentrated under reduced pressure. The residue was purified by reverse phase flash chromatography (Column: WelFlash™ C18-I, 20-40 um, 330 g; Eluent A: Water (plus 10 mmol/L NH4HCO3); Eluent B: ACN; Gradient: 35%-75% B in 25 min; Flow rate: 80 mL/min; Detector: 220/254 nm; desired fractions were collected at 55% B) and concentrated under reduced pressure to afford the title compound (1.0 g, 85% yield) as a white solid. ¹H NMR (400 MHz, Methanol-d4) δ 7.12-7.07 (m, 1H), 7.06-6.95 (m, 2H), 4.06-3.98 (m, 2H), 3.93-3.85 (m, 1H), 3.79-3.71 (m, 2H), 2.72-2.64 (m, 2H), 2.39-2.30 (m, 2H), 2.05-1.98 (m, 1H), 1.82-1.80 (m, 1H), 1.46 (s, 9H); LC/MS (ESI, m/z): [(M+H)]+=395.2.

Step 2—Tert-butyl N-[(2S)-4-carbamoyl-1-[2-fluoro-3-(4-hydroxybutyl)phenoxy]butan-2-yl]carbamate. To a solution of tert-butyl N-[(2S)-4-carbamoyl-1-[2-fluoro-3-(4-hydroxybut-1-yn-1-yl)phenoxy]butan-2-yl]carbamate (1.00 g) in MeOH (20.00 mL) were added PtO2 (100.00 mg) and AcOH (0.10 mL) under nitrogen atmosphere in a 100 mL round-bottom flask. The mixture was hydrogenated at rt for 3 h under hydrogen atmosphere using a hydrogen balloon. On completion, the reaction mixture was filtered through a celite pad and concentrated under reduced pressure to afford the title compound (900 mg, 90% yield) as a off-white solid. ¹H NMR (300 MHz, Methanol-d4) δ 7.04-6.87 (m, 2H), 6.87-6.76 (m, 1H), 4.02-3.97 (m, 2H), 3.93-3.85 (m, 1H), 3.62-3.55 (m, 2H), 2.72-2.58 (m, 2H), 2.36-2.29 (m, 2H), 2.07-1.97 (m, 1H), 1.95-1.92 (m, 1H), 1.90-1.67 (m, 1H), 1.71-1.56 (m, 2H), 1.55-1.48 (m, 1H), 1.45 (s, 9H); LC/MS (ESI, m/z): [(M+H)]+=399.2.

Step 3—4-[3-[(2S)-2-[(tert-butoxycarbonyl)amino]-4-carbamoylbutoxy]-2-fluorophenyl]butanoic acid. To a stirred solution of tert-butyl N-[(2S)-4-carbamoyl-1-[2-fluoro-3-(4-hydroxybutyl)phenoxy]butan-2-yl]carbamate (100.00 mg, 0.251 mmol) in DMF (6.00 mL) was added PDC (472.05 mg, 1.26 mmol) at rt under nitrogen atmosphere. The resulting mixture was stirred for 16 h at rt under nitrogen atmosphere. On completion, the reaction mixture was diluted with water (100) and extracted with DCM (3×40 mL). The combined organic layers were washed with brine (2×30 mL), and dried over anhydrous Na2SO4. After filtration, the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 15%-45% B in 25 min; Flow rate: 80 mL/min; Detector: 220/254 nm; desired fractions were collected at 26% B) and concentrated under reduced pressure to afford the title compound (400 mg, 48% yield) as a yellow solid. ¹H NMR (300 MHz, Methanol-d4) δ 7.04-6.87 (m, 2H), 6.87-6.76 (m, 1H), 4.02-3.97 (m, 2H), 3.93-3.85 (m, 1H), 3.63-3.54 (m, 2H), 2.74-2.69 (m, 2H), 2.36-2.57 (m, 2H), 2.07-1.97 (m, 1H), 1.90-1.67 (m, 1H), 1.71-1.56 (m, 1H), 1.55-1.48 (m, 1H), 1.45 (s, 9H); LC/MS (ESI, m/z): [(M+H)]+=413.2.

### (2S,4R)-1-[(2S)-2-(4-[3-[(2S)-2-amino-4-carbamoylbutoxy]-2-fluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide (Intermediate DA)

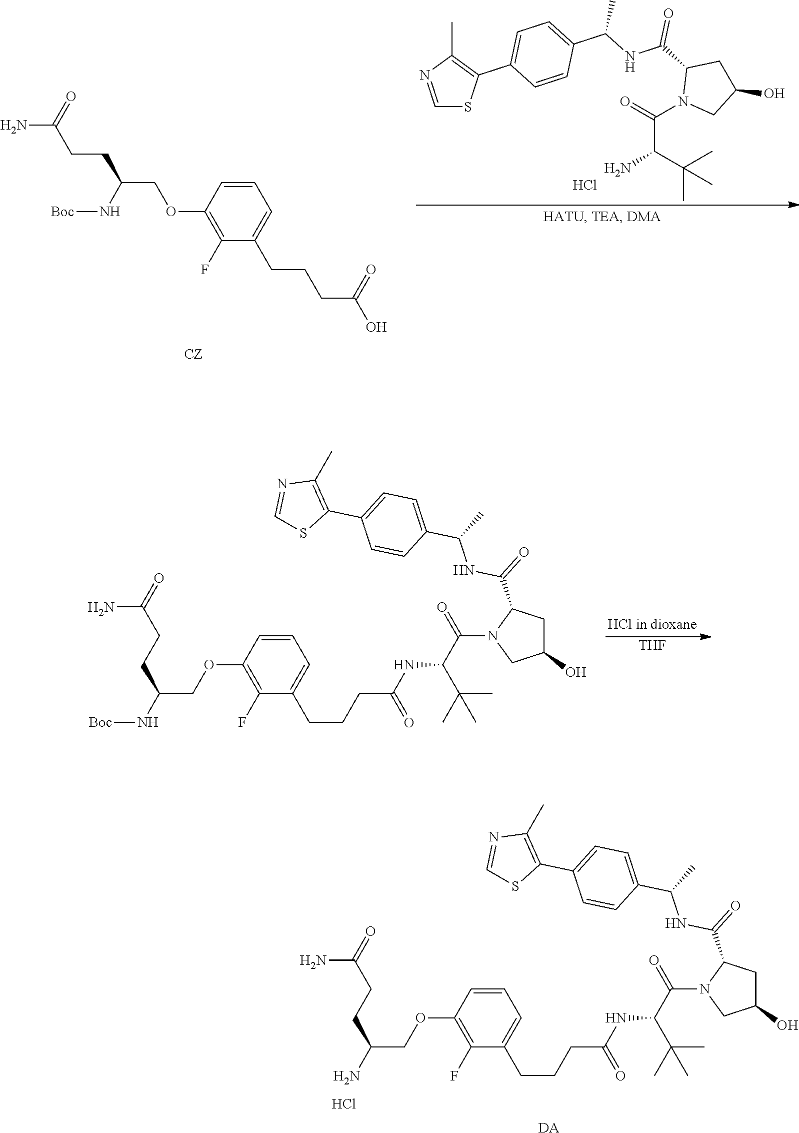

Step 1—Tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate. To a stirred mixture of 4-[3-[(2S)-2-[(tert-butoxycarbonyl)amino]-4-carbamoylbutoxy]-2-fluorophenyl]butanoic acid (250.00 mg, 0.606 mmol, Intermediate CZ) and (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (296.42 mg, 0.667 mmol) in DMA (6.00 mL) were added HATU (299.61 mg, 0.788 mmol) and TEA (245.33 mg, 2.424 mmol) at rt under nitrogen atmosphere. The resulting mixture was stirred for 16 h at rt under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 33%-60% B in 25 min; Flow rate: 80 mL/min; Detector: 220/254 nm; desired fractions were collected at 55% B) and concentrated under reduced pressure to afford the title compound (350 mg, 69% yield) as a off-white solid. ¹H NMR (300 MHz, Chloroform-d) δ 8.69 (s, 1H), 7.50 (d, J=7.7 Hz, 1H), 7.46-7.36 (m, 5H), 7.28 (s, 1H), 7.01-6.93 (m, 1H), 6.86-6.75 (m, 2H), 6.51-6.42 (m, 1H), 6.34-6.21 (m, 1H), 5.77 (s, 1H), 5.23-5.02 (m, 2H), 4.76-4.69 (m, 1H), 4.62-4.49 (m, 2H), 4.17-3.92 (m, 5H), 3.67-3.60 (m, 1H), 2.74-2.61 (m, 1H), 2.54 (s, 3H), 2.52-2.46 (m, 1H), 2.40-2.32 (m, 2H), 2.27-2.19 (m, 2H), 2.16-1.87 (m, 4H), 1.49 (d, J=7.3 Hz, 3H), 1.46 (s, 9H), 1.06 (s, 9H); LC/MS (ESI, m/z): [(M+H)]+=839.4.

Step 2—(2S,4R)-1-[(2S)-2-(4-[3-[(2S)-2-amino-4-carbamoylbutoxy]-2-fluorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-4-carbamoyl-1-[2-fluoro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamate (320.00 mg) in THF (5.00 mL) was added a solution of 4 M HCl (gas) in 1,4-dioxane (2.00 mL) dropwise at rt. The resulting mixture was stirred for 2 h at rt. On completion, the reaction mixture was concentrated under reduced pressure to afford the title compound (280 mg) as a off-white solid. ¹H NMR (400 MHz, DMSO-d6) δ 9.03 (s, 1H), 8.47-8.24 (m, 4H), 7.87 (d, J=9.2 Hz, 1H), 7.49-7.41 (m, 3H), 7.40-7.37 (m, 2H), 7.10-7.02 (m, 2H), 6.97-6.85 (m, 2H), 4.96-4.88 (m, 1H), 4.55-4.50 (m, 1H), 4.46-4.40 (m, 1H), 4.31-4.03 (m, 5H), 3.70-3.49 (m, 2H), 2.63-2.56 (m, 1H), 2.46 (s, 3H), 2.36-2.25 (m, 3H), 2.22-2.13 (m, 1H), 2.05-1.99 (m, 1H), 1.97-1.86 (m, 2H), 1.85-1.69 (m, 4H), 1.38 (d, J=7.0 Hz, 3H), 0.95 (s, 9H); LC/MS (ESI, m/z): [(M+H)]+=739.4.

### (2S)-2-amino-N-[1-(4-methanesulfonylphenyl)cyclopropyl]pentanediamide (Intermediate DB)

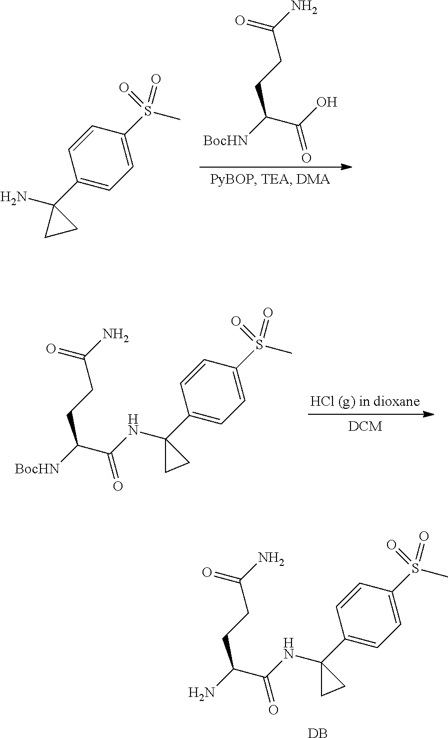

Step 1—Tert-butyl N-[(1S)-3-carbamoyl-1-[[1-(4-methanesulfonylphenyl)cyclopropyl]carbamoyl]propyl]carbamate. To a stirred mixture of 1-(4-methanesulfonylphenyl)cyclopropan-1-amine (240.00 mg, 1.14 mmol, CAS #1038389-00-2) and (2S)-2-[(tert-butoxycarbonyl)amino]-4-carbamoylbutanoic acid (279.74 mg, 1.14 mmol) in DMA (3.00 mL) were added PyBOP (768.47 mg, 1.48 mmol) and TEA (0.474 mL, 3.41 mmol) in turns at rt under nitrogen atmosphere. The resulting mixture was stirred for 1 h at rt under nitrogen atmosphere. On completion, the reaction was purified directly by reverse phase flash chromatography (Column: Spherical C18, 20-40 um, 330 g; Mobile Phase A: Water (plus 10 mmol/L FA); Mobile Phase B: ACN; Flow rate: 80 mL/min; Gradient: 30% B-45% B gradient in 20 min; Detector: 220 nm; the fractions containing the desired product were collected at 41% B) and concentrated under reduced pressure to afford the title compound (427 mg, 86% yield) as a white solid. ¹H NMR (300 MHz, DMSO-d6) δ 8.73 (s, 1H), 7.78-7.74 (m, 2H), 7.40-7.36 (m, 2H), 7.29 (s, 1H), 6.99-6.95 (m, 1H), 6.78 (s, 1H), 3.88-3.84 (m, 1H), 3.18 (s, 3H), 2.13-2.07 (m, 2H), 1.87-1.70 (m, 2H), 1.43 (s, 9H), 1.36-1.22 (m, 4H); LC/MS (ESI, m/z): [(M+H)]+=440.1.

Step 2—(2S)-2-amino-N-[1-(4-methanesulfonylphenyl)cyclopropyl]pentanediamide. To a stirred solution of tert-butyl N-[(1S)-3-carbamoyl-1-[[1-(4-methanesulfonylphenyl)cyclopropyl]carbamoyl]propyl]carbamate (427.00 mg, 0.971 mmol) in DCM (10.00 mL) was added HCl (gas) in 1,4-dioxane (5.00 mL) in portions at 0° C. under nitrogen atmosphere. The resulting mixture was stirred for 40 min at rt under nitrogen atmosphere. On completion, the reaction mixture was concentrated under reduced pressure to afford the title compound (420 mg) as a white solid. ¹H NMR (400 MHz, DMSO-d6) δ 9.52 (s, 1H), 8.43-8.39 (m, 2H), 7.86-7.78 (m, 2H), 7.53 (s, 1H), 7.50-7.41 (m, 2H), 6.95 (s, 1H), 3.88-3.84 (m, 1H), 3.18 (s, 3H), 2.22-2.18 (m, 2H), 2.02-2.00 (m, 2H), 1.34-1.28 (m, 4H); LC/MS (ESI, m/z): [(M+H)]+=340.2.

### 3-(1-aminocyclopropyl)propenamide (Intermediate DB)

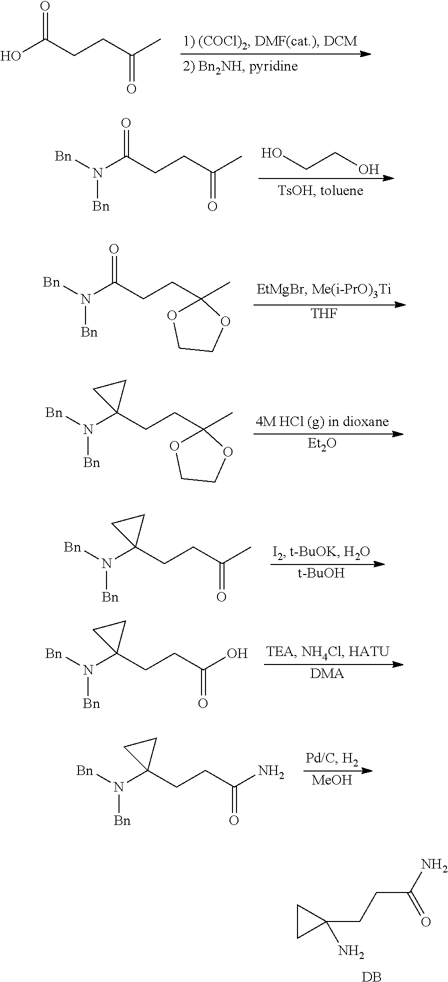

Step 1—N,N-dibenzyl-4-oxopentanamide. To a stirred solution of levulinic acid (5.00 g, 43.06 mmol) and (COCl)₂ (8.20 g, 64.59 mmol) in DCM (100.00 mL) was added DMF (31.47 mg, 0.431 mmol) dropwise at 0° C. under nitrogen atmosphere. The resulting mixture was stirred for 1 h at rt under nitrogen atmosphere. On completion, the reaction mixture was concentrated under vacuum. The residue was then re-dissolved in Et₂O (100.00 mL). To the solution was added pyridine (4.09 g, 51.67 mmol) and dibenzylamine (9.34 g, 47.37 mmol) at 0° C. The resulting mixture was then stirred overnight at rt. On completion, the reaction mixture was extracted with EtOAc (3×50 mL). The combined organic layers were washed with brine (1×30 mL), and dried over anhydrous Na2SO4. After filtration, the filtrate was concentrated under reduced pressure to give the title compound (9.6 g) as a brown yellow solid. ¹H NMR (300 MHz, Chloroform-d) δ 7.42-7.19 (m, 10H), 4.66-4.58 (m, 2H), 4.56-4.48 (m, 2H), 2.92-2.82 (m, 2H), 2.79-2.67 (m, 2H), 2.26 (s, 3H); LC/MS (ESI, m/z): [(M+H)]+=296.2.

Step 2—N,N-dibenzyl-3-(2-methyl-1,3-dioxolan-2-yl)propenamide. To a stirred solution of N,N-dibenzyl-4-oxopentanamide (9.60 g, 0.033 mmol) and ethylene glycol (3.03 g, 0.049 mmol) in toluene (100.00 mL) was added TsOH (0.56 g, 0.003 mmol) in portions at 25° C. under nitrogen atmosphere. The resulting mixture was stirred reflux for 16 h under nitrogen atmosphere. On completion, the reaction mixture was concentrated under reduced pressure. The crude product was purified by reverse phase flash (Column: Spherical C18 Column, 20-40 um, 330 g; Mobile Phase A: Water (5 mmol/L NH₄HCO₃), Mobile Phase B: ACN; Flow rate: 100 mL/min; Gradient: 40% B to 60% B in 25 min, 254 nm; the fractions containing the desired product were collected at 52% B) to afford the title compound (8.9 g, 80.68%) as a colorless oil; 1H NMR (300 MHz, DMSO-d6) δ 7.42-7.19 (m, 10H), 4.56-4.50 (m, 4H), 3.86-3.80 (m, 2H), 3.80-3.74 (m, 2H), 2.45-2.38 (m, 2H), 1.93-1.85 (m, 2H), 1.20 (s, 3H); LC/MS (ESI, m/z): [(M+H)]+=340.1.

Step 3—N,N-dibenzyl-1-[2-(2-methyl-1,3-dioxolan-2-yl)ethyl]cyclopropan-1-amine. To a stirred solution of N,N-dibenzyl-3-(2-methyl-1,3-dioxolan-2-yl)propanamide (8.9 g, 26.22 mmol) and methyltris(propan-2-yloxy)titanium (31.40 mL, 31.40 mmol) in THF (100.00 mL) was added ethylmagnesium bromide (17.4 mL, 52.45 mmol) dropwise at 0° C. under nitrogen atmosphere. The resulting mixture was stirred for 16 h at 25° C. under nitrogen atmosphere. On completion, the reaction was quenched with water (20 mL) at 0° C. and the resulting mixture was filtered. The filter cake was washed with EtOAc (3×50 mL), and the filtrate was concentrated under reduced pressure. The residue product was purified by reverse phase flash (Column: Spherical C18 Column, 20-40 um, 330 g; Mobile Phase A: Water (5 mmol/L NH₄HCO₃), Mobile Phase B: ACN; Flow rate: 60 mL/min; Gradient: 80% B to 95% B in 25 min, 254 nm; the fractions containing the desired product were collected at 95% B) to afford the title compound (5.8 g, 63% yield) as a yellow oil. ¹H NMR (300 MHz, Chloroform-d) δ 7.25-7.15 (m, 10H), 3.99-3.92 (m, 4H), 3.81-3.75 (m, 4H), 1.81-1.72 (m, 2H), 1.71-1.63 (m, 2H), 1.36 (s, 3H), 0.48-0.43 (m, 2H), 0.32-0.27 (m, 2H); LC/MS (ESI, m/z): [(M+H)]+=352.1.

Step 4—4-[1-(dibenzylamino)cyclopropyl]butan-2-one. To a stirred solution of N,N-dibenzyl-1-[2-(2-methyl-1,3-dioxolan-2-yl)ethyl]cyclopropan-1-amine (5.80 g, 16.50 mmol) in Et2O (50.00 mL) was added 4 M HCl (gas) in 1,4-dioxane (50.00 mL) dropwise at 25° C. under nitrogen atmosphere. The resulting mixture was stirred for 2 h at 25° C. under nitrogen atmosphere. On completion, the reaction mixture was concentrated under vacuum. The residue was neutralized pH to 7-8 with saturated Na₂CO₃ (aq.) and the mixture was extracted with Et2O (2×100 mL). The combined organic layers were washed with NaHCO₃ (aq.) (2×100 mL), and dried over anhydrous Na₂SO₄. After filtration, the filtrate was concentrated under reduced pressure. The crude product was purified by reverse phase flash (Column: Spherical C18 Column, 20-40 um, 330 g; Mobile Phase A: Water (5 mmol/L NH₄HCO₃), Mobile Phase B: ACN; Flow rate: 60 mL/min; Gradient: 80% B to 95% B in 25 min, 254 nm; the fractions containing the desired product were collected at 90% B) to afford the title compound (4.5 g, 89%) as a brown oil. ¹H NMR (300 MHz, Chloroform-d) δ 7.25-7.15 (m, 10H), 3.76-3.73 (m, 4H), 2.49-2.42 (m, 2H), 2.14 (s, 3H), 1.97-1.89 (m, 2H), 0.53-0.48 (m, 2H), 0.30-0.25 (m, 2H); LC/MS (ESI, m/z): [(M+H)]+=308.1.

Step 5—3-[1-(dibenzylamino)cyclopropyl]propanoic acid. To a stirred solution of t-BuOK (2.92 g, 26.022 mmol) in t-BuOH (40.00 mL) was added 12 (2.48 g, 9.76 mmol) at 25° C. under nitrogen atmosphere. The resulting mixture was stirred for 30 min at 10° C. under nitrogen atmosphere. To the above mixture was added H₂O (0.18 g, 9.99 mmol) dropwise at 25° C. To the reaction mixture was added 4-[1-(dibenzylamino)cyclopropyl]butan-2-one (1.00 g, 3.25 mmol) in t-BuOH (10.00 mL) dropwise over 10 min at 25° C. The resulting mixture was stirred for additional 16 h at 25° C. On completion, the reaction mixture was filtered, and the filter cake was washed with EtOAc (3×50 mL). The filtrate was concentrated under reduced pressure. The crude product was purified by reverse phase flash (Column: Spherical C18 Column, 20-40 um, 330 g; Mobile Phase A: Water (5 mmol/L NH₄HCO₃), Mobile Phase B: ACN; Flow rate: 100 mL/min; Gradient: 30% B to 50% B in 25 min, 254 nm; the fractions containing the desired product were collected at 40% B) to afford the title compound (730 mg, 73% yield) as a yellow solid. ¹H NMR (300 MHz, Chloroform-d) δ 7.29-7.20 (m, 10H), 3.78-3.74 (m, 4H), 2.50-2.42 (m, 2H), 1.97-1.93 (m, 2H), 0.67-0.62 (m, 2H), 0.40-0.35 (m, 2H); LC/MS (ESI, m/z): [(M+H)]+=310.1.

Step 6—3-[1-(dibenzylamino)cyclopropyl]propenamide. To a stirred solution of 3-[1-(dibenzylamino)cyclopropyl]propanoic acid (590.00 mg, 1.91 mmol) and NH₄Cl (202 mg, 3.82 mmol) in DMA (5.00 mL) were added TEA (0.795 mL, 5.73 mmol) and HATU (1.088 g, 2.87 mmol) in turns at 25° C. under nitrogen atmosphere. The resulting mixture was stirred for 2 h at 25° C. under nitrogen atmosphere. On completion, the reaction mixture was concentrated under reduced pressure. The crude product was purified by reverse phase flash (Column: Spherical C18 Column, 20-40 um, 120 g; Mobile Phase A: Water (5 mmol/L NH₄HCO₃), Mobile Phase B: ACN; Flow rate: 60 mL/min; Gradient: 50% B to 80% B in 25 min, 254 nm; the fractions containing the desired product were collected at 73% B) to afford the title compound (470 mg, 80%) as a white solid. ¹H NMR (400 MHz, DMSO-d6) δ 7.26-7.14 (m, 10H), 6.75 (broad, 2H), 3.73-3.68 (m, 4H), 2.12-2.06 (m, 2H), 1.93-1.86 (m, 2H), 0.39-0.32 (m, 2H), 0.31-0.26 (m, 2H); LC/MS (ESI, m/z): [(M+H)]+=309.1.

Step 7—3-(1-aminocyclopropyl)propenamide. To a stirred solution of 3-[1-(dibenzylamino)cyclopropyl]propanamide (230.00 mg, 0.746 mmol) in MeOH (7.00 mL) was added Pd/C (100.00 mg) at 25° C. under nitrogen atmosphere. The resulting mixture was stirred for 2 h at 25° C. under hydrogen atmosphere. On completion, the reaction mixture was filtered and the filter cake was washed with EtOAc (3×30 mL). The filtrate was concentrated under reduced pressure to give the title compound (90 mg) as an off-white solid. ¹H NMR (300 MHz, Methanol-d4) δ 2.42-2.34 (m, 2H), 1.78-1.70 (m, 2H), 0.60-0.53 (m, 2H), 0.52-0.44 (m, 2H); LC/MS (ESI, m/z): [(M+H)]+=129.1.

### (2S)-1-[(2S)-2-[(tert-butoxycarbonyl)amino]propanoyl]pyrrolidine-2-carboxylic acid (Intermediate DC)

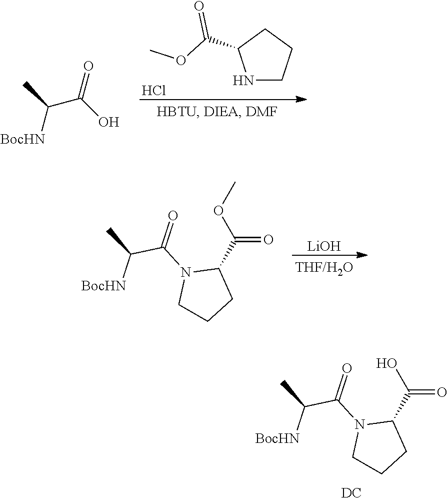

Step 1—Methyl (2S)-1-[(2S)-2-[(tert-butoxycarbonyl)amino]propanoyl]pyrrolidine-2-carboxylate. To a stirred solution of (2S)-2-[(tert-butoxycarbonyl)amino]propanoic acid (5.00 g, 26.43 mmol) and methyl (2S)-pyrrolidine-2-carboxylate hydrochloride (5.69 g, 34.35 mmol) in DMF (50.00 mL) were added DIEA (13.66 g, 105.7 mmol) and HBTU (13.03 g, 34.35 mmol) in turns at rt under nitrogen atmosphere. The resulting mixture was stirred for 2 h at rt under nitrogen atmosphere. On completion, the reaction mixture was diluted with water (400 mL) and the mixture was extracted with EtOAc (5×100 mL). The combined organic layers were washed with brine (5×250 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (10 mmol/L NH₄HCO₃); Eluent B: ACN; Gradient: 20%-40% B in 25 min; Flow rate: 80 mL/min; Detector: 220/254 nm; desired fractions were collected at 35% B) and concentrated under reduced pressure to afford the title compound (7.38 g, 93% yield) as a yellow oil. ¹H NMR (300 MHz, DMSO-d6) δ 6.95 (d, J=7.5 Hz, 1H), 4.36-4.32 (m, 1H), 4.29-4.25 (m, 1H), 3.68-3.64 (m, 1H), 3.62 (s, 3H), 3.58-3.52 (m, 1H), 2.28-2.11 (m, 1H), 1.97-1.93 (m, 2H), 1.85-1.81 (m, 1H), 1.38 (s, 9H), 1.19-1.13 (m, 3H); LC/MS (ESI, m/z): [(M+H)]+=301.1.

Step 2—(2S)-1-[(2S)-2-[(tert-butoxycarbonyl)amino]propanoyl]pyrrolidine-2-carboxylic acid. To a stirred solution of methyl (2S)-1-[(2S)-2-[(tert-butoxycarbonyl)amino]propanoyl]pyrrolidine-2-carboxylate (7.30 g, 24.31 mmol,) in THF (70.00 mL) were added LiOH (4.66 g, 194.44 mmol) and H₂O (70.00 mL) in turns at rt under nitrogen atmosphere. The resulting mixture was stirred for 2 h at rt under nitrogen atmosphere. On completion, the reaction was concentrated under reduced pressure. The residue was purified by reverse phase flash chromatography (Column: WelFlash™ C18-I, 20-40 um, 330 g; EluentA: Water (10 mmol/L NH₄HCO₃); Eluent B: ACN; Gradient: 10%-30% B in 25 min; Flow rate: 80 mL/min; Detector: 220/254 nm; desired fractions were collected at 28% B) and concentrated under reduced pressure to afford the title compound (5.41 g, 78% yield) as a white solid. ¹H NMR (300 MHz, DMSO-d6) δ 11.75 (s, 1H), 6.94 (d, J=7.5 Hz, 1H), 4.27-4.23 (m, 2H), 3.65-3.61 (m, 1H), 3.54-3.50 (m, 1H), 2.22-2.07 (m, 1H), 2.04-1.76 (m, 3H), 1.38 (s, 9H), 1.25-1.10 (m, 3H); LC/MS (ESI, m/z): [(M+H)]+=287.3.

### (2S)-1-[(2S)-2-[(tert-butoxycarbonyl)amino]-2-cyclohexylacetyl]pyrrolidine-2-carboxylic acid (Intermediate DD)

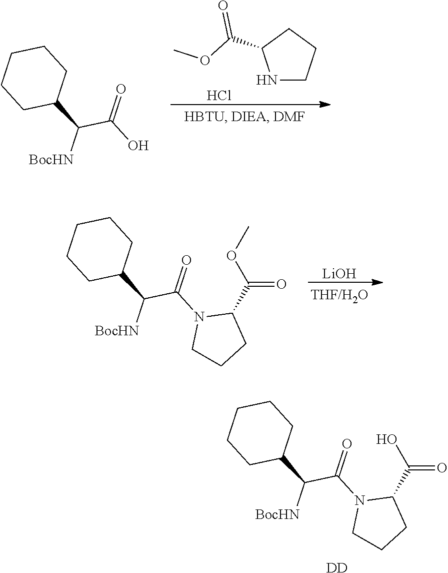

Step 1—Methyl (2S)-1-[(2S)-2-[(tert-butoxycarbonyl)amino]-2-cyclohexylacetyl]pyrrolidine-2-carboxylate. To a solution of (S)-[(tert-butoxycarbonyl)amino](cyclohexyl)acetic acid (5.00 g, 19.43 mmol) and methyl (2S)-pyrrolidine-2-carboxylate hydrochloride (4.18 g, 25.26 mmol) in DMF (50.00 mL) were added DIEA (12.8 mL, 77.72 mmol) dropwise and HBTU (9.58 g, 25.26 mmol) in portions at rt. The resulting mixture was stirred for 16 h at rt. On completion, the water (100 mL) was added to the mixture which was then extracted with EtOAc (5×100 mL). The combined organic layers were washed with brine (5×100 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; EluentA: Water (plus 10 mmol/L NH₄HCO₃); Eluent B: ACN; Gradient: 35%-55% B in 25 min; Flow rate: 80 mL/min; Detector: 220/254 nm; desired fractions were collected at 51% B) and concentrated under reduced pressure to afford the title compound (6.57 g, 92% yield) as a yellow oil. ¹H NMR (300 MHz, DMSO-d6) δ 6.87-6.76 (m, 1H), 4.37-4.28 (m, 1H), 4.07 (t, J=8.4 Hz, 1H), 3.90-3.72 (m, 1H), 3.62 (s, 3H), 3.59-3.51 (m, 1H), 2.28-2.11 (m, 1H), 2.00-1.80 (m, 2H), 1.80-1.55 (m, 6H), 1.38 (s, 9H), 1.13 (d, J=8.0 Hz, 4H), 1.05-0.87 (m, 2H); LC/MS (ESI, m/z): [(M+H)]+=369.2.

Step 2—(2S)-1-[(2S)-2-[(tert-butoxycarbonyl)amino]-2-cyclohexylacetyl]pyrrolidine-2-carboxylic acid. To a stirred mixture of methyl (2S)-1-[(2S)-2-[(tert-butoxycarbonyl)amino]-2-cyclohexylacetyl]pyrrolidine-2-carboxylate (6.59 g, 17.89 mmol) in THF (70.00 mL) was added LiOH (3.43 g, 143.08 mmol) in H₂O (70.00 mL) dropwise at rt. The resulting mixture was stirred for 2 h at rt. On completion, the reaction mixture was concentrated under reduced pressure. The residue was purified by reverse phase flash chromatography (Column: WelFlash™ C18-I, 20-40 um, 330 g; EluentA: Water (plus 10 mmol/L FA); Eluent B: ACN; Gradient: 15%-35% B in 25 min; Flow rate: 80 mL/min; Detector: 220/254 nm; desired fractions were collected at 21% B) and concentrated under reduced pressure to afford the title compound (3.76 g, 59% yield) as a white solid. ¹H NMR (300 MHz, DMSO-d6) δ 12.75-11.65 (m, 1H), 6.76 (d, J=8.5 Hz, 1H), 4.30-4.19 (m, 1H), 4.12-3.98 (m, 1H), 3.83-3.69 (m, 1H), 3.63-3.52 (m, 1H), 2.23-2.08 (m, 1H), 2.03-1.84 (m, 2H), 1.85-1.74 (m, 2H), 1.72-1.56 (m, 4H), 1.38 (s, 9H), 1.25-1.08 (m, 4H), 1.04-0.90 (m, 2H); LC/MS (ESI, m/z): [(M+H)]+=355.2.

### Benzyl (1S,4S)-4-(((1-(2,6-dioxopiperidin-3-yl)-3-methyl-2-oxo-2,3-dihydro-1H-benzo[d]imidazol-5-yl)amino)methyl)cyclohexane-1-carboxylate (Intermediate DE) and Benzyl (1R,4R)-4-(((1-(2,6-dioxopiperidin-3-yl)-3-methyl-2-oxo-2,3-dihydro-1H-benzo[d]imidazol-5-yl)amino)methyl)cyclohexane-1-carboxylate (Intermediate DF)

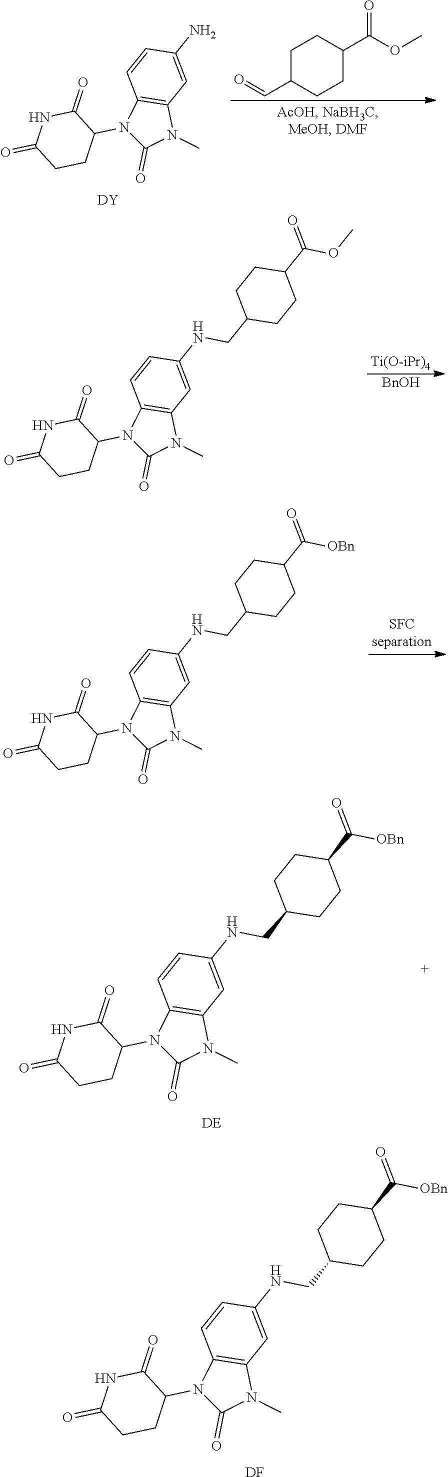

Step 1—Methyl 4-([[1-(2,6-dioxopiperidin-3-yl)-3-methyl-2-oxo-1,3-benzodiazol-5-yl]amino]methyl)cyclohexane-1-carboxylate. To a stirred mixture of 3-(5-amino-3-methyl-2-oxo-1,3-benzodiazol-1-yl)piperidine-2,6-dione (50.00 mg, 0.182 mmol, Intermediate DY) and methyl 4-formylcyclohexane-1-carboxylate (34.13 mg, 0.201 mmol) in MeOH (1.00 mL) and DMF (1.00 mL) were added NaBH₃CN (22.91 mg, 0.365 mmol) and AcOH (54.74 mg, 0.912 mmol) in portions at rt. The resulting mixture was stirred for 4 h at rt under nitrogen atmosphere. On completion, the reaction mixture was concentrated under vacuum. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 35%-65% B in 25 min; Flow rate: 80 mL/min; Detector: 220/254 nm; desired fractions were collected at 51% B) and concentrated under reduced pressure to afford the title compound (30 mg, 61% yield) as a white solid. ¹H NMR (300 MHz, DMSO-d6) δ 11.05 (s, 1H), 6.83-6.75 (m, 1H), 6.42-6.36 (m, 1H), 6.32-6.26 (m, 1H), 5.78-5.36 (m, 1H), 5.24 (dd, J=12.4, 5.3 Hz, 1H), 3.31 (s, 3H), 2.87 (s, 3H), 2.85-2.76 (m, 2H), 2.70-2.58 (m, 2H), 2.37-2.19 (m, 1H), 2.03-1.89 (m, 5H), 1.68-1.53 (m, 2H), 1.39-1.16 (m, 2H), 1.08-0.96 (m, 1H); LC/MS (ESI, m/z): [(M+H)]+=429.2.

Step 2—Benzyl 4-([[1-(2,6-dioxopiperidin-3-yl)-3-methyl-2-oxo-1,3-benzodiazol-5-yl]amino]methyl)cyclohexane-1-carboxylate. To a stirred solution of methyl 4-([[1-(2,6-dioxopiperidin-3-yl)-3-methyl-2-oxo-1,3-benzodiazol-5-yl]amino]methyl)cyclohexane-1-carboxylate (50.00 mg, 0.117 mmol) in benzyl alcohol (1.00 mL) was added Ti(Oi-Pr)₄ (8.29 mg, 0.029 mmol) in portions at rt under nitrogen atmosphere. The resulting mixture was stirred for 1 h at 120° C. under nitrogen atmosphere. On completion, the reaction mixture was allowed to cool down to rt. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 30%-80% B in 25 min; Flow rate: 80 mL/min; Detector: 220/254 nm; desired fractions were collected at 67% B) and concentrated under reduced pressure to afford the title compound (650 mg, 58% yield) as a white solid. ¹H NMR (300 MHz, DMSO-d6) δ 11.04 (s, 1H), 7.72-7.28 (m, 5H), 6.79 (d, J=8.3 Hz, 1H), 6.39 (s, 1H), 6.29 (d, J=8.2 Hz, 1H), 5.39-5.32 (m, 1H), 5.27 (dd, J=12.4, 5.3 Hz, 1H), 5.11 (s, 2H), 3.28 (s, 3H), 2.85-2.76 (m, 2H), 2.70-2.58 (m, 2H), 2.37-2.19 (m, 1H), 2.03-1.89 (m, 5H), 1.68-1.53 (m, 2H), 1.39-1.16 (m, 2H), 1.08-0.96 (m, 1H); LC/MS (ESI, m/z): [(M+H)]+=505.2.

Step 3—Benzyl (1S,4S)-4-(((1-(2,6-dioxopiperidin-3-yl)-3-methyl-2-oxo-2,3-dihydro-1H-benzo[d]imidazol-5-yl)amino)methyl)cyclohexane-1-carboxylate and Benzyl (1R,4R)-4-(((1-(2,6-dioxopiperidin-3-yl)-3-methyl-2-oxo-2,3-dihydro-1H-benzo[d]imidazol-5-yl)amino)methyl)cyclohexane-1-carboxylate. The mixture benzyl 4-([[1-(2,6-dioxopiperidin-3-yl)-3-methyl-2-oxo-1,3-benzodiazol-5-yl]amino]methyl)cyclohexane-1-carboxylate (750 mg) was separated by SFC with the following conditions: Column: CHIRAL ART Cellulose-SB, 3*25 cm (5 um); Mobile Phase A: MTBE (0.1% DEA), Mobile Phase B: MeOH; Flow rate: 45 mL/min; Gradient: 15 B to 15 B in 20 min; 220/254 nm; RT1: 13.1; RT2: 14.3; RT3: 15.5. Absolute stereochemistry for the diastereomers were assigned arbitrarily. The first peak fractions (RT1: 13.1 min) were collected, roto-evaporated in vacuo to give 320 mg (R configuration, aribitrarly assigned). The second peak fractions (RT2: 14.3 min) were collected, roto-evaporated in vacuo to give 100 mg (S configuration and Cis isomer, aribitrarly assigned). The third peak fractions (RT3: 15.5 min) were collected, roto-evaporated in vacuo to give 210 mg (S configuration and Trans isomer, aribitrarly assigned).

The 320 mg (R configuration, assumed) was further separated by SFC with the following conditions: Column: CHIRALPAK IA, 3*25 cm (5 um); Mobile Phase A: MTBE, Mobile Phase B: EtOH; Flow rate: 45 mL/min; Gradient: 50 B to 50 B in 30 min; 220/254 nm; RT1:9.2; RT2:17.2.

The first peak fractions (RT11: 9.2 min) were collected, roto-evaporated in vacuo to give 80 mg (R configuration and Cis isomer, aribitrarly assigned). The second peak fractions (RT12: 17.2 min) were collected, roto-evaporated in vacuo to give 170 mg (R configuration and Trans isomer, aribitrarly assigned).

The first peaks fractions (RT2: 14.3 min and RT11: 9.2 min) were collected, roto-evaporated in vacuo to give benzyl (1s,4s)-4-([[1-(2,6-dioxopiperidin-3-yl)-3-methyl-2-oxo-1,3-benzodiazol-5-yl]amino]methyl)cyclohexane-1-carboxylate (180 mg) as a white solid. 1H NMR (300 MHz, DMSO-d6) δ 11.04 (s, 1H), 7.43-7.38 (m, 3H), 7.36-7.28 (m, 2H), 6.81 (d, J=8.4 Hz, 1H), 6.42 (s, 1H), 6.31 (d, J=8.4 Hz, 1H), 5.39-5.32 (m, 1H), 5.27 (dd, J=12.4, 5.3 Hz, 1H), 5.11 (s, 2H), 3.28 (s, 3H), 2.92-2.82 (m, 2H), 2.71-2.59 (m, 3H), 2.02-1.87 (m, 3H), 1.71-2.52 (m, 5H), 1.29-1.18 (m, 2H); LC/MS (ESI, m/z): [(M+1)]+=505.2.

The second peaks fractions (RT3: 15.5 min and RT12: 17.2 min) were collected, roto-evaporated in vacuo to give benzyl (1r,4r)-4-([[1-(2,6-dioxopiperidin-3-yl)-3-methyl-2-oxo-1,3-benzodiazol-5-yl]amino]methyl)cyclohexane-1-carboxylate (380 mg) as a white solid. 1H NMR (300 MHz, DMSO-d6) δ 11.04 (s, 1H), 7.43-7.38 (m, 3H), 7.36-7.28 (m, 2H), 6.81 (d, J=8.4 Hz, 1H), 6.42 (s, 1H), 6.31 (d, J=8.4 Hz, 1H), 5.39-5.32 (m, 1H), 5.27 (dd, J=12.4, 5.3 Hz, 1H), 5.11 (s, 2H), 3.28 (s, 3H),) 2.92-2.82 (m, 2H), 2.68-2.59 (m, 1H), 2.40-2.27 (m, 1H), 2.03-1.86 (m, 5H), 1.62-1.48 (m, 1H), 1.42-1.21 (m, 3H), 1.08-0.96 (m, 2H); LC/MS (ESI, m/z): [(M+1)]+=505.2.

### (1S,4S)-4-([[1-(2,6-dioxopiperidin-3-yl)-3-methyl-2-oxo-1,3-benzodiazol-5-yl]amino]methyl)cyclohexane-1-carboxylic acid (Intermediate DG)

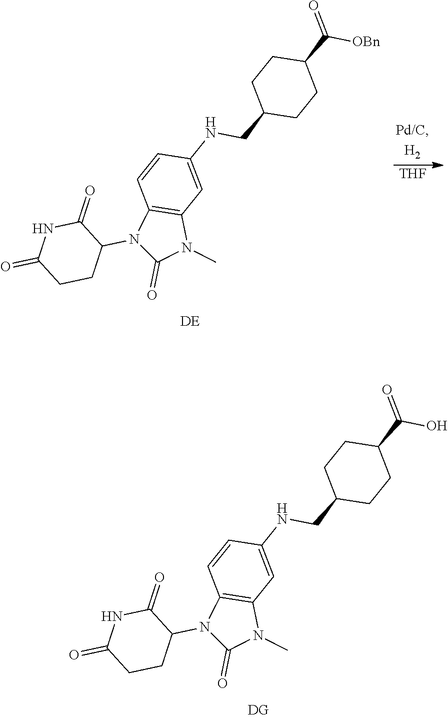 [SMILES: Cn1c(=O)n(C2CCC(=O)NC2=O)c2ccc(NC[C@H]3CC[C@@H](C(=O)OCc4ccccc4)CC3)cc21 ; Cn1c(=O)n(C2CCC(=O)NC2=O)c2ccc(NC[C@H]3CC[C@@H](C(=O)O)CC3)cc21 ; "THF"]

To a solution of benzyl (1S,Ss)-4-([[1-(2,6-dioxopiperidin-3-yl)-3-methyl-2-oxo-1,3-benzodiazol-5-yl]amino]methyl)cyclohexane-1-carboxylate (180.00 mg) in THF (10.00 mL) was added Pd/C (10 wt %, 20 mg) under nitrogen atmosphere in a 50 mL round-bottom flask. The mixture was hydrogenated at rt for 16 h under hydrogen atmosphere using a hydrogen balloon. On completion, the reaction was filtered through a celite pad and concentrated under reduced pressure to afford (140 mg, 95% yield) as a off-white solid. ¹H NMR (400 MHz, DMSO-d6) δ 12.00 (s, 1H), 11.04 (s, 1H), 6.79 (d, J=8.4 Hz, 1H), 6.39 (s, 1H), 6.28 (d, J=8.4, 1H), 5.35 (s, 1H), 5.23 (dd, J=12.8, 5.3 Hz, 1H), 3.26 (s, 3H), 2.92-2.82 (m, 2H), 2.73-2.56 (m, 2H), 2.47-2.42 (m, 1H), 1.97-1.93 (m, 1H), 1.69-1.58 (m, 3H), 1.48-1.40 (m, 2H), 1.25-1.20 (m, 4H); LC/MS (ESI, m/z): [(M+1)]+=415.2.

### (5S,8S,10aR)-5-[(tert-butoxycarbonyl)amino]-6-oxo-3-[(1s,4s)-4-([[1-(2,6-dioxopiperidin-3-yl)-3-methyl-2-oxo-1,3-benzodiazol- 5-yl]amino]methyl)cyclohexanecarbonyl]-octahydropyrrolo[1,2-a][1,5]diazocine-8-carboxylic acid (Intermediate DH)

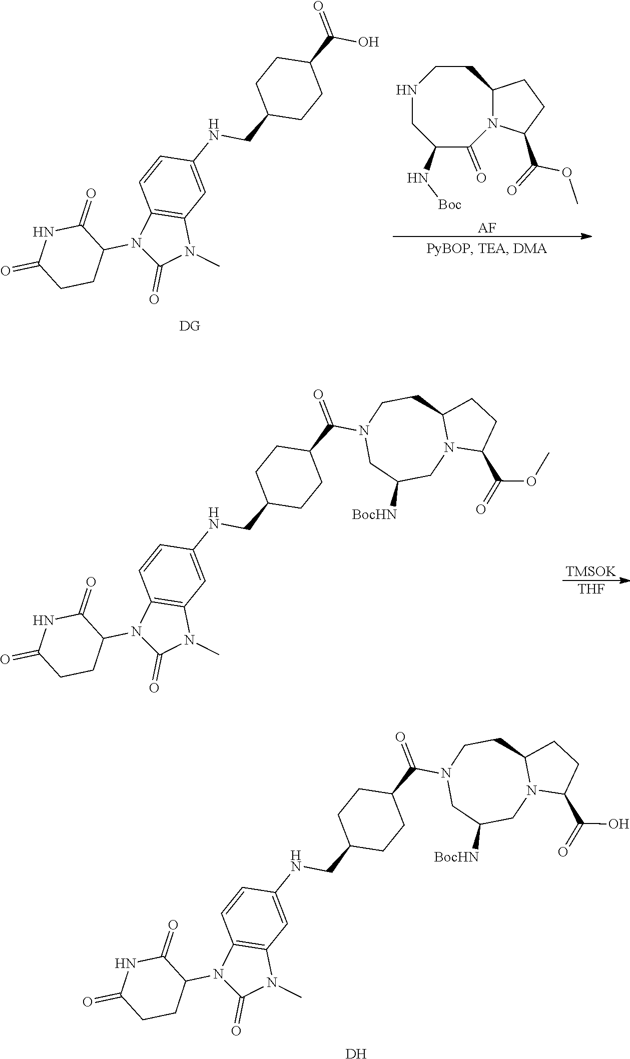

Step 1—Methyl (5S,8S,10aR)-5-[(tert-butoxycarbonyl)amino]-6-oxo-3-[(1s,4s)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylate. To a stirred mixture of (1s,4s)-4-([[1-(2,6-dioxopiperidin-3-yl)-3-methyl-2-oxo-1,3-benzodiazol-5-yl]amino]methyl)cyclohexane-1-carboxylic acid (140.00 mg, 0.338 mmol, Intermediate DG), PyBOP (228.52 mg, 0.439 mmol) and TEA (0.14 mL, 1.013 mmol) in DMA (4.00 mL) was added methyl (5S,8S,10aR)-5-[(tert-butoxycarbonyl)amino]-6-oxo-octahydro-1H-pyrrolo[1,2-a][1,5]diazocine-8-carboxylate (126.86 mg, 0.372 mmol, Intermediate AF) at rt under nitrogen atmosphere. The resulting mixture was stirred for 16 h at rt under nitrogen atmosphere. On completion, the residue was purified directly by reverse phase flash chromatography (Column: WelFlash™ C18-I, 20-40 um, 120 g; EluentA: Water (plus 10 mmol/L FA); Eluent B: ACN; Gradient: 30%-50% B in 25 min; Flow rate: 60 mL/min; Detector: 220/254 nm; desired fractions were collected at 43% B) and concentrated under reduced pressure to afford the title compound (180 mg, 72% yield) as a off-white solid. ¹H NMR (300 MHz, DMSO-d6) δ 11.04 (s, 1H), 8.15 (s, 1H), 6.80 (d, J=8.5 Hz, 1H), 6.70 (d, J=7.7 Hz, 1H), 6.39 (s, 1H), 6.29 (d, J=8.4 Hz, 1H), 5.23 (dd, J=12.8, 5.3 Hz, 1H), 4.61-4.53 (m, 1H), 4.34-4.29 (m, 1H), 4.21-4.16 (m, 1H), 3.62 (s, 3H), 3.26 (s, 3H), 3.03-0.90 (m, 4H), 2.87-2.75 (m, 3H), 2.70-2.55 (m, 3H), 2.29-2.23 (m, 1H), 1.94-1.87 (m, 1H), 1.82-1.67 (m, 7H), 1.42 (s, 9H), 1.29-1.22 (m, 3H), 1.07-0.92 (m, 4H); LC/MS (ESI, m/z): [(M+1)]+=738.4.

Step 2—(5S,8S,10aR)-5-[(tert-butoxycarbonyl)amino]-6-oxo-3-[(1s,4s)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylic acid. To a stirred solution of methyl (5S,8S,10aR)-5-[(tert-butoxycarbonyl)amino]-6-oxo-3-[(1s,4s)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylate (180.00 mg, 0.244 mmol) in THF (10.00 mL) was added potassium trimethylsilanolate (62.59 mg, 0.488 mmol) at rt under nitrogen atmosphere. The resulting mixture was stirred for 3 h at rt under nitrogen atmosphere. On completion, the reaction was concentrated under vacuum. Then the residue was purified by reverse phase flash chromatography (Column: WelFlash™ C18-I, 20-40 um, 80 g; EluentA: Water (plus 10 mmol/L FA); Eluent B: ACN; Gradient: 20%-45% B in 25 min; Flow rate: 50 mL/min; Detector: 220/254 nm; desired fractions were collected at 35% B) and concentrated under reduced pressure to afford the title compound (95 mg, 54% yield) as a off-white solid. ¹H NMR (300 MHz, DMSO-d6) δ 12.25 (s, 1H), 11.04 (s, 1H), 8.15 (s, 1H), 6.80 (d, J=8.5 Hz, 1H), 6.70 (d, J=7.7 Hz, 1H), 6.39 (s, 1H), 6.29 (d, J=8.4 Hz, 1H), 5.23 (dd, J=12.8, 5.3 Hz, 1H), 4.61-4.53 (m, 1H), 4.34-4.29 (m, 1H), 4.21-4.16 (m, 1H), 3.26 (s, 3H), 3.03-0.90 (m, 4H), 2.87-2.75 (m, 3H), 2.70-2.55 (m, 3H), 2.29-2.23 (m, 1H), 1.94-1.87 (m, 1H), 1.82-1.67 (m, 7H), 1.42 (s, 9H), 1.29-1.22 (m, 3H), 1.07-0.92 (m, 4H); LC/MS (ESI, m/z): [(M+1)]+=724.4.

### (1R,4R)-4-([[1-(2,6-dioxopiperidin-3-yl)-3-methyl-2-oxo-1,3-benzodiazol-5-yl]amino]methyl)cyclohexane-1-carboxylic acid (Intermediate DI)

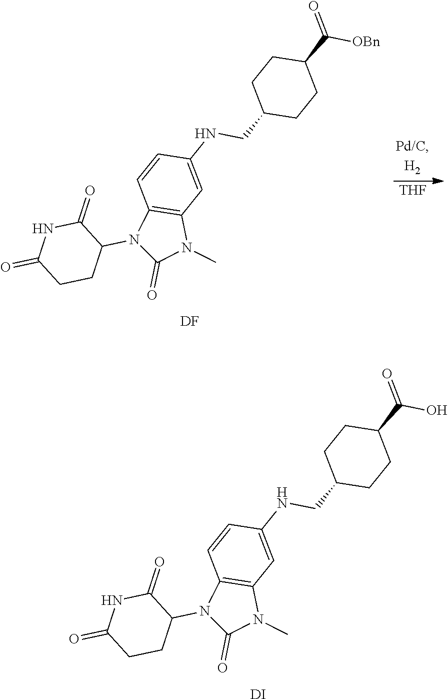

To a solution of benzyl (1R,4R)-4-([[1-(2,6-dioxopiperidin-3-yl)-3-methyl-2-oxo-1,3-benzodiazol-5-yl]amino]methyl)cyclohexane-1-carboxylate (380.00 mg, Intermediate DF) in THF (20.00 mL) was added Pd/C (10 wt %, 38 mg) under nitrogen atmosphere in a 50 mL round-bottom flask. The mixture was hydrogenated at rt for 16 h under hydrogen atmosphere using a hydrogen balloon. On completion, the reaction mixture was filtered through a celite pad and concentrated under reduced pressure to give the title compound (295 mg, 94% yield) as a off-white solid. ¹H NMR (400 MHz, DMSO-d6) δ 12.00 (s, 1H), 11.04 (s, 1H), 6.79 (d, J=8.4 Hz, 1H), 6.39 (s, 1H), 6.28 (d, J=8.4 Hz, 1H), 5.32 (s, 1H), 5.25 (dd, J=12.5, 5.4 Hz, 1H), 3.26 (s, 3H), 2.92-2.82 (m, 3H), 2.73-2.50 (m, 2H), 2.47-2.42 (m, 1H), 1.97-1.93 (m, 1H), 1.66-1.58 (m, 3H), 1.48-1.40 (m, 2H), 1.25-1.20 (m, 4H); LC/MS (ESI, m/z): [(M+1)]+=415.2.

### (5S,8S,10aR)-5-[(tert-butoxycarbonyl)amino]-6-oxo-3-[(1R,4R)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylic acid (Intermediate DJ)

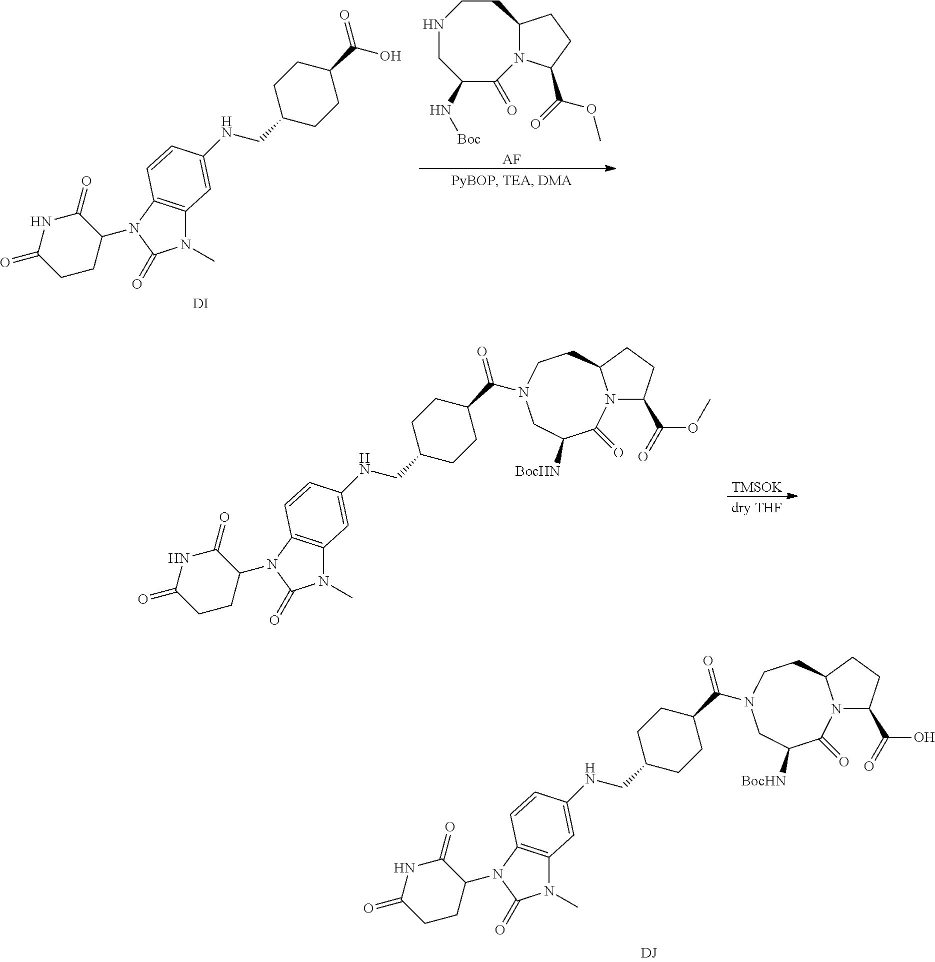

Step 1—Methyl (5S,8S,10aR)-5-[(tert-butoxycarbonyl)amino]-6-oxo-3-[(1r,4r)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylate. To a stirred mixture of (1R,4R)-4-([[1-(2,6-dioxopiperidin-3-yl)-3-methyl-2-oxo-1,3-benzodiazol-5-yl]amino]methyl)cyclohexane-1-carboxylic acid (295.00 mg, 0.712 mmol, Intermediate DI), PyBOP (481.52 mg, 0.925 mmol) and TEA (0.3 mL, 2.14 mmol,) in DMA (8.00 mL) was added methyl (5S,8S,10aR)-5-[(tert-butoxycarbonyl)amino]-6-oxo-octahydro-1H-pyrrolo[1,2-a][1,5]diazocine-8-carboxylate (267.30 mg, 0.783 mmol, Intermediate AF) at rt under nitrogen atmosphere. The resulting mixture was stirred for 16 h at rt under nitrogen atmosphere. On completion, the reaction mixture was purified directly by reverse phase flash chromatography (Column: WelFlash™ C18-I, 20-40 um, 330 g; Eluent A: Water (plus 10 mmol/L FA); Eluent B: ACN; Gradient: 33%-50% B in 25 min; Flow rate: 80 mL/min; Detector: 220/254 nm; desired fractions were collected at 44% B) and concentrated under reduced pressure to afford the title compound (350 mg, 67% yield) as a off-white solid. ¹H NMR (300 MHz, DMSO-d6) δ 11.04 (s, 1H), 8.15 (s, 1H), 6.80 (d, J=8.4 Hz, 1H), 6.70 (s, 1H), 6.39 (s, 1H), 6.29 (d, J=8.4 Hz, 1H), 5.23 (dd, J=12.5, 5.4 Hz, 1H), 4.57 (s, 1H), 4.34-4.29 (m, 1H), 4.23-4.15 (m, 2H), 3.62 (s, 3H), 3.28 (s, 3H), 3.03-2.90 (m, 4H), 2.87-2.75 (m, 3H), 2.70-2.55 (m, 3H), 2.28-2.21 (m 1H), 1.92-1.85 (m, 2H), 1.82-1.67 (m, 6H), 1.42 (s, 9H), 1.25 (d, J=3.2 Hz, 4H), 1.07-0.92 (m, 2H); LC/MS (ESI, m/z): [(M+1)]+=738.4.

Step 2—(5S,8S,10aR)-5-[(tert-butoxycarbonyl)amino]-6-oxo-3-[(1r,4r)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylic acid. To a stirred solution of methyl (5S,8S,10aR)-5-[(tert-butoxycarbonyl)amino]-6-oxo-3-[(1R,4R)-4-([[1-(2,6-dioxopiperidin-3-yl)-3-methyl-2-oxo-1,3-benzodiazol-5-yl]amino]methyl)cyclohexanecarbonyl]-octahydropyrrolo[1,2-a][1,5]diazocine-8-carboxylate (350.00 mg, 0.474 mmol) in THF (20.00 mL) was added potassium trimethylsilanolate (121.71 mg, 0.949 mmol) at rt under nitrogen atmosphere. The resulting mixture was stirred for 3 h at rt under nitrogen atmosphere. On completion, the reaction mixture was concentrated. Then the residue was purified by reverse phase flash chromatography (Column: WelFlash™ C18-I, 20-40 um, 330 g; Eluent A: Water (plus 10 mmol/L FA); Eluent B: ACN; Gradient: 20%-45% B in 25 min; Flow rate: 80 mL/min; Detector: 220/254 nm; desired fractions were collected at 36% B) and concentrated under reduced pressure to afford the title compound (233 mg, 68% yield) as a off-white solid. ¹H NMR (300 MHz, DMSO-d6) δ 11.04 (s, 1H), 8.15 (s, 1H), 6.80 (d, J=8.4 Hz, 1H), 6.70 (s, 1H), 6.39 (s, 1H), 6.29 (d, J=8.4 Hz, 1H), 5.23 (dd, J=12.5, 5.4 Hz, 1H), 4.57 (s, 1H), 4.34-4.29 (m, 1H), 4.23-4.15 (m, 2H), 3.28 (s, 3H), 3.03-2.90 (m, 4H), 2.87-2.75 (m, 3H), 2.70-2.55 (m, 3H), 2.28-2.21 (m 1H), 1.92-1.85 (m, 2H), 1.82-1.67 (m, 6H), 1.42 (s, 9H), 1.25 (d, J=3.2 Hz, 4H), 1.07-0.92 (m, 2H); LC/MS (ESI, m/z): [(M+1)]+=724.4.

### (5S,8S,10aR)-5-[(tert-butoxycarbonyl)amino]-3-(2-methylpropanoyl)-6-oxo-octahydropyrrolo[1,2-a][1,5]diazocine-8-carboxylic acid (Intermediate DK)

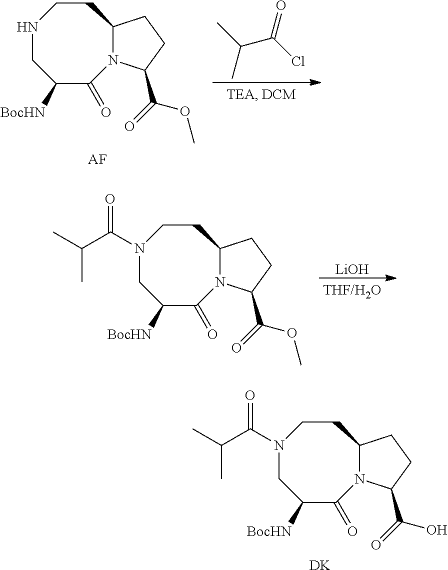

Step 1—Methyl (5S,8S,10aR)-5-[(tert-butoxycarbonyl)amino]-3-(2-methylpropanoyl)-6-oxo-octahydropyrrolo[1,2-a][1,5]diazocine-8-carboxylate. To a stirred solution of methyl (5S,8S,10aR)-5-[(tert-butoxycarbonyl)amino]-6-oxo-octahydro-1H-pyrrolo[1,2-a][1,5]diazocine-8-carboxylate (250.00 mg, 0.732 mmol, Intermediate AF) in DCM (2.00 mL) were added isobutyryl chloride (117.03 mg, 1.098 mmol) and TEA (0.3 mL, 2.2 mmol) in turns at rt under air atmosphere. The resulting mixture was stirred for 16 h at rt under air atmosphere. On completion, the reaction mixture was concentrated under reduced pressure. The residue was purified by reverse phase flash chromatography (Column: WelFlash™ C18-I, 20-40 um, 120 g; Eluent A: Water (10 mmol/L FA); Eluent B: ACN; Gradient: 30%-50% B in 25 min; Flow rate: 60 mL/min; Detector: 220/254 nm; desired fractions were collected at 42% B) and concentrated under reduced pressure to afford the title compound (279.5 mg, 93% yield) as a white solid. ¹H NMR (300 MHz, Chloroform-d) δ 8.10 (s, 1H), 5.84 (d, J=6.7 Hz, 1H), 4.52-4.46 (m, 2H), 4.14-4.09 (m, 1H), 4.01-3.96 (m, 1H), 3.91-3.87 (m, 1H), 3.76 (s, 3H), 3.40-3.06 (m, 2H), 2.43-2.28 (m, 1H), 2.22-1.95 (m, 3H), 1.84-1.79 (m, 2H), 1.45 (s, 9H), 1.29-1.14 (m, 6H); LC/MS (ESI, m/z): [(M+H)]+=412.3.

Step 2—(5S,8S,10aR)-5-[(tert-butoxycarbonyl)amino]-3-(2-methylpropanoyl)-6-oxo-octahydropyrrolo[1,2-a][1,5]diazocine-8-carboxylic acid. To a stirred solution of methyl (5S,8S,10aR)-5-[(tert-butoxycarbonyl)amino]-3-(2-methylpropanoyl)-6-oxo-octahydropyrrolo[1,2-a][1,5]diazocine-8-carboxylate (270 mg) in THF (5.00 mL) were added LiOH (130.14 mg, 5.43 mmol) and H2O (5.00 mL) dropwise at rt. The resulting mixture was stirred for 16 h at rt. On completion, the reaction mixture was concentrated under reduced pressure. The residue was purified by reverse phase flash chromatography (Column: WelFlash™ C18-I, 20-40 um, 120 g; Eluent A: Water (10 mmol/L FA); Eluent B: ACN; Gradient: 20%-40% B in 25 min; Flow rate: 60 mL/min; Detector: 220/254 nm; desired fractions were collected at 38% B) and concentrated under reduced pressure to afford the title compound (218 mg, 84% yield) as a white solid. ¹H NMR (300 MHz, Chloroform-d) δ 8.05 (s, 1H), 5.85 (d, J=6.8 Hz, 1H), 4.58-4.54 (m, 2H), 4.17-4.13 (m, 1H), 4.02-3.91 (m, 1H), 3.85-3.81 (m, 1H), 3.30-3.26 (m, 2H), 3.16-3.06 (m, 1H), 2.37-2.33 (m, 1H), 2.18-2.13 (m, 3H), 1.86-1.82 (m, 2H), 1.47 (s, 9H), 1.28-1.13 (m, 6H); LC/MS (ESI, m/z): [(M+H)]+=398.3.

### (2S)-2-amino-N-[(2-chloro-4-methanesulfonylphenyl)methyl]pentanediamide (Intermediate DL)

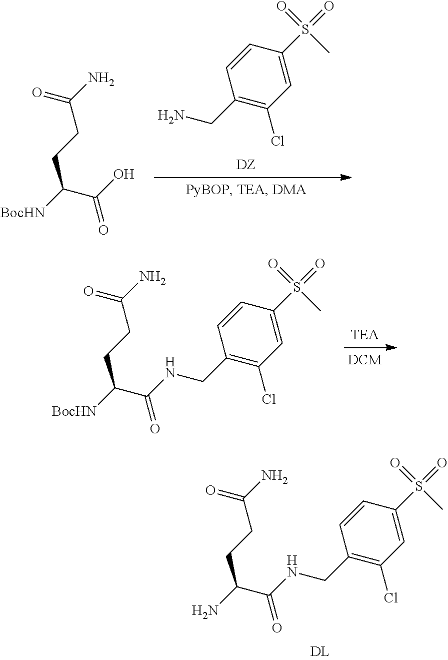 [SMILES: CC(C)(C)OC(=O)N[C@@H](CCC(N)=O)C(=O)O ; CS(=O)(=O)c1ccc(CN)c(Cl)c1 ; CC(C)(C)OC(=O)N[C@@H](CCC(N)=O)C(=O)NCc1ccc(S(C)(=O)=O)cc1Cl ; CS(=O)(=O)c1ccc(CNC(=O)[C@@H](N)CCC(N)=O)c(Cl)c1]

Step 1—Tert-butyl N-[(1S)-3-carbamoyl-1-[[(2-chloro-4-methanesulfonylphenyl)methyl]carbamoyl]propyl]carbamate. To a stirred mixture of (2S)-2-[(tert-butoxycarbonyl)amino]-4-carbamoylbutanoic acid (400.00 mg, 1.62 mmol) and 1-(2-chloro-4-methanesulfonylphenyl)methanamine (428.19 mg, 1.95 mmol, Intermediate DZ) in DMA (5.00 mL) were added PyBOP (1098.84 mg, 2.11 mmol) and TEA (493.08 mg, 4.87 mmol) in portions at rt under nitrogen atmosphere. The resulting mixture was stirred for 16 h at rt under nitrogen atmosphere. On completion, the residue was purified directly by reverse phase flash chromatography (Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 33% B—45% B gradient in 20 min; Detector: 220 nm; the fractions containing the desired product were collected at 39% B) and concentrated under reduced pressure to afford the title compound (700 mg, 96% yield) as a white solid. ¹H NMR (400 MHz, DMSO-d6) δ 8.57-8.51 (m, 1H), 7.99-7.95 (m, 1H), 7.85-7.78 (m, 1H), 7.63-7.58 (m, 1H), 7.35-7.28 (m, 1H), 7.15-7.08 (m, 1H), 6.81-6.75 (m, 1H), 4.46-4.31 (m, 2H), 3.95-3.91 (m, 1H), 3.28 (s, 3H), 2.20-2.09 (m, 2H), 1.97-1.69 (m, 2H), 1.41 (s, 9H); LC/MS (ESI, m/z): [(M+H)]+=448.1.

Step 2—(2S)-2-amino-N-[(2-chloro-4-methanesulfonylphenyl)methyl]pentanediamide; trifluoroacetic acid. To a stirred solution of tert-butyl N-[(1S)-3-carbamoyl-1-[[(2-chloro-4-methanesulfonylphenyl)methyl]carbamoyl]propyl]carbamate (700.00 mg, 1.56 mmol) in DCM (4.00 mL) was added TFA (20.00 mL) dropwise at 0° C. under nitrogen atmosphere. The resulting mixture was stirred for 2 h at rt under nitrogen atmosphere. On completion, the reaction mixture was concentrated under reduced pressure to afford the title compound (500 mg, 92% yield) as a white solid. ¹H NMR (300 MHz, DMSO-d6) δ 9.41-9.32 (m, 1H), 8.48-8.40 (m, 3H), 8.05-7.98 (m, 1H), 7.91-7.85 (m, 1H), 7.78-7.71 (m, 1H), 7.55-7.48 (m, 1H), 4.64-4.30 (m, 2H), 4.07-3.86 (m, 1H), 3.63 (s, 3H), 2.28-2.21 (m, 2H), 2.08-2.00 (m, 2H); LC/MS (ESI, m/z): [(M+H)]+=348.0.

### (2S)-2-amino-N-[(1S)-1-(4-methanesulfonylphenyl)ethyl]pentanediamide (Intermediate DM)

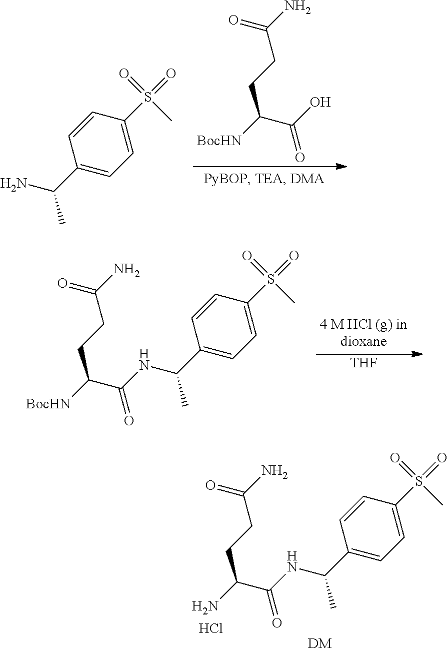

Step 1—Tert-butyl N-[(1S)-3-carbamoyl-1-[[(1S)-1-(4-methanesulfonylphenyl)ethyl]carbamoyl]propyl]carbamate. To a stirred mixture of (2S)-2-[(tert-butoxycarbonyl) amino]-4-carbamoylbutanoic acid (250.00 mg, 1.02 mmol, CAS #: 13726-85-7) and in DMA (4.00 mL) were added TEA (0.42 mL, 3.05 mmol) dropwise and (1S)-1-(4-methanesulfonylphenyl)ethanamine (242.75 mg, 1.22 mmol, CAS #: 1212202-62-4) in turns at rt under nitrogen atmosphere. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 m, 120 g; EluentA: Water (plus 10 mmol/L FA); Eluent B: ACN; Gradient: 25%-55% B in 25 min; Flow rate: 60 mL/min; Detector: 220/254 nm; desired fractions were collected at 42% B) and concentrated under reduced pressure to afford the title compound (290 mg, 67% yield) as a white solid. ¹H NMR (300 MHz, Chloroform-d) δ 7.91-7.86 (m, 2H), 7.55-7.50 (m, 2H), 7.47-7.42 (m, 1H), 6.13 (s, 1H), 5.74 (s, 1H), 5.17-5.06 (m, 2H), 4.19 (s, 1H), 3.04 (s, 3H), 2.50-2.38 (m, 1H), 2.34-2.20 (m, 1H), 2.11-2.01 (m, 1H), 1.95-1.85 (m, 1H), 1.49 (d, J=7.0 Hz, 3H), 1.44 (s, 9H); LC/MS (ESI, m/z): [(M+H)]+=428.2.

Step 2—(2S)-2-amino-N-[(1S)-1-(4-methanesulfonylphenyl)ethyl]pentanediamide hydrochloride. To a stirred solution of tert-butyl N-[(1S)-3-carbamoyl-1-[[(1S)-1-(4-methanesulfonylphenyl)ethyl]carbamoyl]propyl]carbamate (290.00 mg, 0.68 mmol) in THF (8.00 mL) was added 4 M HCl (gas) in 1,4-dioxane (8.00 mL) dropwise at rt. The resulting mixture was stirred for 2 h at rt under nitrogen atmosphere. On completion, the reaction mixture was concentrated under reduced pressure. The residue was triturated with Et₂O (30.00 mL) to afford the title compound (200 mg, 81% yield) as a white solid. ¹H NMR (300 MHz, DMSO-d6) δ 9.33-9.28 (m, 1H), 8.37-8.33 (m, 2H), 7.92-7.87 (m, 2H), 7.64-7.58 (m, 2H), 7.48 (s, 1H), 6.94 (s, 1H), 5.08-4.97 (m, 1H), 3.20 (s, 3H), 3.94-3.84 (m, 1H), 2.17-2.11 (m, 2H), 1.99-1.90 (m, 2H), 1.42 (d, J=7.0 Hz, 3H); LC/MS (ESI, m/z): [(M+H)]+=328.1.

### 3-[5-(3-bromopropyl)-3-methyl-2-oxo-1,3-benzodiazol-1-yl]piperidine-2,6-dione (Intermediate DN)

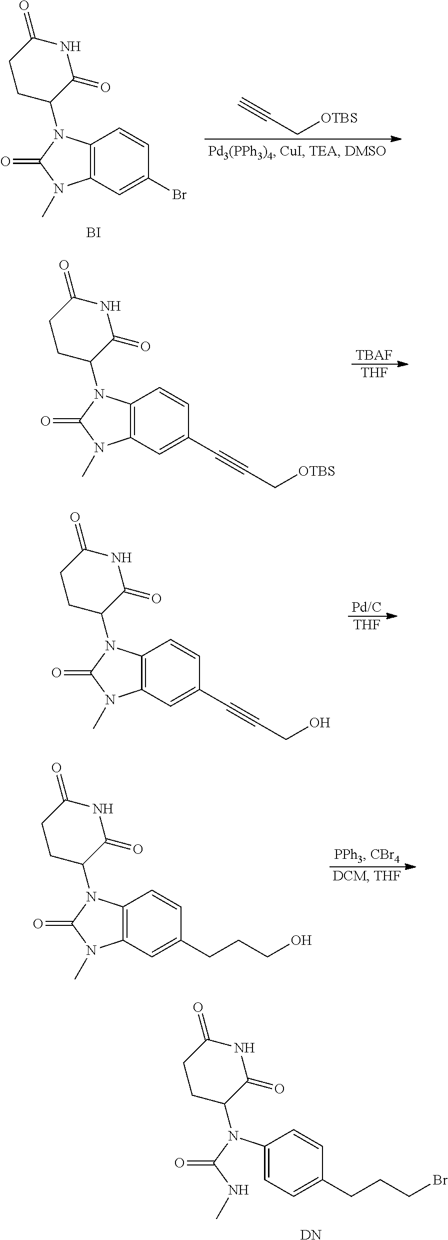

Step 1—3-(5-[3-[(tert-butyldimethylsilyl)oxy]prop-1-yn-1-yl]-3-methyl-2-oxo-2,3-dihydro-1H-1,3-benzodiazol-1-yl)piperidine-2,6-dione. To a stirred solution of 3-(5-bromo-3-methyl-2-oxo-2,3-dihydro-1H-1,3-benzodiazol-1-yl)piperidine-2,6-dione (8.38 g, 24.78 mmol, Intermediate BI) and tert-butyldimethyl(prop-2-yn-1-yloxy)silane (12.67 g, 74.39 mmol) in DMSO (36.00 mL) and TEA (18.00 mL, 129.50 mmol,) were added CuI (471.96 mg, 2.48 mmol) and Pd(PPh₃)₄ (2.87 g, 2.48 mmol) at rt. The resulting mixture was purged with nitrogen three times and stirred for 3 h at 85° C. under nitrogen atmosphere. On completion, the reaction was cooled to rt, then concentrated under reduced pressure. The mixture was then purified by reverse phase flash chromatography (Column: WelFlash™ C18-I, 20-40 m, 330 g; Eluent A: Water (plus 10 mmol/L FA); Eluent B: ACN; Gradient: 35%-70% B in 35 min; Flow rate: 80 mL/min; Detector: 220/254 nm; desired fractions were collected at 67% B) and concentrated under reduced pressure to afford the title compound (4.6 g, 43% yield) as a brown yellow solid. ¹H NMR (300 MHz, DMSO-d6) δ 11.12 (s, 1H), 7.30-7.27 (m, 1H), 7.14-7.12 (m, 2H), 5.42-5.35 (m, 1H), 4.55 (s, 2H), 3.34 (s, 3H), 2.94-2.59 (m, 4H), 0.90 (s, 9H), 0.14 (s, 6H); LC/MS (ESI, m/z): [(M+H)]+=428.2.

Step 2—3-[5-(3-hydroxyprop-1-yn-1-yl)-3-methyl-2-oxo-1,3-benzodiazol-1-yl]piperidine-2,6-dione. To a stirred mixture of 3-(5-[3-[(tert-butyldimethylsilyl)oxy]prop-1-yn-1-yl]-3-methyl-2-oxo-1,3-benzodiazol-1-yl)piperidine-2,6-dione (2.30 g, 5.38 mmol) and TBAF (2.81 g, 10.75 mmol) in THF (15.00 mL) at rt for 1 h under N₂ atmosphere. On completion, the reaction mixture was concentrated under reduced pressure. The residue was purified by silica gel column chromatography, eluted with EA:DCM (1:1), to afford the title compound (1.5 g, 89% yield) as a yellow solid. ¹H NMR (300 MHz, DMSO-d6) δ 11.03 (s, 1H), 7.08-6.92 (m, 2H), 6.86-6.79 (m, 1H), 5.35-5.4 (m, 1H), 4.46-4.37 (m, 1H), 3.60-3.52 (m, 2H), 3.27 (s, 3H), 2.90-2.54 (m, 4H); LC/MS (ESI, m/z): [(M+H)]+=314.1.

Step 3—3-[5-(3-hydroxypropyl)-3-methyl-2-oxo-1,3-benzodiazol-1-yl]piperidine-2,6-dione. To a stirred solution of 3-[5-(3-hydroxyprop-1-yn-1-yl)-3-methyl-2-oxo-1,3-benzodiazol-1-yl]piperidine-2,6-dione (1.00 g, 3.19 mmol) in THF (30.00 mL) was added Pd/C (10 wt %, 1.50 g) at rt under nitrogen atmosphere. The reaction system was degassed under vacuum and purged with H2 several times, then it was hydrogenated under H₂ balloon (˜1 atm) at 25° C. for 2 h. After completion of the reaction, Pd/C was filtered off through celite and the filter cake was washed with THF (3×10 mL). The filtrate was concentrated under reduced pressure to provide crude product. The residue was purified by reverse phase flash chromatography (Column: WelFlash™ C18-I, 20-40 m, 330 g; Eluent A: Water (plus 10 mmol/L FA); Eluent B: ACN; Gradient: 15%-35% B in 30 min; Flow rate: 80 mL/min; Detector: 220/254 nm; desired fractions were collected at 21% B) and concentrated under reduced pressure to afford the title compound (1.1 g, quant yield) as a yellow solid. ¹H NMR (300 MHz, DMSO-d6) δ11.07 (s, 1H), 7.06-6.96 (m, 2H), 6.90-6.82 (m, 1H), 5.45-5.17 (m, 2H), 3.57 (s, 3H), 3.45-3.40 (m, 2H), 3.33-3.31 (m, 2H), 2.95-2.58 (m, 4H), 1.83-1.63 (m, 2H); LC/MS (ESI, m/z): [(M+H)]+=318.2.

Step 4—3-[5-(3-bromopropyl)-3-methyl-2-oxo-1,3-benzodiazol-1-yl]piperidine-2,6-dione. To a stirred solution of 3-[5-(3-hydroxypropyl)-3-methyl-2-oxo-1,3-benzodiazol-1-yl]piperidine-2,6-dione (1.10 g, 3.47 mmol) and CBr₄ (3.45 g, 0.01 mmol) in DCM (15.00 mL) and THF (15.00 mL) was added PPh₃ (2.27 g, 0.009 mmol) at 25° C. under nitrogen atmosphere. The resulting mixture was stirred for 1 h at rt under nitrogen atmosphere. On completion, the reaction mixture was concentrated under reduced pressure. The residue was purified by silica gel column chromatography, eluted with EA:DCM:PE (2:1:1), to afford the title compound (1 g, 76% yield) as a white solid. ¹H NMR (400 MHz, DMSO-d6) δ 11.08 (s, 1H), 7.08-7.00 (m, 3H), 6.95-6.86 (m, 2H), 5.41-5.29 (m, 1H), 3.54-3.51 (m, 2H), 3.32 (s, 3H), 2.97-2.83 (m, 2H), 2.76-2.73 (m, 2H), 2.77-2.73 (m, 2H), 2.15-2.10 (m, 2H); LC/MS (ESI, m/z): [(M+H)]+=382.1.

### (4S)-4-amino-5-(2-chloro-3-[3-[1-(2,6-dioxopiperidin-3-yl)-3-methyl-2-oxo-1,3-benzodiazol-5-yl]propyl]phenoxy)pentanamide (Intermediate DO)

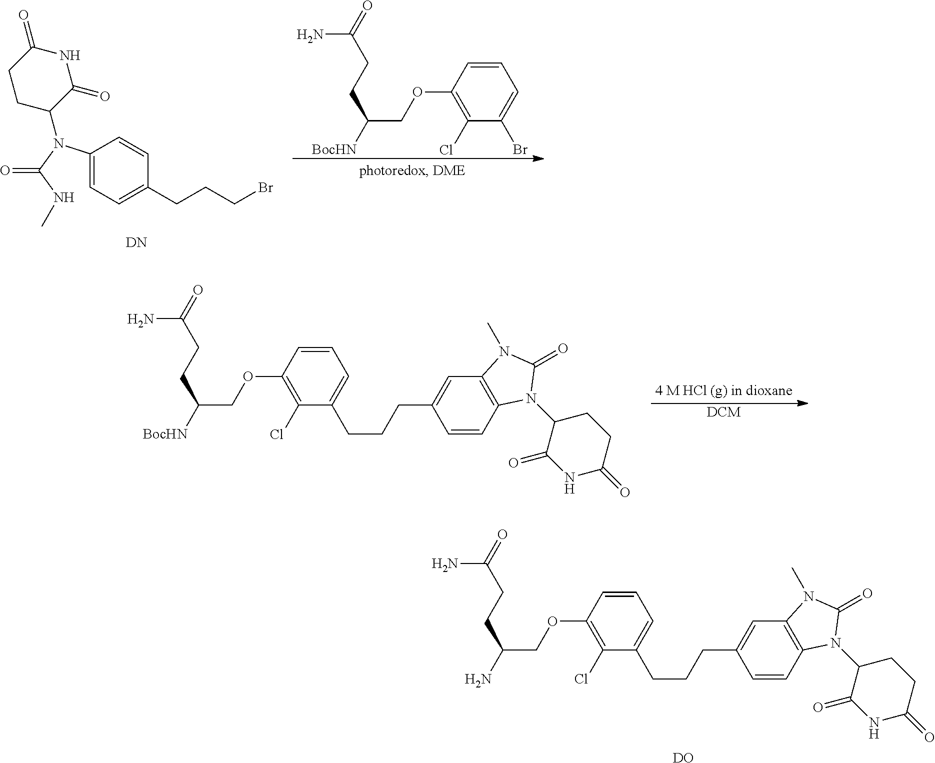

Step 1—Tert-butyl N-[(2S)-4-carbamoyl-1-(2-chloro-3-[3-[1-(2,6-dioxopiperidin-3-yl)-3-methyl-2-oxo-1,3-benzodiazol-5-yl]propyl]phenoxy)butan-2-yl]carbamate. To an 8 mL vial equipped with a stir bar was added photocatalyst Ir[dF(CF₃)ppy]₂(dtbbpy)PF₆ (172.61 mg, 0.154 mmol), 3-[5-(3-bromopropyl)-3-methyl-2-oxo-1,3-benzodiazol-1-yl]piperidine-2,6-dione (1.17 g, 3.077 mmol, Intermediate DN), tert-butyl N-[(2S)-1-(3-bromo-2-chlorophenoxy)-4-carbamoylbutan-2-yl]carbamate (1297.63 mg, 3.08 mmol, synthesized via Steps 1-3 of Intermediate C), tris(trimethylsilyl)silane (765.13 mg, 3.08 mmol), and anhydrous sodium carbonate (978.38 mg, 9.23 mmol). The vial was sealed and placed under nitrogen before 4 mL of DME was added. To a separate vial was added NiCl₂•glyme (67.61 mg, 0.308 mmol) and 4,4′-di-tert-butyl-2,2′-bipyridine (82.59 mg, 0.308 mmol). The catalyst vial was sealed, purged with nitrogen then to it was added 10 mL of DME. The precatalyst solution was sonicated or stirred for 5 min, after which, 10 mL (0.5 mol % catalyst, 2.5 mol, 0.005 equv.) was syringed into the reaction vessel. The solution was degassed by sparging with nitrogen while stirring for 10 minutes before sealing with Parafilm. The reaction was stirred and irradiated with a 34 W blue LED lamp (with cooling fan to keep the reaction temperature at 25° C.) for 16 hours. The reaction was quenched by exposure to air and concentrated in vacuo. The residue was purified by reverse phase flash chromatography (Column: WelFlash™ C18-I, 20-40 μm, 330 g; Eluent A: Water (plus 10 mmol/L FA); Eluent B: ACN; Gradient: 25%-50% B in 25 min; Flow rate: 80 mL/min; Detector: 220/254 nm; desired fractions were collected at 41% B) and concentrated under reduced pressure to afford the title compound (340 mg, 17% yield) as a yellow solid. ¹H NMR (300 MHz, DMSO-d6) δ 11.07 (s, 1H), 7.26-7.16 (m, 2H), 7.06-6.98 (m, 3H), 6.97-6.87 (m, 2H), 6.85-6.67 (m, 2H), 5.41-5.27 (m, 1H), 4.01-3.89 (m, 2H), 3.84-3.70 (m, 1H), 3.32 (s, 3H), 2.75-2.72 (m, 2H), 2.71-2.58 (m, 4H), 2.16-2.11 (m, 2H), 2.05-1.85 (m, 4H), 1.67-1.52 (m, 2H), 1.38 (s, 9H); LC/MS (ESI, m/z): [(M+H)]+=642.3.

Step 2—(4S)-4-amino-5-(2-chloro-3-[3-[1-(2,6-dioxopiperidin-3-yl)-3-methyl-2-oxo-1,3-benzodiazol-5-yl]propyl]phenoxy)pentanamide hydrochloride. To a stirred solution of tert-butyl N-[(2S)-4-carbamoyl-1-(2-chloro-3-{3-[1-(2,6-dioxopiperidin-3-yl)-3-methyl-2-oxo-1,3-benzodiazol-5-yl]propyl}phenoxy)butan-2-yl]carbamate (270 mg, 0.42 mmol) in DCM (15.00 mL) was added a solution of 4 M HCl (gas) in 1,4-dioxane (5 mL) at 0° C. under nitrogen atmosphere. The resulting mixture was stirred for 2 h at 25° C. under nitrogen atmosphere. On completion, the reaction mixture was concentrated under reduced pressure. The residue was purified by trituration with Et₂O (20.00 mL) to afford the title compound (290 mg, 95% yield) as a white solid. ¹H NMR (400 MHz, DMSO-d6) δ 11.08 (s, 1H), 8.37-8.32 (m, 2H), 7.49-7.42 (m, 1H), 7.29-7.21 (m, 1H), 7.06-7.03 (m, 2H), 7.02-7.00 (m, 1H), 6.91-6.84 (m, 1H), 5.39-5.31 (m, 1H), 4.28-4.12 (m, 2H), 3.57 (s, 3H), 3.33-3.32 (m, 2H), 2.96-2.85 (m, 1H), 2.78-2.59 (m, 6H), 2.38-2.23 (m, 2H), 2.04-1.84 (m, 6H); LC/MS (ESI, m/z): [(M+H)]+=542.3.

### 3-[3-methyl-2-oxo-5-(pent-4-en-1-yl)-1,3-benzodiazol-1-yl]piperidine-2,6-dione (Intermediate DP)

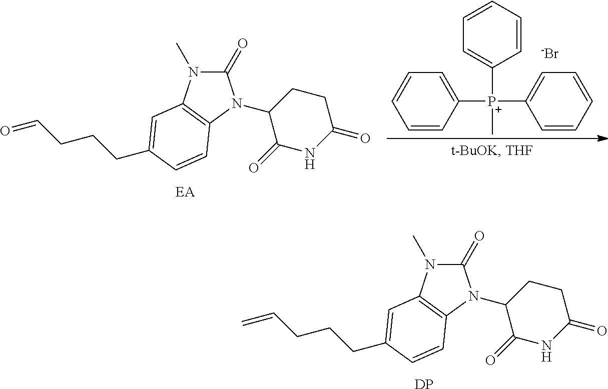

To a stirred solution of methyltriphenylphosphanium bromide (4.88 g, 13.66 mmol) in THF (30 mL) was added t-BuOK (10.02 mL, 10.02 mmol) dropwise at 0° C. under nitrogen atmosphere. The resulting mixture was stirred for 30 min at 0° C. under nitrogen atmosphere. To the above mixture was added 4-[1-(2,6-dioxopiperidin-3-yl)-3-methyl-2-oxo-1,3-benzodiazol-5-yl]butanal (3 g, 9.11 mmol, Intermediate EA) in portions at 0° C. The resulting mixture was stirred for an additional 2 h at rt. On completion, the reaction was quenched with water (80 mL) at rt. The resulting mixture was extracted with EA (3×200 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EA (10:1 to 1:1), to afford the title compound (2.2 g, 74% yield) as a light brown solid. ¹H NMR (400 MHz, Chloroform-d) δ 8.63 (s, 1H), 6.92-6.83 (m, 2H), 6.75-6.71 (m, 1H), 5.87-5.83 (m, 1H), 5.25-5.21 (m, 1H), 5.08-4.98 (m, 2H), 3.43 (s, 3H), 2.97-2.88 (m, 1H), 2.86-2.82 (m, 1H), 2.80-2.72 (m, 1H), 2.70-2.65 (m, 2H), 2.24-2.20 (m, 1H), 2.15-2.08 (m, 2H), 1.80-1.68 (m, 2H); LC/MS (ESI, m/z): [(M+H)]+=328.4.

### (4S)-4-amino-5-(2-chloro-3-{5-[1-(2,6-dioxopiperidin-3-yl)-3-methyl-2-oxo-1,3-benzodiazol-5-yl]pentyl}phenoxy)pentanamide (Intermediate DQ)

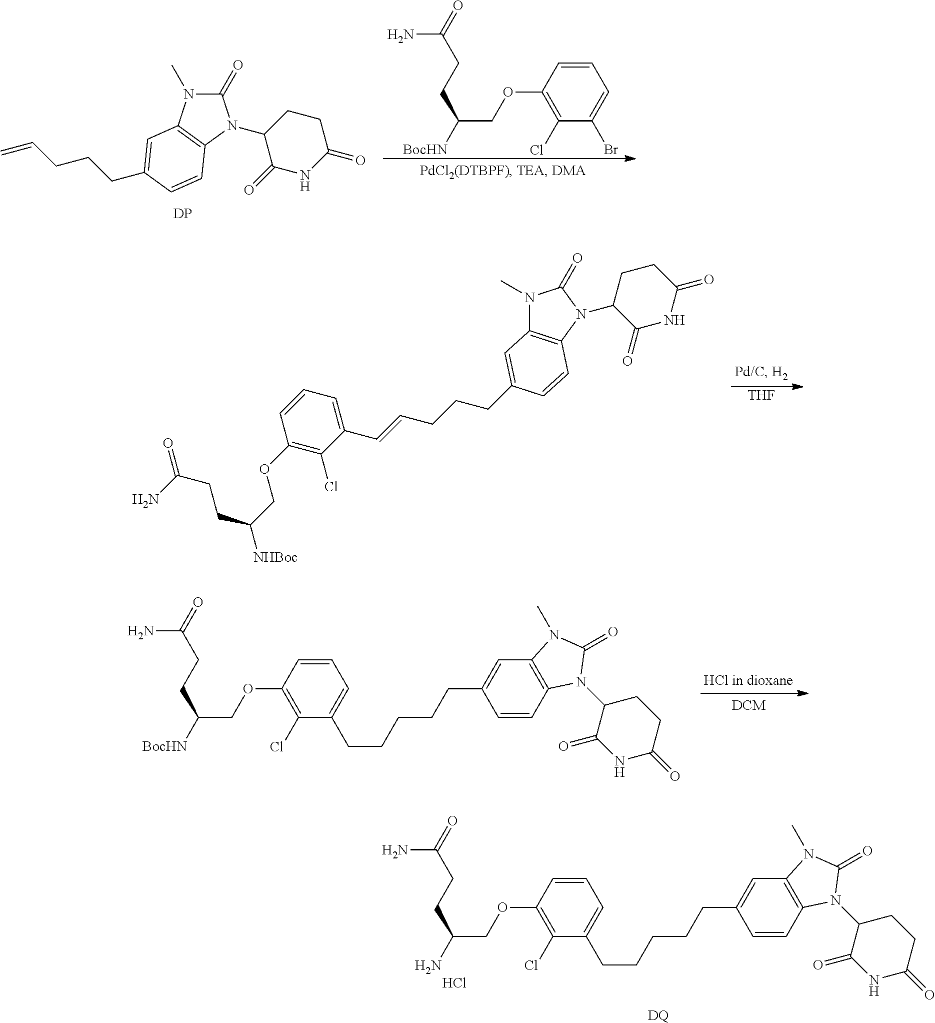

Step 1—Tert-butyl N-[(2S)-4-carbamoyl-1-{2-chloro-3-[(1E)-5-[1-(2,6-dioxopiperidin-3-yl)-3-methyl-2-oxo-1,3-benzodiazol-5-yl]pent-1-en-1-yl]phenoxy}butan-2-yl]carbamate. To a stirred solution of 3-[3-methyl-2-oxo-5-(pent-4-en-1-yl)-1,3-benzodiazol-1-yl]piperidine-2,6-dione (1.22 g, 3.73 mmol, Intermediate DP), Pd(DtBPF)Cl₂ (0.24 g, 0.37 mmol) and tert-butyl N-[(2S)-1-(3-bromo-2-chlorophenoxy)-4-carbamoylbutan-2-yl]carbamate (1.57 g, 3.73 mmol, synthesized via Steps 1-3 of Intermediate C) in DMA (15 mL) was added TEA (1.55 mL, 11.18 mmol) at rt under air atmosphere. The resulting mixture was stirred for 3 h at 100° C. under N2 atmosphere. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 50% B) and concentrated under reduced pressure to afford the title compound (780 mg, 31% yield) as a light brown solid. ¹H NMR (400 MHz, Chloroform-d) δ 8.59-8.53 (m, 1H), 7.18-7.14 (m, 1H), 6.96-6.69 (m, 4H), 6.45-6.41 (m, 1H), 5.74-5.70 (m, 1H), 5.28-5.19 (m, 1H), 4.08-4.04 (m, 3H), 3.47-3.37 (m, 3H), 2.96-2.92 (m, 1H), 2.89-2.80 (m, 1H), 2.79-2.67 (m, 2H), 2.43-2.29 (m, 3H), 2.26-2.22 (m, 2H), 2.09-2.03 (m, 4H), 1.90-1.82 (m, 1H), 1.48-1.43 (m, 13H); LC/MS (ESI, m/z): [(M+H)]+=668.3.

Step 2—Tert-butyl N-[(2S)-4-carbamoyl-1-(2-chloro-3-{5-[1-(2,6-dioxopiperidin-3-yl)-3-methyl-2-oxo-1,3-benzodiazol-5-yl]pentyl}phenoxy)butan-2-yl]carbamate. To a solution of tert-butyl N-[(2S)-4-carbamoyl-1-{2-chloro-3-[(1E)-5-[1-(2,6-dioxopiperidin-3-yl)-3-methyl-2-oxo-1,3-benzodiazol-5-yl]pent-1-en-1-yl]phenoxy}butan-2-yl]carbamate (780 mg, 1.17 mmol) in THF (10 mL) was added Pd/C (124.23 mg, 0.117 mmol) under nitrogen atmosphere. The reaction system was degassed under vacuum and purged with H₂ several times, then the reaction was hydrogenated under H₂ balloon (A1 atm) at 25° C. for 16 h. After completion of the reaction, Pd/C was filtered off through celite. The filter cake was washed with THF (3×50 mL). The filtrate was concentrated under reduced pressure to afford the title compound (660 mg, 84% yield) as a light brown solid. ¹H NMR (400 MHz, DMSO-d6) δ 11.07 (s, 1H), 7.46 (s, 1H), 7.25-7.21 (m, 1H), 7.05-6.95 (m, 3H), 6.94-6.90 (m, 1H), 6.89-6.78 (m, 1H), 5.37-5.33 (m, 1H), 4.25-4.22 (m, 1H), 4.17-4.13 (m, 1H), 3.87-3.81 (m, 3H), 3.32 (s, 3H), 2.93-2.89 (m, 1H), 2.73-2.69 (m, 2H), 2.66-2.59 (m, 4H), 2.34-2.30 (m, 2H), 2.00-1.96 (m, 4H), 1.65-1.56 (m, 3H), 1.49-1.40 (m, 11H); LC/MS (ESI, m/z): [(M+H)]+=670.2.

Step 3—(4S)-4-amino-5-(2-chloro-3-{5-[1-(2,6-dioxopiperidin-3-yl)-3-methyl-2-oxo-1,3-benzodiazol-5-yl]pentyl}phenoxy)pentanamide hydrochloride. To a stirred solution of tert-butyl N-[(2S)-4-carbamoyl-1-(2-chloro-3-{5-[1-(2,6-dioxopiperidin-3-yl)-3-methyl-2-oxo-1,3-benzodiazol-5-yl]pentyl}phenoxy)butan-2-yl]carbamate (660 mg, 0.99 mmol) in DCM (6.00 mL) was added HCl (gas) in 1,4-dioxane (1.5 mL) dropwise at rt under air atmosphere. The resulting mixture was stirred for 1 h at rt. On completion, the reaction mixture was concentrated under reduced pressure. The residue was purified by trituration with Et₂O (2×10 mL) to afford the title compound (700 mg, 94% yield) as a light brown solid. ¹H NMR (400 MHz, DMSO-d6) δ 11.07 (s, 1H), 7.46 (s, 1H), 7.25-7.21 (m, 1H), 7.05-6.95 (m, 3H), 6.94-6.90 (m, 1H), 6.89-6.78 (m, 1H), 5.37-5.33 (m, 1H), 4.25-4.22 (m, 1H), 4.17-4.13 (m, 1H), 3.87-3.81 (m, 3H), 3.32 (s, 3H), 2.93-2.89 (m, 1H), 2.73-2.69 (m, 2H), 2.66-2.59 (m, 4H), 2.34-2.30 (m, 2H), 2.00-1.96 (m, 4H), 1.65-1.56 (m, 4H), 1.40-1.36 (m, 2H); LC/MS (ESI, m/z): [(M+H)]+=570.2.

### (2S,4R)-4-hydroxy-N-[[4-(4-methyl-1,3-thiazol-5-yl)phenyl]methyl]pyrrolidine-2-carboxamide hydrochloride (Intermediate DR)

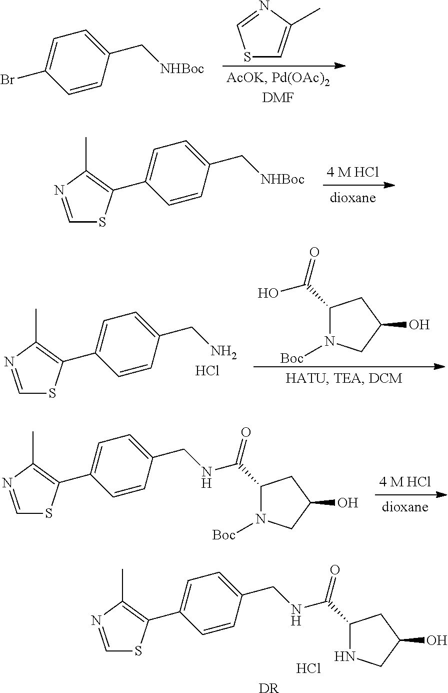

Step 1—Tert-butyl N-[[4-(4-methyl-1,3-thiazol-5-yl)phenyl]methyl]carbamate. To a solution of tert-butyl N-[(4-bromophenyl)methyl]carbamate (136 g, 475 mmol) and 4-methylthiazole (56.54 g, 570 mmol) in DMF (1200 mL) were added AcOK (93.28 g, 950 mmol) and Pd(OAc)₂ (10.67 g, 47.52 mmol) under nitrogen atmosphere at 25° C. The mixture was stirred at 90° C. for 16 h. On completion, the resulting mixture was filtered and the filter cake was washed with EtOAc (3×300 mL). The resulting mixture was diluted with H₂O (500 mL), then extracted with EtOAc (3×500 mL). The combined organic layers were washed with brine (3×300 mL), and dried over anhydrous Na2SO4. After filtration, the filtrate was concentrated under reduced pressure. The residue was purified by silica gel column chromatography, eluted with CH₂C1₂/MeOH (20:1 to 10:1), to afford the title compound (96 g, 66% yield) as a yellow solid. 1H NMR (400 MHz, Chloroform-d) δ 8.68 (s, 1H), 7.50-7.34 (m, 5H), 4.41-4.33 (m, 2H), 2.54 (s, 3H), 1.49 (s, 9H). LC/MS (ESI, m/z): [(M+H)]+=305.5.

Step 2—1-[4-(4-methyl-1,3-thiazol-5-yl)phenyl]methanamine hydrochloride. To a solution of tert-butyl N-[[4-(4-methyl-1,3-thiazol-5-yl)phenyl]methyl]carbamate (96.00 g, 315.36 mmol) in 1,4-dioxane (500.00 mL) was added 4 M HCl (gas) in 1,4-dioxane (500.00 mL) dropwise at 0° C. The mixture was then stirred at 25° C. for 16 h. On completion, the reaction mixture was concentrated under reduced pressure to afford the title compound (80 g) as a yellow solid. ¹H NMR (400 MHz, Methanol-d4) δ 7.81-7.70 (m, 5H) 4.28-4.21 (m, 2H), 2.65 (s, 3H). LC/MS (ESI, m/z): [(M+H)]+=205.3.

Step 3—tert-butyl (2S,4R)-4-hydroxy-2-([[4-(4-methyl-1,3-thiazol-5-yl)phenyl]methyl]carbamoyl)pyrrolidine-1-carboxylate. To a solution of 1-[4-(4-methyl-1,3-thiazol-5-yl)phenyl]methanamine hydrochloride (95.00 g, 394.60 mmol) and (2S,4R)-1-(tert-butoxycarbonyl)-4-hydroxypyrrolidine-2-carboxylic acid (91.25 g, 394.6 mmol, CAS #13726-69-7) in DCM (1200 mL) were added HATU (180.05 g, 473.5 mmol) and TEA (164.5 mL, 1183 mmol) at 0° C. The mixture was then stirred at 25° C. for 2 h. The resulting mixture was quenched with H₂O (500 mL) and the mixture was extracted with CH₂C1₂ (3×500 mL). The combined organic layers were washed with brine (3×300 mL), and dried over anhydrous Na₂SO₄. After filtration, the filtrate was concentrated under reduced pressure. The residue was purified by silica gel column chromatography, eluted with CH₂C1₂/MeOH (20:1 to 10:1), to afford the title compound (160 g, 97% yield) as a yellow solid. ¹H NMR (400 MHz, DMSO-d6) δ 8.99 (s, 1H), 8.87-8.82 (m, 1H), 8.51-8.45 (m, 1H), 7.47-7.34 (m, 4H), 5.05-5.00 (m, 1H), 4.38-4.27 (m, 2H), 3.44-3.25 (m, 2H), 2.45 (s, 3H), 2.08-1.87 (m, 2H), 1.42-1.26 (m, 1H), 1.18 (s, 9H); LC/MS (ESI, m/z): [(M+H)]+=418.2.

Step 4—(2S,4R)-4-hydroxy-N-[[4-(4-methyl-1,3-thiazol-5-yl)phenyl]methyl]pyrrolidine-2-carboxamide hydrochloride. To a solution of tert-butyl (2S,4R)-4-hydroxy-2-([[4-(4-methyl-1,3-thiazol-5-yl)phenyl]methyl]carbamoyl)pyrrolidine-1-carboxylate (160.00 g, 383.2 mmol) in 1,4-dioxane (500.00 mL) was added 4 M HCl (gas) in 1,4-dioxane (500.00 mL) at 0° C. The mixture was then stirred at rt for 16 h. On completion, the mixture was concentrated under reduced pressure. The residue was triturated with Et₂O. The solid was collected by filtration and dried under vacuum to afford the title compound (120 g, 89% yield) as a yellow solid. ¹H NMR (400 MHz, DMSO-d6) δ 10.28 (s, 1H), 9.41-9.36 (m, 1H), 9.26-9.20 (m, 1H), 8.71-8.65 (m, 1H), 7.54-7.46 (m, 2H), 7.54-7.46 (m, 2H), 4.48-4.38 (m, 3H), 3.38-3.31 (m, 1H), 3.06-3.01 (m, 2H), 2.48 (s, 3H), 2.01-1.87 (m, 1H), 1.25-1.19 (m, 1H); LC/MS (ESI, m/z): [(M+H)]+=318.2.

### 3-[5-(5-hydroxypentyl)-3-methyl-2-oxo-1,3-benzodiazol-1-yl]piperidine-2,6-dione (Intermediate DS)

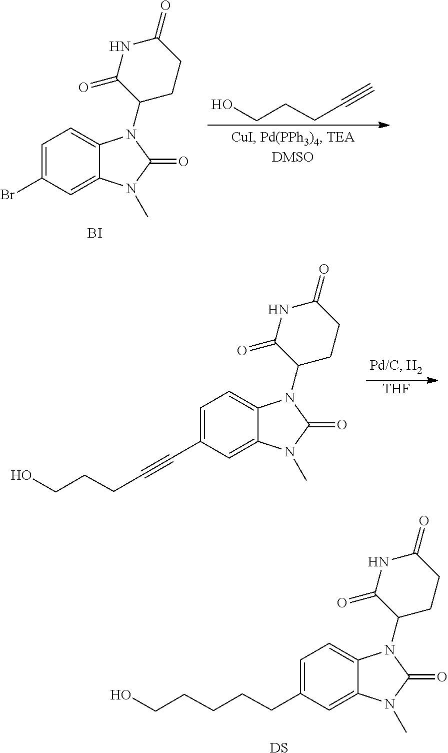

Step 1—3-[5-(5-hydroxypent-1-yn-1-yl)-3-methyl-2-oxo-1,3-benzodiazol-1-yl]piperidine-2,6-dione. To a stirred mixture of 3-(5-bromo-3-methyl-2-oxo-1,3-benzodiazol-1-yl)piperidine-2,6-dione (12.00 g, 35.49 mmol, Intermediate BI) and pent-4-yn-1-ol (8.96 g, 106 mmol)) in TEA (30.00 mL)/DMSO (60.00 mL) was added CuI (675.83 mg, 3.549 mmol) and Pd(PPh₃)₄ (4.10 g, 3.55 mmol) at rt under nitrogen atmosphere. The mixture was stirred for 3 h at 90° C. under nitrogen atmosphere. On completion, the reaction mixture was cooled to rt., filtered, and the filter cake was washed with CH₂C1₂ (2×20 mL). The filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 20%-45% B in 20 min; Flow rate: 80 mL/min; Detector: 220/254 nm; desired fractions were collected at 29% B) and concentrated under reduced pressure to afford the title compound (9.0 g, 74% yield) as a white solid. ¹H NMR (400 MHz, DMSO-d6) δ 11.10 (s, 1H), 7.24 (d, J=1.1 Hz, 1H), 7.11-7.07 (m, 2H), 5.37 (dd, J=12.8, 5.3 Hz, 1H), 4.52 (t, J=5.2 Hz, 1H), 3.55-3.51 (m, 2H), 3.35 (s, 3H), 2.96-2.80 (m, 1H), 2.77-2.57 (m, 2H), 2.46 (t, J=7.1 Hz, 2H), 2.09-1.94 (m, 1H), 1.77-1.66 (m, 2H); LC/MS (ESI, m/z): [(M+H)]+=342.2.

Step 2—3-[5-(5-hydroxypentyl)-3-methyl-2-oxo-1,3-benzodiazol-1-yl]piperidine-2,6-dione. To a solution of 3-[5-(5-hydroxypent-1-yn-1-yl)-3-methyl-2-oxo-1,3-benzodiazol-1-yl]piperidine-2,6-dione (9.00 g, 26.37 mmol) in THF (120.00 mL) was added Pd/C (2.81 g, 2.64 mmol, 10 wt %) under nitrogen atmosphere in a 250 mL round-bottom flask. The mixture was hydrogenated at rt for 4 h under hydrogen atmosphere using a hydrogen balloon. On completion, the reaction mixture was filtered through a celite pad and concentrated under reduced pressure to afford the title compound (8.5 g, 93% yield) as a yellow solid. ¹H NMR (400 MHz, DMSO-d6) δ 11.10 (s, 1H), 7.00-6.95 (m, 2H), 6.89-6.85 (m, 1H), 5.37 (dd, J=12.5, 5.4 Hz, 1H), 4.37 (t, J=5.1 Hz, 1H), 3.58 (s, 3H), 3.43-3.38 (m, 2H), 2.91-2.87 (m, 3H), 2.69-2.65 (m, 2H), 2.08-1.91 (m, 1H), 1.64-1.58 (m, 2H), 1.54-1.36 (m, 4H); LC/MS (ESI, m/z): [(M+H)]+=346.2.

### 5-[3-[(2R)-2-[(tert-butoxycarbonyl)amino]-4-carbamoylbutoxy]-2-chlorophenyl]pentanoic acid (Intermediate DT)

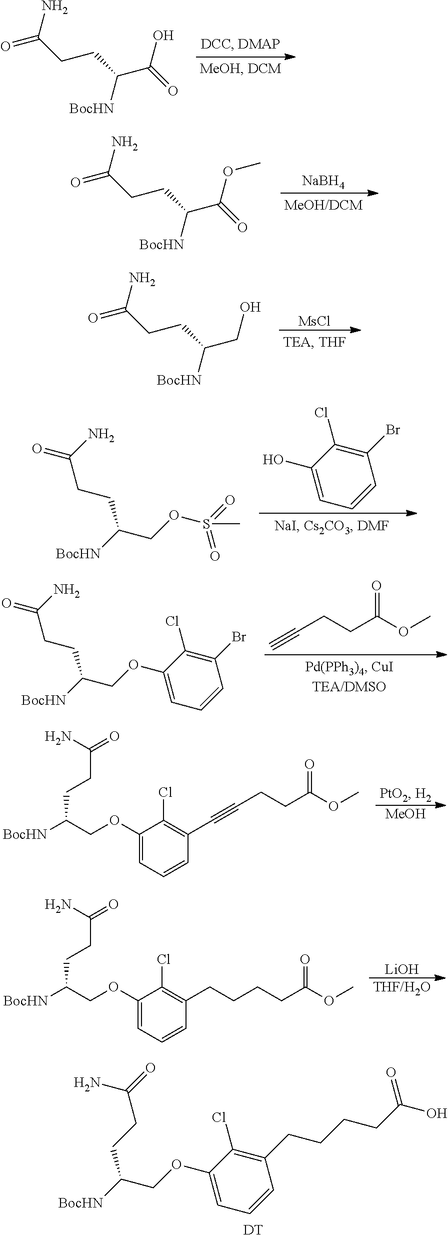

Step 1—Methyl (2R)-2-[(tert-butoxycarbonyl)amino]-4-carbamoylbutanoate. To a stirred solution of (2R)-2-[(tert-butoxycarbonyl)amino]-4-carbamoylbutanoic acid (30.00 g, 121.82 mmol, CAS #: 61348-28-5) and MeOH (24.7 mL, 609.202 mmol) in DCM (500.00 mL) were added DCC (30.16 g, 146.2 mmol) and DMAP (1.49 g, 12.2 mmol) at 25° C. under nitrogen atmosphere. The resulting mixture was stirred for 16 h at rt under nitrogen atmosphere. On completion, the reaction mixture was filtered, then the filter cake was washed with DCM (100 mL×3). The filtrate was concentrated under reduced pressure. The residue was purified by silica gel column chromatography, eluted with CH₂C1₂/MeOH (40:1), to afford the title compound (30 g, 76% yield) as a white solid. ¹H NMR (400 MHz, Methanol-d4) δ 4.18-4.14 (m, 1H), 3.74 (s, 3H), 2.16-2.04 (m, 1H), 1.97-1.83 (m, 2H), 1.78-1.65 (m, 1H), 1.46 (s, 9H); LC/MS (ESI, m/z): [(M+1)]+=261.1.

Step 2—tert-butyl N-[(2R)-4-carbamoyl-1-hydroxybutan-2-yl]carbamate. To a stirred mixture of methyl (2R)-2-[(tert-butoxycarbonyl)amino]-4-carbamoylbutanoate (30.00 g, 115.3 mmol) in MeOH (150.00 mL) and THF (150.00 mL) was added NaBH₄ (8.72 g, 231 mmol) at 0° C. under nitrogen atmosphere. The resulting mixture was stirred for 16 h at rt under nitrogen atmosphere. On completion, the reaction mixture was concentrated under reduced pressure. The residue was purified by silica gel column chromatography, eluted with CH2Cl2/MeOH (10:1), to afford the title compound (20 g, 75% yield) as a white solid. ¹H NMR (300 MHz, DMSO-d6) δ 7.23 (s, 1H), 6.69 (s, 1H), 6.52-6.43 (m, 1H), 4.59-4.52 (m, 1H), 3.31-3.17 (m, 2H), 2.12-1.96 (m, 2H), 1.81-1.66 (m, 1H), 1.49-1.42 (m, 1H), 1.39 (s, 9H); LC/MS (ESI, m/z): [(M+1)]+=261.1.

Step 3—tert-butyl N-[(2R)-4-carbamoyl-1-(methanesulfonyloxy)butan-2-yl]carbamate. To a stirred mixture of tert-butyl N-[(2R)-4-carbamoyl-1-hydroxybutan-2-yl]carbamate (10.00 g, 43.05 mmol) and TEA (11.96 g, 86.08 mmol) in THF (150.00 mL) was added a solution of Ms-C1 (5.92 g, 51.7 mmol) in THF (150.00 mL) dropwise at 0° C. under nitrogen atmosphere. The resulting mixture was stirred for 3 h at rt under nitrogen atmosphere. On completion, the reaction mixture was acidified to pH 6 with 1 M HCl (aq.). The resulting mixture was diluted with water (500 mL) and was then extracted with EtOAc (3×100 mL). The combined organic layers were washed with brine (2×100 mL), and dried over anhydrous Na2SO4. After filtration, the filtrate was concentrated under reduced pressure to afford the title compound (10 g, 75% yield) as a white solid. ¹H NMR (300 MHz, Methanol-d4) δ 4.24-4.13 (m, 2H), 3.89-3.78 (m, 1H), 3.09 (s, 3H), 2.38-2.23 (m, 2H), 1.97-1.83 (m, 1H), 1.82-1.69 (m, 1H), 1.46 (s, 9H); LC/MS (ESI, m/z): [(M+1)]+=311.0.

Step 4—tert-butyl N-[(2R)-1-(3-bromo-2-chlorophenoxy)-4-carbamoylbutan-2-yl]carbamate. To a stirred solution of tert-butyl N-[(2R)-4-carbamoyl-1-(methanesulfonyloxy)butan-2-yl]carbamate (5.00 g, 16.11 mmol) and 3-bromo-2-chlorophenol (3.34 g, 16.1 mmol) in DMF (40.00 mL) were added Cs₂CO₃ (10.50 g, 32.23 mmol) and NaI (1.21 g, 8.07 mmol) at rt under nitrogen atmosphere. The resulting mixture was stirred for 16 h at 70° C. under nitrogen atmosphere. On completion, the mixture was allowed to cool to rt. The resulting mixture was diluted with water (500 mL) and was extracted with EtOAc (3×100 mL). The combined organic layers were washed with brine (2×100 mL), and dried over anhydrous Na2SO4. After filtration, the filtrate was concentrated under reduced pressure. The residue was purified by silica gel column chromatography, eluted with CH₂C1₂/MeOH (10:1), to afford the title compound (4.6 g, 68% yield) as a light yellow oil. ¹H NMR (400 MHz, Chloroform-d) δ 7.23 (dd, J=8.2, 1.3 Hz, 1H), 7.06 (d, J=8.2 Hz, 1H), 6.86 (dd, J=8.3, 1.3 Hz, 1H), 6.48 (s, 1H), 5.65 (s, 1H), 5.23 (s, 1H), 4.07-3.95 (m, 3H), 2.42-2.31 (m, 2H), 2.07-1.96 (m, 2H), 1.43 (s, 9H); LC/MS (ESI, m/z): [(M+1)]+=421.1, 423.0.

Step 5—methyl 5-[3-[(2R)-2-[(tert-butoxycarbonyl)amino]-4-carbamoylbutoxy]-2-chlorophenyl]pent-4-ynoate. To a stirred solution of tert-butyl N-[(2R)-1-(3-bromo-2-chlorophenoxy)-4-carbamoylbutan-2-yl]carbamate (3.50 g, 8.30 mmol) and methyl pent-4-ynoate (2.79 g, 24.9 mmol) in DMSO (20.00 mL) and TEA (14.00 mL, 138.4 mmol) were added Pd(PPh₃)₄ (0.96 g, 0.83 mmol) and CuI (0.16 g, 0.83 mmol) at rt under nitrogen atmosphere. The resulting mixture was stirred for 3 h at 85° C. under nitrogen atmosphere. On completion, the reaction mixture was cooled to rt and the mixture was concentrated under reduced pressure. The residue was purified by reverse phase flash chromatography (Column: WelFlash™ C18-I, 20-40 m, 330 g; Eluent A: Water (plus 10 mmol/L NH4HCO3); Eluent B: ACN; Gradient: 35%-55% B in 20 min; Flow rate: 80 mL/min; Detector: 220/254 nm; desired fractions were collected at 45% B) and concentrated under reduced pressure to afford the title compound (1.97 g, 52% yield) as a white solid. ¹H NMR (300 MHz, Chloroform-d) δ 7.19-7.05 (m, 2H), 6.90-6.81 (m, 1H), 6.29 (s, 1H), 5.59 (s, 1H), 5.15 (s, 1H), 4.13-3.97 (m, 3H), 3.75 (s, 3H), 2.86-2.78 (m, 2H), 2.73-2.66 (m, 2H), 2.61-2.46 (m, 1H), 2.23-2.06 (m, 2H), 1.95-1.78 (m, 1H), 1.48 (s, 9H); LC/MS (ESI, m/z): [(M+1)]+=453.1.

Step 6—methyl 5-[3-[(2R)-2-[(tert-butoxycarbonyl)amino]-4-carbamoylbutoxy]-2-chlorophenyl]pentanoate. To a stirred solution of methyl 5-[3-[(2R)-2-[(tert-butoxycarbonyl)amino]-4-carbamoylbutoxy]-2-chlorophenyl]pent-4-ynoate (1.97 g, 4.35 mmol) in MeOH (30.00 mL) was added PtO₂ (148 mg, 0.652 mmol) at rt under nitrogen atmosphere. The resulting mixture was stirred for 3 h at 25° C. under hydrogen atmosphere. On completion, the reaction mixture was filtered, then the filter cake was washed with MeOH (3×5.00 mL). The filtrate was concentrated under reduced pressure to afford the title compound (1.84 g, 93% yield) as a yellow oil. ¹H NMR (300 MHz, Chloroform-d) δ 7.14 (d, J=7.9 Hz, 1H), 6.87 (d, J=7.6 Hz, 1H), 6.82-6.70 (m, 1H), 6.40 (s, 1H), 5.48 (s, 1H), 5.19 (s, 1H), 4.13-3.98 (m, 3H), 3.69 (s, 3H), 2.82-2.73 (m, 2H), 2.43-2.33 (m, 4H), 2.14-2.02 (m, 2H), 1.79-1.59 (m, 4H), 1.48 (s, 9H); LC/MS (ESI, m/z): [(M+1)]+=457.1.

Step 7—5-[3-[(2R)-2-[(tert-butoxycarbonyl)amino]-4-carbamoylbutoxy]-2-chlorophenyl]pentanoic acid. To a stirred solution of methyl 5-[3-[(2R)-2-[(tert-butoxycarbonyl)amino]-4-carbamoylbutoxy]-2-chlorophenyl]pentanoate (1.84 g, 4.03 mmol) in THF (20 mL) was added a solution of LiOH (0.96 g, 40.27 mmol) in H₂O (10.00 mL) at rt under nitrogen atmosphere. The resulting mixture was stirred for 4 h at 25° C. under nitrogen atmosphere. On completion, the reaction mixture was acidified to pH 5 with HCl (aq.), then the mixture was concentrated under reduced pressure. The solution was purified by reverse phase flash chromatography (Column: WelFlash™ C18-I, 20-40 μm, 330 g; Eluent A: Water (plus 10 mmol/L FA); Eluent B: ACN; Gradient: 30%-60% B in 30 min; Flow rate: 80 mL/min; Detector: 220/254 nm; desired fractions were collected at 54% B) and concentrated under reduced pressure to afford the title compound (1.23 g, 68% yield) as a white solid. ¹H NMR (400 MHz, DMSO-d6) δ 11.99 (s, 1H), 7.26 (s, 1H), 7.22-7.16 (m, 1H), 7.01-6.94 (m, 1H), 6.93-6.88 (m, 1H), 6.81 (d, J=8.4 Hz, 1H), 6.73 (s, 1H), 3.92 (d, J=6.1 Hz, 2H), 3.81-3.72 (m, 1H), 2.73-2.64 (m, 3H), 2.28-2.19 (m, 3H), 2.18-2.11 (m, 1H), 1.90-1.78 (m, 1H), 1.67-1.48 (m, 4H), 1.39 (s, 9H); LC/MS (ESI, m/z): [(M+1)]+=443.1.

### (4R)-4-aminopentanamide (Intermediate DU)

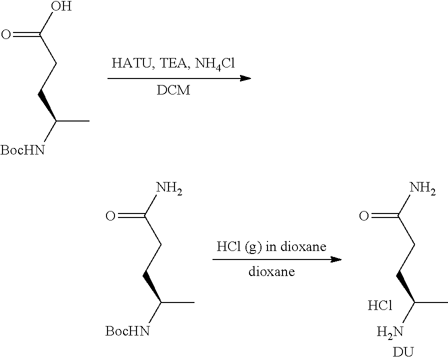

Step 1—Tert-butyl N-[(2R)-4-carbamoylbutan-2-yl]carbamate. To a stirred solution of (4R)-4-[(tert-butoxycarbonyl)amino]pentanoic acid (CAS #: 214402-34-3) (3.70 g, 17.0 mmol) in DCM (40.00 mL) were added TEA (9.46 mL, 68.1 mmol) and HATU (8.42 g, 22.1 mmol) and NH₄Cl (1.09 g, 20.4 mmol) in turns at rt under N₂ atmosphere. The resulting mixture was stirred for 2 h at rt under N2 atmosphere. On completion, the reaction mixture was concentrated under vacuum. The residue was purified by silica gel column chromatography, eluted with PE/EtOAc (30%˜70%), to afford the title compound (2.9 g, 79% yield) as a white solid. ¹H NMR (300 MHz, Chloroform-d) δ 6.54 (s, 1H), 5.56 (s, 1H), 4.42 (d, J=58.2 Hz, 1H), 3.73 (s, 1H), 2.36-2.22 (m, 2H), 1.88-1.62 (m, 2H), 1.46 (s, 9H), 1.18 (d, J=6.6 Hz, 3H); LC/MS (ESI, m/z): [(M+H)]+=217.2.

Step 2—(4R)-4-aminopentanamide hydrochloride. To a stirred solution of tert-butyl N-[(2R)-4-carbamoylbutan-2-yl]carbamate (2.90 g, 13.4 mmol) in dioxane (15.00 mL) was added 4 M HCl (gas) in 1,4-dioxane (15.00 mL) dropwise rt. The resulting mixture was stirred for 2 h at rt. On completion, the reaction mixture was concentrated under reduced pressure. The residue was triturated with Et2O to afford the title compound (2.2 g) as a yellow solid. 1H NMR (300 MHz, DMSO-d6) δ 8.24 (broad, 2H), 6.20 (s, 2H), 3.22-3.03 (m, 1H), 2.30-2.10 (m, 2H), 1.91-1.85 (m, 1H), 1.72-1.58 (m, 1H), 1.20 (d, J=6.5 Hz, 3H); LC/MS (ESI, m/z): [(M+H)]+=117.2.

### Tert-butyl N-[(2S,11S)-6-bromo-2-[[(2R)-4-carbamoylbutan-2-yl]carbamoyl]-12-oxo-1-azatricyclo[6.4.1.0\hat{ }[4,13]]trideca-4(13),5,7-trien-11-yl]carbamate (Intermediate DV)

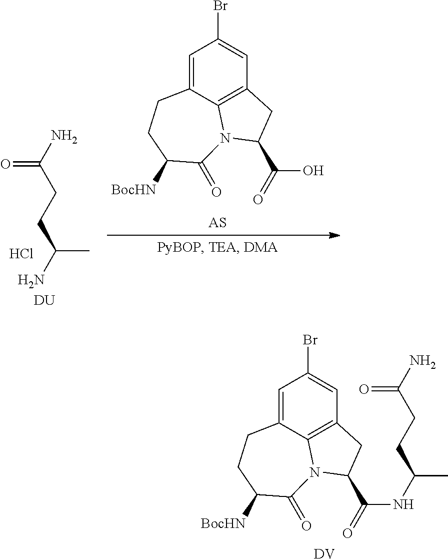

To a stirred solution of (4R)-4-aminopentanamide hydrochloride (1.00 g, 6.55 mmol, Intermediate DU) and (2S,11S)-6-bromo-11-[(tert-butoxycarbonyl)amino]-12-oxo-1-azatricyclo[6.4.1.0\hat{ }[4,13]]trideca-4(13),5,7-triene-2-carboxylic acid (3.34 g, 7.86 mmol, Intermediate AS) in DMA (10.00 mL) were added TEA (4.56 mL, 32.8 mmol) and PyBOP (5.11 g, 9.83 mmol) in turns at rt under air atmosphere. The resulting mixture was stirred for 16 h at rt. On completion, the reaction mixture was concentrated under reduced pressure. The crude product was purified by reverse phase flash (Column: Spherical C18 Column, 20-40 um, 330 g; Mobile Phase A: Water (0.1% NH₄HCO₃), Mobile Phase B: ACN; Flow rate: 80 mL/min; Gradient: 35% B to 55% B in 25 min, 254 nm; the fractions containing the desired product were collected at 47% B) to afford the title compound (1.46 g, 43% yield) as a white solid. ¹H NMR (300 MHz, Chloroform-d) δ 7.25-7.21 (m, 2H), 6.70 (d, J=8.5 Hz, 1H), 6.10 (s, 1H), 5.81 (s, 1H), 5.20-5.16 (m, 2H), 4.29-4.25 (m, 1H), 4.01-3.97 (m, 1H), 3.50-3.45 (m, 1H), 3.35-2.99 (m, 3H), 2.42-2.37 (m, 1H), 2.19-2.13 (m, 3H), 1.88-1.62 (m, 2H), 1.48 (s, 9H), 1.21 (d, J=6.6 Hz, 3H); LC/MS (ESI, m/z): [(M+H)]+=523.2.

### (4S)-4-amino-4-(1,3-benzothiazol-2-yl) butanamide (Intermediate DW)

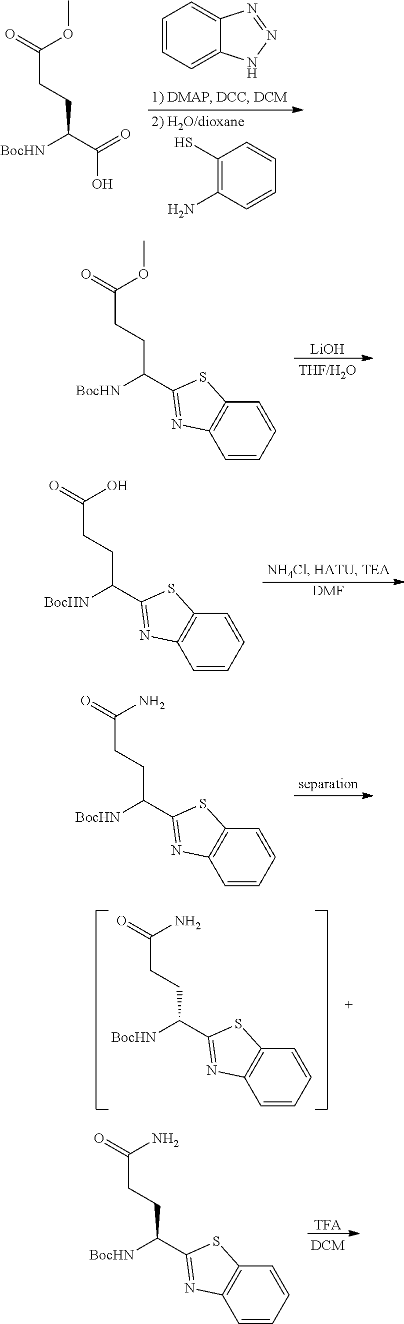

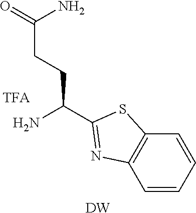 [SMILES: NC(=O)CC[C@H](N)c1nc2ccccc2s1 ; "DW"]

Step 1—Methyl 4-(benzo[d]thiazol-2-yl)-4-((tert-butoxycarbonyl)amino)butanoate. To a stirred solution of (2S)-2-[(tert-butoxycarbonyl)amino]-5-methoxy-5-oxopentanoic acid (10.00 g, 38.27 mmol) and benzotriazole (4.56 g, 38.3 mmol) in DCM (40.00 mL) were added DMAP (0.23 g, 1.88 mmol) and DCC (5.92 g, 28.7 mmol) in turns at 0° C. under nitrogen atmosphere. The resulting mixture was stirred for 2 h at 25° C. under nitrogen atmosphere. On completion, the reaction mixture was concentrated under reduced pressure. The residue was purified by reverse flash chromatography (Column, C18 silica gel; mobile phase, MeCN in water (10 mmol/L NH4HCO3), 20% to 42% gradient in 30 min; detector, UV 220 nm) to give the activated ester. Then the activated ester was dissolved in DCM (50.00 mL) and dioxane (5.00 mL) and 2-aminothiophenol (4.79 g, 38.3 mmol) was added at 25° C. The resulting mixture was stirred for 10 h at 70° C. under nitrogen atmosphere. On completion, the reaction mixture was concentrated under vacuum. The residue was purified by reverse flash chromatography (Column, C18 silica gel; mobile phase, MeCN in water (0.1% NH4HCO3), 15% to 52% gradient in 30 min; detector, UV 220 nm) and concentrated to afford the title compound (13 g, 87% yield) as a yellow solid. 1H NMR (400 MHz, DMSO-d6) δ 8.08 (d, J=7.9 Hz, 1H), 7.99-7.95 (m, 1H), 7.91 (d, J=8.1 Hz, 1H), 7.53-7.50 (m, 1H), 7.44-7.41 (m, 1H), 4.93-4.90 (m, 1H), 3.60 (s, 3H), 2.52 (d, J=1.9 Hz, 1H), 2.50-2.47 (m, 1H), 2.39-2.26 (m, 1H), 2.12-1.99 (m, 1H), 1.43 (s, 9H); LC/MS (ESI, m/z): [(M+1)]+=351.4.

Step 2—4-(Benzo[d]thiazol-2-yl)-4-((tert-butoxycarbonyl)amino)butanoic acid. To a stirred solution of methyl 4-(benzo[d]thiazol-2-yl)-4-((tert-butoxycarbonyl)amino)butanoate (13.00 g, 37.10 mmol) and THF (100.00 mL) was added a solution of LiOH (4.42 g, 185 mmol) in H2O (50.00 mL) dropwise at rt. The resulting mixture was stirred for 2 h at rt. On completion, the reaction mixture was acidified to pH 4 with conc. HCl. The resulting mixture was extracted with CH2Cl2 (3×20 mL). The combined organic layers were washed with brine (2×20 mL), and dried over anhydrous Na2SO4. After filtration, the filtrate was concentrated under reduced pressure to afford the title compound (5 g, 36% yield) as a yellow solid. 1H NMR (400 MHz, DMSO-d6) δ 12.22 (s, 1H), 8.11-8.04 (m, 1H), 7.98-7.65 (m, 1H), 7.90 (d, J=8.0 Hz, 1H), 7.53-7.49 (m, 1H), 7.44-7.41 (m, 1H), 4.94-4.92 (m, 1H), 2.42-2.39 (m, 2H), 2.34-2.21 (m, 1H), 2.08-1.94 (m, 1H), 1.42 (s, 9H); LC/MS (ESI, m/z): [(M+1)]+=337.0.

Step 3—Tert-butyl (4-amino-1-(benzo[d]thiazol-2-yl)-4-oxobutyl)carbamate. To a stirred solution of (4S)-4-(1,3-benzothiazol-2-yl)-4-[(tert-butoxycarbonyl)amino]butanoic acid (5.60 g, 16.7 mmol) and NH4C1 (1.78 g, 0.033 mmol) in DMF (50.00 mL) were added TEA (9.26 mL, 0.067 mmol) and HATU (8.23 g, 0.022 mmol) in portions at 25° C. under nitrogen atmosphere. The resulting mixture was stirred for 2 h at 25° C. under nitrogen atmosphere. On completion, the reaction mixture was concentrated under reduced pressure. The crude product was purified by reverse phase flash (Column: Spherical C18 Column, 20-40 um, 120 g; Mobile Phase A: Water (5 mmol/L NH4HCO3), Mobile Phase B: ACN; Flow rate: 60 mL/min; Gradient: 30% B to 50% B in 25 min, 254 nm; the fractions containing the desired product were collected at 44% B) to afford the title compound (2.3 g, 41% yield) as a white solid. 1H NMR (400 MHz, DMSO-d6) δ 8.08 (d, J=7.9 Hz, 1H), 7.98-7.94 (m, 1H), 7.89 (d, J=7.8 Hz, 1H), 7.52-7.49 (m, 1H), 7.43-7.41 (m, 1H), 7.33-7.30 (m, 1H), 6.80 (s, 1H), 4.90-4.83 (m, 1H), 2.28-2.20 (m, 3H), 2.01-1.98 (m, 1H), 1.42 (s, 9H); LC/MS (ESI, m/z): [(M+1)]+=336.7.

Step 4—Tert-butyl N-[(1R)-1-(1,3-benzothiazol-2-yl)-3-carbamoylpropyl]carbamate and tert-butyl N-[(1S)-1-(1,3-benzothiazol-2-yl)-3-carbamoylpropyl]carbamate. The mixture tert-butyl N-[1-(1,3-benzothiazol-2-yl)-3-carbamoylpropyl]carbamate (2.30 g) was separated by Prep-Chiral-HPLC with the following conditions [Column: CHIRAL ART Amylose-C NEO, 3*25 cm, 5 um; Mobile Phase A:CO2, Mobile Phase B: MeOH (0.1% 2 M NH3-MeOH); Flow rate: 100 mL/min; Gradient: 40% B; 220 nm; RT1: 2.3; RT2: 2.5; Injection Volume: 1 ml; Number Of Runs: 40;] to afford tert-butyl N-[(1R)-1-(1,3-benzothiazol-2-yl)-3-carbamoylpropyl]carbamate (1 g, 44% yield, 1H NMR (400 MHz, Methanol-d4) δ 7.98-7.94 (m, 2H), 7.53-7.47 (m, 1H), 7.45-7.40 (m, 1H), 5.06-5.01 (m, 1H), 2.47-2.40 (m, 3H), 2.22-2.16 (m, 1H), 1.49 (s, 9H) (faster eluting isomer) as a white solid and tert-butyl N-[(1S)-1-(1,3-benzothiazol-2-yl)-3-carbamoylpropyl]carbamate (480 mg, 21% yield, 1HNMR (400 MHz, Methanol-d4) δ 8.00-7.94 (m, 2H), 7.54-7.49 (m, 1H), 7.46-7.41 (m, 1H), 5.07-5.02 (m, 1H), 2.47-2.41 (m, 3H), 2.24-2.13 (m, 1H), 1.49 (s, 9H) (slower eluting isomer) as a white solid. LC/MS (ESI, m/z): [(M+1)]+=336.1 for both isomers.

Step 5—(4S)-4-amino-4-(1,3-benzothiazol-2-yl)butanamide trifluoroacetate. To a stirred solution of tert-butyl N-[(1S)-1-(1,3-benzothiazol-2-yl)-3-carbamoylpropyl]carbamate (480.00 mg, 1.431 mmol) in DCM (5.00 mL) was added TFA (2.00 mL) dropwise at 25° C. under nitrogen atmosphere. The resulting mixture was stirred for 2 h at 25° C. under nitrogen atmosphere. The resulting mixture was concentrated under reduced pressure to afford the title compound (450.00 mg) as an off-white semi-solid. 1H NMR (300 MHz, Methanol-d4) δ 8.13-7.94 (m, 2H), 7.64-7.43 (m, 2H), 3.54-3.99 (m, 1H), 2.88-2.73 (m, 1H), 2.57-2.43 (m, 2H), 2.37-2.25 (m, 1H); LC/MS (ESI, m/z): [(M+1)]+=236.1.

### 4-Amino-4-methylpentanamide (Intermediate DX) (CAS #725233-37-4)

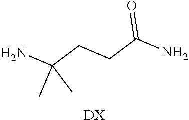

### 3-(5-amino-3-methyl-2-oxo-1,3-benzodiazol-1-yl)piperidine-2,6-dione (Intermediate DY)

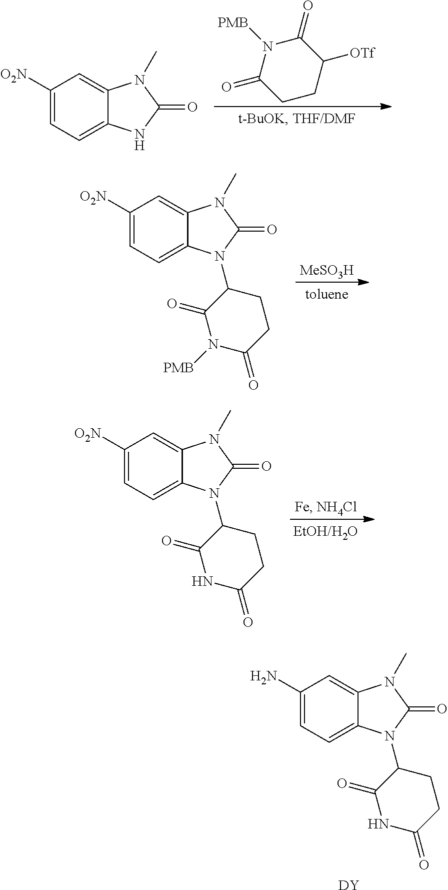

Step 1—1-[(4-methoxyphenyl)methyl]-3-(3-methyl-5-nitro-2-oxo-1,3-benzodiazol-1-yl)piperidine-2,6-dione. To a stirred mixture of 1-methyl-6-nitro-3H-1,3-benzodiazol-2-one (4.00 g, 20.7 mmol, CAS #206431-05-2) and t-BuOK (3.25 g, 29.0 mmol) in THF (200.00 mL) and DMF (50.00 mL) at rt. To the above mixture was added 1-[(4-methoxyphenyl)methyl]-2,6-dioxopiperidin-3-yl trifluoromethanesulfonate (8.69 g, 22.8 mmol, CAS #2304754-47-8) in portions at rt. The resulting mixture was stirred for an additional 16 h at rt. On completion, the reaction mixture was concentrated under reduced pressure. The resulting mixture was diluted with water (1000 mL). The precipitated solids were collected by filtration and washed with water (3×40 mL). The resulting solid was dried in an oven under reduced pressure to afford the title compound (8.3 g, 94% yield) as a green solid. ¹H NMR (300 MHz, DMSO-d6) δ 8.20-8.13 (m, 1H), 8.09-7.95 (m, 1H), 7.34-7.30 (m, 1H), 7.25-7.20 (m, 2H), 6.93-6.84 (m, 2H), 5.81-5.64 (m, 1H), 4.91-4.73 (m, 2H), 3.76 (s, 3H), 3.48 (s, 2H), 3.39-3.36 (m, 1H), 3.18-3.00 (m, 1H), 2.94-2.84 (m, 2H), 2.18-2.14 (m, 1H); LC/MS (ESI, m/z): [(M+H)]+=425.1.

Step 2—3-(3-methyl-5-nitro-2-oxo-1,3-benzodiazol-1-yl)piperidine-2,6-dione. To a stirred mixture of 1-[(4-methoxyphenyl)methyl]-3-(3-methyl-5-nitro-2-oxo-2,3-dihydro-1H-1,3-benzodiazol-1-yl)piperidine-2,6-dione (5 g, 12 mmol) in toluene (25 mL) was added methanesulfonic acid (25 mL, 390 mmol) at rt under nitrogen atmosphere. The resulting mixture was stirred for 2 h at 110° C. under nitrogen atmosphere. On completion, the reaction mixture was cooled to rt and was concentrated under reduced pressure. The resulting mixture was diluted with ice-water (300 mL). The precipitated solids were collected by filtration and washed with water (3×20 mL). The crude product was purified by reverse flash chromatography (Column, C18 silica gel; mobile phase, ACN in water, 18% to 38% gradient in 25 min; detector, UV 254 nm) to afford the title compound (1.7 g, 46% yield) as a light brown solid. 1H NMR (300 MHz, DMSO-d6) δ 11.10 (s, 1H), 8.18-7.94 (m, 2H), 7.40 (d, J=8.8 Hz, 1H), 5.54 (dd, J=12.7, 5.4 Hz, 1H), 3.19 (s, 3H), 3.00-2.78 (m, 1H), 2.81-2.61 (m, 2H), 2.14-2.08 (m, 1H); LC/MS (ESI, m/z): [(M+H)]+=305.1.

Step 3—3-(5-amino-3-methyl-2-oxo-1,3-benzodiazol-1-yl)piperidine-2,6-dione. To a stirred mixture of 3-(3-methyl-5-nitro-2-oxo-1,3-benzodiazol-1-yl)piperidine-2,6-dione (2.80 g, 9.20 mmol) and Fe (3.60 g, 64.5 mmol) in EtOH (30.00 mL) and water (5.00 mL) at rt under nitrogen atmosphere. Then NH₄Cl (4.92 g, 92.0 mmol) was added and the reaction mixture was stirred for 2 h at 75° C. under nitrogen atmosphere. On completion, the reaction mixture was cooled to rt and the mixture was concentrated under reduced pressure. The residue was purified by reverse phase flash chromatography (Column: Spherical C18, 20-40 um, 330 g; Mobile Phase A: Water (plus 5 mM NH4NO3); Mobile Phase B: ACN; Flow rate: 80 mL/min; Gradient: 5%-5% B, 10 min, 100% B-30% B gradient in 25 min; Detector: 220 nm; the fractions containing the desired product were collected at 20% B) and concentrated under reduced pressure to afford the title compound (1.1 g, 44% yield) as a white solid. ¹H NMR (400 MHz, DMSO-d6) δ 11.10 (s, 1H), 6.75 (d, J=8.4 Hz, 1H), 6.39 (d, J=2.1 Hz, 1H), 6.28 (dd, J=8.3, 2.1 Hz, 1H), 5.23 (dd, J=12.8, 5.3 Hz, 1H), 4.84 (s, 2H), 3.23 (s, 3H), 2.95-2.82 (m, 1H), 2.73-2.55 (m, 2H), 2.01-1.91 (m, 1H); LC/MS (ESI, m/z): [(M+H)]+=275.1.

### 1-(2-chloro-4-methanesulfonylphenyl)methanamine (Intermediate DZ)

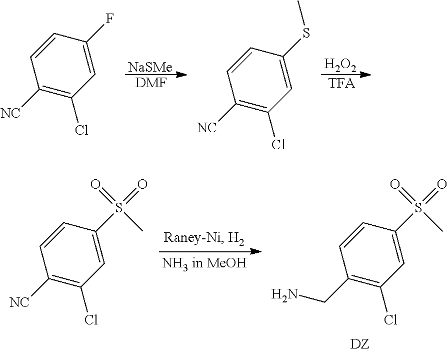

Step 1—2-chloro-4-(methylsulfanyl)benzonitrile. To a stirred solution of 2-chloro-4-fluorobenzonitrile (10.00 g, 64.28 mmol) in DMF (20.00 mL) was added Sodium methanethiolate (4.96 g, 70.7 mmol) in portions at rt under nitrogen atmosphere. The resulting mixture was stirred for 16 h at 80° C. under nitrogen atmosphere. On completion, the reaction mixture was cooled to rt and the mixture was extracted with EtOAc (3×200 mL) and water (1×200 mL). The combined organic layers were washed with brine (2×100 mL), and dried over anhydrous Na₂SO₄. After filtration, the filtrate was concentrated under reduced pressure. The crude product was purified by reverse phase flash chromatography (Column: WelFlash™ C18-I, 20-40 μm, 330 g; Eluent A: Water (plus 10 mmol/L NH₄HCO₃); Eluent B: ACN; Gradient: 35%-60% B in 30 min; Flow rate: 80 mL/min; Detector: 220/254 nm; desired fractions were collected at 55% B) and concentrated under reduced pressure to afford the title compound (6 g, 51% yield) as a white solid. 1H NMR (300 MHz, Chloroform-d) δ 7.54-7.49 (m, 1H), 7.29-7.26 (m, 1H), 7.18-7.12 (m, 1H), 2.52 (s, 3H); LC/MS (ESI, m/z): [M]+=183.0.

Step 2—2-chloro-4-methanesulfonylbenzonitrile. To a stirred solution of 2-chloro-4-(methylsulfanyl)benzonitrile (5.00 g, 27.2 mmol) in TFA (50.00 mL) was added H₂O₂ (50.00 mL) dropwise at 0° C. under nitrogen atmosphere. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 μm, 330 g; EluentA: Water (plus 10 mmol/LNH4HCO3); Eluent B: ACN; Gradient: 35%-55% B in 30 min; Flow rate: 80 mL/min; Detector: 220/254 nm; desired fractions were collected at 49% B) and concentrated under reduced pressure to afford the title compound (3.87 g, 66% yield) as a white solid. 1H NMR (300 MHz, Chloroform-d) δ 8.13-8.11 (m, 1H), 7.99-7.95 (m, 1H), 7.94-7.90 (m, 1H), 3.12 (s, 3H); LC/MS (ESI, m/z): [(M−H)]−=214.2.

Step 3—1-(2-chloro-4-methanesulfonylphenyl)methanamine. To a stirred solution of 2-chloro-4-methanesulfonylbenzonitrile (4.50 g, 20.9 mmol) in 7 M NH₃ (g) in MeOH (100.00 mL) was added Raney Ni (5.36 g, 62.6 mmol) dropwise at rt under nitrogen atmosphere. The resulting mixture was stirred for 16 h at rt under hydrogen atmosphere. On completion, the reaction mixture was filtered and the filter cake was washed with MeOH (3×50 mL). The filtrate was concentrated under reduced pressure. The residue was purified by reverse phase flash chromatography (Column: WelFlash™ C18-I, 20-40 m, 330 g; Eluent A: Water (plus 10 mmol/L NH₄HCO₃); Eluent B: ACN; Gradient: 15%-35% B in 30 min; Flow rate: 80 mL/min; Detector: 220/254 nm; desired fractions were collected at 20% B) and concentrated under reduced pressure to afford the title compound (2.9 g, 63% yield) as a light green solid. ¹H NMR (300 MHz, Methanol-d4) δ 7.98-7.87 (m, 2H), 7.79-7.73 (m, 1H), 3.99 (s, 2H), 3.18 (s, 3H); LC/MS (ESI, m/z): [(M−H)]−=218.0.

### 4-[1-(2,6-dioxopiperidin-3-yl)-3-methyl-2-oxo-1,3-benzodiazol-5-yl]butanal (Intermediate EA)

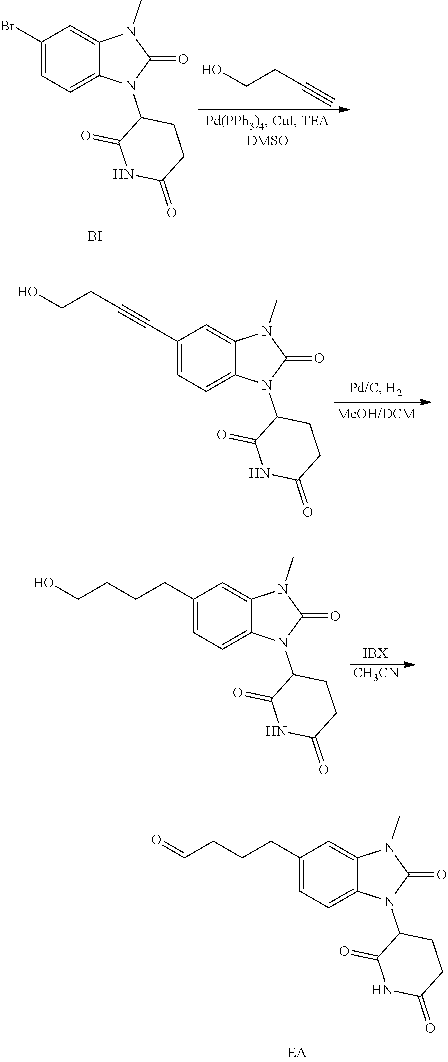

Step 1—3-[5-(4-hydroxybut-1-yn-1-yl)-3-methyl-2-oxo-1,3-benzodiazol-1-yl]piperidine-2,6-dione To a stirred mixture of 3-(5-bromo-3-methyl-2-oxo-1,3-benzodiazol-1-yl)piperidine-2,6-dione (6.00 g, 17.7 mmol, Intermediate BI) and 3-butyn-1-ol (3.73 g, 53.2 mmol) in TEA (15.00 mL)/DMSO (30.00 mL) was added CuI (337.92 mg, 1.774 mmol) and Pd(PPh₃)₄ (2.05 g, 1.77 mmol) at rt under nitrogen atmosphere. The mixture was allowed to react for 3 h at 90° C. under nitrogen atmosphere. On completion, the reaction mixture was cooled to rt. The mixture was then filtered, and the filter cake was washed with CH₂Cl₂ (2×10 mL). The filtrate was concentrated under reduced pressure. The residue was purified by reverse phase flash chromatography (Column: WelFlash™ C18-I, 20-40 ?m, 330 g; EluentA: Water (plus 10 mmol/L FA); Eluent B: ACN; Gradient: 18%-40% B in 20 min; Flow rate: 80 mL/min; Detector: 220/254 nm; desired fractions were collected at 26% B) and concentrated under reduced pressure to afford the title compound (4.5 g, 78% yield) as a white solid. ¹H NMR (400 MHz, DMSO-d6) δ 11.11 (s, 1H), 7.26-7.24 (m, 1H), 7.13-7.08 (m, 2H), 5.38 (dd, J=12.8, 5.3 Hz, 1H), 4.89-4.87 (m, 1H), 3.63-3.58 (m, 2H), 3.34 (s, 3H), 2.95-2.83 (m, 1H), 2.75-2.59 (m, 2H), 2.57-2.53 (m, 2H), 2.06-2.00 (m, 1H); LC/MS (ESI, m/z): [(M+H)]+=328.1.

Step 2—3-[5-(4-hydroxybutyl)-3-methyl-2-oxo-1,3-benzodiazol-1-yl]piperidine-2,6-dione. To a stirred solution of 3-[5-(4-hydroxybut-1-yn-1-yl)-3-methyl-2-oxo-1,3-benzodiazol-1-yl]piperidine-2,6-dione (1.50 g, 4.58 mmol) in MeOH (25.00 mL)/HOAc (2.00 mL, 34.9 mmol) was added Pd/C (487.66 mg, 0.458 mmol, 10 wt %) at rt under nitrogen atmosphere. The mixture was stirred for 4 h under hydrogen atmosphere at rt. On completion, the reaction mixture was filtered and the filter cake was washed with MeOH (3×20 mL). The filtrate was concentrated under reduced pressure to give the title compound (1.2 g, 75% yield) as a white solid. ¹H NMR (300 MHz, DMSO-d6) δ 11.09 (s, 1H), 7.10-6.97 (m, 2H), 6.90-6.87 (m, 1H), 5.35 (dd, J=12.8, 5.4 Hz, 1H), 4.40-4.36 (m, 1H), 3.34 (s, 3H), 2.95-2.87 (m, 1H), 2.82-2.57 (m, 4H), 2.10-1.93 (m, 1H), 1.69-1.56 (m, 2H), 1.49-1.44 (m, 2H); LC/MS (ESI, m/z): [(M+H)]+=332.2.

Step 3-4-[1-(2,6-dioxopiperidin-3-yl)-3-methyl-2-oxo-1,3-benzodiazol-5-yl]butanal. To a stirred mixture of IBX (1521.04 mg, 5.432 mmol) in ACN (30.00 mL) was added IBX (1521.04 mg, 5.432 mmol) in portions at rt under air atmosphere. The resulting mixture was stirred for 20 min at 80° C. under air atmosphere. On completion, the mixture was filtered and the filter cake was washed with acetonitrile (5×3 mL). The filtrate was concentrated under reduced pressure. The residue was purified by silica gel column chromatography, eluted with CH₂C1₂/MeOH (10:1), to afford the title compound (500 mg, 84% yield) as a yellow liquid. ¹H NMR (400 MHz, DMSO-d6) δ 11.09 (s, 1H), 9.68 (s, 1H), 7.07-6.99 (m, 2H), 6.89-6.85 (m, 1H), 5.35 (dd, J=12.7, 5.4 Hz, 1H), 3.33 (s, 3H), 2.94-2.87 (m, 1H), 2.74-2.70 (m, 1H), 2.66-2.58 (m, 3H), 2.48-2.44 (m, 2H), 2.02-1.98 (m, 1H), 1.91-1.79 (m, 2H).

### Example 1. HiBiT Assay Protocol

Compound preparation and Cell seeding: The transfected A549 cells were harvested from dish into cell culture medium and cell numbers counted. Cells were diluted with culture medium to the desired density and 30 μL of cell suspension (about 2000 cells/well) were added into each well of a 384-well cell culture plate as designated and transferred into 37° C. 5% CO₂ incubator for 24 h. Compounds were dissolved to 10 mM stock solution and 12 μL of the stock solution was transferred to a 384 LDV-plate. 3 fold, 10-point dilution was performed by transferring 4 μL compound into 8 μL DMSO using a TECAN (EVO200) liquid handler. 30 μl of diluted compound from compound source plate was transferred into the cell plate as designated by using Echo550 and transferred into 37° C. 5% CO₂ incubator for 24 h.

Detection: Plates were removed from incubators and equilibrated at room temperature for 15 minutes. Nano-Glo Hibit Lytic Detection reagent (Promega Cat #N3040) was thawed and equilibrated to room temperature before the experiment. 30 μL of Nano-Glo Hibit Lytic Detection reagent was added into each well to be detected. The plates were held at room temperature for 10 min followed by reading on EnSpire.

Data analysis: The remaining activity was calculated following the formula: Remaining Activity (%)=100%×(Lumsample−LumNC)/(LumPC−LumNC). Calculate the IC₅₀ by fitting the curve using Xlfit (v5.3.1.3), equation 201: fit=(A+((B−A)/(1+((x/C)\hat{ }D)))); A: Botton; B: Top; C: IC₅₀; and D: Slope.

STAT3 HiBiT degradation results for compounds of the invention are presented in Table 2. The letter codes for STAT3 DC₅₀ include: A (<0.01 μM), B (0.01-0.1 μM), C (0.1-1.0 μM), and D (>1.0 μM).

TABLE 2
STAT3 HiBiT Degradation Results

| | STAT3 HiBIT A549 |
|---|---|
| | degradation 24 h: |
| | Average external-Abs |
| I-# | DC50 (μM) |
| I-1 | A |
| I-2 | A |
| I-3 | A |
| I-4 | A |
| I-5 | A |
| I-6 | B |
| I-7 | A |
| I-8 | A |
| I-9 | A |
| I-10 | A |
| I-11 | B |
| I-12 | B |
| I-13 | B |
| I-14 | C |
| I-15 | B |
| I-20 | D |
| I-21 | D |
| I-22 | D |
| I-23 | D |
| I-24 | C |
| I-25 | D |
| I-26 | D |
| I-27 | D |
| I-28 | C |
| I-29 | D |
| I-30 | B |
| I-31 | D |
| I-32 | D |
| I-33 | B |
| I-34 | B |
| I-35 | B |
| I-36 | B |
| I-37 | B |
| I-38 | C |
| I-39 | B |
| I-40 | C |
| I-41 | C |
| I-42 | D |
| I-43 | D |
| I-44 | B |
| I-45 | B |
| I-46 | B |
| I-47 | B |
| I-48 | B |
| I-49 | B |
| I-50 | C |
| I-51 | B |
| I-52 | C |
| I-53 | A |
| I-54 | A |
| I-55 | B |
| I-56 | C |
| I-57 | B |
| I-58 | B |
| I-59 | B |
| I-60 | B |
| I-61 | B |
| I-62 | B |
| I-63 | A |
| I-64 | D |
| I-65 | D |
| I-66 | A |
| I-67 | C |
| I-68 | B |
| I-69 | A |
| I-70 | B |
| I-71 | B |
| I-72 | B |
| I-73 | B |
| I-74 | B |
| I-75 | D |
| I-76 | B |
| I-77 | A |
| I-78 | A |
| I-79 | A |
| I-80 | A |
| I-81 | B |
| I-82 | D |
| I-83 | D |
| I-84 | D |
| I-86 | D |
| I-87 | D |
| I-88 | D |
| I-89 | D |
| I-96 | D |
| I-97 | D |
| I-98 | D |

### Example 2 (Method 1). Synthesis of 2-[[(3S,6S,10aS)-3-I[(2S)-4-carbamoyl-1-[2-chloro-3-(4-II(2S)-1-[(2S,4R)-4-hydroxy-2-I[[(1S)-1-[4-(4-methyl-1,3-thiazol-5-yl)phenyl] ethyl] carbamoyl] pyrrolidin-1-yl]-3,3-dimethyl-1-oxobutan-2-yl] carbamoyl] butyl)phenoxy] butan-2-yl] carbamoyl]-5-oxo-octahydro-1H-pyrrolo[1,2-a]azocin-6-yl]carbamoyl]-1H-indole-5-carbonylphosphonic acid (I-1)

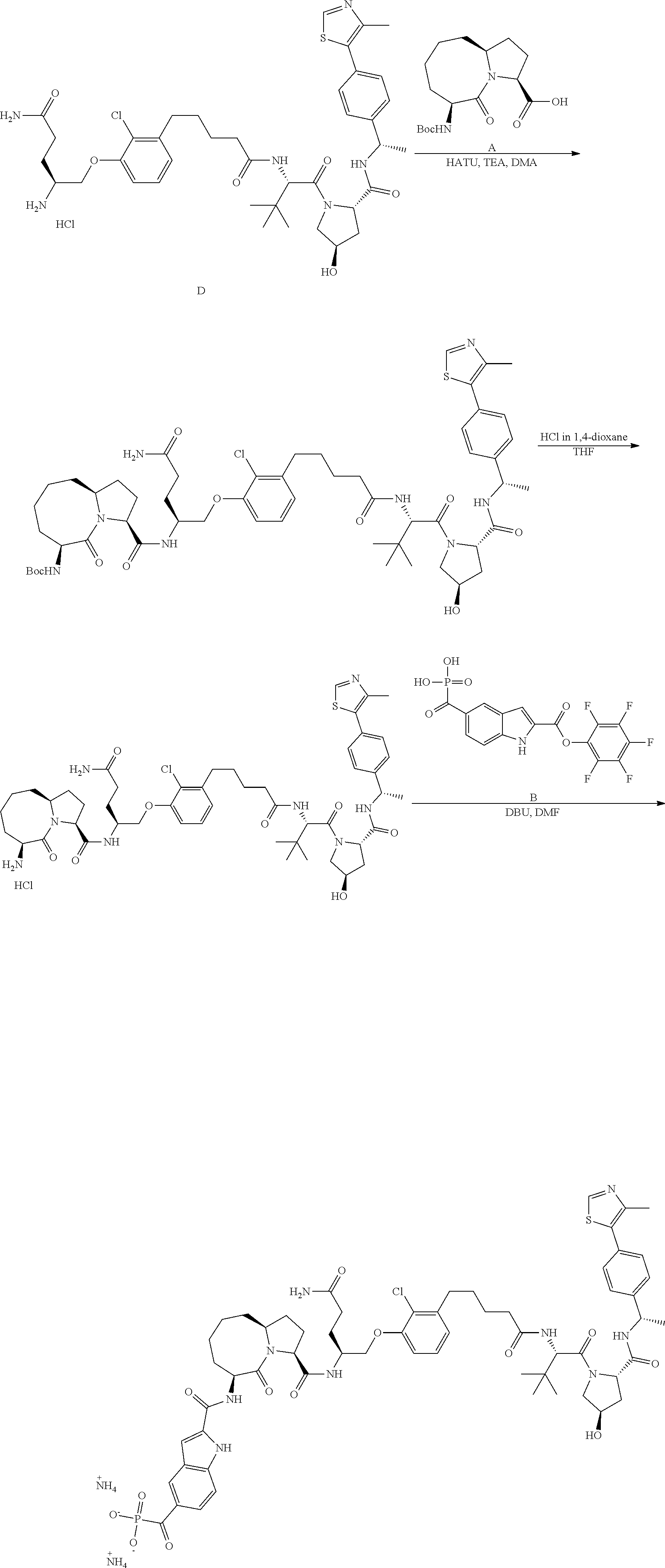

Step 1—tert-butyl N-[(3S,6S,10aS)-3-[[(2S)-4-carbamoyl-1-[2-chloro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamoyl]-5-oxo-octahydro-1H-pyrrolo[1,2-a]azocin-6-yl]carbamate. To a stirred mixture of (3S,6S,10aS)-6-[(tert-butoxycarbonyl)amino]-5-oxo-octahydro-1H-pyrrolo[1,2-a]azocine-3-carboxylic acid (128.80 mg, 0.40 mmol, Intermediate A) and HATU (150.04 mg, 0.40 mmol) in DMA (5 mL) were added TEA (99.83 mg, 1.0 mmol) dropwise and (2S,4R)-1-[(2S)-2-(5-[3-[(2S)-2-amino-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (265.00 mg, 0.329 mmol, Intermediate D) in portions at rt under nitrogen atmosphere. The resulting mixture was stirred for 16 h at rt under nitrogen atmosphere. On completion, the residue was purified by reverse flash chromatography (column, C¹⁸ silica gel; mobile phase, MeCN in water (10 mmol/L NH₄HCO₃), 40% to 60% gradient in 25 min; detector, UV 254 nm) to afford the title compound (289.3 mg, 82% yield) as a white solid; ¹H NMR (400 MHz, DMSO-d₆) δ 8.99 (s, 1H), 8.38 (d, J=7.8 Hz, 1H), 8.00 (d, J=8.3 Hz, 1H), 7.84 (d, J=9.2 Hz, 1H), 7.46-7.42 (m, 2H), 7.45-7.29 (m, 2H), 7.22-7.18 (m, 1H), 7.16-7.12 (m, 1H), 6.99-6.96 (m, 1H), 6.95-6.81 (m, 2H), 6.72 (s, 1H), 5.10 (d, J=3.3 Hz, 1H), 4.94-4.90 (m, 1H), 4.52 (d, J=9.3 Hz, 1H), 4.45-4.41 (m, 1H), 4.29 (s, 2H), 4.19-4.17 (m, 1H), 4.11-4.08 (m, 1H), 4.00-3.97 (m, 1H), 3.94-3.85 (m, 1H), 3.67-3.55 (m, 2H), 2.70-2.66 (m, 2H), 2.46 (s, 3H), 2.35-2.27 (m, 1H), 2.25-2.11 (m, 3H), 2.08-1.97 (m, 3H), 1.94-1.74 (m, 4H), 1.74-1.60 (m, 2H), 1.59-1.50 (m, 9H), 1.43-1.31 (m, 12H), 1.33-1.05 (m, 3H), 0.94 (s, 9H); LC/MS (ESI, m/z): [(M+1)]⁺=1077.5.

Step 2—(2S,4R)-1-[(2S)-2-(5-[3-[(2S)-2-[[(3S,6S,10aS)-6-amino-5-oxo-octahydro-1H-pyrrolo[1,2-a]azocin-3-yl]formamido]-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(3S,6S,10aS)-3-[[(2S)-4-carbamoyl-1-[2-chloro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamoyl]-5-oxo-octahydro-1H-pyrrolo[1,2-a]azocin-6-yl]carbamate (289.30 mg, 0.27 mmol) in THF (5.00 mL) was added HCl (gas) in 1,4-dioxane (5 mL) dropwise at rt under nitrogen atmosphere. The resulting mixture was stirred for 2 h at rt under nitrogen atmosphere. On completion, the reaction mixture was concentrated under reduced pressure to the title compound (263 mg, 97% yield) as a light yellow solid. ¹H NMR (400 MHz, DMSO-d₆) δ 9.18 (s, 1H), 8.43 (d, J=7.7 Hz, 1H), 8.24-8.20 (m, 3H), 8.17 (d, J=8.2 Hz, 1H), 7.84 (d, J=9.2 Hz, 1H), 7.48-7.44 (m, 2H), 7.42-7.38 (m, 2H), 7.22-7.18 (m, 1H), 6.98 (d, J=8.2 Hz, 1H), 6.91 (d, J=7.6 Hz, 1H), 6.78-6.75 (m, 1H), 4.94-4.90 (m, 1H), 4.51 (d, J=9.2 Hz, 1H), 4.45-4.42 (m, 1H), 4.36-4.32 (m, 2H), 4.30-4.17 (m, 3H), 4.15-3.98 (m, 2H), 3.93-3.89 (m, 1H), 3.65-3.61 (m, 2H), 3.60-3.57 (m, 2H), 3.43-3.36 (m, 1H), 2.70-2.66 (m, 2H), 2.48 (s, 3H), 2.38-2.09 (m, 4H), 2.08-1.84 (m, 4H), 1.84-1.64 (m, 5H), 1.63-1.45 (m, 8H), 1.38 (d, J=7.0 Hz, 3H), 0.93 (s, 9H); LC/MS (ESI, m z): [(M+1)]⁺=977.4.

Step 3—Diammonium 2-[[(3S,6S,10aS)-3-[[(2S)-4-carbamoyl-1-[2-chloro-3-(4-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]butyl)phenoxy]butan-2-yl]carbamoyl]-5-oxo-octahydro-1H-pyrrolo[1,2-a]azocin-6-yl]carbamoyl]-1H-indole-5-carbonylphosphonate. To a stirred solution of (2S,4R)-1-[(2S)-2-(5-[3-[(2S)-2-[[(3S,6S,10aS)-6-amino-5-oxo-octahydro-1H-pyrrolo[1,2-a]azocin-3-yl]formamido]-4-carbamoylbutoxy]-2-chlorophenyl]pentanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (263.00 mg, 0.26 mmol) and 2-(2,3,4,5,6-pentafluorophenoxycarbonyl)-1H-indole-5-carbonylphosphonic acid (225.73 mg, 0.52 mmol, Intermediate B) in NMP (3.00 mL) was added DBU (197.40 mg, 1.297 mmol) dropwise at rt under nitrogen atmosphere. The resulting mixture was stirred for 16 h at rt under nitrogen atmosphere. On completion, the residue was purified by reverse flash chromatography (column, C¹⁸ silica gel; mobile phase, MeCN in water (10 mmol/L NH₄HCO₃), 20% to 40% gradient in 25 min; detector, UV 220 nm) to afford the title compound (117.4 mg, 36% yield) as a white solid; ¹H NMR (400 MHz, DMSO-d₆) δ 11.92-11.89 (m, 1H), 8.99 (s, 1H), 8.89-8.87 (m, 1H), 8.73-8.63 (m, 1H), 8.41 (d, J=7.8 Hz, 1H), 8.07-8.04 (m, 1H), 7.97-7.93 (m, 1H), 7.85 (d, J=9.3 Hz, 1H), 7.47-7.28 (m, 6H), 7.24-7.15 (m, 2H), 7.01-6.95 (m, 1H), 6.92-6.88 (m, 1H), 6.79 (s, 1H), 5.03-4.99 (m, 1H), 4.94-4.90 (m, 1H), 4.52 (d, J=9.3 Hz, 1H), 4.46-4.42 (m, 1H), 4.35-4.26 (m, 3H), 4.14-4.07 (m, 1H), 4.05-3.98 (m, 1H), 3.93-3.89 (m, 1H), 3.66-3.58 (m, 2H), 2.71-2.66 (m, 2H), 2.61-2.57 (m, 1H), 2.46 (s, 3H), 2.36-2.28 (m, 1H), 2.25-2.12 (m, 3H), 2.11-1.96 (m, 4H), 1.95-1.84 (m, 6H), 1.84-1.75 (m, 2H), 1.74-1.62 (m, 3H), 1.62-1.43 (m, 7H), 1.38 (d, J=7.0 Hz, 3H), 0.94 (s, 9H); LC/MS (ESI, m z): [(M+1)]⁺=1229.5.

TABLE 3
Compounds synthesized via Method 1, where the corresponding amines and acids were
coupled in Step 1.

| | | | LC/MS | |
|---|---|---|---|---|
| | Step 1 | Step 1 | (ESI, m/z) | |
| I-#ª | Amine | Acid | (M + H)⁺ | 1H NMR (400 MHZ, DMSO-d6) δ |
| I-2 | F | A | 1212.6^{b} | 11.92 (s, 1H), 8.99 (s, 1H), 8.89-8.85 (m, 1H), 8.73-8.68 (m, |
| | | | | 1H), 8.41 (d, J = 7.8 Hz, 1H), 8.08-8.02 (m, 1H), 8.00-7.86 |
| | | | | (m, 2H), 7.48-7.34 (m, 6H), 7.25-7.17 (m, 2H), 7.02-6.96 |
| | | | | (m, 1H), 6.91-6.87 (m, 1H), 6.79 (s, 1H), 5.05-4.98 (m, 1H), |
| | | | | 4.96-4.88 (m, 1H), 4.54 (d, J = 9.3 Hz, 1H), 4.44 (t, J = 8.0 |
| | | | | Hz, 1H), 4.36-4.27 (m, 3H), 4.15-4.07 (m, 2H), 4.05-3.97 |
| | | | | (m, 2H), 3.96-3.87 (m, 2H), 3.64-3.60 (m, 2H), 2.72-2.63 |
| | | | | (m, 2H), 2.46 (s, 3H), 2.36-2.13 (m, 5H), 2.10-1.98 (m, 4H), |
| | | | | 1.91-1.74 (m, 8H), 1.73-1.62 (m, 3H), 1.61-1.46 (m, 2H), |
| | | | | 1.38 (s, 3H), 0.95 (s, 9H) |
| I-7 | AA | A | 1212.6 | 11.83 (s, 1H), 8.99-8.96 (m, 1H), 8.90-8.87 (m, 1H), 8.71- |
| | | | | 8.62 (m, 1H), 8.44-8.36 (m, 1H), 8.06-7.84 (m, 3H), 7.47- |
| | | | | 7.36 (m, 6H), 7.22-7.17 (m, 1H), 6.88-6.73 (m, 2H), 6.64- |
| | | | | 6.57 (m, 1H), 5.07-4.88 (m, 2H), 4.56-4.40 (m, 2H), 4.36- |
| | | | | 4.26 (m, 3H), 4.11-3.82 (m, 4H), 3.67-3.58 (m, 2H), 3.51- |
| | | | | 3.40 (m, 2H), 3.06-2.91 (m, 1H), 2.46 (s, 3H), 2.33-2.13 (m, |
| | | | | 8H), 2.10-1.95 (m, 4H), 1.90-1.66 (m, 12H), 1.62-1.54 (m, |
| | | | | 2H), 1.38 (d, J = 7.0 Hz, 3H), 0.95 (s, 9H) |
| I-8 | BX | AF | 1233.5 | 11.85 (s, 1H), 8.99 (s, 1H), 8.88 (s, 1H), 8.68-8.64 (m, 1H), |
| | | | | 8.41-8.37 (m, 1H), 8.05-8.01 (m, 1H), 7.99-7.85 (m, 2H), |
| | | | | 7.48-7.19 (m, 9H), 7.01-6.97 (m, 1H), 6.80-6.76 (m, 2H), |
| | | | | 5.01-4.88 (m, 1H), 4.56-4.52 (m, 1H), 4.46-4.42 (m, 1H), |
| | | | | 4.32-4.29 (m, 3H), 4.09-4.5 (m, 2H), 3.96-3.91 (m, 1H), |
| | | | | 3.64-3.60 (m, 2H), 3.44-3.40 (m, 2H), 2.70-2.65 (m, 2H), |
| | | | | 2.47 (s, 3H), 2.20-2.18 (m, 4H), 2.09-1.99 (m, 4H), 1.90- |
| | | | | 1.74 (m, 9H), 1.71-1.68 (m, 3H), 1.64-1.45 (m, 2H), |
| | | | | 1.401.36 (m, 3H), 0.95 (s, 9H) |
| I-10 | CT | A | 1240.5 | 8.97 (s, 1H), 8.88 (s, 1H), 8.82 (s, 1H), 8.69-8.65 (m, 1H), |
| | | | | 8.59-8.55 (m, 1H), 8.05-8.01 (m, 1H), 7.99-7.91 (m, 2H), |
| | | | | 7.43-7.40 (m, 1H), 7.37 (s, 1H), 7.35-7.24 (m, 4H), 7.24- |
| | | | | 7.15 (m, 2H), 6.99-6.95 (m, 1H), 6.93-6.89 (m, 1H), 6.78- |
| | | | | 6.74 (m, 1H), 5.03-4.99 (m, 1H), 4.57-4.53 (m, 1H), 4.42- |
| | | | | 4.38 (m, 1H), 4.37-4.28 (m, 3H), 4.12-4.08 (m, 1H), 3.95- |
| | | | | 3.87 (m, 1H), 3.66-3.62 (m, 2H), 2.71-2.67 (m, 2H), 2.44 (s, |
| | | | | 3H), 2.36-2.32 (m, 1H), 2.22-2.18 (m, 3H), 2.10-1.96 (m, |
| | | | | 3H), 1.94-1.81 (m, 10H), 1.73-1.64 (m, 3H), 1.62-1.48 (m, |
| | | | | 8H), 1.25-1.21 (m, 4H), 0.94 (s, 9H) |
| I-16 | D | BM | 1231.4 | 11.88 (s, 1H), 8.99 (s, 1H), 8.89-8.85 (m, 1H), 8.76 (d, J = |
| | | | | 7.9 Hz, 1H), 8.42 (d, J = 7.9 Hz, 1H), 7.98-7.93 (m, 1H), |
| | | | | 7.92-7.81 (m, 2H), 7.48-7.30 (m, 6H), 7.25-7.16 (m, 2H), |
| | | | | 7.03-6.95 (m, 1H), 6.91 (d, J = 7.5 Hz, 1H), 6.83-6.78 (m, |
| | | | | 1H), 4.97-4.88 (m, 1H), 4.87-4.83 (m, 1H), 4.53 (d, J = 9.3 |
| | | | | Hz, 1H), 4.44 (t, J = 8.0 Hz, 1H), 4.37-4.30 (m, 1H), 4.29- |
| | | | | 4.25 (m, 1H), 4.09-4.05 (m, 1H), 4.00-3.89 (m, 2H), 3.88- |
| | | | | 3.79 (m, 1H), 3.66-3.55 (m, 4H), 3.50-3.30 (m, 1H), 2.71- |
| | | | | 2.67 (m, 3H), 2.46 (s, 3H), 2.33-2.29 (m, 1H), 2.22-2.13 (m, |
| | | | | 3H), 2.07-2.03 (m, 2H), 1.95-1.78 (m, 3H), 1.77-1.66 (m, |
| | | | | 4H), 1.56-1.52 (m, 6H), 1.38 (d, J = 7.0 Hz, 3H), 0.96-0.90 |
| | | | | (m, 15H) |
| I-17 | F | BM | 1216.5 | 11.82 (s, 1H), 10.07 (s, 1H), 8.99 (s, 1H), 8.90 (s, 1H), 8.73 |
| | | | | (d, J = 8.0 Hz, 1H), 8.41 (d, J = 7.7 Hz, 1H), 7.96-7.83 (m, |
| | | | | 3H), 7.46-7.34 (m, 7H), 7.25-7.18 (m, 2H), 6.99 (d, J = |
| | | | | 8.2 Hz, 1H), 6.89 (d, J = 7.7 Hz, 1H), 6.79 (s, 1H), 4.97- |
| | | | | 4.80 (m, 2H), 4.54 (d, J = 9.1 Hz, 1H), 4.43 (t, J = 8.2 Hz, |
| | | | | 1H), 4.36-4.27 (m, 1H), 4.06 (s, 1H), 4.01-3.89 (m, 2H), |
| | | | | 3.87-3.76 (m, 1H), 3.63-3.60 (m, 2H), 3.55-3.50 (m, 1H), |
| | | | | 3.48-3.43 (m, 2H), 3.26-3.18 (m, 2H), 2.71-2.61 (m, 4H), |
| | | | | 2.46 (s, 3H), 2.35-2.28 (m, 1H), 2.22-2.17 (m, 2H), 2.07- |
| | | | | 1.98 (m, 2H), 1.92-1.86 (m, 3H), 1.81-1.73 (m, 4H), 1.68- |
| | | | | 1.63( m, 1H), 1.62-1.54 (m, 3H), 1.37 (d, J = 6.9 Hz, 3H), |
| | | | | 0.95 (s, 9H), 0.93-0.91 (m, 3H) |
| I-18^{b} | BN | BM | 1213.1 | 11.84 (s, 1H), 8.98 (s, 1H), 8.87 (s, 1H), 8.73 (d, J = 7.6 Hz, |
| | | | | 1H), 8.42-8.37 (m, 1H), 7.97-7.93 (m, 1H), 7.88 (d, J = 8.4 |
| | | | | Hz, 1H), 7.82 (d, J = 9.3 Hz, 1H), 7.47-7.29 (m, 6H), 7.20 |
| | | | | (s, 1H), 7.05-6.94 (m, 2H), 6.86-6.75 (m, 2H), 4.94-4.90 (m, |
| | | | | 1H), 4.89-4.80 (m, 1H), 4.51 (d, J = 9.3 Hz, 1H), 4.43 (t, J = |
| | | | | 8.0 Hz, 1H), 4.35-4.29 (m, 1H), 4.29-4.25 (m, 1H), 4.06- |
| | | | | 3.98 (m, 1H), 4.00-3.87 (m, 2H), 3.84-3.77 (m, 1H), 3.65- |
| | | | | 3.57 (m, 3H), 2.58-2.55 (m, 3H), 2.45 (s, 3H), 2.33-2.25 (m, |
| | | | | 1H), 2.22-2.10 (m, 3H), 2.07-1.97 (m, 3H), 1.93-1.73 (m, |
| | | | | 4H), 1.72-1.62 (m, 3H), 1.61-1.44 (m, 7H), 1.37 (d, J = 7.0 |
| | | | | Hz, 3H), 0.94-0.90 (m, 15H) |
| I-19 | BF | BM | 1228.7 | (Methanol-d₄) δ 8.99-8.86 (m, 2H), 8.58 (d, J = 7.6 Hz, 1H), |
| | | | | 8.18 (d, J = 8.7 Hz, 1H), 8.13-8.06 (m, 1H), 7.52-7.39 (m, |
| | | | | 5H), 7.00-6.94 (m, 1H), 6.85-6.79 (m, 1H), 5.06-4.91 (m, |
| | | | | 2H), 4.67-4.56 (m, 2H), 4.46-4.42 (m, 2H), 4.29-4.20 (m, |
| | | | | 1H), 4.14-3.95 (m, 3H), 3.90 (d, J = 11.0 Hz, 1H), 3.79-3.69 |
| | | | | (m, 2H), 3.63-3.34 (m, 4H), 2.67-2.65 (m, 2H), 2.50 (s, 3H), |
| | | | | 2.42-1.95 (m, 10H), 1.90-1.78 (m, 3H), 1.74-1.59 (m, 4H), |
| | | | | 1.54-1.48 (m, 3H), 1.42-1.27 (m, 2H), 1.09-0.99 (m, 13H) |
| I-32 | CC | A | 1015.5 | 11.99 (s, 1H), 11.08 (s, 1H), 8.86-8.60 (m, 2H), 8.15-7.85 |
| | | | | (m, 2H), 7.58-7.35 (m, 2H), 7.07-6.80 (m, 7H), 6.80-6.68 |
| | | | | (m, 1H), 5.39-5.25 (m, 1H), 5.12-4.93 (m, 1H), 4.41-4.28 |
| | | | | (m, 3H), 4.16-4.01 (m, 6H), 3.33 (s, 3H), 2.97-2.81 (m, 1H), |
| | | | | 2.80-2.54 (m, 5H), 2.23-2.19 (m, 2H), 2.15-1.82 (m, 9H), |
| | | | | 1.82-1.54 (m, 8H), 1.40-1.22 (m, 1H) |
| I-42 | AD | H | 1258.9 | 12.06-11.88 (m, 1H), 9.00 (s, 1H), 8.91-8.77 (m, 1H), 8.50 |
| | | | | (d, J = 7.0 Hz, 1H), 8.34 (d, J = 7.7 Hz, 1H), 8.12-8.00 (m, |
| | | | | 1H), 8.00-7.87 (m, 2H), 7.49-7.36 (m, 6H), 7.33-7.17 (m, |
| | | | | 4H), 7.05-6.96 (m, 1H), 6.90 (d, J = 7.5 Hz, 1H), 6.80-6.69 |
| | | | | (m, 1H), 5.38-5.34 (m, 1H), 5.06-5.02 (m, 1H), 4.93 (t, J = |
| | | | | 7.2 Hz, 1H), 4.47 (d, J = 8.7 Hz, 1H), 4.44-4.29 (m, 2H), |
| | | | | 4.25-4.19 (m, 2H), 4.04-379 (m, 5H), 3.72-3.68 (m, 2H), |
| | | | | 3.43-3.36 (m, 2H), 2.77-2.58 (m, 2H), 2.48 (s, 3H), 2.47- |
| | | | | 2.45 (m, 1H), 2.36-2.05 (m, 11H), 2.00-1.62 (m, 9H), 1.39 |
| | | | | (d, J = 7.0 Hz, 3H), 0.97 (s, 9H) |
| I-43 | AC | H | 1272.0 | 12.06-11.85 (m, 1H), 9.00 (s, 1H), 8.94-8.80 (m, 1H), 8.50 |
| | | | | (d, J = 6.9 Hz, 1H), 8.34 (d, J = 7.6 Hz, 1H), 8.20-7.94 (m, |
| | | | | 2H), 7.86 (d, J = 8.7 Hz, 1H), 7.49-7.33 (m, 6H), 7.30-7.12 |
| | | | | (m, 4H), 7.02-6.97 (m, 1H), 6.91 (dd, J = 7.7, 1.5 Hz, 1H), |
| | | | | 6.80-6.71(m, 1H), 5.36-5.32 (m, 1H), 5.06-5.02 (m, 1H), |
| | | | | 4.96-4.90 (m, 1H), 4.56-4.26 (m, 4H), 4.22-4.19 (m, 1H), |
| | | | | 4.12-3.81 (m, 5H), 3.74-3.69 (m, 2H), 3.65-3.21(m, 4H), |
| | | | | 2.75-2.68 (m, 2H), 2.47 (s, 3H), 2.36-2.00 (m, 8H), 1.91- |
| | | | | 1.83 (m, 3H), 1.75-1.62 (m, 4H), 1.55-1.51 (m, 4H), 1.39 (d, |
| | | | | J = 7.0 Hz, 3H), 0.96 (s, 9H) |
| I-63 | I | R | 1246.3 | 12.07 (s, 1H), 9.02-8.99 (m, 2H), 8.86 (s, 1H), 8.60-8.51 (m, |
| | | | | 1H), 8.17-8.13 (m, 1H), 7.98 (d, J = 8.8 Hz, 1H), 7.57-7.36 |
| | | | | (m, 6H), 7.25-7.21 (m, 2H), 7.15-7.06 (m, 2H), 7.05-6.98 |
| | | | | (m, 2H), 6.97-6.93 (m, 1H), 6.77 (s, 1H), 6.13 (s, 1H), 5.10 |
| | | | | (d, J = 10.8 Hz, 1H), 4.70-4.67 (m, 1H), 4.45-4.31 (m, 4H), |
| | | | | 4.22-4.18 (m, 1H), 4.11-3.94 (m, 4H), 3.80-3.76 (m, 1H), |
| | | | | 3.70-3.67 (m, 1H), 3.55-3.31 (m, 3H), 3.21-3.14 (m, 3H), |
| | | | | 2.98-2.96 (m, 1H), 2.89-2.76 (m, 2H), 2.46 (d, J = 10.2 Hz, |
| | | | | 3H), 2.36-2.23 (m, 4H), 2.22-2.11 (m, 2H), 2.08-2.00 (m, |
| | | | | 4H), 1.99-1.83 (m, 1H), 1.77-1.69 (m, 1H), 0.97 (d, J = 6.5 |
| | | | | Hz, 3H), 0.82 (d, J = 6.6 Hz, 3H) |
| I-69 | I | H | 1269.4 | 12.51-11.56 (m, 1H), 9.07-8.97 (m, 1H), 8.91 (s, 1H), 8.61- |
| | | | | 8.45 (m, 2H), 8.11-8.07 (m, 1H), 7.97 (d, J = 8.9 Hz, 1H), |
| | | | | 7.49-7.33 (m, 6H), 7.31-7.17 (m, 2H), 7.06-7.02 (m, 1H), |
| | | | | 6.94 (d, J = 7.6 Hz, 1H), 6.81 (s, 1H), 6.13-6.11 (m, 1H), |
| | | | | 5.07-4.99 (m, 1H), 4.45-4.33 (m, 4H), 4.32-4.24 (m, 1H), |
| | | | | 4.24-4.13 (m, 2H), 4.16-3.99 (m, 2H), 3.98-3.83 (m, 1H), |
| | | | | 3.82-3.64 (m, 4H), 3.53-3.29 (m, 4H), 2.88-2.78 (m, 3H), |
| | | | | 2.47 (s, 3H), 2.35-2.18 (m, 5H), 2.18-2.10 (m, 3H), 2.09- |
| | | | | 1.97 (m, 4H), 1.97-1.79 (m, 4H), 1.79-1.70 (m, 4H), 0.96 (d, |
| | | | | J = 6.5 Hz, 3H), 0.82 (d, J = 6.6 Hz, 3H) |
| I-73^{c} | BZ | BY | 1168.1 | 11.09 (s, 1H), 8.82 (s, 2H), 8.61-8.43 (m, 2H), 8.37-8.31 (m, |
| | | | | 1H), 8.01-7.94 (m, 2H), 7.89-7.84 (m, 2H), 7.60-7.38 (m, |
| | | | | 4H), 7.06-6.95 (m, 2H), 6.94-6.76 (m, 2H), 5.35-5.31 (m, |
| | | | | 1H), 5.04-4.98 (m, 1H), 4.48-4.42 (m, 1H), 4.45-4.38 (m, |
| | | | | 2H), 4.25-4.21 (m, 2H), 3.59-3.48 (m, 3H), 3.19-3.09 (m, |
| | | | | 7H), 2.88-2.81 (m, 4H), 2.66-2.61 (m, 2H), 2.60-2.51 (m, |
| | | | | 2H), 2.14-2.11 (m, 4H), 2.09-1.96 (m, 1H), 1.79-1.72 (m, |
| | | | | 10H), 1.51-1.44 (m, 3H), 1.35-1.00 (m, 2H) |
| I-77 | BX | H | 1275.4 | 11.90 (s, 1H), 8.96-8.90 (m, 2H), 8.54-8.16 (m, 1H), 8.10- |
| | | | | 7.87 (m, 2H), 7.46-7.39 (m, 6H), 7.31-6.95 (m, 3H), 6.88- |
| | | | | 6.61 (m, 2H), 5.09-4.89 (m, 2H), 4.57-4.53 (m, 1H), 4.43- |
| | | | | 4.39 (m, 2H), 4.31-4.27 (m, 2H), 4.10-4.06 (m, 2H), 4.00- |
| | | | | 3.82 (m, 3H), 3.67-3.40 (m, 6H), 2.72-2.65 (m, 6H), 2.47 (s, |
| | | | | 3H), 2.37-2.05 (m, 9H), 2.95-1.78 (m, 8H), 1.39 (d, J = 6.8 |
| | | | | Hz, 3H), 0.96 (s, 9H) |
| I-78^{b} | BV | H | 1254.0 | 12.49-11.69 (m, 1H), 8.99 (s, 1H), 8.89 (d, J = 4.1 Hz, 1H), |
| | | | | 8.54-8.47 (m, 1H), 8.40 (d, J = 7.9 Hz, 1H), 8.12-8.01 (m, |
| | | | | 1H), 7.96 (d, J = 8.7 Hz, 1H), 7.88 (d, J = 9.2 Hz, 1H), 7.47- |
| | | | | 7.34 (m, 6H), 7.33-7.22 (m, 1H), 6.88-6.79 (m, 2H), 6.63- |
| | | | | 6.56 (m, 1H), 5.09-4.97 (m, 1H), 4.97-4.85 (m, 1H), 4.53 (d, |
| | | | | J = 9.1 Hz, 1H), 4.43 (t, J = 8.3 Hz, 1H), 4.39-4.35 (m, 1H), |
| | | | | 4.30-4.25 (m, 2H), 4.05-3.97 (m, 1H), 3.92-3.82 (m, 1H), |
| | | | | 3.73-3.67 (m, 2H), 3.66-3.57 (m, 2H), 3.48-3.25 (m, 3H), |
| | | | | 2.46 (s, 3H), 2.32-2.24 (m, 4H), 2.23-2.17 (m, 12H), 2.17- |
| | | | | 2.10 (m, 2H), 2.07-1.97 (m, 1H), 1.89-1.64 (m, 8H), 1.38 (d, |
| | | | | J = 7.0 Hz, 3H), 0.95 (s, 9H) |
| I-79 | BT | H | 1258.6 | 12.10-11.83 (m, 1H), 9.00 (s, 1H), 8.94-8.80 (m, 1H), 8.56- |
| | | | | 8.43 (m, 1H), 8.44-8.33 (m, 1H), 8.17-8.03 (m, 1H), 8.00- |
| | | | | 7.85 (m, 2H), 7.52-7.36 (m, 6H), 7.32-7.10 (m, 3H), 7.05- |
| | | | | 6.93 (m, 1H), 6.86-6.76 (m, 1H), 6.74-6.60 (m, 1H), 5.10- |
| | | | | 4.98 (m, 1H), 4.97-4.81 (m, 1H), 4.60-4.50 (m, 1H), 4.41- |
| | | | | 4.36 (m, 2H), 4.33-4.17 (m, 2H), 4.14-3.82 (m, 4H), 3.66- |
| | | | | 3.59 (m, 4H), 2.58-2.56 (m, 1H), 2.47-2.41 (m, 4H), 2.36- |
| | | | | 1.99 (m, 12H), 1.93-1.61 (m, 9H), 1.39-1.36 (m, 3H), 0.96 |
| | | | | (s, 9H) |
| I-80^{f} | BR | H | 1272.5 | 10.53 (d, J = 20.0 Hz, 1H), 9.41 (d, J = 10.0 Hz, 1H), 8.67 |
| | | | | (d, J = 13.7 Hz, 5H), 8.48 (d, J = 7.7 Hz, 1H), 7.81 (d, J = |
| | | | | 9.2 Hz, 1H), 7.52-7.39 (m, 6H), 7.23 (s, 1H), 7.18-6.91 (m, |
| | | | | 3H), 6.77-6.65 (m, 1H), 5.23 (dd, J = 11.1, 2.9 Hz, 1H), 4.94 |
| | | | | (t, J = 7.2 Hz, 1H), 4.59-4.40 (m, 3H), 4.43-4.24 (m, 2H), |
| | | | | 4.18 (d, J = 9.4 Hz, 1H), 3.65 (d, J = 17.5 Hz, 2H), 3.52-3.26 |
| | | | | (m, 2H), 3.14 (s, 2H), 3.00 (d, J = 16.9 Hz, 1H), 2.40-1.70 |
| | | | | (m, 8H), 1.67-1.43 (m, 8H), 1.39 (d, J = 6.9 Hz, 3H), 1.33- |
| | | | | 1.18 (m, 2H), 1.18-1.00 (m, 1H), 0.94 (s, 9H) |
| I-90^{d} | DQ | A | 1046.7 | 11.10-11.06 (m, 1H), 8.99-8.96 (m, 1H), 8.90-8.86 (m, 1H), |
| | | | | 8.41 (s, 1H), 8.23-8.19 (m, 1H), 8.17-8.13 (m, 1H), 8.03- |
| | | | | 7.99 (m, 1H), 7.23-7.16 (m, 2H), 7.04-7.00 (m, 1H), 7.00- |
| | | | | 6.96 (m, 2H), 6.90-6.86 (m, 2H), 6.75 (s, 1H), 5.36-5.32 (m, |
| | | | | 1H), 4.97-4.93 (m, 1H), 4.33-4.29 (m, 1H), 4.15-4.06 (m, |
| | | | | 1H), 4.03-3.99 (m, 1H), 3.93-3.89 (m, 1H), 3.32 (s, 3H), |
| | | | | 2.92-2.88 (m, 1H), 2.73-2.56 (m, 6H), 2.22-2.18 (m, 2H), |
| | | | | 2.06-1.96 (m, 3H), 1.92-1.81 (m, 8H), 1.63-1.59 (m, 11H), |
| | | | | 1.42-1.31 (m, 2H) |
| I-92^{c,d} | DQ | AW | 1105.9 | 11.08 (s, 1H), 9.15-9.02 (m, 1H), 8.91 (s, 1H), 8.42-8.36 (m, |
| | | | | 1H), 8.22-8.14 (m, 2H), 8.04-7.99 (m, 1H), 7.21-7.12 (m, |
| | | | | 2H), 7.03-6.95 (m, 3H), 6.94-6.81 (m, 2H), 6.78-6.71 (m, |
| | | | | 1H), 5.37-5.30 (m, 1H), 4.96-4.87 (m, 1H), 4.37-4.23 (m, |
| | | | | 2H), 4.10-3.98 (m, 2H), 3.95-3.87 (m, 1H), 3.83-3.74 (m, |
| | | | | 2H), 3.68-3.63 (m, 3H), 3.58-3.44 (m, 4H), 3.31 (s, 3H), |
| | | | | 2.95-2.85 (m, 1H), 2.69-2.58 (m, 4H), 2.21-1.97 (m, 6H), |
| | | | | 1.92-1.79 (m, 3H), 1.76-1.54 (m, 7H), 1.40-1.31 (m, 2H), |
| | | | | 1.24-1.14 (m, 1H) |
| I-94^{e} | DQ | H | 1110.8 | 11.07 (s, 1H), 9.09-8.70 (m, 1H), 8.345-8.24 (m, 1H), 8.14- |
| | | | | 7.97 (m, 3H), 7.69-7.53 (m, 1H), 7.27-7.14 (m, 2H), 7.07- |
| | | | | 6.93 (m, 3H), 6.92-3.81 (m, 2H), 6.80-6.62 (m, 1H), 5.37- |
| | | | | 5.28 (m, 1H), 4.02-4.89 (m, 1H), 4.40-4.28 (m, 1H), 4.27- |
| | | | | 4.17 (m, 1H), 4.11-3.95 (m, 2H), 3.95-3.80 (m, 2H), 3.76- |
| | | | | 3.60 (m, 3H), 3.44-3.38 (m, 1H), 3.39 (s, 3H), 2.96-2.81 (m, |
| | | | | 1H), 2.80-2.54 (m, 6H), 2.24-2.06 (m, 6H), 2.05-1.96 (m, |
| | | | | 2H), 1.93-1.81 (m, 3H), 1.76-1.68 (m, 2H), 1.67-1.52 (m, |
| | | | | 5H), 1.41-1.31 (m, 2H), 1.25-1.14 (m, 1H) |

^{a}Intermediate B was coupled in Step 3 for all examples shown. Step 1 was run anywhere from 2-16 h at rt. Step 2 was run anywhere from 2-4 h at rt; dioxane or THF used as the solvent. Step 3 was run anywhere from 2-16 h, DMF or NMP used as the solvent.
^{b}[(M − H)]⁻ LCMS ion reported not [(M + H)]⁺.
^{c}In Step 2, TFA in DCM at rt for 2 hr was used for the deprotection.
^{d}TEA in NMP was used for the Step 3 coupling.
^{e}Intermediate BA was used instead of Intermediate B in Step 3.
^{f}[(M + 2)]⁺ LCMS ion reported not [(M + H)]⁺.

### Example 3 (Method 2). Synthesis of 2-[[(2S)-1-[(2S)-2-[[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamoyl]pyrrolidin-1-yl]-3-methyl-1-oxobutan-2-yl]carbamoyl]-1H-indole-5-carbonylphosphonic acid (I-15)

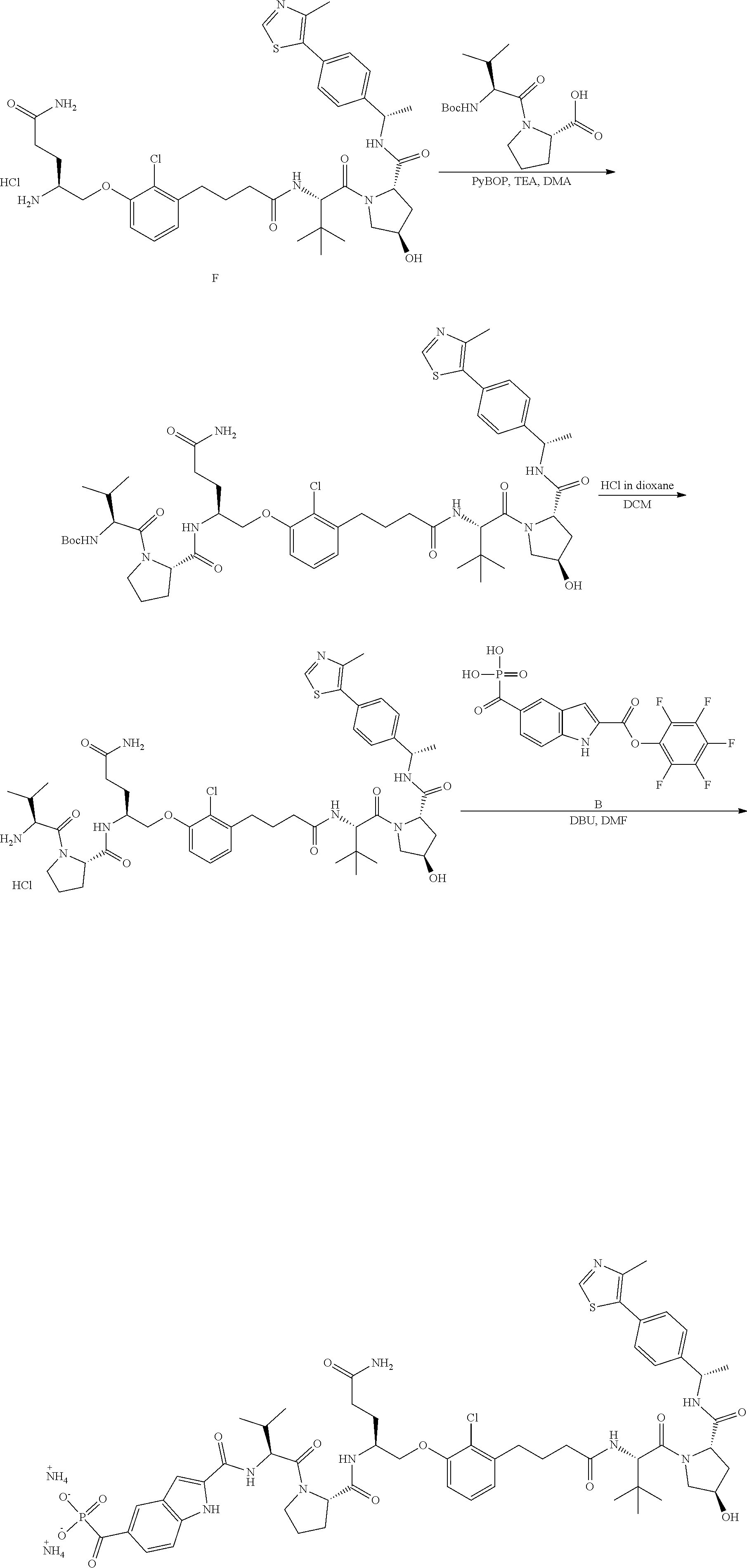

Step 1—Tert-butyl N-[(2S)-1-[(2S)-2-[[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamoyl]pyrrolidin-1-yl]-3-methyl-1-oxobutan-2-yl]carbamate. To a stirred solution of (2S,4R)-1-[(2S)-2-(4-[3-[(2S)-2-amino-4-carbamoylbutoxy]-2-chlorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (250.00 mg, 0.32 mmol, Intermediate F), TEA (95.84 mg, 0.947 mmol) and (2S)-1-[(2S)-2-[(tert-butoxycarbonyl)amino]-3-methylbutanoyl]pyrrolidine-2-carboxylic acid (119.11 mg, 0.38 mmol, CAS #23361-28-6) in DMA (4.00 mL) was added PyBOP (246.45 mg, 0.47 mmol) at rt under nitrogen atmosphere. The resulting mixture was stirred for 4 h at rt under nitrogen atmosphere. On completion, the residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 35%-55% B in 15 min; Flow rate: 55 mL/min; Detector: 220/254 nm; desired fractions were collected at 45% B) and concentrated under reduced pressure to afford the title compound. ¹H NMR (300 MHz, DMSO-d₆) δ 8.98 (s, 1H), 8.36 (d, J=7.9 Hz, 1H), 7.90-7.81 (m, 2H), 7.47-7.33 (m, 4H), 7.24-7.08 (m, 2H), 7.01-6.84 (m, 2H), 6.81-6.68 (m, 2H), 4.96-4.85 (m, 1H), 4.56-4.36 (m, 2H), 4.31-4.25 (m, 2H), 4.12-3.83 (m, 5H), 3.73-3.64 (m, 1H), 3.63-3.58 (m, 2H), 2.62-2.60 (m, 2H), 2.45 (s, 3H), 2.37-2.11 (m, 5H), 2.01-1.61 (m, 11H), 1.40-1.31 (m, 12H), 0.99-0.76 (m, 15H). LC/MS (ESI, m z): [(M−1)]⁻=1049.6.

Step 2—(2S,4R)-1-[(2S)-2-(4-[3-[(2S)-2-[[(2S)-1-[(2S)-2-amino-3-methylbutanoyl]pyrrolidin-2-yl]formamido]-4-carbamoylbutoxy]-2-chlorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride. To a stirred solution of tert-butyl N-[(2S)-1-[(2S)-2-[[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamoyl]pyrrolidin-1-yl]-3-methyl-1-oxobutan-2-yl]carbamate (150.00 mg) in DCM (4.00 mL) was added 4 M HCl (gas) in 1,4-dioxane (2.00 mL) at rt and the mixture was stirred for 1 h at rt. On completion, the resulting mixture was concentrated under reduced pressure to give the title compound (100 mg, crude) as a white solid. ¹H NMR (300 MHz, DMSO-d₆) δ 9.08-9.01 (m, 1H), 8.38 (d, J=7.8 Hz, 1H), 8.19-8.13 (m, 2H), 8.08-7.99 (m, 1H), 7.86 (d, J=9.2 Hz, 1H), 7.48-7.34 (m, 4H), 7.24-7.11 (m, 2H), 7.01-6.84 (m, 2H), 6.80-6.67 (m, 1H), 4.94-4.88 (m, 1H), 4.54-4.51 (m, 1H), 4.44-4.35 (m, 2H), 4.31-4.18 (m, 2H), 4.13-3.85 (m, 5H), 3.76-3.66 (m, 1H), 3.65-3.58 (m, 2H), 2.72-2.61 (m, 2H), 2.45 (s, 3H), 2.35-2.23 (m, 1H), 2.22-1.89 (m, 8H), 1.87-1.62 (m, 7H), 1.37 (d, J=7.1 Hz, 3H), 1.05-0.87 (m, 15H). LC/MS (ESI, m/z): [(M+1)]⁺=951.5.

Step 3—Diammonium 2-[[(2S)-1-[(2S)-2-[[(2S)-4-carbamoyl-1-[2-chloro-3-(3-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]propyl)phenoxy]butan-2-yl]carbamoyl]pyrrolidin-1-yl]-3-methyl-1-oxobutan-2-yl]carbamoyl]-1H-indole-5-carbonylphosphonate. To a stirred mixture of (2S,4R)-1-[(2S)-2-(4-[3-[(2S)-2-[[(2S)-1-[(2S)-2-amino-3-methylbutanoyl]pyrrolidin-2-yl]formamido]-4-carbamoylbutoxy]-2-chlorophenyl]butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (100.00 mg, 0.10 mmol) and 2-(2,3,4,5,6-pentafluorophenoxycarbonyl)-1H-indole-5-carbonylphosphonic acid (132.13 mg, 0.30 mmol, Intermediate B) in NMP (6.00 mL) was added DBU (77.04 mg, 0.51 mmol) dropwise at rt under nitrogen atmosphere. The resulting mixture was stirred for 3h at rt under nitrogen atmosphere. On completion, the residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 20%-50% B in 30 min; Flow rate: 60 mL/min; Detector: 220/254 nm; desired fractions were collected at 32% B) and concentrated under reduced pressure to afford the title compound (36.2 mg, 30% yield) as a white solid. ¹H NMR (400 MHz, DMSO-d₆) δ 11.97 (s, 1H), 8.99 (s, 1H), 8.86 (s, 1H), 8.77-8.73 (m, 1H), 8.41 (d, J=7.9 Hz, 1H), 7.97-7.88 (m, 2H), 7.48-7.36 (m, 7H), 7.24-7.17 (m, 2H), 7.03-6.77 (m, 3H), 4.96-4.88 (m, 1H), 4.58-4.49 (m, 2H), 4.48-4.44 (m, 1H), 4.35-4.26 (m, 2H), 4.11-4.06 (m, 2H), 4.04-3.86 (m, 5H), 3.71-3.66 (m, 1H), 3.63-3.60 (m, 2H), 2.72-2.63 (m, 2H), 2.46 (s, 3H), 2.36-2.26 (m, 1H), 2.23-2.17 (m, 3H), 2.25-2.13 (m, 4H), 2.09-1.65 (m, 11H), 1.38 (d, J=6.9 Hz, 3H), 1.07-0.88 (m, 15H). LC/MS (ESI, m z): [(M−1)]−=1200.6.

TABLE 4
Compounds synthesized via Method 2, where the corresponding amines and acids were
coupled in Step 1.

| | | | LC/MS | |
|---|---|---|---|---|
| | Step 1 | | (ESI, m/z) | |
| I-# | Amine | Step 1 Acid | (M + H)⁺ | 1H NMR (400 MHZ, DMSO-d6) δ |
| I-5 | X | A | 1227.1 | 11.82 (s, 1H), 8.99 (s, 1H), 8.88 (s, 1H), 8.66 (d, J = 7.1 |
| | | | | Hz, 1H), 8.39 (d, J = 7.8 Hz, 1H), 8.01 (d, J = 8.3 Hz, |
| | | | | 1H), 7.94 (dd, J = 8.7, 1.6 Hz, 1H), 7.87 (d, J = 9.2 Hz, |
| | | | | 1H), 7.46-7.09 (m, 11H), 6.87-6.66 (m, 2H), 5.06-4.97 |
| | | | | (m, 1H), 4.95-4.89 (m, 1H), 4.54 (d, J = 9.3 Hz, 1H), 4.43 |
| | | | | (t, J = 8.0 Hz, 1H), 4.38-4.25 (m, 3H), 4.12-4.06 (m, 1H), |
| | | | | 4.01-3.97 (m, 1H), 3.91-3.87 (m, 1H), 3.64-3.59 (m, 2H), |
| | | | | 2.70-2.58 (m, 1H), 2.46 (s, 3H), 2.37-2.12 (m, 8H), 2.11- |
| | | | | 1.96 (m, 3H), 1.96-1.64 (m, 12H), 1.61-1.48 (m, 3H), |
| | | | | 1.38 (d, J = 7.0 Hz, 3H), 0.95 (s, 9H) |
| I-6 | Q | A | 1224.2 | 11.81 (s, 1H), 8.99 (s, 1H), 8.89 (s, 1H), 8.66 (d, J = 7.1 |
| | | | | Hz, 1H), 8.40 (d, J = 7.8 Hz, 1H), 8.04 (d, J = 8.3 Hz, |
| | | | | 1H), 7.94 (d, J = 8.7 Hz, 1H), 7.81 (d, J = 9.3 Hz, 1H), |
| | | | | 7.48-7.33 (m, 7H), 7.19 (s, 1H), 7.06-6.94 (m, 2H), 6.84- |
| | | | | 6.80 (m, 1H), 6.77 (s, 1H), 5.05-4.99 (m, 1H), 4.96-4.88 |
| | | | | (m, 1H), 4.51 (d, J = 9.4 Hz, 1H), 4.43 (t, J = 8.0 Hz, |
| | | | | 1H), 4.37-4.24 (m, 3H), 4.09-4.04 (m, 1H), 4.01-3.97 (m, |
| | | | | 1H), 3.93-3.88 (m, 1H), 3.63-3.58 (m, 2H), 2.61-2.55 (m, |
| | | | | 2H), 2.46 (s, 3H), 2.31-1.99 (m, 8H), 1.95-1.75 (m, 8H), |
| | | | | 1.73-1.66 (m, 3H), 1.60-1.46 (m, 7H), 1.38 (d, J = 7.0 |
| | | | | Hz, 3H), 1.30-1.25 (m, 2H), 0.93 (s, 9H) |
| I-11 | F | G | 1188.6^{b} | 11.95-11.77 (m, 1H), 9.03-8.97 (m, 1H), 8.92-8.79 (m, |
| | | | | 2H), 8.54-8.37 (m, 1H), 8.02-7.77 (m, 3H), 7.49-7.35 (m, |
| | | | | 6H), 7.28-7.18 (m, 2H), 7.04-6.97 (m, 1H), 6.94-6.88 (m, |
| | | | | 1H), 6.83-6.76 (m, 1H), 5.00-4.84 (m, 2H), 4.59-4.35 (m, |
| | | | | 3H), 4.32-4.26 (m, 1H), 4.10-3.90 (m, 3H), 3.85-3.76 (m, |
| | | | | 3H), 3.66-3.60 (m, 2H), 3.56-3.51 (m, 1H), 3.48-3.43 (m, |
| | | | | 1H), 3.27-3.20 (m, 1H), 2.72-2.65 (m, 2H), 2.47 (s, 3H), |
| | | | | 2.39-2.12 (m, 5H), 2.10-2.00 (m, 2H), 1.96-1.87 (m, 3H), |
| | | | | 1.83-1.72 (m, 3H), 1.69-1.54 (m, 3H), 1.38 (d, J = 7.0 |
| | | | | Hz, 3H), 0.96 (s, 9H) |
| I-12 | F | DD | 1240.6 | 12.29-11.46 (m, 1H), 9.00 (s, 1H), 8.89 (s, 1H), 8.77-8.68 |
| | | | | (m, 1H), 8.42 (d, J = 7.8 Hz, 1H), 8.15-7.86 (m, 3H), |
| | | | | 7.50-7.33 (m, 6H), 7.29-7.17 (m, 2H), 7.00 (d, J = 8.2 |
| | | | | Hz, 1H), 6.91 (d, J = 7.7 Hz, 1H), 6.80 (s, 1H), 4.99-4.88 |
| | | | | (m, 1H), 4.63-4.51 (m, 3H), 4.51-4.40 (m, 2H), 4.37-4.26 |
| | | | | (m, 3H), 4.19-3.90 (m, 7H), 3.80-3.47 (m, 6H), 2.78-2.61 |
| | | | | (m, 3H), 2.48 (s, 3H), 2.36-2.19 (m, 4H), 2.11-1.96 (m, |
| | | | | 2H), 1.93-1.57 (m, 8H), 1.39 (d, J = 6.9 Hz, 3H), 1.27- |
| | | | | 1.02 (m, 4H), 0.97 (s, 9H) |
| I-13 | F | CA | [M − H)⁻ = | 11.88 (s, 1H), 9.01-8.95 (m, 2H), 8.83-8.67 (m, 1H), |
| | | | 1159.4 | 8.50-8.30 (m, 1H), 7.96-7.90 (m, 2H), 7.47-7.16 (m, 9H), |
| | | | | 7.01-7.98 (m, 1H), 6.89-6.77 (m, 2H), 5.05-4.83 (m, |
| | | | | 1H), 4.61-4.39 (m, 3H), 4.38-4.15 (m, 4H), 4.15-3.90 (m, |
| | | | | 5H), 3.81-3.56 (m, 5H), 2.80-2.60 (m, 2H), 2.47-2.42 (m, |
| | | | | 3H), 2.41-2.12 (m, 4H), 2.12-1.70 (m, 9H), 1.39-1.35 (m, |
| | | | | 3H), 0.96 (s, 9H) |
| I-14 | F | (2S)-1-[(2S)- | 1214.4^{b} | 12.05 (s, 1H), 9.01-8.96 (m, 1H), 8.88 (s, 1H), 8.44-8.37 |
| | | 2-[(tert- | | (m, 1H), 8.23-8.16 (m, 1H), 7.99-7.95 (m, 1H), 7.92-7.87 |
| | | butoxycarbonyl) | | (m, 1H), 7.54-7.33 (m, 7H), 7.25-7.17 (m, 2H), 7.01-6.96 |
| | | amino]-3, 3- | | (m, 1H), 6.93-6.86 (m, 1H), 6.81-6.76 (m, 1H), 4.96-4.81 |
| | | dimethylbutanoyl] | | (m, 2H), 4.57-4.51 (m, 1H), 4.48-4.39 (m, 1H), 4.36-4.25 |
| | | pyrrolidine-2- | | (m, 2H), 4.14-4.08 (m, 1H), 4.04-3.98 (m, 1H), 4.96- |
| | | carboxylic acid | | 3.89 (m, 2H), 3.85-3.80 (m, 1H), 3.72-3.67 (m, 1H), |
| | | (CAS# 2389052- | | 3.64-3.59 (m, 2H), 2.71-2.64 (m, 2H), 2.46 (s, 3H), 2.36- |
| | | 05-3) | | 2.13 (m, 5H), 2.06-1.65 (m, 11H), 1.37 (d, J = 6.9 Hz, |
| | | | | 3H), 1.07 (s, 9H), 0.94 (s, 9H) |
| I-53 | F | AB | 1227.2^{b} | 11.96 (s, 1H), 8.99 (s, 1H), 8.89 (s, 1H), 8.78-8.72 (m, |
| | | | | 1H), 8.42 (d, J = 7.8 Hz, 1H), 7.99-7.87 (m, 2H), 7.47- |
| | | | | 7.36 (m, 7H), 7.31-7.19 (m, 2H), 7.01 (d, J = 8.3 Hz, 1H), |
| | | | | 6.91 (d, J = 7.6 Hz, 1H), 6.82 (s, 1H), 5.22-5.16 (m, 1H), |
| | | | | 4.99-4.86 (m, 1H), 4.60-4.50 (m, 1H), 4.49-4.39 (m, 2H), |
| | | | | 4.35-4.27 (m, 2H), 4.14-4.08 (m, 1H), 4.00-3.84 (m, 3H), |
| | | | | 3.66-3.57 (m, 3H), 3.02-2.88 (m, 2H), 2.86-2.60 (m, |
| | | | | 5H), 2.46 (s, 3H), 2.45-2.42 (m, 2H), 2.37-2.14 (m, 6H), |
| | | | | 2.07-1.98 (m, 2H), 1.96-1.66 (m, 8H), 1.63-1.54 (m, |
| | | | | 1H), 1.38 (s, 3H), 0.95 (s, 9H) |
| I-54 | F | DK | (M − H) | 12.06 (s, 1H), 8.99 (s, 1H), 8.92-8.88 (m, 1H), 8.45-8.41 |
| | | | 1283.3 | (m, 2H), 8.12-8.08 (m, 1H), 7.99-7.95 (m, 1H), 7.91-7.87 |
| | | | | (m, 1H), 7.49-7.32 (m, 4H), 7.30-7.13 (m, 2H), 7.06-6.97 |
| | | | | (m, 1H), 6.92-6.88 (m, 1H), 6.80 (s, 1H), 4.96-4.92 (m, |
| | | | | 2H), 4.57-4.53 (m, 1H), 4.45-4.41 (m, 2H), 4.31-4.27 (m, |
| | | | | 1H), 4.21-4.17 (m, 1H), 4.09-3.98 (m, 2H), 3.97-3.93 (m, |
| | | | | 1H), 3.66-3.20 (m, 6H), 2.71-2.67 (m, 3H), 2.47 (s, 3H), |
| | | | | 2.35-2.12 (m, 7H), 1.92-1.57 (m, 14H), 1.41-1.37 (m, |
| | | | | 3H), 1.17-1.00 (m, 6H), 0.96 (s, 9H) |
| I-55 | V | R | 1288.6 | 11.88 (s, 1H), 9.00-8.92 (m, 3H), 8.39 (d, J = 7.8 Hz, |
| | | | | 1H), 8.13 (d, J = 7.4 Hz, 1H), 7.95 (d, J = 8.8 Hz, 1H), |
| | | | | 7.85 (d, J = 9.4 Hz, 1H), 7.50-7.34 (m, 6H), 7.25-6.95 |
| | | | | (m, 10H), 6.76 (s, 1H), 5.10 (d, J = 10.6 Hz, 1H), 4.93 (t, |
| | | | | J = 7.3 Hz, 1H), 4.72-4.65 (m, 1H), 4.55 (d, J = 8.7 Hz, |
| | | | | 1H), 4.45 (t, J = 7.9 Hz, 1H), 4.29 (s, 1H), 4.13-3.89 (m, |
| | | | | 3H), 3.63 (s, 2H), 3.51-3.46 (m, 1H), 3.22-3.09 (m, 3H), |
| | | | | 3.04-2.84 (m, 2H), 2.47 (s, 3H), 2.33-1.96 (m, 7H), 1.96- |
| | | | | 1.66 (m, 8H), 1.51-1.43 (m, 1H), 1.39 (d, J = 6.7 Hz, 3H), |
| | | | | 1.16-1.08 (m, 2H), 0.97 (s, 9H) |
| I-56 | CY | R | 1290.5 | 11.91 (s, 1H), 9.03-8.98 (m, 1H), 8.97-8.88 (m, 2H), |
| | | | | 8.51-8.43 (m, 1H), 8.18-8.11 (m, 1H), 7.99-7.92 (m, 1H), |
| | | | | 7.47-7.36 (m, 6H), 7.30-7.21 (m, 3H), 7.13-6.95 (m, 5H), |
| | | | | 6.79-6.73 (m, 1H), 5.14-5.05 (m, 1H), 4.95-4.86 (m, 1H), |
| | | | | 4.73-4.64 (m, 1H), 4.57-4.41 (m, 2H), 4.33-4.27 (m, 1H), |
| | | | | 4.07-3.95 (m, 3H), 3.63-3.58 (m, 2H), 3.48-3.27 (m, 6H), |
| | | | | 3.23-3.13 (m, 3H), 3.07-2.91 (m, 5H), 2.46 (m, 3H), |
| | | | | 2.30-2.20 (m, 2H), 2.18-2.02 (m, 3H), 1.92-1.65 (m, 7H), |
| | | | | 1.39 (d, J = 7.0 Hz, 3H), 0.97 (s, 9H) |
| I-58 | F | DC | 1174.4 | 12.04 (s, 1H), 8.99 (s, 1H), 8.83-8.79 (m, 2H), 8.40-8.36 |
| | | | | (m, 1H), 7.94-7.90 (m, 2H), 7.46-7.42 (m, 4H), 7.31-7.27 |
| | | | | (m, 2H), 7.22-7.18 (m, 2H), 7.14-7.10 (m, 2H), 7.02-6.86 |
| | | | | (m, 4H), 6.80-6.76 (m, 1H), 5.35-5.31 (m, 1H), 4.98-4.80 |
| | | | | (m, 1H), 4.56-4.43 (m, 2H), 4.37-4.26 (m, 2H), 4.06-3.90 |
| | | | | (m, 2H), 3.66-3.58 (m, 3H), 2.70-2.66 (m, 2H), 2.48-2.44 |
| | | | | (m, 3H), 2.19-2.15 (m, 2H), 2.05-1.95 (m, 4H), 1.80-1.70 |
| | | | | (m, 6H), 1.30-1.22 (m, 10H), 0.95 (s, 9H) |
| I-60 | F | T | 1337.5 | 11.79 (s, 1H), 9.00 (s, 1H), 8.88 (s, 1H), 8.67 (d, J = 7.1 |
| | | | | Hz, 1H), 8.40 (d, J = 7.8 Hz, 1H), 8.09 (d, J = 7.7 Hz, |
| | | | | 1H), 8.02-7.85 (m, 2H), 7.48-7.38 (m, 5H), 7.33-7.26 (m, |
| | | | | 3H), 7.24-7.11 (m, 4H), 7.06-7.02 (m, 1H), 6.92-6.88 (m, |
| | | | | 1H), 6.75 (s, 1H), 5.32-5.28 (m, 1H), 5.18-5.13 (m, 1H), |
| | | | | 4.96-4.91 (m, 1H), 4.57-4.53 (m, 2H), 4.47-4.40 (m, 2H), |
| | | | | 4.37-4.30 (m, 2H), 4.15-4.39 (m, 5H), 3.62-3.48 (m, 4H), |
| | | | | 2.71-2.68 (m, 2H), 2.48 (s, 3H), 2.40-1.96 (m, 8H), 1.90- |
| | | | | 1.73 (m, 9H), 1.39 (d, J = 6.9 Hz, 3H), 0.96 (s, 9H) |
| I-61^{d} | F | S | 1238.5^{b} | 12.02-11.82 (m, 1H), 9.03-8.97 (m, 2H), 8.91-8.86 (m, |
| | | | | 1H), 8.55-8.38 (m, 2H), 8.11-8.06 (m, 1H), 7.98-7.86 (m, |
| | | | | 2H), 7.47-7.16 (m, 10H), 7.01-6.88 (m, 2H), 6.85-6.80 |
| | | | | (m, 1H), 4.99-4.88 (m, 2H), 4.58-4.41 (m, 2H), 4.37-4.27 |
| | | | | (m, 2H), 4.14-4.09 (m, 1H), 4.02-3.90 (m, 2H), 3.75-3.60 |
| | | | | (m, 3H), 3.47-3.44 (m, 1H), 3.18-2.99 (m, 3H), 2.72-2.66 |
| | | | | (m, 2H), 2.47 (s, 3H), 2.39-2.27 (m, 1H), 2.25-2.15 (m, |
| | | | | 3H), 2.12-1.97 (m, 3H), 1.96-1.66 (m, 9H), 1.39 (d, J = |
| | | | | 6.9 Hz, 3H), 0.96 (s, 9H) |
| I-62^{c} | AP | 1-[4- | 1184.4 | 12.15-11.08 (m, 1H), 11.09 (s, 1H), 8.84 (s, 1H), 8.59- |
| | | (ethanesulfonyl) | | 8.50 (m, 2H), 8.34-8.27 (m, 1H), 7.99 (d, J = 8.9 Hz, 1H), |
| | | phenyl] | | 7.89-7.78 (m, 2H), 7.57-7.52 (m, 4H), 7.46 (d, J = 1.6 |
| | | methanamine | | Hz, 1H), 7.30-7.27 (m, 1H), 7.06-6.93 (m, 2H), 6.90-6.70 |
| | | (CAS# | | (m, 2H), 5.34 (dd, J = 12.7, 5.1 Hz, 1H), 5.01-4.88 (m, |
| | | 583837-94-9) | | 1H), 4.50-4.41 (m, 3H), 4.31-4.18 (m, 2H), 4.02-3.74 (m, |
| | | | | 1H), 3.38-3.22 (m, 9H), 2.94-2.87 (m, 1H), 2.79-2.55 (m, |
| | | | | 5H), 2.22-2.10 (m, 3H), 2.08-1.96 (m, 3H), 1.94-1.57 (m, |
| | | | | 7H), 1.48-1.25 (m, 9H), 1.10 (t, J = 7.3 Hz, 3H) |
| I-64 | O | N | 1181.5 | 12.16-12.01 (m, 1H), 8.83-8.79 (m, 2H), 8.51-8.47 (m, |
| | | | | 1H), 8.30-8.21 (m, 1H), 7.98 (d, J = 9.0 Hz, 1H), 7.57- |
| | | | | 7.53 (m, 1H), 7.46-7.42 (m, 1H), 7.37-7.26 (m, 11H), |
| | | | | 6.99-6.74 (m, 2H), 6.89-.71 (m, 2H), 6.12 (d, J = 8.3 Hz, |
| | | | | 1H), 5.44-5.40 (m, 1H), 5.03-4.98 (m, 1H), 4.50-4.35 (m, |
| | | | | 2H), 4.23-4.19 (m, 1H), 4.10-3.88 (m, 1H), 3.83-3.79 (m, |
| | | | | 1H), 3.32-3.28 (m, 4H), 3.05 (s, 3H), 2.98-2.95 (m, 1H), |
| | | | | 2.83-2.79 (m, 1H), 2.80-2.69 (m, 1H), 2.64-2.52 (m, |
| | | | | 3H), 2.16-1.99 (m, 5H), 1.97-1.92 (m, 7H), 1.80-1.64 (m, |
| | | | | 4H), 1.49-1.29 (m, 9H) |
| I-66 | F | CS | 1311.4 | 11.85 (s, 1H), 8.98 (s, 1H), 8.93-8.82 (m, 2H), 8.43-8.36 |
| | | | | (m, 1H), 8.07-7.94 (m, 2H), 7.93-7.85 (m, 1H), 7.46-7.35 |
| | | | | (m, 6H), 7.24-7.15 (m, 2H), 7.03-6.98 (m, 1H), 6.93-6.87 |
| | | | | (m, 1H), 6.76-6.71 (m, 1H), 5.30-5.18 (m, 1H), 4.96-4.87 |
| | | | | (m, 1H), 4.58-4.50 (m, 1H), 4.48-4.37 (m, 2H), 4.36-4.25 |
| | | | | (m, 2H), 4.10-3.74 (m, 8H), 3.72-3.60 (m, 4H), 2.70-2.65 |
| | | | | (m, 2H), 2.45 (s, 3H), 2.34-2.11 (m, 6H), 2.11-1.96 (m, |
| | | | | 4H), 1.82-1.70 (m, 6H), 1.39-1.35 (m, 3H), 0.94 (s, 9H) |
| I-67 | L | M | 1142.5 | 12.08-11.76 (m, 1H), 9.01-8.79 (m, 1H), 8.54 (d, J = 6.6 |
| | | | | Hz, 1H), 8.22-8.07 (m, 1H), 7.97 (d, J = 8.6 Hz, 1H), |
| | | | | 7.90-7.79 (m, 2H), 7.48-7.36 (m, 2H), 7.34-7.07 (m, 4H), |
| | | | | 7.05-6.93 (m, 2H), 6.89-6.70 (m, 2H), 5.41-5.31 (m, 1H), |
| | | | | 5.09-4.89 (m, 1H), 4.46-4.21 (m, 2H), 4.13-3.85 (m, 5H), |
| | | | | 3.80-3.77 (m, 2H), 3.44 (s, 3H), 3.17 (s, 3H), 3.00-2.85 |
| | | | | (m, 2H), 2.73-2.57 (m, 3H), 2.31-1.97 (m, 8H), 1.92-1.61 |
| | | | | (m, 9H), 1.52-1.30 (m, 8H) |
| I-71 | CR | R | 1244.7 | 11.96-11.84 (m, 1H), 8.99-8.94 (m, 3H), 8.84 (s, 1H), |
| | | | | 8.16 (d, J = 7.4 Hz, 1H), 8.05 (d, J = 9.3 Hz, 1H), 7.99- |
| | | | | 7.83 (m, 1H), 7.49-7.42 (m, 3H), 7.36-7.28 (m, 4H), |
| | | | | 7.25-7.14 (m, 1H), 7.12-7.05 (m, 2H), 7.06-7.01 (m, 2H), |
| | | | | 6.96-6.87 (m, 1H), 6.83-6.76 (m, 1H), 5.14-5.07 (m, 1H), |
| | | | | 4.73-4.67 (m, 1H), 4.63-4.56 (m, 1H), 4.46-4.38 (m, 2H), |
| | | | | 4.08-3.95 (m, 3H), 3.74-3.68 (m, 2H), 3.23-3.17 (m, 3H), |
| | | | | 3.01-2.96 (m, 1H), 2.71-2.67 (m, 2H), 2.45 (s, 3H), 2.36- |
| | | | | 1.99 (m, 9H), 1.92-1.66 (m, 6H), 1.24-1.16 (m, 4H), 0.97 |
| | | | | (s, 9H) |
| I-72 | CR | H | 1269.5 | 11.96-11.88 (m, 1H), 9.02-8.97 (m, 1H), 8.95-8.90 (m, |
| | | | | 1H), 8.86-8.79 (m, 1H), 8.51 (d, J = 6.8 Hz, 1H), 8.11- |
| | | | | 7.95 (m, 3H), 7.43 (d, J = 8.4 Hz, 1H), 7.37-7.29 (m, 5H), |
| | | | | 7.26-7.18 (m, 1H), 7.14-6.62 (m, 4H), 5.07-5.01 (m, 1H), |
| | | | | 4.58 (d, J = 9.1 Hz, 1H), 4.45-4.38 (m, 4H), 4.05 (d, J = |
| | | | | 9.9 Hz, 2H), 3.98-3.83 (m, 3H), 3.71-3.67 (m, 5H), 2.68 |
| | | | | (d, J = 8.1 Hz, 2H), 2.45 (s, 3H), 2.36-2.11 (m, 9H), 2.04- |
| | | | | 1.61 (m, 12H), 1.26-1.16 (m, 4H), 0.96 (s, 9H) |
| I-74 | CN | CP | 1170.4 | 12.18-12.01 (m, 1H), 11.10 (s, 1H), 8.81 (s, 1H), 8.58- |
| | | | | 8.54 (m, 1H), 8.45 (d, J = 6.8 Hz, 1H), 8.30-8.26 (m, 1H), |
| | | | | 7.99-7.94 (m, 1H), 7.89-7.85 (m, 2H), 7.60-7.40 (m, 4H), |
| | | | | 7.32-7.24 (m, 1H), 7.06-6.91 (m, 2H), 6.87-6.68 (m, 2H), |
| | | | | 5.35 (dd, J = 12.8, 5.4 Hz, 1H), 4.99-4.84 (m, 1H), 4.51- |
| | | | | 4.35 (m, 4H), 4.31-4.11 (m, 4H), 3.96-3.86 (m, 2H), |
| | | | | 3.82-3.78 (m, 1H), 3.33 (s, 3H), 3.20 (s, 3H), 2.99-2.84 |
| | | | | (m, 2H), 2.77-2.63 (m, 3H), 2.31-1.91 (m, 8H), 1.90-1.35 |
| | | | | (m, 11H), 1.09-0.87 (m, 4H) |
| I-76 | CN | CM | 1143.4 | 12.17-11.98 (m, 1H), 11.09 (s, 1H), 8.87-8.79 (m, 1H), |
| | | | | 8.58-8.54 (m, 1H), 8.48-8.40 (m, 1H), 8.33-8.25 (m, 1H), |
| | | | | 7.98 (d, J = 8.8 Hz, 1H), 7.88-7.84 (m, 2H), 7.53-7.49 |
| | | | | (m, 3H), 7.44-7.41 (m, 1H), 7.32-7.25 (m, 1H), 7.06-6.97 |
| | | | | (m, 2H), 6.89-6.81 (m, 2H), 5.37-5.30 (m, 1H), 5.01-4.94 |
| | | | | (m, 1H), 4.48-4.37 (m, 3H), 4.29-4.20 (m, 2H), 3.96-3.73 |
| | | | | (m, 6H), 3.31 (s, 3H), 3.19 (s, 3H), 2.95-2.85 (m, 1H), |
| | | | | 2.77-2.57 (m, 6H), 2.22-2.09 (m, 3H), 2.03-1.77 (m, 6H), |
| | | | | 1.71-1.51 (m, 6H), 1.43-1.28 (m, 4H) |

^{a}Intermediate B was coupled in Step 3 for all examples shown unless otherwise noted. Step 1 was run anywhere from 1-4 h at rt. Step 2 was run anywhere from 1-4 h at rt; dioxane, EtOAc, THF or DCM used as the solvent; TFA could also be used for the deprotection in place of HCl. Step 3 was run anywhere from 1-16 h, DMF or NMP used as the solvent.
^{b}[(M − H)]⁻ LCMS ion reported not [(M + H)]⁺.
^{c}Intermediate phosphonic acid AR was used in the coupling in Step 3 instead of Intermediate B. After Step 3, bromotrimethylsilane was employed to deprotect the phosphonate, where the reaction was run at 40° C. in DCM for 16 h and the final compound was purified by reverse phase chromatography.
^{d}The product of Step 3 was further deprotected with 80% aq. AcOH for 3 h at 50° C. and the final product was purified by reverse phase flash chromatography.

### Example 4 (Method 3). Synthesis of 2-[[(5S,8S,10aR)-8-[[(1S)-3-Carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonylphosphonic acid (I-35)

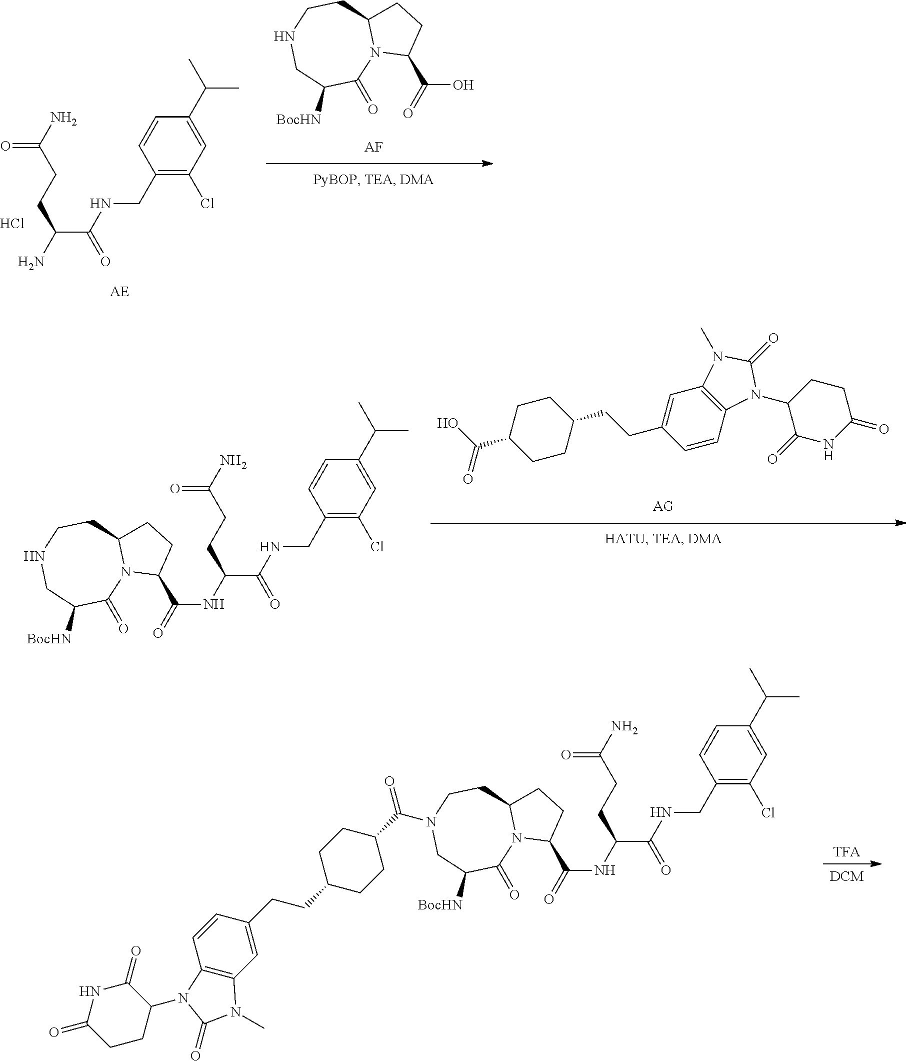

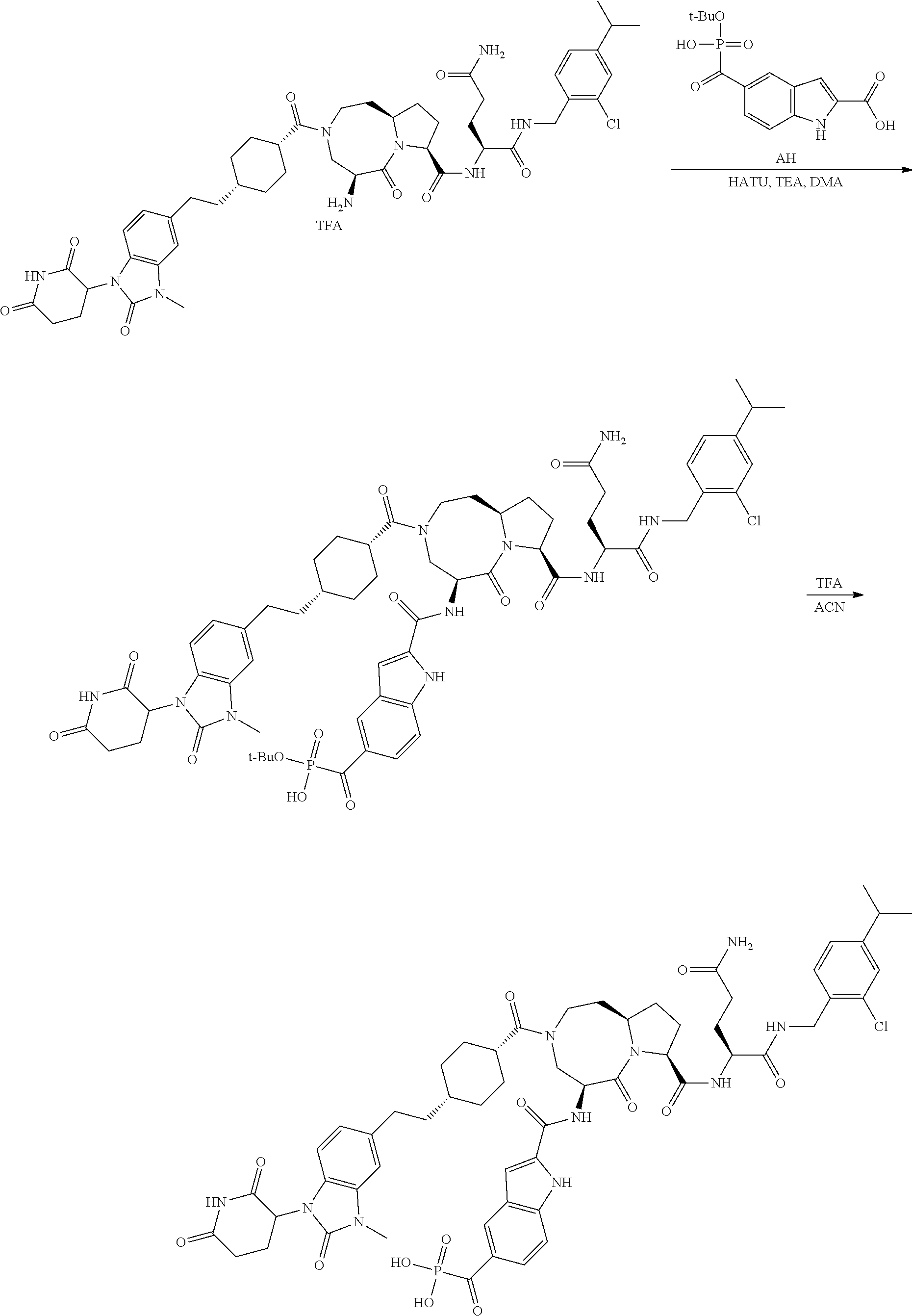

Step 1—Tert-butyl N-[(5S,8S,10aR)-8-[[(1S)-3-carbamoyl-1-[[(2-chloro-4-isopropylphenyl)methyl]carbamoyl]propyl]carbamoyl]-6-oxo-octahydro-1H-pyrrolo[1,2-a][1,5]diazocin-5-yl]carbamate. To a stirred solution of (2S)-2-amino-N-[(2-chloro-4-isopropylphenyl)methyl]pentanediamide hydrochloride (351.06 mg, 1.01 mmol, Intermediate AE) and (5S,8S,10aR)-5-[(tert-butoxycarbonyl)amino]-6-oxo-octahydro-1H-pyrrolo [1,2-a][1,5]diazocine-8-carboxylic acid (300.00 mg, 0.92 mmol, Intermediate AF) in DMA (5.00 mL) were added TEA (278.18 mg, 2.75 mmol) and HATU (418.11 mg, 1.10 mmol) in turns at 0° C. under nitrogen atmosphere. The resulting mixture was stirred for 1 h at rt under nitrogen atmosphere. On completion, the residue was purified by reverse flash chromatography (column, Welflash™ C18-1,20-40 um, 120 g; Eluent A: Water (plus 10 mmol/L FA); Eluent B: ACN; Gradient 30% to 60% B in 30 min; Flow rate: 60 mL/min; Detector: 220/254 nm; desired fractions were collected at 43% B) and concentrated under reduced pressure to afford the title compound (380 mg, 67% yield) as a white solid. ¹H NMR (300 MHz, DMSO-d₆) δ 8.99 (d, J=8.2 Hz, 1H), 7.90-7.86 (m, 1H), 7.32-7.24 (m, 2H), 7.24-7.09 (m, 2H), 6.76-6.72 (m, 2H), 4.74-4.65 (m, 1H), 4.43-4.21 (m, 3H), 4.19-4.12 (m, 1H), 3.19-3.14 (m, 1H), 2.92-2.80 (m, 3H), 2.72-2.56 (m, 2H), 2.41-2.22 (m, 1H), 2.12-2.01 (m, 6H), 1.99-1.64 (m, 3H), 1.36 (s, 9H), 1.18 (d, J=6.9 Hz, 6H); LC/MS (ESI, m/z): [(M+H)]⁺=621.3.

Step 2—Tert-butyl N-[(5S,8S,10aR)-8-[[(1S)-3-carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamate. To a stirred solution of tert-butyl N-[(5S,8S,10aR)-8-[[(1S)-3-carbamoyl-1-[[(2-chloro-4-isopropylphenyl)methyl]carbamoyl]propyl]carbamoyl]-6-oxo-octahydro-1H-pyrrolo[1,2-a][1,5]diazocin-5-yl]carbamate (205.07 mg, 0.33 mmol) and (1s,4s)-4-[2-[1-(2,6-dioxopiperidin-3-yl)-3-methyl-2-oxo-1,3-benzodiazol-5-yl]ethyl]cyclohexane-1-carboxylic acid (130.00 mg, 0.31 mmol, Intermediate AG) in DMA (6.00 mL) were added TEA (95.45 mg, 0.94 mmol) and HATU (143.46 mg, 0.38 mmol) in turns at 0° C. under nitrogen atmosphere. The resulting mixture was stirred for 1 h at room temperature under nitrogen atmosphere. On completion, the residue was purified by reverse phase flash chromatography (Column: WelFlash™ C18-I, 20-40 m, 120 g; Eluent A: Water (plus 10 mmol/L FA); Eluent B: ACN; Gradient: 35%-55% B in 30 min; Flow rate: 50 mL/min; Detector: 220/254 nm; desired fractions were collected at 43% B) and concentrated under reduced pressure to afford the title compound (190 mg, 59%) as a white solid. ¹H NMR (300 MHz, DMSO-d₆) δ 11.06 (s, 1H), 8.36-8.18 (m, 2H), 7.31-7.10 (m, 4H), 7.10-6.94 (m, 2H), 6.87 (d, J=8.0 Hz, 1H), 6.72 (s, 1H), 6.52 (d, J=6.9 Hz, 1H), 5.33 (dd, J=12.8, 5.4 Hz, 1H), 4.54-4.32 (m, 2H), 4.32-4.18 (m, 3H), 4.15-4.02 (m, 1H), 3.77-3.62 (m, 1H), 3.60-3.41 (m, 4H), 3.32 (s, 3H), 3.26-3.02 (m, 1H), 2.98-2.79 (m, 3H), 2.79-2.54 (m, 5H), 2.17-2.07 (m, 3H), 2.05-1.45 (m, 16H), 1.38 (s, 9H), 1.17 (d, J=6.8 Hz, 6H); LC/MS (ESI, m/z): [(M+H)]⁺=1016.4.

Step 3—(2S)-2-[[(5S,8S,10aR)-5-Amino-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-8-yl]formamido]-N-[(2-chloro-4-isopropylphenyl)methyl]pentanediamide trifluoroacetate. To a stirred solution of tert-butyl N-[(5S,8S,10aR)-8-[[(1S)-3-carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamate (180.00 mg) in DCM (0.70 mL) was added TFA (5.60 mL) dropwise at rt under nitrogen atmosphere and the mixture was stirred for 2 h. On completion, the mixture was concentrated under reduced pressure to afford the title compound (170 mg, 94% yield) as a brown solid. ¹H NMR (300 MHz, DMSO-d₆) δ 11.06 (s, 1H), 8.36-8.18 (m, 5H), 7.31-7.10 (m, 4H), 7.10-6.94 (m, 2H), 6.87 (d, J=8.0 Hz, 1H), 6.72 (s, 1H), 6.52 (d, J=6.9 Hz, 1H), 5.33 (dd, J=12.8, 5.4 Hz, 1H), 4.54-4.32 (m, 2H), 4.32-4.18 (m, 3H), 4.15-4.02 (m, 1H), 3.77-3.62 (m, 1H), 3.60-3.41 (m, 4H), 3.32 (s, 3H), 3.26-3.02 (m, 1H), 2.98-2.79 (m, 3H), 2.79-2.54 (m, 5H), 2.17-2.07 (m, 3H), 2.05-1.45 (m, 16H), 1.17 (d, J=6.8 Hz, 6H); LC/MS (ESI, m/z): [(M+H-TFA)]=916.4.

Step 4—2-[[(5S,8S,10aR)-8-[[(1S)-3-Carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonyl(tert-butoxy)phosphinic acid. To a stirred solution of (2S)-2-[[(5S,8S,10aR)-5-amino-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-8-yl]formamido]-N-[(2-chloro-4-isopropylphenyl)methyl]pentanediamide trifluoroacetate (170.00 mg, 0.17 mmol) and 5-[[tert-butoxy(hydroxy)phosphoryl]carbonyl]-1H-indole-2-carboxylic acid (57.23 mg, 0.18 mmol, Intermediate AH) in DMA (5.00 mL) were added TEA (50.87 mg, 0.50 mmol) and HATU (76.46 mg, 0.20 mmol) in turns at 0° C. under nitrogen atmosphere. The resulting mixture was stirred for 1 h at rt under nitrogen atmosphere. On completion, the residue was purified by reverse flash chromatography (column, Welflash™ C18-1,20-40 um, 120 g; Eluent A: Water (plus 10 mmol/L FA); Eluent B: ACN; Gradient 20% to 50% B in 30 min; Flow rate: 50 mL/min; Detector: 220/254 nm; desired fractions were collected at 43% B) and concentrated under reduced pressure to afford the title compound (120 mg, 71% yield) as a white solid. ¹H NMR (300 MHz, DMSO-d₆) δ 12.12-11.93 (m, 1H), 11.10 (s, 1H), 8.85 (s, 1H), 8.46-8.23 (m, 3H), 8.00 (d, J=8.8 Hz, 1H), 7.52 (d, J=8.8 Hz, 1H), 7.45-7.37 (m, 1H), 7.35-7.20 (m, 3H), 7.19-7.14 (m, 1H), 7.08-6.97 (m, 2H), 6.93-6.85 (m, 1H), 6.83-6.72 (m, 1H), 5.42-5.28 (m, 1H), 5.03-4.92 (m, 1H), 4.52-4.44 (m, 1H), 4.36-4.12 (m, 5H), 4.03-3.63 (m, 3H), 3.35 (s, 3H), 3.11-2.80 (m, 3H), 2.80-2.56 (m, 4H), 2.27-2.09 (m, 3H), 2.09-1.90 (m, 4H), 1.90-1.50 (m, 14H), 1.42 (s, 9H), 1.29-1.24 (m, 1H), 1.19 (d, J=6.9 Hz, 6H); LC/MS (ESI, m/z): [(M+H)]⁺=1224.6.

Step 5—2-[[(5S,8S, 10aR)-8-[[(1S)-3-Carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonylphosphonic acid. To a stirred solution of 2-[[(5S,8S,10aR)-8-[[(1S)-3-carbamoyl-1-[[(2-chloro-4-isopropylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonyl(tert-butoxy)phosphinic acid (120.00 mg, 0.098 mmol) in ACN (4.00 mL) was added TFA (0.50 mL, 6.73 mmol) dropwise at rt under nitrogen atmosphere and the reaction mixture was stirred for 1 h at rt under nitrogen atmosphere. On completion, the resulting mixture was concentrated under reduced pressure. The residue was purified by reverse flash chromatography (column, Welflash™ C18-1,20-40 um, 120 g; EluentA: Water (plus 10 mmol/1 FA); Eluent B: ACN; Gradient 20% to 50% B in 30 min; Flow rate: 50 mL/min; Detector: 220/254 nm; desired fractions were collected at 39% B) and concentrated under reduced pressure to afford the title compound (55.2 mg, 45% yield) as a white solid. ¹H NMR (300 MHz, DMSO-d₆) δ 12.12-11.93 (m, 1H), 11.10 (s, 1H), 8.85 (s, 1H), 8.46-8.23 (m, 3H), 8.00 (d, J=8.8 Hz, 1H), 7.52 (d, J=8.8 Hz, 1H), 7.45-7.37 (m, 1H), 7.35-7.20 (m, 3H), 7.19-7.14 (m, 1H), 7.08-6.97 (m, 2H), 6.93-6.85 (m, 1H), 6.83-6.72 (m, 1H), 5.42-5.28 (m, 1H), 5.03-4.92 (m, 1H), 4.52-4.44 (m, 1H), 4.36-4.12 (m, 5H), 4.03-3.63 (m, 3H), 3.35 (s, 3H), 3.11-2.80 (m, 3H), 2.80-2.56 (m, 4H), 2.27-2.09 (m, 3H), 2.09-1.90 (m, 4H), 1.90-1.37 (m, 15H), 1.29-1.24 (m, 1H), 1.19 (d, J=6.9 Hz, 6H). LC/MS (ESI, m/z): [(M+H)]⁺=1167.1.

TABLE 5
Compounds synthesized via Method 3, where the corresponding amines and acids were
coupled in Step 1.

| | | | LC/MS | |
|---|---|---|---|---|
| | Step 1 | Step 1 | (ESI, m/z) | |
| I-# | Amine | Acid | (M + H)⁺ | 1H NMR (400 MHZ, DMSO-d6) δ |
| I-23^{f} | CF | H | 1283.8 | 12.12-11.75 (m, 1H), 9.00 (s, 1H), 8.93-8.78 (m, 1H), 8.49 |
| | | | | (d, J = 6.9 Hz, 1H), 8.35 (d, J = 7.7 Hz, 1H), 8.15-7.94 (m, |
| | | | | 2H), 7.82 (d, J = 8.8 Hz, 1H), 7.47-7.35 (m, 6H), 7.31-7.22 |
| | | | | (m, 1H), 6.88-6.78 (m, 2H), 6.71-6.58 (m, 1H), 5.09-4.89 |
| | | | | (m, 2H), 4.50-4.29 (m, 4H), 4.27-4.14 (m, 2H), 4.07-3.82 |
| | | | | (m, 6H), 3.78-3.64 (m, 3H), 3.43-3.34 (m, 3H), 2.47 (s, |
| | | | | 3H), 2.40-2.01 (m, 14H), 1.97-1.60 (m, 7H), 1.59-1.46 (m, |
| | | | | 4H), 1.39 (d, J = 7.0 Hz, 3H), 1.34-1.21 (m, 2H), 0.96 (s, |
| | | | | 9H) |
| I-24^{f} | CX | H | 1272.8 | 12.07-11.71 (m, 1H), 9.05-8.68 (m, 2H), 8.54-8.36 (m, 1H), |
| | | | | 8.10-7.76 (m, 2H), 7.58-7.12 (m, 10H), 7.12-6.84 (m, 2H), |
| | | | | 6.82-6.71 (m, 1H), 5.10-4.80 (m, 2H), 4.58-4.21 (m, 5H), |
| | | | | 4.11-3.75 (m, 8H), 3.53-3.10 (m, 7H), 2.82-2.60 (m, 2H), |
| | | | | 2.60-2.39 (m, 3H), 2.36-2.21 (m, 4H), 2.20 (s, 3H), 2.19- |
| | | | | 2.10 (m, 6H), 1.66-1.20 (m, 8H), 0.95 (s, 9H) |
| I-28^{c} | AK | A | 1029.5 | 11.93 (s, 1H), 11.08 (s, 1H), 8.82 (s, 1H), 8.67 (d, J = 7.0 |
| | | | | Hz, 1H), 8.07-7.90 (m, 2H), 7.52-7.35 (m, 2H), 7.31-7.10 |
| | | | | (m, 2H), 7.06-6.94 (m, 3H), 6.91-6.82 (m, 2H), 6.75 (s, |
| | | | | 1H), 5.34 (dd, J = 12.6, 5.4 Hz, 1H), 5.07-4.91 (m, 1H), |
| | | | | 4.35-4.27 (m, 1H), 4.15-3.86 (m, 3H), 3.32 (s, 3H), 2.96- |
| | | | | 2.84 (m, 1H), 2.76-2.56 (m, 4H), 2.24-2.15 (m, 2H), 2.12- |
| | | | | 1.94 (m, 4H), 1.94-1.76 (m, 7H), 1.74-1.53 (m, 10H), 1.41- |
| | | | | 1.31 (m, 3H), 1.28-1.17 (m, 2H) |
| I-30 | BC | AF | 1151.7 | 12.22-11.96 (m, 1H), 11.08 (s, 1H), 8.85-8.74 (m, 1H), |
| | | | | 8.49-8.20 (m, 3H), 7.96 (d, J = 8.7 Hz, 1H), 7.53 (d, J = 8.8 |
| | | | | Hz, 1H), 7.44 (d, J = 7.3 Hz, 1H), 7.32-7.18 (m, 2H), 7.10- |
| | | | | 6.94 (m, 4H), 6.89 (d, J = 8.0 Hz, 1H), 6.75 (s, 1H), 5.34 |
| | | | | (dd, J = 12.9, 5.3 Hz, 1H), 5.02-4.79 (m, 1H), 4.49-4.41 (m, |
| | | | | 1H), 4.37-4.08 (m, 5H), 4.03-3.89 (m, 2H), 3.87-3.71 (m, |
| | | | | 3H), 3.33 (s, 3H), 3.18-2.82 (m, 2H), 2.77-2.57 (m, 6H), |
| | | | | 2.28-2.06 (m, 2H), 2.06-1.85 (m, 6H), 1.84-1.48 (m, 12H), |
| | | | | 1.29-1.22 (m, 1H), 1.19 (d, J = 6.9 Hz, 6 H) |
| I-33 | AJ | AF | 1167.5 | 12.07 (s, 1H), 11.09 (s, 1H), 8.82 (s, 1H), 8.50-8.21 (m, |
| | | | | 3H), 7.98 (d, J = 8.8 Hz, 1H), 7.54 (d, J = 8.8 Hz, 1H), 7.46- |
| | | | | 7.38 (m, 1H), 7.36-7.24 (m, 3H), 7.20 (d, J = 8.1 Hz, 1H), |
| | | | | 7.09-6.98 (m, 2H), 6.90 (d, J = 8.1 Hz, 1H), 6.80 (s, 1H), |
| | | | | 5.35 (dd, J = 12.7, 5.4 Hz, 1H), 5.04-4.82 (m, 1H), 4.51- |
| | | | | 4.42 (m, 1H), 4.36-4.18 (m, 4H), 4.03-3.92 (m, 1H), |
| | | | | 3.843.71 (m, 1H), 3.35 (s, 3H), 3.31-3.22 (m, 4H), 3.07- |
| | | | | 2.85 (m, 3H), 2.81-2.56 (m, 4H), 2.25-2.09 (m, 3H), 2.08- |
| | | | | 1.38 (m, 18H), 1.27-1.23 (m, 1H), 1.19 (d, J = 6.9 Hz, 6H) |
| I-34 | AI | AF | 1151.5 | 11.97 (s, 1H), 11.02 (s, 1H), 8.90 (s, 1H), 8.47-8.24 (m, |
| | | | | 3H), 7.98 (d, J = 8.9 Hz, 1H), 7.48-7.43 (m, 2H), 7.40-7.15 |
| | | | | (m, 4H), 7.10-6.93 (m, 4H), 6.95-6.74 (m, 2H), 5.35 (dd, J = |
| | | | | 12.7, 5.4 Hz, 1H), 5.01-4.98 (m, 1H), 4.47 (t, J = 8.4 Hz, |
| | | | | 1H), 4.26-4.21 (m, 4H), 3.96-3.67 (m, 5H), 3.29-3.25 (m, |
| | | | | 2H), 3.04-2.81 (m, 4H), 2.77-2.59 (m, 4H), 2.22-2.10 (m, |
| | | | | 3H), 2.01-1.01 (m, 18H), 1.19 (d, J = 6.9 Hz, 6H) |
| I-37^{g} | DW | AF | 1091.4 | 12.05-11.91 (m, 1H), 11.10 (s, 1H), 9.04 (d, J = 7.7 Hz, 1H), |
| | | | | 8.91-8.85 (m, 1H), 8.50-8.48 (m, 1H), 8.13-7.96 (m, 3H), |
| | | | | 7.53-7.42 (m, 4H), 7.30 (s, 1H), 7.08-6.99 (m, 2H), 6.90- |
| | | | | 6.85 (m, 1H), 5.37-5.33 (m, 1H), 5.24-5.15 (m, 1H), 5.09- |
| | | | | 4.97 (m, 1H), 4.51 (t, J = 8.3 Hz, 1H), 4.24-4.14 (m, 3H), |
| | | | | 3.98-3.75 (m, 3H), 3.19 (s, 3H), 3.05-2.85 (m, 2H), 2.77- |
| | | | | 2.58 (m, 4H), 2.37-2.25 (m, 5H), 2.14-1.91 (m, 6H), 1.80- |
| | | | | 1.55 (m, 13H) |
| I-39^{e} | AL | — | 1035.5 | 12.20-11.99 (m, 1H), 11.11-11.06 (m, 1H), 8.87-8.79 (m, |
| | | | | 1H), 8.57-8.46 (m, 3H), 8.01-7.93 (m, 1H), 7.83-7.73 (m, |
| | | | | 1H), 7.55 (d, J = 8.7 Hz, 1H), 7.49-7.43 (m, 1H), 7.34-7.24 |
| | | | | (m, 2H), 7.22 (s, 1H), 7.08-6.98 (m, 2H), 6.92-6.85 (m, |
| | | | | 1H), 6.75 (s, 1H), 5.39-5.30 (m, 1H), 5.04-4.96 (m, 1H), |
| | | | | 4.90-4.80 (m, 1H), 4.49 (t, J = 8.4 Hz, 1H), 4.20-4.16 (m, |
| | | | | 1H), 3.98-3.90 (m, 1H), 3.77-3.69 (m, 1H), 3.60-3.49 (m, |
| | | | | 1H), 3.42-3.37 (m, 1 H), 3.34 (s, 3H), 3.30-3.24 (m, 2 H), |
| | | | | 3.04-2.99 (m, 1H), 2.96-2.84 (m, 1H), 2.76-2.67 (m, 1H), |
| | | | | 2.66-2.58 (m, 3H), 2.24-1.86 (m, 8H), 1.80-1.46 (m, 14H) |
| I-50^{c} | (4R)-4- | AM | 972.5 | 12.18-12.00 (m, 1H), 11.09-11.05 (m, 1H), 8.87-8.78 (m, |
| | aminopent- | | | 1H), 8.53 (d, J = 7.1 Hz, 1H), 7.99-7.91 (m, 1H), 7.88-7.79 |
| | anamide | | | (m, 1H), 7.58-7.49 (m, 1H), 7.48-7.43 (m, 1H), 7.25-7.17 |
| | | | | (m, 1H), 7.03-6.93 (m, 2H), 6.88-6.76 (m, 1H), 6.75-6.62 |
| | | | | (m, 1H), 5.38-5.28 (m, 1H), 5.01-4.87 (m, 1H), 4.33-4.25 |
| | | | | (m, 1H), 4.20-4.11 (m, 1H), 3.97-3.89 (m, 1H), 3.83-3.64 |
| | | | | (m, 2H), 3.40-3.30 (m, 5H), 3.28 (s, 3H), 2.92-2.83 (m, |
| | | | | 1H), 2.71-2.54 (m, 5H), 2.18-2.04 (m, 3H), 2.03-1.94 (m, |
| | | | | 3H), 1.89-1.77 (m, 2H), 1.75-1.63 (m, 3H), 1.62-1.52 (m, |
| | | | | 2H), 1.51-1.29 (m, 8H), 1.06-0.99 (m, 3H) |
| I-51^{c} | AN | AM | 1142.6 | 12.19-11.98 (m, 1H), 11.07 (s, 1H), 8.84-8.79 (m, 1H), |
| | | | | 8.55-8.50 (m, 1H), 8.09 (t, J = 7.7 Hz, 1H), 7.99-7.91 (m, |
| | | | | 1H), 7.61-7.48 (m, 3H), 7.47-7.41 (m, 2H), 7.31-7.19 (m, |
| | | | | 2H), 7.03-6.93 (m, 2H), 6.87-6.75 (m, 2H), 5.38-5.27 (m, |
| | | | | 1H), 5.02-4.90 (m, 1H), 4.42-4.33 (m, 1H), 4.23-4.13 (m, |
| | | | | 1H), 4.11-3.88 (m, 4H), 3.83-3.52 (m, 2H), 3.36-3.29 (m, |
| | | | | 5H), 3.25 (s, 3H), 3.22 (s 3H), 2.95-2.83 (m, 1H), 2.75-2.54 |
| | | | | (m, 5H), 2.27-2.08 (m, 3H), 2.07-1.94 (m, 3H), 1.92-1.82 |
| | | | | (m, 2H), 1.80-1.63 (m, 4H), 1.52-1.28 (m, 7H) |
| I-59^{f} | BL | BK | 1133.6 | 12.17-11.97 (m, 1H), 11.09 (s, 1H), 8.82 (s, 1H), 8.45-8.39 |
| | | | | (m, 1H), 8.38-8.29 (m, 1H), 8.28-8.18 (m, 1H), 8.01-7.94 |
| | | | | (m, 1H), 7.53 (d, J = 8.7 Hz, 1H), 7.48-7.41 (m, 1H), 7.29- |
| | | | | 7.20 (m, 1H), 7.17 (s, 4H), 7.08-6.98 (m, 2H), 6.95-6.85 |
| | | | | (m, 1H), 6.79 (s, 1H), 5.39-5.30 (m, 1H), 5.05-4.93 (m, |
| | | | | 1H), 4.91-4.75 (m, 1H), 4.49-4.40 (m, 1H), 4.28-4.16 (m, |
| | | | | 4H), 3.98-3.91 (m, 1H), 3.84-3.71(m, 2H), 3.48-3.46 (m, |
| | | | | 2H), 3.34 (s, 3H), 3.07-2.98 (m, 1H), 2.93-2.79 (m, 2H), |
| | | | | 2.66-2.58 (m, 3H), 2.23-2.04 (m, 5H), 2.04-1.85 (m, 4H), |
| | | | | 1.83-1.73 (m, 3H), 1.72-1.46 (m, 11H), 1.22-1.13 (m, 6H) |
| I-70^{c, d} | AP | AQ | 1195.6^{b} | 12.10 (d, J = 47.7 Hz, 1H), 11.09 (s, 1H), 8.84-8.82 (m, |
| | | | | 2H), 8.59-8.50 (m, 2H), 8.38-8.23 (m, 1H), 7.98 (d, J = 8.9 |
| | | | | Hz, 1H), 7.83-7.73 (m, 2H), 7.60-7.49 (m, 3H), 7.46 (d, J = |
| | | | | 2.1 Hz, 1H), 7.30-7.26 (m, 1H), 7.05-6.93 (m, 2H), 6.90- |
| | | | | 6.70 (m, 2H), 5.34 (dd, J = 12.7, 5.1 Hz, 1H), 5.04-4.87 (m, |
| | | | | 1H), 4.51-4.41 (m, 3H), 4.31-4.19 (m, 2H), 4.09-3.94 (m, |
| | | | | 1H), 3.87-3.79 (m, 1H), 3.46-3.32 (m, 4H), 3.30 (m, 3H), |
| | | | | 2.96-2.87 (m, 1H), 2.80-2.55 (m, 6H), 2.31-2.09 (m, 4H), |
| | | | | 2.09-1.60 (m, 8H), 1.46 (s, 9H), 1.15 (d, J = 6.8 Hz, 6H) |

ªIn Step 2, acid Intermediate AG was coupled unless otherwise noted. In Step 4, phosphonic acid Intermediate AH was coupled unless otherwise noted. Step 1 was run anywhere from 1-2 h at rt. Step 2 was run anywhere from 1-4 h at rt. Step 3 was run anywhere from 1-2 h at rt. Dioxane, EtOAc, THF or DCM used as the solvent; HCl in dioxane could also be used for the deprotection in place of TFA. Step 4 was run anywhere from 1-3 h at rt. PyBOP, TEA, in DMA could also be used for the coupling. Step 5 was run anywhere from 1-2 h at rt; ACN or DCM were used as the solvent.
^{b}[(M − H)]⁻ LCMS ion reported not [(M + H)]⁺.
^{c}The coupling of Intermediate AG in Step 2 was skipped for synthesis of this compound.
^{d}For Step 5 deprotection, bromotrimethylsilane was employed to deprotect the phosphonate, where the reaction was run at 40° C. in DCM for 16 h, and the final compound was purified by reverse phase chromatography.
^{e}Step 1 was skipped for this compound and amine Intermediate AL was used in Step 2 to couple with acid Intermediate AG.
^{f}Step 1 was skipped for this compound, the amine and acid listed are for Step 2 coupling, not Intermediate AG.
^{g}Step 2 utilized Intermediate Acid BK instead of Intermediat AG.

### Example 5. Synthesis of 2-[[(2S,11S)-2-[[(1S)-3-carbamoyl-1-[[(4-methanesulfonylphenyl)methyl]carbamoyl]propyl]carbamoyl]-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13]]trideca-4(13),5,7-trien-11-yl]carbamoyl]-1H-indole-5-carbonylphosphonic acid (I-57)

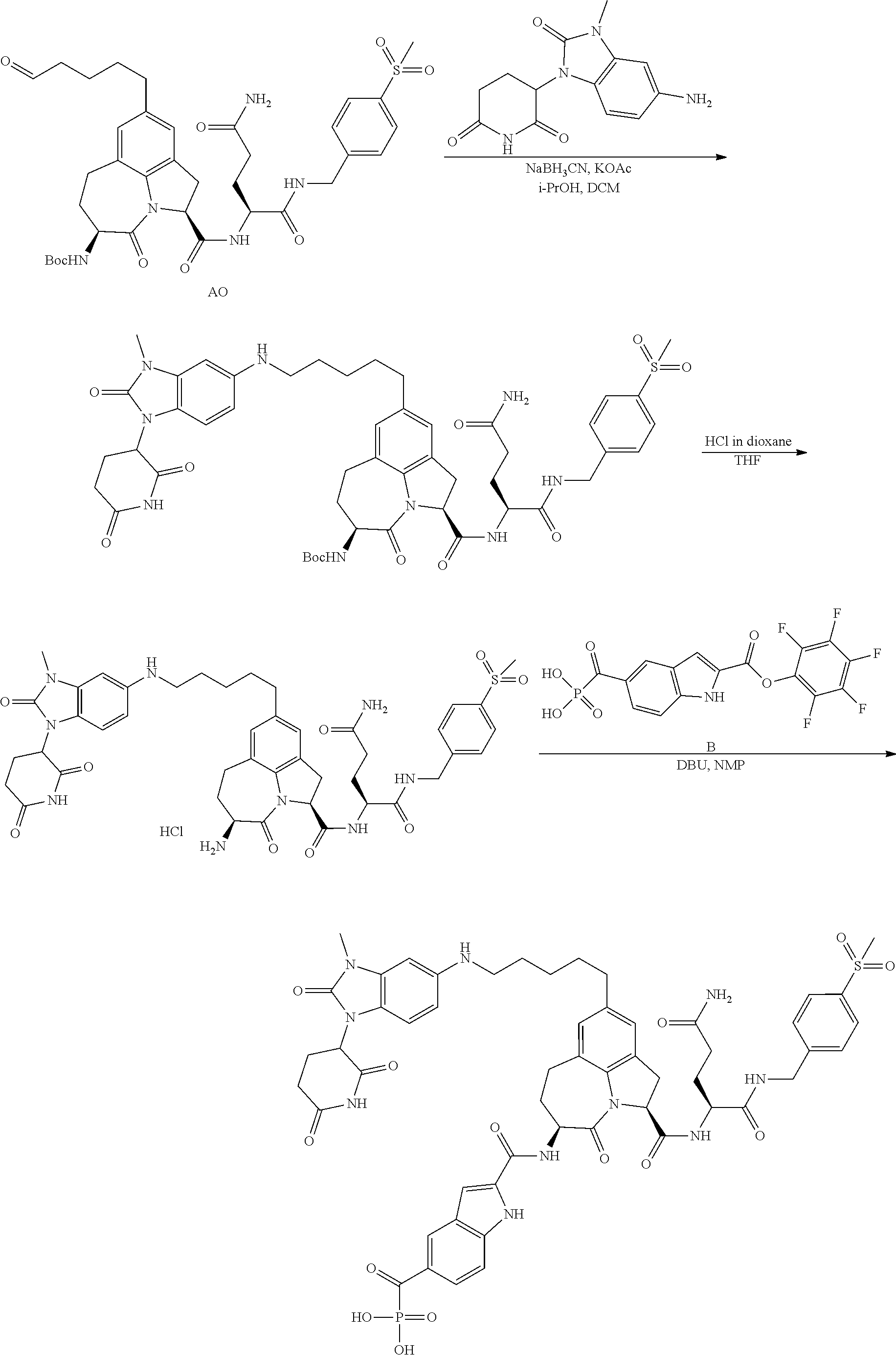

Step 1—tert-butyl N-[(2S,11S)-2-[[(1S)-3-carbamoyl-1-[[(4-methanesulfonylphenyl)methyl]carbamoyl]propyl]carbamoyl]-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13]]trideca-4(13),5,7-trien-11-yl]carbamate. To a stirred mixture of tert-butyl N-[(2S,11S)-2-[[(1S)-3-carbamoyl-1-[[(4-methanesulfonylphenyl)methyl]carbamoyl]propyl]carbamoyl]-12-oxo-6-(5-oxopentyl)-1-azatricyclo[6.4.1.0\hat{ }[4,13]]trideca-4(13),5,7-trien-11-yl]carbamate (60.00 mg, 0.083 mmol) and KOAc (24.34 mg, 0.25 mmol, Intermediate AO) in i-PrOH (4.00 mL) and DCM (1.00 mL) was added 3-(5-amino-3-methyl-2-oxo-1,3-benzodiazol-1-yl)piperidine-2,6-dione (27.21 mg, 0.10 mmol) in portions at rt under nitrogen atmosphere and the mixture was stirred for 3 h. On completion, the mixture was concentrated under reduced pressure. The residue was purified by reverse flash chromatography (column, C¹⁸ silica gel; mobile phase, MeCN in water (10 mmol/L FA), 25% to 40% gradient in 20 min; detector, UV 220 nm) to afford the title compound (53 mg, 65% yield) as a white solid. ¹H NMR (300 MHz, Chloroform-d) δ 9.01 (d, J=16.4 Hz, 1H), 7.91-7.87 (m, 2H), 7.77-7.55 (m, 1H), 7.50-7.46 (m, 2H), 6.86-6.82 (m, 3H), 6.69-6.60 (m, 1H), 6.43-6.32 (m, 2H), 5.96-5.90 (m, 1H), 5.52-5.48 (m, 1H), 5.27-5.08 (m, 2H), 4.52-4.48 (m, 3H), 4.34-4.30 (m, 1H), 3.40-3.36 (m, 3H), 3.23-3.07 (m, 4H), 3.06 (s, 3H), 2.97-2.69 (m, 2H), 2.67-2.51 (m, 2H), 2.43-2.29 (m, 2H), 2.28-2.14 (m, 3H), 2.13-1.97 (m, 2H), 1.87-1.56 (m, 6H), 1.48 (s, 9H), 1.44-1.40 (m, 2H), 1.31-1.19 (m, 3H); LC/MS (ESI, m z): [(M+1)]⁺=984.4.

Step 2—Synthesis of (2S)-2-[[(2S,11S)-11-amino-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13]]trideca-4(13),5,7-trien-2-yl]formamido]-N-[(4-methanesulfonylphenyl)methyl]pentanediamide hydrochloride. To a stirred solution of tert-butyl N-[(2S,11S)-2-[[(1S)-3-carbamoyl-1-[[(4-methanesulfonylphenyl)methyl]carbamoyl]propyl]carbamoyl]-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13]]trideca-4(13),5,7-trien-11-yl]carbamate (53.00 mg, 0.054 mmol) in THF (5 mL) was added HCl (gas) in 1,4-dioxane (5 mL) dropwise at rt under nitrogen atmosphere and the reaction mixture was stirred for 1 h. On completion, the resulting mixture was concentrated under reduced pressure to afford the title compound (48 mg, 97% yield) as a white solid. ¹H NMR (300 MHz, Chloroform-d) δ 11.13 (s, 1H), 8.61 (s, 1H), 8.48-8.40 (m, 4H), 7.83 (d, J=8.4 Hz, 2H), 7.46 (d, J=8.1 Hz, 2H), 7.39-7.37 (m, 1H), 7.32 (s, 1H), 7.25-7.22 (m, 2H), 6.96 (s, 1H), 6.89 (s, 1H), 6.80 (s, 1H), 5.43-5.39 (m, 1H), 5.17-5.13 (m, 1H), 4.69-4.56 (m, 1H), 4.38-4.34 (m, 2H), 4.27-4.14 (m, 4H), 3.76-3.62 (m, 2H), 3.61-3.57 (m, 1H), 3.57 (s, 3H), 3.52-3.38 (m, 1H), 3.37-3.33 (m, 2H), 3.29-3.22 (m, 1H), 3.18 (s, 3H), 3.13-3.03 (m, 2H), 2.97-2.85 (m, 2H), 2.76-2.57 (m, 2H), 2.17-2.09 (m, 3H), 2.08-1.96 (m, 1H), 1.83-1.70 (m, 1H), 1.61-1.45 (m, 1H), 1.41-1.31 (m, 1H), 1.27-1.15 (m, 1H); LC/MS (ESI, m z): [(M+1)]⁺=884.4.

Step 3—2-[[(2S,11S)-2-[[(1S)-3-carbamoyl-1-[[(4-methanesulfonylphenyl)methyl]carbamoyl]propyl]carbamoyl]-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13]]trideca-4(13),5,7-trien-11-yl]carbamoyl]-1H-indole-5-carbonylphosphonic acid. To a stirred solution of (2S)-2-[[(2S,11S)-11-amino-6-(5-[[1-(2,6-dioxopiperidin-3-yl)-3-methyl-2-oxo-1,3-benzodiazol-5-yl]amino]pentyl)-12-oxo-1-azatricyclo[6.4.1.0\hat{ }[4,13 ]]trideca-4(13),5,7-trien-2-yl]formamido]-N-[(4-methanesulfonylphenyl)methyl]pentanediamide hydrochloride (48.00 mg, 0.052 mmol) and 2-(2,3,4,5,6-pentafluorophenoxycarbonyl)-1H-indole-5-carbonylphosphonic acid (56.74 mg, 0.130 mmol, Intermediate B) in NMP (2 mL) was added DBU (39.69 mg, 0.261 mmol) dropwise at rt under nitrogen atmosphere and the mixture was stirred for 16 h. On completion, the residue was purified by reverse flash chromatography (column, C¹⁸ silica gel; mobile phase, MeCN in water (10 mmol/L FA), 20% to 40% gradient in 25 min; detector, UV 220 nm) to afford the title compound (14 mg, 24% yield) as a white solid. ¹H NMR (300 MHz, DMSO-d₆) δ 12.05 (s, 1H), 11.04 (s, 1H), 8.95 (d, J=7.9 Hz, 1H), 8.85 (s, 1H), 8.57-8.55 (m, 1H), 8.31 (d, J=7.6 Hz, 1H), 7.96 (d, J=8.8 Hz, 1H), 7.86-7.82 (m, 2H), 7.55-7.42 (m, 4H), 7.30 (s, 1H), 7.23-7.11 (m, 1H), 6.97-6.93 (m, 2H), 6.82-6.78 (m, 2H), 6.42-6.40 (m, 1H), 6.32-6.28 (m, 1H), 5.26-5.22 (m, 1H), 5.17-5.13 (m, 1H), 4.69-4.65 (m, 1H), 4.39-4.35 (m, 2H), 4.24-4.20 (m, 1H), 3.27 (s, 3H), 3.20-3.16 (m, 3H), 3.15-3.09 (m, 2H), 3.07-2.97 (m, 4H), 2.95-2.81 (m, 2H), 2.77-2.55 (m, 3H), 2.33-2.19 (m, 2H), 2.14-2.05 (m, 3H), 2.03-1.88 (m, 3H), 1.86-1.75 (m, 1H), 1.72-1.53 (m, 4H), 1.46-1.37 (m, 2H); LC/MS (ESI, m/z): [(M+1)]⁺=1135.5.

### Example 6. Synthesis of 2-[[(2S,11S)-6-[6-[1-(2,6-dioxopiperidin-3-yl)-3-methyl-2-oxo-1,3-benzodiazol-5-yl]hexyl]-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamoyl]-1H-indole-5-carbonylphosphonic acid (I-65)

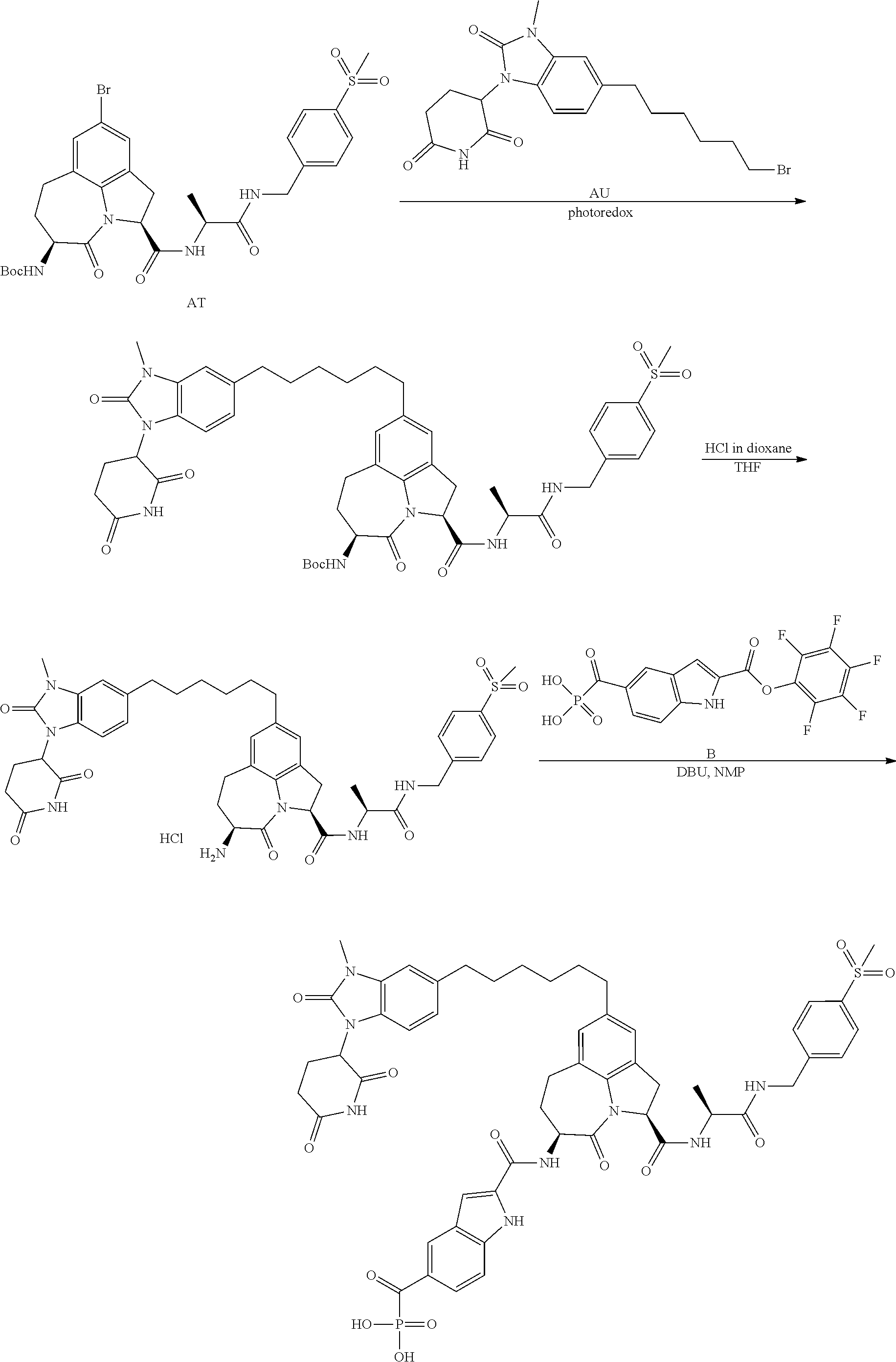

Step 1—tert-butyl N-[(2S,11S)-6-[6-[1-(2,6-dioxopiperidin-3-yl)-3-methyl-2-oxo-1,3-benzodiazol-5-yl]hexyl]-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamate. To a mixture of dtbbpy (4.04 mg, 0.015 mmol) in DME (2.00 mL) was added 1,2-dimethoxyethane dihydrochloride nickel (3.31 mg, 0.015 mmol) at rt and the mixture was bubbled with nitrogen for 10 min. To a mixture of tert-butyl N-[(2S,11S)-6-bromo-2-[[(1S)-1-[[(4-methanesulfonylphenyl)methyl]carbamoyl]ethyl]carbamoyl]-12-oxo-1-azatricyclo[6.4.1.0\hat{ }[4,13 ]]trideca-4(13),5,7-trien-11-yl]carbamate (1.00 g, 1.51 mmol, Intermediate AT) and 3-[5-(6-bromohexyl)-3-methyl-2-oxo-1,3-benzodiazol-1-yl]piperidine-2,6-dione (763.72 mg, 1.81 mmol, Intermediate AU) in DME (15.00 mL) were added tris(trimethylsilyl)silane (374.73 mg, 1.51 mmol), Na₂CO₃ (479.17 mg, 4.52 mmol), IR[DF(CF₃)PPY]₂(DTBPY)PF₆ (16.91 mg, 0.015 mmol) and the catalyst mixture dropwise at rt under nitrogen atmosphere. The final reaction mixture was irradiated with ultraviolet lamp for 16 h at rt. On completion, the reaction mixture was concentrated under reduced pressure. The residue was purified by reverse flash chromatography (column, C¹⁸ silica gel; mobile phase, MeCN in water (0.1% FA), 50% to 70% gradient in 20 min; detector, UV 220 nm) to afford the title compound (812 mg, 58% yield) as a light yellow solid. ¹H NMR (300 MHz, Chloroform-d) δ 8.62-8.58 (m, 1H), 7.88 (d, J=8.1 Hz, 2H), 7.45 (d, J=8.0 Hz, 2H), 7.28-7.25 (m, 1H), 7.16-7.12 (m, 1H), 6.89-6.85 (m, 4H), 6.74 (d, J=8.0 Hz, 1H), 5.84 (d, J=5.9 Hz, 1H), 5.26-4.49 (m, 3H), 4.47-4.40 (m, 1H), 4.32-4.22 (m, 1H), 3.43 (s, 3H), 3.39-3.35 (m, 1H), 3.33-3.20 (m, 1H), 3.05 (s, 3H), 2.89-2.85 (m, 2H), 2.79-2.60 (m, 4H), 2.54-2.50 (m, 2H), 2.45-2.34 (m, 1H), 2.29-2.17 (m, 2H), 2.09-1.98 (m, 1H), 1.96-1.76 (m, 1H), 1.69-1.53 (m, 4H), 1.47 (s, 9H), 1.39-1.35 (m, 3H), 1.30-1.27 (m, 4H); LC/MS (ESI, m z): [(M+1)]⁺=926.4.

Step 2—(2S)-2-[[(2S,11S)-11-amino-6-[6-[1-(2,6-dioxopiperidin-3-yl)-3-methyl-2-oxo-1,3-benzodiazol-5-yl]hexyl]-12-oxo-1-azatricyclo[6.4.1.0\hat{ }[4,13]]trideca-4(13),5,7-trien-2-yl]formamido]-N-[(4-methanesulfonylphenyl)methyl]propanamide hydrochloride. To a stirred solution of tert-butyl N-[(2S,11S)-6-[6-[1-(2,6-dioxopiperidin-3-yl)-3-methyl-2-oxo-1,3-benzodiazol-5-yl]hexyl]-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamate (300.00 mg, 0.32 mmol) in THF (5 mL) was added HCl (gas) in 1,4-dioxane (5 mL) dropwise at rt under nitrogen atmosphere and the reaction mixture was stirred for 2 h. On completion, the resulting mixture was concentrated under reduced pressure to afford the title compound (268 mg, 96% yield) as a light yellow solid. ¹H NMR (300 MHz, DMSO-d₆) δ 11.10 (s, 1H), 8.62-8.58 (m, 1H), 8.50-8.39 (m, 3H), 7.90-7.80 (m, 2H), 7.49-7.45 (m, 2H), 7.07-6.93 (m, 3H), 6.89-6.85 (m, 2H), 5.38-5.34 (m, 1H), 5.20-5.10 (m, 1H), 4.40-4.36 (m, 2H), 4.32-4.25 (m, 1H), 4.22-4.14 (m, 1H), 3.85-3.79 (m, 1H), 3.50-3.30 (m, 1H), 3.34 (s, 3H), 3.22-3.18 (m, 3H), 3.14-3.10 (m, 2H), 2.95-2.91 (m, 2H), 2.80-2.57 (m, 5H), 2.50-2.47 (m, 1H), 2.28-2.18 (m, 1H), 2.09-1.95 (m, 2H), 1.85-1.72 (m, 1H), 1.67-1.51 (m, 3H), 1.41-1.26 (m, 6H); LC/MS (ESI, m z): [(M+1)]⁺=826.3.

Step 3—2-[[(2S,11S)-6-[6-[1-(2,6-dioxopiperidin-3-yl)-3-methyl-2-oxo-1,3-benzodiazol-5-yl]hexyl]-2-[[(1S)-1-[[(4-methanesulfonylphenyl)methyl]carbamoyl]ethyl]carbamoyl]-12-oxo-1-azatricyclo[6.4.1.0\hat{ }[4,13]]trideca-4(13),5,7-trien-11-yl]carbamoyl]-1H-indole-5-carbonylphosphonic acid. To a stirred solution of (2S)-2-[[(2S,11S)-11-amino-6-[6-[1-(2,6-dioxopiperidin-3-yl)-3-methyl-2-oxo-1,3-benzodiazol-5-yl]hexyl]-12-oxo-1-azatricyclo[6.4.1.0\hat{ }[4,13]]trideca-4(13),5,7-trien-2-yl]formamido]-N-[(4-methanesulfonylphenyl)methyl]propanamide hydrochloride (268.00 mg, 0.31 mmol) and 2-(2,3,4,5,6-pentafluorophenoxycarbonyl)-1H-indole-5-carbonylphosphonic acid (270.47 mg, 0.62 mmol, Intermediate B) in NMP (3.00 mL) was added DBU (236.53 mg, 1.55 mmol) dropwise at rt under nitrogen atmosphere and the mixture was stirred for 16 h. On completion, the residue was purified by reverse flash chromatography (column, C¹⁸ silica gel; mobile phase, MeCN in water (10 mmol/L FA), 20% to 40% gradient in 25 min; detector, UV 220 nm) to afford the title compound (96.9 mg, 29% yield) as a white solid. ¹H NMR (300 MHz, DMSO-d₆) δ 12.09 (s, 1H), 11.10 (s, 1H), 8.96-8.92 (m, 1H), 8.85 (s, 1H), 8.55-8.51 (m, 1H), 8.29 (d, J=7.1 Hz, 1H), 7.97 (d, J=8.8 Hz, 1H), 7.89-7.79 (m, 2H), 7.58-7.42 (m, 4H), 7.08-6.98 (m, 2H), 6.97-6.94 (m, 1H), 6.94-6.84 (m, 2H), 5.37-5.33 (m, 1H), 5.15-5.11 (m, 1H), 4.69-4.65 (m, 1H), 4.34-4.30 (m, 3H), 3.52-3.38 (m, 3H), 3.34 (s, 3H), 3.31-3.23 (m, 1H), 3.19 (s, 3H), 3.14-3.04 (m, 2H), 3.03-2.83 (m, 2H), 2.80-2.67 (m, 1H), 2.68-2.60 (m, 4H), 2.33-2.16 (m, 2H), 2.07-1.97 (m, 1H), 1.66-1.53 (m, 4H), 1.38-1.34 (m, 4H), 1.27 (d, J=7.1 Hz, 3H); LC/MS (ESI, m z): [(M−1)]-=1075.4.

### Example 7 (Method 4): Synthesis of (2-{[(5S, 8S, 10aR)-3-acetyl-8-{[(2S)-4-carbamoyl-1-[2-chloro-3-(3-{[(2S)-1-[(2S, 4R)-4-hydroxy-2-{1[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocin-5-yl]carbamoyl}-1-benzothiophen-5-yl)difluoromethylphosphonate (I-84)

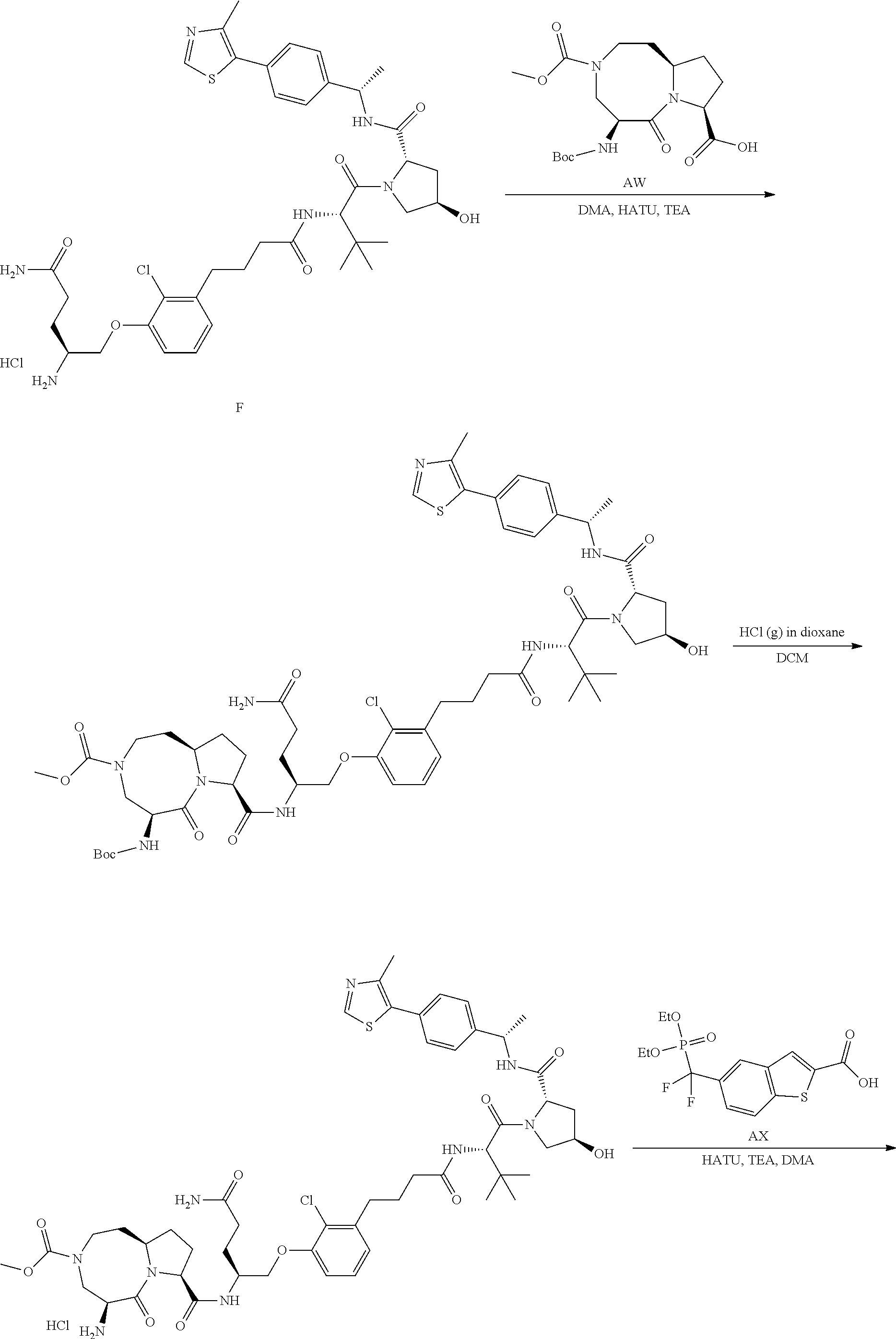

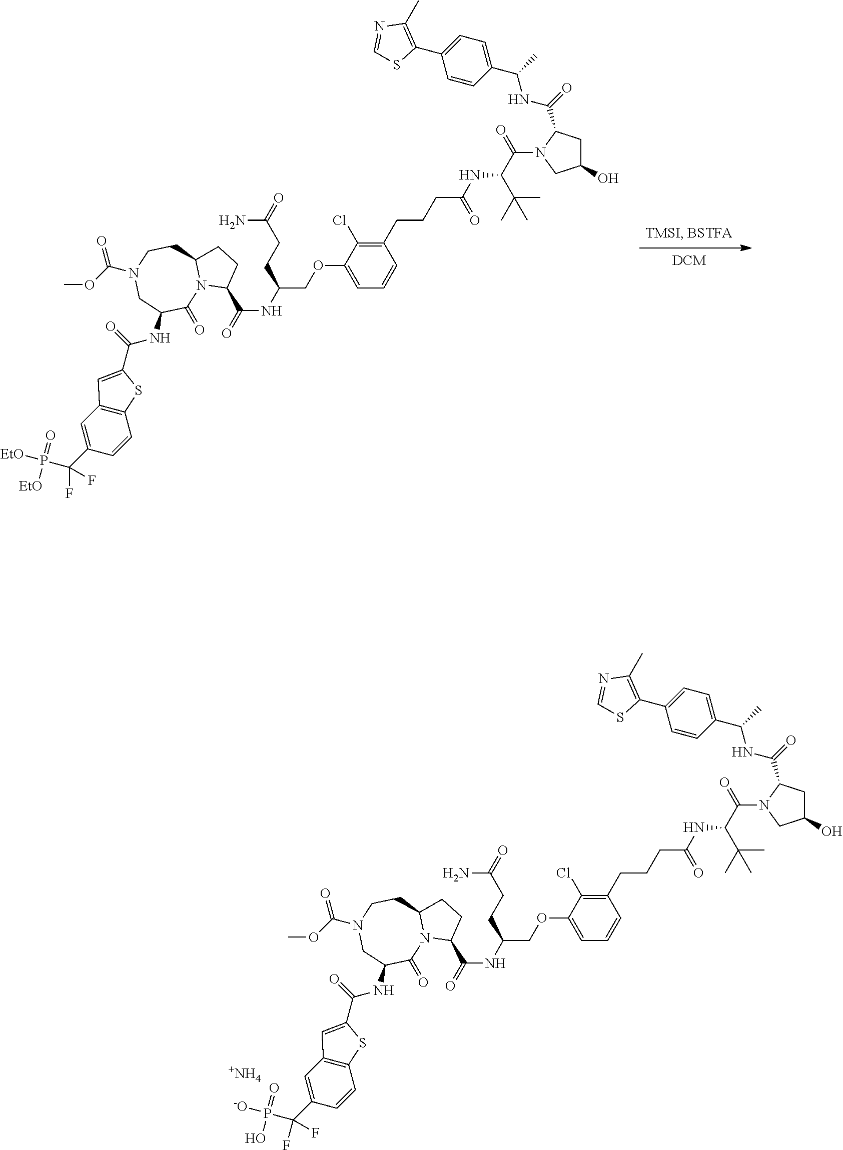

Step 1—Methyl (5S, 8S, 10aR)-5-[(tert-butoxycarbonyl)amino]-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocine-3-carboxylate. To a stirred solution of (2S, 4R)-1-[(2S)-2-(4-{3-[(2S)-2-amino-4-carbamoylbutoxy]-2-chlorophenyl}butanamido)-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (850 mg, 1.07 mmol, Intermediate F) and (5S, 8S, 10aR)-5-[(tert-butoxycarbonyl)amino]-3-(methoxycarbonyl)-6-oxo-octahydropyrrolo[1, 2-a][1,5]diazocine-8-carboxylic acid (413.73 mg, 1.07 mmol, Intermediate AW) in DMA (8.5 mL) were added TEA (543.12 mg, 5.37 mmol) and HATU (489.80 mg, 1.29 mmol) at 0° C. under nitrogen atmosphere. The resulting mixture was stirred for 1 h at rt under N2. On completion, the residue was purified by reverse flash chromatography (column, Welflash™ C18-1, 20-40 um, 330 g; Eluent A: Water (plus 10 mmol/L FA); Eluent B: ACN; Gradient 25% to 55% B in 30 min; Flow rate: 80 mL/min; Detector: 220/254 nm; desired fractions were collected at 43% B) and concentrated under reduced pressure to afford the title compound (730 mg, 61% yield) as a white solid. ¹H NMR (400 MHz, Chloroform-d) δ 8.73 (s, 1H), 7.61 (d, J=7.9 Hz, 1H), 7.43-7.33 (m, 4H), 7.13-7.08 (m, 1H), 7.03-6.94 (m, 1H), 6.88-6.82 (m, 1H), 6.80-6.65 (m, 1H), 6.52 (d, J=9.4 Hz, 1H), 6.35 (d, J=8.6 Hz, 1H), 5.56-5.44 (m, 1H), 5.41-5.35 (m, 1H), 5.9-5.02 (m, 1H), 4.85-4.79 (m, 1H), 4.76-4.66 (m, 1H), 4.66-4.48 (m, 2H), 4.36-4.17 (m, 3H), 4.14-3.96 (m, 3H), 3.92-3.81 (m, 1H), 3.77 (s, 3H), 3.71-3.60 (m, 1H), 3.50-3.38 (m, 1H), 3.25-3.09 (m, 1H), 2.94-2.68 (m, 2H), 2.55 (s, 3H), 2.51-2.33 (m, 4H), 2.29-2.20 (m, 3H), 2.18-2.08 (m, 5H), 2.02-1.80 (m, 4H), 1.51 (d, J=7.1 Hz, 3H), 1.46 (s, 9H), 1.07 (s, 9H); LC/MS (ESI, m/z): [(M+H)]+=1122.5.

Step 2—Methyl (5S, 8S, 10aR)-5-amino-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocine-3-carboxylate hydrochloride. To a stirred solution of methyl (5S, 8S, 10aR)-5-[(tert-butoxycarbonyl)amino]-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocine-3-carboxylate (730 mg, 0.65 mmol) in DCM (14 mL) was added a solution of 4 M HCl (gas) in 1, 4-dioxane (12 mL) dropwise at rt under nitrogen atmosphere. The resulting mixture was then stirred for 2 h at rt under nitrogen atmosphere. On completion, the resulting mixture was concentrated under reduced pressure. The residue was purified by trituration with Et₂O (15 mL). The precipitated solids were collected by filtration and washed with Et2O (2×2 mL) then dried under reduced pressure to afford the title compound (680 mg, 96% yield) as a white solid. ¹H NMR (400 MHz, Methanol-d4) δ 10.04 (s, 1H), 7.60-7.51 (m, 4H), 7.22-7.16 (m, 1H), 6.98-6.88 (m, 2H), 5.04-5.01 (m, 1H), 4.68-4.55 (m, 2H), 4.55-4.37 (m, 3H), 4.33-4.21 (m, 1H), 4.14-4.07 (m, 2H), 4.06-3.95 (m, 1H), 3.94-3.88 (m, 1H), 3.83-3.74 (m, 4H), 3.68 (s, 3H), 3.62-3.46 (m, 2H), 3.44-3.35 (m, 1H), 2.82-2.76 (m, 2H), 2.64 (s, 3H), 2.53-2.43 (m, 2H), 2.41-2.34 (m, 4H), 2.25-2.03 (m, 4H), 2.01-1.82 (m, 4H), 1.53 (d, J=7.0 Hz, 3H), 1.06 (s, 9H); LC/MS (ESI, m/z): [(M+H)]+=1022.4.

Step 3—Methyl (5S, 8S, 10aR)-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-5-{5-[(diethoxyphosphoryl)difluoromethyl]-1-benzothiophene-2-amido}-6-oxo-octahydropyrrolo[1, 2-a][1,5]diazocine-3-carboxylate. To a stirred solution of methyl (5S, 8S, 10aR)-5-amino-8-{[(2S)-4-carbamoyl-1-[2-chloro-3-(3-{[(2S)-1-[(2S, 4R)-4-hydroxy-2-{[(1S)-1-[4-(4-methyl-1,3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocine-3-carboxylate hydrochloride (300 mg, 0.283 mmol) and 5-((diethoxyphosphoryl)difluoromethyl)benzo[b]thiophene-2-carboxylic acid (103.19 mg, 0.283 mmol, Intermediate AX) in DMA (5 mL) were added TEA (143.31 mg, 1.415 mmol) and HATU (129.24 mg, 0.340 mmol) at 0° C. under nitrogen atmosphere. The resulting mixture was then stirred for 1 h at rt under nitrogen atmosphere. On completion, the resulting mixture was concentrated under reduced pressure. The residue was purified by reverse flash chromatography (column, Welflash™ C18-1, 20-40 um, 120 g; Eluent A: Water (plus 10 mmol/L FA); Eluent B: ACN; Gradient 25% to 55% B in 30 min; Flow rate: 65 mL/min; Detector: 220/254 nm; desired fractions were collected at 51% B) and concentrated under reduced pressure to afford the title compound (200 mg, 52% yield) as a white solid. ¹H NMR (300 MHz, Chloroform-d) δ 8.69 (s, 1H), 8.13 (s, 1H), 8.08-8.02 (m, 1H), 8.01-7.79 (m, 2H), 7.65 (d, J=8.6 Hz, 1H), 7.54-7.45 (m, 1H), 77.43-0.34 (m, 4H), 7.31-7.28 (m, 1H), 7.21-6.99 (m, 2H), 6.87-6.69 (m, 3H), 6.65-6.39 (m, 1H), 6.04-5.98 (m, 1H), 5.35-5.30 (m, 1H), 5.15-5.03 (m, 1H), 4.84-4.57 (m, 2H), 4.54-4.49 (m, 1H), 4.43-4.11 (m, 6H), 4.11-3.95 (m, 3H), 3.79 (s, 3H), 3.69-3.53 (m, 2H), 3.42-3.34 (m, 1H), 2.83-2.66 (m, 2H), 2.53 (s, 3H), 2.47-2.29 (m, 4H), 3.01-2.03 (m, 9H), 1.98-1.80 (m, 2H), 1.48 (d, J=6.7 Hz, 3H), 1.35-1.28 (m, 6H), 1.05 (s, 9H); LC/MS (ESI, m/z): [(M+H)]+=1368.8.

Step 4—(2-{[(5S, 8S, 10aR)-3-acetyl-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-6-oxo-octahydropyrrolo[1, 2-a][1, 5]diazocin-5-yl]carbamoyl}-1-benzothiophen-5-yl)difluoromethylphosphonate (NH4+ ion). To a stirred solution of methyl (5S, 8S, 10aR)-8-{[(2S)-4-carbamoyl-1-[2-chloro-3-(3-{[(2S)-1-[(2S, 4R)-4-hydroxy-2-{[(1S)-1-[4-(4-methyl-1, 3-thiazol-5-yl)phenyl]ethyl]carbamoyl}pyrrolidin-1-yl]-3, 3-dimethyl-1-oxobutan-2-yl]carbamoyl}propyl)phenoxy]butan-2-yl]carbamoyl}-5-{5-[(diethoxyphosphoryl)difluoromethyl]-1-benzothiophene-2-amido}-6-oxo-octahydropyrrolo[1, 2-a][1, 5]diazocine-3-carboxylate (100 mg, 0.074 mmol) in DCM (2.5 mL) was added TMSI (73.08 mg, 0.37 mmol) and N,O-bis(trimethylsilyl)hydroxylamine (64.79 mg, 0.365 mmol) at rt under nitrogen atmosphere and the reaction mixture was stirred for 1 h at rt. On completion, the resulting mixture was concentrated under reduced pressure. The residue was purified by reverse flash chromatography (column, Welflash™ C18-1, 20-40 um, 120 g; Eluent A: Water (plus 10 mmol/L NH₄HCO₃); Eluent B: ACN; Gradient 15% to 45% B in 30 min; Flow rate: 60 mL/min; Detector: 220/254 nm; desired fractions were collected at 31% B) and concentrated under reduced pressure to afford the title compound (41.8 mg, 41% yield) as a white solid. 1H NMR (300 MHz, DMSO-d6) δ 9.09-8.94 (m, 2H), 8.38 (d, J=7.7 Hz, 1H), 8.23 (d, J=9.4 Hz, 1H), 8.05-7.96 (m, 2H), 7.90-7.86 (m, 1H), 7.63 (d, 1=8.7 Hz, 1s), 7.46-7.38 (s, 41H), 7.25-7.09 (in, 41), 7.00 (d, J=8.3 Hz, 1H), 6.90 (d, J=7.6 Hz, 1H1), 6.72 (d, =7.7 Hz, 1 m), 5.0-4.82 (n, 3H), 4.54 (d, =9.3 Hz, H1), 4.47-4.38 (m, 1H), 4.36-4.25 (i, 31), 4.07-3.78 (n, 56-6), 3.72-3.60 (n, 7H), 2.75-2.63 (in, 2 m), 2.45 (s, 3H), 2.37-2.29 (m, 21H), 2.22-2.14 (i, 41), 2.08-1.97 (s, 3H), 1.90-1.70 (m, 8H), 1.38 (d, =7.0 Hz, 3-1), 0.96 (s, 9H); LC/MS (ESI, m/z): [(M+H)]+=1312.5.

TABLE 6
Compounds synthesized via Method 4, where the corresponding amines and acids were
coupled in Step 1; and the correspond acid was coupled in Step 3.

| | | | | LC/MS | |
|---|---|---|---|---|---|
| | Step 1 | Step 1 | Step 3 | (ESI, m/z) | |
| I-#ª | Amine | Acid | Acid | (M + H)⁺ | ¹H NMR (400 MHZ, DMSO-d6) δ |
| I-82 | F | H | AY | 1274.6 | 12.06 (s, 1H), 8.98 (s, 1H), 8.90 (s, 1H), 8.80-8.76 (m, |
| | | | | | 1H), 8.43-8.39 (m, 1H), 8.37-8.33 (m, 1H), 8.22-8.17 (m, |
| | | | | | 2H), 8.03-87.98 (m, 1H), 7.89-7.82 (m, 1H), 7.46-7.42 (m, |
| | | | | | 2H), 7.40-7.36 (m, 2H), 7.25-7.14 (m, 2H), 7.02-6.98 (m, |
| | | | | | 1H), 6.91-6.87 (m, 1H), 6.73-6.69 (m, 1H), 5.08 (s, 1H), |
| | | | | | 5.06-4.96 (m, 1H), 4.94-4.90 (m, 1H), 4.56-4.52 (m, 1H), |
| | | | | | 4.44-4.40 (m, 1H), 4.39-4.31 (m, 1H), 4.31-4.21 (m, 2H), |
| | | | | | 4.10-3.99 (m, 2H), 3.97-3.80 (m, 1H), 3.72-3.57 (m, 3H), |
| | | | | | 3.40 (s, 3H), 2.73-2.64 (m, 2H), 2.46 (s, 3H), 2.35-2.17 |
| | | | | | (m, 2H), 2.19-2.15 (m, 3H), 2.18-2.14 (m, 2H), 2.16-2.12 |
| | | | | | (m, 1H), 2.15-2.11 (m, 1H), 2.03-1.98 (m, 1H), 1.92-1.87 |
| | | | | | (m, 2H), 1.84-1.65 (m, 7H), 1.38 (d, J = 7.0 Hz, 3H), 1.27- |
| | | | | | 1.22 (m, 1H), 0.95 (s, 9H) |
| I-83 | F | H | AX | 1296.3 | 8.99 (s, 1H), 8.41-8.30 (m, 2H), 8.18-8.07 (m, 3H), 8.06- |
| | | | | | 7.98 (m, 1H), 7.88 (d, J = 9.1 Hz, 1H), 7.64-7.56 (m, 1H), |
| | | | | | 7.46-7.34 (m, 4H), 7.27-7.16 (m, 2H), 7.03-6.96 (m, 1H), |
| | | | | | 6.93-6.85 (m, 1H), 6.81-6.65 (m, 1H), 5.05-4.85 (m, 2H), |
| | | | | | 4.54 (d, J = 9.3 Hz, 1H), 4.47-4.21 (m, 3H), 4.21-3.99 (m, |
| | | | | | 4H), 3.99-3.79 (m, 1H), 3.79-3.57 (m, 3H), 3.40 (t, J = |
| | | | | | 11.8 Hz, 2H), 3.10-2.96 (m, 2H), 2.69 (d, J = 8.3 Hz, 2H), |
| | | | | | 2.46 (s, 3H), 2.36-2.28 (m, 2H), 2.23-2.09 (m, 6H), 2.07- |
| | | | | | 1.96 (m, 2H), 1.95-1.64 (m, 10H), 1.38 (d, J = 7.0 Hz, |
| | | | | | 3H), 0.95 (s, 9H) |
| I-86 | F | AW | AY | 1290.3 | 9.10-8.91 (m, 3H), 8.39-8.29(m, 2H), 8.22 (d, J = 8.5 Hz, |
| | | | | | 1H), 8.08 (d, J = 8.5 Hz, 1H), 8.00 (d, J = 8.1 Hz, 1H), |
| | | | | | 7.86 (d, J = 9.2 Hz, 1H), 7.48-7.44 (m, 2H), 7.42-7.34 (m, |
| | | | | | 2H), 7.26-8.19 (m, 2H), 7.14-7.06 (m, 1H), 7.00 (d, J = |
| | | | | | 8.2 Hz, 1H), 6.89 (d, J = 7.6 Hz, 1H), 6.76-6.71 (m, 1H), |
| | | | | | 4.99-4.85 (m, 2H), 4.54 (d, J = 9.2 Hz, 1H), 4.48-4.37 (m, |
| | | | | | 1H), 4.37-4.22 (m, 2H), 4.13-3.95 (m, 2H), 4.12-3.96 (m, |
| | | | | | 1H), 3.84-3.71 (m, 1H), 3.71-3.56 (m, 6H), 3.52 (s, 3H), |
| | | | | | 3.09-3.00 (m, 2H), 2.73-2.61 (m, 2H), 2.46 (s, 3H), 2.36- |
| | | | | | 2.25 (m, 2H), 2.24-2.07 (m, 4H), 2.05-1.90 (m, 2H), 1.88- |
| | | | | | 1.65 (m, 8H), 1.38 (d, J = 6.9 Hz, 3H), 0.95 (s, 9H) |
| I-87 | F | AW | B | 1273.5 | 11.86 (d, J = 10.6 Hz, 1H), 9.00 (s, 1H), 8.89 (s, 1H), 8.80- |
| | | | | | 8.67 (m, 1H), 8.39 (d, J = 7.8 Hz, 1H), 8.07-7.95 (m, 2H), |
| | | | | | 7.89 (d, J = 9.2 Hz, 1H), 7.48-7.36 (m, 7H), 7.25-7.14 (m, |
| | | | | | 2H), 7.01 (d, J = 8.3 Hz, 1H), 6.90 (d, J = 7.6 Hz, 1H), |
| | | | | | 6.80-6.71 (m, 1H), 5.04-4.87 (m, 2H), 4.55 (d, J = 9.3 Hz, |
| | | | | | 1H), 4.49-4.42 (m, 1H), 4.40-4.20 (m, 3H), 4.10-3.98 (m, |
| | | | | | 2H), 3.91-3.79 (m, 3H), 3.71-3.57 (m, 6H), 3.56-3.51 (m, |
| | | | | | 3H), 2.79-2.61 (m, 2H), 2.47 (s, 3H), 2.36-2.26 (m, 2H), |
| | | | | | 2.26-1.96 (m, 4H), 1.95-1.68 (m, 10H), 1.39 (d, J = 7.0 |
| | | | | | Hz, 3H), 0.96 (s, 9H) |
| I-88 | F | A | AY | 1232.5 | 9.02-8.90 (m, 3H), 8.42-8.31 (m, 2H), 8.24-8.18 (m, 1H), |
| | | | | | 8.10-7.99 (m, 2H), 7.87 (d, J = 9.3 Hz, 1H), 7.48-7.34 (m, |
| | | | | | 4H), 7.26-7.15 (m, 2H), 7.03-6.96 (m, 1H), 6.89 (d, J = |
| | | | | | 7.6 Hz, 1H), 6.75 (s, 1H), 5.03-4.84 (m, 2H), 4.58-4.38 |
| | | | | | (m, 2H), 4.35-4.26 (m, 2H), 4.16-3.87 (m, 3H), 3.64-3.59 |
| | | | | | (m, 2H), 3.06-3.02 (m, 2H), 2.69-2.65 (m, 1H), 2.46 (s, |
| | | | | | 3H), 2.37-2.25 (m, 1H), 2.25-2.15 (m, 3H), 2.12-1.95 (m, |
| | | | | | 3H), 1.95-1.73 (m, 10H), 1.74-1.63 (m, 2H), 1.62-1.46 (m, |
| | | | | | 2H), 1.41-1.35 (m, 3H), 1.22-1.12 (m, 4H), 0.95 (s, 9H) |
| I-89 | F | A | AX | 1253.5 | 8.99 (s, 1H), 8.91 (d, J = 6.8 Hz, 1H), 8.38 (d, J = 7.7 Hz, |
| | | | | | 1H), 8.29 (s, 1H), 8.04-7.98 (m, 2H), 7.88 (d, J = 9.2 Hz, |
| | | | | | 1H), 7.63 (d, J = 8.7 Hz, 1H), 7.47-7.34 (m, 4H), 7.33- |
| | | | | | 7.15 (m, 3H), 7.03-6.97 (m, 1H), 6.92-6.86 (m, 1H), 6.75 |
| | | | | | (s, 1H), 5.0-4.84 (m, 2H), 4.54 (d, J = 9.3 Hz, 1H), 4.47- |
| | | | | | 4.38 (m, 1H), 4.35-4.25 (m, 4H), 4.15-3.98 (m, 2H), 3.95- |
| | | | | | 3.89 (m, 1H), 3.65-3.58 (m, 2H), 2.76-5.64 (m, 2H), 2.46 |
| | | | | | (s, 3H), 2.34-2.14 (m, 4H), 2.13-1.73 (m, 4H), 1.93-1.73 |
| | | | | | (m, 10H), 1.72-1.65 (m, 4H), 1.61-1.44 (m, 2H), 1.38 (d, |
| | | | | | J = 7.0 Hz, 3H), 0.96 (s, 9H) |
| I-97^{b} | BL | BK | AX | 1172.7 | 11.09 (s, 1H), 8.58-8.54 (m 2H), 8.39-8.35 (m, 1H), 8.31- |
| | | | | | 8.27 (m, 1H), 8.13 (s, 2H), 7.66-7.62 (m, 1H), 7.27-7.18 |
| | | | | | (m, 4H), 7.06 (s, 1H), 7.03-6.98 (m, 1H), 6.90-6.86 (m, |
| | | | | | 3H), 6.74 (s, 1H), 5.34 (dd, J = 12.8, 5.4 Hz, 1H), 4.95 (s, |
| | | | | | 1H), 4.46-4.42 (m, 1H), 4.19-4.15 (m, 3H), 3.77-3.73 (m, |
| | | | | | 2H), 3.34 (s, 3H), 2.91-2.87 (m, 1H), 2.65-2.59 (m, 8H), |
| | | | | | 2.14-2.07 (m, 4H), 2.04-2.00 (m, 3H), 1.92-1.88 (m, 1H), |
| | | | | | 1.66-1.47 (m, 15H), 0.98-0.92 (m, 6H) |
| I-98 | F | AZ | BA | 1312.5 | 8.99 (s, 1H), 8.78-8.76 (m, 1H), 8.41-8.39 (m, 1H), 8.32 |
| | | | | | (s, 1H), 8.12-8.01 (m, 2H), 7.92-7.88 (m, 1H), 7.66-7.62 |
| | | | | | (m, 1H), 7.46-7.37 (m, 3H), 7.29-7.16 (m, 2H), 7.02-6.99 |
| | | | | | (m, 1H), 6.91-6.89 (m, 1H), 6.78 (s, 1H), 6.51 (s, 1H), |
| | | | | | 4.95-4.92 (m, 1H), 4.79-4.75 (m, 1H), 4.56-4.52 (m, 1H), |
| | | | | | 4.46-4.42 (m, 2H), 4.31-4.28 (m, 1H), 4.17-4.11-3.92 (m, |
| | | | | | 5H), 3.78-3.74 (m, 2H), 3.64-3.60 (m, 2H), 3.30-3.27 (m, |
| | | | | | 1H), 3.20-3.14 (m, 3H), 2.71-2.64 (m, 4H), 2.48-2.44 (m, |
| | | | | | 2H), 2.22-2.18 (m, 3H), 2.02-1.97 (m, 1H), 1.85-1.67 (m, |
| | | | | | 12H), 1.60-1.56 (m, 1H), 1.42-1.20 (m, 6H), 0.95 (s, 9H) |

ªStep 1 was run anywhere from 3-12 h at rt. Step 2 was run anywhere from 1-4 h at rt. Other deprotection agents, like TFA, can also be employed. Step 3 was run anywhere from 1-2 h at rt. TEA in NMP could also be used for the coupling in place of HATU.
^{b}No Step 2 deprotection was required.

### Example 8 (Method 5): Synthesis of 2-[[(5S,8S,10aR)-8-[[(2S)-4-Carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy] butan-2-yl] carbamoyl]-6-oxo-3-[2-[(1s,4s)-4-[[1-(2,6-dioxopiperidin-3-yl)-3-methyl-2-oxo-1,3-benzodiazol-5-yl]methyl] cyclohexyl]acetyl]-octahydropyrrolo [1,2-a][1,5] diazocin-5-yl] carbamoyl]-1H-indole-5-carbonylphosphonic acid (I-36)

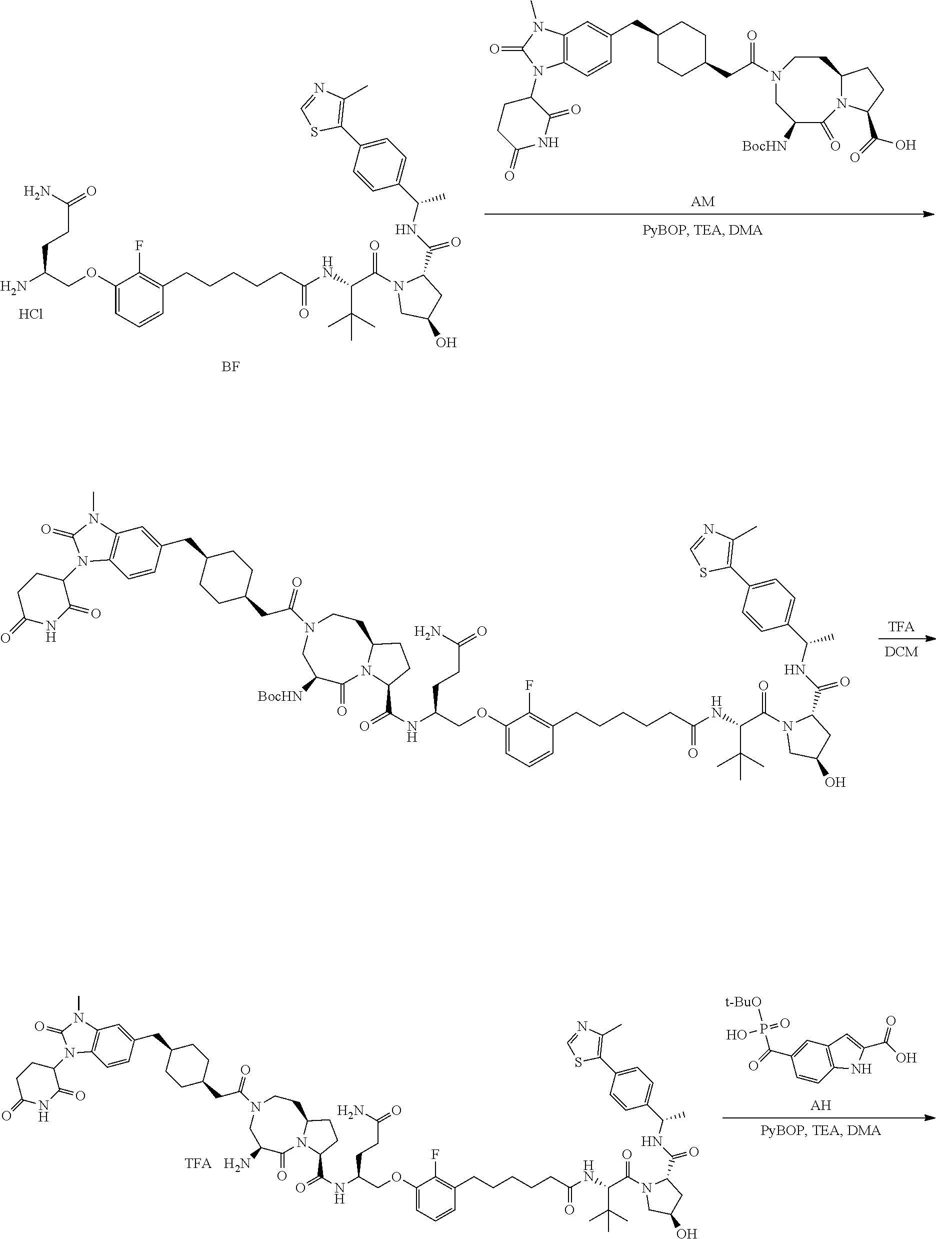

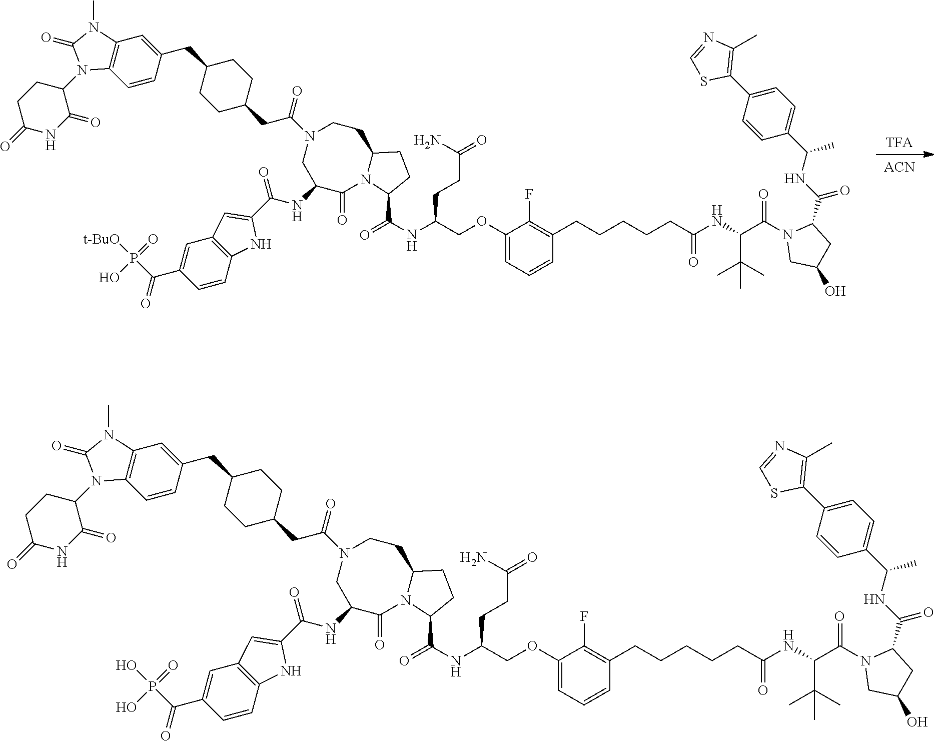

Step 1—Tert-butyl N-[(5S,8S,10aR)-8-[[(2S)-4-carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy] butan-2-yl] carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo[1,2-a][1,5]diazocin-5-yl] carbamate. To a stirred mixture of (2S,4R)-1-[(2S)-2-(6-[3-[(2S)-2-amino-4-carbamoylbutoxy]-2-fluorophenyl] hexanamido)-3,3-dimethylbutanoyl]-4-hydroxy-N-[(1S)-1-[4-(4-methyl-1,3-thiazol-5-yl) phenyl] ethyl] pyrrolidine-2-carboxamide hydrochloride (346.10 mg, 0.431 mmol, Intermediate BF) and TEA (130.77 mg, 1.29 mmol) in DMA (5.00 mL) were added (5S,8S,10aR)-5-[(tert-butoxycarbonyl) amino]-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5] diazocine-8-carboxylic acid (311.38 mg, 0.431 mmol, Intermediate AM) and PyBOP (269.01 mg, 0.517 mmol) at 0° C. under nitrogen atmosphere. Then the resulting mixture was stirred for 1 h at 25° C. under nitrogen atmosphere. On completion, the resulting mixture was concentrated under reduced pressure. The residue was purified by reverse phase flash chromatography (Column: WelFlash™ C18-I, 20-40 m, 330 g; Eluent A: Water (plus 10 mmol/L FA); Eluent B: ACN; Gradient: 50%-70% B in 30 min; Flow rate: 80 mL/min; Detector: 220/254 nm; desired fractions were collected at 60% B) and concentrated under reduced pressure to afford the title compound (446 mg, 70% yield) as a white solid. ¹H NMR (300 MHz, Chloroform-d) δ 8.93 (s, 1H), 7.62-7.54 (m, 1H), 7.46-7.38 (m, 4H), 7.32-7.26 (m, 5H), 7.03-6.96 (m, 1H), 6.88-6.54 (m, 6H), 5.93-5.52 (m, 2H), 5.31-5.27 (m, 1H), 5.16-4.99 (m, 1H), 4.78-4.69 (m, 1H), 4.66-4.41 (m, 2H), 4.39-4.31 (m, 1H), 4.19-4.05 (m, 5H), 3.71-3.64 (m, 1H), 3.43 (s, 3H), 3.34-3.06 (m, 1H), 3.01-2.71 (m, 2H), 2.69-2.62 (m, 1H), 2.59 (s, 3H), 2.52-2.39 (m, 1H), 2.32-2.11 (m, 14H), 1.92-1.70 (m, 2H), 1.69-1.48 (m, 30H), 1.46 (d, J=6.9 Hz, 3H), 1.36-1.28 (m, 1H), 1.21-1.10 (m, 1H), 1.04 (s, 9H); LC/MS (ESI, m/z): [(M+H)]+=1471.6.

Step 2—(2S,4R)-1-[(2S)-2-(6-[3-[(2S)-2-[[(5S,8S,10aR)-5-Amino-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5] diazocin-8-yl] formamido]-4-carbamoylbutoxy]-2-fluorophenyl]hexanamido)-3,3-dimethylbutanoyl]-4-hydroxy-N-[(1S)-1-[4-(4-methyl-1,3-thiazol-5-yl) phenyl] ethyl]pyrrolidine-2-carboxamide trifluoroacetate. To a stirred solution of tert-butyl N-[(5S,8S,10aR)-8-[[(2S)-4-carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy]butan-2-yl] carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5] diazocin-5-yl] carbamate (446.90 mg, 0.304 mmol) in DCM (8 mL) was added TFA (1.50 mL, 20.20 mmol) at 0° C. under nitrogen atmosphere. Then the resulting mixture was stirred for 2 h at 25° C. under nitrogen atmosphere. On completion, the reaction mixture was concentrated under reduced pressure to afford the title compound (440 mg, 99% yield) as a white solid. LC/MS (ESI, m/z): [(M+H)]+=1371.6.

Step 3—2-[[(5S,8S,10aR)-8-[[(2S)-4-Carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy] butan-2-yl] carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5]diazocin-5-yl] carbamoyl]-1H-indole-5-carbonyl (tert-butoxy) phosphinic acid. To a stirred mixture of (2S,4R)-1-[(2S)-2-(6-[3-[(2S)-2-[[(5S,8S,10aR)-5-amino-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5] diazocin-8-yl] formamido]-4-carbamoylbutoxy]-2-fluorophenyl]hexanamido)-3,3-dimethylbutanoyl]-4-hydroxy-N-[(1S)-1-[4-(4-methyl-1,3-thiazol-5-yl) phenyl] ethyl]pyrrolidine-2-carboxamide trifluoroacetate (303.40 mg, 0.206 mmol) and TEA (0.06 mL, 0.618 mmol) in DMA (5 mL) were added 5-[[tert-butoxy (hydroxy)phosphoryl]carbonyl]-1H-indole-2-carboxylic acid (67.14 mg, 0.206 mmol, Intermediate AH) and PyBOP (128.91 mg, 0.248 mmol) at 0° C. under nitrogen atmosphere. The resulting mixture was then stirred for 1 h at 25° C. under nitrogen atmosphere. On completion, the reaction mixture was concentrated under reduced pressure. The residue was purified by reverse phase flash chromatography (Column: WelFlash™ C18-I, 20-40 m, 120 g; EluentA: Water (plus 10 mmol/L FA); Eluent B: ACN; Gradient: 30%-60% B in 30 min; Flow rate: 50 mL/min; Detector: 220/254 nm; desired fractions were collected at 50% B) and concentrated under reduced pressure to afford the title compound (160 mg, 46% yield) as a white solid. ¹H NMR (400 MHz, DMSO-d6) δ 12.16-11.97 (m, 1H), 11.08 (s, 1H), 8.99 (s, 1H), 8.83 (s, 1H), 8.53 (s, 1H), 8.37 (d, J=7.8 Hz, 1H), 8.11-7.94 (m, 2H), 7.79 (d, J=9.2 Hz, 1H), 7.56-7.49 (m, 1H), 7.48-7.33 (m, 6H), 7.28-7.21 (m, 1H), 7.05-6.95 (m, 4H), 6.86-6.64 (m, 3H), 5.33 (dd, J=12.7, 5.2 Hz, 1H), 5.18-4.87 (m, 3H), 4.51 (d, J=9.3 Hz, 1H), 4.45-4.34 (m, 2H), 4.32-4.14 (m, 1H), 4.09-3.86 (m, 4H), 3.84-3.68 (m, 1H), 3.64-3.57 (m, 3H), 3.42-3.35 (m, 2H), 3.28 (s, 3H), 3.05-2.82 (m, 2H), 2.76-2.53 (m, 8H), 2.48-2.44 (m, 3H), 2.36-2.05 (m, 6H), 2.07-1.95 (m, 4H), 1.91-1.62 (m, 7H), 1.61-1.40 (m, 13H), 1.39 (s, 9H), 1.387-1.20 (m, 4H), 0.93 (s, 9H); LC/MS (ESI, m/z): [(M+H)]+=1679.9.

Step 4—2-[[(5S,8S,10aR)-8-[[(2S)-4-Carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy] butan-2-yl] carbamoyl]-6-oxo-3-[2-[(1s,4s)-4-[[1-(2,6-dioxopiperidin-3-yl)-3-methyl-2-oxo-1,3-benzodiazol-5-yl]methyl] cyclohexyl] acetyl]-octahydropyrrolo [1,2-a][1,5] diazocin-5-yl] carbamoyl]-1H-indole-5-carbonylphosphonic acid. To a stirred solution of 2-[[(5S,8S,10aR)-8-[[(2S)-4-carbamoyl-1-[2-fluoro-3-(5-[[(2S)-1-[(2S,4R)-4-hydroxy-2-[[(1S)-1-[4-(4-methyl-1,3-thiazol-5-yl) phenyl] ethyl] carbamoyl] pyrrolidin-1-yl]-3,3-dimethyl-1-oxobutan-2-yl] carbamoyl] pentyl) phenoxy] butan-2-yl] carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] acetyl]-octahydropyrrolo [1,2-a][1,5] diazocin-5-yl] carbamoyl]-1H-indole-5-carbonyl (tert-butoxy) phosphinic acid (145.70 mg, 0.087 mmol) in ACN (6.00 mL) was added TFA (1.00 mL, 13.46 mmol) at 25° C. under nitrogen atmosphere. The resulting mixture was stirred for 2 h at 25° C. under nitrogen atmosphere. On completion, the reaction mixture was concentrated under reduced pressure. The residue was purified by reverse phase flash chromatography (Column: WelFlash™ C18-I, 20-40 m, 120 g; Eluent A: Water (plus 10 mmol/L FA); Eluent B: ACN; Gradient: 25%-55% B in 30 min; Flow rate: 50 mL/min; Detector: 220/254 nm; desired fractions were collected at 43% B) and concentrated under reduced pressure to the title compound (32.8 mg, 23% yield) as a white solid. ¹H NMR (400 MHz, DMSO-d6) δ 12.16-11.97 (m, 1H), 11.08 (s, 1H), 8.99 (s, 1H), 8.83 (s, 1H), 8.53 (s, 1H), 8.37 (d, J=7.8 Hz, 1H), 8.11-7.94 (m, 2H), 7.79 (d, J=9.2 Hz, 1H), 7.56-7.49 (m, 1H), 7.48-7.33 (m, 6H), 7.28-7.21 (m, 1H), 7.05-6.95 (m, 4H), 6.86-6.64 (m, 3H), 5.33 (dd, J=12.7, 5.2 Hz, 1H), 5.18-4.87 (m, 3H), 4.51 (d, J=9.3 Hz, 1H), 4.45-4.34 (m, 2H), 4.32-4.14 (m, 2H), 4.09-3.86 (m, 4H), 3.84-3.68 (m, 1H), 3.64-3.57 (m, 3H), 3.42-3.35 (m, 2H), 3.28 (s, 3H), 3.05-2.82 (m, 2H), 2.76-2.53 (m, 8H), 2.48-2.44 (m, 3H), 2.36-2.05 (m, 6H), 2.07-1.95 (m, 4H), 1.91-1.62 (m, 7H), 1.61-1.19 (in, 171H), 0.93 (s, 9H); LC/MS (ESI, m/z): [(M+H)]+=1622.7.

TABLE 7
Compounds synthesized via Method 5, where the corresponding amines and acids were
coupled in Step 1.

| | | | LC/MS | |
|---|---|---|---|---|
| | Step 1 | Step 1 | (ESI, m/z) | |
| I-#ª | Amine | Acid | (M + H)⁺ | ¹H NMR (400 MHZ, DMSO-d6) δ |
| I-3 | BN | A | 1212.5 | 11.81 (s, 1H), 8.99 (s, 1H), 8.89 (s, 1H), 8.67-8.63 (m, 1H), 8.41- |
| | | | | 8.37 (m, 1H), 8.05-8.01 (m, 1H), 7.99-7.90 (m, 1H), 7.84-7.80 (m, |
| | | | | 1H), 7.42-7.35 (m, 6H), 7.18-7.10 (m, 2H), 7.02-6.98 (m, 2H), 6.88- |
| | | | | 6.75 (m, 1H), 5.08-5.02 (m, 1H), 4.93-4.89 (m, 1H), 4.54-4.50 (m, |
| | | | | 1H), 4.44-4.39 (m, 1H), 4.31-4.20 (m, 3H), 4.07-3.90 (m, 3H), 3.63- |
| | | | | 3.59 (m, 2H), 3.52-3.35 (m, 3H), 2.59-2.55 (m, 2H), 2.47 (s, 3H), |
| | | | | 2.39-2.25 (m, 1H), 2.24-2.10 (m, 3H), 2.09-1.95 (m, 3H), 1.94-1.78 |
| | | | | (m, 7H), 1.76-1.45 (m, 10H), 1.41-1.37 (m, 3H), 0.94 (s, 9H) |
| I-4 | CF | A | 1240.5 | 11.81 (s, 1H), 8.99 (s, 1H), 8.89 (s, 1H), 8.67-8.62 (m, 1H), 8.41- |
| | | | | 8.37 (m, 1H), 8.05-8.01 (m, 1H), 7.99-7.90 (m, 1H), 7.84-7.80 (m, |
| | | | | 1H), 7.42-7.35 (m, 6H), 7.18-7.10 (m, 2H), 7.02-6.98 (m, 2H), 6.88- |
| | | | | 6.75 (m, 1H), 5.08-5.02 (m, 1H), 4.93-4.89 (m, 1H), 4.54-4.50 (m, |
| | | | | 1H), 4.44-4.39 (m, 1H), 4.31-4.20 (m, 3H), 4.07-3.90 (m, 3H), 3.63- |
| | | | | 3.59 (m, 2H), 3.52-3.35 (m, 3H), 2.59-2.55 (m, 2H), 2.47 (s, 3H), |
| | | | | 2.22-2.12 (m, 7H), 2.09-1.95 (m, 3H), 1.94-1.78 (m, 8H), 1.76-1.45 |
| | | | | (m, 10H), 1.41-1.37 (m, 3H), 1.29-1.26 (m, 2H), 0.94 (s, 9H) |
| I-9 | DA | A | 1198.5 | 11.81 (s, 1H), 8.99 (s, 1H), 8.89 (s, 1H), 8.66 (d, J = 6.9 Hz, 1H), |
| | | | | 8.40 (d, J = 7.8 Hz, 1H), 8.03 (d, J = 8.2 Hz, 1H), 7.96-6.87 (m, |
| | | | | 2H), 7.49-7.34 (m, 6H), 7.19 (s, 1H), 7.09-6.98 (m, 2H), 6.85-6.73 |
| | | | | (m, 2H), 5.03-5.01 (m, 2H), 4.98-4.87 (m, 1H), 4.56-4.52 (m, 1H), |
| | | | | 4.47-4.40 (m, 1H), 4.41-4.32 (m, 3H), 4.10-3.89 (m, 3H), 3.65-3.58 |
| | | | | (m, 2H), 2.50-2.47 (m, 2H), 2.46 (s, 3H), 2.25-2.15 (m, 3H), 2.12- |
| | | | | 2.00 (m, 3H), 1.98-1.45 (m, 17H), 1.38 (d, J = 6.9 Hz, 3H), 0.95 (s, |
| | | | | 9H) |
| I-26 | DX | AM | 986.6 | 12.22-11.51(m, 1H), 8.95-8.90 (m, 1H), 8.55-8.43 (m, 1H), 8.00- |
| | | | | 7.92 (m, 1H), 7.70-7.61 (m, 1H), 7.47-7.40 (m, 1H), 7.39-7.33 (m, |
| | | | | 1H), 7.29-7.21 (m, 1H), 7.05-6.92 (m, 3H), 6.84-6.65 (m, 2H), 5.41- |
| | | | | 5.28 (m, 1H), 5.10-4.96 (m, 1H), 4.35-4.16 (m, 2H), 4.04-3.66 (m, |
| | | | | 4H), 3.64-3.62 (m, 6H), 3.01-2.85(m, 2H), 2.67-2.58 (m, 3H), 2.19- |
| | | | | 1.83 (m, 10H), 1.80-1.65 (m, 4H), 1.56-1.25 (m, 12H), 0.94-0.79 |
| | | | | (m, 2H) |
| I-27 | DO | A | 1001.5 | 12.03 (s, 1H), 11.07 (s, 1H), 8.83-8.73 (m, 1H), 8.70-8.64 (m, 1H), |
| | | | | 8.08-7.87 (m, 2H), 7.58-7.37 (m, 2H), 7.27-7.11 (m, 2H), 7.06-6.86 |
| | | | | (m, 5H), 6.73 (s, 1H), 5.40-4.93 (m, 2H), 4.38-4.23 (m, 3H), 4.12- |
| | | | | 3.98 (m, 2H), 4.12-3.98 (m, 1H), 3.32 (s, 3H), 2.98-2.82 (m, 2H), |
| | | | | 2.77-2.57 (m, 6H), 2.25-2.15 (m, 2H), 2.11-1.95 (m, 3H), 1.94-1.74 |
| | | | | (m, 7H), 1.73-1.45 (m, 5H), 1.30-1.21 (m, 2H) |
| I-29 | CD | AM | 1012.5 | 12.09-12.05 (m, 1H), 11.08 (s, 1H), 8.91-8.78 (m, 1H), 8.61-8.57 |
| | | | | (m, 1H), 7.97-7.95 (m, 1H), 7.60-7.43 (m, 3H), 7.26-7.19 (m, 1H), |
| | | | | 7.05-6.94 (m, 2H), 6.87-6.79 (m, 1H), 6.68-6.65 (m, 1H), 5.36-5.30 |
| | | | | (m, 1H), 5.02-4.95 (m, 1H), 4.35-4.30 (m, 1H), 4.22-4.19 (m, 2H), |
| | | | | 4.10-3.81 (m, 4H), 3.50-3.35 (m, 3H), 3.32 (s, 3H), 2.91-2.86 (m, |
| | | | | 1H), 2.65-2.59 (m, 4H), 2.19-1.86 (m, 12H), 1.80-1.54 (m, 2H), |
| | | | | 1.53-1.32 (m, 15H) |
| I-31 | DB | AM | 984.5 | 12.06-11.76 (m, 1H), 8.91-8.75 (m, 1H), 8.54 (d, J = 7.0 Hz, 1H), |
| | | | | 8.21-8.09 (m, 1H), 8.02-7.91 (m, 1H), 7.47-7.26 (m, 4H), 7.03-6.94 |
| | | | | (m, 2H), 6.88-6.75 (m, 1H), 6.74-6.66 (m, 1H), 5.38-5.30 (m, 1H), |
| | | | | 5.05-4.84 (m, 1H), 4.29-4.21 (m, 1H), 4.20-4.12 (m, 1H), 3.95-3.85 |
| | | | | (m, 1H), 3.79-3.71 (m, 1H), 3.68-3.47 (m, 3H), 3.46-3.30 (m, 5H), |
| | | | | 2.96-2.84 (m, 1H), 2.75-2.67 (m, 1H), 2.66-2.53 (m, 4H), 2.20-2.07 |
| | | | | (m, 3H), 2.06-1.90 (m, 4H), 1.85-1.62 (m, 6H), 1.56-1.28 (m, 8H), |
| | | | | 0.64-0.48 (m, 4H) |
| I-38 | DB | AM | 1195.6 | 12.26 (s, 1H), 11.08 (s, 1H), 8.91-8.73 (m, 2H), 8.51-8.47 (m, 1H), |
| | | | | 8.27-8.23 (m, 1H), 7.98-7.94 (m, 1H), 7.79-7.75 (m, 2H), 7.54-7.50 |
| | | | | (m, 1H), 7.49-7.44 (m, 1H), 7.39-7.35 (m, 2H), 7.31-7.27 (m, 1H), |
| | | | | 7.07-6.94 (m, 2H), 6.90-6.66 (m, 2H), 5.42-5.26 (m, 1H), 4.97-4.93 |
| | | | | (m, 1H), 4.50-4.46 (m, 1H), 4.20-4.16 (m, 2H), 3.99-3.95 (m, 1H), |
| | | | | 3.87-3.83 (m, 1H), 3.64-3.60 (m, 2H), 3.32 (s, 3H), 3.23-3.18 (s, |
| | | | | 3H), 2.92-2.88 (m, 1H), 2.76-2.54 (m, 7H), 2.17-2.13 (m, 3H), 2.03- |
| | | | | 1.99 (m, 5H), 1.87-1.83 (m, 3H), 1.74-1.70 (m, 3H), 1.50-1.32 (m, |
| | | | | 9H), 1.28-1.24 (m, 3H) |
| I-40 | DM | AM | 1183.4 | 12.08 (s, 1H), 11.07 (s, 1H), 8.85-8.78 (m, 1H), 8.56-8.44 (m, 2H), |
| | | | | 8.20-8.07 (m, 1H), 8.00-7.93 (m, 1H), 7.89-7.85 (m, 2H), 7.58-7.50 |
| | | | | (m, 3H), 7.46-7.43 (m, 1H), 7.27-7.20 (m, 1H), 7.02-6.94 (m, 2H), |
| | | | | 6.87-6.75 (m, 2H), 5.38-5.28 (m, 1H), 5.03-4.90 (m, 2H), 4.52-4.41 |
| | | | | (m, 1H), 4.33-4.14 (m, 2H), 4.06-3.70 (m, 3H), 3.67-3.49 (m, 2H), |
| | | | | 3.33-3.32 (m, 1H), 3.28 (s, 3H), 3.19 (s, 3H), 2.99-2.82 (m, 2H), |
| | | | | 2.76-2.55 (m, 5H), 2.44-2.08 (m, 4H), 2.05-1.95 (m, 4H), 1.94-1.61 |
| | | | | (m, 8H), 1.54-1.41 (m, 5H), 1.38 (d, J = 7.0 Hz, 3H) |
| I-41 | CW | AM | 1183.4 | 12.09 (s, 1H), 11.08 (s, 1H), 8.83-8.78 (m, 1H), 8.54-8.47 (m, 2H), |
| | | | | 8.25-8.13 (m, 1H), 7.99-7.93 (m, 1H), 7.88-7.83 (m, 2H), 7.61-7.50 |
| | | | | (m, 3H), 7.45-7.42 (m, 1H), 7.32-7.25 (m, 1H), 7.02-6.94 (m, 2H), |
| | | | | 6.87-6.72 (m, 2H), 5.39-5.27 (m, 1H), 5.04-4.82 (m, 2H), 4.48-4.39 |
| | | | | (m, 1H), 4.32-4.14 (m, 2H), 4.08-3.74 (m, 2H), 3.68-3.43 (m, 2H), |
| | | | | 3.28 (s, 3H), 3.19 (s, 3H), 2.99-2.78 (m, 2H), 2.77-2.63 (m, 2H), |
| | | | | 2.62-2.54 (m, 3H), 2.47-2.19 (m, 2H), 2.18-1.91 (m, 8H), 1.90-1.60 |
| | | | | (m, 7H), 1.54-1.42 (m, 6H), 1.41-1.37 (m, 3H) |
| I-44 | CV | AM | 1187.4 | 12.12 (s, 1H), 11.09 (s, 1H), 8.90-8.81 (m, 1H), 8.57-8.53 (m, 2H), |
| | | | | 8.39-8.23 (m, 1H), 7.99-7.95 (m, 1H), 7.77-7.73 (m, 2H), 7.62-7.58 |
| | | | | (m, 2H), 7.55-7.51 (m, 1H), 7.46-7.42 (m, 1H), 7.29-7.25 (m, 1H), |
| | | | | 6.99-6.95 (m, 2H), 6.83-6.79 (m, 2H), 5.38-5.34 (m, 1H), 5.02- |
| | | | | 54.98 (m, 1H), 4.50-4.39 (m, 3H), 4.24-4.20 (m, 3H), 3.98-3.95 (m, |
| | | | | 1H), 3.88-3.84 (m, 2H), 3.29-3.25 (m, 5H), 2.78-2.47 (m, 6H), 2.18- |
| | | | | 2.14 (m, 3H), 2.08-1.66 (m, 10H), 1.49-1.39 (m, 11H) |
| I-45 | DL | AM | 1203.4 | 12.16 (s, 1H), 11.09 (s, 1H), 8.85-8.81 (m, 2H), 8.59-8.51 (m, 2H), |
| | | | | 8.37-8.33 (m, 2H), 7.99-7.95 (m, 3H), 7.90-7.81 (m, 1H), 7.59-7.55 |
| | | | | (m, 3H), 7.46 (s, 1H), 7.30-7.26 (m, 1H), 7.04-6.94 (m, 3H), 6.84- |
| | | | | 6.80 (m, 2H), 5.35-5.31 (m, 1H), 5.03-5.00 (m, 1H), 4.47-4.43 (m, |
| | | | | 4H), 4.35-4.14 (m, 3H), 4.02-3.77 (m, 1H), 3.33-3.29 (m, 6H), 2.92- |
| | | | | 2.88 (m, 1H), 2.53-2.46 (m, 4H), 2.19-2.15 (m, 3H), 2.05-1.97 (m, |
| | | | | 5H), 1.77-1.70 (m, 4H), 1.47-1.43 (m, 9H) |
| I-46 | CU | AM | 1187.4 | 12.14 (s, 1H), 11.09 (s, 1H), 8.86-8.82 (m, 1H), 8.61-8.57 (m, 1H), |
| | | | | 8.50-8.46 (m, 1H), 8.37-8.33 (m, 1H), 7.99-7.95 (m, 1H), 7.82-7.78 |
| | | | | (m, 1H), 7.55-7.51 (m, 1H), 7.46-7.42 (m, 1H), 7.40-7.24 (m, 3H), |
| | | | | 7.04-6.93 (m, 2H), 6.89-6.72 (m, 2H), 5.35-5.31 (m, 1H), 5.01-4.97 |
| | | | | (m, 1H), 4.45-4.41 (m, 3H), 4.24 4.20 (m, 2H), 4.04-3.78 (m, 2H), |
| | | | | 3.33-3.30 (m, 6H), 2.95-2.91 (m, 1H), 2.64-2.60 (m, 6H), 2.17-2.13 |
| | | | | (m, 2H), 2.05-1.98 (m, 5H), 1.82-1.70 (m, 10H), 1.49-1.41 (m, 9H) |
| I-47 | BO | DJ | 1170.4 | 12.12-11.95 (m, 1H), 11.04 (s, 1H), 8.82 (s, 1H), 8.62-8.54 (m, 1H), |
| | | | | 8.46 (d, J = 6.8 Hz, 1H), 8.35-8.25 (m, 2H), 8.01-7.93 (m, 1H), 7.90- |
| | | | | 7.83 (m, 2H), 7.52-7.49 (m, 3H), 7.43 (s, 1H), 7.30 (s, 1H), 6.85- |
| | | | | 6.78 (m, 2H), 6.40 (s, 1H), 6.30 (d, J = 8.5 Hz, 1H), 5.23 (dd, J = |
| | | | | 12.5, 5.4 Hz, 1H), 5.01 (m, 1H), 4.43-4.35 (m, 3H), 4.25-4.20 (m, |
| | | | | 3H), 3.95-3.70(m, 4H), 3.26 (s, 3H), 3.19 (s, 3H), 2.89-2.80 (m, |
| | | | | 2H), 2.73-2.63 (m, 2H), 2.15-2.09(m, 4H), 1.97-1.80 (m, 11H), |
| | | | | 1.75-1.38 (m, 6H), 1.15-1.01 (m, 2H) |
| I-48 | BO | DH | 1170.4 | 12.13-11.95 (m, 1H), 11.04 (s, 1H), 8.82 (s, 1H), 8.58-8.51 (m, 1H), |
| | | | | 8.43 (s, 1H), 8.31 (d, J = 7.7 Hz, 1H), 7.97 (d, J = 9.0 Hz, 1H), 7.92- |
| | | | | 7.84 (m, 2H), 7.54-7.47 (m, 3H), 7.43 (d, J = 9.4 Hz, 1H), 7.28 (s, |
| | | | | 1H), 6.85-6.78 (m 2H), 6.42 (s, 1H), 6.34-6.28 (m, 2H), 5.23 (dd, |
| | | | | J = 12.8, 5.3 Hz, 1H), 5.04-4.98 (m, 1H), 4.46-4.40 (m, 2H), 4.28- |
| | | | | 4.20 (m, 2H), 4.01-3.93 (m, 1H), 3.76-3.52 (m, 5H), 3.26 (s, 3H), |
| | | | | 3.19 (s, 3H), 3.05-2.98 (m, 3H), 2.89-2.80 (m, 2H), 2.63-2.50 (m, |
| | | | | 2H), 2.20-2.08 (m, 3H), 2.01-1.90 (m, 5H), 1.78-1.60 (m, 12H) |

ªIntermediate AH was used for the coupling in Step 3 unless otherwise stated. HATU, TEA in DMA at rt could also be employed for the coupling in Step 1. HCl in DCM or dioxane at rt could also be used for the deprotection in Step 2. For Step 4, DCM could also be used as solvent.

### Example 9 (Method 6): Synthesis of (2-(((3S,6S)-6-(((S)-5-amino-1l-((4-(methylsulfonyl)benzyl)amino)-1,5-dioxopentan-2-yl)carbamoyl)-9-(6-(1-(2,6-dioxopiperidin-3-yl)-3-methyl-2-oxo-2,3-dihydro-1H-benzo[d]imidazol-5-yl)hexyl)-4-oxo-1,2,3,4,6,7-hexahydroazepino[3,2,1-hi]indol-3-yl)carbamoyl)-1H-indole-5-carbonyl)phosphonic acid (1-81)

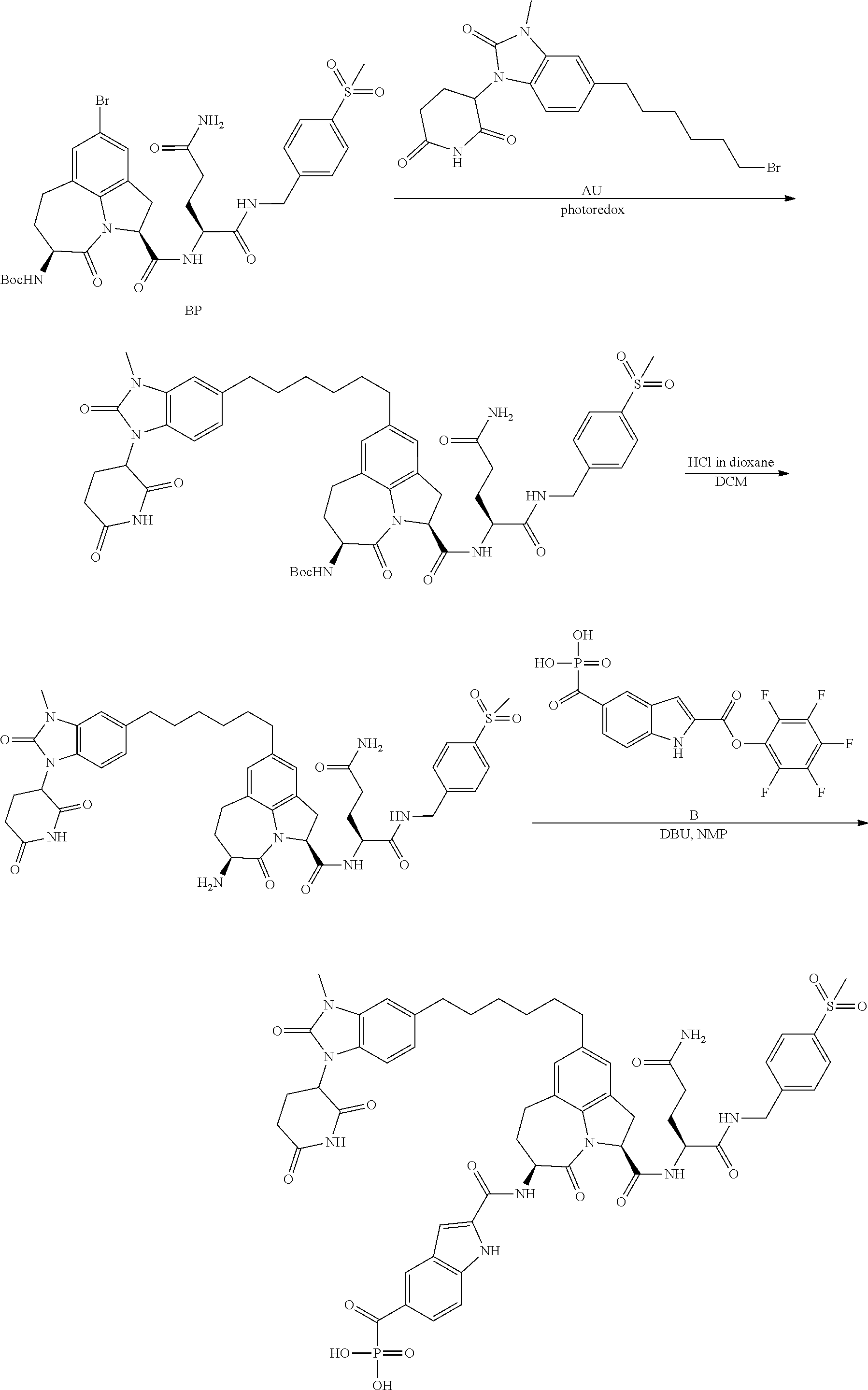

Step 1—Tert-butyl ((3S,6S)-6-(((S)-5-amino-1-((4-(methylsulfonyl)benzyl)amino)-1,5-dioxopentan-2-yl)carbamoyl)-9-(6-(1-(2,6-dioxopiperidin-3-yl)-3-methyl-2-oxo-2,3-dihydro-1H-benzo[d]imidazol-5-yl)hexyl)-4-oxo-1,2,3,4,6,7-hexahydroazepino[3,2,1-hi]indol-3-yl)carbamate. To an 8 mL vial equipped with a stir bar was added photocatalyst IR[DF(CF₃)PPY]₂(DTBPY)PF₆ (5.32 mg, 0.005 mmol), tert-butyl N-[(2S,11S)-6-bromo-2-[[(1S)-3-carbamoyl-1-[[(4-methanesulfonylphenyl)methyl]carbamoyl]propyl]carbamoyl]-12-oxo-1-azatricyclo[6.4.1.0\hat{ }[4,13]]trideca-4(13),5,7-trien-11-yl]carbamate (342.00 mg, 0.475 mmol, Intermediate BP), 3-[5-(6-bromohexyl)-3-methyl-2-oxo-1,3-benzodiazol-1-yl]piperidine-2,6-dione (240.51 mg, 0.569 mmol, Intermediate AU), tris(trimethylsilyl)silane (118.01 mg, 0.475 mmol), and Na2CO3 (150.90 mg, 1.42 mmol). The vial was sealed and placed under nitrogen before 4 mL of DME was added. To a separate vial was added 1,2-dimethoxyethane dihydrochloride nickel (1.04 mg, 0.005 mmol) and dtbbpy (1.27 mg, 0.005 mmol). The catalyst vial was sealed, purged with nitrogen then to it was added 4 mL of DME. The precatalyst solution was sonicated or stirred for 5 min, after which, 4 mL was syringed into the reaction vessel. The solution was degassed by sparging with nitrogen while stirring for 10 minutes before sealing with Parafilm. The reaction was stirred and irradiated with a 34 W blue LED lamp at rt for 4 hrs. On completion, the residue was purified by reverse phase flash chromatography (Column: WelFlash™ C18-I, 20-40 um, 120 g; Eluent A: Water (plus 10 mmol/L FA or 10 mmol/L NH4HCO3); Eluent B: ACN; Gradient: 5%-5% B in 10 min, 20%-60% B in 40 min; Flow rate: 80 mL/min; Detector: 254 nm; desired fractions were collected at 48% B) and concentrated under reduced pressure to afford the title compound (180 mg, 39% yield) as alight yellow solid. ¹H NMR (400 MHz, DMSO-d6) δ 11.10 (s, 1H), 8.56 (t, J=6.0 Hz, 1H), 8.27 (d, J=7.9 Hz, 1H), 7.84 (d, J=8.3 Hz, 2H), 7.46 (d, J=8.3 Hz, 2H), 7.28 (s, 1H), 7.07 (d, J=7.9 Hz, 1H), 7.04-7.02 (m, 1H), 7.00 (d, J=8.0 Hz, 1H), 6.92 (s, 1H), 6.89-6.83 (m, 2H), 6.79 (s, 1H), 5.39-5.28 (m, 1H), 5.13-5.06 (m, 1H), 4.43-4.31 (m, 2H), 4.25-4.17 (m, 1H), 4.07-3.99 (m, 1H), 3.42-3.35 (m, 2H), 3.32 (s, 3H), 3.18 (s, 3H), 3.10-2.98 (m, 2H), 2.98-2.85 (m, 2H), 2.76-2.67 (m, 1H), 2.66-2.58 (m, 3H), 2.49-2.46 (m, 1H), 2.13-2.09 (m, 2H), 2.05-1.96 (m, 3H), 1.95-1.88 (m, 1H), 1.84-1.73 (m, 1H), 1.63-1.49 (m, 4H), 1.39 (s, 9H), 1.35-1.29 (m, 4H); LC/MS (ESI, m/z): [(M+1)]+=983.2.

Step 2—(2S)-2-((3S,6S)-3-amino-9-(6-(1-(2,6-dioxopiperidin-3-yl)-3-methyl-2-oxo-2,3-dihydro-1H-benzo[d]imidazol-5-yl)hexyl)-4-oxo-1,2,3,4,6,7-hexahydroazepino[3,2,1-hi]indole-6-carboxamido)-N1-(4-(methylsulfonyl)benzyl)pentanediamide. To a stirred solution of tert-butyl N-[(2S,11S)-2-[[(1S)-3-carbamoyl-1-[[(4-methanesulfonylphenyl)methyl]carbamoyl]propyl]carbamoyl]-6-[6-[1-(2,6-dioxopiperidin-3-yl)-3-methyl-2-oxo-1,3-benzodiazol-5-yl]hexyl]-12-oxo-1-azatricyclo[6.4.1.0\hat{ }[4,13]]trideca-4(13),5,7-trien-11-yl]carbamate (180.00 mg, 0.183 mmol) in DCM (6.00 mL) was added HCl (gas) in 1,4-dioxane (1.50 mL, 6.00 mmol) dropwise at rt under nitrogen atmosphere. The resulting mixture was stirred for 1 h at rt under nitrogen atmosphere. On completion, the resulting mixture was concentrated under vacuum to afford the title compound (130 mg, 77% yield) as a white solid. 1H NMR (300 MHz, DMSO-d6) δ 11.10 (s, 1H), 8.63 (t, J=5.9 Hz, 1H), 8.52-8.40 (m, 3H), 7.85 (d, J=7.2 Hz, 2H), 7.48 (d, J=8.0 Hz, 2H), 7.34 (s, 1H), 7.05-7.02 (m, 1H), 7.01-6.93 (m, 2H), 6.92-6.76 (m, 3H), 5.41-5.31 (m, 1H), 5.20-5.12 (m, 1H), 4.38 (d, J=5.9 Hz, 2H), 4.27-4.14 (m, 2H), 3.47-3.38 (m, 1H), 3.34 (s, 3H), 3.20 (s, 3H), 3.17-3.04 (m, 2H), 2.99-2.85 (m, 2H), 2.80-2.72 (m, 1H), 2.70-2.57 (m, 4H), 2.49-2.46 (m, 1H), 2.28-2.12 (m, 3H), 2.08-1.97 (m, 2H), 1.95-1.80 (m, 2H), 1.64-1.50 (m, 4H), 1.39-1.28 (m, 4H); LC/MS (ESI, m/z): [(M+1)]+=883.4.

Step 3—(2-(((3S,6S)-6-(((S)-5-amino-1-((4-(methylsulfonyl)benzyl)amino)-1,5-dioxopentan-2-yl)carbamoyl)-9-(6-(1-(2,6-dioxopiperidin-3-yl)-3-methyl-2-oxo-2,3-dihydro-1H-benzo[d]imidazol-5-yl)hexyl)-4-oxo-1,2,3,4,6,7-hexahydroazepino[3,2,1-hi]indol-3-yl)carbamoyl)-1H-indole-5-carbonyl)phosphonic acid. To a stirred mixture of (2S)-2-[[(2S,11S)-11-amino-6-[6-[1-(2,6-dioxopiperidin-3-yl)-3-methyl-2-oxo-1,3-benzodiazol-5-yl]hexyl]-12-oxo-1-azatricyclo[6.4.1.0\hat{ }[4,13]]trideca-4(13),5,7-trien-2-yl]formamido]-N-[(4-methanesulfonylphenyl)methyl]pentanediamide (100.00 mg, 0.113 mmol) and 2-(2,3,4,5,6-pentafluorophenoxycarbonyl)-1H-indole-5-carbonylphosphonic acid (147.85 mg, 0.340 mmol, Intermediate B) in NMP (6.00 mL) was added DBU (86.20 mg, 0.566 mmol) dropwise at rt under nitrogen atmosphere. The resulting mixture was stirred for 3 h at rt under nitrogen atmosphere. On completion, the residue product was purified by reverse phase flash (Column: Spherical C18, 20-40 um, 120 g; Mobile Phase A: Water (0.05% FA), Mobile Phase B: ACN; Flow rate: 45 mL/min; Gradient (B %): 5%˜5%, 4 min; 25%˜55%, 30 min; 95%, 5 min; Detector: 254 nm; Rt: 28 min.) to afford the title compound (38 mg, 30% yield) as a white solid. ¹H NMR (300 MHz, DMSO-d6) δ 12.11 (s, 1H), 11.10 (s, 1H), 8.96 (d, J=8.1 Hz, 1H), 8.83 (s, 1H), 8.54 (t, J=6.1 Hz, 1H), 8.31 (d, J=7.6 Hz, 1H), 8.00-7.93 (m, 1H), 7.88-7.79 (m, 2H), 7.54 (d, J=8.9 Hz, 1H), 7.50-7.41 (m, 3H), 7.29 (s, 1H), 7.06-7.03 (m, 1H), 7.01 (d, J=8.0 Hz, 1H), 6.96 (s, 1H), 6.92-6.84 (m, 2H), 6.79 (s, 1H), 5.41-5.30 (m, 1H), 5.21-5.08 (m, 1H), 4.72-4.63 (m, 1H), 4.43-4.33 (m, 2H), 4.27-4.16 (m, 1H), 3.49-3.42 (m, 3H), 3.34 (s, 3H), 3.18 (s, 3H), 3.16-3.07 (m, 2H), 3.00-2.85 (m, 3H), 2.67-2.57 (m, 4H), 2.26-2.16 (m, 2H), 2.15-2.07 (m, 2H), 2.06-1.95 (m, 2H), 1.94-1.77 (m, 2H), 1.65-1.51 (m, 4H), 1.42-1.32 (m, 4H); LC/MS (ESI, m/z): [(M+1)]+=1134.3.

TABLE 8
Compounds synthesized via Method 6, where the corresponding bromide was coupled with
bromide Intermeduate AU in Step 1; and acid Intermediate AH in Step 3.

| | | LC/MS | |
|---|---|---|---|
| | Step 1 | (ESI, m/z) | |
| I-#ª | Bromide | (M + H)⁺ | 1H NMR (400 MHZ, DMSO-d6) δ |
| I-20^{b} | CK | 852.4 | 12.50-11.83 (m, 1H), 11.09 (s, 1H), 8.95-8.77 (m, 2H), 8.21-7.91 (m, |
| | | | 1H), 7.94-7.71 (m, 1H), 7.48-7.37 (m, 1H), 7.08-6.86 (m, 6H), 5.41- |
| | | | 5.27 (m, 1H), 5.01-4.87 (m, 1H), 4.70-4.53 (m, 1H), 3.38 (s, 3H), |
| | | | 3.16-2.84 (m, 7H), 2.77-2.61 (m, 4H), 2.59 (s, 3H), 2.10-1.93 (m, 2H), |
| | | | 1.64-1.50 (m, 5H), 1.42-1.22 (m, 6H) |
| I-21^{b} | CJ | 965.5 | 12.07 (s, 1H), 11.08 (s, 1H), 8.92-8.87 (m, 1H), 8.86-8.81 (m, 1H), |
| | | | 7.99-7.91 (m, 1H), 7.88-7.81 (m, 1H), 7.57-7.51 (m, 1H), 7.50-7.41 |
| | | | (m, 1H), 7.05-6.99 (m, 2H), 6.98-6.91 (m, 1H), 6.91-6.85 (m, 2H), |
| | | | 5.38-5.31 (m, 1H), 5.01-4.94 (m, 1H), 4.65-4.61 (m, 1H), 3.55-3.44 |
| | | | (m, 4H), 3.15-3.09 (m, 3H), 2.84-2.79 (m, 6H), 2.73-2.69 (m, 4H), |
| | | | 2.62-2.58 (m, 4H), 2.25-2.18 (m, 4H), 2.01-1.94 (m, 1H), 1.59-1.51 |
| | | | (m, 6H), 1.35-1.28 (m, 4H), 1.12-1.01 (m, 3H). |
| I-22^{c} | CH | 951.5 | 12.02 (s, 1H), 11.07 (s, 1H), 9.01-8.85 (m, 1H), 8.84-8.74 (m, 1H), |
| | | | 7.98-7.90 (m, 1H), 7.87-7.78 (m, 1H), 7.66-7.61 (m, 1H), 7.55-7.41 |
| | | | (m, 2H), 7.06-6.84 (m, 5H), 5.37-5.27 (m, 1H), 5.02-4.93 (m, 1H), |
| | | | 4.68-4.64 (m, 1H), 3.66-3.61 (m, 1H), 3.58-3.54 (m, 1H), 3.47-3.41 |
| | | | (m, 4H), 3.25-3.18 (m, 4H), 3.15-3.07 (m, 2H), 2.95-2.80 (m, 2H), |
| | | | 2.75-2.58 (m, 4H), 2.19-2.14 (m, 2H), 2.02-1.94 (m, 3H), 1.70-1.47 |
| | | | (m, 6H), 1.35-1.28 (m, 4H), 1.04-0.99 (m, 3H) |
| I-49^{b} | DV | 937.4 | 11.85 (s, 1H), 9.02-8.84 (m, 2H), 7.97-7.91 (m, 1H), 7.90-7.83 (m, |
| | | | 1H), 7.46-7.36 (m, 3H), 7.21 (s, 1H), 7.05-6.97 (m, 2H), 6.94 (s, 1H), |
| | | | 6.91-6.83 (m, 2H), 6.70 (s, 1H), 5.38-5.29 (m, 1H), 5.04-4.95 (m, 1H), |
| | | | 4.69-4.60 (m, 1H), 3.72-3.61 (m, 1H), 3.51-3.35 (m, 3H), 3.32 (s, 3H), |
| | | | 3.19-3.03 (m, 3H), 2.90-2.80 (m, 2H), 2.76-2.57 (m, 4H), 2.24-2.12 |
| | | | (m, 3H), 2.09-1.95 (m, 4H), 1.63-1.54 (m, 5H), 1.39-1.32 (m, 4H), |
| | | | 1.05-0.98 (m, 3H) |

^{a}In Step 1, Bromide Intermediate AU was coupled with the various bromides listed in the table under photredox conditions. In Step 2, various deprotection methods were employed, including TFA in DCM. In Step 3, acid Intermediate AH was coupled with the amine product from Step 2, unless otherwise stated. Other coupling conditions, such as PyBOP, TEA in DMA at rt were also used for the coupling in Step 3.
^{b}After the final coupling, the product was deprotected with with TFA in ACN or DCM at rt for 1-2 hr. Then the final compound was purified under standard conditions, such as reverse phase chromatography.
^{c}In Step 3, acid Intermediate B was used for the coupling.

### Example 10: Synthesis of (4S)-4-[[(5S,8S,10aR)-6-oxo-5-[5-(phosphonocarbonyl)-1H-indole-2-amido]-3-[(1s,4s)-4-12-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diaz ocin-8-yl]form amido]-4-11 (4-isopropylphenyl)methyl]carbamoyl]butanoic acid (1-96)

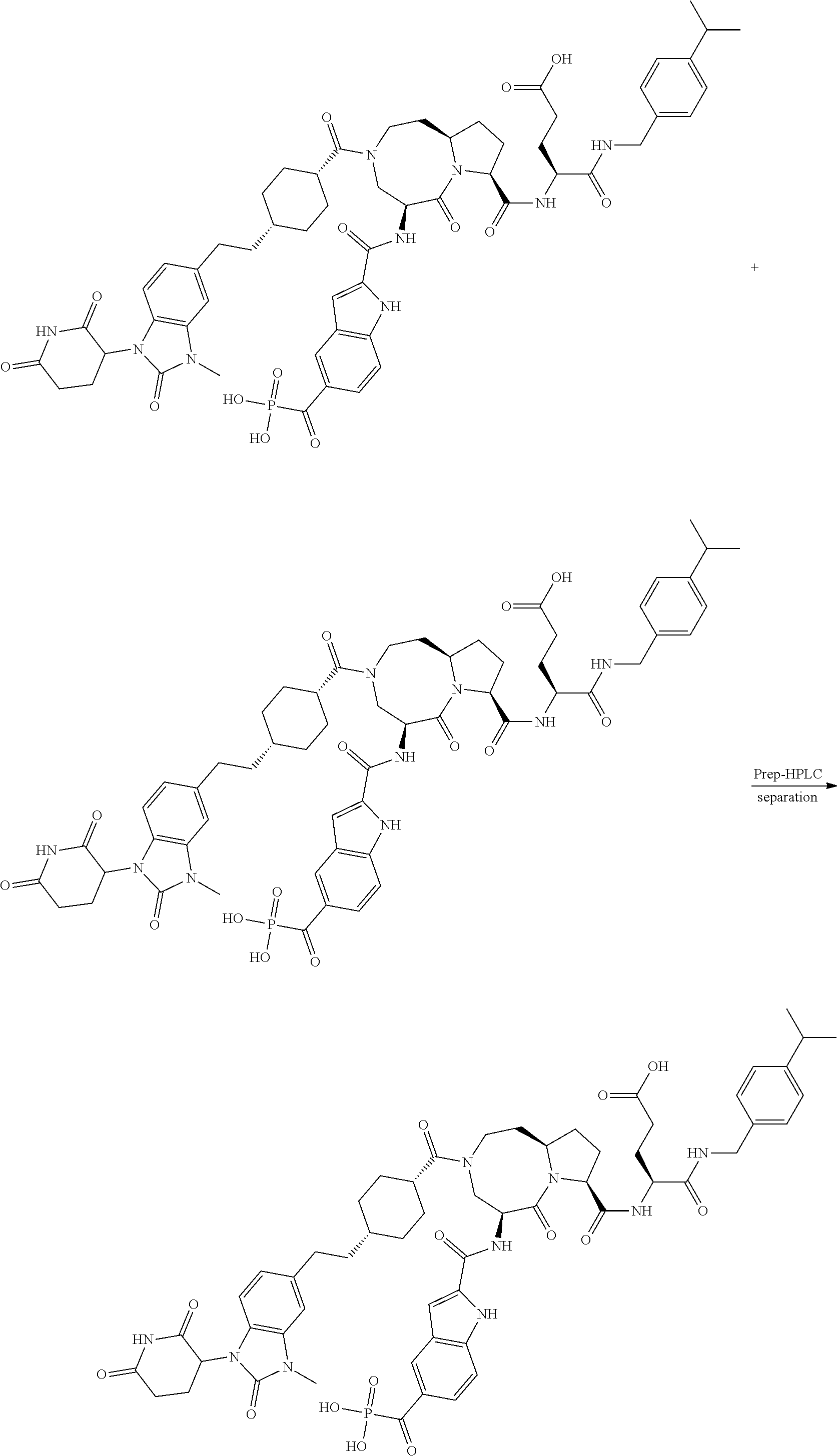

A mixture of (2-(((5S,8S,10aR)-8-(((S)-5-amino-1-((4-isopropylbenzyl)amino)-1,5-dioxopentan-2-yl)carbamoyl)-3-((1r,4R)-4-(2-(1-(2,6-dioxopiperidin-3-yl)-3-methyl-2-oxo-2,3-dihydro-1H-benzo[d]imidazol-5-yl)ethyl)cyclohexane-1-carbonyl)-6-oxodecahydropyrrolo[1,2-a][1,5]diazocin-5-yl)carbamoyl)-1H-indole-5-carbonyl)phosphonic acid and (4S)-4-((5S,8S,10aR)-3-((1r,4R)-4-(2-(1-(2,6-dioxopiperidin-3-yl)-3-methyl-2-oxo-2,3-dihydro-1H-benzo[d]imidazol-5-yl)ethyl)cyclohexane-1-carbonyl)-6-oxo-5-(5-(phosphonocarbonyl)-1H-indole-2-carboxamido)decahydropyrrolo [1,2-a][1,5]diazocine-8-carboxamido)-5-((4-isopropylbenzyl)amino)-5-oxopentanoic acid (4 g, Example I-59) was separated by Prep-HPLC with the following conditions: Column: Atlantis Prep T3 OBD Column, 30×150 mm, 5 um; Mobile Phase A: 10 mM aq NH4Ac; Mobile Phase B: ACN; Flow rate: 30 mL/min; Gradient: 40% B to 80% B in 11.5 min; Detector: UV 254 & 210 nm; Rt: 7.8 min. The product-containing fractions were combined and lyophilized overnight to afford (4S)-4-[[(5S,8S,10aR)-6-oxo-5-[5-(phosphonocarbonyl)-1H-indole-2-amido]-3-[(1s,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-8-yl]formamido]-4-[[(4-isopropylphenyl)methyl]carbamoyl]butanoic acid (413 mg, 10% yield) as a white solid. 1H NMR (400 MHz, DMSO-d6) δ 12.12-12.05 (m, 1H), 11.09-11.04 (m, 1H), 8.80 (s, 1H), 8.47-8.42 (m, 1H), 8.35-8.29 (m, 1H), 8.24-8.20 (m, 1H), 7.99-7.95 (m, 1H), 7.57-7.53 (m, 1H), 7.45-7.41 (m, 1H), 7.18-7.14 (m, 4H), 7.05 (s, 1H), 7.03-6.98 (m, 1H), 6.91-6.87 (m, 1H), 5.34 (dd, J=12.7, 5.3 Hz, 1H), 5.05-4.97 (m, 1H), 4.47-4.43 (m, 1H), 4.31-4.14 (m, 4H), 3.93-3.87 (m, 1H), 3.55-3.48 (m, 3H), 3.25-3.07 (m, 3H), 3.01-2.96 (m, 1H), 2.91-2.84 (m, 2H), 2.77-2.65 (m, 1H), 2.65-2.61 (m, 4H), 2.32-2.26 (m, 2H), 2.21-2.18 (m, 1H), 2.03-1.99 (m, 2H), 1.94-1.90 (m, 1H), 1.87-1.76 (m, 1H), 1.72-1.47 (m, 17H), 1.17 (d, J=6.9 Hz, 6H). LC/MS (ESI, m/z): [(M+H)]+=1134.3.

### Example 11: Synthesis of 2-[[(2S, 11S)-2-(dimethylcarbamoyl)-6-[6-[1-(2, 6-dioxopiperidin-3-yl)-3-methyl-2-oxo-1, 3-benzodiazol-5-yl] hexyl]-12-oxo-1-azatricyclo [6.4.1.0\hat{ }[4, 13]] trideca-4(13), 5, 7-trien-11-yl] carbamoyl]-1H-indole-5-carbonylphosphonic acid (I-25)

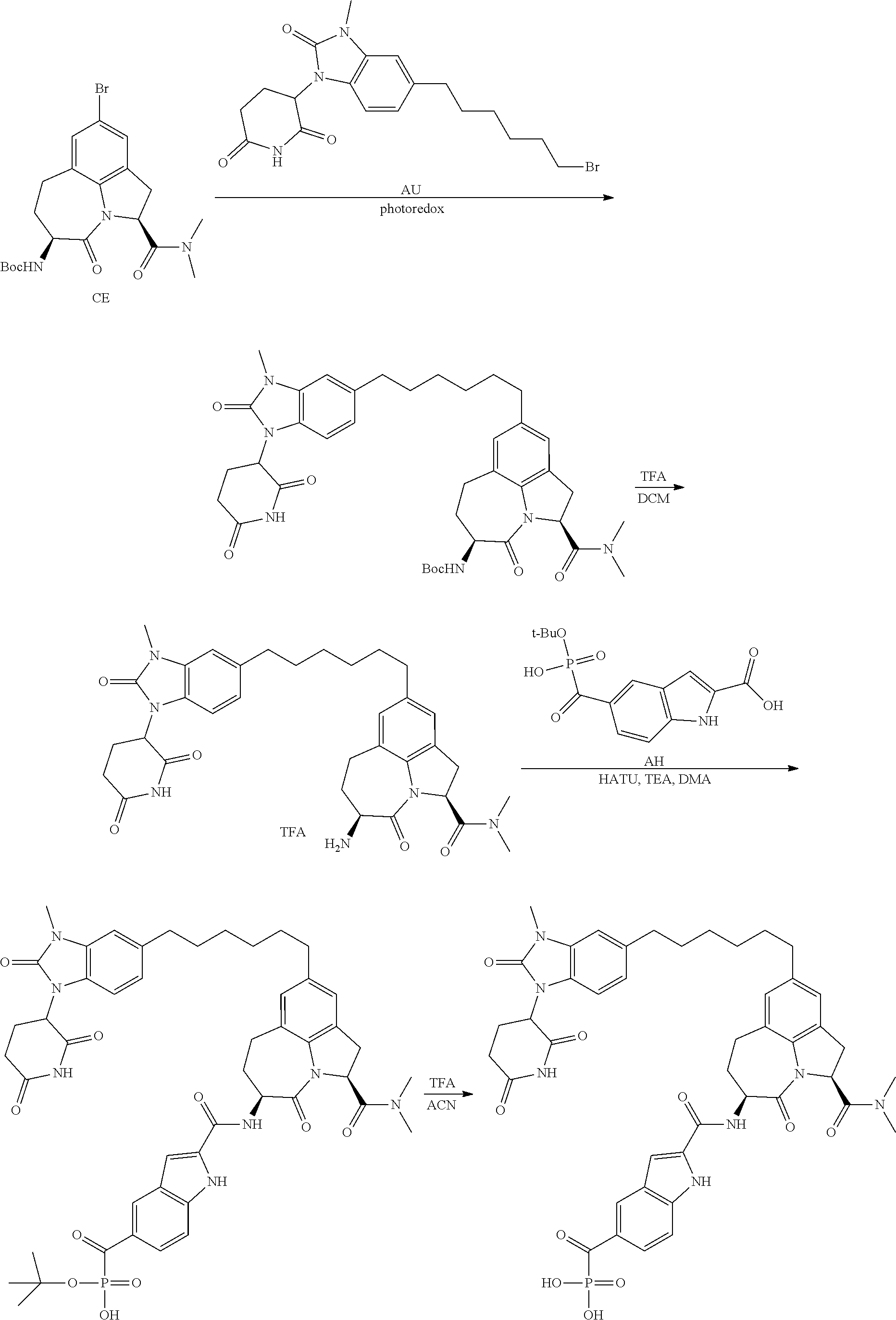

Step 1—Tert-butyl N-[(2S)-4-carbamoyl-1-(2-chloro-3-[5-[1-(2, 6-dioxopiperidin-3-yl)-3-methyl-2-oxo-1, 3-benzodiazol-5-yl] pentyl] phenoxy) butan-2-yl] carbamate. To an 8 ml vial equipped with a stir bar was added photocatalyst IR[DF(CF₃)PPY]₂(DTBPY)PF₆ (101.69 mg, 0.091 mmol), tert-butyl N-[(2S,11S)-6-bromo-2-(dimethylcarbamoyl)-12-oxo-1-azatricyclo [6.4.1.0\hat{ }[4,13]] trideca-4(13),5,7-trien-11-yl] carbamate (820.00 mg, 1.81 mmol, Intermediate CE), 3-[5-(6-bromohexyl)-3-methyl-2-oxo-1,3-benzodiazol-1-yl]piperidine-2,6-dione (765.57 mg, 1.813 mmol, Intermediate AU), tris(trimethylsilyl)silane (450.76 mg, 1.813 mmol), and Na2CO3 (576.39 mg, 5.44 mmol,). The vial was sealed and placed under nitrogen before 4 ml of DME was added. To a separate vial was added 1, 2-dimethoxyethane dihydrochloride nickel (19.91 mg, 0.091 mmol) and dtbbpy (24.33 mg, 0.091 mmol). The catalyst vial was sealed, purged with nitrogen then to it was added 8 ml of DME. The precatalyst solution was sonicated or stirred for 5 min, after which, 4 ml was syringed into the reaction vessel. The solution was degassed by sparging with nitrogen while stirring for 10 minutes before sealing with Parafilm. The reaction was stirred and irradiated with a 34 W blue LED lamp (with cooling fan to keep the reaction temperature at 25° C.) for 3 hours. On completion, the resulting mixture was concentrated under reduced pressure. The residue was purified by reverse phase flash chromatography (Column: WelFlash™ C18-I, 20-40 m, 120 g; EluentA: Water (plus 10 mmol/L FA); Eluent B: ACN; Gradient: 40%-70% B in 30 min; Flow rate: 60 mL/min; Detector: 220/254 nm; desired fractions were collected at 53% B) and concentrated under reduced pressure to afford the title compound (491.8 mg, 38% yield) as an white solid. ¹H NMR (300 MHz, Chloroform-d) δ 8.37-8.31 (m, 1H), 6.90-6.79 (m, 4H), 6.73-6.69 (m, 1H), 5.52-5.45 (m, 1H), 5.24-5.17 (m, 1H), 4.28-4.19 (m, 1H), 3.42 (s, 3H), 3.32-3.19 (m, 1H), 3.16 (s, 3H), 3.12-3.02 (m, 1H), 2.96 (s, 3H), 2.91-2.81 (m, 2H), 2.78-2.59 (m, 4H), 2.53-2.45 (m, 2H), 2.35-2.16 (m, 4H), 1.65-1.51 (m, 4H), 1.45 (s, 9H), 1.38-1.30 (m, 4H); LC/MS (ESI, m/z): [(M+H)]+=715.5.

Step 2—(2S, 11S)-11-amino-6-[6-[1-(2, 6-dioxopiperidin-3-yl)-3-methyl-2-oxo-1, 3-benzodiazol-5-yl] hexyl]-N, N-dimethyl-12-oxo-1-azatricyclo [6.4.1.0\hat{ }[4, 13]] trideca-4(13), 5, 7-triene-2-carboxamide trifluoroacetate. To a stirred mixture of tert-butyl N-[(2S,11S)-2-(dimethylcarbamoyl)-6-[6-[1-(2,6-dioxopiperidin-3-yl)-3-methyl-2-oxo-1,3-benzodiazol-5-yl] hexyl]-12-oxo-1-azatricyclo [6.4.1.0\hat{ }[4,13]] trideca-4(13),5,7-trien-11-yl] carbamate (200.00 mg, 0.280 mmol) in DCM (5.00 mL) was added a solution of 4 M TFA (2.00 mL, 26.93 mmol) at 0° C. under nitrogen atmosphere. The resulting mixture was then stirred for 2 h at 25° C. under nitrogen atmosphere. On completion, the reaction mixture was concentrated under reduced pressure. The residue was then triturated with Et2O to afford the title compound (166.6 mg, 84% yield) as an off-white solid. ¹H NMR (300 MHz, DMF-d7) δ 7.02-6.96 (m, 2H), 6.94-6.86 (m, 3H), 5.60-5.53 (m, 1H), 5.34-5.27 (m, 1H), 4.17-4.10 (m, 1H), 3.39 (s, 3H), 3.31-3.30 (m, 3H), 3.20 (s, 3H), 2.97-2.95 (m, 1H), 2.93-2.80 (m, 3H), 2.80-2.61 (m, 4H), 2.56-2.46 (m, 2H), 2.45-2.09 (m, 4H), 1.66-1.52 (m, 4H), 1.37-1.31 (m, 4H); LC/MS (ESI, m/z): [(M+H)]+=615.4.

Step 3—Tert-butoxy (2-[[(2S, 11S)-2-(dimethylcarbamoyl)-6-[6-[1-(2,6-dioxopiperidin-3-yl)-3-methyl-2-oxo-1, 3-benzodiazol-5-yl] hexyl]-12-oxo-1-azatricyclo [6.4.1.0\hat{ }[4, 13]] trideca-4(13), 5, 7-trien-11-yl] carbamoyl]-1H-indole-5-carbonyl) phosphinic acid. To a stirred solution of (2S,11S)-11-amino-6-[6-[1-(2,6-dioxopiperidin-3-yl)-3-methyl-2-oxo-1,3-benzodiazol-5-yl] hexyl]-N,N-dimethyl-12-oxo-1-azatricyclo [6.4.1.0\hat{ }[4,13]] trideca-4(13),5,7-triene-2-carboxamide trifluoroacetate (180.00 mg, 0.253 mmol) and 5-[[tert-butoxy (hydroxy)phosphoryl]carbonyl]-1H-indole-2-carboxylic acid (90.35 mg, 0.278 mmol, Intermediate AH) in DMA (5.00 mL) were added TEA (0.108 mL, 0.758 mmol) and HATU (115.23 mg, 0.303 mmol) at 0° C. under nitrogen atmosphere. The resulting mixture was stirred for 1 h at 25° C. under nitrogen atmosphere. On completion, the reaction mixture was concentrated under reduced pressure. The residue was purified by reverse flash chromatography (Column: WelFlash™ C18-I, 20-40 m, 120 g; Eluent A: Water (plus 10 mmol/L FA); Eluent B: ACN; Gradient: 30%-50% B in 30 min; Flow rate: 60 mL/min; Detector: 220/254 nm; desired fractions were collected at 36% B) and concentrated under reduced pressure to afford the title compound (105 mg, 45% yield) as a white solid. ¹H NMR (300 MHz, DMSO-d6) δ 8.35 (s, 2H), 7.96-7.91 (m, 1H), 7.03-6.98 (m, 2H), 6.92-6.84 (m, 4H), 5.47 (m, 1H), 5.34 (m, 1H), 4.68-4.62 (m, 1H), 3.56-3.47 (m, 1H), 3.34-3.31 (m, 3H), 3.27-3.14 (m, 2H), 3.09 (s, 3H), 3.06-2.87 (m, 2H), 2.80 (s, 3H), 2.77-2.58 (m, 6H), 2.25-1.97 (m, 4H), 1.63-1.53 (m, 4H), 1.35-1.33 (m, 2H), 1.29 (s, 9H), 1.24 (s, 2H); LC/MS (ESI, m/z): [(M+H)]+=922.5.

Step 4—2-[[(2S, 11S)-2-(dimethylcarbamoyl)-6-[6-[1-(2, 6-dioxopiperidin-3-yl)-3-methyl-2-oxo-1, 3-benzodiazol-5-yl] hexyl]-12-oxo-1-azatricyclo [6.4.1.0\hat{ }[4, 13]] trideca-4(13), 5, 7-trien-11-yl] carbamoyl]-1H-indole-5-carbonylphosphonic acid. To a stirred solution of tert-butoxy(2-[[(2S,11S)-2-(dimethylcarbamoyl)-6-[6-[1-(2,6-dioxopiperidin-3-yl)-3-methyl-2-oxo-1,3-benzodiazol-5-yl] hexyl]-12-oxo-1-azatricyclo [6.4.1.0\hat{ }[4,13]] trideca-4(13),5,7-trien-11-yl] carbamoyl]-1H-indole-5-carbonyl) phosphinic acid (105.10 mg, 0.114 mmol) in ACN (5.00 mL) was added TFA (2.00 mL) dropwise at rt under nitrogen atmosphere. The resulting mixture was stirred for 2 h at rt under nitrogen atmosphere. On completion, the reaction mixture was concentrated under reduced pressure. The residue was purified by reverse flash chromatography (Column: WelFlash™ C18-I, 20-40 μm, 120 g; Eluent A: Water (plus 10 mmol/L FA); Eluent B: ACN; Gradient: 20%-50% B in 30 min; Flow rate: 60 mL/min; Detector: 220/254 nm; desired fractions were collected at 33% B) and concentrated under reduced pressure to afford the title compound (36.5 mg, 37% yield) as a white solid. 1H NMR (300 MHz, DMSO-d6) δ 12.06 (s, 1H), 11.07 (s, 1H), 8.97-8.75 (m, 2H), 8.04-7.88 (m, 1H), 7.59-7.41 (m, 2H), 7.11-6.96 (m, 2H), 6.97-6.78 (m, 3H), 5.54-5.42 (m, 1H), 5.40-5.27 (m, 1H), 4.77-4.57 (m, 1H), 3.09 (s, 3H), 3.06-2.88 (m, 6H), 2.88-2.70 (m, 7H), 2.70-2.55 (m, 4H), 2.32-1.94 (m, 4H), 1.70-1.46 (m, 4H), 1.42-1.15 (m, 5H); LC/MS (ESI, m/z): [(M+H)]+=866.4.

### Example 12: Synthesis of 2-[[(5S,8S,10aR)-8-[[(1S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl]methyl]cyclohexyl]ethyl]-octahydropyrrolo[1,2-a][1,5]diazocin-5-yl]carbamoyl]-1H-indole-5-carbonylphosphonic acid (I-52)

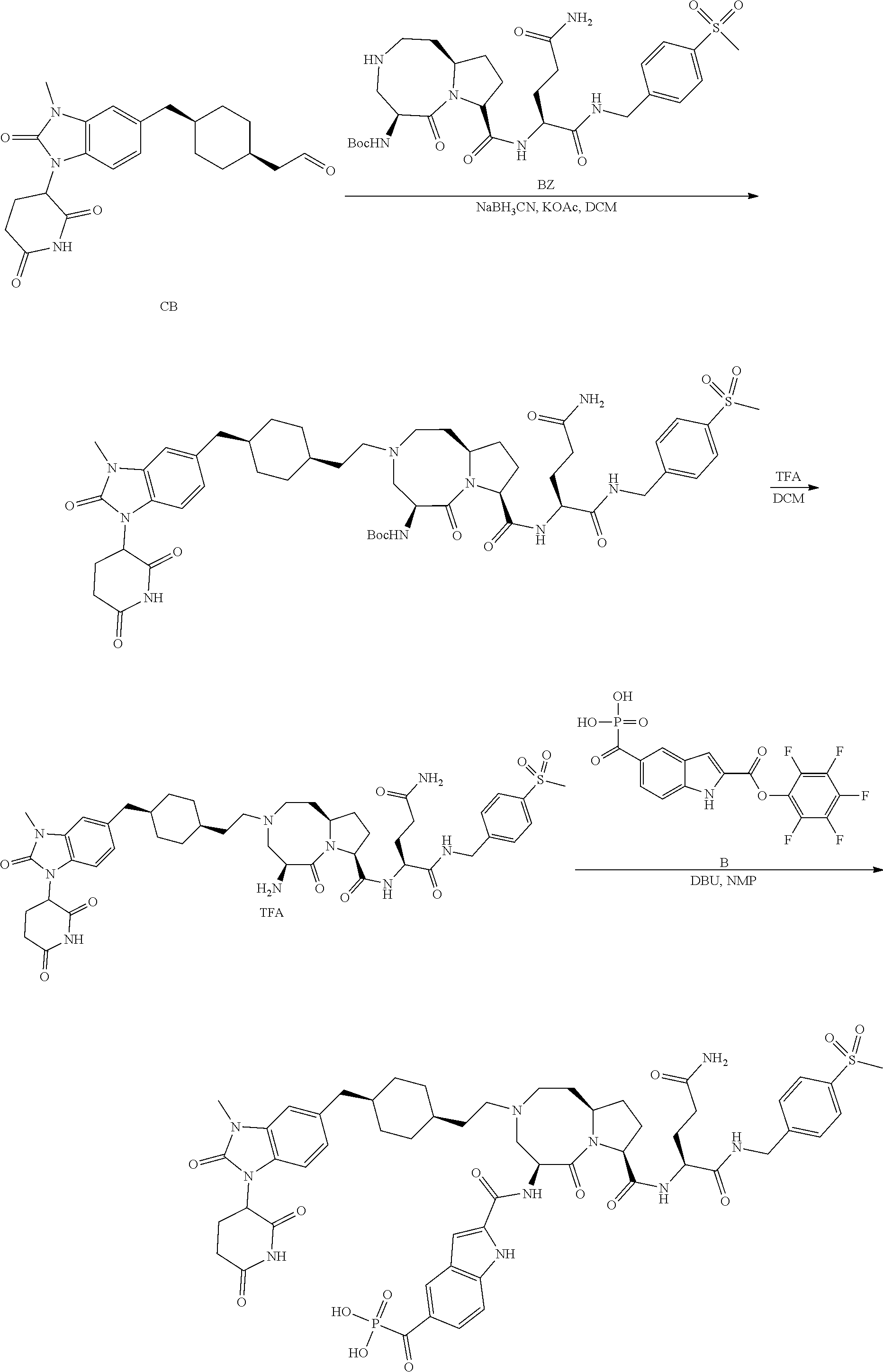

Step 1—tert-butyl N-[(5S,8S,10aR)-8-[[(1S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] ethyl]-octahydropyrrolo[1,2-a][1,5]diazocin-5-yl]carbamate. To a stirred solution of 2-[(1s,4s)-4-[[1-(2,6-dioxopiperidin-3-yl)-3-methyl-2-oxo-1,3-benzodiazol-5-yl]methyl]cyclohexyl]acetaldehyde (40.00 mg, 0.101 mmol, Intermediate CB) and tert-butyl N-[(5S,8S,10aR)-8-[[(1S)-3-carbamoyl-1-[[(4-methanesulfonylphenyl)methyl]carbamoyl]propyl]carbamoyl]-6-oxo-octahydro-1H-pyrrolo[1,2-a][1,5]diazocin-5-yl]carbamate (68.94 mg, 0.111 mmol, Intermediate BZ) in i-PrOH (1.50 mL) and DCM (0.50 mL) were added NaBH3CN (12.65 mg, 0.202 mmol) and KOAc (29.63 mg, 0.303 mmol) in portions at rt under nitrogen atmosphere and the mixture was stirred for 1 h at room temperature under air atmosphere. On completion, the residue was purified by reverse phase Flash chromatography (Column: WelFlash™ C18-I, 20-40 um, 120 g; Eluent A: Water (plus 10 mmol/L NH4HCO3); Eluent B: ACN; Gradient: 30%-60% B in 25 min; Flow rate: 60 mL/min; Detector: 220/254 nm; desired fractions were collected at 45% B) and concentrated under reduced pressure to afford the title compound (80 mg, 79% yield) as a white solid. ¹H NMR (300 MHz, Chloroform-d) δ 7.92-7.88 (m, 2H), 7.72-7.69 (m, 1H), 7.56-7.52 (m, 3H), 6.85-6.71 (m, 5H), 5.93-5.89 (m, 1H), 5.43-5.39 (m, 1H), 5.21-5.18 (m, 1H), 5.12-5.08 (m, 1H), 4.79-4.76 (m, 1H), 4.65-4.61 (m, 2H), 4.49-4.45 (m, 1H), 4.37-4.32 (m, 2H), 3.45-3.41 (m, 3H), 3.05-3.02 (m, 3H), 2.93-2.88 (m, 2H), 2.85-2.68 (m, 6H), 2.65-2.58 (m, 3H), 2.47-2.44 (m, 2H), 2.38-2.35 (m, 2H), 2.28-2.17 (m, 3H), 2.02-1.98 (m, 12H), 1.78-1.75 (m, 3H), 1.50-1.47 (m, 9H); L C/MS (ESI, m/z): [(M+H)]+=1004.5.

Step 2—(2S)-2-[[(5S,8S,10aR)-5-amino-6-oxo-3-[2-[(1s,4s)-4-[[1-(2,6-dioxopiperidin-3-yl)-3-methyl-2-oxo-1,3-benzodiazol-5-yl]methyl] cyclohexyl] ethyl]-octahydropyrrolo [1,2-a][1,5]diazocin-8-yl]formamido]-N-[(4-methanesulfonylphenyl)methyl]pentanediamide; trifluoroacetate. To a stirred solution of tert-butyl N-[(5 S,8 S,10 aR)-8-[[(1 S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] ethyl]-octahydropyrrolo[1,2-a][1,5]diazocin-5-yl]carbamate (80.00 mg) in DCM (3.00 mL) was added trifluoroacetaldehyde (1.00 mL) dropwise at rt under air atmosphere. The resulting mixture was stirred for 2 h at rt under air atmosphere. On completion, the reaction mixture was concentrated under reduced pressure to give the title compound (80 mg, 90% yield) as a light brown solid. ¹H NMR (300 MHz, DMSO-d6) δ 11.06 (m, 1H), δ 8.58-8.54 (m, 3H), 7.88-7.84 (m, 2H), 7.49-7.46 (m, 5H), 7.32-7.38 (m, 5H), 7.17-7.14 (m, 5H), 7.02-6.97 (m, 2H), 6.83-6.79 (m, 2H), 5.34-5.28 (m, 1H), 4.41-4.39 (m, 1H), 4.29-4.25 (m, 1H), 3.38-3.35 (m, 2H), 3.17-3.12 (m, 2H), 2.87-2.85 (m, 1H), 2.67-2.63 (m, 1H), 2.57-2.52 (m, 2H), 2.13-2.10 (m, 1H), 2.02-1.96 (m, 3H), 1.80-1.76 (m, 6H), 1.47 (m, 1H), 1.50-1.20 (m, 14H); LC/MS (ESI, m/z): [(M+H)]+=904.4.

Step 3—2-[[(5S,8S,10aR)-8-[[(1S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl] methyl] cyclohexyl] ethyl]-octahydropyrrolo[1,2-a][1,5]diazocin-5-yl]carbamoyl]-1H-indole-5-carbonylphosphonic acid. To a stirred solution of (2S)-2-[[(5S,8S,10aR)-5-amino-6-oxo-3-[2-[(1s,4s)-4-[[1-(2,6-dioxopiperidin-3-yl)-3-methyl-2-oxo-1,3-benzodiazol-5-yl]methyl]cyclohexyl]ethyl]-octahydropyrrolo [1,2-a][1,5]diazocin-8-yl]formamido]-N-[(4-methanesulfonylphenyl)methyl]pentanediamide trifluoroacetate (20.00 mg, 0.020 mmol) and 2-(2,3,4,5,6-pentafluorophenoxycarbonyl)-1H-indole-5-carbonylphosphonic acid (26.06 mg, 0.060 mmol, Intermediate B) in NMP (2.00 mL) were added DBU (15.19 mg, 0.100 mmol) dropwise at rt under nitrogen atmosphere. The resulting mixture was stirred for 1 h at rt under air atmosphere. On completion, the reaction mixture was purified directly by reverse phase flash chromatography (Column: WelFlash™ C18-I, 20-40 um, 120 g; Eluent A: Water (plus 10 mmol/L NH4HCO3); Eluent B: ACN; Gradient: 15%-45% B in 25 min; Flow rate: 60 mL/min; Detector: 220/254 nm; desired fractions were collected at 31% B) and concentrated under reduced pressure to afford the title compound (5.5 mg, 6% yield) as a white solid. ¹H NMR (400 MHz, DMSO-d6) δ 12.05 (s, 1H), 11.09 (s, 1H), 8.85 (s, 1H), 8.43-8.39 (m, 1H), 8.01-7.94 (m, 1H), 7.87-7.85 (m, 3H), 7.54-7.47 (m, 4H), 7.45-7.40 (m, 1H), 7.31-7.27 (m, 1H), 7.02-6.94 (m, 2H), 6.87-6.78 (m, 3H), 5.35-5.31 (m, 1H), 4.51-4.34 (m, 2H), 4.34-4.31 (m, 2H), 3.32-3.29 (m, 4H), 3.16 (s, 3H), 2.95-2.90 (m, 1H), 2.74-2.55 (m, 4H), 2.35-2.23 (m, 1H), 2.19-2.08 (m, 3H), 2.03-1.98 (m, 1H), 1.94-1.90 (m, 1H), 1.87-1.66 (m, 13H), 1.43-1.39 (m, 9H), 1.32-1.23 (m, 3H); LC/MS (ESI, m/z): [(M+H)]+=1155.3.

### Example 13: Synthesis of 2-[[(5S,8S,10aR)-8-[[(1S)-3-carbamoyl-1-[[(4-methanesulfonylphenyl)methyl] carbamoyl] propyl] carbamoyl]-6-oxo-3-[(1r,4s)-4-[2-[1-(2,6-dioxopiperidin-3-yl)-3-methyl-2-oxo-1,3-benzodiazol-5-yl] ethyl] cyclohexanecarbonyl]-octahydropyrrolo[1,2-a][1,5]diazocin-5-yl]carbamoyl]-1H-indole-5-carbonylphosphonic acid (I-68)

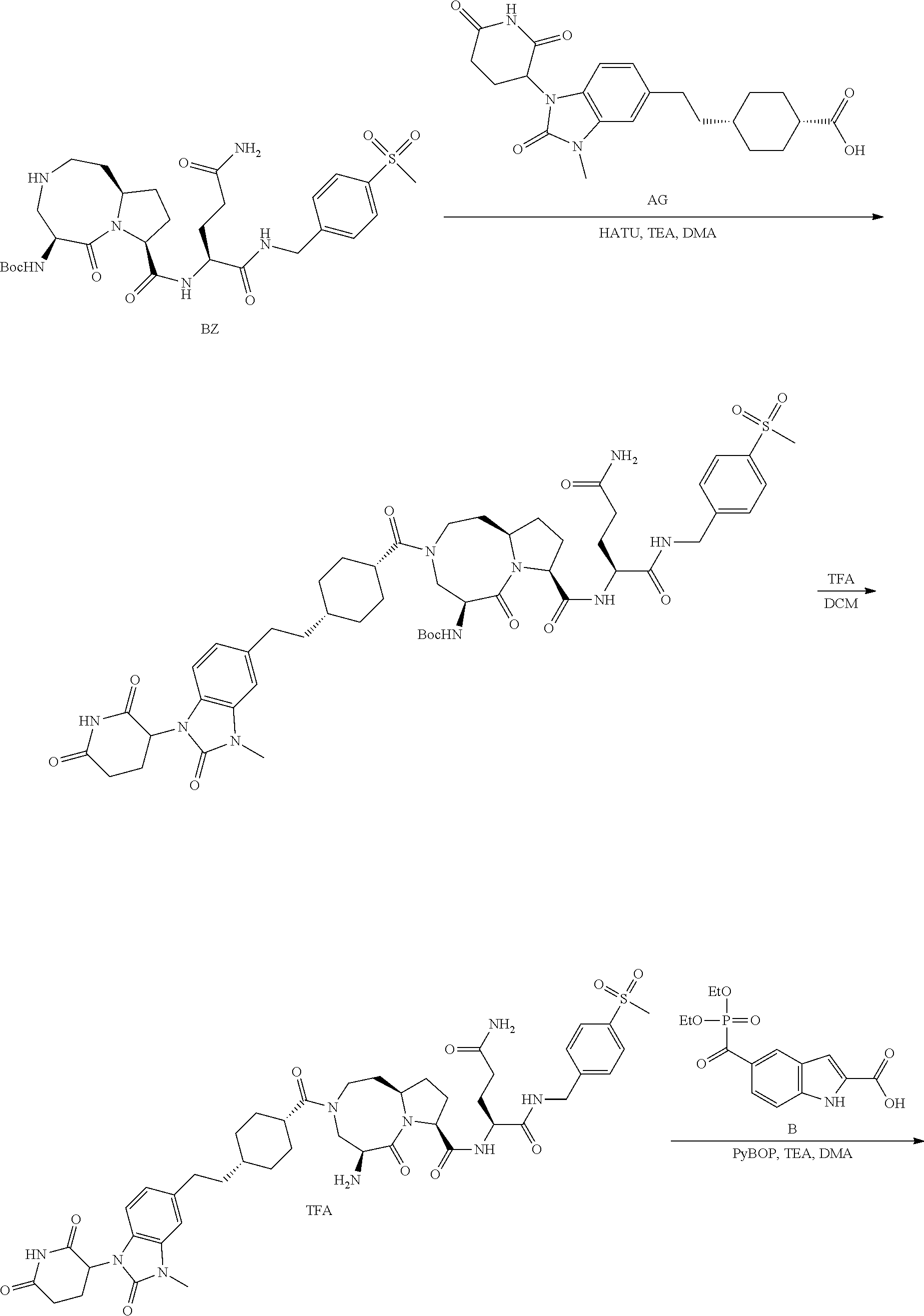

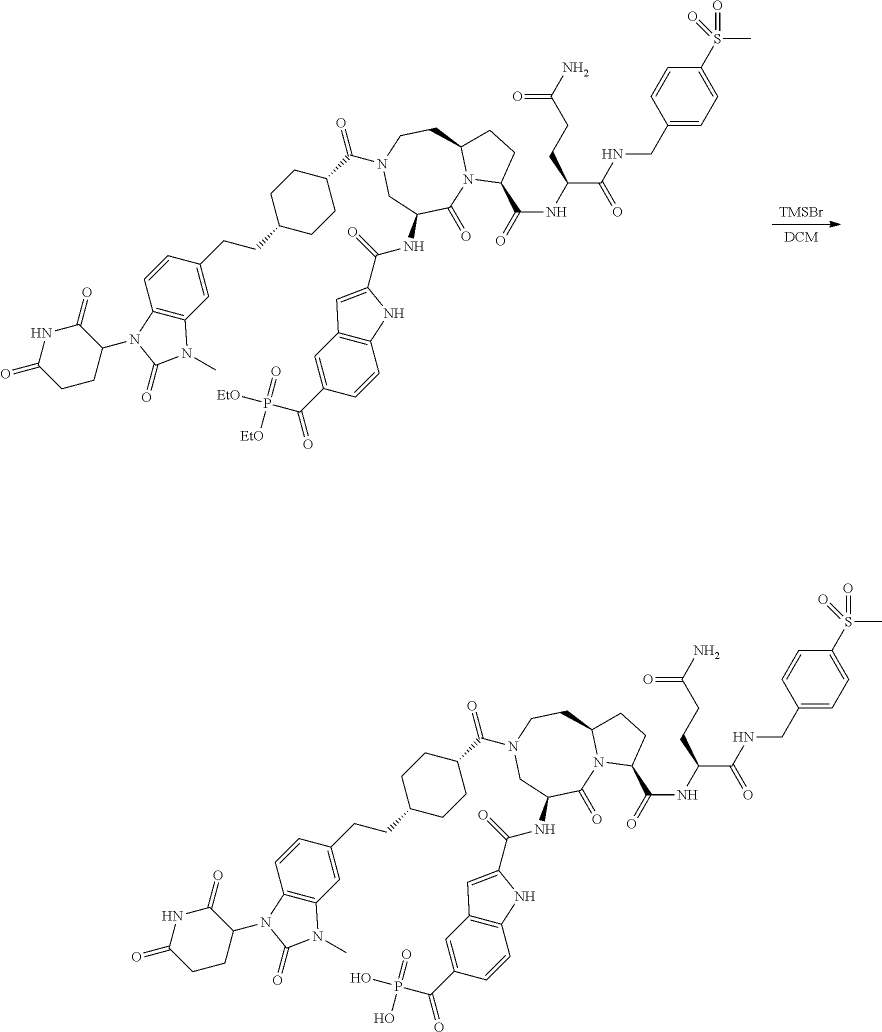

Step 1—Tert-butyl N-[(5S,8S,10aR)-8-[[(1S)-3-carbamoyl-1-[[(4-methanesulfonylphenyl)methyl]carbamoyl]propyl]carbamoyl]-6-oxo-3-[(1R,4S)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamate. To a solution of tert-butyl N-[(5S,8S,10aR)-8-[[(1S)-3-carbamoyl-1-[[(4-methanesulfonylphenyl)methyl]carbamoyl]propyl]carbamoyl]-6-oxo-octahydro-1H-pyrrolo[1,2-a][1,5]diazocin-5-yl]carbamate (450.00 mg, 0.723 mmol, Intermediate BZ) and (1R,4S)-4-[2-[1-(2,6-dioxopiperidin-3-yl)-3-methyl-2-oxo-1,3-benzodiazol-5-yl]ethyl]cyclohexane-1-carboxylic acid (298.78 mg, 0.723 mmol, Intermediate AG) in DMA (10.00 mL) were added TEA (0.301 mL, 2.17 mmol) and HATU (329.71 mg, 0.867 mmol) at 25° C. The resulting mixture was stirred for overnight at rt under nitrogen atmosphere. On completion, the reaction mixture was purified directly by reverse phase flash chromatography (Column: WelFlash™ C18-I, 330 g; EluentA: Water (plus 10 mmol/L FA); Eluent B: ACN; Gradient: 30%-50% B in 20 min; Flow rate: 80 mL/min; Detector: 220/254 nm; desired fractions were collected at 43% B) and concentrated under reduced pressure to afford the title compound (517 mg, 70% yield) as a white solid. ¹H NMR (300 MHz, Chloroform-d) δ 9.36 (d, J=15.8 Hz, 1H), 7.84 (d, J=8.0 Hz, 3H), 7.42 (d, J=8.1 Hz, 3H), 7.01 (d, J=8.1 Hz, 1H), 6.92-6.74 (m, 3H), 6.53 (s, 1H), 6.04-5.90 (m, 1H), 5.88-5.67 (m, 1H), 5.29-5.15 (m, 1H), 5.08-4.99 (m, 1H), 4.59-4.40 (m, 4H), 4.12-3.85 (m, 2H), 3.39 (s, 3H), 3.22-2.96 (m, 5H), 2.90-2.69 (m, 2H), 2.68-2.55 (m, 3H), 2.54-2.36 (m, 2H), 2.34-2.09 (m, 6H), 1.91-1.80 (m, 2H), 1.79-1.49 (m, 13H), 1.44 (s, 9H); LC/MS (ESI, m/z): [(M+H)]+=1018.5.

Step 2—(2S)-2-[[(5S,8S,10aR)-5-amino-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-8-yl]formamido]-N-[(4-methanesulfonylphenyl)methyl]pentanediamide trifluoroacetate. To a stirred solution of tert-butyl N-[(5S,8S,10aR)-8-[[(1S)-3-carbamoyl-1-[[(4-methanesulfonylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamate (517.00 mg, 0.508 mmol) in DCM (10.00 mL) was added TFA (2.00 mL) dropwise at rt under nitrogen atmosphere. The resulting mixture was stirred for 1 h at rt under nitrogen atmosphere. On completion, the reaction mixture was concentrated under reduced pressure to afford the title compound (490 mg) as a white solid. ¹H NMR (400 MHz, DMSO-d6) δ 11.09 (s, 1H), 8.59 (d, J=6.2 Hz, 1H), 8.41-8.26 (m, 3H), 7.87 (d, J=8.1 Hz, 2H), 7.53-7.47 (m, 2H), 7.24 (s, 1H), 7.07-6.98 (m, 2H), 6.90-6.85 (m, 1H), 6.80 (s, 1H), 5.35 (dd, J=12.6, 5.4 Hz, 1H), 4.90-4.83 (m, 1H), 4.46-4.44 (m, 1H), 4.41-4.39 (m, 1H), 4.28-4.24 (m, 1H), 4.20-4.16 (m, 1H), 3.99-3.88 (m, 1H), 3.87-3.75 (m, 1H), 3.33 (s, 3H), 3.20 (s, 3H), 2.93-2.85 (m, 1H), 2.73-2.59 (m, 5H), 2.42-2.32 (m, 1H), 2.26-2.18 (m, 2H), 2.16-2.09 (m, 2H), 2.04-1.87 (m, 4H), 1.84-1.70 (m, 6H), 1.67-1.43 (m, 10H); LC/MS (ESI, m/z): [(M+H)]+=918.3.

Step 3—Diethyl 2-[[(5S,8S,10aR)-8-[[(1S)-3-carbamoyl-1-[[(4-methanesulfonylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonylphosphonate. To a solution of (2S)-2-[[(5S,8S,10aR)-5-amino-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-8-yl]formamido]-N-[(4-methanesulfonylphenyl)methyl]pentanediamide trifluoroacetate (490.00 mg, 0.482 mmol), 5-[(diethoxyphosphoryl)carbonyl]-1H-indole-2-carboxylic acid (156.85 mg, 0.482 mmol, Intermediate B) in DMA (7.00 mL) were added TEA (0.201 mL, 1.45 mmol) and PyBOP (301.14 mg, 0.579 mmol) at 25° C. The resulting mixture was stirred for 1 h at rt under nitrogen atmosphere. On completion, the reaction mixture was purified directly by reverse phase flash chromatography (Column: WelFlash™ C18-I, 330 g; EluentA: Water (plus 10 mmol/L FA); Eluent B: ACN; Gradient: 30%-50% B in 20 min; Flow rate: 80 mL/min; Detector: 220/254 nm; desired fractions were collected at 42% B) and concentrated under reduced pressure to afford the title compound (420 mg, 71% yield) as a white solid. ¹H NMR (300 MHz, DMSO-d6) δ 12.17-12.01 (m, 1H), 11.08 (s, 1H), 8.87-8.71 (m, 1H), 8.57 (d, J=5.9 Hz, 1H), 8.55-8.41 (m, 1H), 8.32 (q, J=11.5, 10.2 Hz, 1H), 7.95 (dd, J=8.8, 1.7 Hz, 1H), 7.86 (d, J=8.1 Hz, 2H), 7.65-7.55 (m, 1H), 7.54-7.47 (m, 3H), 7.32-7.17 (m, 1H), 7.08-6.91 (m, 2H), 6.91-6.76 (m, 2H), 5.34 (dd, J=12.6, 5.6 Hz, 1H), 5.04-4.91 (m, 1H), 4.89-4.75 (m, 1H), 4.47-4.35 (m, 3H), 4.31-4.10 (m, 7H), 3.94 (d, J=13.5 Hz, 1H), 3.87-3.71 (m, 1H), 3.46 (d, J=2.2 Hz, 1H), 3.33 (s, 3H), 3.18 (s, 3H), 3.06-2.81 (m, 2H), 2.78-2.54 (m, 4H), 2.28-2.10 (m, 4H), 2.01-1.78 (m, 6H), 1.72-1.45 (m, 11H), 1.29 (t, J=7.0 Hz, 6H); LC/MS (ESI, m/z): [(M+H)]+=1225.5.

Step 4—2-[[(5S,8S,10aR)-8-[[(1S)-3-carbamoyl-1-[[(4-methanesulfonylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonylphosphonic acid. To a stirred solution of diethyl 2-[[(5S,8S,10aR)-8-[[(1S)-3-carbamoyl-1-[[(4-methanesulfonylphenyl)methyl]carbamoyl]propyl]carbamoyl]-6-oxo-3-[(1r,4s)-4-[2-[1-(2,6-dioxopiperidin-3-yl)-3-methyl-2-oxo-1,3-benzodiazol-5-yl]ethyl]cyclohexanecarbonyl]-octahydropyrrolo[1,2-a][1,5]diazocin-5-yl]carbamoyl]-1H-indole-5-carbonylphosphonate (440.00 mg, 0.359 mmol) in DCM (12.00 mL) was added bromotrimethylsilane (879.59 mg, 5.75 mmol) at rt under nitrogen atmosphere. The resulting mixture was stirred at 40° C. overnight under nitrogen atmosphere. On completion, the reaction mixture was concentrated under reduced pressure. The residue was purified by reverse phase flash chromatography (Column: WelFlash™ C18-I, 330 g; EluentA: Water (plus 10 mmol/L FA); Eluent B: ACN; Gradient: 20%-40% B in 25 min; Flow rate: 80 mL/min; Detector: 220/254 nm; desired fractions were collected at 32% B) and concentrated under reduced pressure to afford the title compound (24.5 mg, 6% yield) as a white solid. ¹H NMR (400 MHz, DMSO-d6) δ 12.17-12.01 (m, 1H), 11.09 (s, 1H), 8.81 (s, 1H), 8.56 (t, J=6.3 Hz, 1H), 8.44 (d, J=6.2 Hz, 1H), 8.30 (dd, J=14.6, 7.5 Hz, 1H), 8.00-7.93 (m, 1H), 7.86 (d, J=8.2 Hz, 2H), 7.58-7.40 (m, 4H), 7.31-7.20 (m, 1H), 7.08-6.96 (m, 2H), 6.96-6.79 (m, 2H), 5.35 (dd, J=12.8, 5.4 Hz, 1H), 5.03-4.96 (m, 1H), 4.88-4.76 (m, 1H), 4.50-4.36 (m, 3H), 4.30-4.11 (m, 3H), 3.95 (d, J=13.5 Hz, 1H), 3.78-3.74 (m, 1H), 3.47 (d, J=3.1 Hz, 2H), 3.33 (s, 3H), 3.19 (s, 3H), 3.06-2.98 (m, 1H), 2.94-2.85 (m, 1H), 2.76-2.67 (m, 1H), 2.66-2.57 (m, 3H), 2.25-2.10 (m, 4H), 2.06-1.88 (m, 4H), 1.87-1.73 (m, 3H), 1.72-1.48 (m, 11H); LC/MS (ESI, m/z): [(M−H)]−=1167.1.

### Example 14: Synthesis of 2-[[(5S,8S,10aR)-8-[[(1S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl] methyl]cyclohexyl]acetyl]-octahydropyrrolo[1,2-a][1,5]diazocin-5-yl]carbamoyl]-1H-indole-5-carboxylic acid (I-75)

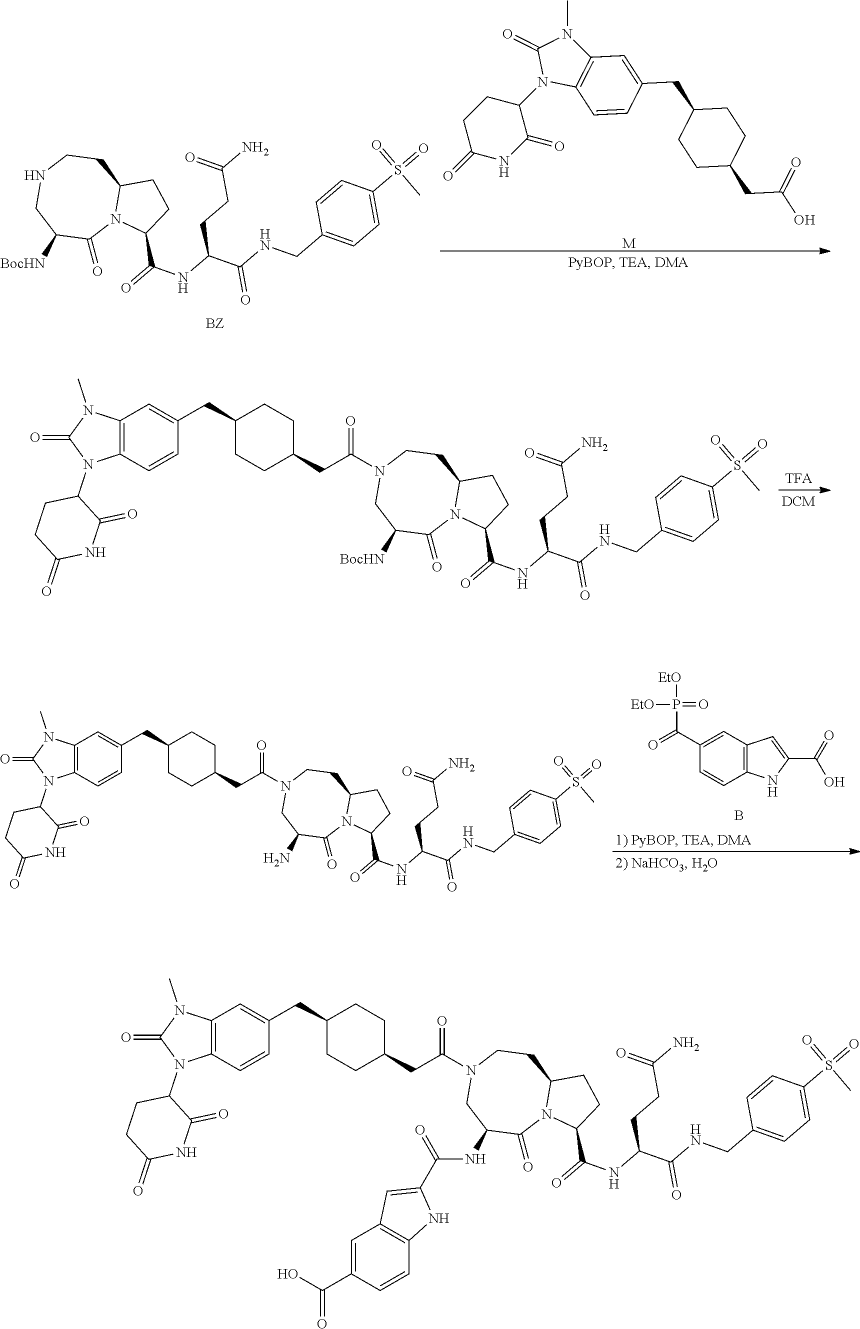

Step 1—Tert-butyl N-[(1S)-3-carbamoyl-1-[[3-(5-[[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamoyl]pentyl)-5-methylphenyl]carbamoyl]propyl]carbamate. To a stirred mixture of tert-butyl N-[(5S,8S,10aR)-8-[(3-carbamoyl-1-[[(4-methanesulfonylphenyl)methyl]carbamoyl]propyl)carbamoyl]-6-oxo-octahydro-1H-pyrrolo[1,2-a][1,5]diazocin-5-yl]carbamate (3.00 g, 4.82 mmol, Intermediate BZ) and [(1s,4s)-4-[[1-(2,6-dioxopiperidin-3-yl)-3-methyl-2-oxo-1,3-benzodiazol-5-yl]methyl]cyclohexyl]acetic acid (2.79 g, 6.74 mmol, Intermediate M) in DMA (30 mL) were added TEA (2.44 g, 24.1 mmol) and PyBOP (3.01 g, 5.78 mmol) in portions at rt under nitrogen atmosphere. After stirring for 1 h at rt under nitrogen atmosphere, the reaction mixture was concentrated under reduced pressure. The residue was purified by reverse phase flash chromatography (Column: Spherical C18, 20-40 um, 330 g; Mobile Phase A: Water (plus 5 mM NH4HCO3); Mobile Phase B: ACN; Flow rate: 80 mL/min; Gradient: 5%-5% B, 10 min, 30% B-50% B gradient in 20 min; Detector: UV 254/220 nm; the fractions containing the desired product were collected at 46% B_and concentrated under reduced pressure to afford the title compound (4.15 g, 85% yield) as a white solid. ¹H NMR (400 MHz, DMSO-d6) δ 10.76 (s, 1H), 8.58-8.52 (m, 1H), 8.32-8.27 (m, 1H), 7.87 (d, J=8.0 Hz, 2H), 7.59-7.51 (m, 2H), 7.23 (s, 1H), 7.05-6.94 (m, 2H), 6.89-6.70 (m, 2H), 6.55 (d, J=6.8 Hz, 1H), 5.34 (dd, J=12.7, 5.5 Hz, 1H), 4.53-4.32 (m, 4H), 4.28-4.22 (m, 1H), 4.18-4.09 (m, 2H), 3.85-3.70 (m, 1H), 3.49-3.42 (m, 1H), 3.34 (s, 3H), 3.22-3.15 (m, 6H), 3.12-2.99 (m, 1H), 2.98-2.85 (m, 1H), 2.77-2.56 (m, 6H), 2.42-2.35 (m, 2H), 2.26-2.10 (m, 2H), 2.09-1.89 (m, 2H), 1.87-1.55 (m, 4H), 1.45 (s, 9H), 1.43-1.38 (m, 6H), 1.18-0.91 (m, 2H); LC/MS (ESI, m/z): [(M+1)]⁺=1018.4.

Step 2—2-[[(5S,8S,10aR)-5-Amino-6-oxo-3-[2-[(1s,4s)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-N-[(4-methanesulfonylphenyl)methyl]pentanediamide. To a stirred solution oftert-butyl N-[(5S,8S,10aR)-8-[(3-carbamoyl-1-[[(4-methanesulfonylphenyl)methyl]carbamoyl]propyl)carbamoyl]-6-oxo-3-[2-[(1s,4s)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-5-yl]carbamate (4.15 g, 4.08 mmol) in DCM (30 mL) was added TFA (10 mL) in portions at rt under nitrogen atmosphere. The resulting mixture was stirred for 1 h at rt under nitrogen atmosphere. On completion, the reaction mixture was concentrated under reduced pressure. The residue was purified by reverse phase flash chromatography (Column: Spherical C18, 20-40 um, 330 g; Mobile Phase A: Water (plus 5 mM FA); Mobile Phase B: ACN; Flow rate: 80 mL/min; Gradient: 5%-5% B, 10 min, 20% B-40% B gradient in 20 min; Detector: UV 254/220 nm; the fractions containing the desired product were collected at 30% B) and concentrated under reduced pressure to afford the title compound (3.39 g, 90% yield) as a white solid. ¹H NMR (400 MHz, DMSO-d6) δ 11.09 (s, 1H), 8.58 (t, J=5.9 Hz, 1H), 8.36-8.29 (m, 1H), 8.24 (s, 2H), 7.87 (dd, J=8.4, 1.6 Hz, 2H), 7.56-7.48 (m, 2H), 7.26 (s, 1H), 7.03-6.97 (m, 2H), 6.87-6.76 (m, 2H), 5.34 (dd, J=12.7, 5.4 Hz, 1H), 4.49-4.36 (m, 3H), 4.28-4.20 (m, 1H), 4.19-4.12 (m, 2H), 3.86-3.81 (m, 1H), 3.72 (d, J=13.3 Hz, 1H), 3.20 (s, 3H), 2.93-2.84 (m, 1H), 2.73-2.63 (m, 5H), 2.46-2.27 (m, 1H), 2.19-2.12 (m, 4H), 2.08 (s, 3H), 2.04-1.91 (m, 1H), 1.89-1.80 (m, 8H), 1.47-1.41 (m, 8H), 1.38-1.32 (m, 2H); LC/MS (ESI, m/z): [(M+1)]+=918.5.

Step 3—2-[[(5S,8S,10aR)-8-[[(1S)-3-carbamoyl-1-[[(4-methanesulfonylphenyl)methyl]carbamoyl]propyl]carbamoyl]-6-oxo-3-[2-[(1s,4s)-4-[[1-(2,6-dioxopiperidin-3-yl)-3-methyl-2-oxo-1,3-benzodiazol-5-yl] methyl]cyclohexyl]acetyl]-octahydropyrrolo[1,2-a][1,5]diazocin-5-yl]carbamoyl]-1H-indole-5-carboxylic acid. To a stirred mixture of 2-[[(5S,8S,10aR)-5-amino-6-oxo-3-[2-[(1s,4s)-4-[[1-(2,6-dioxopiperidin-3-yl)-3-methyl-2-oxo-1,3-benzodiazol-5-yl]methyl]cyclohexyl]acetyl]-octahydropyrrolo[1,2-a][1,5]diazocin-8-yl]formamido]-N-[(4-methanesulfonylphenyl)methyl]pentanediamide (200.00 mg, 0.218 mmol) and 5-[(diethoxyphosphoryl)carbonyl]-1H-indole-2-carboxylic acid (70.86 mg, 0.218 mmol, Intermediate B) in DMA (8.00 mL) were added PyBOP (136.04 mg, 0.261 mmol) and TEA (0.09 mL, 0.654 mmol) in portions at rt under nitrogen atmosphere. The resulting mixture was stirred for 2 h at rt under nitrogen atmosphere. Next, sat. NaHCO₃(aq.) was added to the reaction mixture at rt and the mixture was stirred for 1 h at rt under nitrogen atmosphere. On completion, the residue was purified directly by reverse phase flash chromatography (Column: WelFlash™ C18-I, 20-40 m, 120 g; Eluent A: Water (plus 10 mmol/L FA); Eluent B: ACN; Gradient: 20%-40% B in 30 min; Flow rate: 60 mL/min; Detector: 220/254 nm; desired fractions were collected at 33% B) and concentrated under reduced pressure to afford the title compound (91.1 mg, 38% yield) as a white solid. ¹H NMR (400 MHz, DMSO-d6) δ 11.96 (s, 1H), 11.10 (s, 1H), 8.58 (s, 1H), 8.52-8.24 (m, 3H), 7.96-7.77 (m, 3H), 7.60-7.45 (m, 3H), 7.43-7.23 (m, 2H), 7.08-6.92 (m, 2H), 6.89-6.70 (m, 2H), 5.42-5.28 (m, 1H), 5.10-4.82 (m, 1H), 4.51-4.35 (m, 3H), 4.34-4.14 (m, 2H), 4.09-3.74 (m, 3H), 3.33 (s, 2H), 3.29 (s, 3H), 3.19 (s, 3H), 3.04-2.83 (m, 2H), 2.71-2.56 (m, 5H), 2.32-2.11 (m, 3H), 2.06-1.67 (m, 10H), 1.53-1.28 (m, 8H); LC/MS (ESI, m/z): [(M+H)]+=1105.4.

### Example 15: Synthesis of 2-{[(5S,8S,10aR)-3-Acetyl-8-{[(2S)-4-carbamoyl-1-(2-chloro-3-{5-[1-(2,6-dioxopiperidin-3-yl)-3-methyl-2-oxo-1,3-benzodiazol-5-yl]pentyl}phenoxy)butan-2-yl]carbamoyl}-6-oxo-octahydropyrrolo[1,2-a][1,5]diazocin-5-yl]carbamoyl}-1-benzothiophene-5-carbonylphosphonic acid (I-9I)

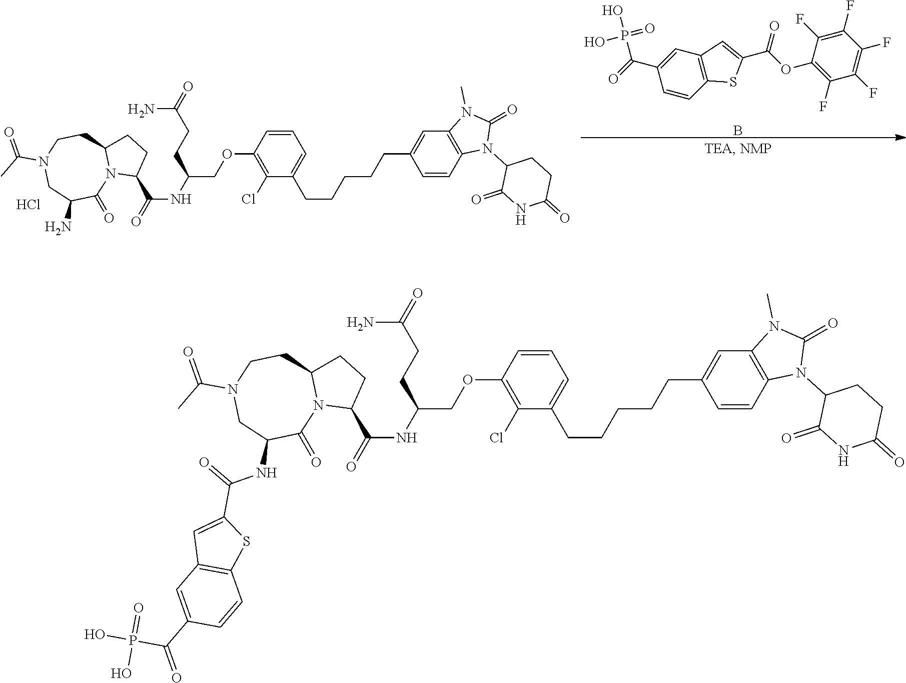

To a stirred solution of (4S)-4-{[(5S,8S,10aR)-3-acetyl-5-amino-6-oxo-octahydropyrrolo[1,2-a][1,5]diazocin-8-yl]formamido}-5-(2-chloro-3-{5-[1-(2,6-dioxopiperidin-3-yl)-3-methyl-2-oxo-1,3-benzodiazol-5-yl]pentyl}phenoxy)pentanamide hydrochloride (90 mg, 0.1 mmol, synthesized via Steps 1-2 of Example I-94) and 2-(2,3,4,5,6-pentafluorophenoxycarbonyl)-1-benzothiophene-5-carbonylphosphonic acid (47.45 mg, 0.11 mmol, Intermediate B) in DMA (1.00 mL) was added TEA (0.15 mL, 1.08 mmol) at 25° C. under nitrogen atmosphere. The resulting mixture was stirred for 2 h at rt under nitrogen atmosphere. On completion, the reaction mixture was concentrated under reduced pressure. The residue was purified by reverse flash chromatography (Column: Welflash™ C18-1, 20-40 um, 80 g; Eluent A: Water (plus 10 mmol/L FA); Eluent B: ACN; Gradient 10% to 40% B in 30 min; Flow rate: 50 mL/min; Detector: 220/254 nm; desired fractions were collected at 31% B) and concentrated under reduced pressure to afford the title compound (18.5 mg, 15% yield) as a white solid. ¹H NMR (400 MHz, DMSO-d6) δ 11.08 (s, 1H), 9.12-8.80 (m, 2H), 8.41-8.34 (m, 1H), 8.24-8.16 (m, 1H), 8.16-8.10 (m, 1H), 8.06-7.79 (m, 1H), 7.26-7.21 (m, 2H), 7.20-7.16 (m, 2H), 7.03-6.97 (m, 2H), 6.91-6.77 (m, 2H), 5.37-5.28 (m, 1H), 4.04-4.90 (m, 1H), 4.38-4.31 (m, 1H), 4.28-4.21 (m, 1H), 4.11-3.98 (m, 2H), 3.95-3.80 (m, 2H), 3.74-3.62 (m, 2H), 3.46-3.32 (m, 2H), 3.32 (s, 3H), 2.98-2.80 (m, 2H), 2.75-2.55 (m, 6H), 2.24-2.18 (m, 1H), 2.16-2.06 (m, 5H), 2.05-1.97 (m, 2H), 1.94-1.80 (m, 3H), 1.78-1.69 (m, 2H), 1.66-1.51 (m, 5H), 1.42-1.30 (m, 2H); LC/MS (ESI, m/z): [(M+H)]+=1089.5.

### Example 16: Synthesis of (2-{[(3S,6S,10aS)-3-{[(2S)-4-carbamoyl-1-(2-chloro-3-{5-[1-(2,6-dioxopiperidin-3-yl)-3-methyl-2-oxo-1,3-benzodiazol-5-yl]pentyl}phenoxy)butan-2-yl]carbamoyl}-5-oxo-octahydro-1H-pyrrolo[1,2-a]azocin-6-yl]carbamoyl}-1-benzothiophen-5-yl)difluoromethylphosphonic acid (I-93)

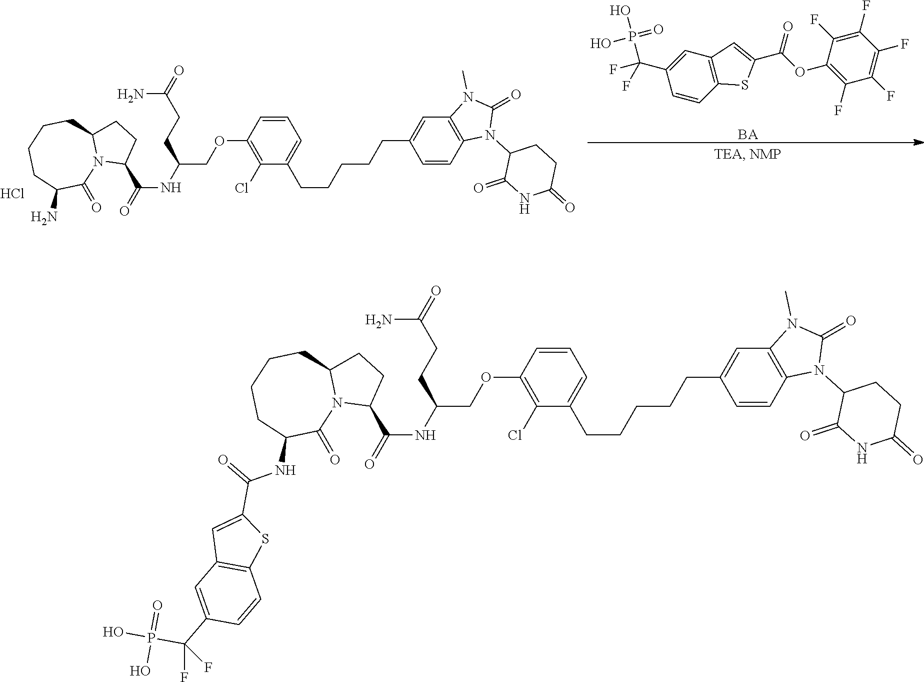 [SMILES: Cl ; Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](N)C(=O)N54)c3Cl)cc21 ; O=C(Oc1c(F)c(F)c(F)c(F)c1F)c1cc2cc(C(F)(F)P(=O)(O)O)ccc2s1 ; "BA" ; Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(F)(F)P(=O)(O)O)ccc7s6)C(=O)N54)c3Cl)cc21]

To a stirred solution of (4S)-4-{[(3S,6S,10aS)-6-amino-5-oxo-octahydro-H-pyrrolo[11,2-a]azocin-3-yl]formamido}-5-(2-chloro-3-{5-[1-(2,6-dioxopiperidin-3-yl)-3-methyl-2-oxo-1,3-benzodiazol-5-yl]pentyl}phenoxy)pentanamide hydrochloride (100 mg, 0.123 mmol, synthesized via Steps 1-2 of Intermediate 1-90) in NMP (2 mL) were added TEA (0.34 mL, 2.46 mmol) and difluoro[2-(2,3,4,5,6-pentafluorophenoxycarbonyl)-1-benzothiophen-5-yl]methylphosphonic acid (69.84 mg, 0.148 mmol, Intermediate BA) in turns at rt under air atmosphere. The resulting mixture was stirred for 1 h at rt under air atmosphere. On completion, the residue was purified by directly reverse phase flash chromatography (Column: WelFlash™ C18-I, 20-40 um, 120 g; Eluent A: Water (plus 10 mmol/L FA); Eluent B: ACN; Gradient: 5%-35% B in 25 min; Flow rate: 60 mL/min; Detector: 220/254 nm; desired fractions were collected at 32% B) and concentrated under reduced pressure to afford the title compound (17.4 mg, 12% yield) as a white solid. ¹H NMR (400 MHz, DMSO-d6) δ 11.10-11.06 (m, 1H), 8.95-8.91 (m, 1H), 8.32 (s, 1H), 8.13-8.09 (m, 1H), 8.06 (s, 1H), 8.03-7.99 (m, 1H), 7.59-7.55 (m, 1H), 7.21-7.14 (m, 2H), 7.05-6.95 (m, 3H), 6.94-6.81 (m, 2H), 6.77-6.73 (m, 1H), 5.36-5.32 (m, 1H), 4.97-4.91 (m, 1H), 4.33-4.29 (m, 2H), 4.13-4.09 (m, 1H), 4.03-3.99 (m, 1H), 3.93-3.89 (m, 1H), 3.32 (s, 3H), 2.95-2.96 (m, 1H), 2.78-2.67 (m, 3H), 2.67-2.57 (m, 3H), 2.22-2.16 (m, 2H), 2.11-1.94 (m, 4H), 1.89-1.78 (m, 7H), 1.70-1.52 (m, 10H), 1.42-1.30 (m, 2H); LC/MS (ESI, m/z): [(M+H)]+=1068.5.

### Example 17: Synthesis of (2-{1[(5S,8S,10aR)-8-{1[(2S)-4-carbamoyl-1-(2-chloro-3-{5-[1-(2, 6-dioxopiperidin-3-yl)-3-methyl-2-oxo-1, 3-benzodiazol-5-yl] pentyl} phenoxy) butan-2-yl]carbamoyl}-3-(methoxycarbonyl)-6-oxo-octahydropyrrolo [1,2-a][1,5] diazocin-5-yl] carbamoyl}-1-benzothiophen-5-yl) difluoromethylphosphonic acid (I-95)

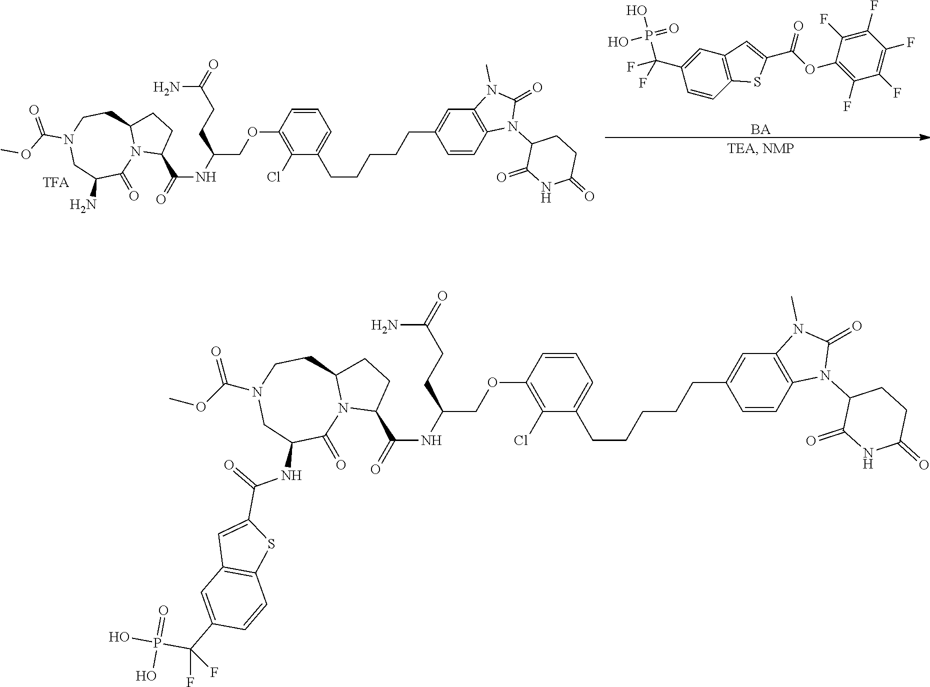 [SMILES: COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](N)C1 ; O=C(Oc1c(F)c(F)c(F)c(F)c1F)c1cc2cc(C(F)(F)P(=O)(O)O)ccc2s1 ; "BA" ; COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(F)(F)P(=O)(O)O)ccc3s2)C1 ; "TFA"]

To a stirred solution of methyl (5S,8S,10aR)-5-amino-8-{[(2S)-4-carbamoyl-1-(2-chloro-3-{5-[1-(2,6-dioxopiperidin-3-yl)-3-methyl-2-oxo-1,3-benzodiazol-5-yl] pentyl} phenoxy) butan-2-yl]carbamoyl}-6-oxo-octahydropyrrolo [1,2-a][1,5] diazocine-3-carboxylate trifluoroacetate (120 mg, 0.13 mmol, synthesized via Steps 1-2 of Intermediate I-92) and difluoro [2-(2,3,4,5,6-pentafluorophenoxycarbonyl)-1-benzothiophen-5-yl] methylphosphonic acid (121.68 mg, 0.256 mmol, Intermediate BA) in NMP (3 mL) was added TEA (0.18 mL, 1.28 mmol) dropwise at rt under nitrogen atmosphere. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 m, 120 g; Eluent A: Water (plus 10 mmol/L FA); Eluent B: ACN; Gradient: 15%-45% B in 35 min; Flow rate: 60 mL/min; Detector: 220/254 nm; desired fractions were collected at 34% B) and concentrated under reduced pressure to afford the title compound (17.4 mg, 12% yield) as a white solid. ¹H NMR (400 MHz, DMSO-d6) δ 11.09 (s, 1H), 9.10-8.97 (m, 1H), 8.34-8.29 (m, 1H), 8.15-8.08 (m, 2H), 8.04-7.99 (m, 1H), 7.62-7.58 (m, 1H), 7.25-7.11 (m, 2H), 7.04-6.95 (m, 3H), 6.91-6.83 (m, 2H), 6.78-6.71 (m, 1H), 5.37-5.30 (m, 1H), 4.95-4.87 (m, 1H), 4.36-4.22 (m, 3H), 4.10-3.96 (m, 3H), 3.95-3.86 (m, 2H), 3.84-3.74 (m, 2H), 3.69-3.61 (m, 4H), 3.57-3.44 (m, 3H), 3.32 (s, 3H), 2.97-2.83 (m, 1H), 2.73-2.58 (m, 5H), 2.21-2.10 (m, 3H), 2.05-1.96 (m, 2H), 1.92-1.80 (m, 3H), 1.76-1.68 (m, 2H), 1.65-1.54 (m, 4H), 1.41-1.32 (m, 1H); LC/MS (ESI, m/z): [(M+H)]+=1127.6.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-a: 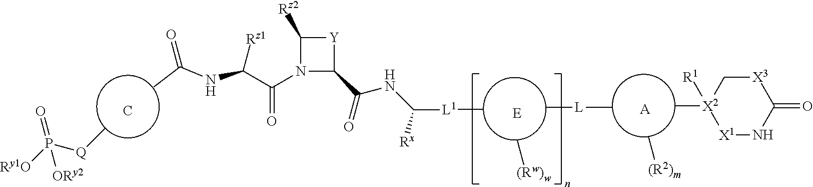
or a pharmaceutically acceptable salt thereof, wherein:
X¹ is a bivalent moiety selected from a covalent bond, —CR₂—, —C(O)—, —C(S)—, —CR(CF₃)—, —P(O)OR—, —P(O)R—, —P(O)NR₂—, —S(O)—, —S(O)₂—, or 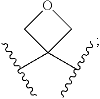 [SMILES: *C1(*)COC1]
X² is a carbon atom or silicon atom;
X³ is a bivalent moiety selected from —CR₂—, —NR—, —O—, —S—, or —SiR₂—;
R¹ is hydrogen, halogen, —CN, —OR, —SR, —S(O)R, —S(O)₂R, —NR₂, —P(O)(OR)₂, —P(O)NR₂OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
each R² is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bicyclic or tricyclic ring selected from 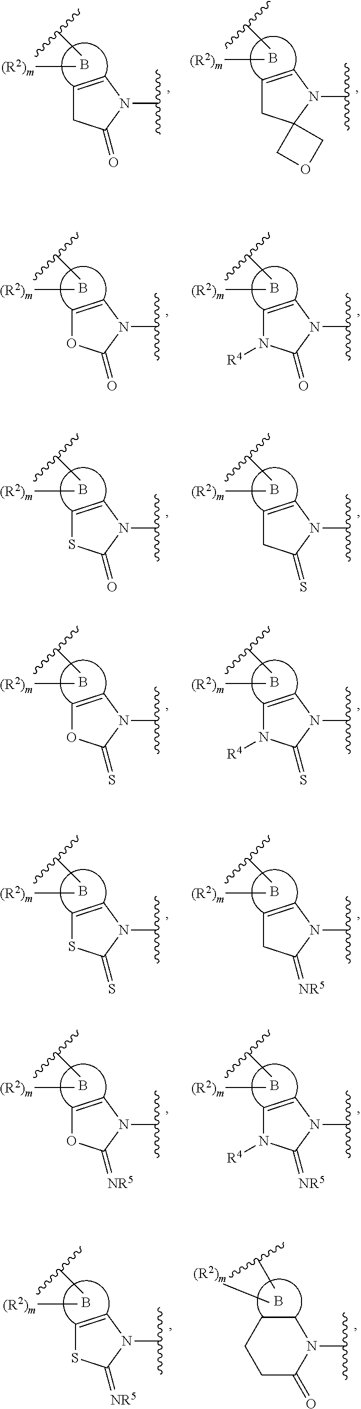 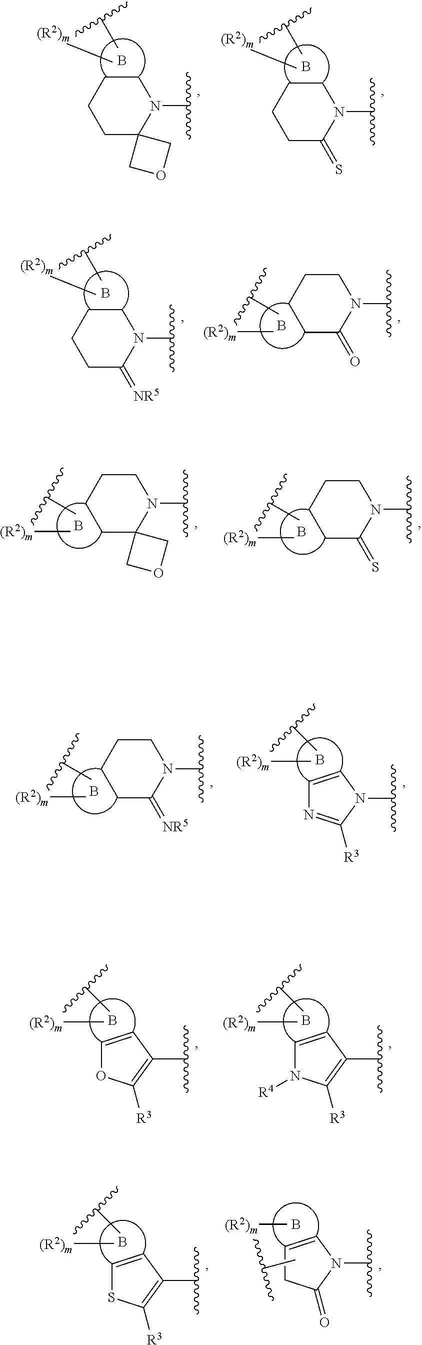 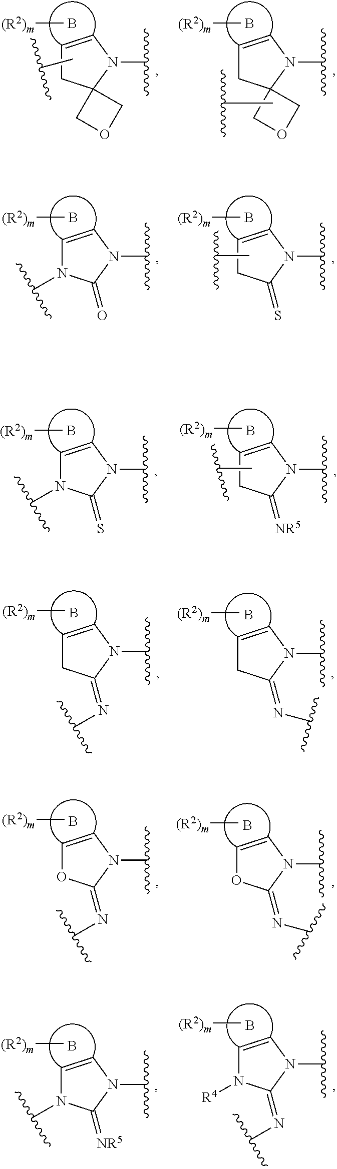 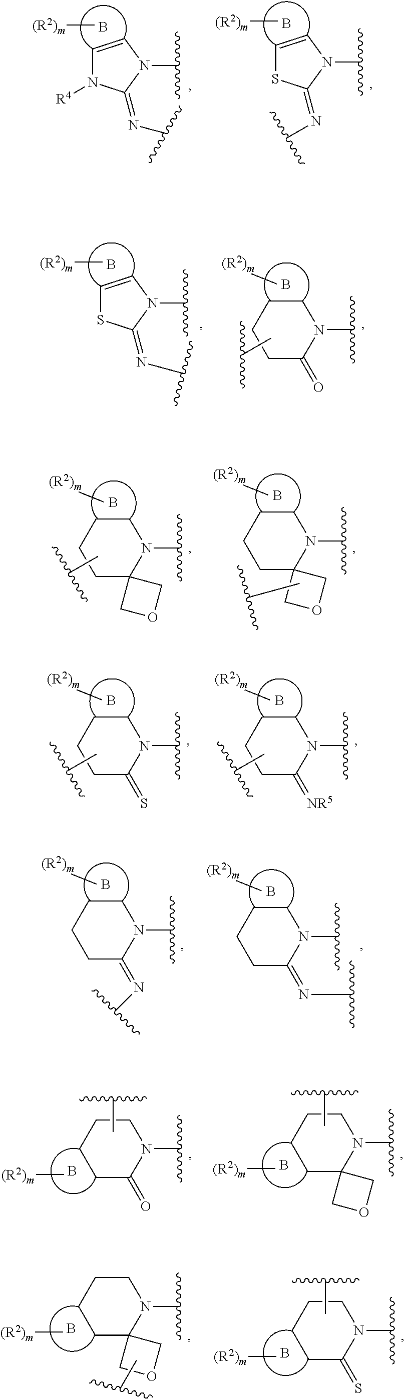 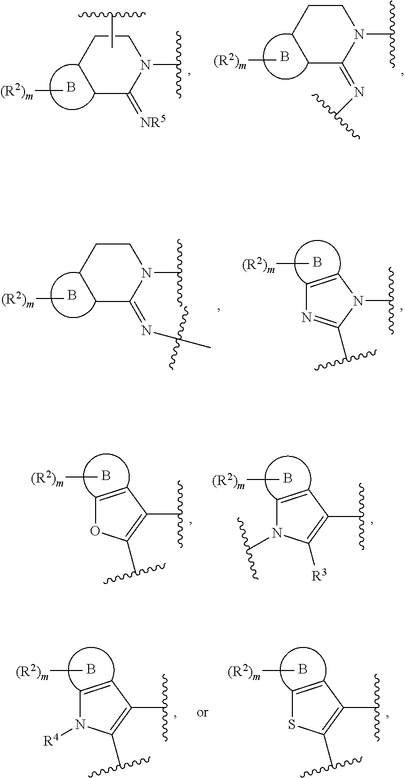
wherein
Ring B is a fused ring selected from benzo, 5-6 membered heteroaryl containing 1-4 heteroatoms independently selected from nitrogen, oxygen, or sulfur, and a 5 to 7-membered saturated or partially unsaturated carbocyclyl or heterocyclyl with 1-3 heteroatoms independently selected from nitrogen, oxygen, or sulfur;
R³ is selected from hydrogen, halogen, —OR, —NR₂, or —SR;
each R⁴ is independently hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
R⁵ is hydrogen, C₁₋₄ aliphatic, or —CN;
m is 0, 1, 2, 3 or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
Y is an optionally substituted —(CH₂)_{y}—, wherein:
y is 1, 2, or 3;
R^{x} is hydrogen, R^{A}, —(CR₂)₁₋₃OCONR₂, or —(CR₂)₁₋₃CONR₂;
R^{y1} and R^{y2} are each independently hydrogen, R^{A}, —CH₂CO₂R, or —CH₂OCO₂R;
R^{z1} and R^{z2} are each independently hydrogen or R^{A}, or:
R^{z1} and R^{z2} are cyclically linked to form an optionally substituted fused 5-8 membered heterocyclic ring;
Ring C is an optionally substituted is a bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Ring E is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

2. The compound of claim 1, wherein said compound is any one of the following formulae: 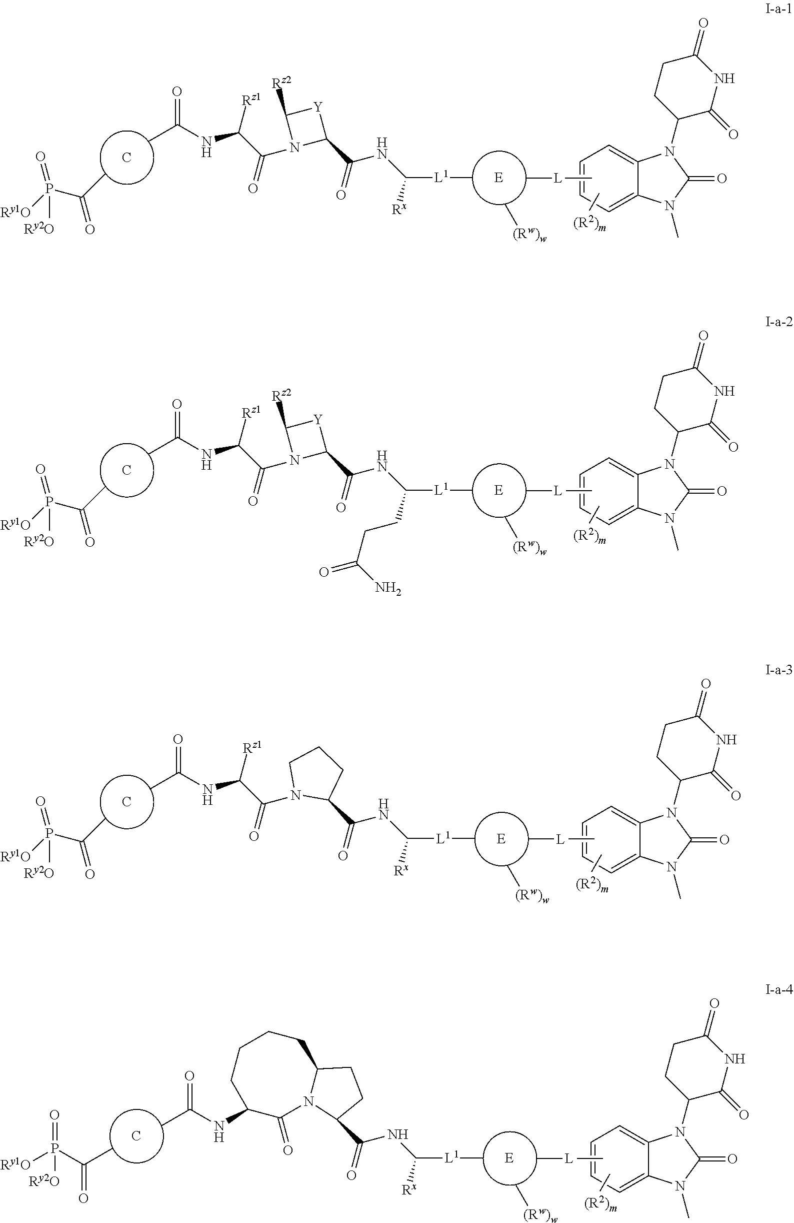 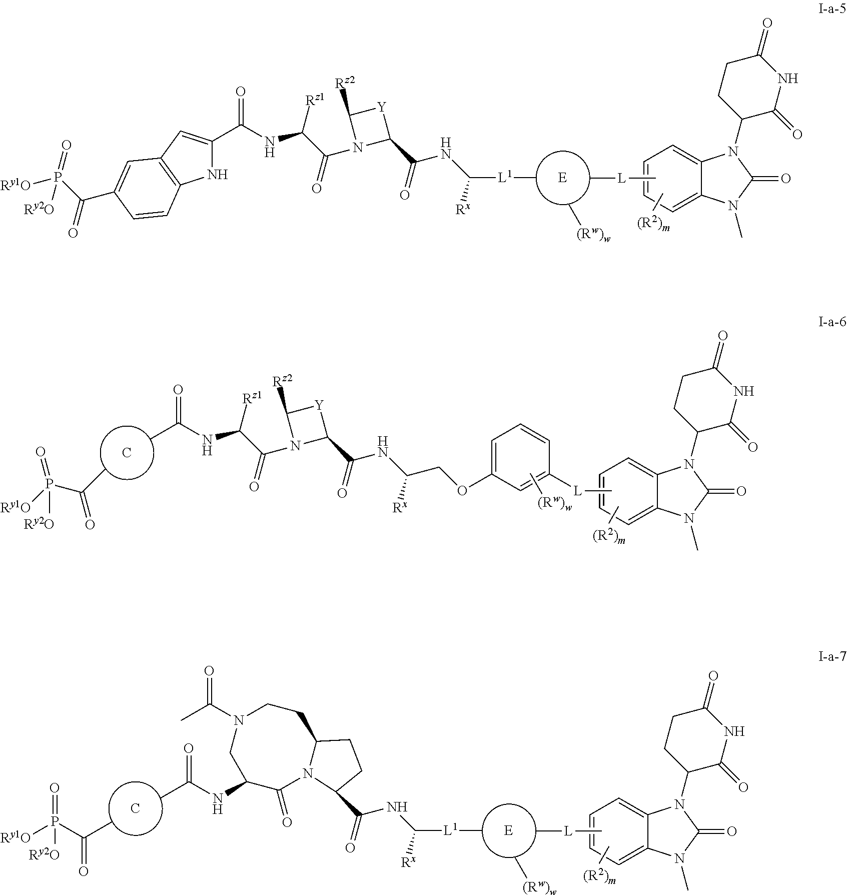
or a pharmaceutically acceptable salt thereof.

3. A compound of formula I-b or I-f: 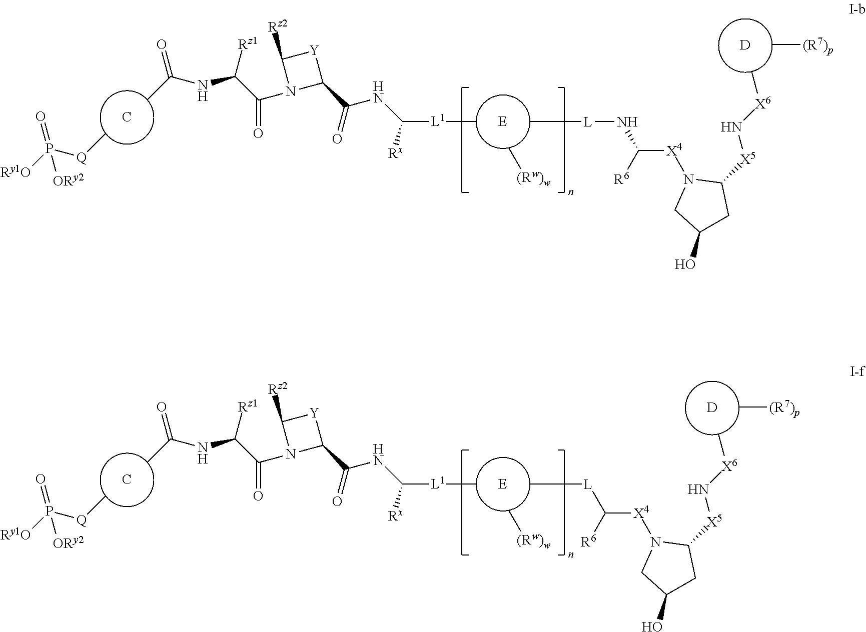
or a pharmaceutically acceptable salt thereof, wherein:
X⁴, X⁵, and X⁶ are each independently a bivalent moiety selected from a covalent bond, —CR₂—, —C(O)—, —C(S)—O—, —S(O)—, —S(O)₂—, 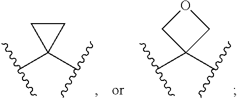 [SMILES: *C1(*)CC1 ; *C1(*)COC1]
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
R⁶ is hydrogen or R^{A};
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring D is selected from phenyl, a 4-11 membered saturated or partially unsaturated monocyclic, bicyclic, bridged bicyclic, or spirocyclic carbocyclic or heterocyclic ring having 1-3 heteroatoms independently selected from nitrogen, oxygen, and sulfur, and a 5-6 membered heteroaryl with 1-4 heteroatoms independently selected from nitrogen, oxygen or sulfur;
R⁷ is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)NR₂, or —NRS(O)₂R;
p is 0, 1, 2, 3, or 4;
L is a covalent bond or a bivalent, saturated or partially unsaturated, straight or branched C₁₋₂₀ hydrocarbon chain, wherein 0-6 methylene units of L are independently replaced by -Cy-, —O—, —NR—, —CRF—, —CF₂—, —C(O)—, —S—, —S(O)—, —S(O)₂—, —SiR₂—, —Si(OH)R—, —Si(OH)₂—, —P(O)OR—, —P(O)R—, or —P(O)NR₂—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
L¹ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—;
Q is a bivalent moiety selected from —O—, —CR₂—, —CF₂—, —CFR—, —C(O)—, —OCR₂—, and —C(S)—;
Y is an optionally substituted —(CH₂)_{y}—, wherein:
y is 1, 2, or 3;
R^{x} is hydrogen, R^{A}, —(CR₂)₁₋₃OCONR₂, or —(CR₂)₁₋₃CONR₂;
R^{yl} and R^{y2} are each independently hydrogen, R^{A}, —CH₂CO₂R, or —CH₂OCO₂R;
R^{z1} and R^{z2} are each independently hydrogen or R^{A}, or:
R^{z1} and R^{z2} are cyclically linked to form an optionally substituted fused 5-8 membered heterocyclic ring;
Ring C is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, and sulfur;
Ring E is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur;
R^{w} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)NR₂, —NRS(O)₂R, —NP(O)R₂, —NRP(O)(OR)₂, —NRP(O)(OR)NR₂, —NRP(O)(NR₂)₂, or —NRS(O)₂R;
w is 0, 1, 2, 3, or 4; and
n is 0 or 1.

4. The compound of claim 3, wherein said compound is any one of the following formulae: 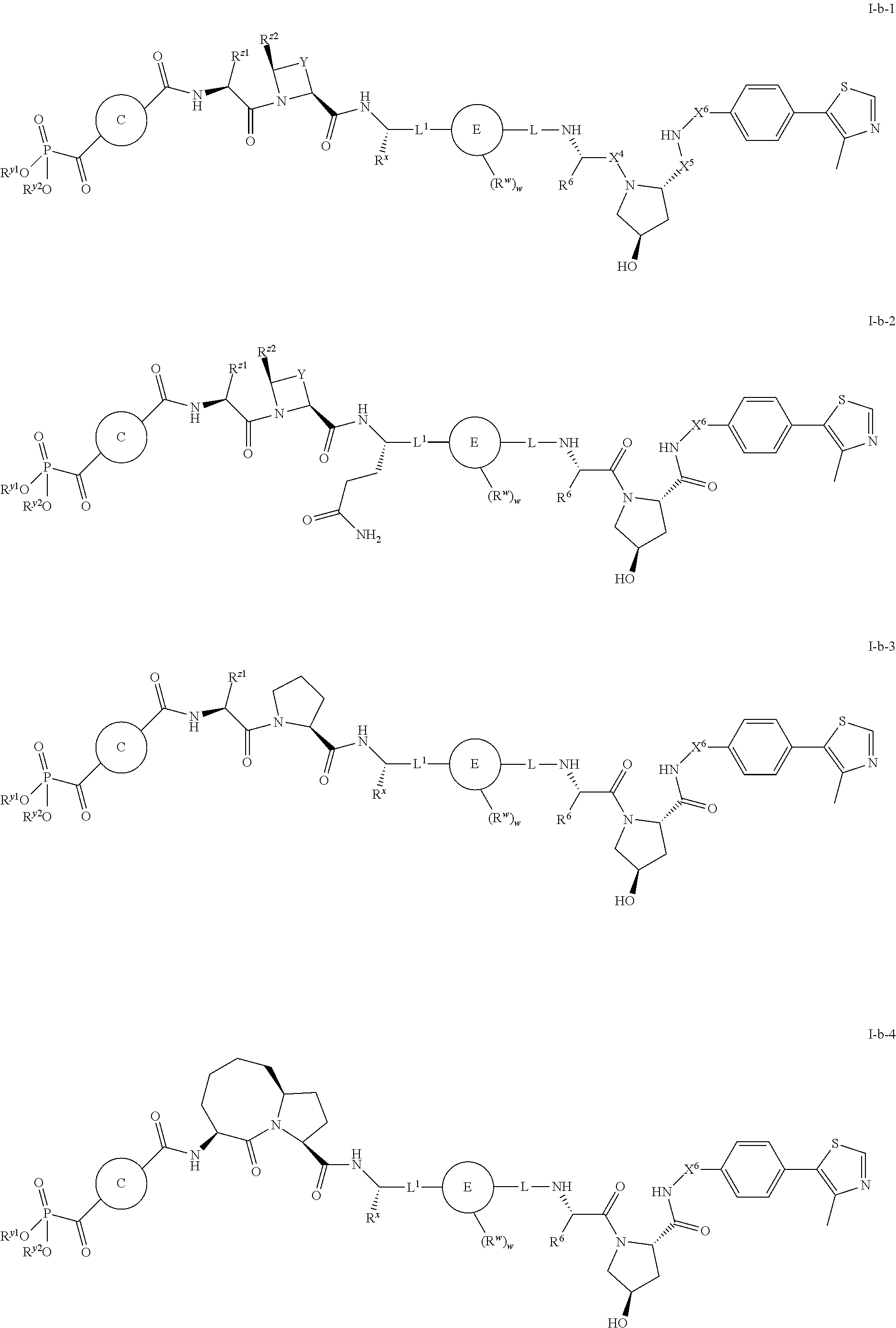 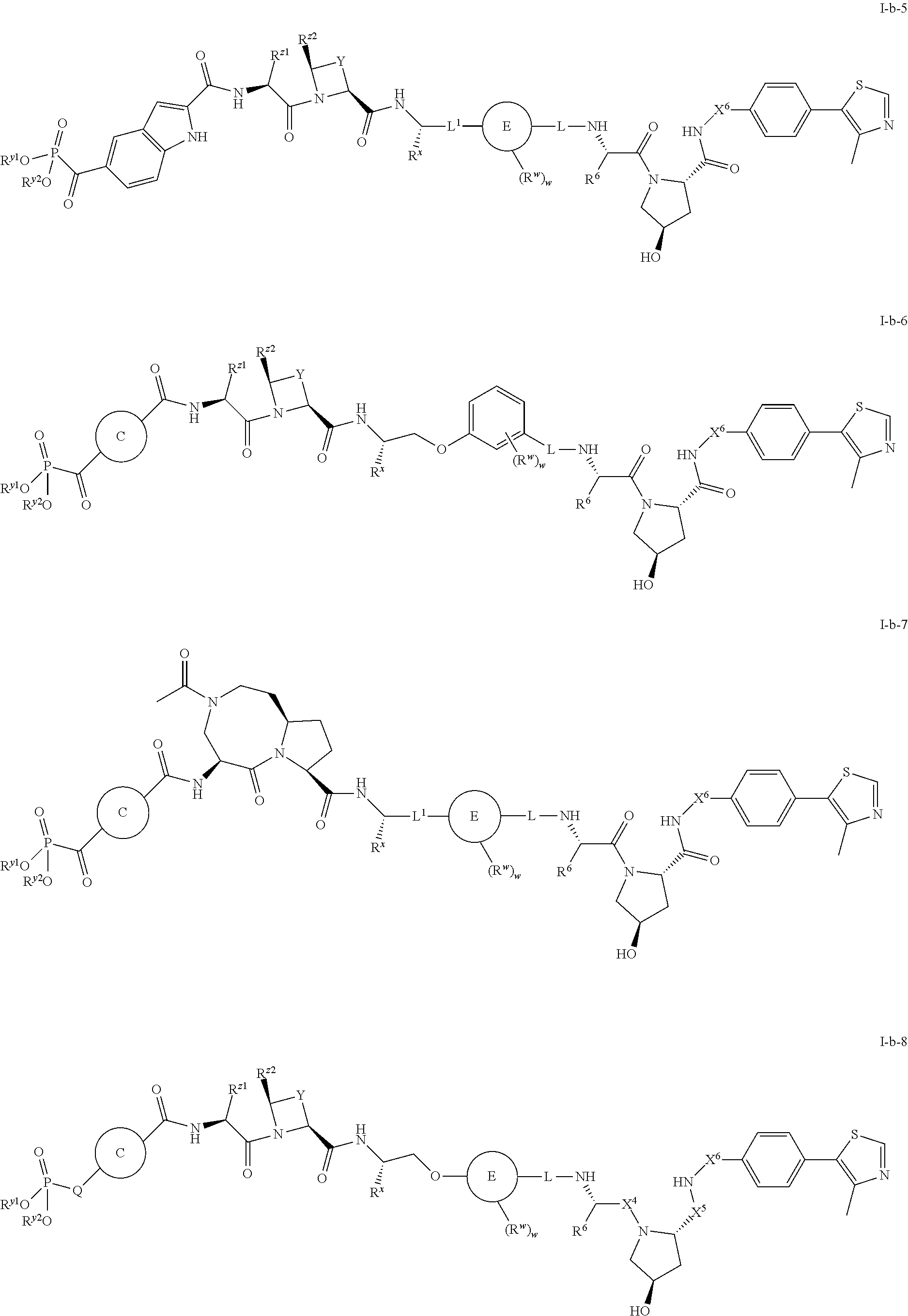 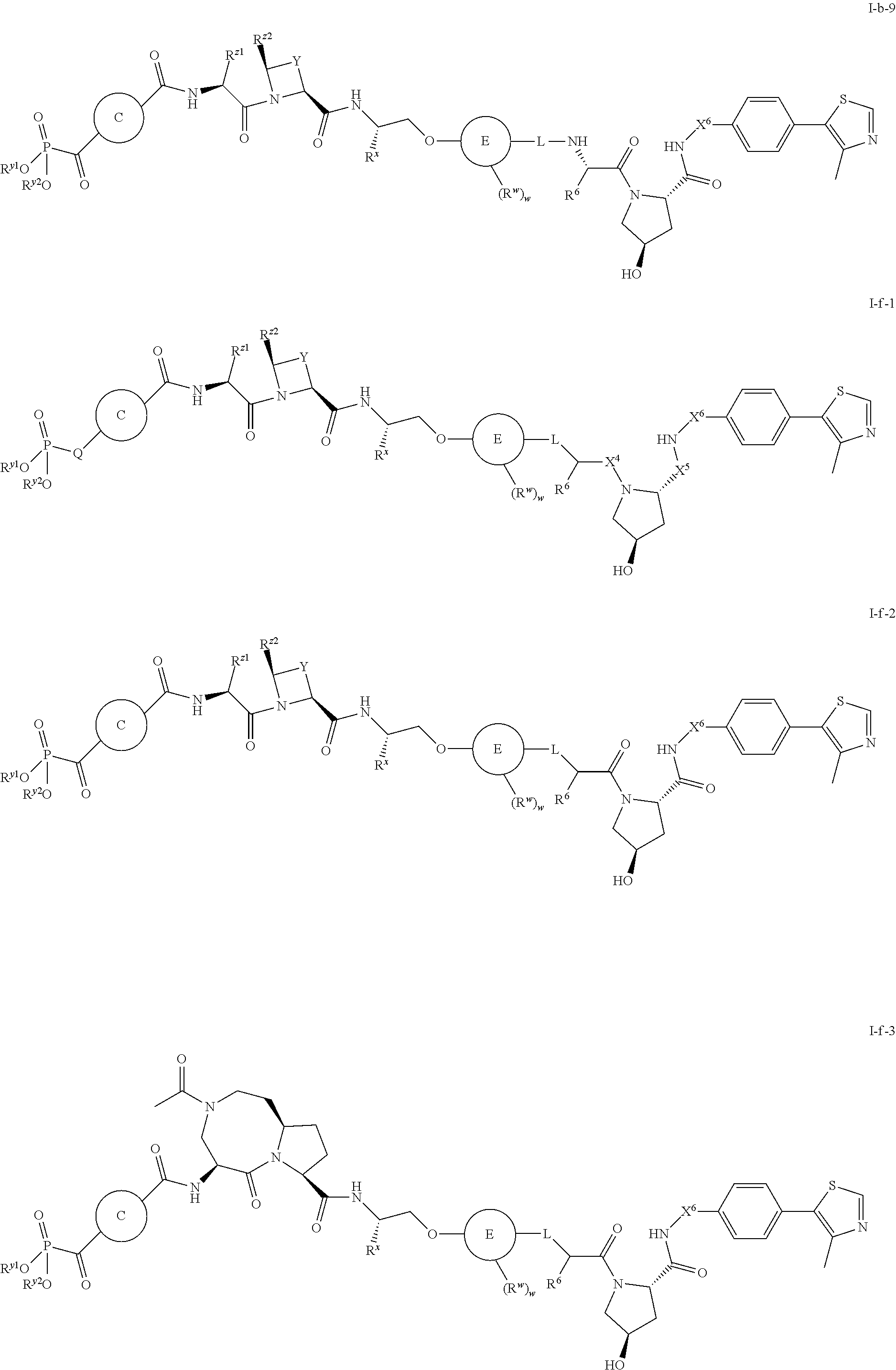
or a pharmaceutically acceptable salt thereof.

5. The compound of claim 3, wherein R^{z1} is selected from hydrogen, methyl, —CH₂CH₂OH, ethyl, isopropyl, neopropyl, tert-butyl, cyclopropyl, cyclobutyl, cyclopentyl, and cyclohexyl.

6. The compound of claim 3, wherein R⁶ is selected from ethyl, isopropyl, neopropyl, tert-butyl, cyclopropyl, cyclobutyl, cyclopentyl, and cyclohexyl.

7. The compound of claim 3, wherein X⁶ is selected from

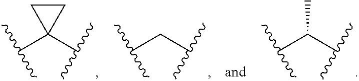

8. The compound of claim 3, wherein Ring E is selected from

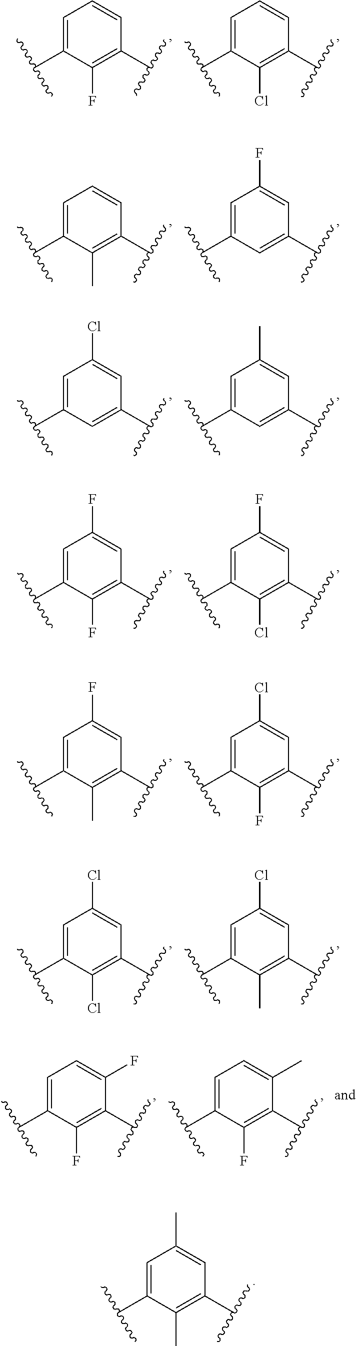

9. The compound of claim 3, wherein L is selected from —CH₂—, —CH₂CH₂—, —CH₂NH—, —CH₂CH₂CH₂—, —CH₂CH₂CH₂CH₂—,

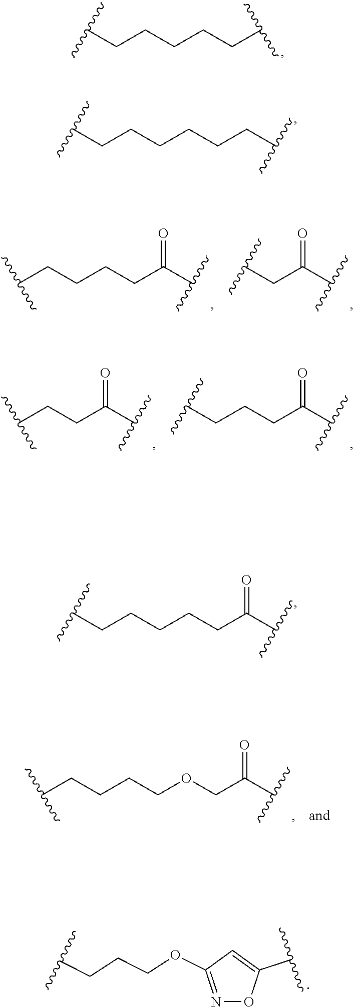

10. The compound of claim 1 or claim 3, wherein said compound is selected from 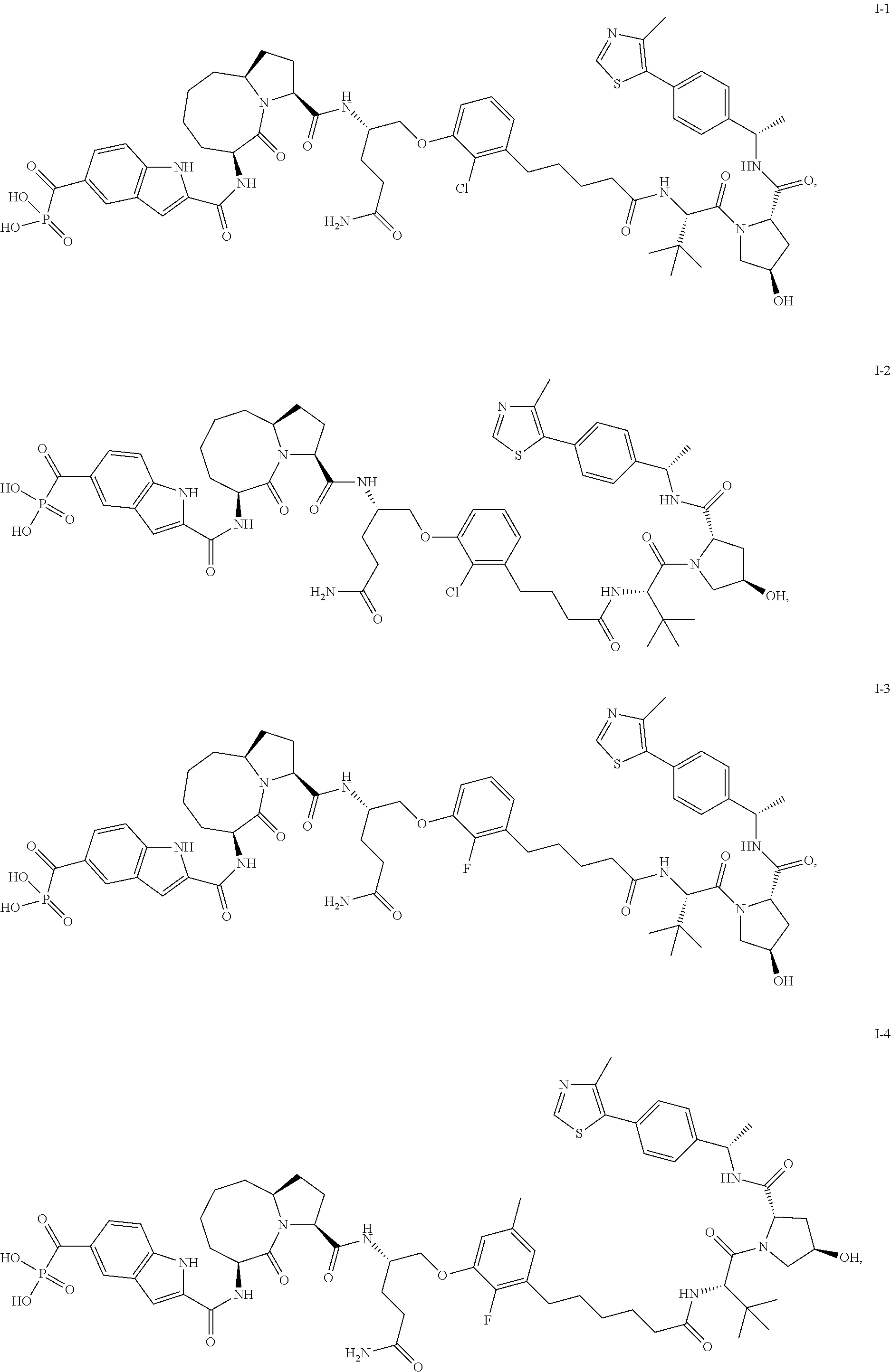 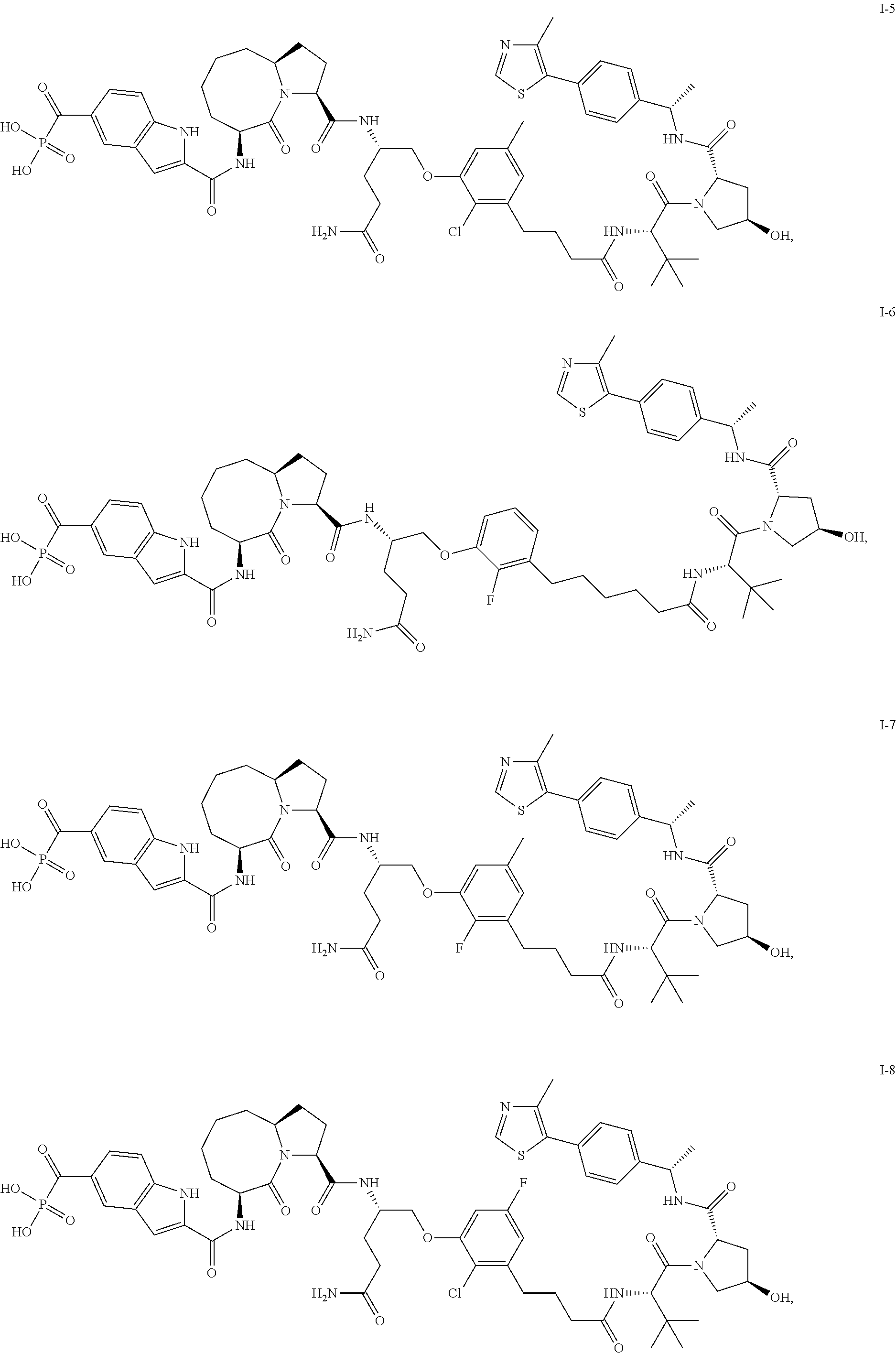 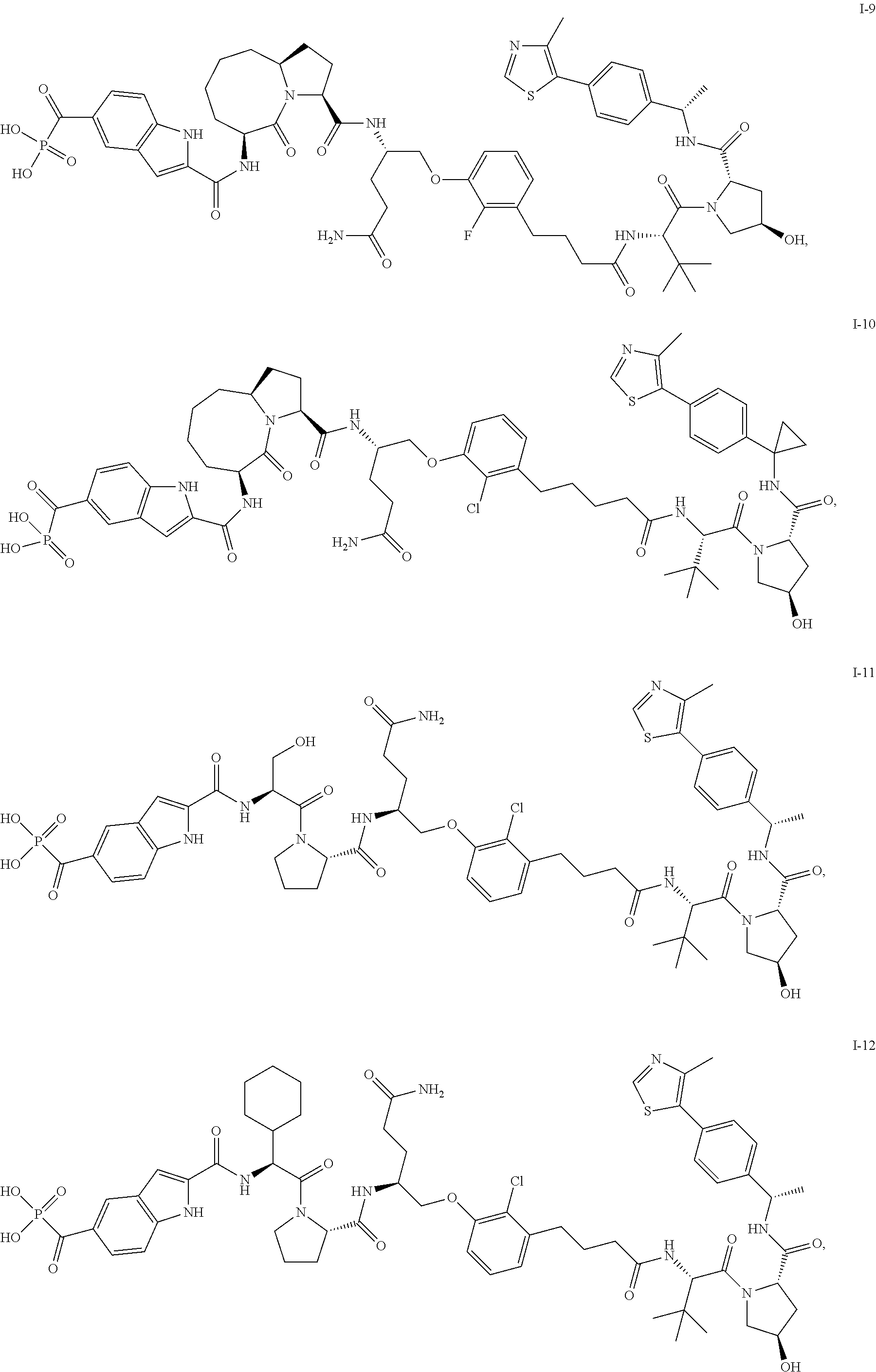 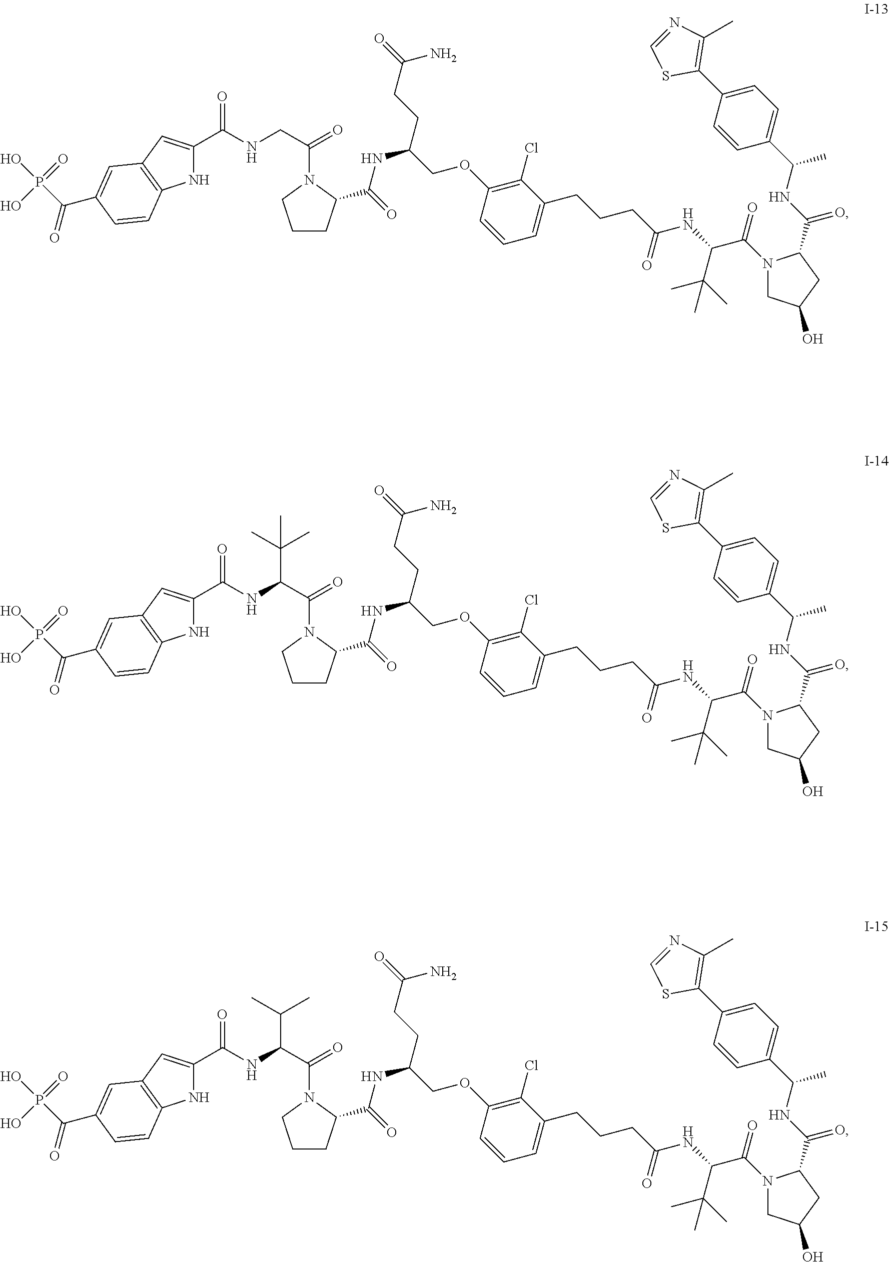 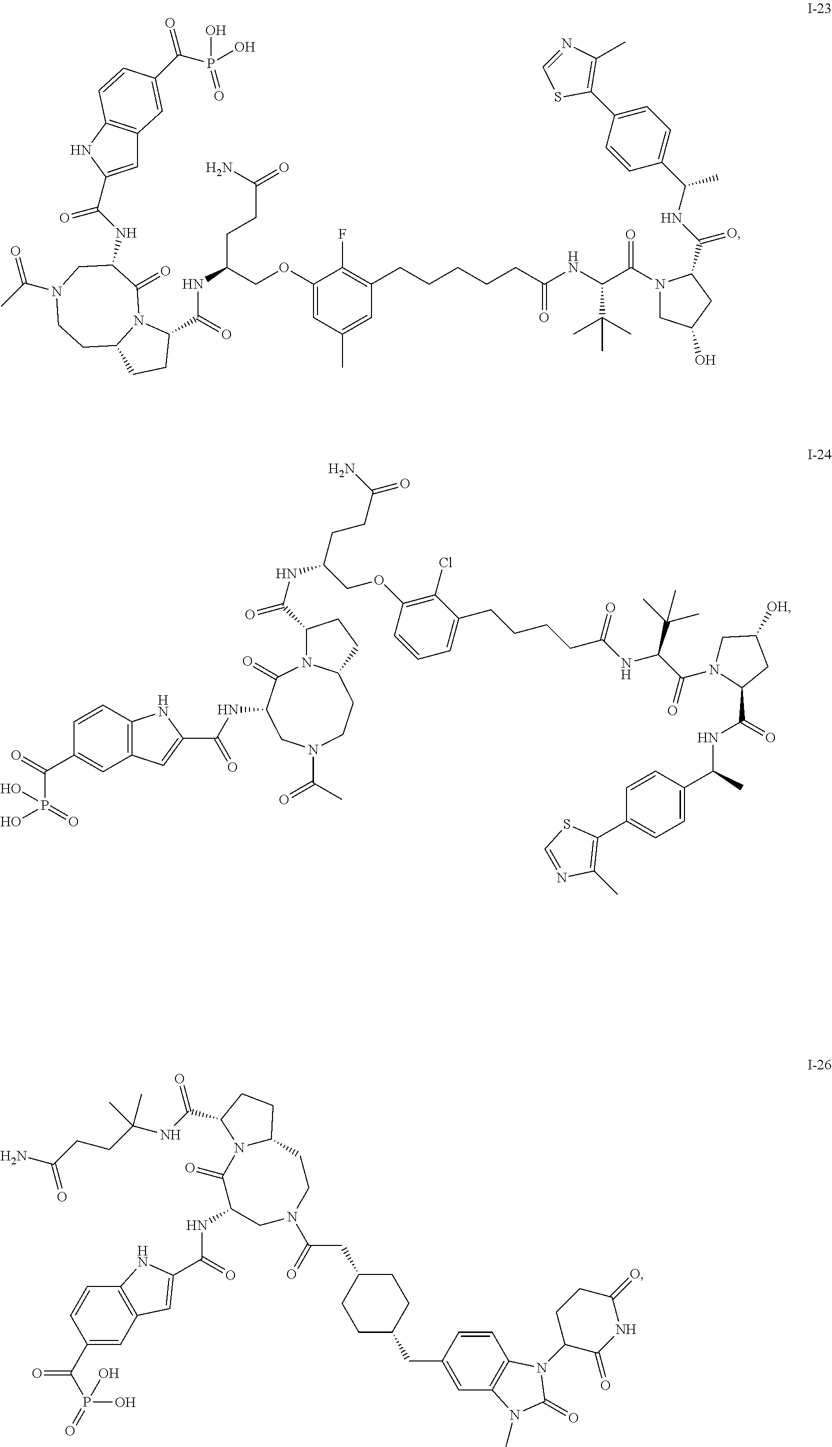 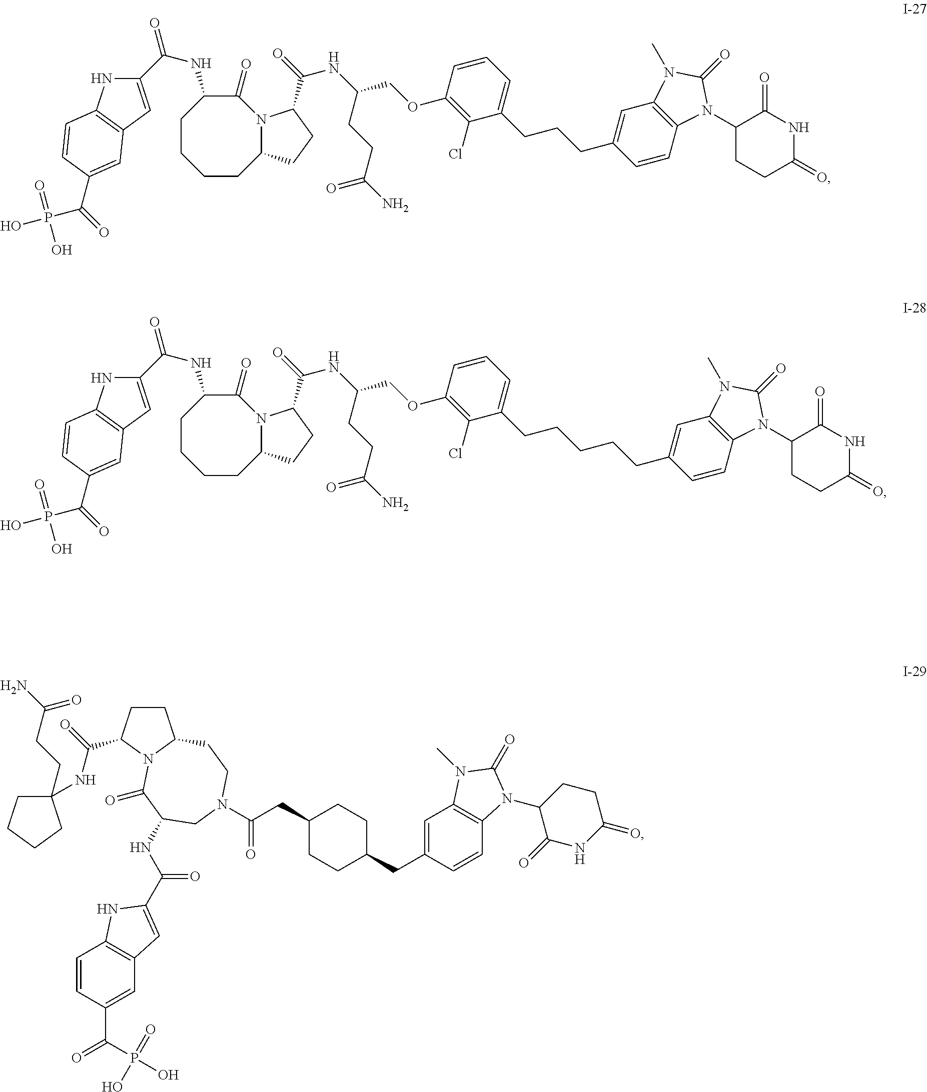 [SMILES: Cn1c(=O)n(C2CCC(=O)NC2=O)c2ccc(CCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21 ; Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21 ; Cn1c(=O)n(C2CCC(=O)NC2=O)c2ccc(C[C@H]3CC[C@@H](CC(=O)N4CC[C@H]5CC[C@@H](C(=O)NC6(CCC(N)=O)CCCC6)N5C(=O)[C@@H](NC(=O)c5cc6cc(C(=O)P(=O)(O)O)ccc6[nH]5)C4)CC3)cc21] 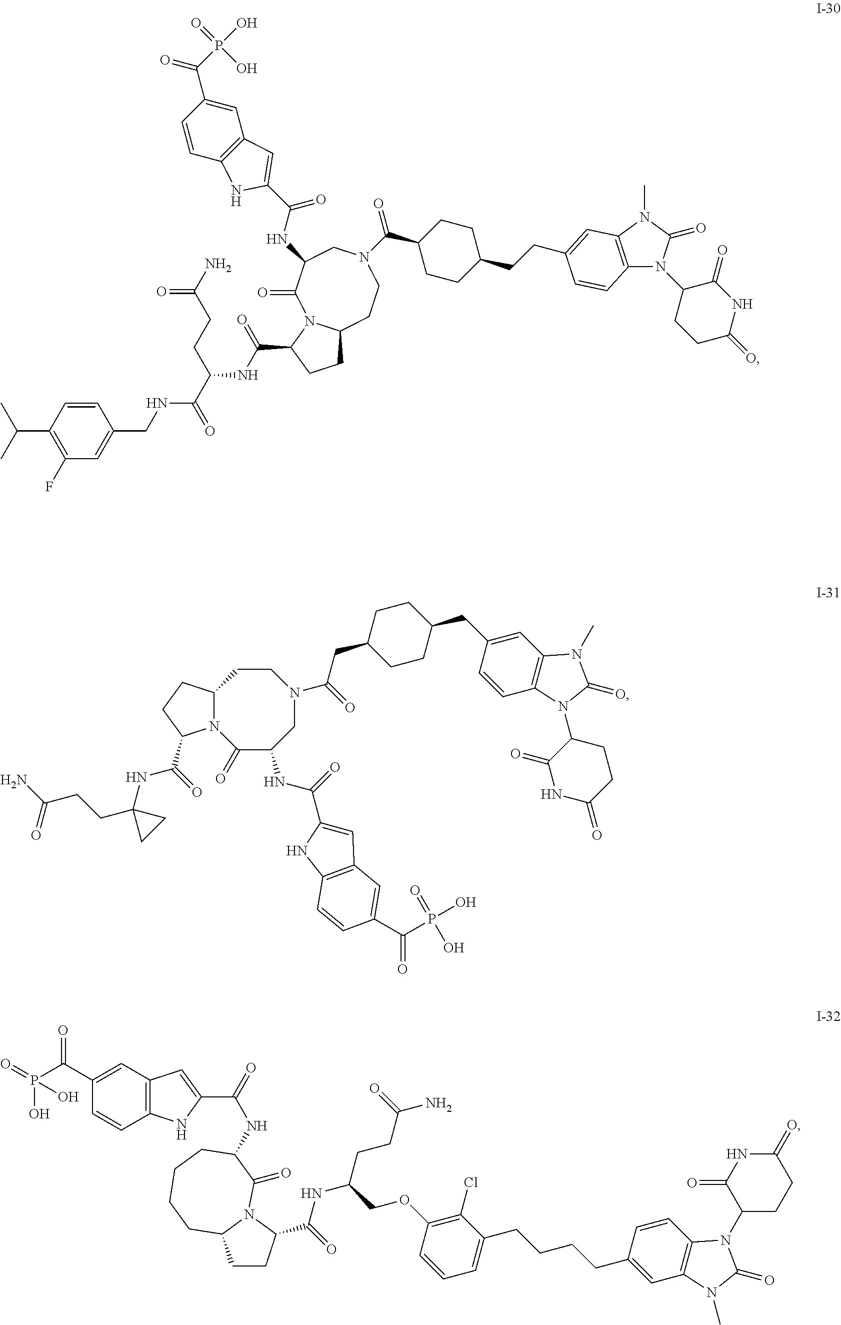 [SMILES: CC(C)c1ccc(CNC(=O)[C@H](CCC(N)=O)NC(=O)[C@@H]2CC[C@@H]3CCN(C(=O)[C@H]4CC[C@@H](CCc5ccc6c(c5)n(C)c(=O)n6C5CCC(=O)NC5=O)CC4)C[C@H](NC(=O)c4cc5cc(C(=O)P(=O)(O)O)ccc5[nH]4)C(=O)N32)cc1F ; Cn1c(=O)n(C2CCC(=O)NC2=O)c2ccc(C[C@H]3CC[C@@H](CC(=O)N4CC[C@H]5CC[C@@H](C(=O)NC6(CCC(N)=O)CC6)N5C(=O)[C@@H](NC(=O)c5cc6cc(C(=O)P(=O)(O)O)ccc6[nH]5)C4)CC3)cc21 ; Cn1c(=O)n(C2CCC(=O)NC2=O)c2ccc(CCCCc3cccc(OC[C@H](CCC(N)=O)NC(=O)[C@@H]4CC[C@@H]5CCCC[C@H](NC(=O)c6cc7cc(C(=O)P(=O)(O)O)ccc7[nH]6)C(=O)N54)c3Cl)cc21] 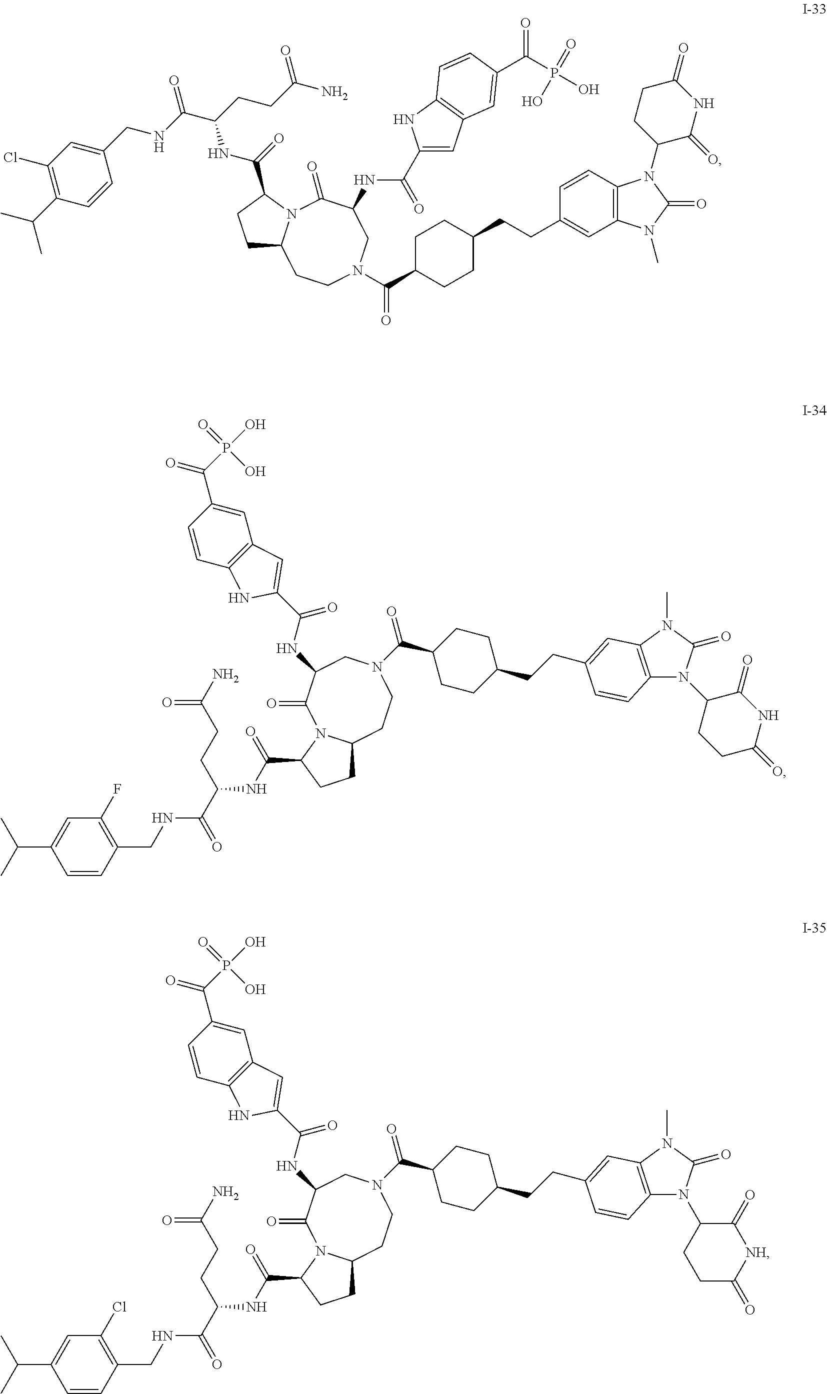 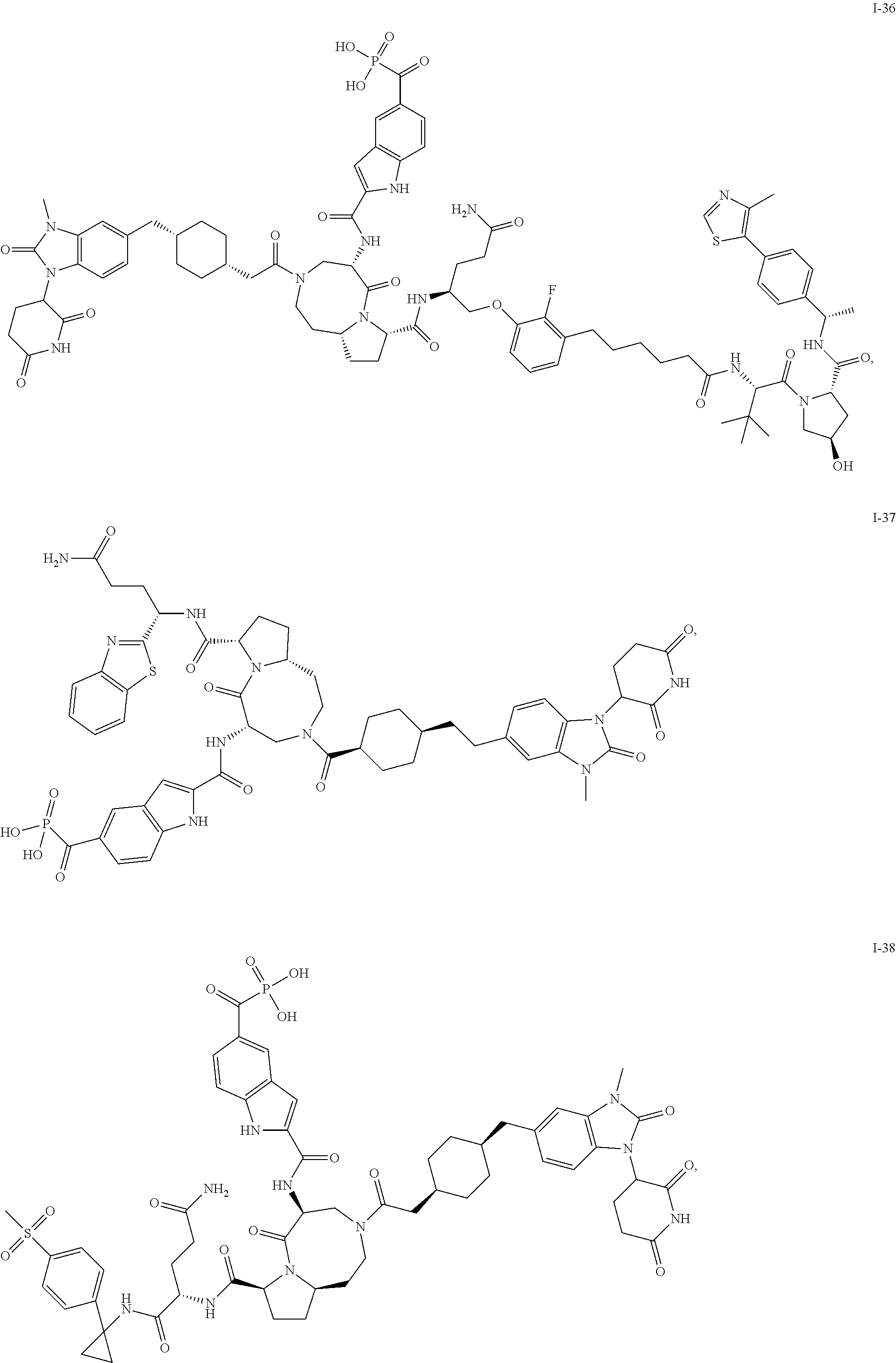 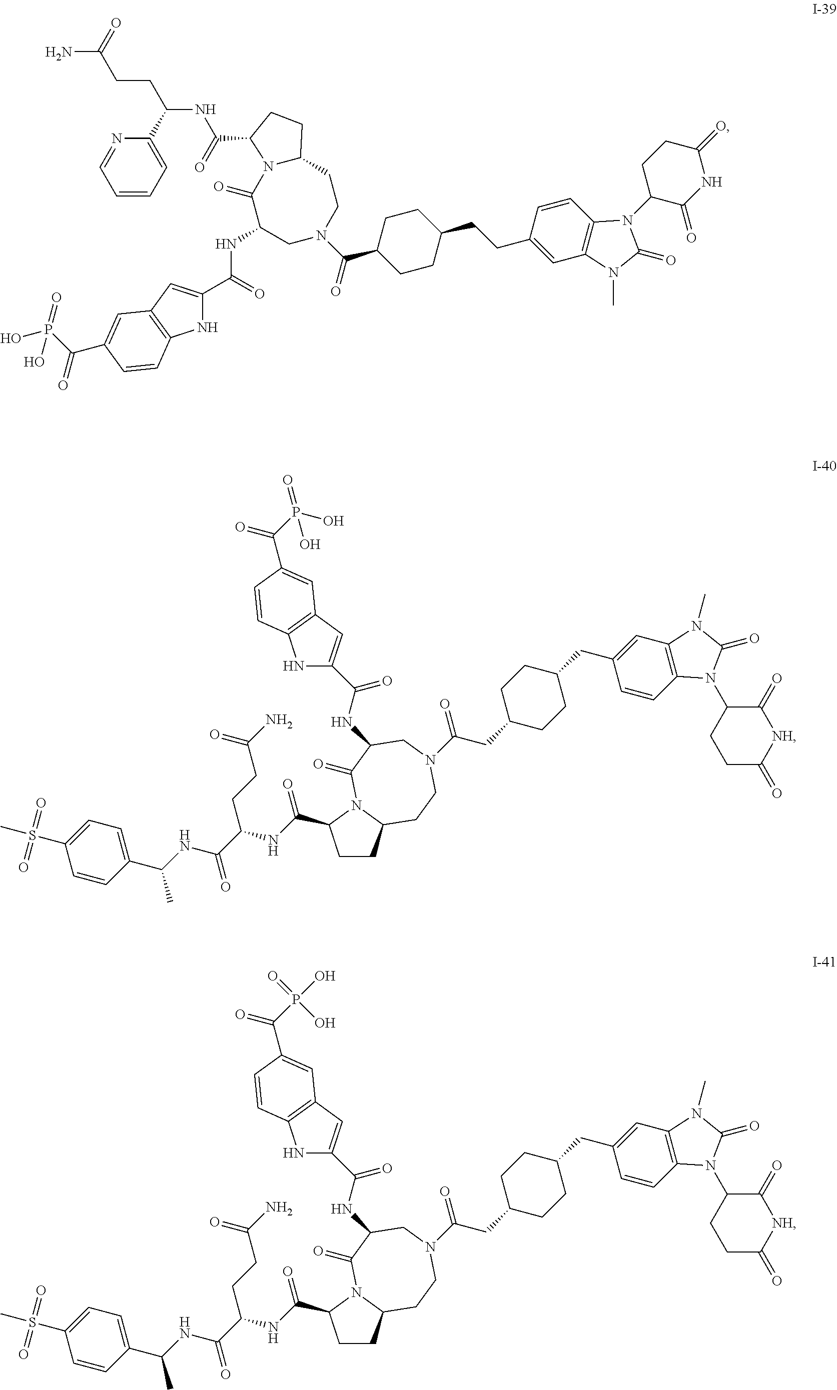 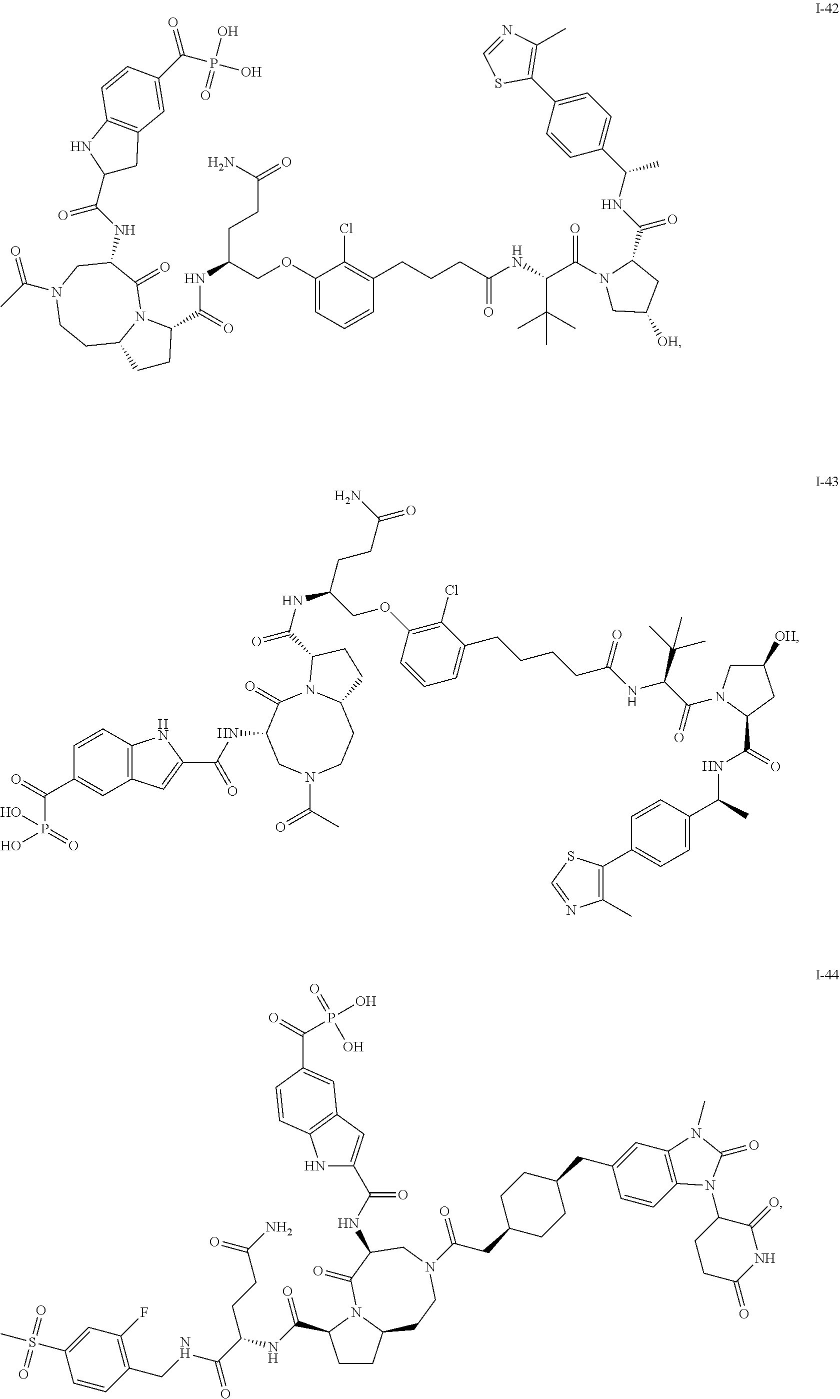 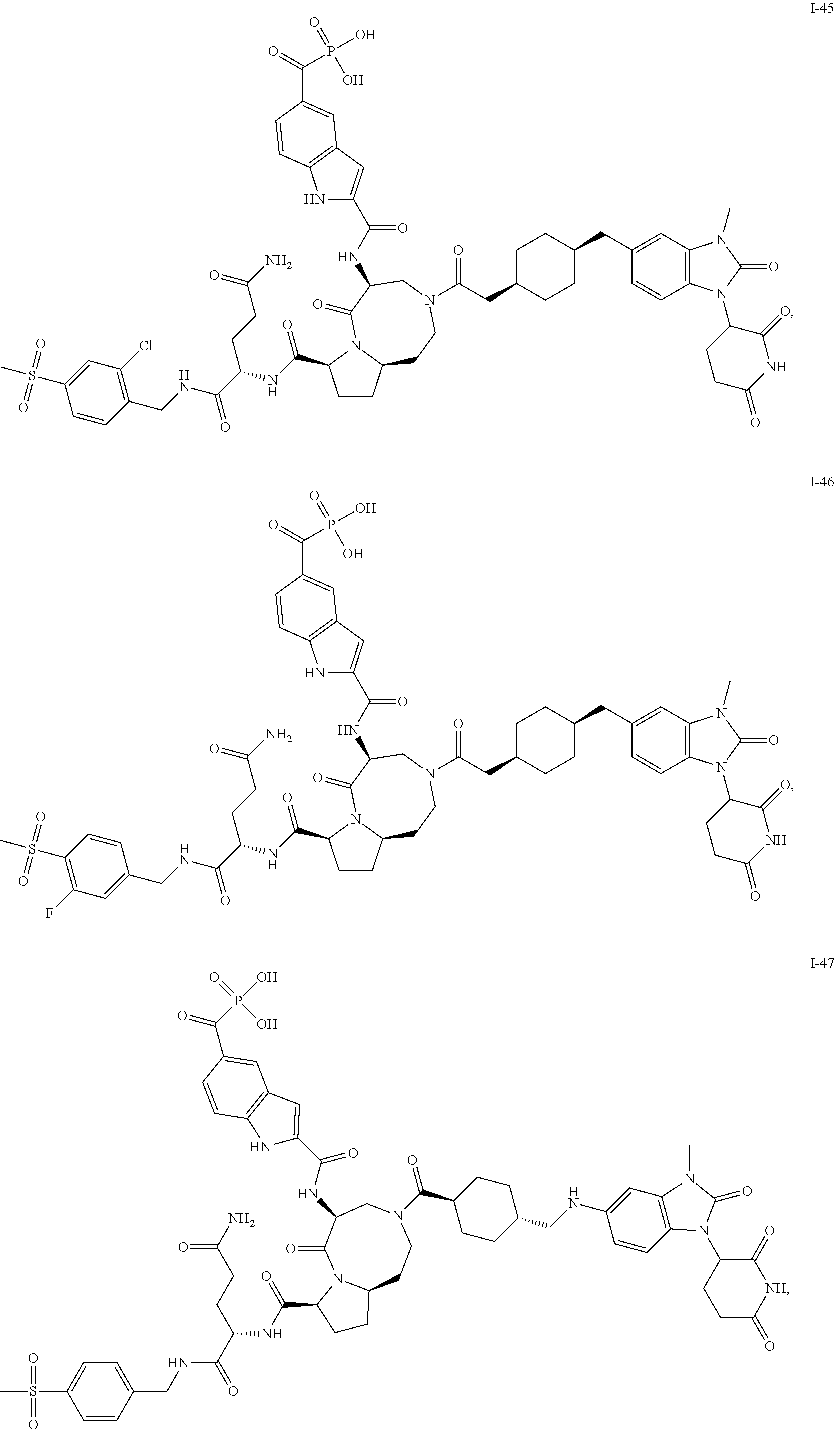 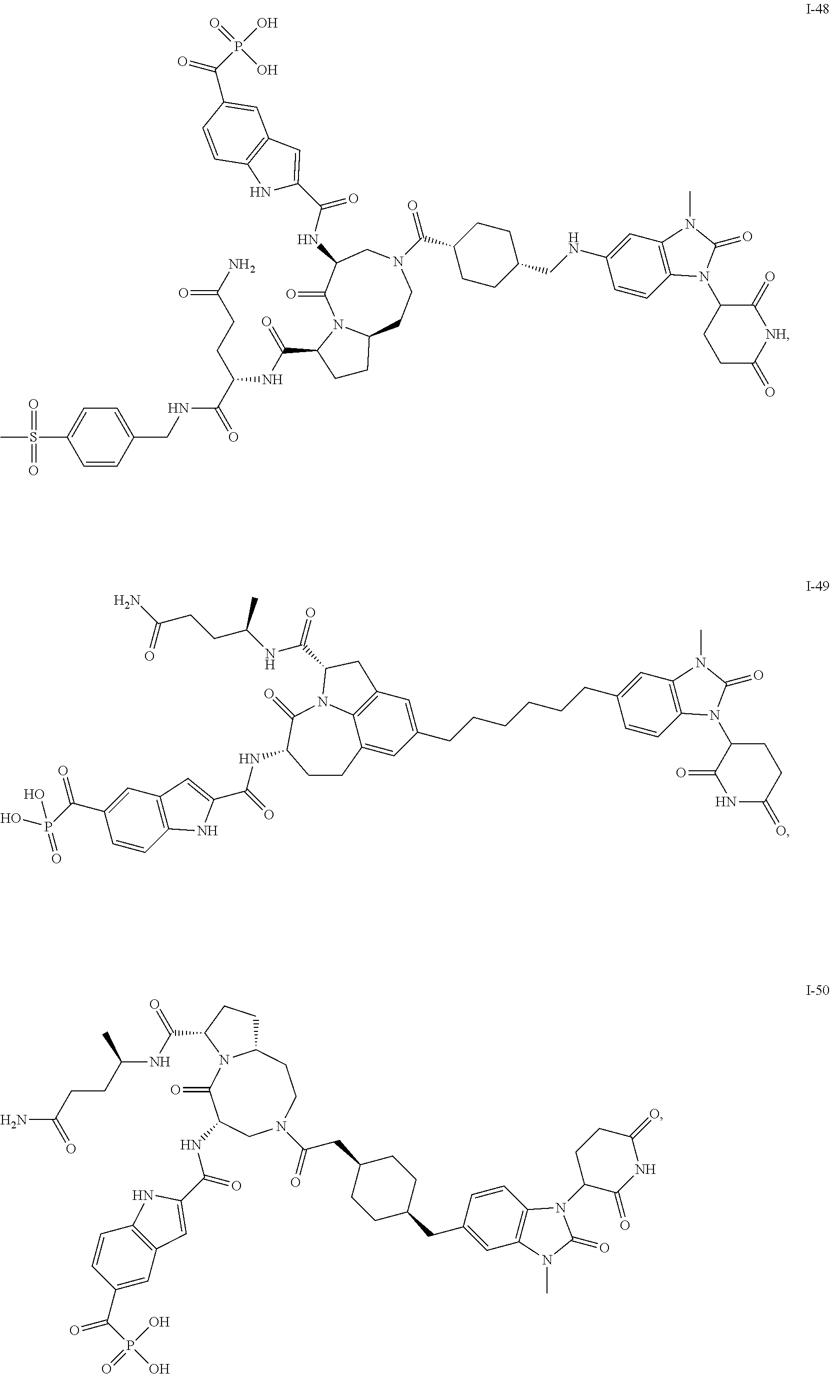 [SMILES: Cn1c(=O)n(C2CCC(=O)NC2=O)c2ccc(NC[C@H]3CC[C@@H](C(=O)N4CC[C@H]5CC[C@@H](C(=O)N[C@@H](CCC(N)=O)C(=O)NCc6ccc(S(C)(=O)=O)cc6)N5C(=O)[C@@H](NC(=O)c5cc6cc(C(=O)P(=O)(O)O)ccc6[nH]5)C4)CC3)cc21 ; C[C@H](CCC(N)=O)NC(=O)[C@@H]1Cc2cc(CCCCCCc3ccc4c(c3)n(C)c(=O)n4C3CCC(=O)NC3=O)cc3c2N1C(=O)[C@@H](NC(=O)c1cc2cc(C(=O)P(=O)(O)O)ccc2[nH]1)CC3 ; C[C@H](CCC(N)=O)NC(=O)[C@@H]1CC[C@@H]2CCN(C(=O)C[C@H]3CC[C@@H](Cc4ccc5c(c4)n(C)c(=O)n5C4CCC(=O)NC4=O)CC3)C[C@H](NC(=O)c3cc4cc(C(=O)P(=O)(O)O)ccc4[nH]3)C(=O)N21] 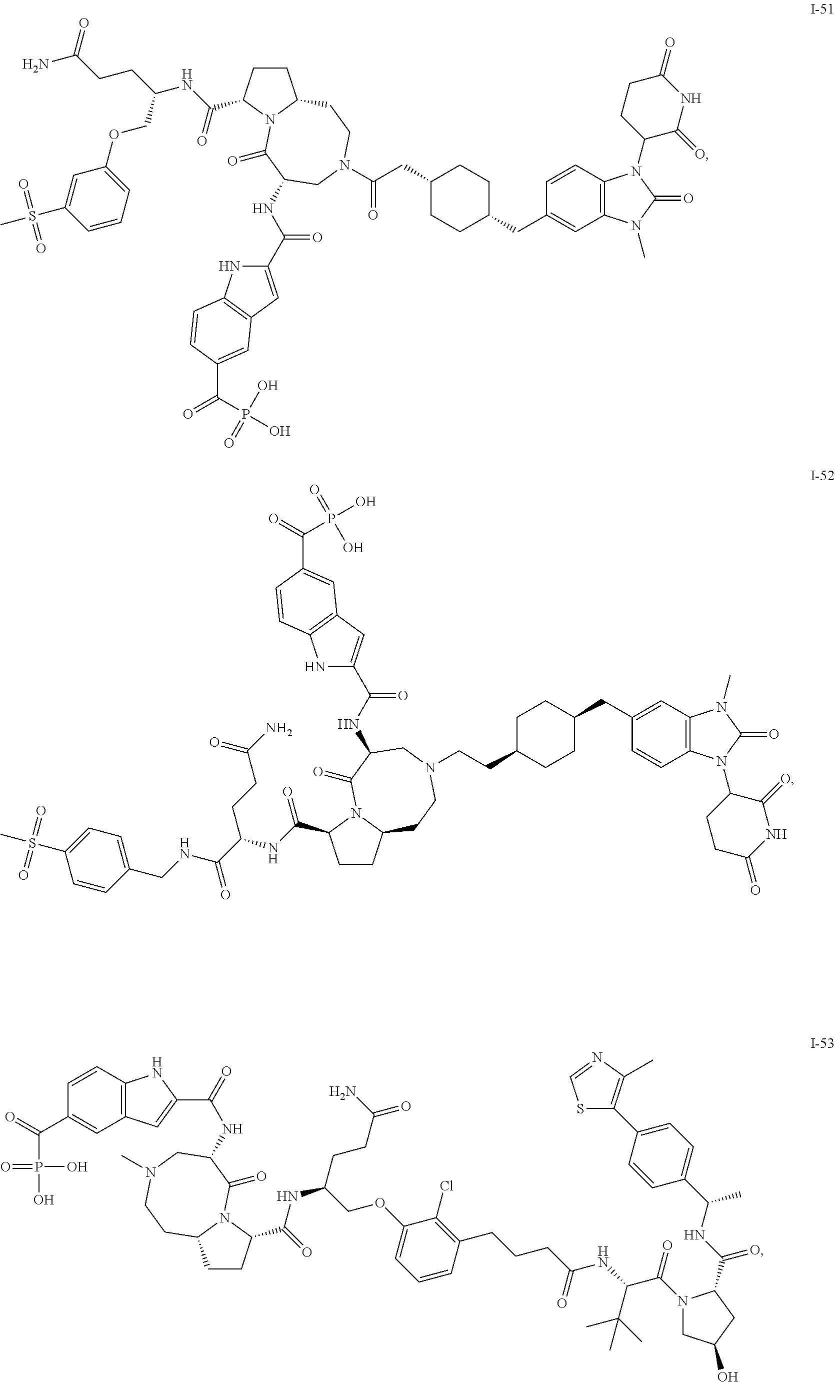 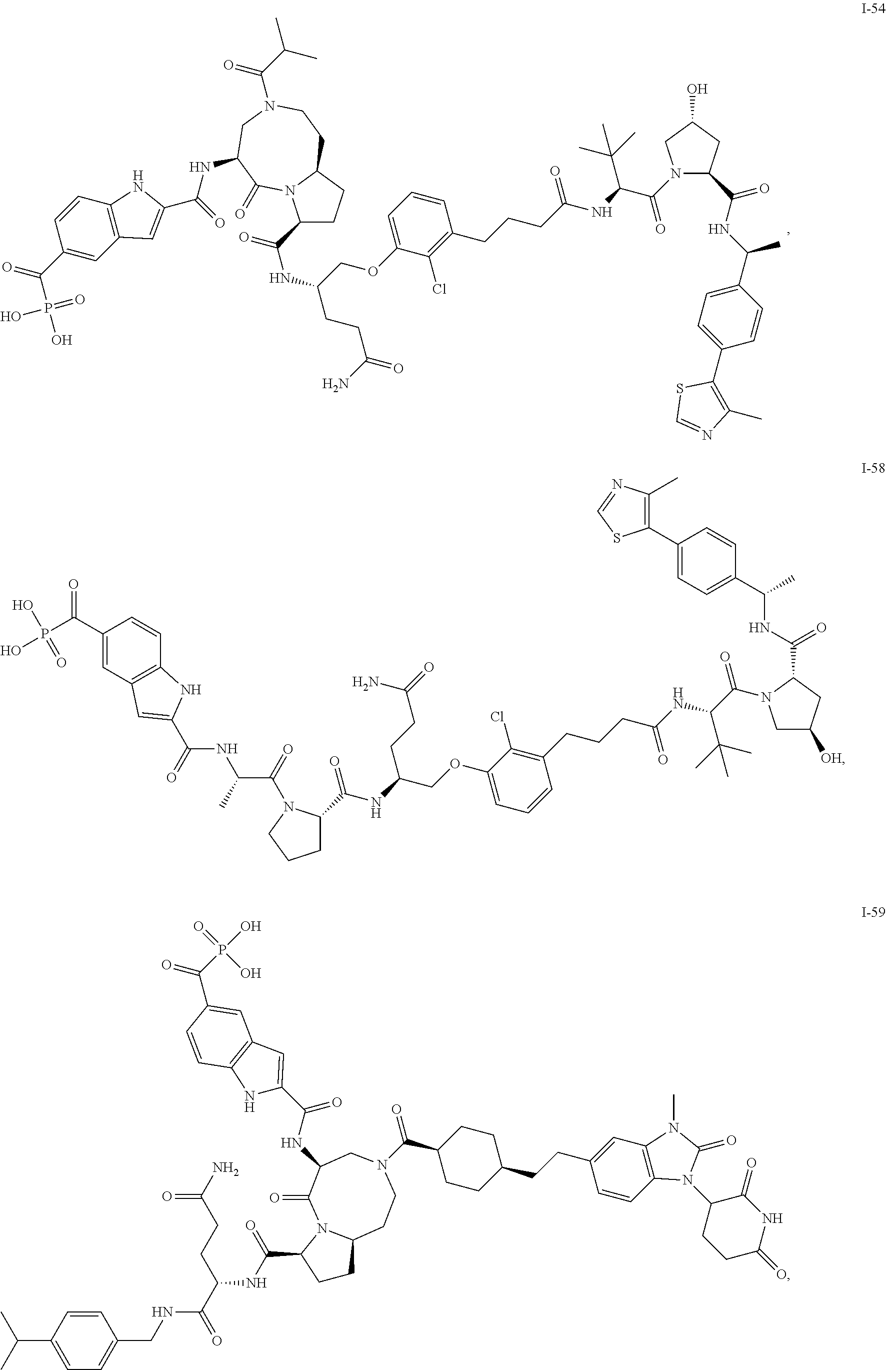 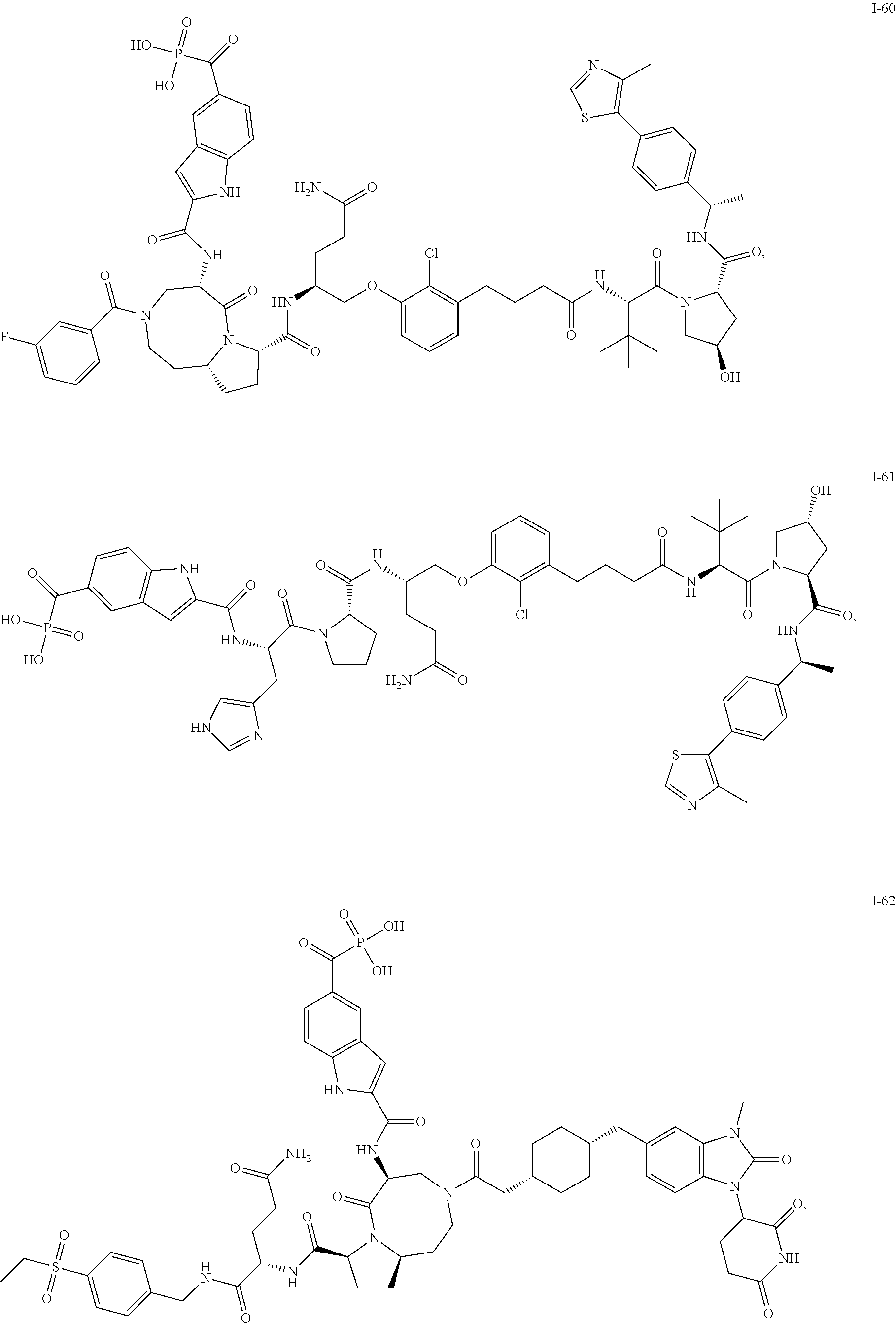 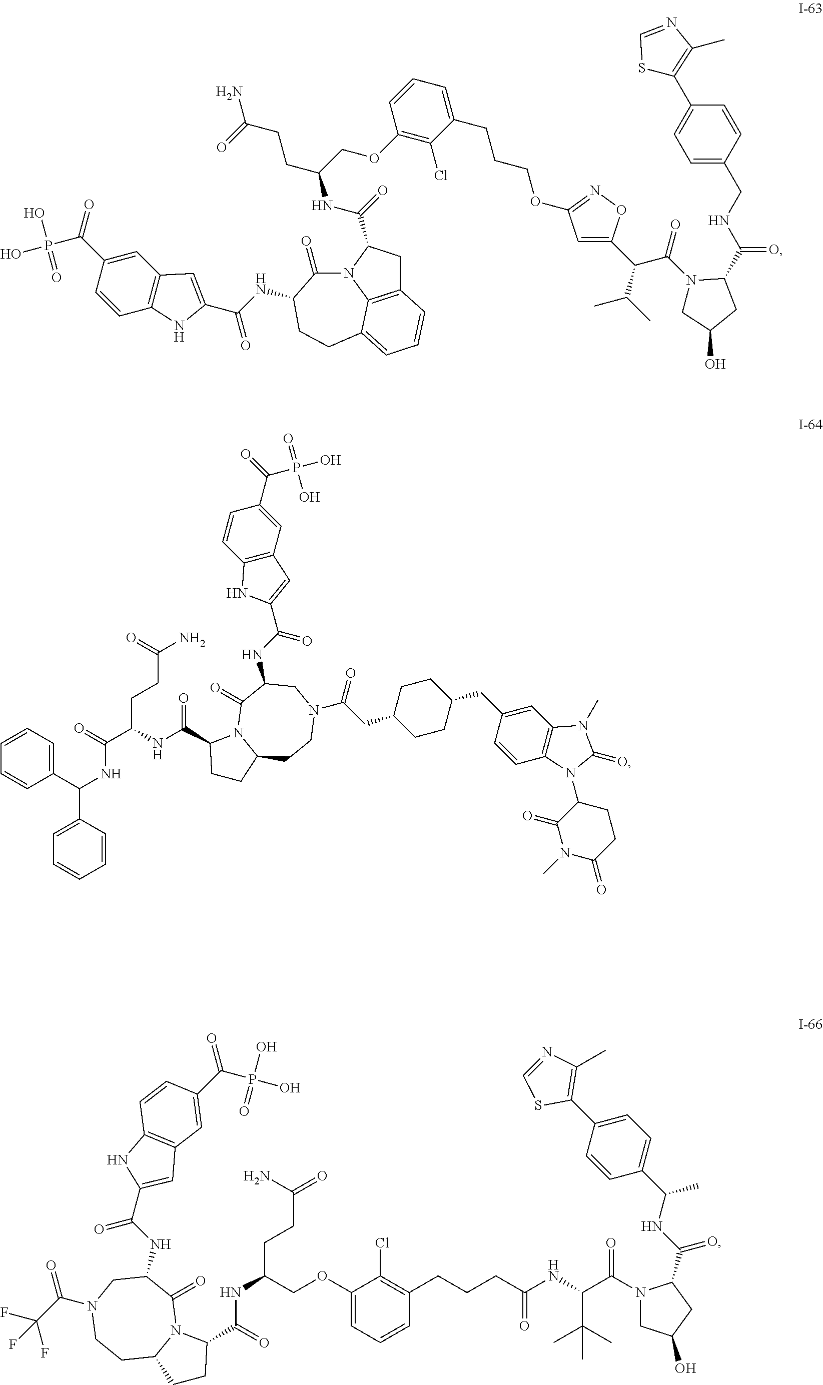 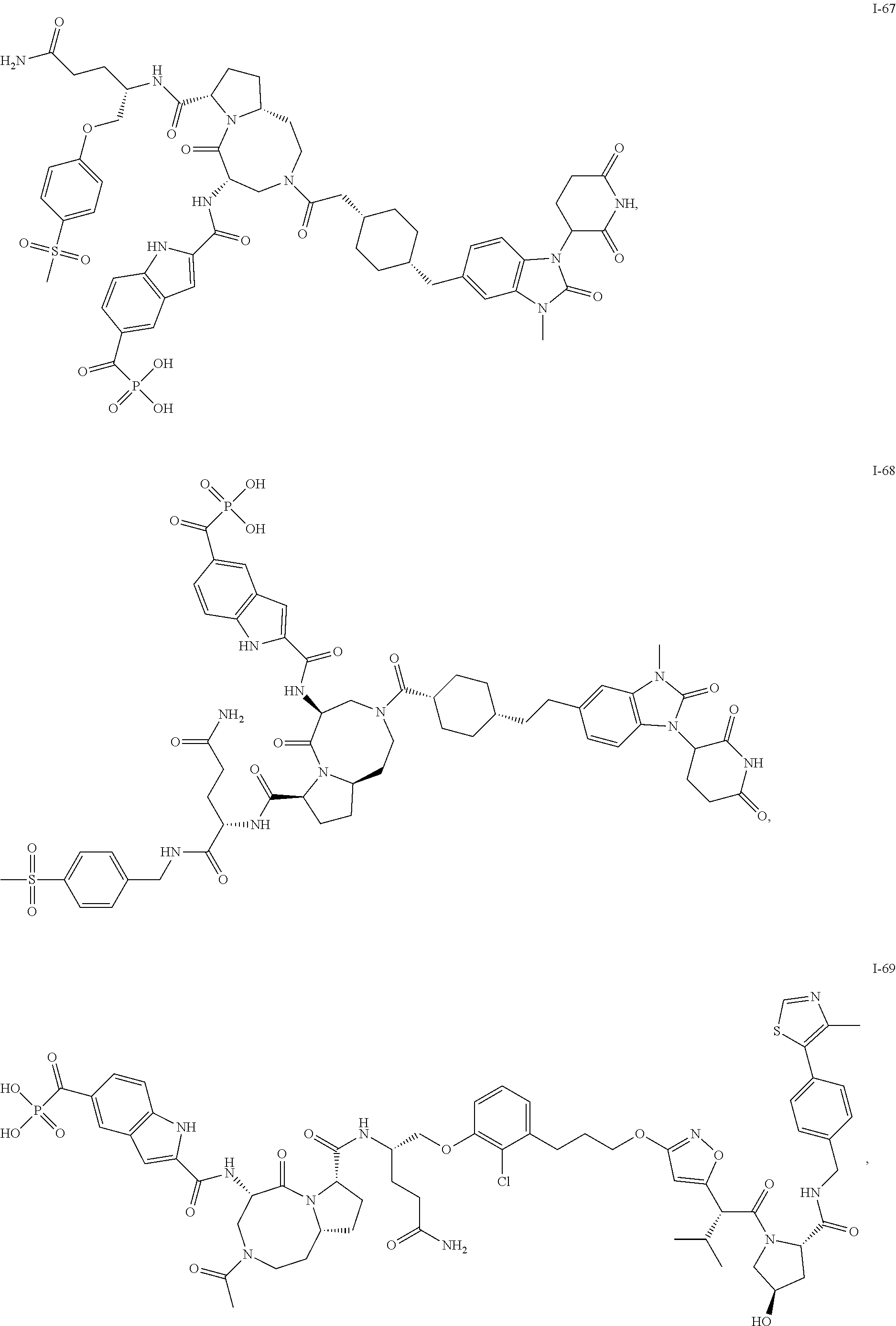 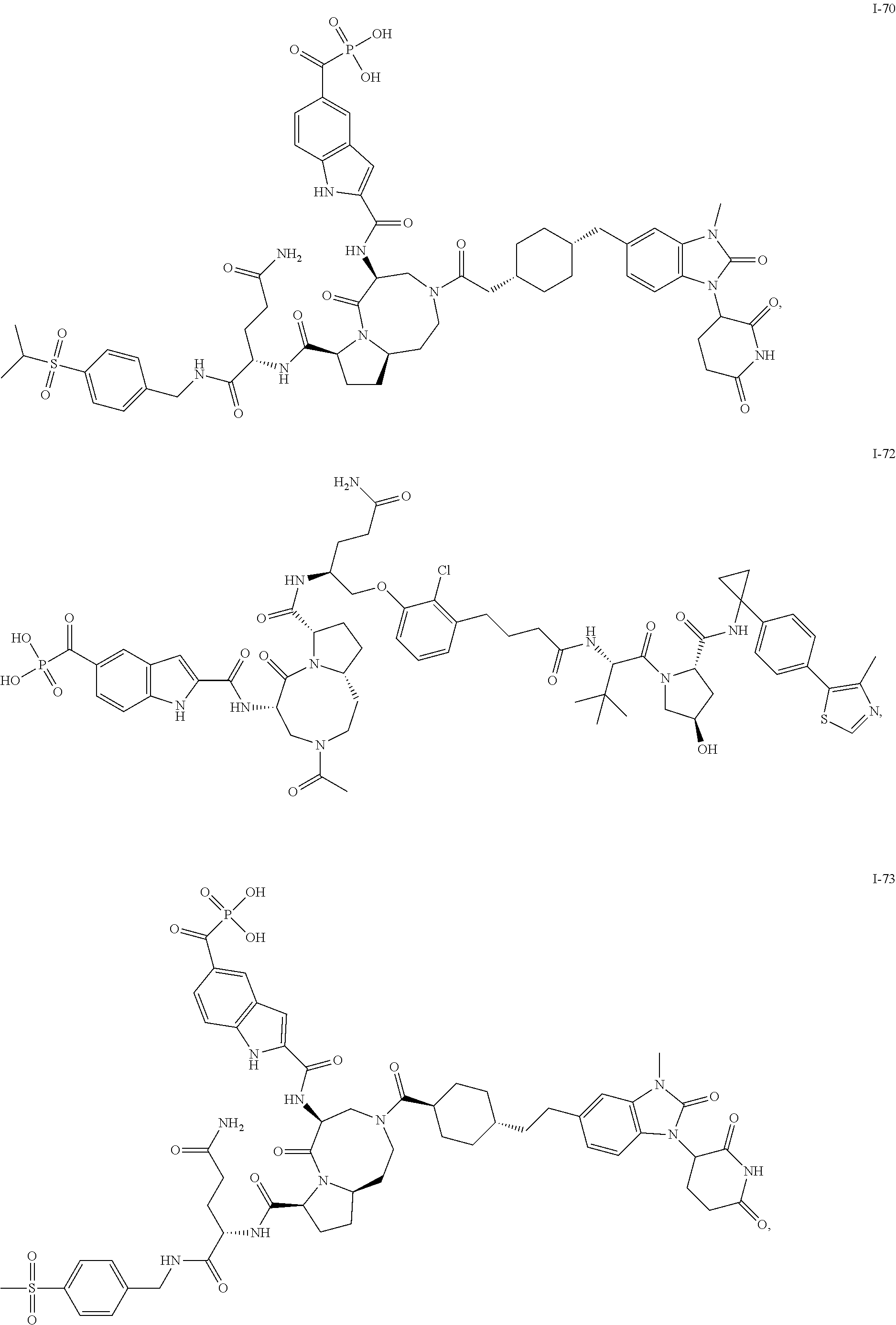 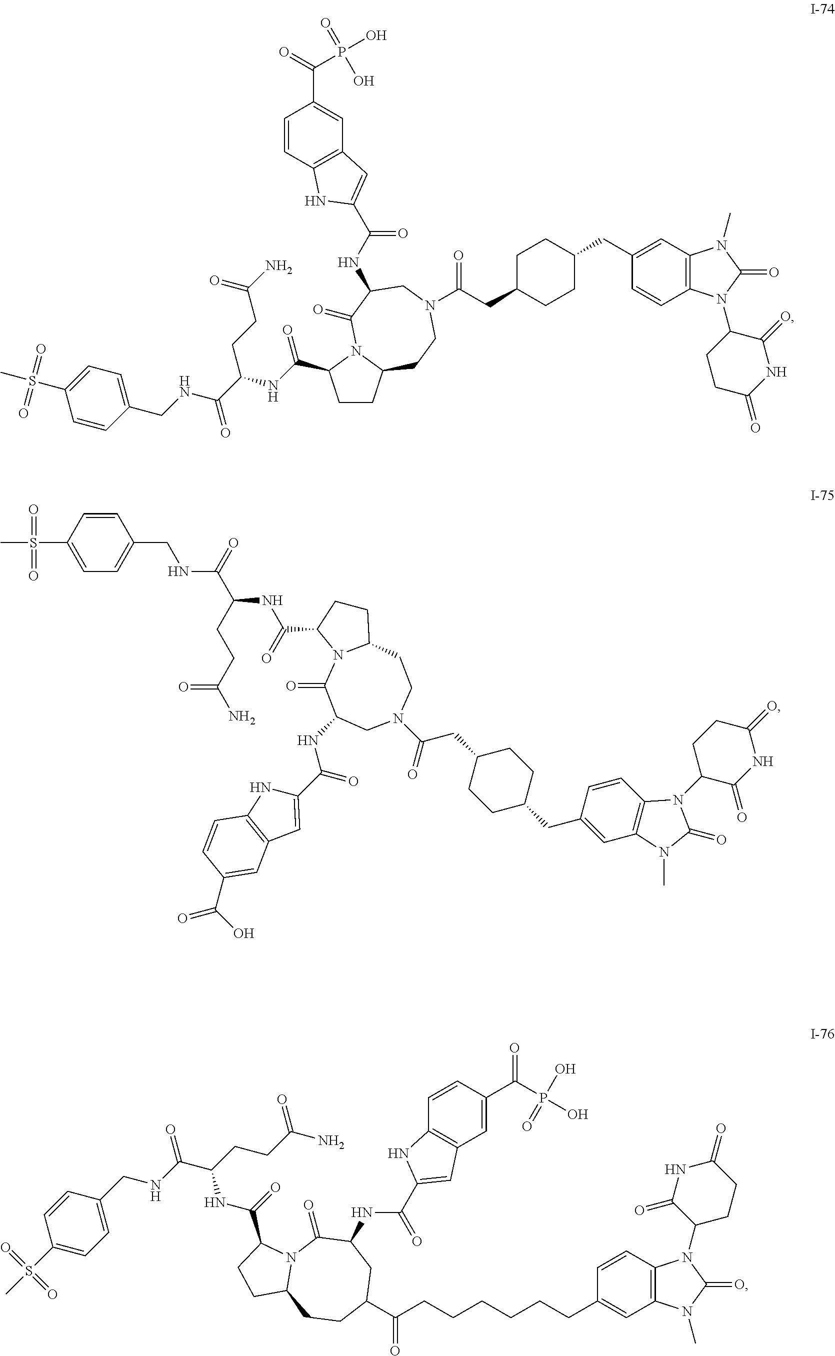 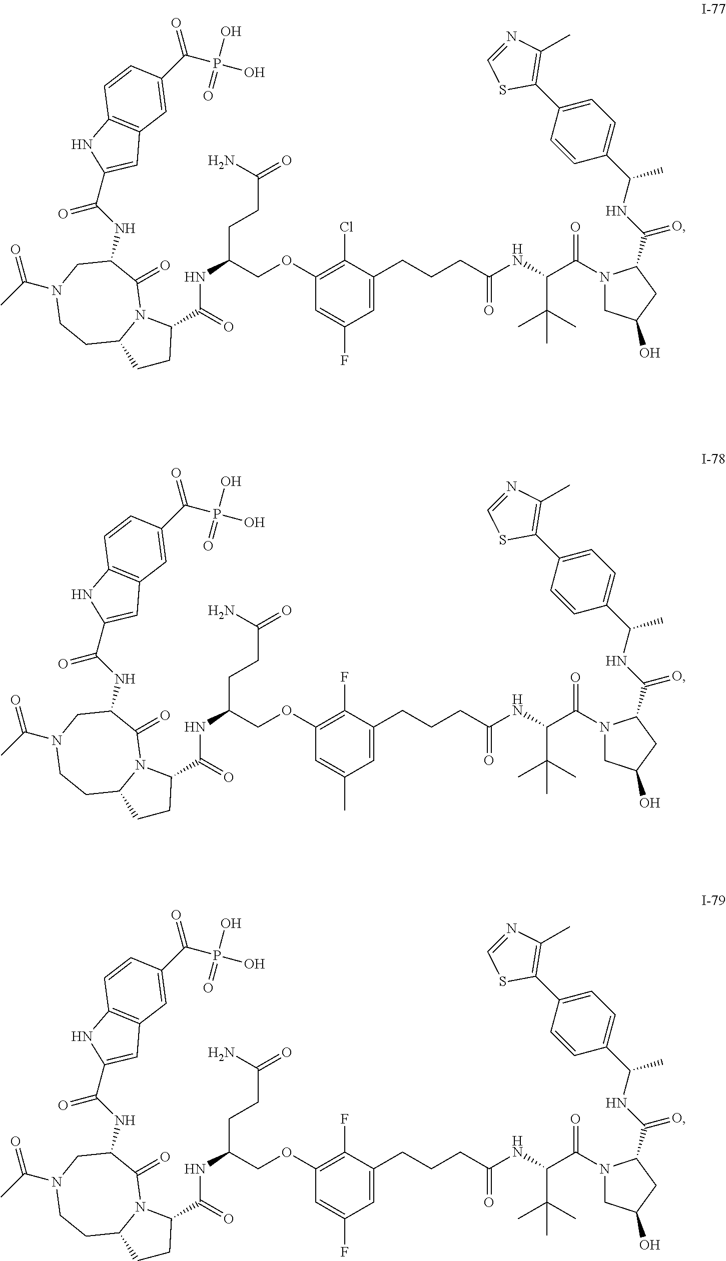 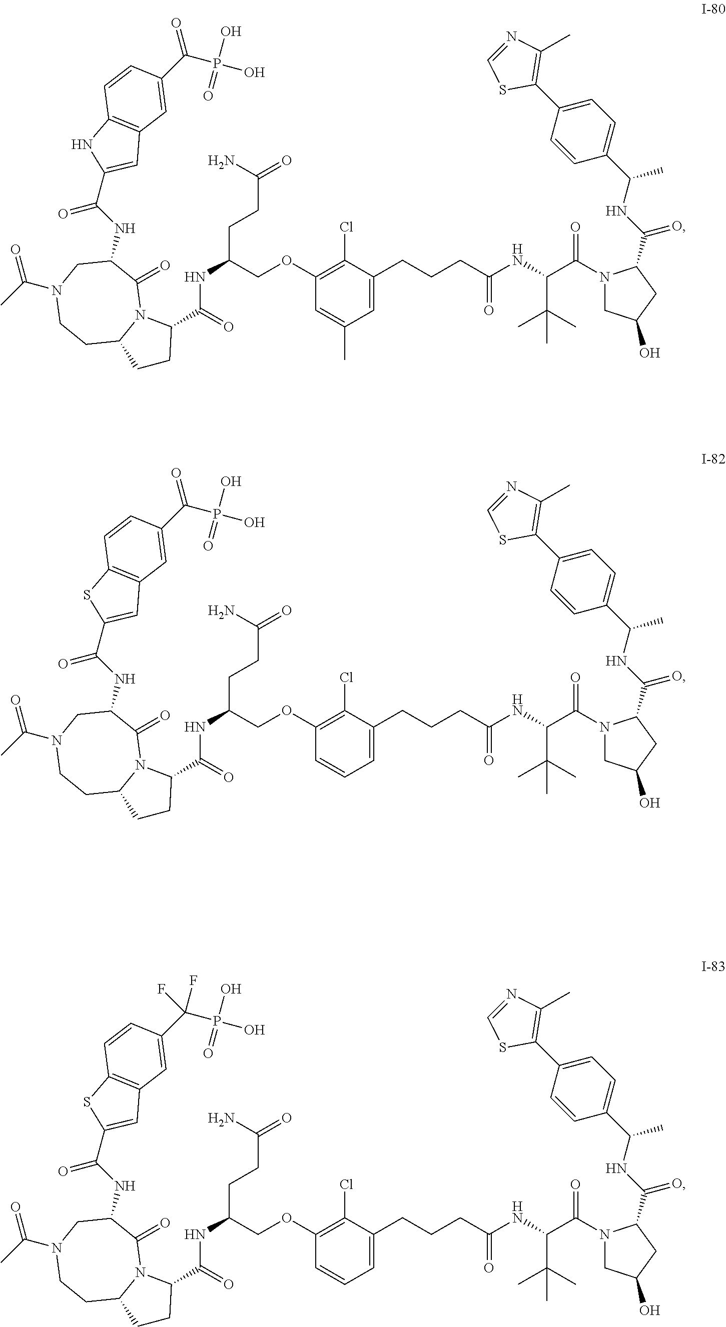 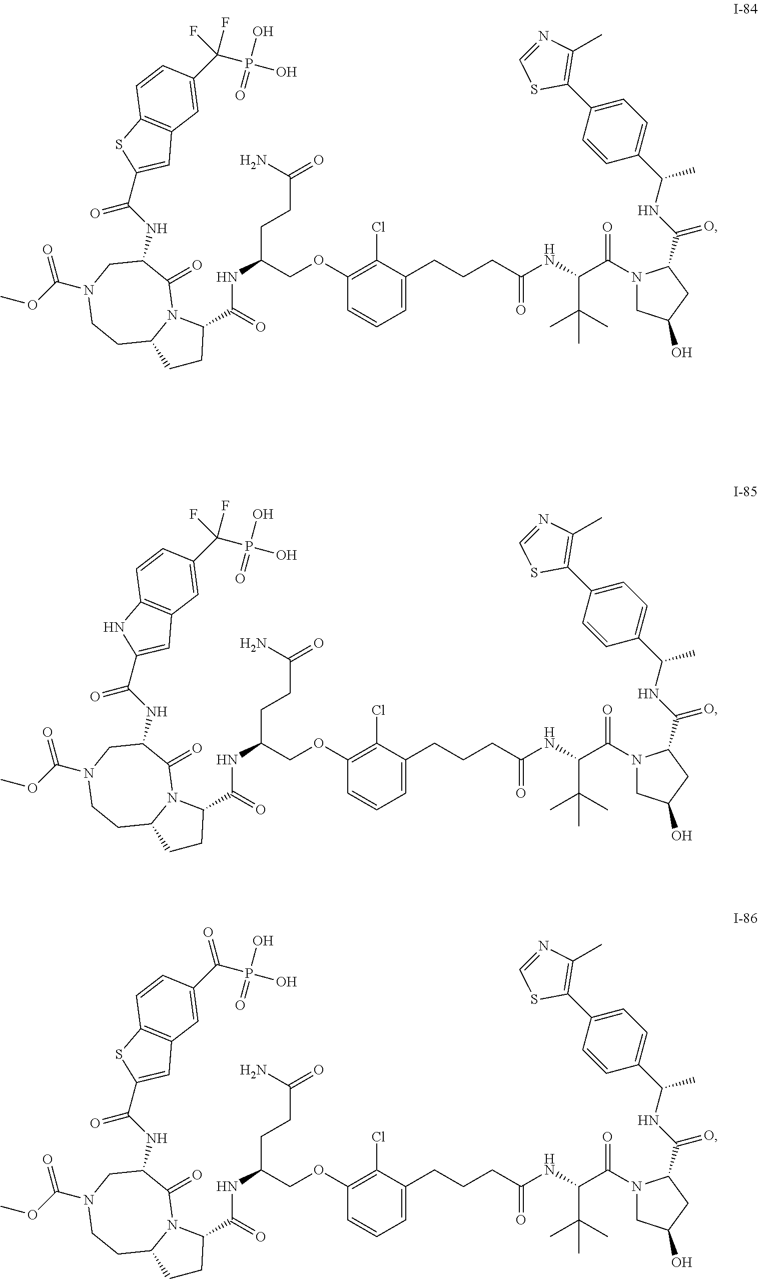 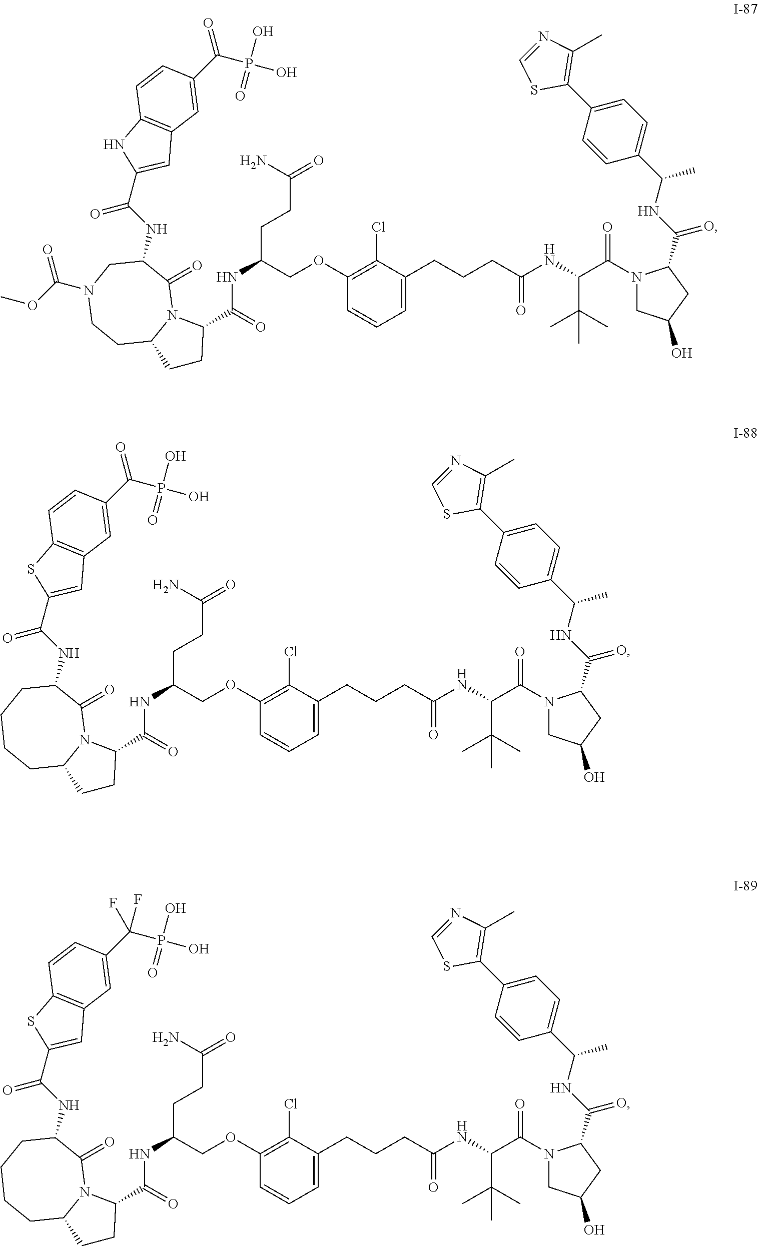 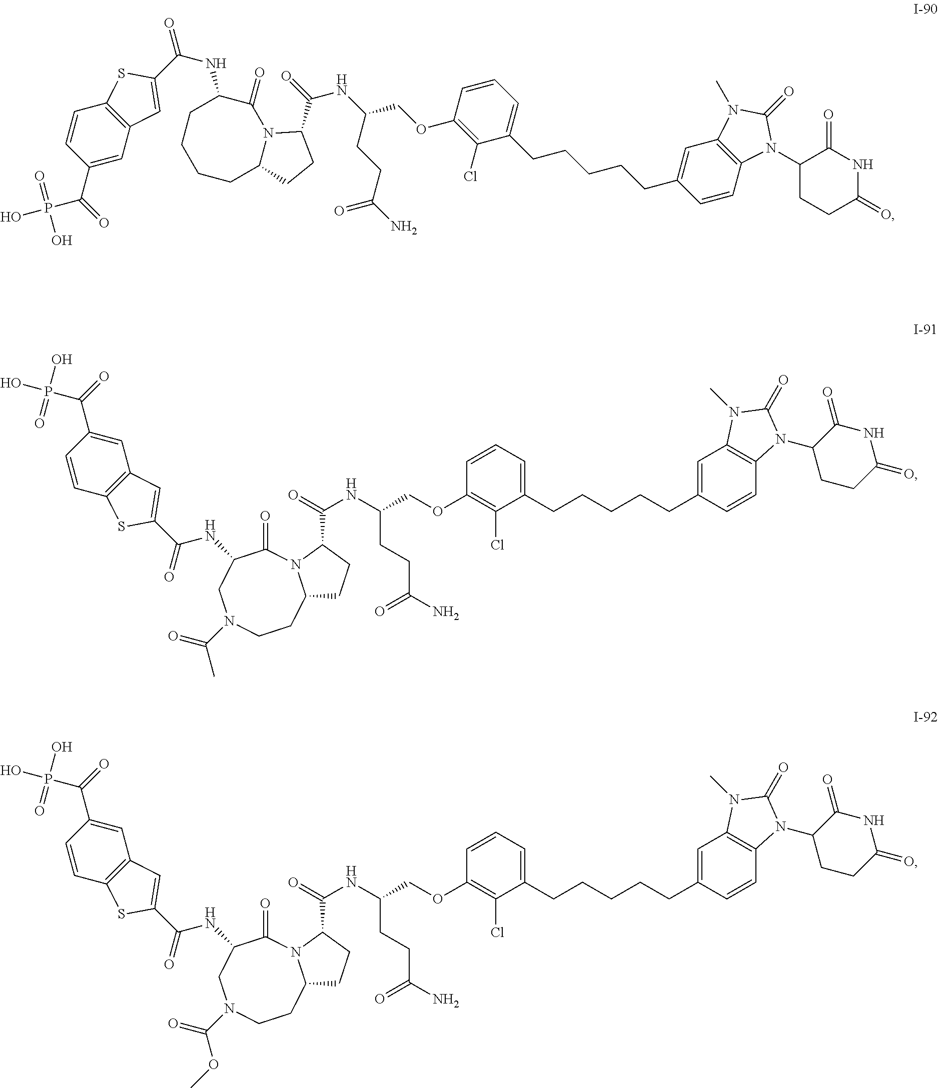 [SMILES: Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(=O)P(=O)(O)O)ccc7s6)C(=O)N54)c3Cl)cc21 ; CC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(=O)P(=O)(O)O)ccc3s2)C1 ; COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(=O)P(=O)(O)O)ccc3s2)C1] 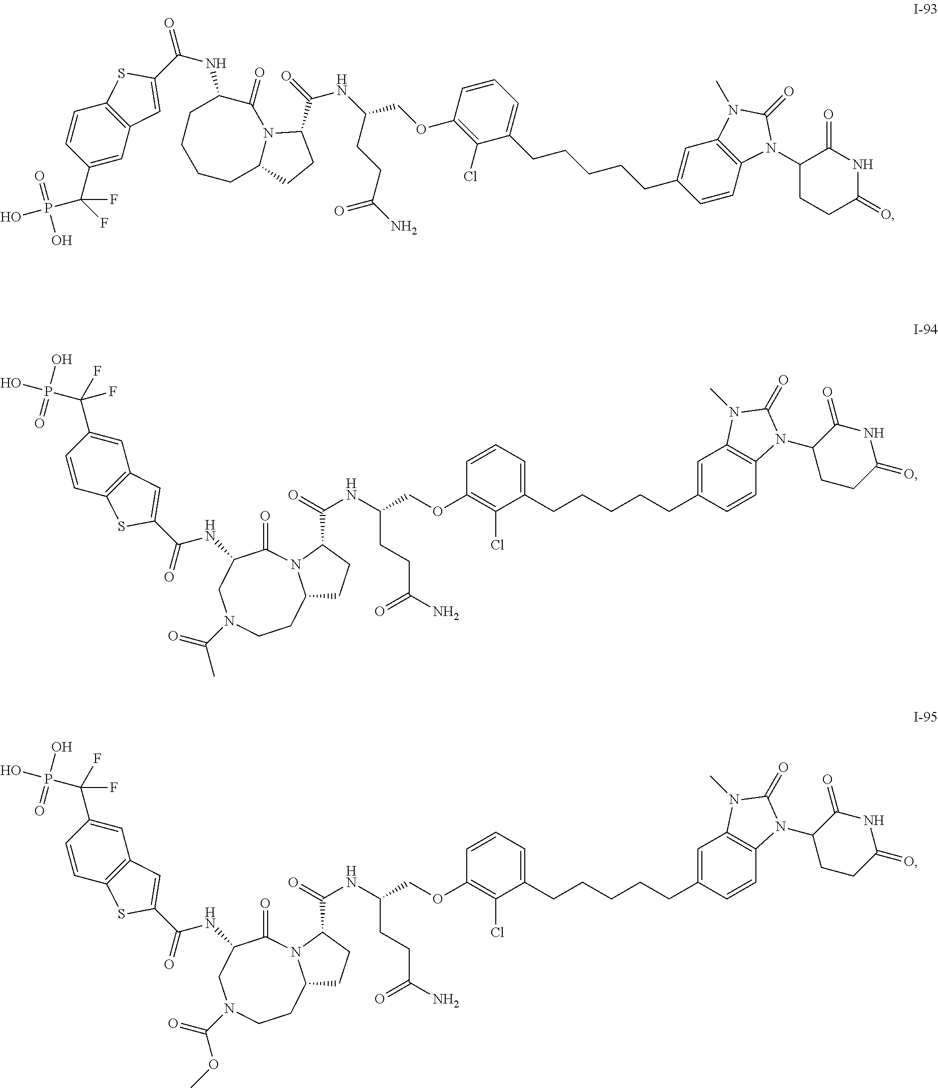 [SMILES: Cn1c(=O)n(C2CCC(=O)NC2=O)c2ccc(CCCCCc3cccc(OC[C@H](CCC(N)=O)NC(=O)[C@@H]4CC[C@@H]5CCCC[C@H](NC(=O)c6cc7cc(C(F)(F)P(=O)(O)O)ccc7s6)C(=O)N54)c3Cl)cc21 ; CC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(F)(F)P(=O)(O)O)ccc3s2)C1 ; COC(=O)N1CC[C@H]2CC[C@@H](C(=O)N[C@@H](CCC(N)=O)COc3cccc(CCCCCc4ccc5c(c4)n(C)c(=O)n5C4CCC(=O)NC4=O)c3Cl)N2C(=O)[C@@H](NC(=O)c2cc3cc(C(F)(F)P(=O)(O)O)ccc3s2)C1] 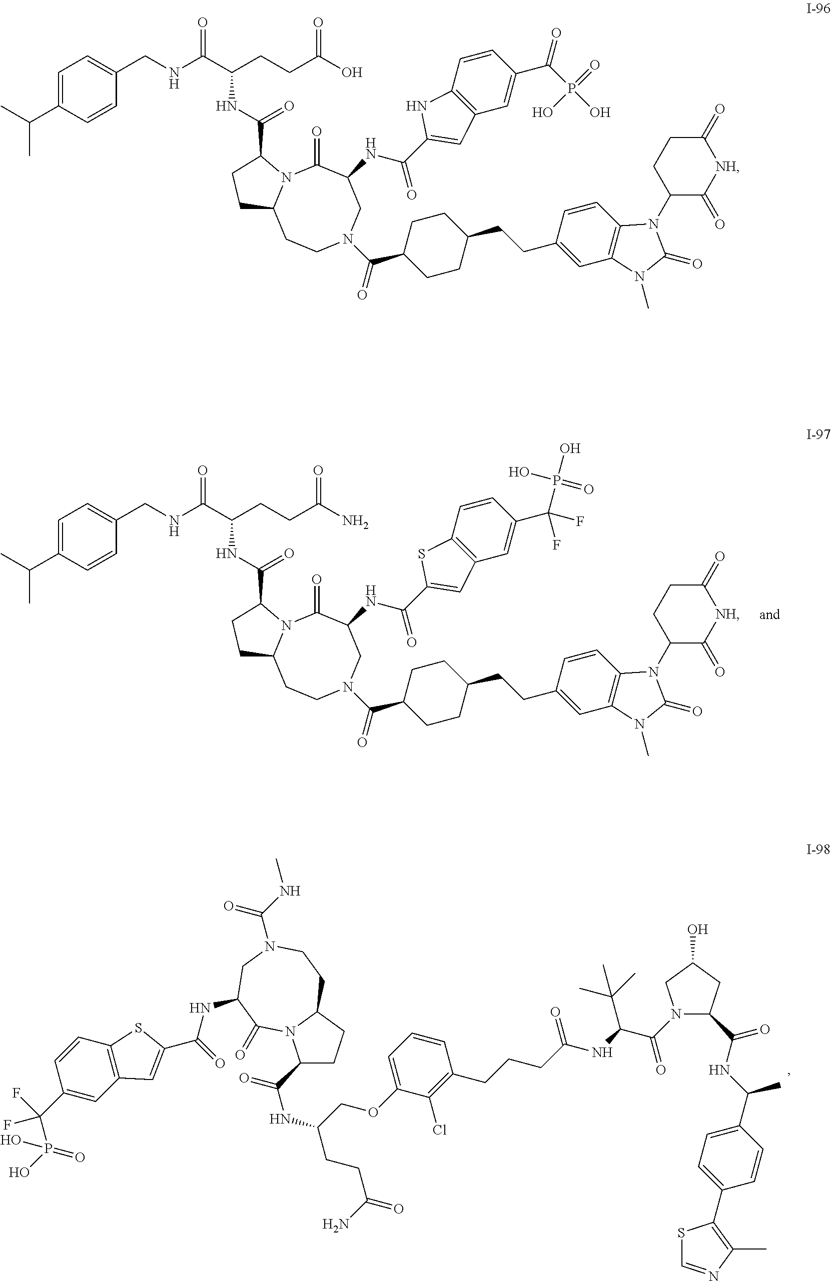
or a pharmaceutically acceptable salt thereof.

11. A pharmaceutical composition comprising a compound of claim 3, and a pharmaceutically acceptable carrier, adjuvant, or vehicle.

12. The pharmaceutical composition of claim 11, further comprising an additional therapeutic agent.

13. A method of degrading STAT3 protein in a patient or biological sample comprising administering to said patient, or contacting said biological sample with a compound of claim 3, or a pharmaceutical composition thereof.

14. A method of treating an STAT3-mediated disorder, disease, or condition in a patient comprising administering to said patient a compound of claim 3, or a pharmaceutical composition thereof.

15. The method of claim 14, further comprising administration of an additional therapeutic agent.

16. The method of claim 14, wherein the STAT3-mediated disorder, disease or condition is selected from a cancer, a neurodegenerative disease, a viral disease, an autoimmune disease, an inflammatory disorder, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, and a CNS disorder.

17. The method of claim 16, wherein the cancer is selected from glioma, breast cancer, prostate cancer, head and neck squamous cell carcinoma, skin melanomas, ovarian cancer, malignant peripheral nerve sheath tumors (MPNST), and pancreatic cancer.

18. The method of claim 17, wherein the breast cancer is triple negative breast cancer.

19-23. (canceled)
